(19) United States
(12) Patent Application Publication
(10) Pub. No.: US 2025/0248979 A1
(43) Pub. Date: Aug. 7, 2025

(54) NOVEL GPX4 INHIBITORS AND USES THEREOF

(71) Applicant: THE TRUSTEES OF COLUMBIA UNIVERSITY IN THE CITY OF NEW YORK, New York, NY (US)

(72) Inventors: BRENT R. STOCKWELL, New York, NY (US); Annie LIN, New York, NY (US); Farhad FOROUHAR, New York, NY (US); Qian WANG, New York, NY (US); Vasiliki POLYCHRONIDOU, New York, NY (US)

(21) Appl. No.: 19/093,166

(22) Filed: Mar. 27, 2025

Related U.S. Application Data
(63) Continuation-in-part of application No. PCT/US23/75356, filed on Sep. 28, 2023.
(60) Provisional application No. 63/496,358, filed on Apr. 14, 2023.
(60) Provisional application No. 63/432,510, filed on Dec. 14, 2022.
(60) Provisional application No. 63/410,788, filed on Sep. 28, 2022.

(51) Int. Cl. A61K 31/437; C07D 231/54; C07D 401/04; C07D 403/04; C07D 405/04; C07D 409/04; C07D 413/10; C07D 413/14; C07D 487/04

(52) U.S. Cl. CPC A61K 31/437; C07D 231/54; C07D 401/04; C07D 403/04; C07D 405/04; C07D 409/04; C07D 413/10; C07D 413/14; C07D 487/04

(57) ABSTRACT

The present disclosure provides, inter alia, compounds to modulate GPX4 activity. Also provided are pharmaceutical compositions containing such compounds. Further provided are methods for treating or ameliorating the effects of a cancer in a subject, methods of modulating GPX activity in a subject, methods of inducing ferroptosis in a cell, and methods for treating or ameliorating the effects of a cancer in a subject using the compounds or composition in combination with other therapeutic agents.

### CROSS REFERENCE TO RELATED APPLICATIONS

The present application is a continuation-in-part of PCT international application no. PCT/US2023/075356, filed on Sep. 28, 2023, which claims benefit of U.S. Provisional Patent Application Ser. No. 63/496,358, filed on Apr. 14, 2023, U.S. Provisional Patent Application Ser. No. 63/432,510, filed on Dec. 14, 2022, and U.S. Provisional Patent Application Ser. No. 63/410,788, filed on Sep. 28, 2022. The contents of above applications are incorporated by reference herein in their entireties.

### GOVERNMENT FUNDING

This disclosure was made with government support under grant no. CA209896, awarded by National Institutes of Health. The government has certain rights in the disclosure.

### FIELD OF DISCLOSURE

The present disclosure provides, inter alia, compounds to modulate GPX4 activity. Also provided are pharmaceutical compositions containing the compounds, as well as methods of using such compounds and compositions.

### BACKGROUND OF THE DISCLOSURE

Cancer cells are dependent on their lipid composition for establishing and modulating membrane structural integrity, morphology, metabolism, migration, invasiveness, and other functions. For example, among the thousands of lipid species that compose eukaryotic cell membranes, the abundance and localization of polyunsaturated-fatty-acid-(PUFA)-containing phospholipids (PUFA-PLs) is a major factor in determining the fluidity of cell membranes (Agmon et al. 2017). Since the cis conformation of double bonds in PUFA-PLs hinders efficient stacking of fatty acyl tails, elevated levels of PUFA-PLs contribute to increasing membrane fluidity and thinning (Agmon et al. 2018). PUFA-PLs are, however, susceptible to peroxidation via iron-catalyzed reaction with molecular oxygen at bis-allylic positions, catalyzed by lipoxygenases and labile iron (Yang et al. 2016). Thus, some cancer cells depend on a critical network of proteins to eliminate their PUFA-PL peroxides; a key protein at the center of this defense network is the selenoprotein glutathione peroxidase 4 (GPX4). When GPX4 activity is compromised, lipid peroxidation can cause ferroptosis (Stockwell et al. 2017), an oxidative, iron-dependent form of non-apoptotic cell death (Dixon et al. 2012). Ferroptosis acts as a natural tumor suppressive and immune surveillance mechanism, and can be induced by exogenous agents in cells that are addicted to GPX4 (Dixon and Stockwell, 2019). Cancer cells from tissues of diverse origins have been screened for their sensitivity to ferroptosis-inducing compounds (Viswanathan et al. 2017). It has been found that ferroptosis inducers, including GPX4 inhibitors, selectively target cancers with a mesenchymal or otherwise drug-resistant signature (Viswanathan et al. 2017). Consistent with the mesenchymal state being associated with drug resistance, an independent study on persister cancer cells, which are proposed to escape from conventional cytotoxic treatment through a dormant state and then revive to cause tumor relapse, revealed a similar selective dependency on GPX4 (Hangauer et al. 2017).

Examination of persister cells also revealed upregulation of mesenchymal markers and downregulation of epithelial markers (Hangauer et al. 2017). Overexpression of mesenchymal state genes is associated with epithelial-mesenchymal transition (EMT). Since EMT increases motility of tumor cells and enables the invasion of primary tumors to distant sites, EMT is a key step in metastasis. EMT also renders cancer cells resistant to apoptosis and chemotherapy (Viswanathan et al. 2017). EMT requires plasma membrane remodeling to increase fluidity, which is associated with elevated biosynthesis of PUFA-PLs. Given that PUFA-PLs are more susceptible to peroxidation than saturated or mono-unsaturated fatty acid PLs, cells in an EMT state have increased dependency on GPX4 to remove these lipid peroxides (Viswanathan et al. 2017). Therefore, cancer cells undergoing EMT that acquire resistance to apoptosis become vulnerable to lipid peroxidation and ferroptosis induced by GPX4 inhibition (Viswanathan et al. 2017). As cancer cells evolve into a high-mesenchymal drug-resistant state and become resistant to apoptosis, one may selectively target such cells through ferroptosis; the most effective compounds in this context are GPX4 inhibitors (Viswanathan et al. 2017). For example, in-vivo xenografts of GPX4-knockout high-mesenchymal therapy-resistant melanoma regressed after cessation of ferrostatin-1 (a lipophilic antioxidant discovered in the Stockwell Lab that suppresses the loss of GPX4) and did not relapse after ceasing dabrafenib and trametinib treatment, while wt GPX4 xenografts continued to grow in both experiments (Viswanathan et al. 2017). GPX4 inhibitors are selectively lethal to persister and EMT cancer cells, with minimal effects on parental cells and non-transformed cells, suggesting that addiction to GPX4 creates a large therapeutic window.

Accordingly, there is a need for developing GPX4 inhibitors for the treatment of aggressive drug-resistant cancers and other GPX4-associated diseases. This disclosure is directed to meeting these and other needs.

### SUMMARY OF THE DISCLOSURE

One of the most pressing problems in oncology is metastatic, drug-resistant cancers. Indeed, most deaths of cancer patients are caused by aggressive, metastatic, drug-resistant cancers. A surprising finding is that as cancers evolve into aggressive and drug-resistant forms, they acquire an exquisite sensitivity to GPX4 inhibition. These data provide the tantalizing possibility that the most aggressive neoplastic diseases can be treated through the use of GPX4 inhibitors, and that the ideal patients for treatment with these inhibitors are end-stage patients that have exhausted other therapeutic options. In 2012, we reported the existence of a new form of tumor suppressive cell death, ferroptosis (Dixon et al. 2012). In 2014, we discovered that the key negative regulator of ferroptosis was the lipid repair enzyme GPX4, demonstrating that GPX4 functions in ferroptosis in a manner analogous to how the oncogene Bcl-2 functions in apoptosis (Yang et al. 2014). We discovered the first GPX4 inhibitor—the nanomolar potency small molecule RSL3 (Yang et al. 2014). In 2017, we reported that cancer cells that have undergone epithelial-to-mesenchymal (EMT) transition become hypersensitive to ferroptosis, and to GPX4 inhibitors (Viswanathan et al. 2017). We also discovered how RSL3 inhibits GPX4, obtaining a co-crystal structure of RSL3 bound to GPX4, which revealed a novel drug-binding site on GPX4. We propose, inter alia, to exploit this finding to discover drug-like GPX4 inhibitors with favorable ADMET properties that can be developed as first in class GPX4 inhibitors for drug-resistant cancers having a high EMT gene expression signature.

Accordingly, one embodiment of the present disclosure is a compound according to formula (1):

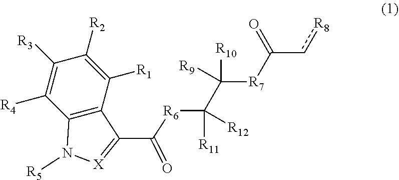

wherein:
a dashed line indicates the presence of an optional double bond;
X is selected from the group consisting of O, N, S, and C;
R₁, R₂, R₃, R₄ and R₅ are independently selected from the group consisting of H, D, —OH, halo, ether, ester, furanyl, indole, indazole, pyrrole, pyrazole, pyridine, pyrimidine, naphthalene, indene, dibenzofuran, benzodioxole, amide, —(O)C(R), —C(O)OR, —NO₂, alkyl, aryl, alkyl-aryl, alkyl-heteroaryl, alkenyl, alkenyl-aryl, alkenyl-heteroaryl, peptide, and polypeptide, wherein the ether, ester, furanyl, indole, indazole, pyrrole, pyrazole, pyridine, pyrimidine, naphthalene, indene, dibenzofuran, benzodioxole, amide, alkyl, aryl, alkyl-aryl, alkyl-heteroaryl, alkenyl, alkenyl-aryl, alkenyl-heteroaryl, peptide, and polypeptide may be optionally substituted with an atom or a group selected from the group consisting of O, N, S, epoxy, —OH, halo, —CN, —NO₂, C₁₋₄alkyl, CF₃, —CF₃O, carbonyl, alkyl-aryl, thiol, methylmethanesulfonamide, methylthiol, dioxane, tetrahydropyzan, morpholinyl, piperazine, sulfonyl, oxazole, oxadiazole, diazole, triazole, amide, ether, ester and combinations thereof; or R₁ and R₂ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₂ and R₃ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₃ and R₄ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof,
R₆ and R₇ are independently selected from the group consisting of C, N, O and S; or R₆ and R₇ may together form a saturated or unsaturated ring structure that may be optionally substituted with an atom or a group from the group consisting of O, N, halo, C₁₋₄alkyl, CF₃, oxazole, oxadiazole, dizaole, triazole, amide, ether, ester and combinations thereof,
R₈ is selected from the group consisting of H, D, C, O, N, alkenyl, alkynyl, ester, oxazole, piperazine, morpholinyl and halo,
R₉, R₁₀, R₁₁ and R₁₂ are independently selected from the group consisting of no atom, H, D, —OH, halo, —CN, —NO₂, C₁₋₄alkyl, CF₃, —CF₃O, carbonyl, aryl, alkyl-aryl, thiol, ether, and ester,
wherein R is selected from the group consisting of H, D, O, N, halo, oxazole, oxadiazole, dizaole, triazole, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of O, N, S, halo, C₁₋₄alkyl, CF₃, —CN and combinations thereof,
or an N-oxide, crystalline form, hydrate, or pharmaceutically acceptable salt thereof.

Another embodiment of the present disclosure is a compound according to formula (2):

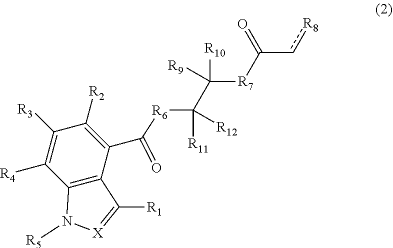

wherein:
a dashed line indicates the presence of an optional double bond;
X is selected from the group consisting of O, N, S, and C;
R₁, R₂, R₃, R₄ and R₅ are independently selected from the group consisting of H, D, —OH, halo, ether, ester, furanyl, indole, indazole, pyrrole, pyrazole, pyridine, pyrimidine, naphthalene, indene, dibenzofuran, benzodioxole, amide, —(O)C(R), —C(O)OR, —NO₂, alkyl, aryl, alkyl-aryl, alkyl-heteroaryl, alkenyl, alkenyl-aryl, alkenyl-heteroaryl, peptide, and polypeptide, wherein the ether, ester, furanyl, indole, indazole, pyrrole, pyrazole, pyridine, pyrimidine, naphthalene, indene, dibenzofuran, benzodioxole, amide, alkyl, aryl, alkyl-aryl, alkyl-heteroaryl, alkenyl, alkenyl-aryl, alkenyl-heteroaryl, peptide, and polypeptide may be optionally substituted with an atom or a group selected from the group consisting of O, N, S, epoxy, —OH, halo, —CN, —NO₂, C₁₋₄alkyl, CF₃, —CF₃O, carbonyl, alkyl-aryl, thiol, methylmethanesulfonamide, methylthiol, dioxane, tetrahydropyzan, morpholinyl, piperazine, sulfonyl, oxazole, oxadiazole, dizaole, triazole, amide, ether, ester and combinations thereof; or R₁ and R₂ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₂ and R₃ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₃ and R₄ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof,
R₆ and R₇ are independently selected from the group consisting of C, N, O and S; or R₆ and R₇ may together form a saturated or unsaturated ring structure that may be optionally substituted with an atom or a group from the group consisting of O, N, halo, C₁₋₄alkyl, CF₃, oxazole, oxadiazole, dizaole, triazole, amide, ether, ester and combinations thereof,
R₈ is selected from the group consisting of H, D, C, O, N, alkenyl, alkynyl, ester, oxazole, piperazine, morpholinyl and halo,
R₉, R₁₀, R₁₁ and R₁₂ are independently selected from the group consisting of no atom, H, D, —OH, halo, —CN, —NO₂, C₁₋₄alkyl, CF₃, —CF₃O, carbonyl, aryl, alkyl-aryl, thiol, ether, and ester,
wherein R is selected from the group consisting of H, D, O, N, halo, oxazole, oxadiazole, dizaole, triazole, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of O, N, S, halo, C₁₋₄alkyl, CF₃, —CN and combinations thereof,
or an N-oxide, crystalline form, hydrate, or pharmaceutically acceptable salt thereof.

Another embodiment of the present disclosure is a compound according to formula (3):

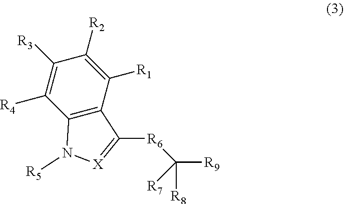

wherein:
X is selected from the group consisting of O, N, S, and C;
R₁, R₂, R₃, R₄, R₅ and R₉ are independently selected from the group consisting of H, D, —OH, halo, ether, ester, furanyl, indole, indazole, pyrrole, pyrazole, pyridine, pyrimidine, naphthalene, indene, dibenzofuran, benzodioxole, amide, —(O)C(R), —C(O)OR, —NO₂, alkyl, aryl, alkyl-aryl, alkyl-heteroaryl, alkenyl, alkenyl-aryl, alkenyl-heteroaryl, peptide, and polypeptide, wherein the ether, ester, furanyl, indole, indazole, pyrrole, pyrazole, pyridine, pyrimidine, naphthalene, indene, dibenzofuran, benzodioxole, amide, alkyl, aryl, alkyl-aryl, alkyl-heteroaryl, alkenyl, alkenyl-aryl, alkenyl-heteroaryl, peptide, and polypeptide may be optionally substituted with an atom or a group selected from the group consisting of O, N, S, epoxy, —OH, halo, —CN, —NO₂, C₁₋₄alkyl, CF₃, —CF₃O, carbonyl, alkyl-aryl, thiol, methylmethanesulfonamide, methylthiol, dioxane, tetrahydropyzan, morpholinyl, piperazine, sulfonyl, oxazole, oxadiazole, dizaole, triazole, amide, ether, ester and combinations thereof; or R₁ and R₂ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₂ and R₃ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₃ and R₄ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof,
R₆ is selected from the group consisting of amide, oxazole, oxadiazole, diazole, and triazole,
R₇ and R₈ are independently selected from the group consisting of no atom, H, D, —OH, halo, —CN, —NO₂, C₁₋₄alkyl, CF₃, —CF₃O, carbonyl, aryl, alkyl-aryl, thiol, ether, and ester,
wherein R is selected from the group consisting of H, D, O, N, halo, oxazole, oxadiazole, dizaole, triazole, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of O, N, S, halo, C₁₋₄alkyl, CF₃, —CN and combinations thereof, or an N-oxide, crystalline form, hydrate, or pharmaceutically acceptable salt thereof.

Another embodiment of the present disclosure is a compound according to formula (4):

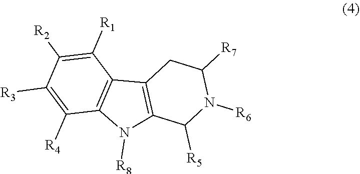

wherein:
R₁, R₂, R₃, R₄, R₅ and R₈ are independently selected from the group consisting of H, D, —OH, halo, ether, ester, furanyl, indole, indazole, pyrrole, pyrazole, pyridine, pyrimidine, naphthalene, indene, dibenzofuran, benzodioxole, amide, —(O)C(R), —C(O)OR, —NO₂, alkyl, aryl, alkyl-aryl, alkyl-heteroaryl, alkenyl, alkenyl-aryl, alkenyl-heteroaryl, peptide, and polypeptide, wherein the ether, ester, furanyl, indole, indazole, pyrrole, pyrazole, pyridine, pyrimidine, naphthalene, indene, dibenzofuran, benzodioxole, amide, alkyl, aryl, alkyl-aryl, alkyl-heteroaryl, alkenyl, alkenyl-aryl, alkenyl-heteroaryl, peptide, and polypeptide may be optionally substituted with an atom or a group selected from the group consisting of O, N, S, epoxy, —OH, halo, —CN, —NO₂, C₁₋₄alkyl, CF₃, —CF₃O, carbonyl, alkyl-aryl, thiol, methylmethanesulfonamide, methylthiol, dioxane, tetrahydropyzan, morpholinyl, piperazine, sulfonyl, oxazole, oxadiazole, dizaole, triazole, amide, ether, ester and combinations thereof; or R₁ and R₂ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₂ and R₃ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₃ and R₄ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof,
R₆ is selected from the group consisting of H, D, —OH, halo, C₁₋₄alkyl, CF₃, —(O)C(R), —C(O)OR, —NO₂, oxazole, oxadiazole, dizaole, triazole, amide, alcohol, ether, ester, and —C(O)C≡CSi(CH₃)₃,
R₇ is selected from the group consisting of H, D, —OH, halo, C₁₋₄alkyl, CF₃, —(O)C(R), —C(O)OR, —NO₂, oxazole, oxadiazole, dizaole, triazole, amide, alcohol, ether, and ester,
wherein R is selected from the group consisting of H, D, O, N, halo, oxazole, oxadiazole, dizaole, triazole, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of O, N, S, halo, C₁₋₄alkyl, CF₃, —CN and combinations thereof,
with the proviso that the compound is not

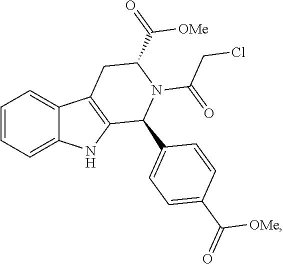 [SMILES: COC(=O)c1ccc([C@H]2c3[nH]c4ccccc4c3C[C@H](C(=O)OC)N2C(=O)CCl)cc1]

or an N-oxide, crystalline form, hydrate, or pharmaceutically acceptable salt thereof.

Another embodiment of the present disclosure is a compound according to formula (5):

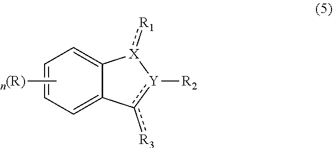

wherein:
a dashed line indicates the presence of an optional double bond;
n=2, 3, or 4;
X and Y are independently selected from the group consisting of C, N, S and O;
R₁, R₂, and R₅ are independently selected from the group consisting of no atom, H, D, O, N, halo, ether, ester, amide, C(O), (O)C(R), C(O) O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of N, O, Sn, halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not

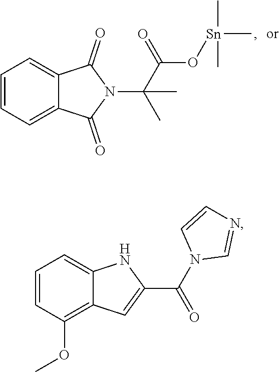 [SMILES: CC(C)(C(=O)[O][Sn]([CH3])([CH3])[CH3])N1C(=O)c2ccccc2C1=O ; COc1cccc2[nH]c(C(=O)n3ccnc3)cc12]

or an N-oxide, crystalline form, hydrate, or pharmaceutically acceptable salt thereof.

Another embodiment of the present disclosure is a composition, including pharmaceutical compositions, comprising one or more compounds disclosed herein and a pharmaceutically acceptable carrier, adjuvant or vehicle.

Another embodiment of the present disclosure is a method for treating or ameliorating the effects of a glutathione peroxidase 4 (GPX4)-associated disease in a subject in need thereof, comprising administering to the subject an effective amount of one or more compounds disclosed herein or the composition disclosed herein.

Another embodiment of the present disclosure is a method for modulating the activity of glutathione peroxidase 4 (GPX4) in a subject in need thereof, comprising administering to the subject an effective amount of one or more compounds disclosed herein or the composition disclosed herein.

Another embodiment of the present disclosure is a method for increasing the level of peroxide in a subject in need thereof, comprising administering to the subject an effective amount of one or more compounds disclosed herein or the composition disclosed herein.

A further embodiment of the present disclosure is a method for inducing ferroptosis in a cell, comprising contacting the cell with an effective amount of one or more compounds disclosed herein or the composition disclosed herein.

Another embodiment of the present disclosure is a method for treating or ameliorating the effects of a cancer in a subject in need thereof, comprising administering to the subject i) an effective amount of a first anti-cancer agent, which is one or more compounds disclosed herein or the composition disclosed herein, and ii) an effective amount of a second anti-cancer agent.

An additional embodiment of the present disclosure is a kit for treating or ameliorating the effects of a glutathione peroxidase 4 (GPX4)-associated disease in a subject in need thereof, comprising an effective amount of one or more compounds disclosed herein or the composition disclosed herein, packaged with its instructions for use.

### BRIEF DESCRIPTION OF THE DRAWINGS

To facilitate further description of the embodiments of this disclosure, the following drawings are provided to illustrate and not to limit the scope of the disclosure.

FIGS. 1A-1B show MST binding traces of LOC1886 (A) and crystal structure of GPX4^{U46C} with LOC1886 (B).

FIGS. 1C-1D show that LOC1886 inhibits and degrades GPX4 (C) and induces lipid peroxidation in cells (D).

FIG. 2 is a schematic of assay funnel for candidate validation.

FIG. 3A shows observed K_{d} for QW-314, a LOC1886 analog, measured by MST. Enhancement on binding affinity was reported, which is comparable to that of RSL3 and significantly improved from LOC1886.

FIGS. 3B-3C show that QW-314 has a high selectivity for GPX4 over GPX1 (B) and induces GPX4 protein degradation (C).

FIG. 4 shows lipid peroxidation flow cytometry assay of RSL3 and QW-314.

FIG. 5 shows cellular dose response assays of QW-314 with HT1080, HLF, HepG2, Huh7 and Skhep-1 liver cancer cells.

FIG. 6 shows that QW-314 has ˜1 μM micromolar potency in Huh7 and HLF cell lines, and is approximately 10 times less potent than RSL3 in all cell lines tested.

FIGS. 7A-7C show the test of QW-314 using a newly developed non-small cell lung carcinoma (NSCLC) model of drug tolerant persister (DTP) cells. FIG. 7A shows that compared to the parental PC9 cells, DTPs are CD133 and C24 positive. FIG. 7B shows that DTPs are specifically sensitive to GPX4 inhibitors. FIG. 7C shows that QW-314 exhibited selective lethality in DTPs vs PC9 parental cells in a dose dependent manner, which was rescued by fer-1.

FIGS. 8A-8B show the in vitro inhibitory efficiencies of selected LOC1886 analogs using NADPH-coupled GPX4 inhibition assay. FIG. 8A shows that QW-356 has significant improvement on GPX4 inhibition over QW-314 and RSL3. FIG. 8B shows similar effect between QW-369 and QW-380.

FIG. 9 shows representative synthesized QW-314 analogs.

FIG. 10 shows NADPH-coupled GPX4 inhibition assay for representative QW-314 analogs of FIG. 9.

FIG. 11 shows C11-BODIPY lipid peroxidation flow cytometry assay in HT1080 cells for selected QW-314 analogs.

FIG. 12 shows cellular dose response assays of representative QW-314 analogs with HT1080 cells using RSL3 as control. GI₅₀ values of these analogs were reported.

FIG. 13 shows cellular dose response assays of representative QW-314 analogs with SUDHL6 cells using RSL3 as control. GI₅₀ values of these analogs were reported.

FIG. 14 shows observed K_{d} for QW-446, measured by MST. Enhancement on binding affinity was reported.

FIG. 15A shows structures of additional QW-446 derivatives.

FIG. 15B shows that QW-594 has GPX4 inhibitory activity that is comparable to QW-446.

FIG. 15C shows that additional warheads do not confer improved potency.

FIG. 15D shows that water solubilizing groups lead to slightly diminished potency.

FIG. 16A shows additional synthesized QW-446 analogs with different warheads.

FIG. 16B shows that QW-446 remains the most potent analog, and alternative warheads are not as active.

FIG. 17A shows compounds synthesized by replacing amide for ester linkage.

FIG. 17B shows that amide analogs of top leads showing diminished GPX4 inhibitory activity in vitro.

FIG. 17C shows that QW-624 is still active in lipid peroxidation assay in HT-1080 cells.

FIG. 18A shows compounds with novel warheads, water-solubilizing groups and heterocycles.

FIG. 18B shows that QW-655 has the greatest potency.

FIG. 19 shows sites of metabolism predicting metabolic liabilities in QW series compounds.

FIG. 20A shows LOC1886 analogs with amide bond replaced by triazole.

FIG. 20B shows that replacement of the amide bond with triazole is tolerated.

FIG. 21A shows LOC1886 analogs synthesized by reversing the amide bond in the linker and introduction of cyclic linker and —CF₃ group.

FIG. 21B shows that reversing the amide bond in the linker and introduction of cyclic linker and —CF₃ group resulted in loss of GPX4 inhibitory activity in vitro.

FIG. 22A shows cellular dose response assays of representative QW-655 analogs with HT1080 cells using RSL3 as control. GI₅₀ values of these analogs were reported.

FIG. 22B shows cellular dose response assays of representative QW-671 analogs with HT1080 cells using RSL3 as control. GI₅₀ values of these analogs were reported.

FIG. 23A shows LOC1886 analogs synthesized by reverse of the amide bond and introduction of novel water-solubilizing groups and heterocycles.

FIG. 23B shows NADPH-coupled GPX4 inhibition assay for representative analogs of FIG. 23A.

FIG. 23C shows cellular dose response assays of representative QW-711 and QW-712 analogs with HT1080 cells using RSL3 as control. GI₅₀ values of these analogs were reported.

FIG. 23D shows cellular dose response assays of representative QW-715 and QW-716 analogs with HT1080 cells using RSL3 as control. GI₅₀ values of these analogs were reported.

FIG. 24A shows representative synthesized LOC1886 analogs with secondary and tertiary alcohols in the linker.

FIG. 24B shows NADPH-coupled GPX4 inhibition assay for representative analogs of FIG. 24A.

FIG. 24C shows cellular dose response assays of representative QW-730 and QW-736 analogs with HT1080 cells using RSL3 as control. GI₅₀ values of these analogs were reported.

FIG. 25A shows structures of analogs with diverse linkers and heterocycles.

FIG. 25B shows structures of analogs with modifications at different sites.

FIG. 25C shows cellular dose response assays of representative QW-744, QW-750 and QW-755 analogs with HT1080 cells using RSL3 as control. GI₅₀ values of these analogs were reported.

FIG. 25D shows cellular dose response assays of representative QW-731 and QW-774 analogs with HT1080 cells using RSL3 as control. GI₅₀ values of these analogs were reported.

FIG. 25E shows cellular dose response assays of representative QW-766, QW-770 and QW-786 analogs with HT1080 cells using RSL3 as control. GI₅₀ values of these analogs were reported.

FIG. 25F shows cellular dose response assays of representative QW-796 and QW-809 analogs with HT1080 cells using RSL3 as control. GI₅₀ values of these analogs were reported.

FIG. 25G shows cellular dose response assays of representative QW-671, QW-792 and QW-801 analogs with HT1080 cells using RSL3 as control. GI₅₀ values of these analogs were reported.

FIG. 25H shows cellular dose response assays of representative QW-813 and QW-815 analogs with HT1080 cells using RSL3 as control. GI₅₀ values of these analogs were reported.

FIG. 26A shows structures of additional analogs.

FIG. 26B shows NADPH-coupled GPX4 inhibition assay for representative analogs of FIG. 26A.

FIG. 26C shows cellular dose response assays of representative QW-823 and QW-824 analogs with HT1080 cells using RSL3 as control. GI₅₀ values of these analogs were reported.

FIG. 26D shows that QW-823 and QW-824 lost potency at lower concentration.

FIG. 26E is a schematic showing the docking of QW-823 and QW-824 onto wildtype GPX4.

FIG. 27A shows structures of some masked propiolamide RSL3 analogs.

FIG. 27B shows that protecting group trimethylsilyl (TMS) leaves before masked propiolamide analogs of RSL3 bind to GPX4.

FIG. 27C shows NADPH-coupled GPX4 inhibition assay for representative QW-314 analogs of FIG. 27A.

FIG. 27D shows that masked propiolamide analogs of RSL3 induced ferroptosis in HT-1080 cells.

FIG. 27E shows C11-BODIPY lipid peroxidation flow cytometry assay in HT1080 cells for selected RSL3 analogs.

FIG. 28 shows that analogs VP-21, VP-34 and VP-73 are more potent than RSL3 in the cellular dose response assays.

FIG. 29 shows that VP-34 induced ferroptosis in SU-DHL-6 B cell lymphoma cells with 30× greater potency than RSL3.

FIG. 30 is a schematic showing the docking of VP-34 onto double mutant GPX4.

FIG. 31A shows modifications on VP-34 employing Suzuki coupling.

FIG. 31B shows structures of compounds synthesized following the scheme in FIG. 31A.

FIG. 31C shows NADPH-coupled GPX4 inhibition assay for representative analogs of FIG. 31B.

FIG. 32A shows structures of masked propiolamide analogs of RSL3.

FIG. 32B shows NADPH-coupled GPX4 inhibition assay for representative analogs of FIG. 32A.

FIG. 33A shows VP-34 derivatives synthesized by adding extra —Br group while keeping the —BR on the indole ring intact.

FIG. 33B shows NADPH-coupled GPX4 inhibition assay for representative analogs of FIG. 33A.

FIG. 34A shows structures of additional RSL3 analogs.

FIG. 34B shows NADPH-coupled GPX4 inhibition assay for representative analogs of FIG. 34A.

FIG. 35A shows modifications on VP-171 employing (CuAAC) click chemistry.

FIG. 35B shows NADPH-coupled GPX4 inhibition assay for representative analogs such as VP-180.

FIG. 35C shows NADPH-coupled GPX4 inhibition assay for representative analogs including VP-239 and VP-256.

FIG. 35D shows that both VP-171 and VP-180 induced fer-1 rescuable lipid peroxidation.

FIG. 36 shows the solubility results of representative LOC1886 and RSL3 analogs.

FIG. 37 shows the ADMET analysis of selected analogs specifically their plasma stability assay results.

FIG. 38A shows modifications on VP-34 employing Suzuki coupling.

FIG. 38B shows modifications on VP210 by replacing the benzene-ring with aza-arenes.

FIG. 38C shows schemes for further modifications.

FIG. 38D shows structures of compounds synthesized by single-Suzuki coupling and double-Suzuki coupling.

FIG. 38E shows NADPH-coupled GPX4 inhibition assay for representative analogs of FIG. 38D.

FIG. 39A shows VP-224 analogs with addition of hetero-atom and —CF₃ group.

FIG. 39B shows NADPH-coupled GPX4 inhibition assay for representative analogs of FIG. 39A.

FIG. 40A structures of VP-224 analogs with small modifications on the phenyl rings.

FIG. 40B shows NADPH-coupled GPX4 inhibition assay for representative analogs of FIG. 40A.

FIG. 41 shows cellular dose response assays of representative VP-224 analogs with HT1080 cells using RSL3 as control. GI₅₀ values of these analogs were reported.

FIG. 42 shows cellular dose response assays of representative VP series analogs with HT1080 cells using RSL3 as control. GI₅₀ values of these analogs were reported.

FIG. 43 shows cellular dose response assays of representative VP-288 and VP-297 analogs with HT1080 cells using RSL3 as control. GI₅₀ values of these analogs were reported.

FIG. 44 shows cellular dose response assays of representative VP-304 and VP-306 analogs with HT1080 cells using RSL3 as control. GI₅₀ values of these analogs were reported.

FIG. 45A shows microsomal intrinsic clearance assay results of selected analogs.

FIG. 45B shows plasma stability assay results of selected analogs.

FIG. 46A shows microsomal stability results of additional analogs.

FIG. 46B shows plasma stability assay results of additional analogs.

FIG. 47 shows suggested metabolic pathway for VP-171 after incubations with human hepatocytes.

FIG. 48 shows sites of metabolism predicting metabolic liabilities in VP-171.

FIG. 49A shows analogs synthesized by replacing the —CO₂Me group.

FIG. 49B NADPH-coupled GPX4 inhibition assay for representative analogs of FIG. 49A.

FIG. 50 shows that VP-253 had similar GPX4 inhibition as VP-171 at 1 μM.

FIG. 51 shows that removal of —Br and methyl ester groups leads to loss of GPX4 inhibitory activity.

FIG. 52 shows analogs with and without —Br and/or methyl ester as well as with different halogens.

FIG. 53 shows cellular dose response assays of VP-328 with HT1080 cells using RSL3 as control. GI₅₀ values of these analogs were reported.

FIG. 54 shows cellular dose response assays of representative VP-330 and VP-334 analogs with HT1080 cells using RSL3 as control. GI₅₀ values of these analogs were reported.

FIG. 55 shows cellular dose response assays of masked and unmasked VP343 with HT1080 cells using RSL3 as control. GI₅₀ values of these analogs were reported.

FIG. 56 shows cellular dose response assays of representative VP-358 and VP-360 analogs with HT1080 cells using RSL3 as control. GI₅₀ values of these analogs were reported.

FIG. 57 shows that VP-328 is as selective and slightly more potent than VP-224, but is not rescued by fer-1 at same concentrations as VP-224 in DTPs.

FIG. 58 shows that halogen substitution (VP-360) has minor effect on potency in DTPs.

FIG. 59 shows that VP-328 is rescued by fer-1 at lower concentration (1 μM) in DTPs (and is rescued in HT1080s).

FIG. 60 shows that DTPs are more susceptible to rescue by fer-1 than HT1080s from VP-224 induced ferroptosis.

FIG. 61 shows that VP-343 (both masked and unmasked) lost strong selectivity for DTPs, and VP343m lost potency in DTPs.

FIG. 62 shows that VP-343 (both masked and unmasked) are not rescued by fer-1 in DTPs or HT1080s.

FIG. 63 shows that halogen presence and type has substantial (˜50%) effect on fer-1 ability to rescue, and halogen presence alone has moderate (˜25%) effect on potency in DTPs.

FIG. 64 shows that VP-358 and VP-360 are both selective for DTPs and rescued by fer-1.

FIG. 65 shows that VP-358 and VP-360 rescue is not consistent between DTPs and HT1080s.

FIG. 66 shows that in absence of halogen, Ester is sufficient, but not necessary, for ferroptosis in DTPs; in presence of halogen, Ester is necessary for ferroptosis.

FIG. 67 shows that ester and halogen combinations modulate ferroptosis differently in different cell types.

FIG. 68 shows ADMET analysis of selected LOC1886 analogs.

FIG. 69 shows ADMET analysis of QW-316 and VP-72.

FIG. 70 shows ADMET analysis of selected LOC1886 and RSL3 analogs.

FIG. 71 shows microsomal stability results of selected LOC1886 and RSL3 analogs.

FIG. 72 shows plasma stability assay results of selected LOC1886 and RSL3 analogs.

FIG. 73 shows solubility results of some lead LOC1886 and RSL3 analogs.

FIG. 74 shows formulation for in vivo pharmacokinetic study. VP-224 and QW-594 were tested in vivo for mouse plasma and tumor stability, and compared to RSL3. SHDHL-6 (large-cell lymphoma) cells were injected to mice and allowed to form tumors for 2.5 weeks prior to compound administration. Modes of administration: IP, PO. Plasma and tumor samples were collected at 0.5, 1, 2, 4, 8 and 24 hours after compound administration. Compound levels were measured by LC/MS.

FIG. 75A shows that VP-224, but not QW-594, is both selective and lethal in DTPs vs PC9s.

FIG. 75B shows that VP-224 is both selective and lethal in DTPs vs PC9s at >500 nM and is substantially rescued by Fer-1.

FIG. 75C shows that VP-224 and QW-594 both induce ferroptosis in SU-DHL-6 B cell lymphoma cells.

FIG. 76 shows the in vivo pharmacokinetic study results for VP-224.

FIG. 77 shows the in vivo pharmacokinetic study results, in which unmasked version of VP-224 was detected in plasma and tumor samples.

FIG. 78 shows the in vivo pharmacokinetic study results for QW-594.

FIG. 79 shows that both LOC1886 and RSL3 scaffolds show specificity for DTP vs PC9 cells.

FIG. 80A shows that QW-446 and VP-171, but not VP-180, show DTP selectivity relative to PC9 parental cells.

FIG. 80B shows that Fer-1 rescues DTPs from QW-446 and VP-171.

FIGS. 81A-81F show that LOC1886 is a hit compound and has low potencies against GPX4.

FIG. 81A shows the chemical structure of LOC1886.

FIG. 81B shows the cellular dose-response curves of HT-1080 cells treated with LOC1886 with and without fer-1.

FIG. 81C shows the in vitro GPX4 activity assay of 200 μM LOC1886. Data are plotted as mean±SD, n=3.

FIG. 81D shows flow cytometry with C11-BODIPY assessment of lipid peroxidation induced by treatment of HT-1080 cells with 100 μM QW-057 for two hours with and without fer-1.

FIG. 81E shows the cellular dose-response curves of HT-1080 cells treated with RSL3, QW-057 with and without fer-1.

FIG. 81F shows the molecular modeling of LOC1886 bound at the active site of wildtype GPX4.

FIGS. 82A-82G show that QW-156 is most potent LOC1886 analog in generation 1.

FIG. 82A shows the scaffold hopping strategy.

FIG. 82B shows the molecular modeling of QW-144 bound at the active site of wildtype GPX4.

FIG. 82C shows the structure of QW-156.

FIG. 82D shows the cellular dose-response curves of QW-156 in three cancer cell lines.

FIG. 82E shows the dose titration of QW-152, QW-156, and QW-158 in in vitro GPX4 activity assay. RSL3 is included as a positive control.

FIG. 82F shows flow cytometry with C11-BODIPY assessment of lipid peroxidation induced by treatment of HT-1080 cells with 150 nM RSL3, 10 μM QW-156 for two hours with and without fer-1.

FIG. 82G shows QW-156's selectivity to GPX4.

FIGS. 83A-83F show Representative GPX4 inhibitor QW-314 in generation 2.

FIG. 83A shows the structure of QW-314.

FIG. 83B shows that QW-314 shows greater GPX4 inhibitory activity than RSL3 and selectivity for GPX4 over GPX1.

FIG. 83C shows that QW-314 induces ferrostatin-rescuable lipid peroxidation.

FIG. 83D shows that QW-314 shows hallmarks of GPX4 inhibition in 5 tumor cell lines.

FIG. 83E shows that QW-314 induces GPX4 protein degradation.

FIG. 83F shows the cocrystal of QW-314 with GPX4^{U46C}.

FIGS. 84A-84F show representative GPX4 inhibitor QW-446 in generation 3.

FIG. 84A shows the structure of QW-446.

FIG. 84B shows that QW-446 shows greater GPX4 inhibitory activity than QW-314 at 5 μM.

FIG. 84C shows that QW-446 induces ferroptosis in HT-1080 cells with GI₅₀s in the nanomolar range.

FIG. 84D shows that QW-446 induces ferrostatin-rescuable lipid peroxidation.

FIG. 84E shows QW-446, QW-314 and RSL3 IC₅₀ values as determined in the in vitro GPX4 activity assay. Data are plotted as mean±SD, n=3.

FIG. 84F shows that QW-446 docks well in the absence of intermolecular interaction.

FIGS. 85A-85D show Representative GPX4 inhibitor QW-852 in generation 4.

FIG. 85A shows the structures of QW-851, QW-852 and QW-857.

FIG. 85B shows that QW-851, QW-852 and QW-857 shows greater GPX4 inhibitory activity than QW-446 at 2.5 μM concentration.

FIG. 85C shows potency and ferroptosis selectivity of RSL3, QW-851, QW-852 and QW-857 in HT-1080 cells.

FIG. 85D shows cell viability of RSL3 dose at 8 μM, QW-851, QW-852 and QW-857 dose at 10 μM in HT1080 cells.

FIG. 86 shows structures of selected LOC1886 analogs.

FIG. 87 shows structures of series 1 analogs in generation 1.

FIG. 88 shows structures of series 2 analogs in generation 1.

FIG. 89 shows structures of series 1 analogs in generation 2.

FIG. 90 shows structures of series 2 analogs in generation 2.

FIG. 91 shows structures of series 3 analogs in generation 2.

FIG. 92 shows structures of series 1 analogs in generation 3.

FIG. 93 shows structures of series 2 analogs in generation 3.

FIG. 94 shows structures of series 3 analogs in generation 3.

FIG. 95 shows structures of series 4 analogs in generation 3.

FIG. 96 shows pharmacokinetic study of compound QW-594 and RSL3.

FIG. 97 shows structures of series 1 analogs in generation 4.

FIG. 98 shows structures of series 2 analogs in generation 4.

FIG. 99 shows structures of series 3 analogs in generation 4.

FIG. 100 shows structures of series 4 analogs in generation 4.

FIGS. 101A-101B show that VP-224 and QW-852 induce ferroptosis in cancer cells.

FIG. 101A shows the dose-response curves of HT-1080 and SU-DHL-6 cells treated with VP-224 and QW-852, with or without cell death inhibitors, Fer-1 (1.0 μM), Lip-1 (1.0 μM), Z-VAD-FMK (20 μM) and Nec-1s (20 μM) for 48 h and 24 h, respectively. Data are presented as mean±SEM of n=3 technical replicates.

FIG. 101B shows the lipid peroxidation measured by C11-BODIPY^{581/591} in HT-1080 cells treated with GPX4 inhibitors (RSL3, QW-852 and VP-224; 0.15 μM) and with or without Fer-1 (10 μM) for 1 h.

FIG. 102 shows the dose-response curves of compounds 1 (VP-224) and 2 (QW-852), with or without Fer-1 (1.0 μM) in different cancer cell lines. Cells were treated with the corresponding compounds for 24 h.

FIGS. 103A-103E show that VP-224 and QW-852 are covalent GPX4 inhibitors.

FIGS. 103A and 103B show the first-posed docking of the covalent inhibitors VP-224 (A) and QW-852 (B) onto the crystal structure of the wild-type GPX4^{U46}. The wild-type GPX4 (PDB code: 6HKQ) is depicted as electrostatic surface using APBS Electrostatics Plugin (pymolwiki.org/index.php/APBS_Electrostatics_Plugin) in PyMOL (pymol.org/). Important residues that have polar and non-polar interactions with the inhibitors, VP-224 and QW-852 are shown as stick models and labeled. Hydrogen bonds are shown as dash lines.

FIG. 103C shows the proposed mechanism of action of VP-224 and QW-852 with Sec46 of GPX4^{WT}. The postulated product of the covalent reaction with Sec46 of GPX4^{WT} is shown on the right. Red, trimethylsilyl propiolamide warhead; TMS: trimethylsilyl group.

FIG. 103D shows the intact protein MALDI MS of GPX4^{U46C} pre-incubated with DMSO, RSL3, VP-224 or QW-852 (150 μM).

FIG. 103E shows the GPX4 CETSA profiles of intact HT-1080 cells treated with DMSO (orange) and VP-224 (brown) (left) or DMSO (orange) and QW-852 (purple) (right). Cells were treated with 10 μM compound for 1 h. Data are plotted as mean±SEM, n=3 or 4 biologically independent samples.

FIGS. 104A-104C show (A) Second-posed, (B) third-posed and (C) fourth-posed docking of the covalent inhibitor VP-224 onto the crystal structure of the wild-type GPX4^{U46}. The wild-type GPX4 (PDB code: 6HKQ) is depicted as electrostatic surface. Important residues that have polar and non-polar interactions with VP-224 are shown as stick models and labeled. Hydrogen bonds are shown as dash lines.

FIG. 104D shows the GPX1 CETSA profiles of intact HT-1080 cells treated with DMSO and VP-224 or DMSO and QW-852. Cells were treated with 10 μM compound for 1h. Data are plotted as mean±SEM, n=3 biologically independent samples.

FIGS. 105A-105E show the degradation mechanism studies of VP-224 and QW-852.

FIG. 105A shows that GFP-GPX4^{WT} in HT-1080 cells overexpressing GFP-GPX4^{WT} was tested for vulnerability to degradation induced by RSL3, VP-224 and QW-852. Data are presented as mean±SEM, n=2 biologically independent samples.

FIG. 105B shows that GFP-GPX4^{K125R_K127R} in HT-1080 cells overexpressing GFP-GPX4^{K125R_K127R} was tested for vulnerability to degradation induced by RSL3, VP-224 and QW-852. Data are presented as mean±SEM, n=2 biologically independent samples.

FIGS. 105C and 105D show that proteasome inhibitor, MG-132 was tested for its ability to block the degradation of endogenous GPX4 (C) and GFP-GPX4 (D) in HT-1080 cells overexpressing GFP-GPX4^{WT}. Cells were treated with DMSO (control), RSL3 (1.0 μM), VP-224 (1.0 μM), or QW-852 (1.0 μM)±MG-132 (10 μM) for 18 hours. Data are presented as mean±SEM, n=2 biologically independent samples.

FIG. 105E shows that endogenous GPX1 in HT-1080 cells overexpressing GFP-GPX4^{WT} was tested for vulnerability to degradation induced by RSL3, VP-224 and QW-852. Data are presented as mean±SEM, n=2 biologically independent samples.

FIGS. 106A-106E show the in vitro and in vivo safety of VP-224 and QW-852.

FIG. 106A shows the dose-response curves of peripheral blood mononuclear cells (PBMCs) treated with VP-224, QW-852 or RSL3 for 24 h. Data are presented as mean±SEM of n=6 technical replicates.

FIG. 106B shows the body weight changes in male (M) and female (F) C57BL/6 mice following daily intraperitoneal (IP) injections of 20 mg/kg VP-224, QW-852, RSL3 or vehicle (control) for 5 consecutive days. Data are presented as mean±SEM of n=3 biological replicates.

FIG. 106C shows the representative images showing H&E staining of the major organs of male (M) and female (F) C57BL/6 mice upon treatment with 20 mg/kg VP-224, QW-852, RSL3 or vehicle (control), bar=50 μm.

FIG. 106D shows the hepatorenal function evaluation in male (M) and female (F) C57BL/6 mice upon daily treatment with 20 mg/kg VP-224 or vehicle (control) via intraperitoneal (IP) injection for 5 consecutive days. Data are presented as mean±SEM of n=3 biological replicates. One-way ANOVA followed by Šidák's multiple comparisons test was performed: not significant (ns).

FIG. 106E shows the final concentration of VP-224 in the brain tissue of C57BL/6 mice upon daily treatment with 20 mg/kg VP-224 via intraperitoneal (IP) injection for 5 consecutive days. Data are presented as mean±SEM of n=3 biological replicates. Unpaired t test was performed: not significant (ns).

FIGS. 107A-107B show the representative western blots and GPX4 protein levels in (A) liver and (B) kidney tissue of healthy C57BL/6 mice upon daily treatment with 20 mg/kg VP-224 or vehicle (control) via intraperitoneal (IP) injection for 5 consecutive days. Data are plotted as mean±SEM, n=3 biologically independent samples. One-way ANOVA followed by Šidák's multiple comparisons test was performed: not significant (ns).

### DETAILED DESCRIPTION OF THE DISCLOSURE

In the present disclosure, improved analogs of identified GPX4 binders were created and tested. The output of these studies serves as a starting lead that can be evaluated later for PK/PD, safety, and selectivity. In parallel, the screening assay is optimized, and triage compounds that emerge from the screen are evaluated.

Structure-Activity Relationship (SAR) optimizations on LOC1886 were conducted. Several analogs of LOC1886 were identified with better binding affinity to GPX4, according to MST (Microscale Thermophoresis) tests and their structures provided us with novel pharmacophores for further optimization. MST measurement for the remaining analogs and detailed biophysical and biochemical characterizations are currently tested. GPX4-U46C was crystallized in complex with LOC1886. The crystals are small and diffracted X-ray at APS beam line NE_24ID_C poorly (˜4 angstrom).

Accordingly, one embodiment of the present disclosure is a compound according to formula (1):

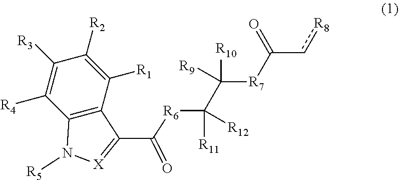

wherein:
a dashed line indicates the presence of an optional double bond;
X is selected from the group consisting of O, N, S, and C;
R₁, R₂, R₃, R₄ and R₅ are independently selected from the group consisting of H, D, —OH, halo, ether, ester, furanyl, indole, indazole, pyrrole, pyrazole, pyridine, pyrimidine, naphthalene, indene, dibenzofuran, benzodioxole, amide, —(O)C(R), —C(O)OR, —NO₂, alkyl, aryl, alkyl-aryl, alkyl-heteroaryl, alkenyl, alkenyl-aryl, alkenyl-heteroaryl, peptide, and polypeptide, wherein the ether, ester, furanyl, indole, indazole, pyrrole, pyrazole, pyridine, pyrimidine, naphthalene, indene, dibenzofuran, benzodioxole, amide, alkyl, aryl, alkyl-aryl, alkyl-heteroaryl, alkenyl, alkenyl-aryl, alkenyl-heteroaryl, peptide, and polypeptide may be optionally substituted with an atom or a group selected from the group consisting of O, N, S, epoxy, —OH, halo, —CN, —NO₂, C₁₋₄alkyl, CF₃, —CF₃O, carbonyl, alkyl-aryl, thiol, methylmethanesulfonamide, methylthiol, dioxane, tetrahydropyzan, morpholinyl, piperazine, sulfonyl, oxazole, oxadiazole, dizaole, triazole, amide, ether, ester and combinations thereof; or R₁ and R₂ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₂ and R₃ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₃ and R₄ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof,
R₆ and R₇ are independently selected from the group consisting of C, N, O and S; or R₆ and R₇ may together form a saturated or unsaturated ring structure that may be optionally substituted with an atom or a group from the group consisting of O, N, halo, C₁₋₄alkyl, CF₃, oxazole, oxadiazole, dizaole, triazole, amide, ether, ester and combinations thereof,
R₈ is selected from the group consisting of H, D, C, O, N, alkenyl, alkynyl, ester, oxazole, piperazine, morpholinyl and halo,
R₉, R₁₀, R₁₁ and R₁₂ are independently selected from the group consisting of no atom, H, D, —OH, halo, —CN, —NO₂, C₁₋₄alkyl, CF₃, —CF₃O, carbonyl, aryl, alkyl-aryl, thiol, ether, and ester,
wherein R is selected from the group consisting of H, D, O, N, halo, oxazole, oxadiazole, dizaole, triazole, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of O, N, S, halo, C₁₋₄alkyl, CF₃, —CN and combinations thereof,
or an N-oxide, crystalline form, hydrate, or pharmaceutically acceptable salt thereof.

In some embodiments, the compound has a structure selected from the group consisting of:

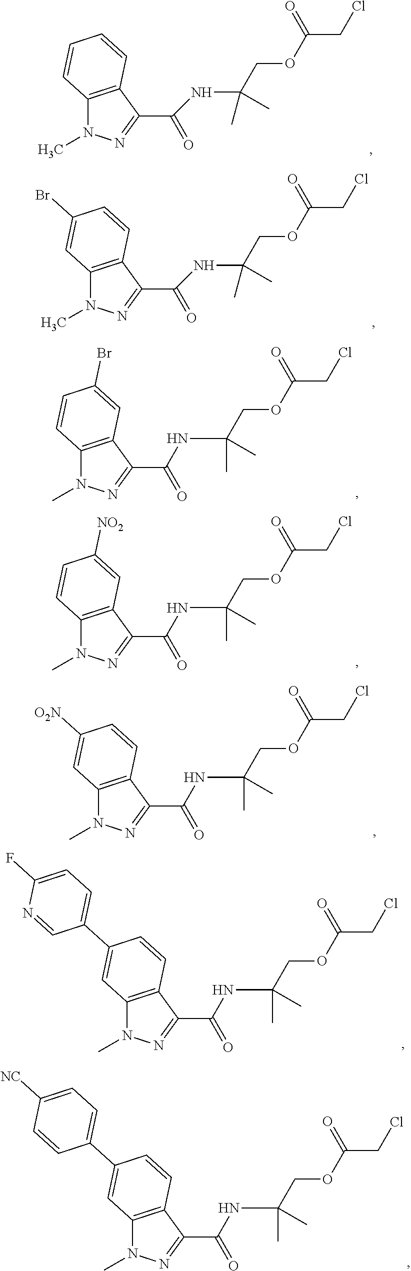

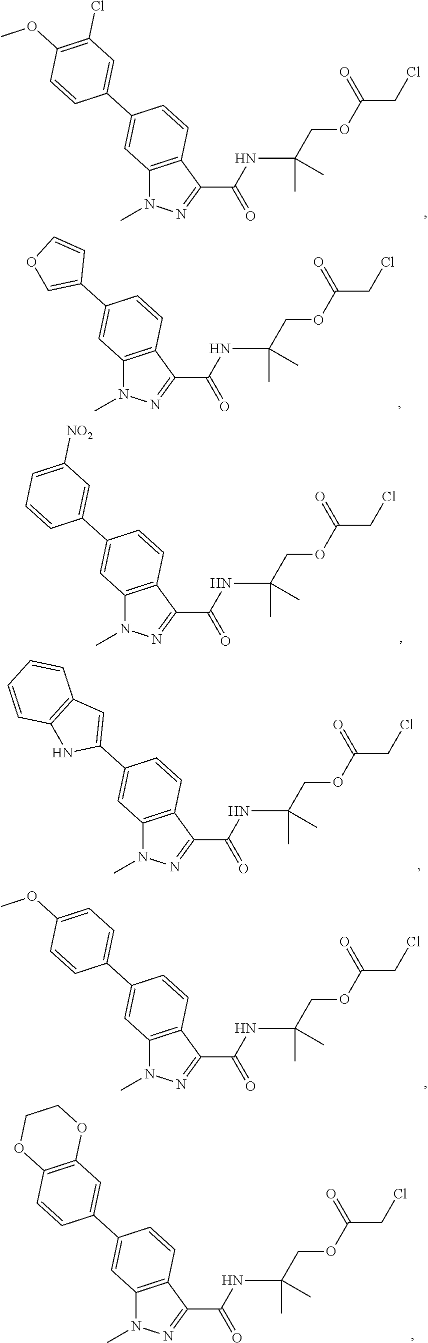

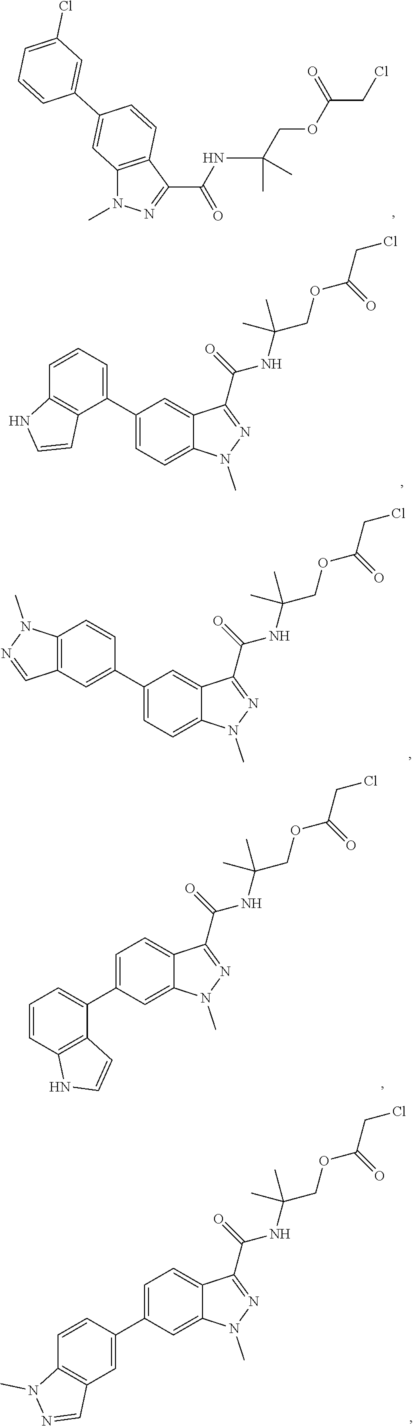 [SMILES: Cn1nc(C(=O)NC(C)(C)COC(=O)CCl)c2ccc(-c3cccc(Cl)c3)cc21 ; Cn1nc(C(=O)NC(C)(C)COC(=O)CCl)c2cc(-c3cccc4[nH]ccc34)ccc21 ; Cn1ncc2cc(-c3ccc4c(c3)c(C(=O)NC(C)(C)COC(=O)CCl)nn4C)ccc21 ; Cn1nc(C(=O)NC(C)(C)COC(=O)CCl)c2ccc(-c3cccc4[nH]ccc34)cc21 ; Cn1ncc2cc(-c3ccc4c(C(=O)NC(C)(C)COC(=O)CCl)nn(C)c4c3)ccc21]

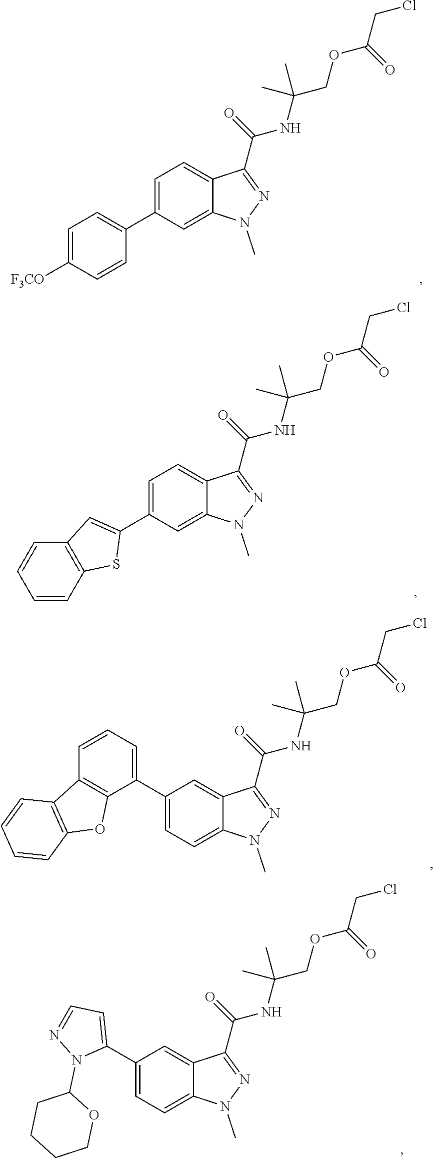 [SMILES: Cn1nc(C(=O)NC(C)(C)COC(=O)CCl)c2ccc(-c3ccc(OC(F)(F)F)cc3)cc21 ; Cn1nc(C(=O)NC(C)(C)COC(=O)CCl)c2ccc(-c3cc4ccccc4s3)cc21 ; Cn1nc(C(=O)NC(C)(C)COC(=O)CCl)c2cc(-c3cccc4c3oc3ccccc34)ccc21 ; Cn1nc(C(=O)NC(C)(C)COC(=O)CCl)c2cc(-c3ccnn3C3CCCCO3)ccc21]

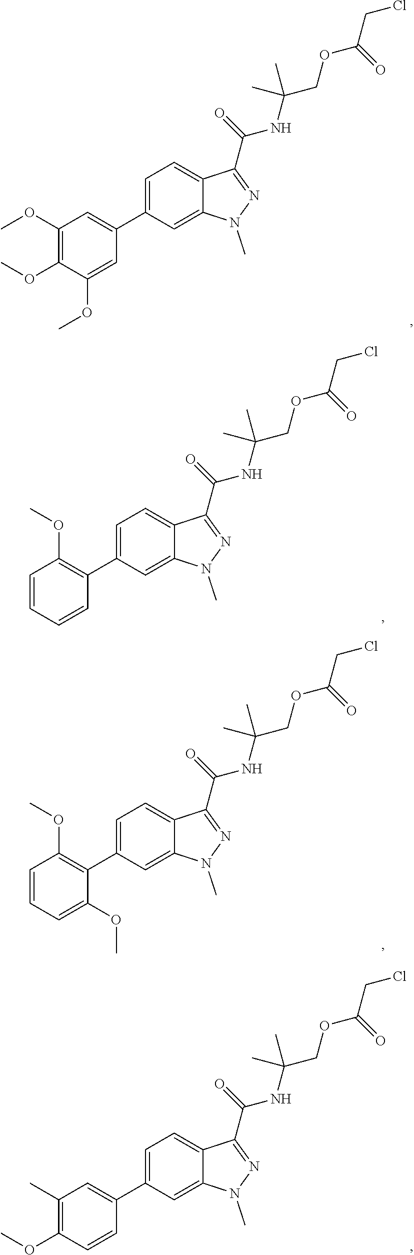 [SMILES: COc1cc(-c2ccc3c(C(=O)NC(C)(C)COC(=O)CCl)nn(C)c3c2)cc(OC)c1OC ; COc1ccccc1-c1ccc2c(C(=O)NC(C)(C)COC(=O)CCl)nn(C)c2c1 ; COc1cccc(OC)c1-c1ccc2c(C(=O)NC(C)(C)COC(=O)CCl)nn(C)c2c1 ; COc1ccc(-c2ccc3c(C(=O)NC(C)(C)COC(=O)CCl)nn(C)c3c2)cc1C]

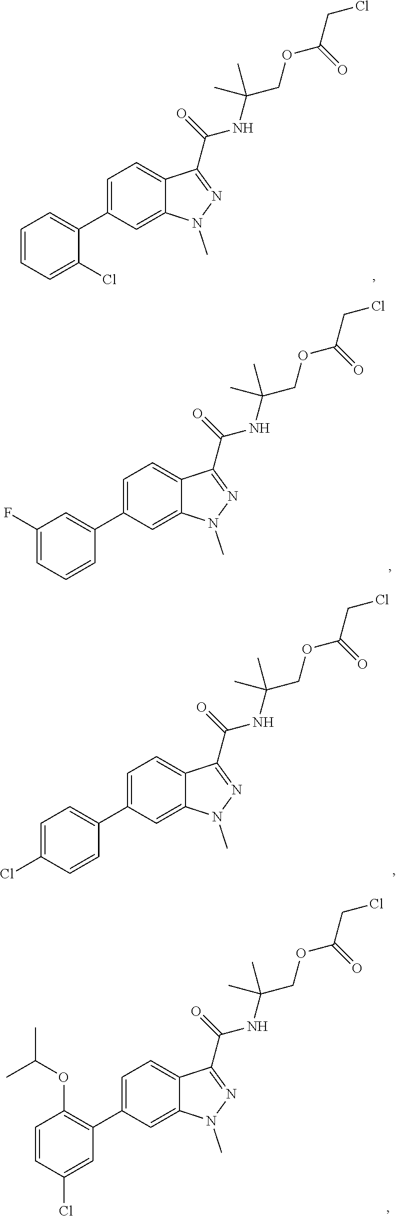 [SMILES: Cn1nc(C(=O)NC(C)(C)COC(=O)CCl)c2ccc(-c3ccccc3Cl)cc21 ; Cn1nc(C(=O)NC(C)(C)COC(=O)CCl)c2ccc(-c3cccc(F)c3)cc21 ; Cn1nc(C(=O)NC(C)(C)COC(=O)CCl)c2ccc(-c3ccc(Cl)cc3)cc21 ; CC(C)Oc1ccc(Cl)cc1-c1ccc2c(C(=O)NC(C)(C)COC(=O)CCl)nn(C)c2c1]

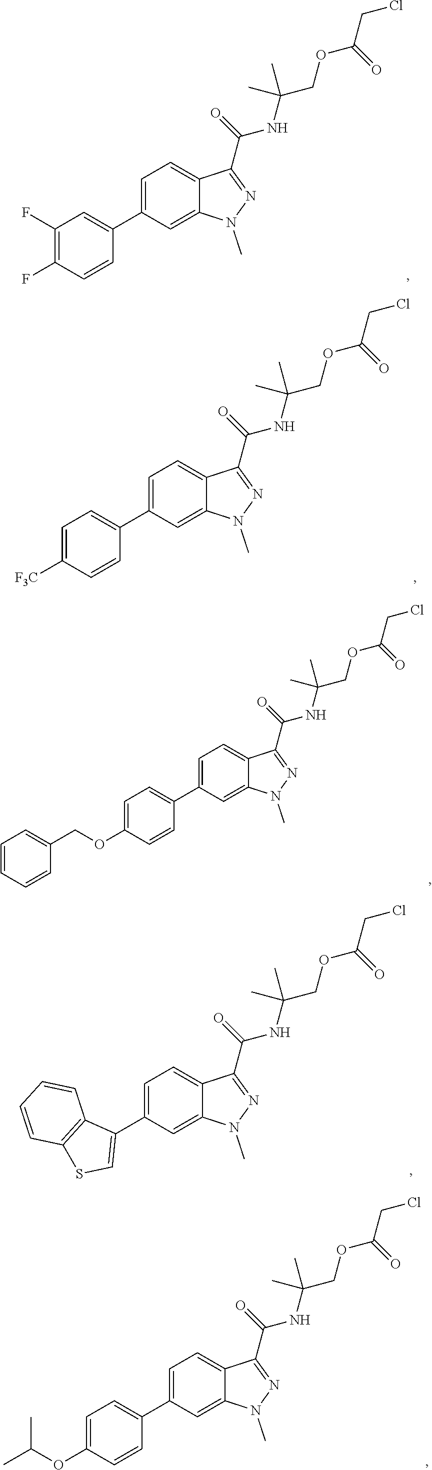 [SMILES: Cn1nc(C(=O)NC(C)(C)COC(=O)CCl)c2ccc(-c3ccc(F)c(F)c3)cc21 ; Cn1nc(C(=O)NC(C)(C)COC(=O)CCl)c2ccc(-c3ccc(C(F)(F)F)cc3)cc21 ; Cn1nc(C(=O)NC(C)(C)COC(=O)CCl)c2ccc(-c3ccc(OCc4ccccc4)cc3)cc21 ; Cn1nc(C(=O)NC(C)(C)COC(=O)CCl)c2ccc(-c3csc4ccccc34)cc21 ; CC(C)Oc1ccc(-c2ccc3c(C(=O)NC(C)(C)COC(=O)CCl)nn(C)c3c2)cc1]

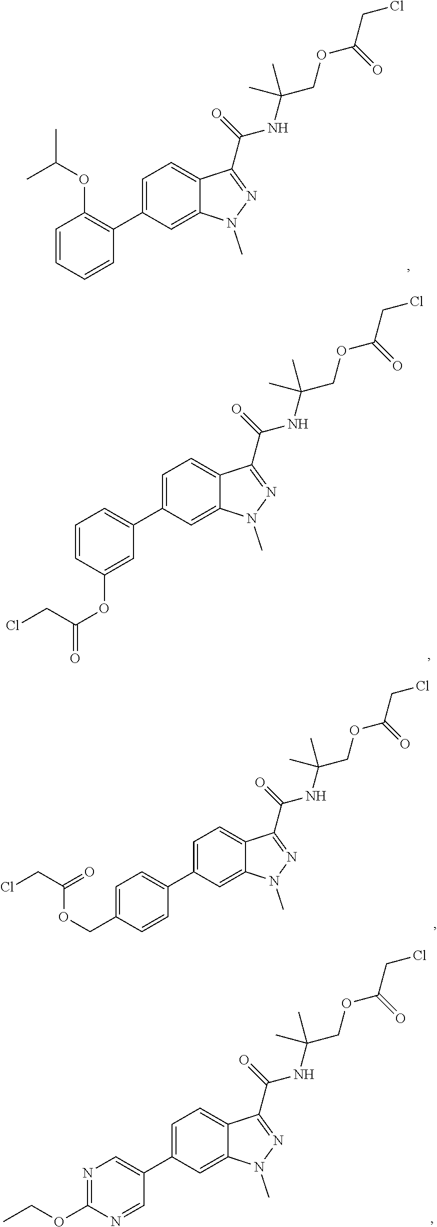 [SMILES: CC(C)Oc1ccccc1-c1ccc2c(C(=O)NC(C)(C)COC(=O)CCl)nn(C)c2c1 ; Cn1nc(C(=O)NC(C)(C)COC(=O)CCl)c2ccc(-c3cccc(OC(=O)CCl)c3)cc21 ; Cn1nc(C(=O)NC(C)(C)COC(=O)CCl)c2ccc(-c3ccc(COC(=O)CCl)cc3)cc21 ; CCOc1ncc(-c2ccc3c(C(=O)NC(C)(C)COC(=O)CCl)nn(C)c3c2)cn1]

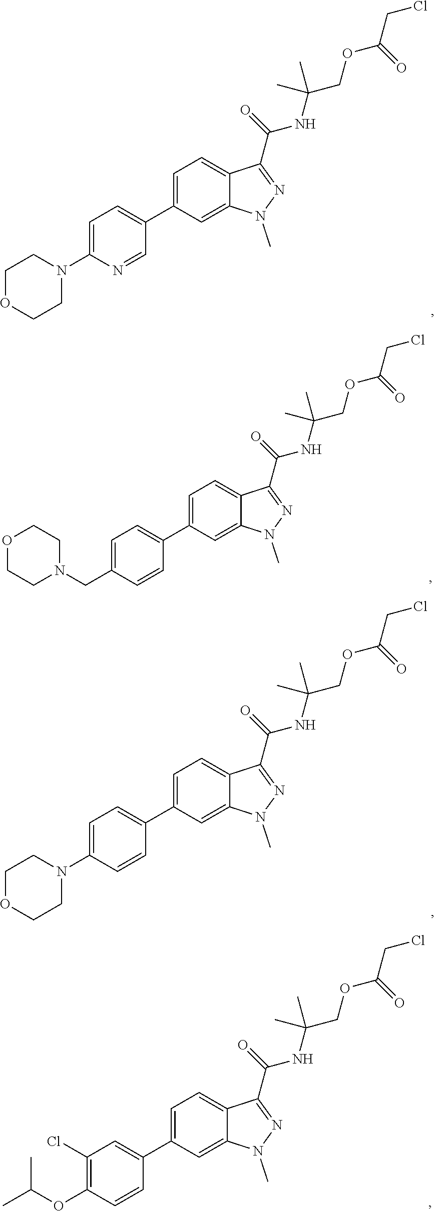 [SMILES: Cn1nc(C(=O)NC(C)(C)COC(=O)CCl)c2ccc(-c3ccc(N4CCOCC4)nc3)cc21 ; Cn1nc(C(=O)NC(C)(C)COC(=O)CCl)c2ccc(-c3ccc(CN4CCOCC4)cc3)cc21 ; Cn1nc(C(=O)NC(C)(C)COC(=O)CCl)c2ccc(-c3ccc(N4CCOCC4)cc3)cc21 ; CC(C)Oc1ccc(-c2ccc3c(C(=O)NC(C)(C)COC(=O)CCl)nn(C)c3c2)cc1Cl]

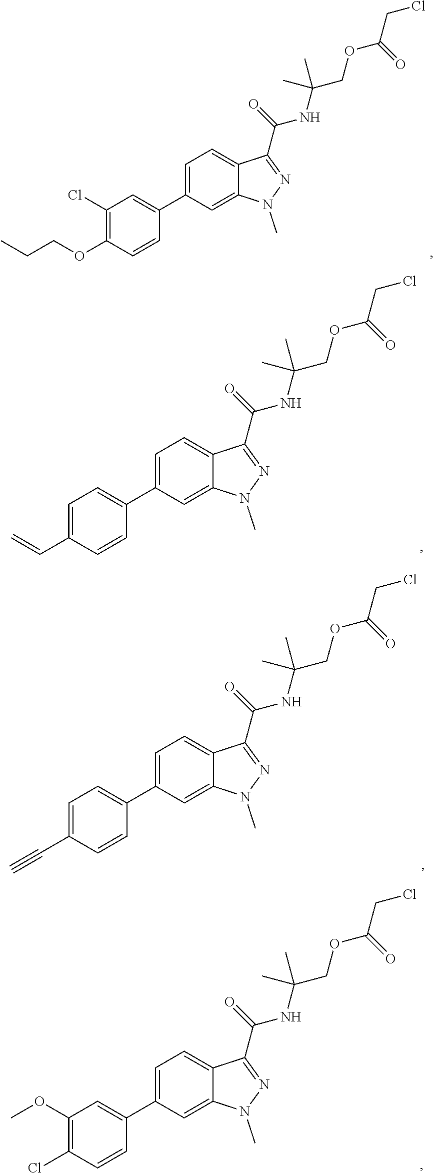 [SMILES: CCCOc1ccc(-c2ccc3c(C(=O)NC(C)(C)COC(=O)CCl)nn(C)c3c2)cc1Cl ; C=Cc1ccc(-c2ccc3c(C(=O)NC(C)(C)COC(=O)CCl)nn(C)c3c2)cc1 ; C#Cc1ccc(-c2ccc3c(C(=O)NC(C)(C)COC(=O)CCl)nn(C)c3c2)cc1 ; COc1cc(-c2ccc3c(C(=O)NC(C)(C)COC(=O)CCl)nn(C)c3c2)ccc1Cl]

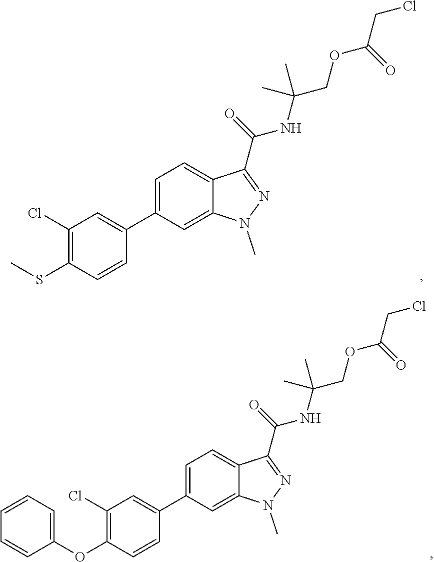 [SMILES: CSc1ccc(-c2ccc3c(C(=O)NC(C)(C)COC(=O)CCl)nn(C)c3c2)cc1Cl ; Cn1nc(C(=O)NC(C)(C)COC(=O)CCl)c2ccc(-c3ccc(Oc4ccccc4)c(Cl)c3)cc21]

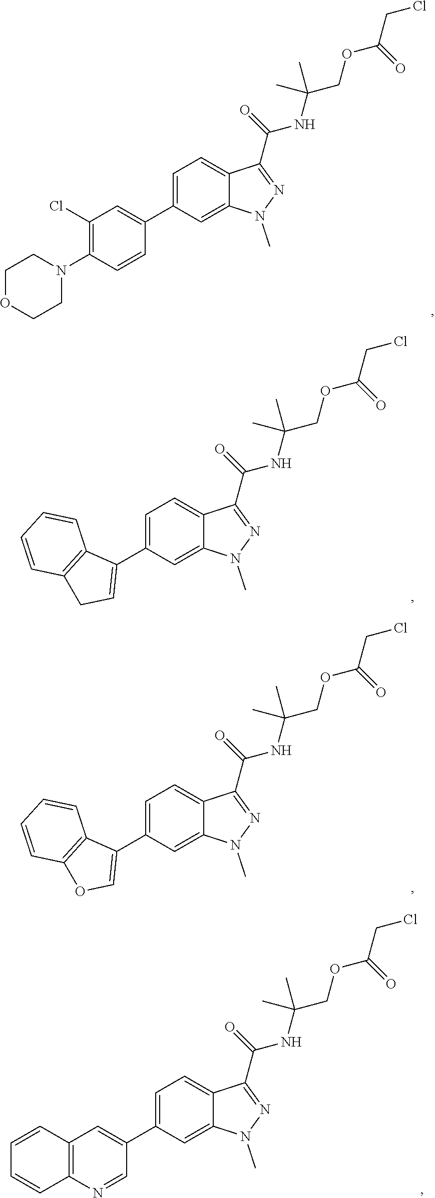 [SMILES: Cn1nc(C(=O)NC(C)(C)COC(=O)CCl)c2ccc(-c3ccc(N4CCOCC4)c(Cl)c3)cc21 ; Cn1nc(C(=O)NC(C)(C)COC(=O)CCl)c2ccc(C3=CCc4ccccc43)cc21 ; Cn1nc(C(=O)NC(C)(C)COC(=O)CCl)c2ccc(-c3coc4ccccc34)cc21 ; Cn1nc(C(=O)NC(C)(C)COC(=O)CCl)c2ccc(-c3cnc4ccccc4c3)cc21]

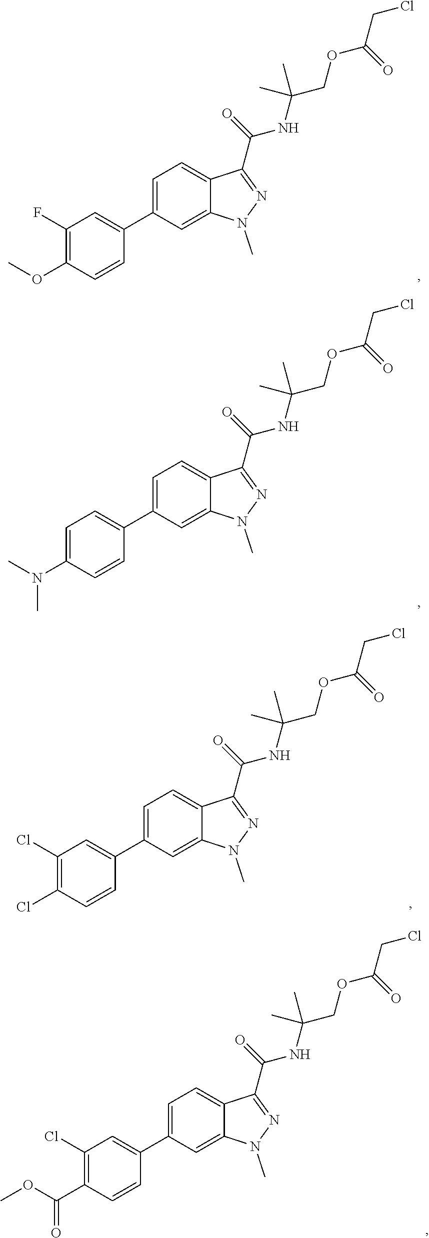 [SMILES: COc1ccc(-c2ccc3c(C(=O)NC(C)(C)COC(=O)CCl)nn(C)c3c2)cc1F ; CN(C)c1ccc(-c2ccc3c(C(=O)NC(C)(C)COC(=O)CCl)nn(C)c3c2)cc1 ; Cn1nc(C(=O)NC(C)(C)COC(=O)CCl)c2ccc(-c3ccc(Cl)c(Cl)c3)cc21 ; COC(=O)c1ccc(-c2ccc3c(C(=O)NC(C)(C)COC(=O)CCl)nn(C)c3c2)cc1Cl]

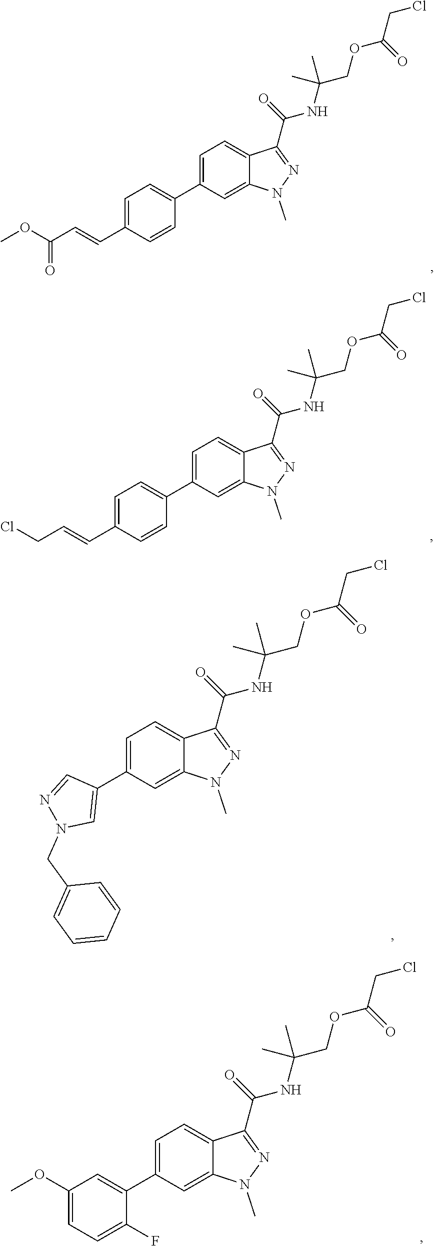 [SMILES: COC(=O)/C=C/c1ccc(-c2ccc3c(C(=O)NC(C)(C)COC(=O)CCl)nn(C)c3c2)cc1 ; Cn1nc(C(=O)NC(C)(C)COC(=O)CCl)c2ccc(-c3ccc(/C=C/CCl)cc3)cc21 ; Cn1nc(C(=O)NC(C)(C)COC(=O)CCl)c2ccc(-c3cnn(Cc4ccccc4)c3)cc21 ; COc1ccc(F)c(-c2ccc3c(C(=O)NC(C)(C)COC(=O)CCl)nn(C)c3c2)c1]

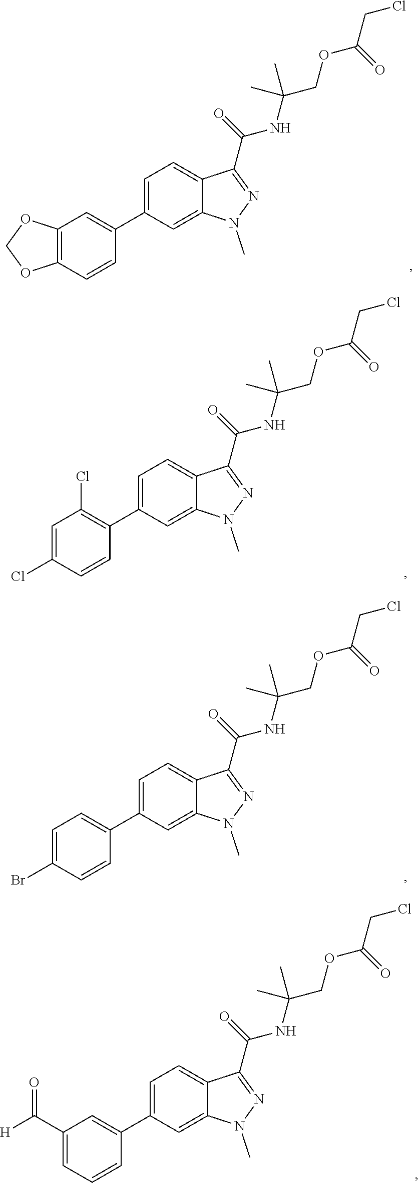 [SMILES: Cn1nc(C(=O)NC(C)(C)COC(=O)CCl)c2ccc(-c3ccc4c(c3)OCO4)cc21 ; Cn1nc(C(=O)NC(C)(C)COC(=O)CCl)c2ccc(-c3ccc(Cl)cc3Cl)cc21 ; Cn1nc(C(=O)NC(C)(C)COC(=O)CCl)c2ccc(-c3ccc(Br)cc3)cc21 ; Cn1nc(C(=O)NC(C)(C)COC(=O)CCl)c2ccc(-c3cccc(C=O)c3)cc21]

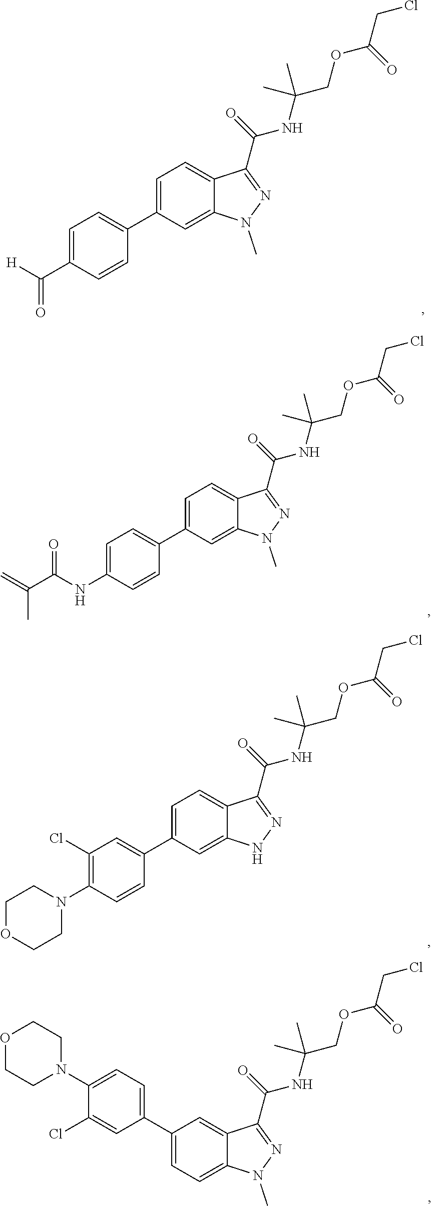 [SMILES: Cn1nc(C(=O)NC(C)(C)COC(=O)CCl)c2ccc(-c3ccc(C=O)cc3)cc21 ; C=C(C)C(=O)Nc1ccc(-c2ccc3c(C(=O)NC(C)(C)COC(=O)CCl)nn(C)c3c2)cc1 ; CC(C)(COC(=O)CCl)NC(=O)c1n[nH]c2cc(-c3ccc(N4CCOCC4)c(Cl)c3)ccc12 ; Cn1nc(C(=O)NC(C)(C)COC(=O)CCl)c2cc(-c3ccc(N4CCOCC4)c(Cl)c3)ccc21]

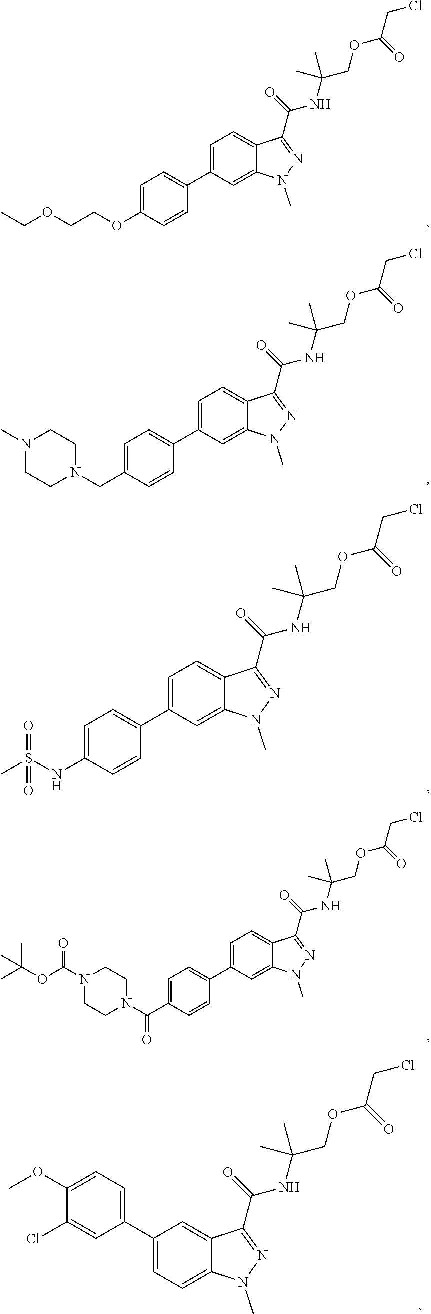 [SMILES: CCOCCOc1ccc(-c2ccc3c(C(=O)NC(C)(C)COC(=O)CCl)nn(C)c3c2)cc1 ; CN1CCN(Cc2ccc(-c3ccc4c(C(=O)NC(C)(C)COC(=O)CCl)nn(C)c4c3)cc2)CC1 ; Cn1nc(C(=O)NC(C)(C)COC(=O)CCl)c2ccc(-c3ccc(NS(C)(=O)=O)cc3)cc21 ; Cn1nc(C(=O)NC(C)(C)COC(=O)CCl)c2ccc(-c3ccc(C(=O)N4CCN(C(=O)OC(C)(C)C)CC4)cc3)cc21 ; COc1ccc(-c2ccc3c(c2)c(C(=O)NC(C)(C)COC(=O)CCl)nn3C)cc1Cl]

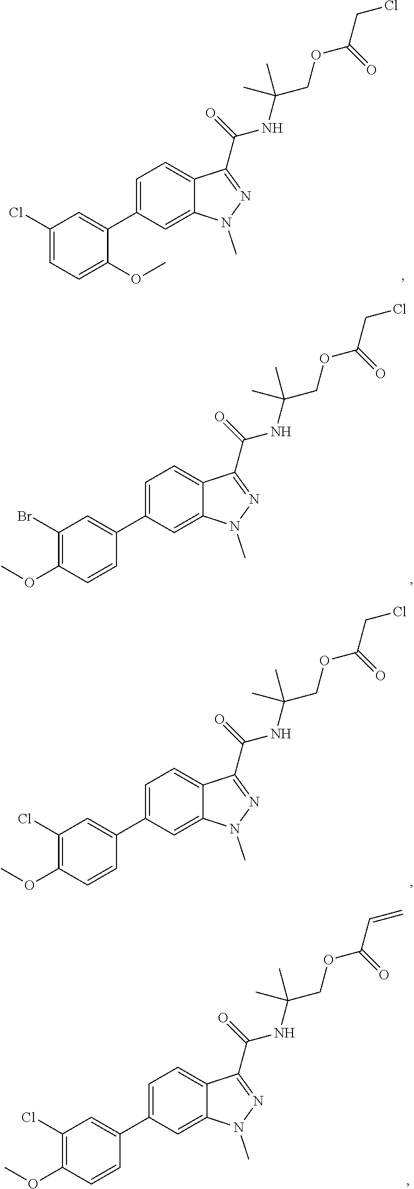 [SMILES: COc1ccc(Cl)cc1-c1ccc2c(C(=O)NC(C)(C)COC(=O)CCl)nn(C)c2c1 ; COc1ccc(-c2ccc3c(C(=O)NC(C)(C)COC(=O)CCl)nn(C)c3c2)cc1Br ; COc1ccc(-c2ccc3c(C(=O)NC(C)(C)COC(=O)CCl)nn(C)c3c2)cc1Cl ; C=CC(=O)OCC(C)(C)NC(=O)c1nn(C)c2cc(-c3ccc(OC)c(Cl)c3)ccc12]

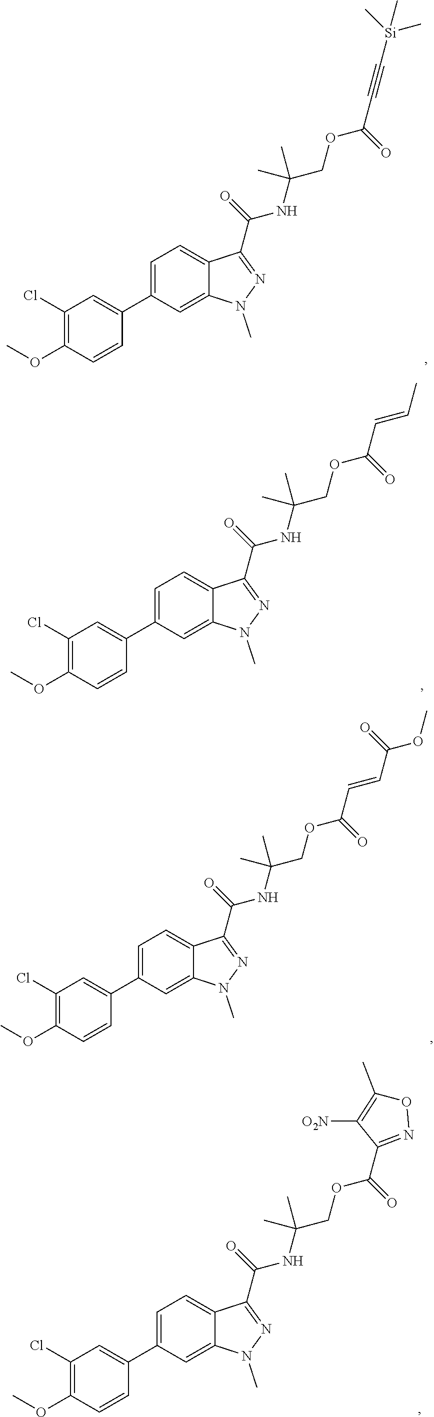

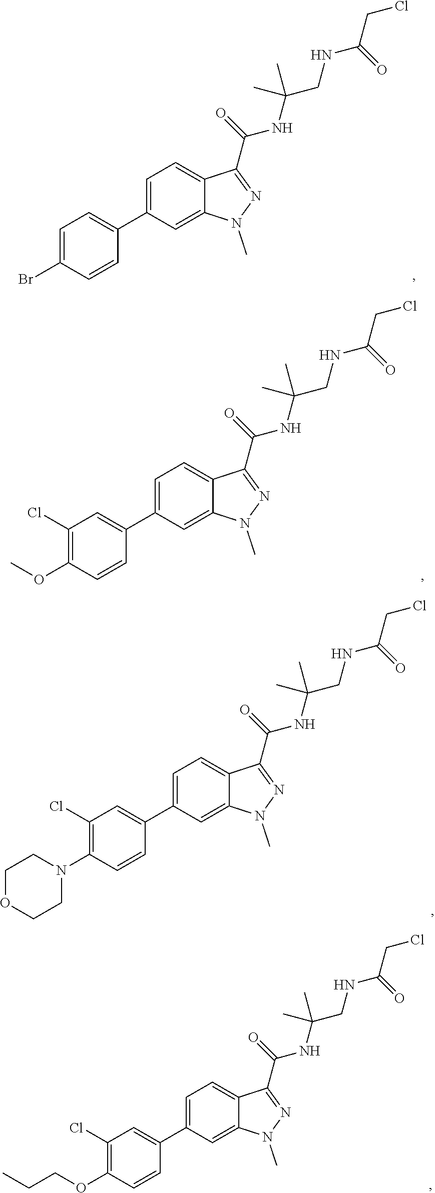 [SMILES: Cn1nc(C(=O)NC(C)(C)CNC(=O)CCl)c2ccc(-c3ccc(Br)cc3)cc21 ; COc1ccc(-c2ccc3c(C(=O)NC(C)(C)CNC(=O)CCl)nn(C)c3c2)cc1Cl ; Cn1nc(C(=O)NC(C)(C)CNC(=O)CCl)c2ccc(-c3ccc(N4CCOCC4)c(Cl)c3)cc21 ; CCCOc1ccc(-c2ccc3c(C(=O)NC(C)(C)CNC(=O)CCl)nn(C)c3c2)cc1Cl]

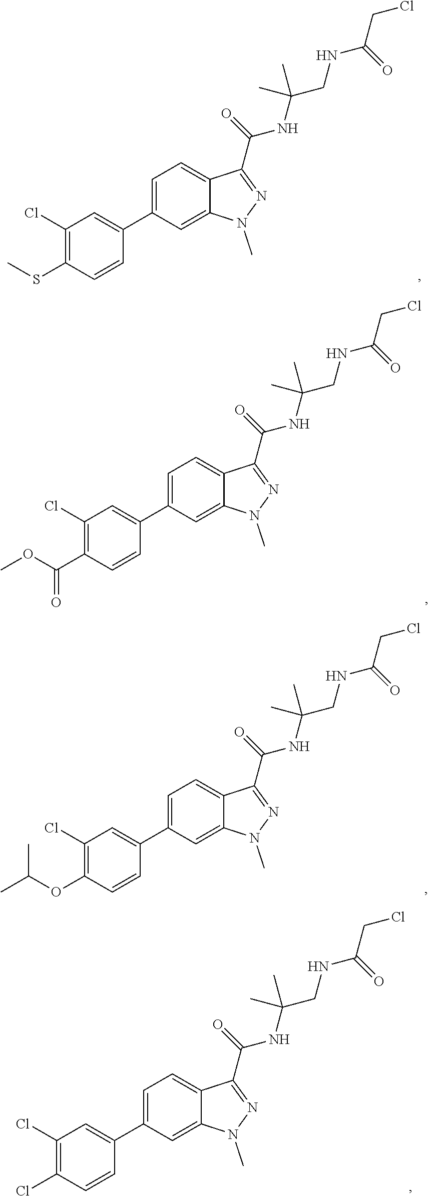 [SMILES: CSc1ccc(-c2ccc3c(C(=O)NC(C)(C)CNC(=O)CCl)nn(C)c3c2)cc1Cl ; COC(=O)c1ccc(-c2ccc3c(C(=O)NC(C)(C)CNC(=O)CCl)nn(C)c3c2)cc1Cl ; CC(C)Oc1ccc(-c2ccc3c(C(=O)NC(C)(C)CNC(=O)CCl)nn(C)c3c2)cc1Cl ; Cn1nc(C(=O)NC(C)(C)CNC(=O)CCl)c2ccc(-c3ccc(Cl)c(Cl)c3)cc21]

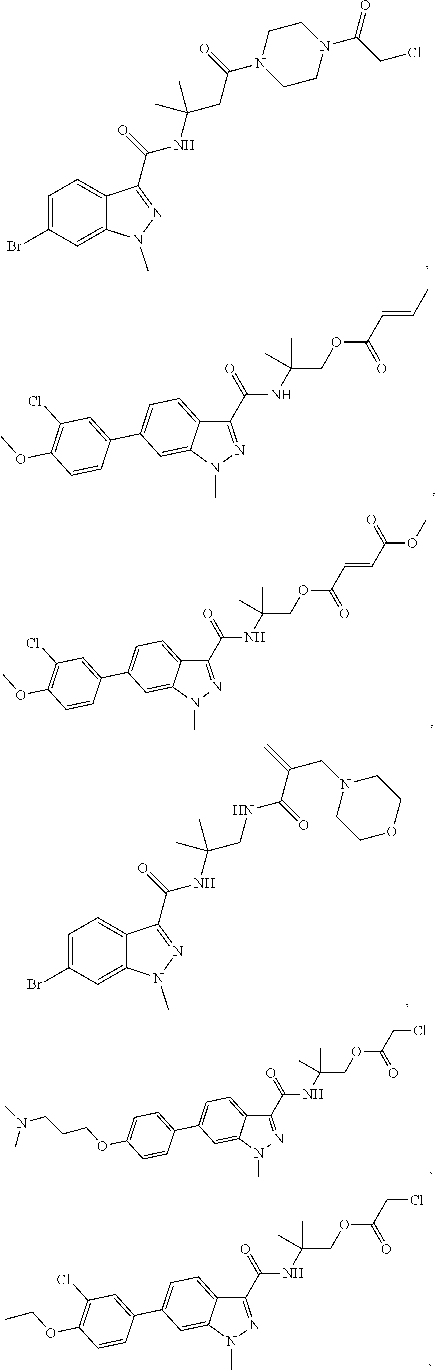

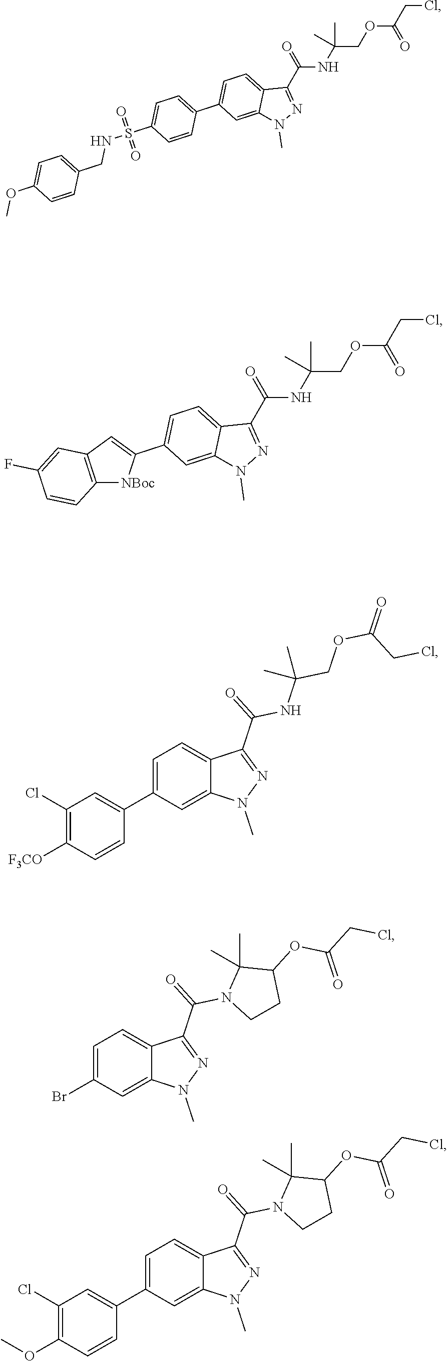 [SMILES: COc1ccc(CNS(=O)(=O)c2ccc(-c3ccc4c(C(=O)NC(C)(C)COC(=O)CCl)nn(C)c4c3)cc2)cc1 ; Cn1nc(C(=O)NC(C)(C)COC(=O)CCl)c2ccc(-c3cc4cc(F)ccc4n3C(=O)OC(C)(C)C)cc21 ; Cn1nc(C(=O)NC(C)(C)COC(=O)CCl)c2ccc(-c3ccc(OC(F)(F)F)c(Cl)c3)cc21 ; Cn1nc(C(=O)N2CCC(OC(=O)CCl)C2(C)C)c2ccc(Br)cc21 ; COc1ccc(-c2ccc3c(C(=O)N4CCC(OC(=O)CCl)C4(C)C)nn(C)c3c2)cc1Cl]

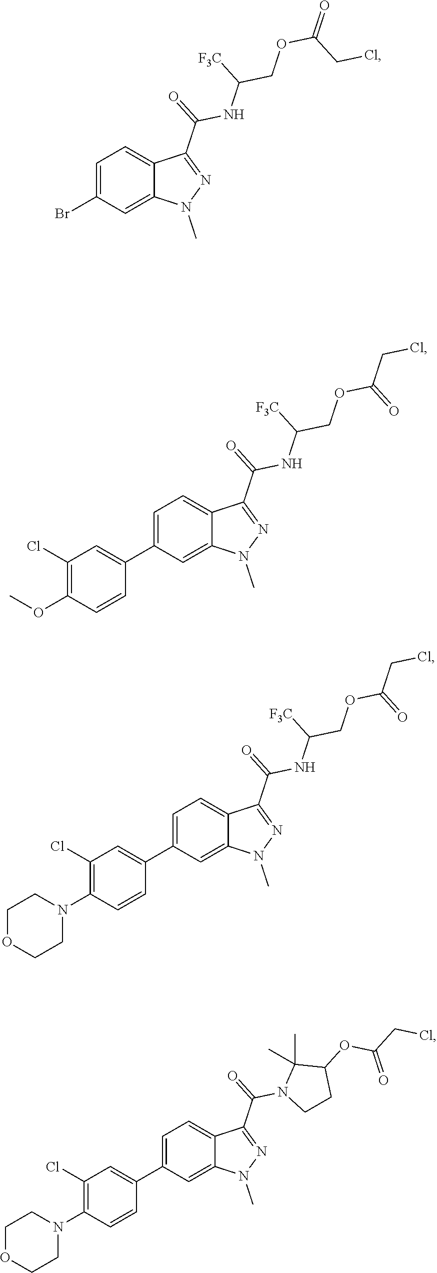 [SMILES: Cn1nc(C(=O)NC(COC(=O)CCl)C(F)(F)F)c2ccc(Br)cc21 ; COc1ccc(-c2ccc3c(C(=O)NC(COC(=O)CCl)C(F)(F)F)nn(C)c3c2)cc1Cl ; Cn1nc(C(=O)NC(COC(=O)CCl)C(F)(F)F)c2ccc(-c3ccc(N4CCOCC4)c(Cl)c3)cc21 ; Cn1nc(C(=O)N2CCC(OC(=O)CCl)C2(C)C)c2ccc(-c3ccc(N4CCOCC4)c(Cl)c3)cc21]

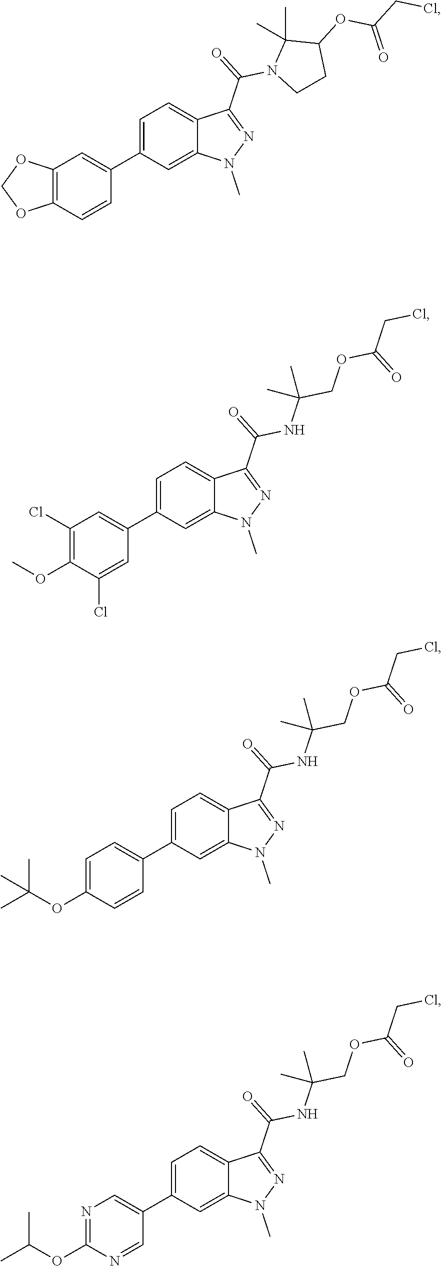 [SMILES: Cn1nc(C(=O)N2CCC(OC(=O)CCl)C2(C)C)c2ccc(-c3ccc4c(c3)OCO4)cc21 ; COc1c(Cl)cc(-c2ccc3c(C(=O)NC(C)(C)COC(=O)CCl)nn(C)c3c2)cc1Cl ; Cn1nc(C(=O)NC(C)(C)COC(=O)CCl)c2ccc(-c3ccc(OC(C)(C)C)cc3)cc21 ; CC(C)Oc1ncc(-c2ccc3c(C(=O)NC(C)(C)COC(=O)CCl)nn(C)c3c2)cn1]

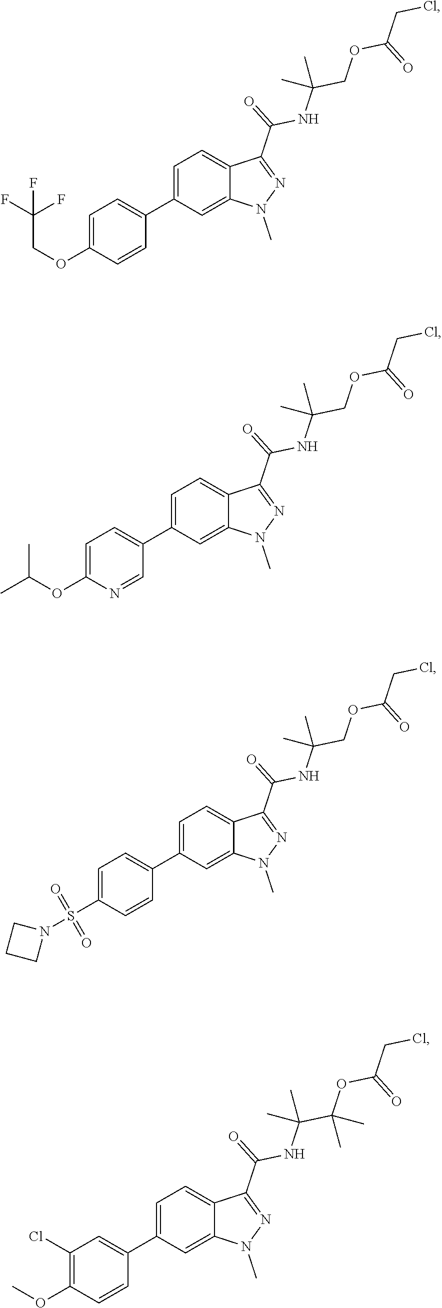 [SMILES: Cn1nc(C(=O)NC(C)(C)COC(=O)CCl)c2ccc(-c3ccc(OCC(F)(F)F)cc3)cc21 ; CC(C)Oc1ccc(-c2ccc3c(C(=O)NC(C)(C)COC(=O)CCl)nn(C)c3c2)cn1 ; Cn1nc(C(=O)NC(C)(C)COC(=O)CCl)c2ccc(-c3ccc(S(=O)(=O)N4CCC4)cc3)cc21 ; COc1ccc(-c2ccc3c(C(=O)NC(C)(C)C(C)(C)OC(=O)CCl)nn(C)c3c2)cc1Cl]

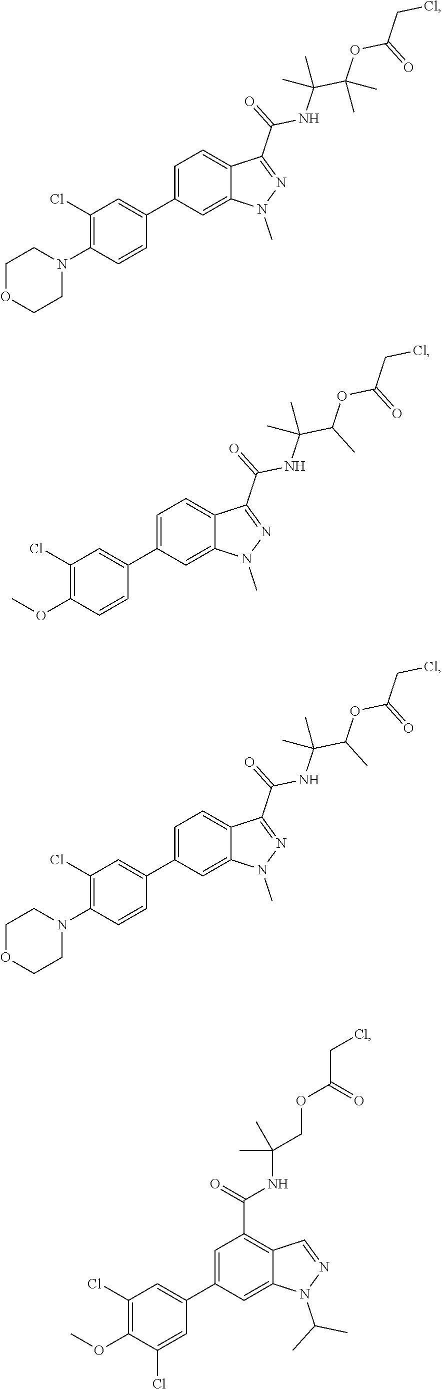 [SMILES: Cn1nc(C(=O)NC(C)(C)C(C)(C)OC(=O)CCl)c2ccc(-c3ccc(N4CCOCC4)c(Cl)c3)cc21 ; COc1ccc(-c2ccc3c(C(=O)NC(C)(C)C(C)OC(=O)CCl)nn(C)c3c2)cc1Cl ; CC(OC(=O)CCl)C(C)(C)NC(=O)c1nn(C)c2cc(-c3ccc(N4CCOCC4)c(Cl)c3)ccc12 ; COc1c(Cl)cc(-c2cc(C(=O)NC(C)(C)COC(=O)CCl)c3cnn(C(C)C)c3c2)cc1Cl]

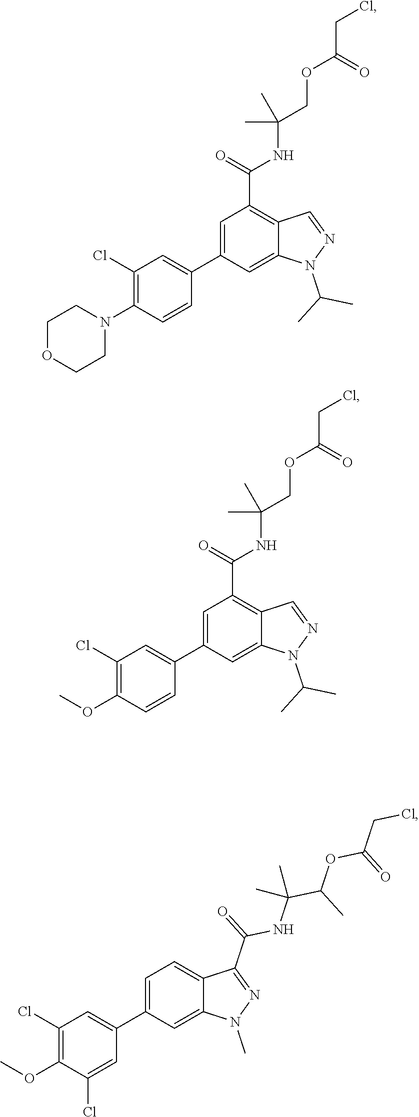 [SMILES: CC(C)n1ncc2c(C(=O)NC(C)(C)COC(=O)CCl)cc(-c3ccc(N4CCOCC4)c(Cl)c3)cc21 ; COc1ccc(-c2cc(C(=O)NC(C)(C)COC(=O)CCl)c3cnn(C(C)C)c3c2)cc1Cl ; COc1c(Cl)cc(-c2ccc3c(C(=O)NC(C)(C)C(C)OC(=O)CCl)nn(C)c3c2)cc1Cl]

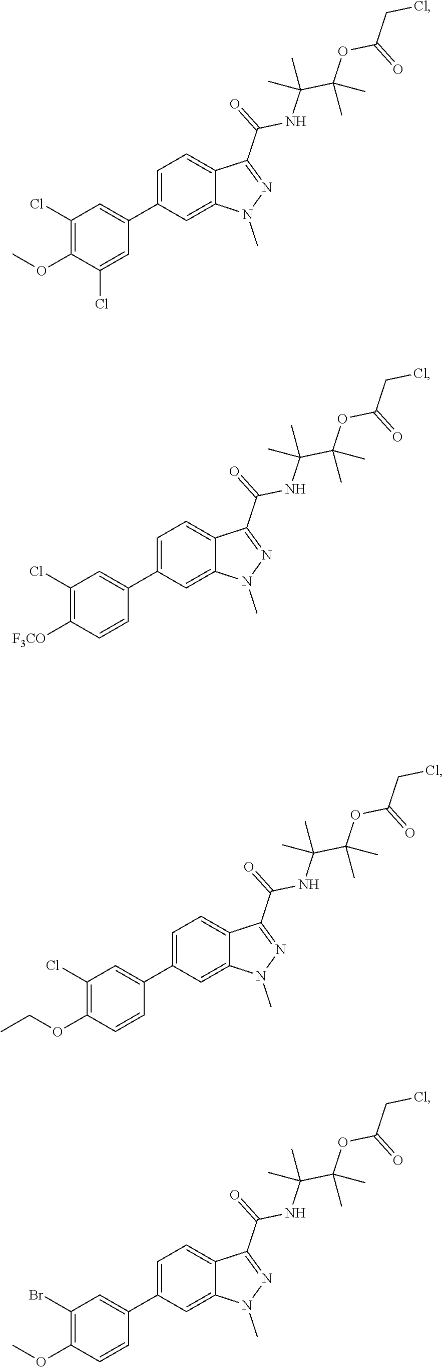 [SMILES: COc1c(Cl)cc(-c2ccc3c(C(=O)NC(C)(C)C(C)(C)OC(=O)CCl)nn(C)c3c2)cc1Cl ; Cn1nc(C(=O)NC(C)(C)C(C)(C)OC(=O)CCl)c2ccc(-c3ccc(OC(F)(F)F)c(Cl)c3)cc21 ; CCOc1ccc(-c2ccc3c(C(=O)NC(C)(C)C(C)(C)OC(=O)CCl)nn(C)c3c2)cc1Cl ; COc1ccc(-c2ccc3c(C(=O)NC(C)(C)C(C)(C)OC(=O)CCl)nn(C)c3c2)cc1Br]

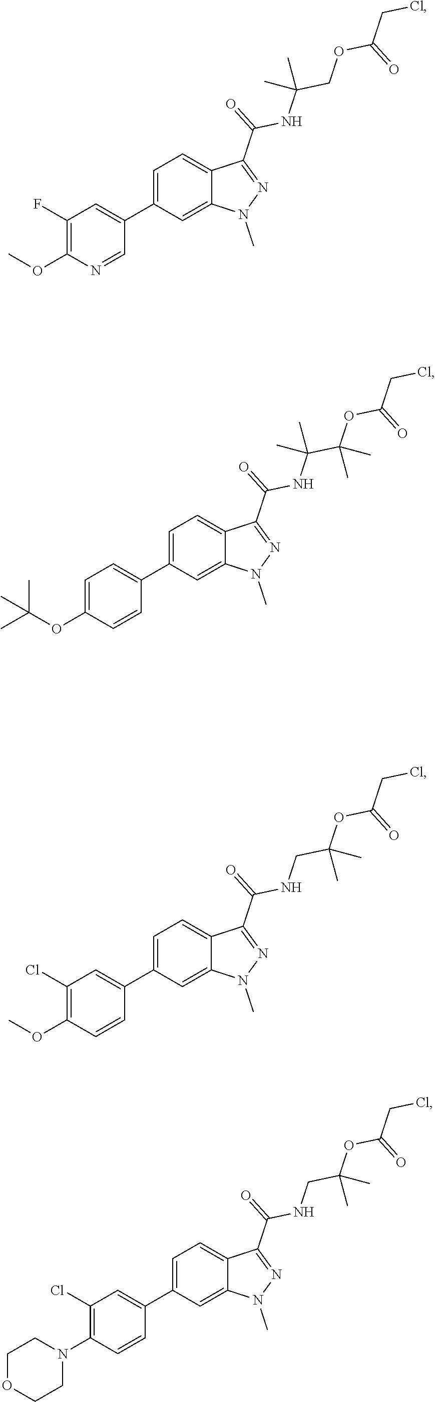 [SMILES: COc1ncc(-c2ccc3c(C(=O)NC(C)(C)COC(=O)CCl)nn(C)c3c2)cc1F ; Cn1nc(C(=O)NC(C)(C)C(C)(C)OC(=O)CCl)c2ccc(-c3ccc(OC(C)(C)C)cc3)cc21 ; COc1ccc(-c2ccc3c(C(=O)NCC(C)(C)OC(=O)CCl)nn(C)c3c2)cc1Cl ; Cn1nc(C(=O)NCC(C)(C)OC(=O)CCl)c2ccc(-c3ccc(N4CCOCC4)c(Cl)c3)cc21]

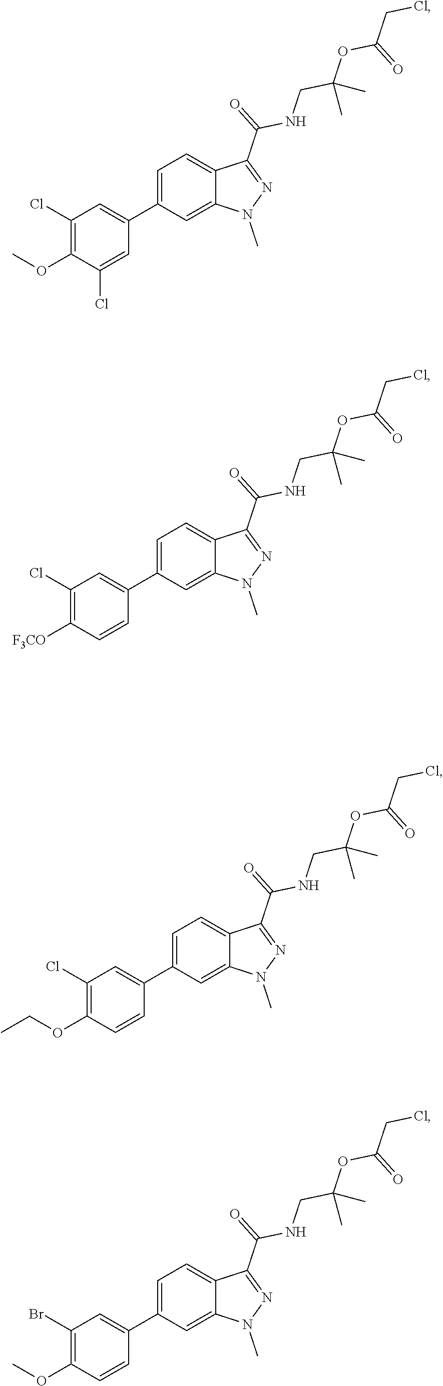 [SMILES: COc1c(Cl)cc(-c2ccc3c(C(=O)NCC(C)(C)OC(=O)CCl)nn(C)c3c2)cc1Cl ; Cn1nc(C(=O)NCC(C)(C)OC(=O)CCl)c2ccc(-c3ccc(OC(F)(F)F)c(Cl)c3)cc21 ; CCOc1ccc(-c2ccc3c(C(=O)NCC(C)(C)OC(=O)CCl)nn(C)c3c2)cc1Cl ; COc1ccc(-c2ccc3c(C(=O)NCC(C)(C)OC(=O)CCl)nn(C)c3c2)cc1Br]

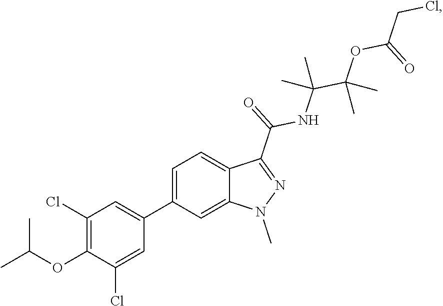 [SMILES: CC(C)Oc1c(Cl)cc(-c2ccc3c(C(=O)NC(C)(C)C(C)(C)OC(=O)CCl)nn(C)c3c2)cc1Cl]

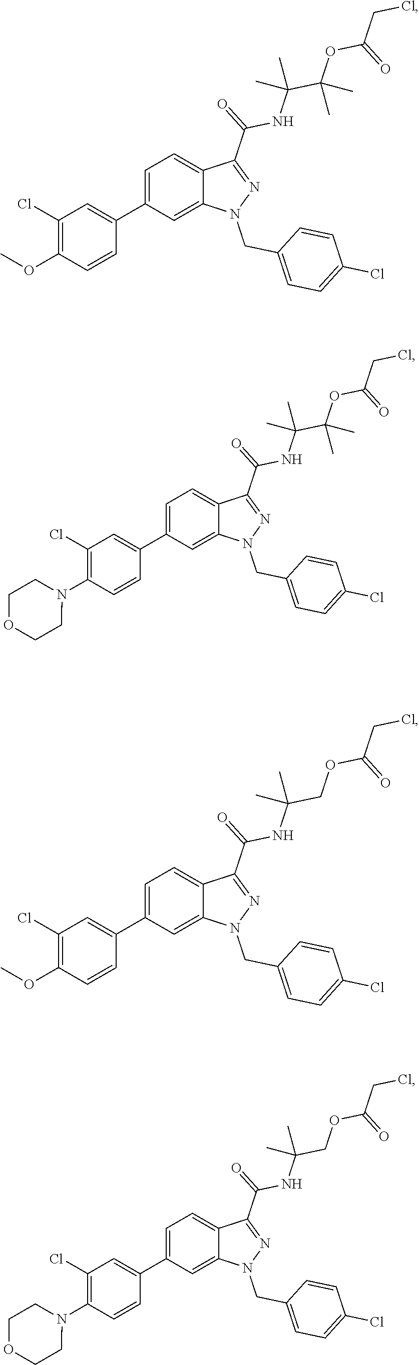 [SMILES: COc1ccc(-c2ccc3c(C(=O)NC(C)(C)C(C)(C)OC(=O)CCl)nn(Cc4ccc(Cl)cc4)c3c2)cc1Cl ; CC(C)(NC(=O)c1nn(Cc2ccc(Cl)cc2)c2cc(-c3ccc(N4CCOCC4)c(Cl)c3)ccc12)C(C)(C)OC(=O)CCl ; COc1ccc(-c2ccc3c(C(=O)NC(C)(C)COC(=O)CCl)nn(Cc4ccc(Cl)cc4)c3c2)cc1Cl ; CC(C)(COC(=O)CCl)NC(=O)c1nn(Cc2ccc(Cl)cc2)c2cc(-c3ccc(N4CCOCC4)c(Cl)c3)ccc12]

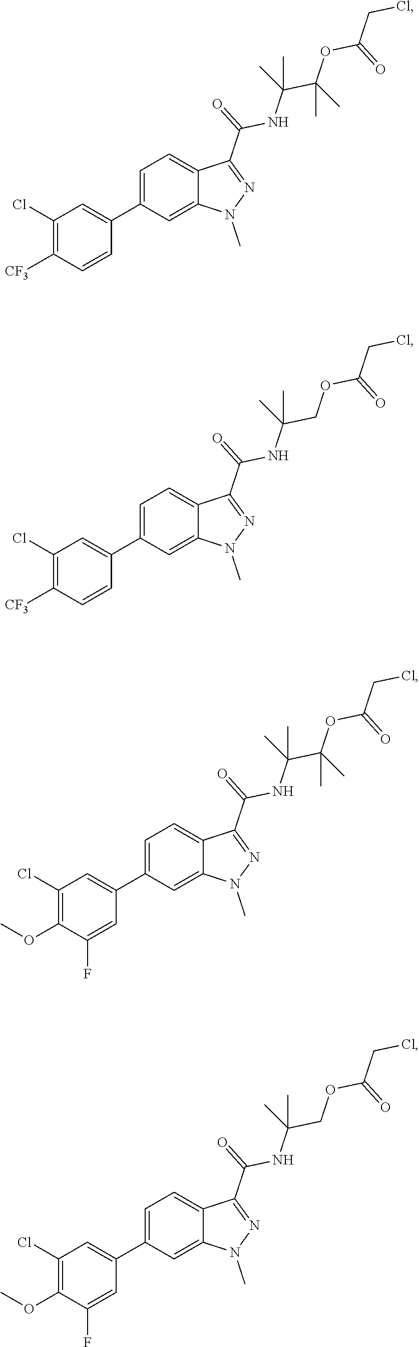 [SMILES: Cn1nc(C(=O)NC(C)(C)C(C)(C)OC(=O)CCl)c2ccc(-c3ccc(C(F)(F)F)c(Cl)c3)cc21 ; Cn1nc(C(=O)NC(C)(C)COC(=O)CCl)c2ccc(-c3ccc(C(F)(F)F)c(Cl)c3)cc21 ; COc1c(F)cc(-c2ccc3c(C(=O)NC(C)(C)C(C)(C)OC(=O)CCl)nn(C)c3c2)cc1Cl ; COc1c(F)cc(-c2ccc3c(C(=O)NC(C)(C)COC(=O)CCl)nn(C)c3c2)cc1Cl]

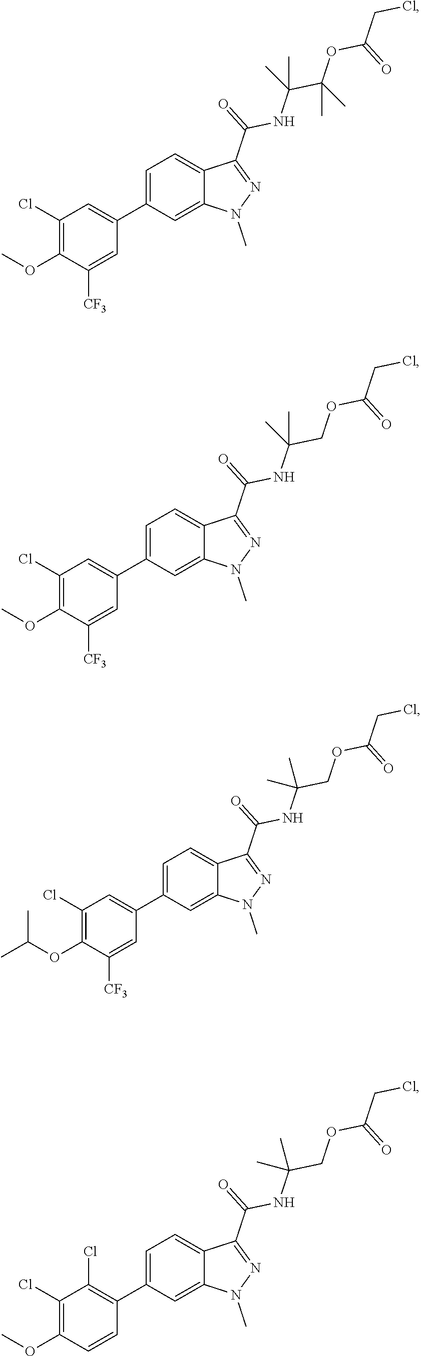 [SMILES: COc1c(Cl)cc(-c2ccc3c(C(=O)NC(C)(C)C(C)(C)OC(=O)CCl)nn(C)c3c2)cc1C(F)(F)F ; COc1c(Cl)cc(-c2ccc3c(C(=O)NC(C)(C)COC(=O)CCl)nn(C)c3c2)cc1C(F)(F)F ; CC(C)Oc1c(Cl)cc(-c2ccc3c(C(=O)NC(C)(C)COC(=O)CCl)nn(C)c3c2)cc1C(F)(F)F ; COc1ccc(-c2ccc3c(C(=O)NC(C)(C)COC(=O)CCl)nn(C)c3c2)c(Cl)c1Cl]

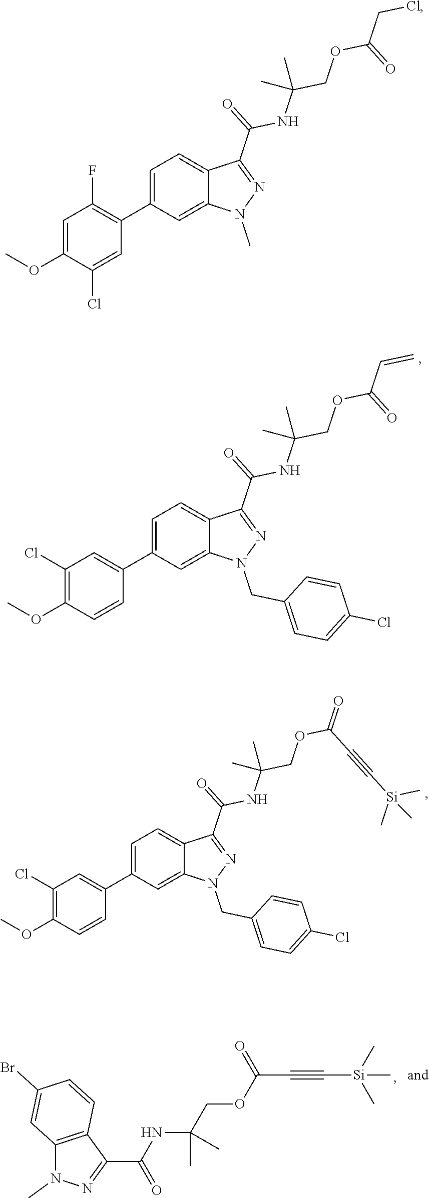

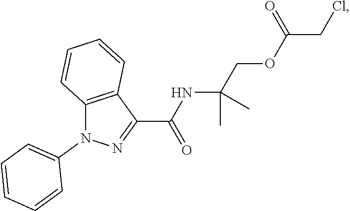 [SMILES: CC(C)(COC(=O)CCl)NC(=O)c1nn(-c2ccccc2)c2ccccc12]

or an N-oxide, crystalline form, hydrate, or pharmaceutically acceptable salt thereof.

In some embodiments, the compound has a structure selected from the group consisting of:

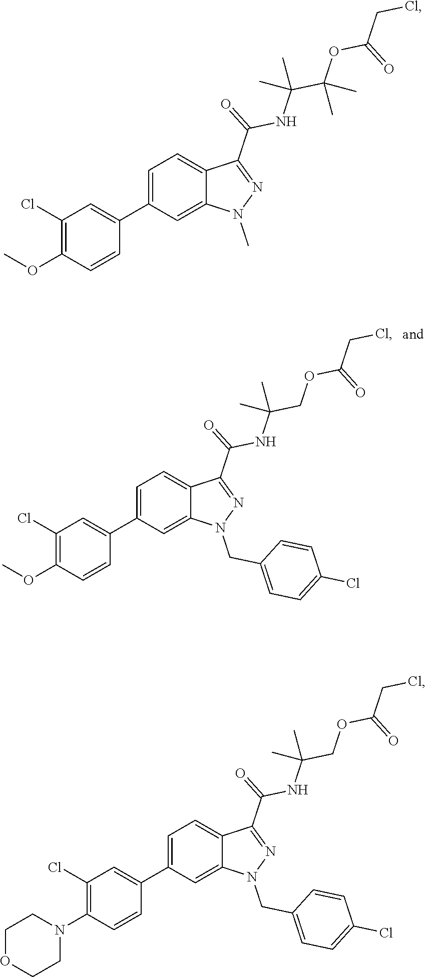 [SMILES: COc1ccc(-c2ccc3c(C(=O)NC(C)(C)C(C)(C)OC(=O)CCl)nn(C)c3c2)cc1Cl ; COc1ccc(-c2ccc3c(C(=O)NC(C)(C)COC(=O)CCl)nn(Cc4ccc(Cl)cc4)c3c2)cc1Cl ; CC(C)(COC(=O)CCl)NC(=O)c1nn(Cc2ccc(Cl)cc2)c2cc(-c3ccc(N4CCOCC4)c(Cl)c3)ccc12]

or an N-oxide, crystalline form, hydrate, or pharmaceutically acceptable salt thereof.

Another embodiment of the present disclosure is a compound according to formula (2):

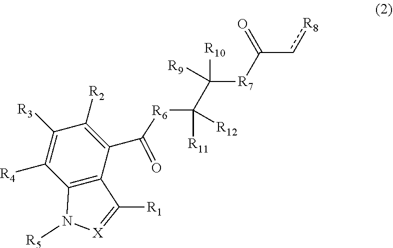

wherein:
a dashed line indicates the presence of an optional double bond;
X is selected from the group consisting of O, N, S, and C;
R₁, R₂, R₃, R₄ and R₅ are independently selected from the group consisting of H, D, —OH, halo, ether, ester, furanyl, indole, indazole, pyrrole, pyrazole, pyridine, pyrimidine, naphthalene, indene, dibenzofuran, benzodioxole, amide, —(O)C(R), —C(O)OR, —NO₂, alkyl, aryl, alkyl-aryl, alkyl-heteroaryl, alkenyl, alkenyl-aryl, alkenyl-heteroaryl, peptide, and polypeptide, wherein the ether, ester, furanyl, indole, indazole, pyrrole, pyrazole, pyridine, pyrimidine, naphthalene, indene, dibenzofuran, benzodioxole, amide, alkyl, aryl, alkyl-aryl, alkyl-heteroaryl, alkenyl, alkenyl-aryl, alkenyl-heteroaryl, peptide, and polypeptide may be optionally substituted with an atom or a group selected from the group consisting of O, N, S, epoxy, —OH, halo, —CN, —NO₂, C₁₋₄alkyl, CF₃, —CF₃O, carbonyl, alkyl-aryl, thiol, methylmethanesulfonamide, methylthiol, dioxane, tetrahydropyzan, morpholinyl, piperazine, sulfonyl, oxazole, oxadiazole, dizaole, triazole, amide, ether, ester and combinations thereof; or R₁ and R₂ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₂ and R₃ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₃ and R₄ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof,
R₆ and R₇ are independently selected from the group consisting of C, N, O and S; or R₆ and R₇ may together form a saturated or unsaturated ring structure that may be optionally substituted with an atom or a group from the group consisting of O, N, halo, C₁₋₄alkyl, CF₃, oxazole, oxadiazole, dizaole, triazole, amide, ether, ester and combinations thereof,
R₈ is selected from the group consisting of H, D, C, O, N, alkenyl, alkynyl, ester, oxazole, piperazine, morpholinyl and halo,
R₉, R₁₀, R₁₁ and R₁₂ are independently selected from the group consisting of no atom, H, D, —OH, halo, —CN, —NO₂, C₁₋₄alkyl, CF₃, —CF₃O, carbonyl, aryl, alkyl-aryl, thiol, ether, and ester,
wherein R is selected from the group consisting of H, D, O, N, halo, oxazole, oxadiazole, dizaole, triazole, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of O, N, S, halo, C₁₋₄alkyl, CF₃, —CN and combinations thereof,
or an N-oxide, crystalline form, hydrate, or pharmaceutically acceptable salt thereof.

Another embodiment of the present disclosure is a compound according to formula (3):

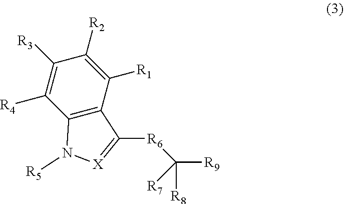

wherein:
X is selected from the group consisting of O, N, S, and C;
R₁, R₂, R₃, R₄, R₅ and R₉ are independently selected from the group consisting of H, D, —OH, halo, ether, ester, furanyl, indole, indazole, pyrrole, pyrazole, pyridine, pyrimidine, naphthalene, indene, dibenzofuran, benzodioxole, amide, —(O)C(R), —C(O)OR, —NO₂, alkyl, aryl, alkyl-aryl, alkyl-heteroaryl, alkenyl, alkenyl-aryl, alkenyl-heteroaryl, peptide, and polypeptide, wherein the ether, ester, furanyl, indole, indazole, pyrrole, pyrazole, pyridine, pyrimidine, naphthalene, indene, dibenzofuran, benzodioxole, amide, alkyl, aryl, alkyl-aryl, alkyl-heteroaryl, alkenyl, alkenyl-aryl, alkenyl-heteroaryl, peptide, and polypeptide may be optionally substituted with an atom or a group selected from the group consisting of O, N, S, epoxy, —OH, halo, —CN, —NO₂, C₁₋₄alkyl, CF₃, —CF₃O, carbonyl, alkyl-aryl, thiol, methylmethanesulfonamide, methylthiol, dioxane, tetrahydropyzan, morpholinyl, piperazine, sulfonyl, oxazole, oxadiazole, dizaole, triazole, amide, ether, ester and combinations thereof; or R₁ and R₂ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₂ and R₃ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₃ and R₄ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof,
R₆ is selected from the group consisting of amide, oxazole, oxadiazole, diazole, and triazole,
R₇ and R₈ are independently selected from the group consisting of no atom, H, D, —OH, halo, —CN, —NO₂, C₁₋₄alkyl, CF₃, —CF₃O, carbonyl, aryl, alkyl-aryl, thiol, ether, and ester,
wherein R is selected from the group consisting of H, D, O, N, halo, oxazole, oxadiazole, dizaole, triazole, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of O, N, S, halo, C₁₋₄alkyl, CF₃, —CN and combinations thereof,
or an N-oxide, crystalline form, hydrate, or pharmaceutically acceptable salt thereof.

In some embodiments, the compound has a structure selected from the group consisting of:

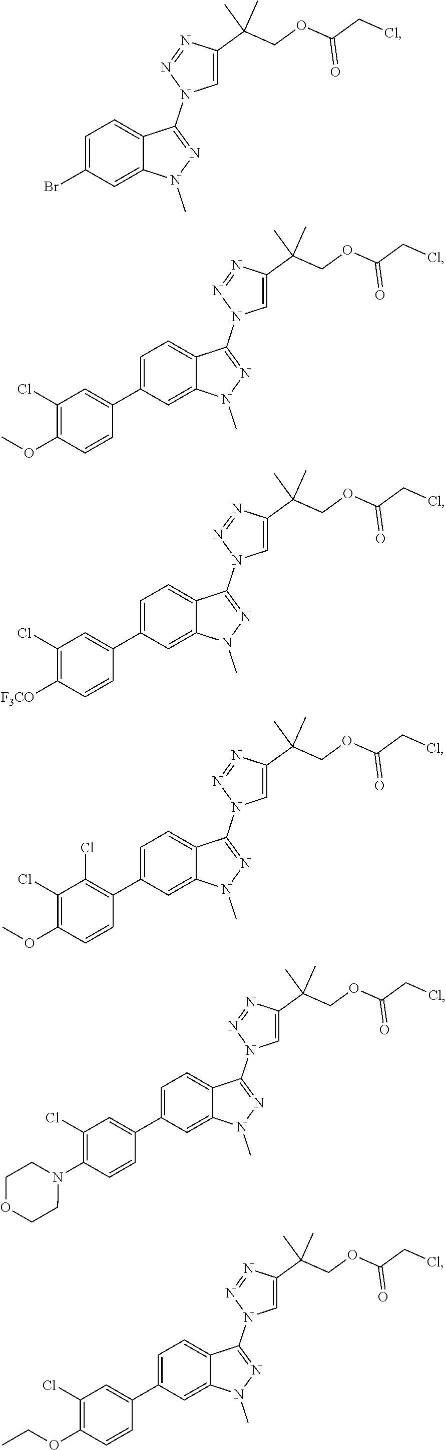 [SMILES: Cn1nc(-n2cc(C(C)(C)COC(=O)CCl)nn2)c2ccc(Br)cc21 ; COc1ccc(-c2ccc3c(-n4cc(C(C)(C)COC(=O)CCl)nn4)nn(C)c3c2)cc1Cl ; Cn1nc(-n2cc(C(C)(C)COC(=O)CCl)nn2)c2ccc(-c3ccc(OC(F)(F)F)c(Cl)c3)cc21 ; COc1ccc(-c2ccc3c(-n4cc(C(C)(C)COC(=O)CCl)nn4)nn(C)c3c2)c(Cl)c1Cl ; Cn1nc(-n2cc(C(C)(C)COC(=O)CCl)nn2)c2ccc(-c3ccc(N4CCOCC4)c(Cl)c3)cc21 ; CCOc1ccc(-c2ccc3c(-n4cc(C(C)(C)COC(=O)CCl)nn4)nn(C)c3c2)cc1Cl]

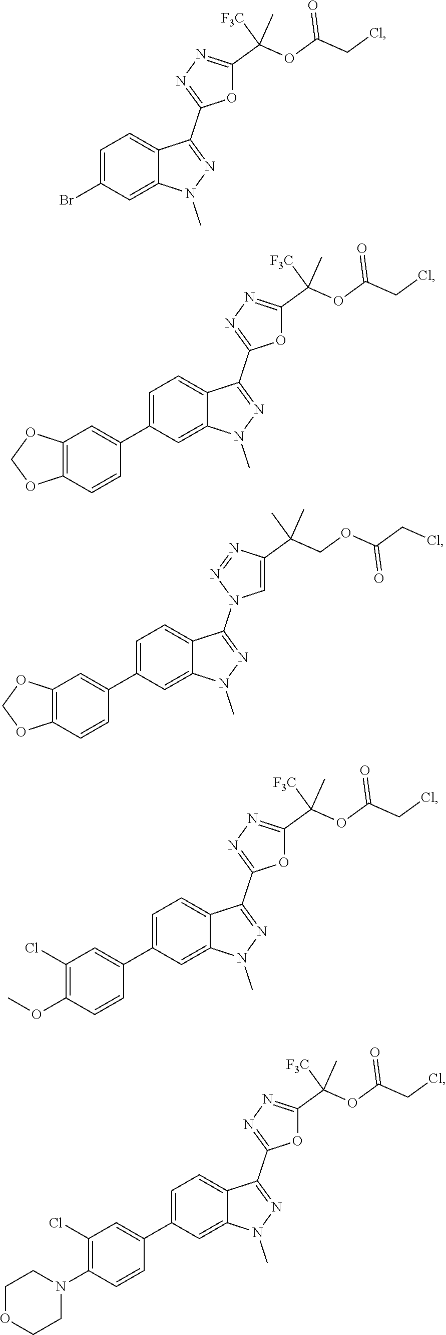 [SMILES: Cn1nc(-c2nnc(C(C)(OC(=O)CCl)C(F)(F)F)o2)c2ccc(Br)cc21 ; Cn1nc(-c2nnc(C(C)(OC(=O)CCl)C(F)(F)F)o2)c2ccc(-c3ccc4c(c3)OCO4)cc21 ; Cn1nc(-n2cc(C(C)(C)COC(=O)CCl)nn2)c2ccc(-c3ccc4c(c3)OCO4)cc21 ; COc1ccc(-c2ccc3c(-c4nnc(C(C)(OC(=O)CCl)C(F)(F)F)o4)nn(C)c3c2)cc1Cl ; Cn1nc(-c2nnc(C(C)(OC(=O)CCl)C(F)(F)F)o2)c2ccc(-c3ccc(N4CCOCC4)c(Cl)c3)cc21]

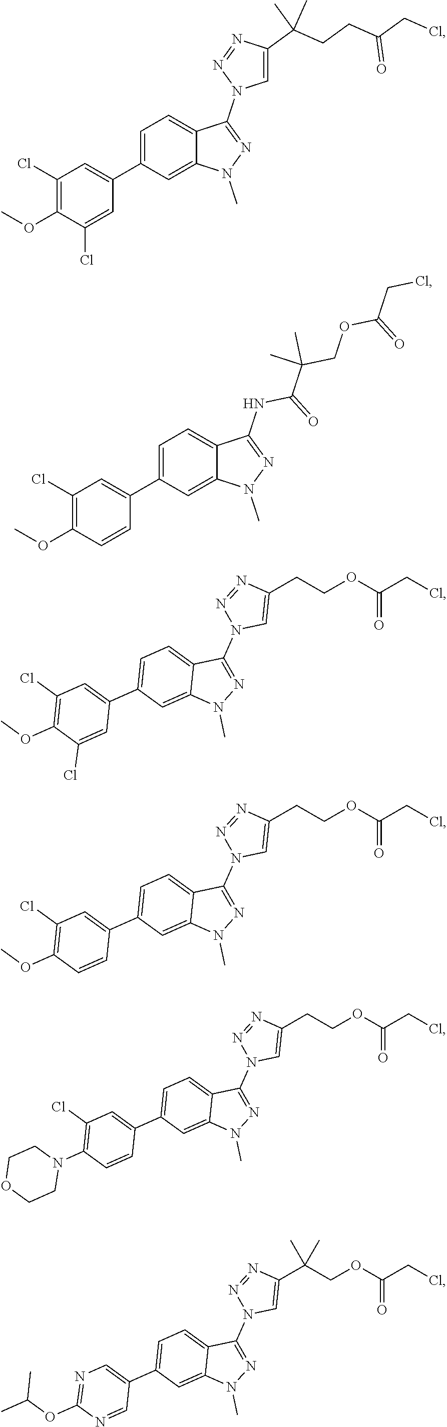 [SMILES: COc1c(Cl)cc(-c2ccc3c(-n4cc(C(C)(C)CCC(=O)CCl)nn4)nn(C)c3c2)cc1Cl ; COc1ccc(-c2ccc3c(NC(=O)C(C)(C)COC(=O)CCl)nn(C)c3c2)cc1Cl ; COc1c(Cl)cc(-c2ccc3c(-n4cc(CCOC(=O)CCl)nn4)nn(C)c3c2)cc1Cl ; COc1ccc(-c2ccc3c(-n4cc(CCOC(=O)CCl)nn4)nn(C)c3c2)cc1Cl ; Cn1nc(-n2cc(CCOC(=O)CCl)nn2)c2ccc(-c3ccc(N4CCOCC4)c(Cl)c3)cc21 ; CC(C)Oc1ncc(-c2ccc3c(-n4cc(C(C)(C)COC(=O)CCl)nn4)nn(C)c3c2)cn1]

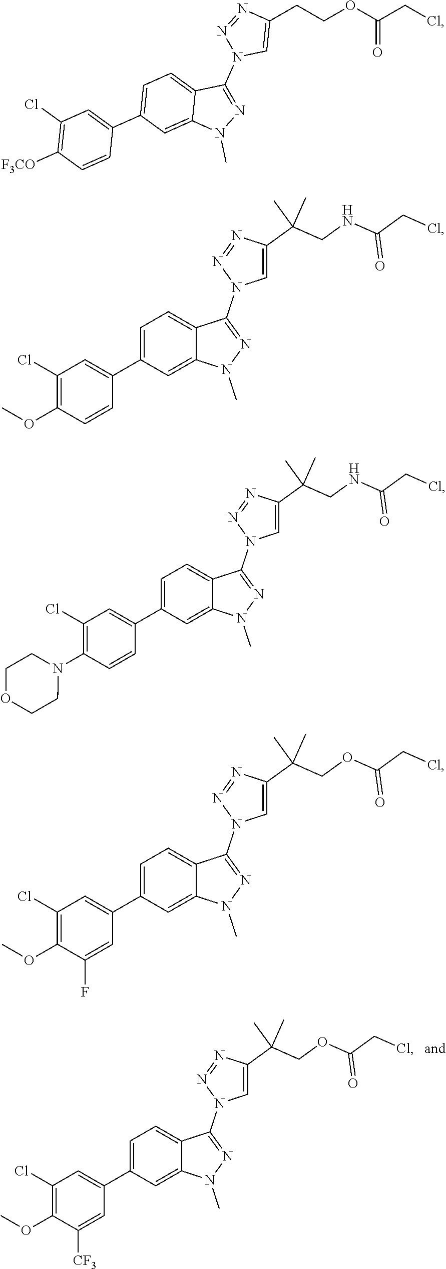 [SMILES: Cn1nc(-n2cc(CCOC(=O)CCl)nn2)c2ccc(-c3ccc(OC(F)(F)F)c(Cl)c3)cc21 ; COc1ccc(-c2ccc3c(-n4cc(C(C)(C)CNC(=O)CCl)nn4)nn(C)c3c2)cc1Cl ; Cn1nc(-n2cc(C(C)(C)CNC(=O)CCl)nn2)c2ccc(-c3ccc(N4CCOCC4)c(Cl)c3)cc21 ; COc1c(F)cc(-c2ccc3c(-n4cc(C(C)(C)COC(=O)CCl)nn4)nn(C)c3c2)cc1Cl ; COc1c(Cl)cc(-c2ccc3c(-n4cc(C(C)(C)COC(=O)CCl)nn4)nn(C)c3c2)cc1C(F)(F)F ; "and"]

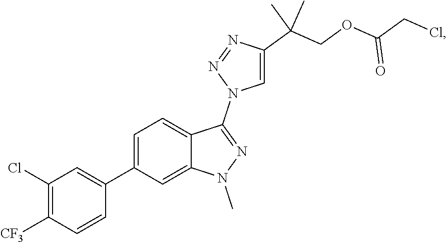 [SMILES: Cn1nc(-n2cc(C(C)(C)COC(=O)CCl)nn2)c2ccc(-c3ccc(C(F)(F)F)c(Cl)c3)cc21]

or an N-oxide, crystalline form, hydrate, or pharmaceutically acceptable salt thereof.

Another embodiment of the present disclosure is a compound according to formula (4):

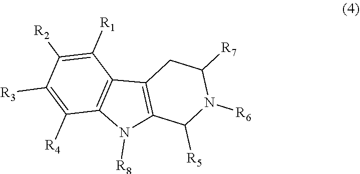

wherein:
R₁, R₂, R₃, R₄, R₅ and R₈ are independently selected from the group consisting of H, D, —OH, halo, ether, ester, furanyl, indole, indazole, pyrrole, pyrazole, pyridine, pyrimidine, naphthalene, indene, dibenzofuran, benzodioxole, amide, —(O)C(R), —C(O)OR, —NO₂, alkyl, aryl, alkyl-aryl, alkyl-heteroaryl, alkenyl, alkenyl-aryl, alkenyl-heteroaryl, peptide, and polypeptide, wherein the ether, ester, furanyl, indole, indazole, pyrrole, pyrazole, pyridine, pyrimidine, naphthalene, indene, dibenzofuran, benzodioxole, amide, alkyl, aryl, alkyl-aryl, alkyl-heteroaryl, alkenyl, alkenyl-aryl, alkenyl-heteroaryl, peptide, and polypeptide may be optionally substituted with an atom or a group selected from the group consisting of O, N, S, epoxy, —OH, halo, —CN, —NO₂, C₁₋₄alkyl, CF₃, —CF₃O, carbonyl, alkyl-aryl, thiol, methylmethanesulfonamide, methylthiol, dioxane, tetrahydropyzan, morpholinyl, piperazine, sulfonyl, oxazole, oxadiazole, dizaole, triazole, amide, ether, ester and combinations thereof; or R₁ and R₂ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₂ and R₃ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₃ and R₄ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof,
R₆ is selected from the group consisting of H, D, —OH, halo, C₁₋₄alkyl, CF₃, —(O)C(R), —C(O)OR, —NO₂, oxazole, oxadiazole, dizaole, triazole, amide, alcohol, ether, ester, and —C(O)C≡CSi(CH₃)₃,
R₇ is selected from the group consisting of H, D, —OH, halo, C₁₋₄alkyl, CF₃, —(O)C(R), —C(O)OR, —NO₂, oxazole, oxadiazole, dizaole, triazole, amide, alcohol, ether, and ester,
wherein R is selected from the group consisting of H, D, O, N, halo, oxazole, oxadiazole, dizaole, triazole, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of O, N, S, halo, C₁₋₄alkyl, CF₃, —CN and combinations thereof,
with the proviso that the compound is not

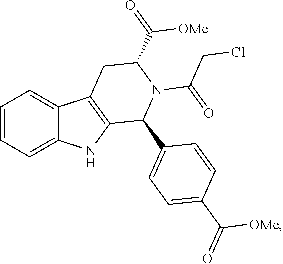 [SMILES: COC(=O)c1ccc([C@H]2c3[nH]c4ccccc4c3C[C@H](C(=O)OC)N2C(=O)CCl)cc1]

or an N-oxide, crystalline form, hydrate, or pharmaceutically acceptable salt thereof.

In some embodiments, the compound has a structure selected from the group consisting of:

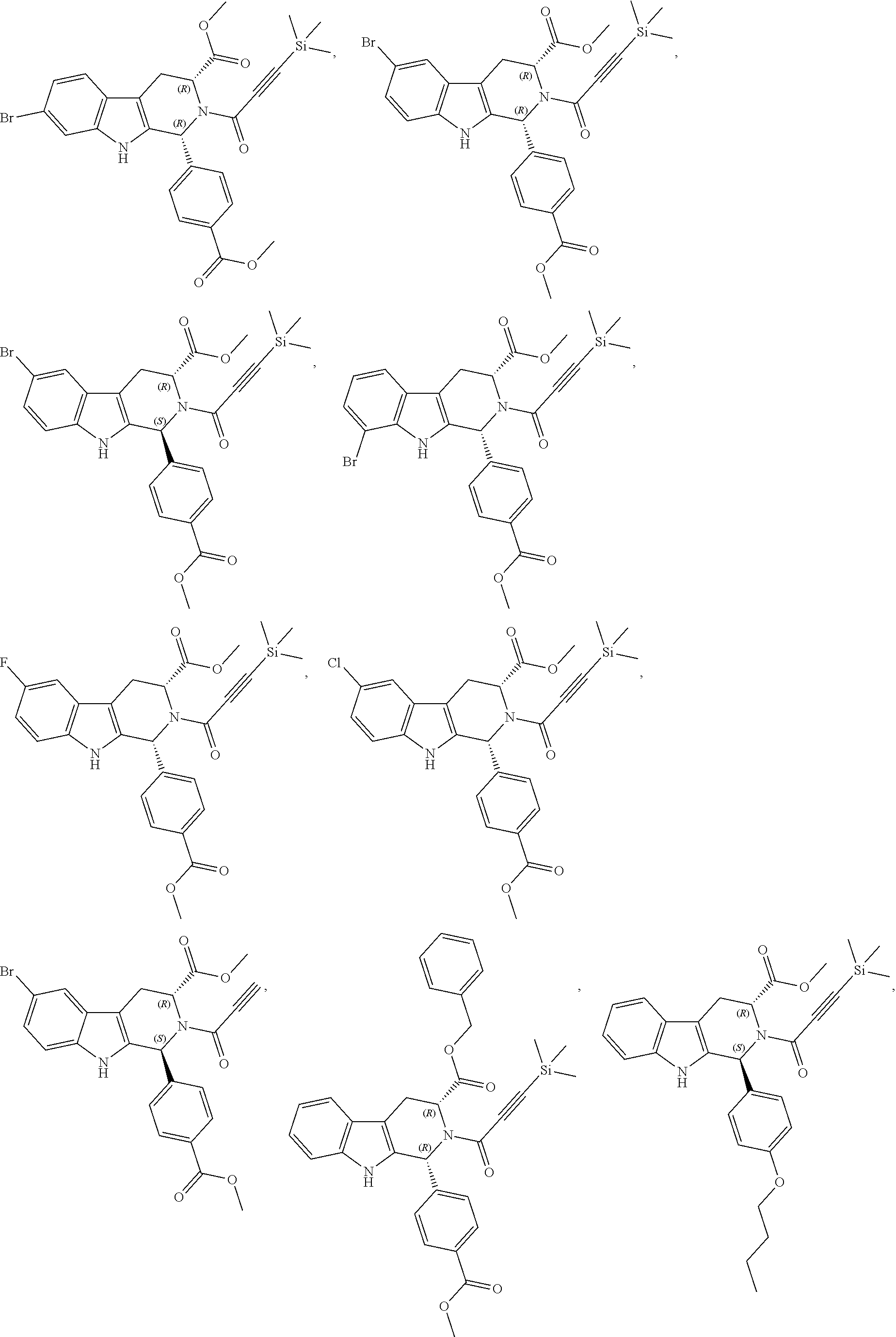

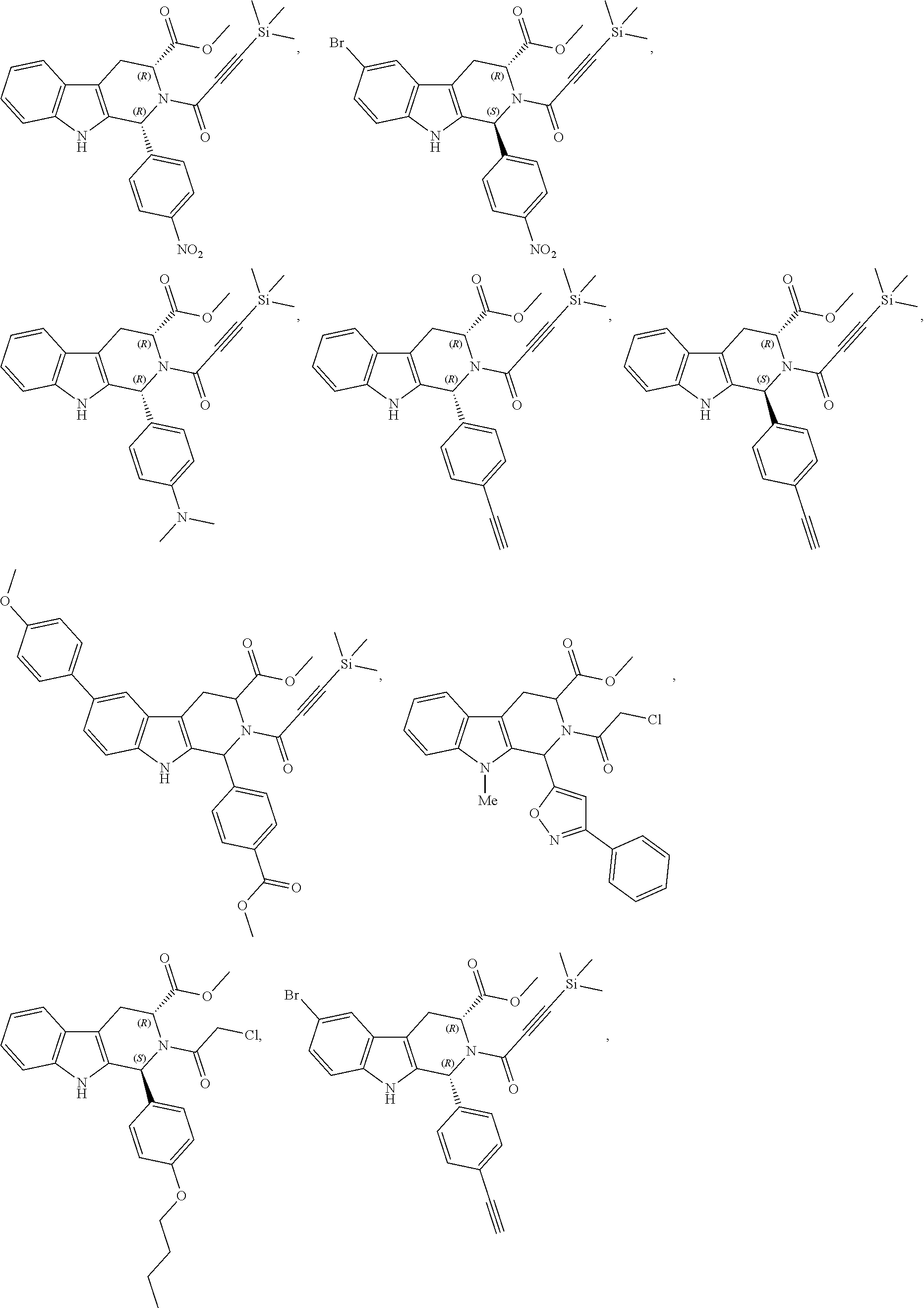

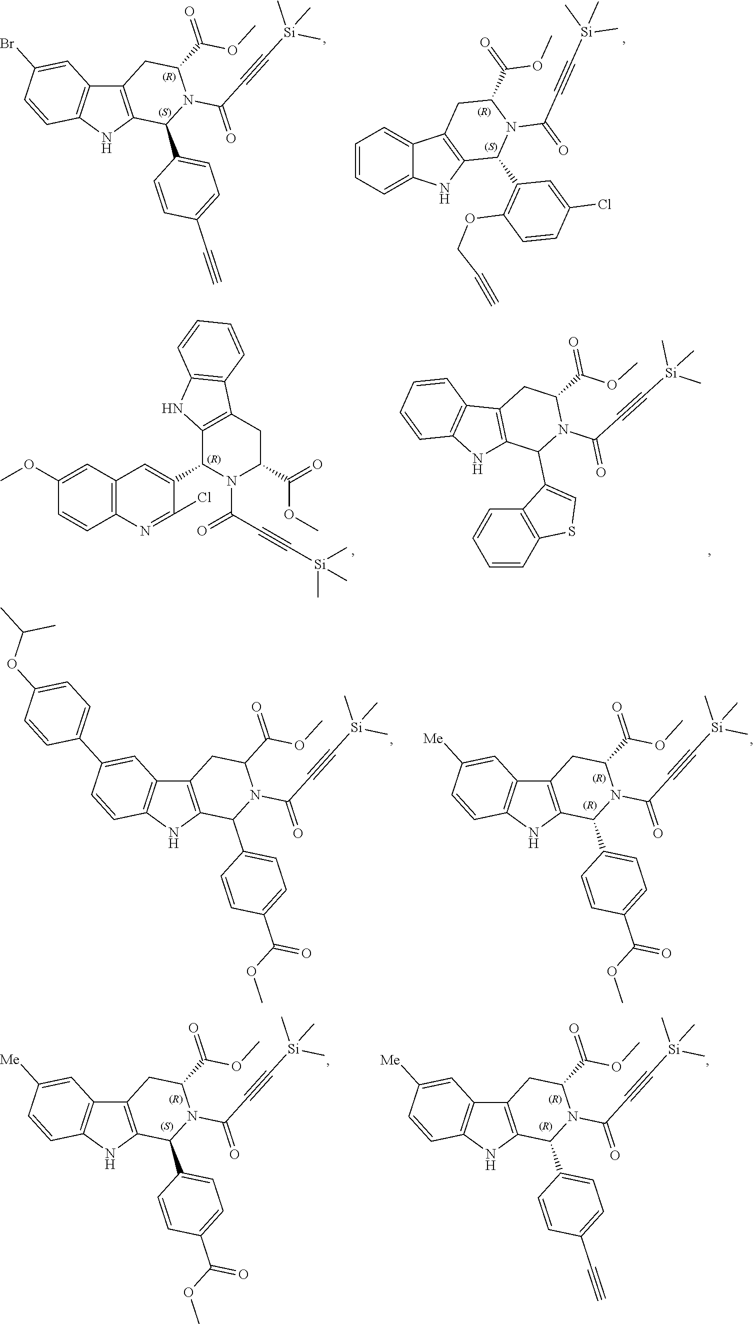

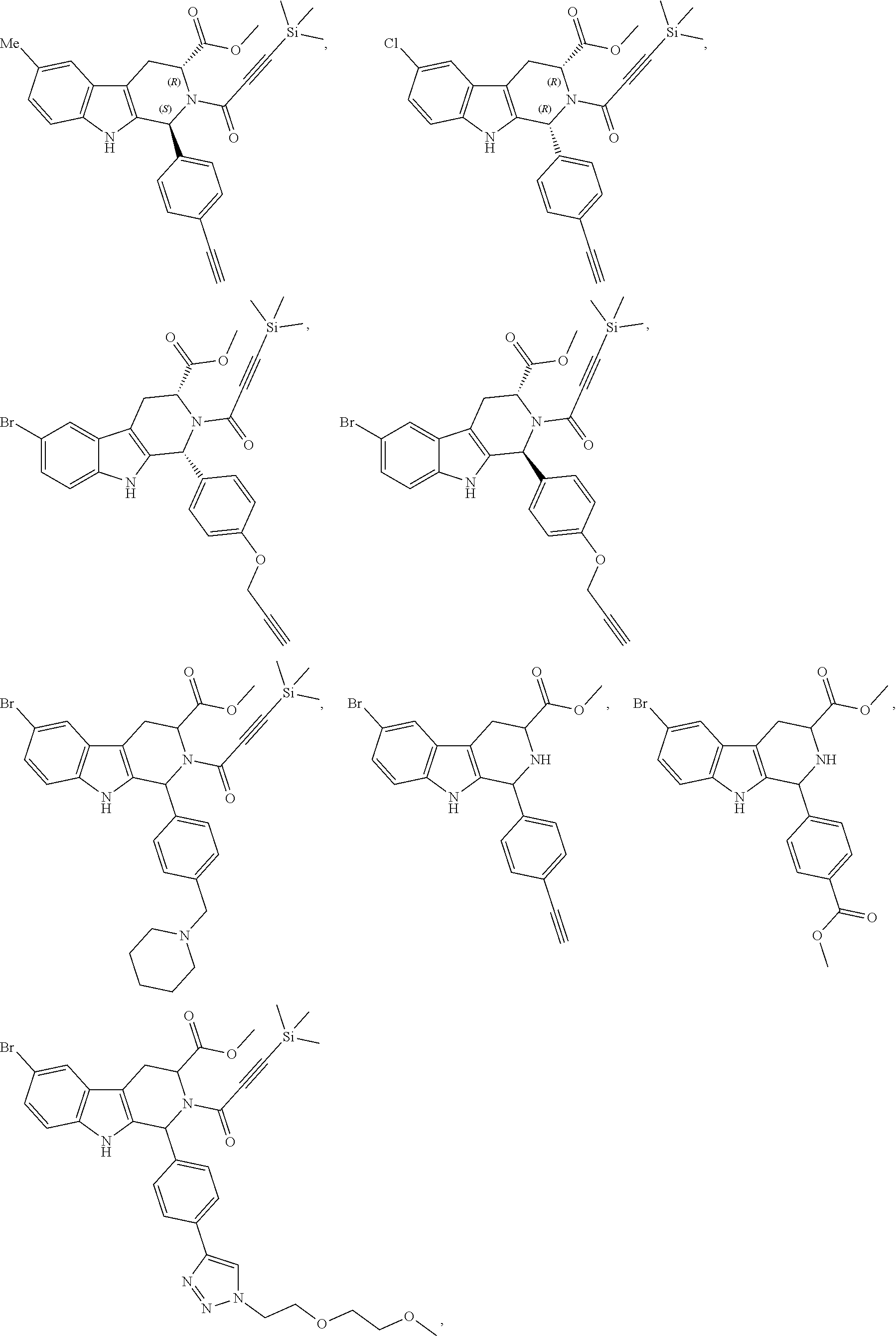

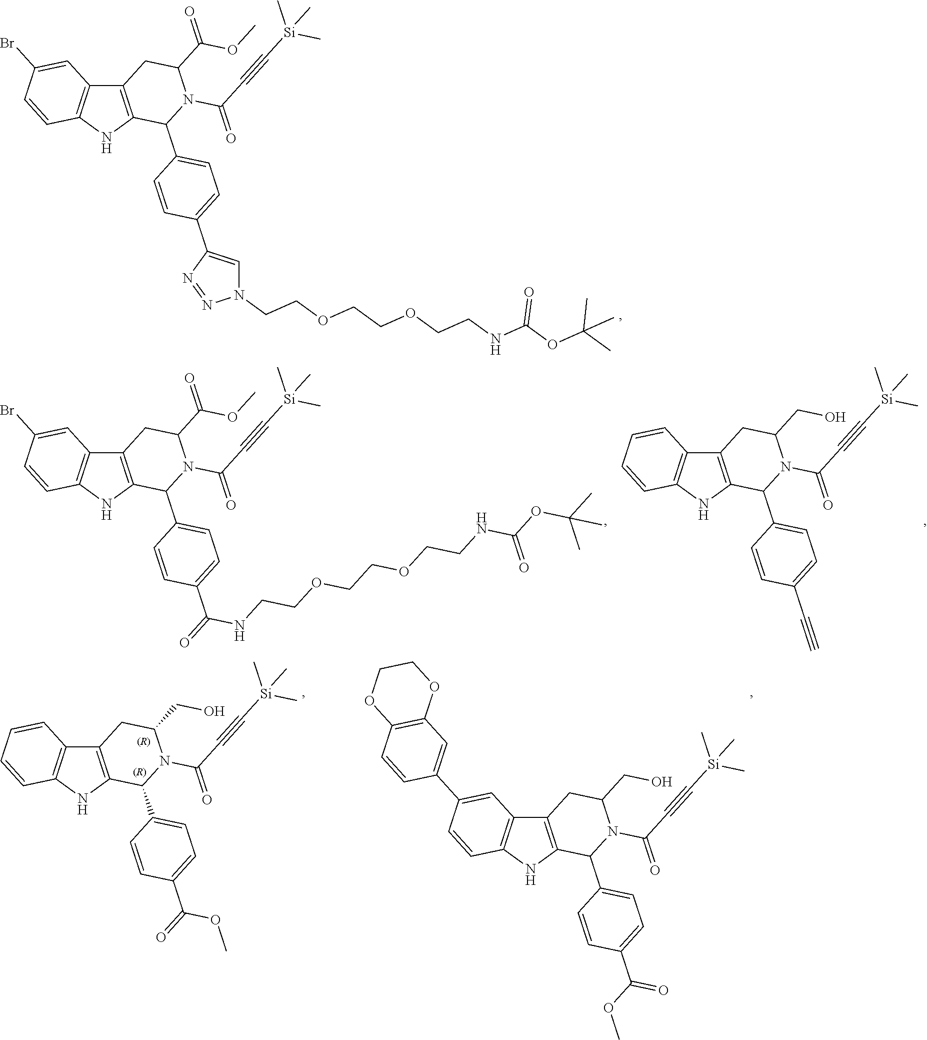

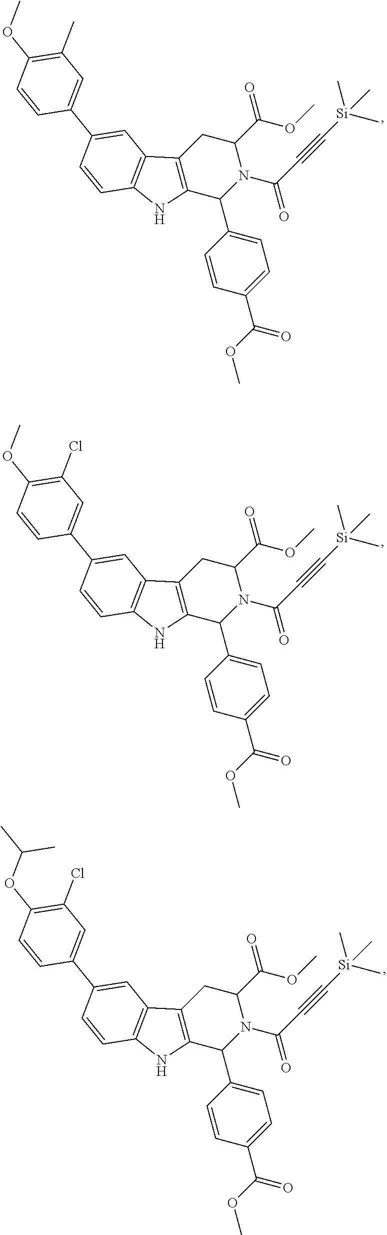

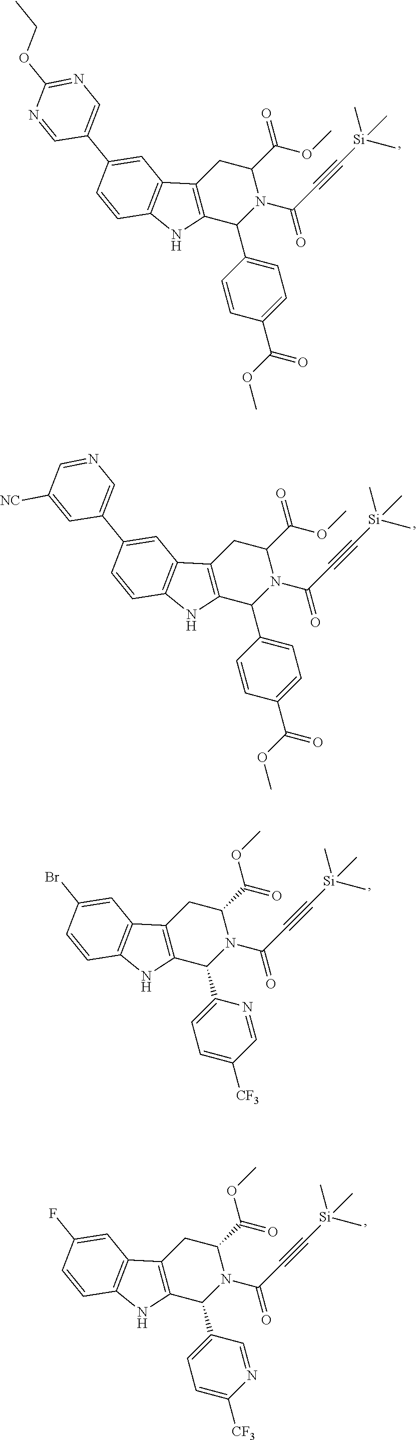

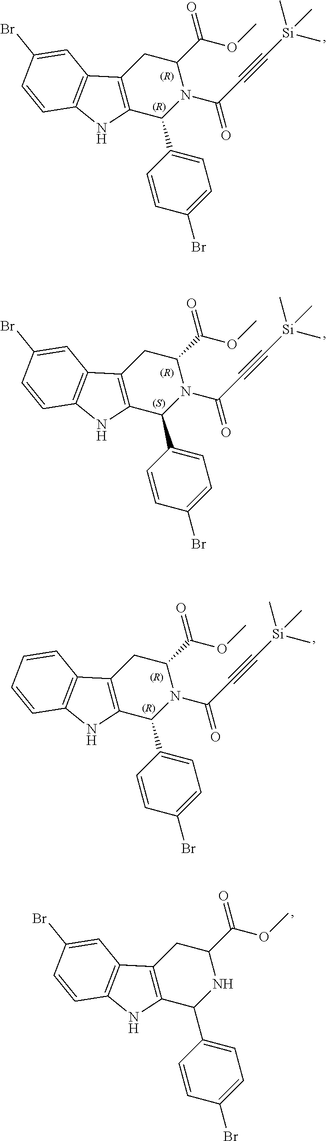

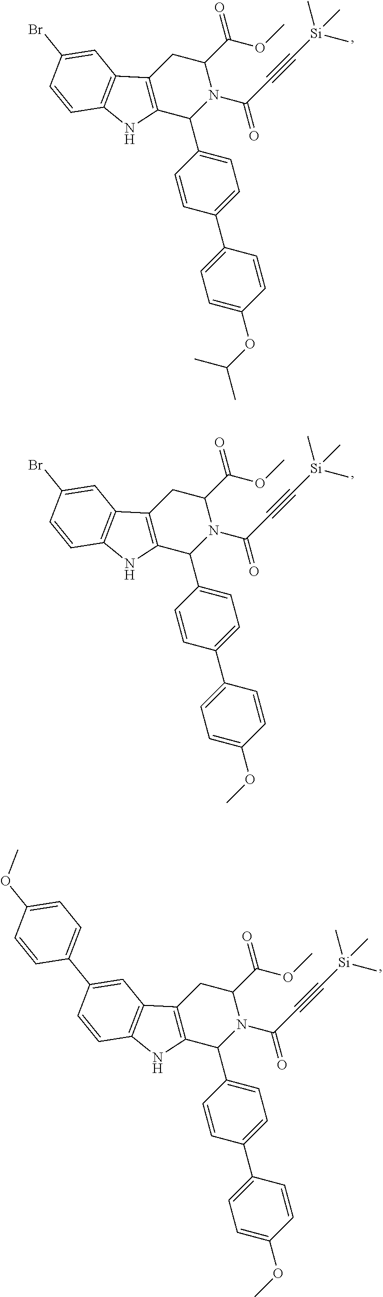

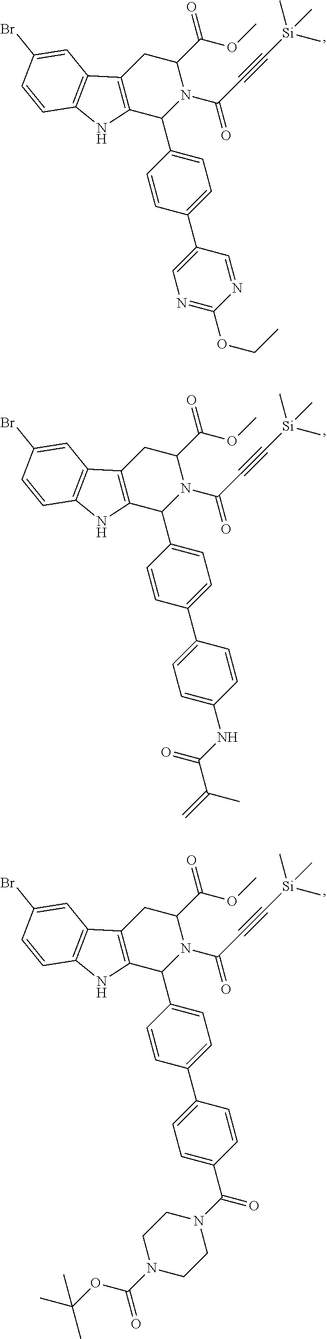

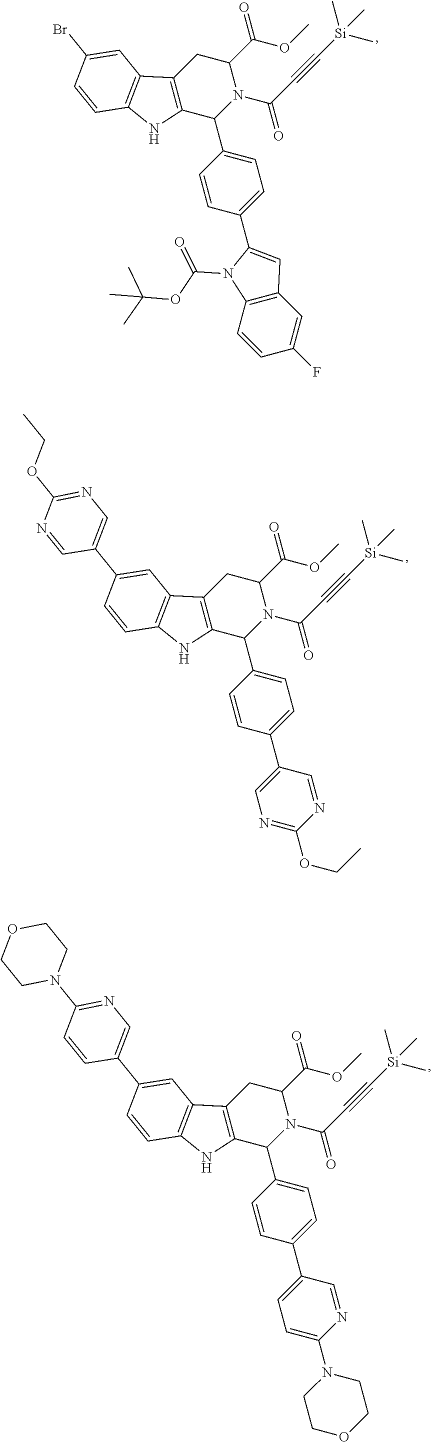

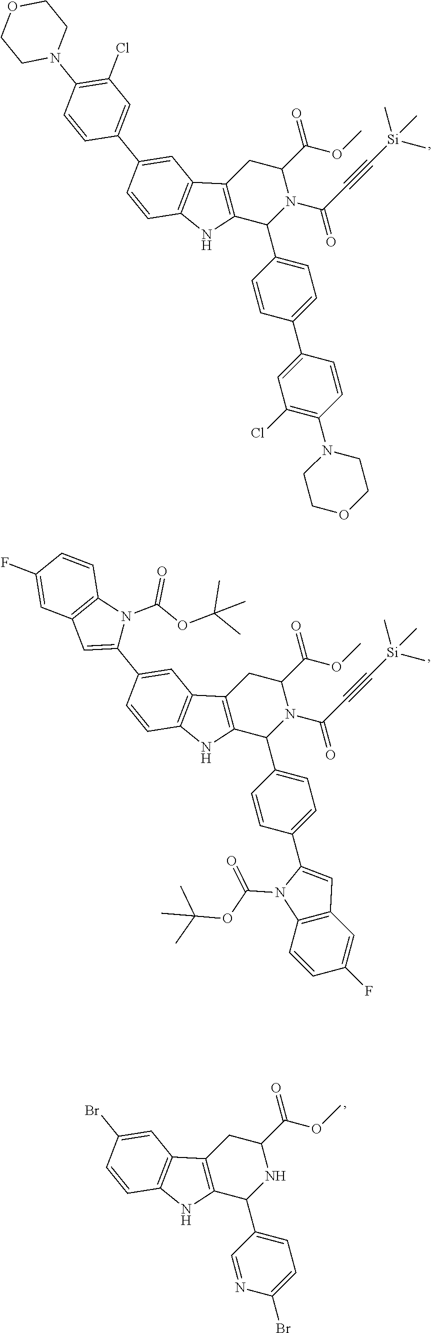 [SMILES: COC(=O)C1Cc2c([nH]c3ccc(-c4ccc(N5CCOCC5)c(Cl)c4)cc23)C(c2ccc(-c3ccc(N4CCOCC4)c(Cl)c3)cc2)N1C(=O)C#C[Si](C)(C)C ; COC(=O)C1Cc2c([nH]c3ccc(-c4cc5cc(F)ccc5n4C(=O)OC(C)(C)C)cc23)C(c2ccc(-c3cc4cc(F)ccc4n3C(=O)OC(C)(C)C)cc2)N1C(=O)C#C[Si](C)(C)C ; COC(=O)C1Cc2c([nH]c3ccc(Br)cc23)C(c2ccc(Br)nc2)N1]

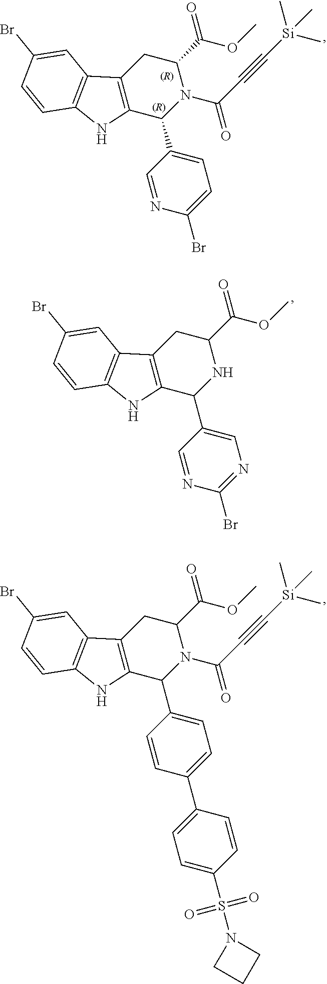

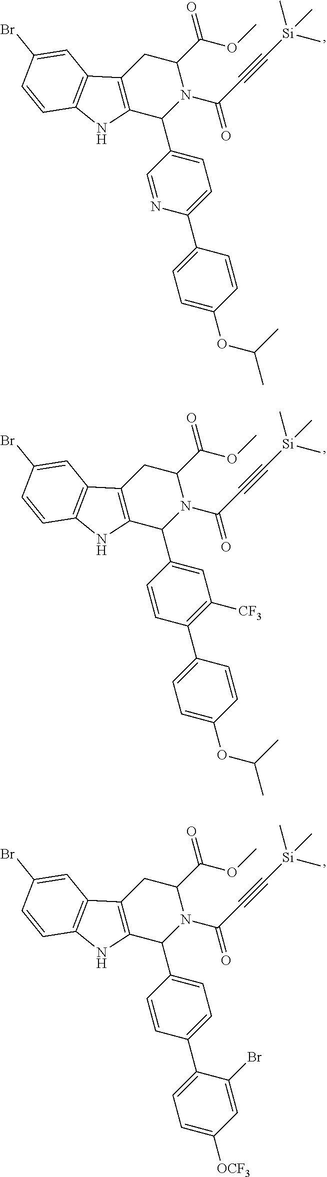

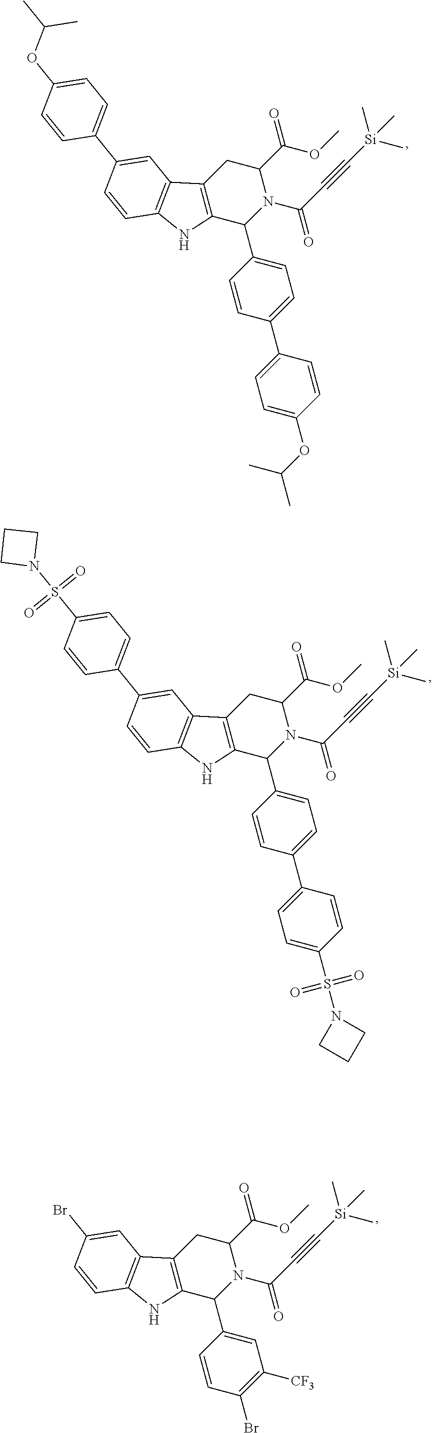 [SMILES: COC(=O)C1Cc2c([nH]c3ccc(-c4ccc(OC(C)C)cc4)cc23)C(c2ccc(-c3ccc(OC(C)C)cc3)cc2)N1C(=O)C#C[Si](C)(C)C ; COC(=O)C1Cc2c([nH]c3ccc(-c4ccc(S(=O)(=O)N5CCC5)cc4)cc23)C(c2ccc(-c3ccc(S(=O)(=O)N4CCC4)cc3)cc2)N1C(=O)C#C[Si](C)(C)C ; COC(=O)C1Cc2c([nH]c3ccc(Br)cc23)C(c2ccc(Br)c(C(F)(F)F)c2)N1C(=O)C#C[Si](C)(C)C]

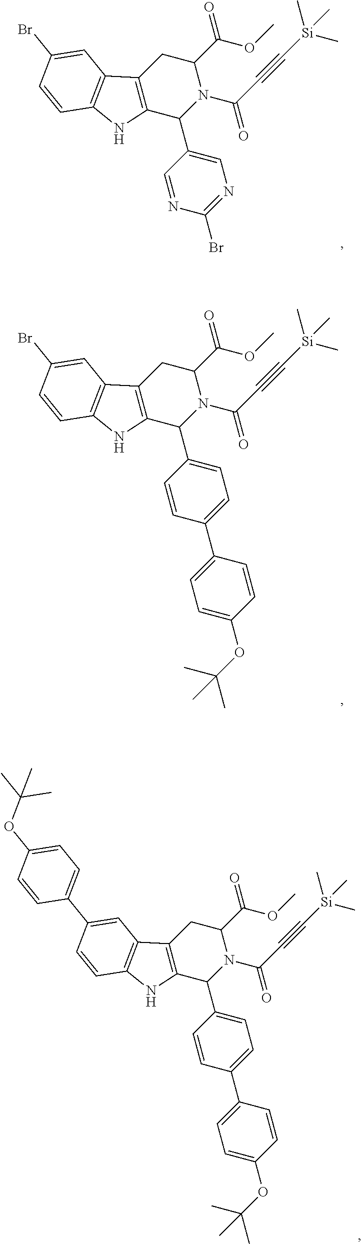

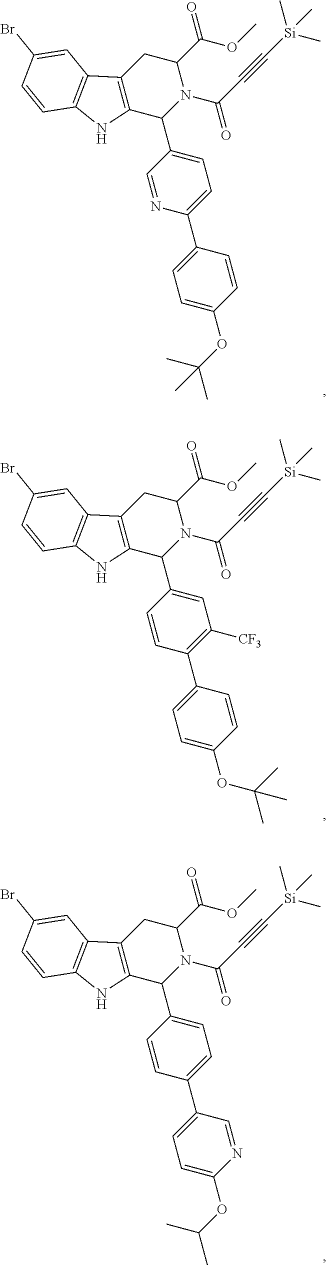

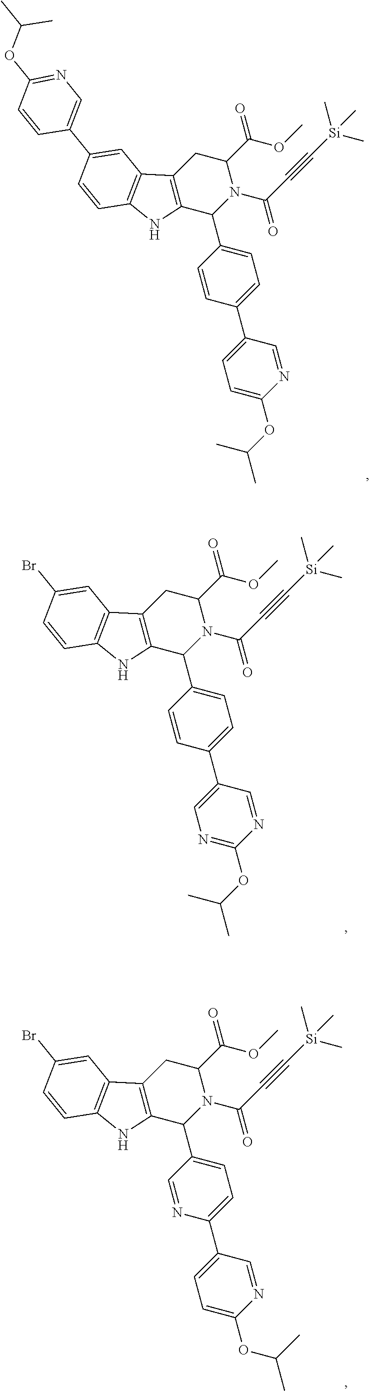 [SMILES: COC(=O)C1Cc2c([nH]c3ccc(-c4ccc(OC(C)C)nc4)cc23)C(c2ccc(-c3ccc(OC(C)C)nc3)cc2)N1C(=O)C#C[Si](C)(C)C ; COC(=O)C1Cc2c([nH]c3ccc(Br)cc23)C(c2ccc(-c3cnc(OC(C)C)nc3)cc2)N1C(=O)C#C[Si](C)(C)C ; COC(=O)C1Cc2c([nH]c3ccc(Br)cc23)C(c2ccc(-c3ccc(OC(C)C)nc3)nc2)N1C(=O)C#C[Si](C)(C)C]

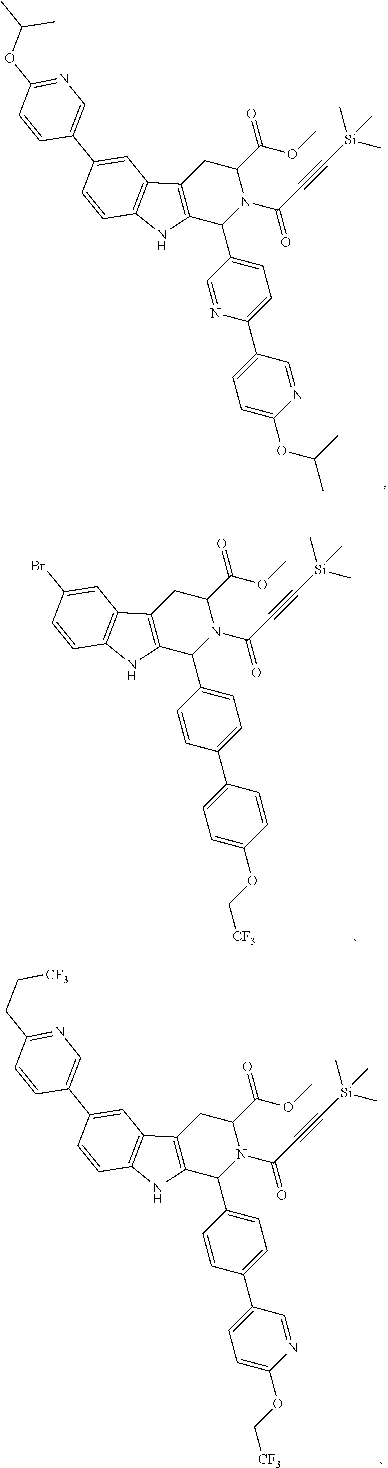 [SMILES: COC(=O)C1Cc2c([nH]c3ccc(-c4ccc(OC(C)C)nc4)cc23)C(c2ccc(-c3ccc(OC(C)C)nc3)nc2)N1C(=O)C#C[Si](C)(C)C ; COC(=O)C1Cc2c([nH]c3ccc(Br)cc23)C(c2ccc(-c3ccc(OCC(F)(F)F)cc3)cc2)N1C(=O)C#C[Si](C)(C)C ; COC(=O)C1Cc2c([nH]c3ccc(-c4ccc(CCC(F)(F)F)nc4)cc23)C(c2ccc(-c3ccc(OCC(F)(F)F)nc3)cc2)N1C(=O)C#C[Si](C)(C)C]

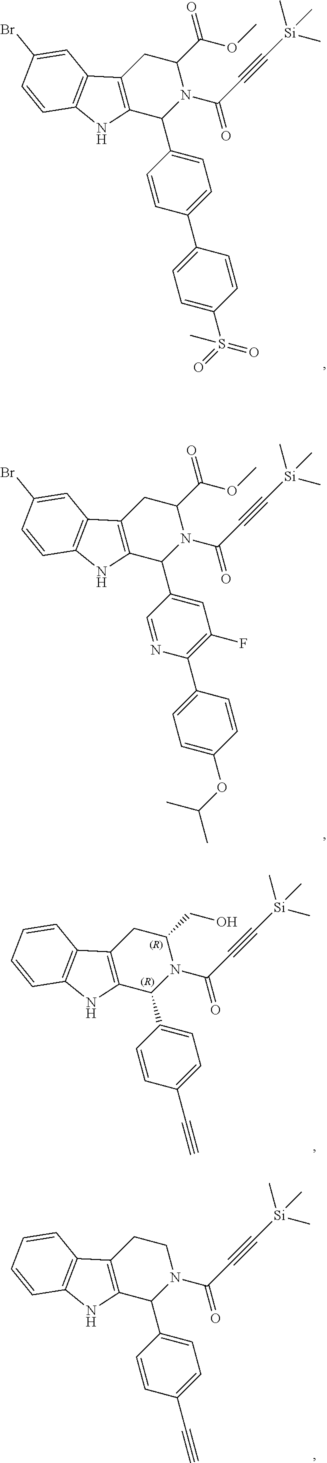

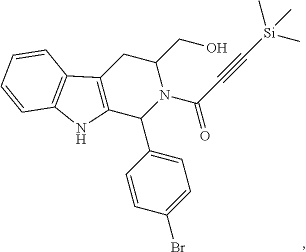 [SMILES: C[Si](C)(C)C#CC(=O)N1C(CO)Cc2c([nH]c3ccccc23)C1c1ccc(Br)cc1]

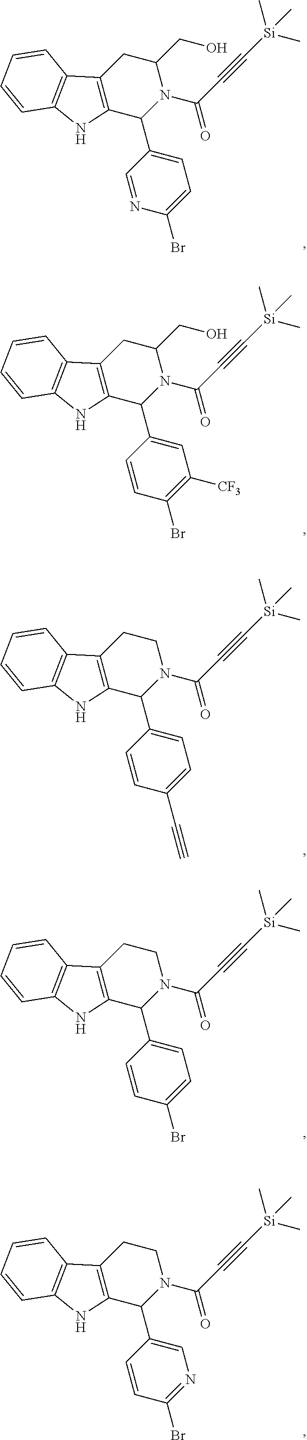 [SMILES: C[Si](C)(C)C#CC(=O)N1C(CO)Cc2c([nH]c3ccccc23)C1c1ccc(Br)nc1 ; C[Si](C)(C)C#CC(=O)N1C(CO)Cc2c([nH]c3ccccc23)C1c1ccc(Br)c(C(F)(F)F)c1 ; C#Cc1ccc(C2c3[nH]c4ccccc4c3CCN2C(=O)C#C[Si](C)(C)C)cc1 ; C[Si](C)(C)C#CC(=O)N1CCc2c([nH]c3ccccc23)C1c1ccc(Br)cc1 ; C[Si](C)(C)C#CC(=O)N1CCc2c([nH]c3ccccc23)C1c1ccc(Br)nc1]

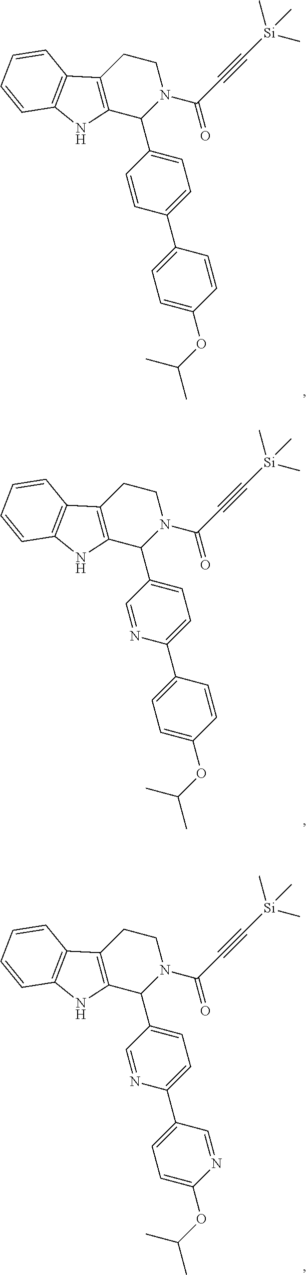 [SMILES: CC(C)Oc1ccc(-c2ccc(C3c4[nH]c5ccccc5c4CCN3C(=O)C#C[Si](C)(C)C)cc2)cc1 ; CC(C)Oc1ccc(-c2ccc(C3c4[nH]c5ccccc5c4CCN3C(=O)C#C[Si](C)(C)C)cn2)cc1 ; CC(C)Oc1ccc(-c2ccc(C3c4[nH]c5ccccc5c4CCN3C(=O)C#C[Si](C)(C)C)cn2)cn1]

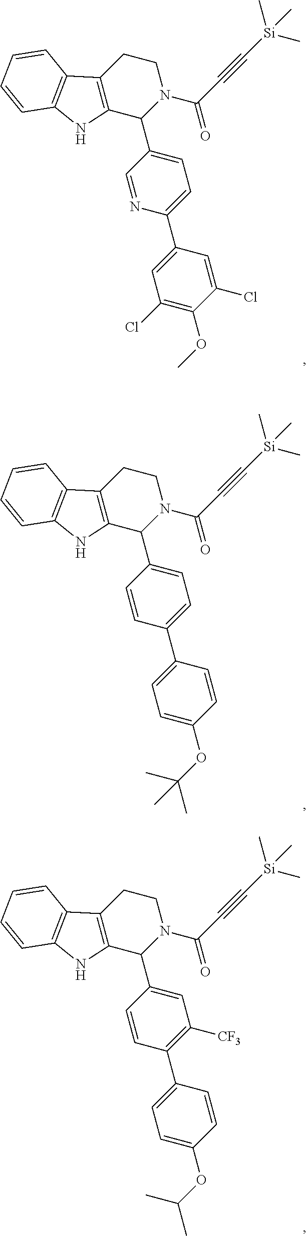 [SMILES: COc1c(Cl)cc(-c2ccc(C3c4[nH]c5ccccc5c4CCN3C(=O)C#C[Si](C)(C)C)cn2)cc1Cl ; CC(C)(C)Oc1ccc(-c2ccc(C3c4[nH]c5ccccc5c4CCN3C(=O)C#C[Si](C)(C)C)cc2)cc1 ; CC(C)Oc1ccc(-c2ccc(C3c4[nH]c5ccccc5c4CCN3C(=O)C#C[Si](C)(C)C)cc2C(F)(F)F)cc1]

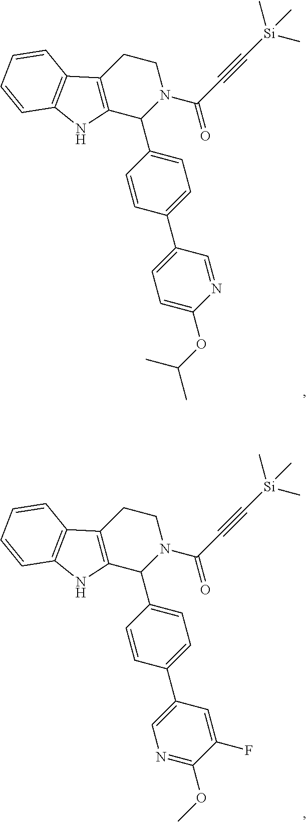 [SMILES: CC(C)Oc1ccc(-c2ccc(C3c4[nH]c5ccccc5c4CCN3C(=O)C#C[Si](C)(C)C)cc2)cn1 ; COc1ncc(-c2ccc(C3c4[nH]c5ccccc5c4CCN3C(=O)C#C[Si](C)(C)C)cc2)cc1F]

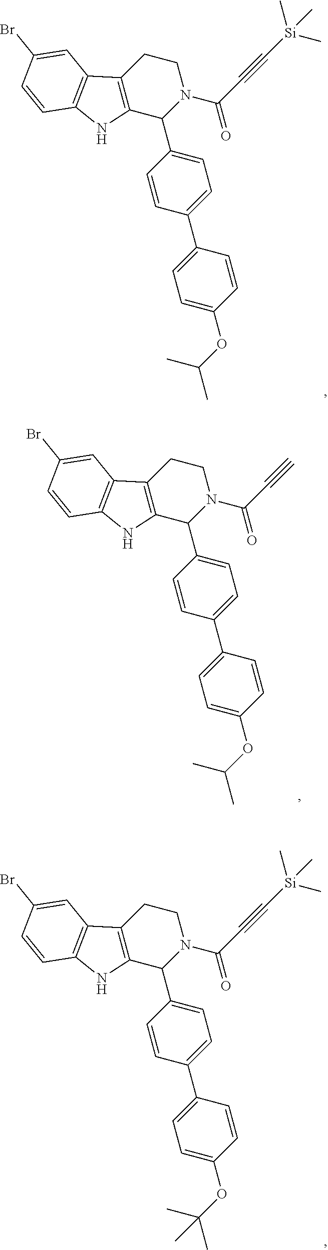 [SMILES: CC(C)Oc1ccc(-c2ccc(C3c4[nH]c5ccc(Br)cc5c4CCN3C(=O)C#C[Si](C)(C)C)cc2)cc1 ; C#CC(=O)N1CCc2c([nH]c3ccc(Br)cc23)C1c1ccc(-c2ccc(OC(C)C)cc2)cc1 ; CC(C)(C)Oc1ccc(-c2ccc(C3c4[nH]c5ccc(Br)cc5c4CCN3C(=O)C#C[Si](C)(C)C)cc2)cc1]

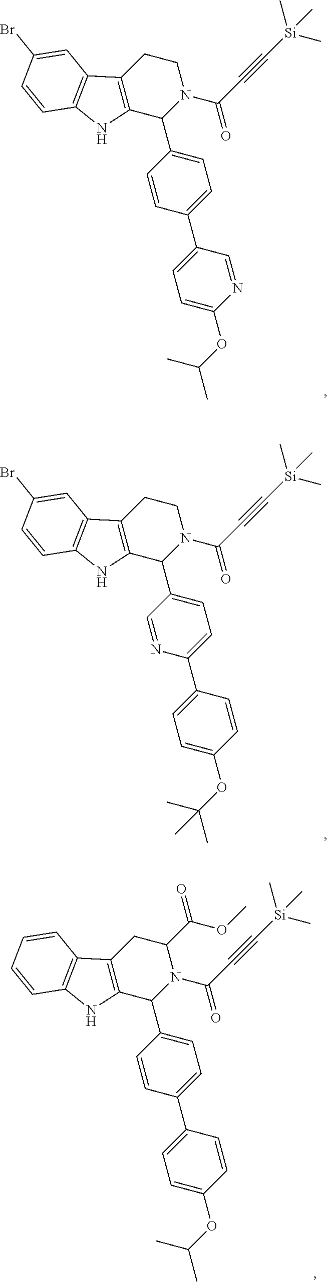 [SMILES: CC(C)Oc1ccc(-c2ccc(C3c4[nH]c5ccc(Br)cc5c4CCN3C(=O)C#C[Si](C)(C)C)cc2)cn1 ; CC(C)(C)Oc1ccc(-c2ccc(C3c4[nH]c5ccc(Br)cc5c4CCN3C(=O)C#C[Si](C)(C)C)cn2)cc1 ; COC(=O)C1Cc2c([nH]c3ccccc23)C(c2ccc(-c3ccc(OC(C)C)cc3)cc2)N1C(=O)C#C[Si](C)(C)C]

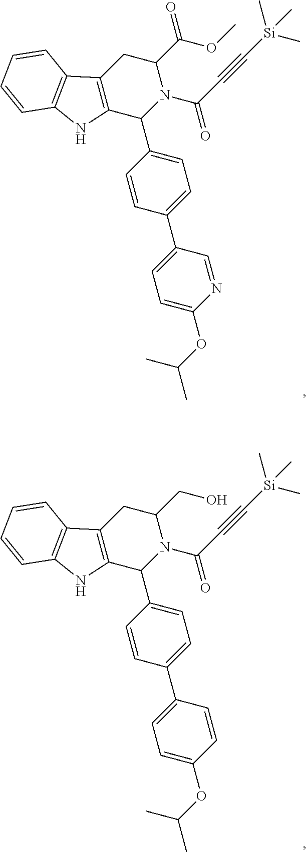 [SMILES: COC(=O)C1Cc2c([nH]c3ccccc23)C(c2ccc(-c3ccc(OC(C)C)nc3)cc2)N1C(=O)C#C[Si](C)(C)C ; CC(C)Oc1ccc(-c2ccc(C3c4[nH]c5ccccc5c4CC(CO)N3C(=O)C#C[Si](C)(C)C)cc2)cc1]

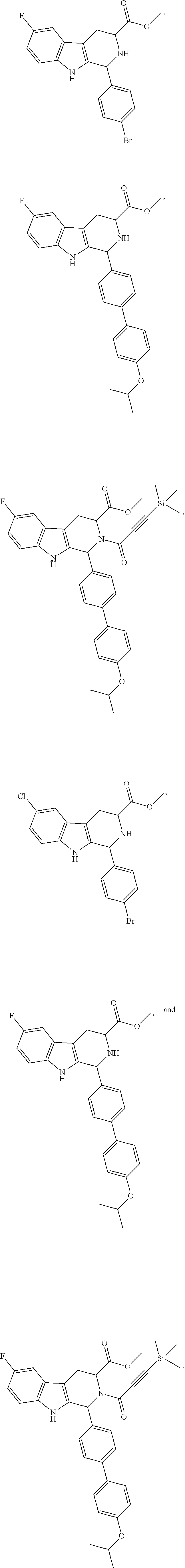 [SMILES: COC(=O)C1Cc2c([nH]c3ccc(F)cc23)C(c2ccc(Br)cc2)N1 ; COC(=O)C1Cc2c([nH]c3ccc(F)cc23)C(c2ccc(-c3ccc(OC(C)C)cc3)cc2)N1 ; COC(=O)C1Cc2c([nH]c3ccc(F)cc23)C(c2ccc(-c3ccc(OC(C)C)cc3)cc2)N1C(=O)C#C[Si](C)(C)C ; COC(=O)C1Cc2c([nH]c3ccc(Cl)cc23)C(c2ccc(Br)cc2)N1 ; COC(=O)C1Cc2c([nH]c3ccc(F)cc23)C(c2ccc(-c3ccc(OC(C)C)cc3)cc2)N1 ; COC(=O)C1Cc2c([nH]c3ccc(F)cc23)C(c2ccc(-c3ccc(OC(C)C)cc3)cc2)N1C(=O)C#C[Si](C)(C)C]

or an N-oxide, crystalline form, hydrate, or pharmaceutically acceptable salt thereof.

In some embodiments, the compound has a structure of:

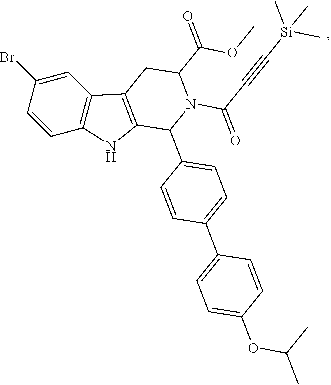 [SMILES: COC(=O)C1Cc2c([nH]c3ccc(Br)cc23)C(c2ccc(-c3ccc(OC(C)C)cc3)cc2)N1C(=O)C#C[Si](C)(C)C]

or an N-oxide, crystalline form, hydrate, or pharmaceutically acceptable salt thereof.

Another embodiment of the present disclosure is a compound according to formula (5):

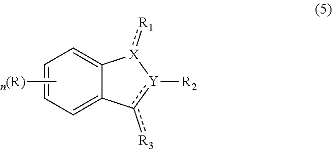

wherein:
a dashed line indicates the presence of an optional double bond;
n=2, 3, or 4;
X and Y are independently selected from the group consisting of C, N, S and O;
R₁, R₂, and R₃ are independently selected from the group consisting of no atom, H, D, O, N, halo, ether, ester, amide, C(O), (O)C(R), C(O) O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of N, O, Sn, halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not

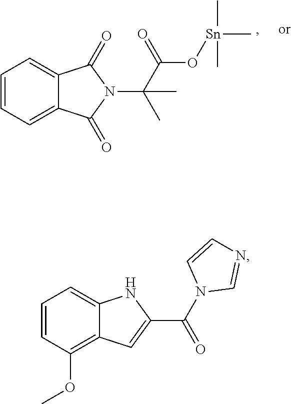 [SMILES: CC(C)(C(=O)[O][Sn]([CH3])([CH3])[CH3])N1C(=O)c2ccccc2C1=O ; COc1cccc2[nH]c(C(=O)n3ccnc3)cc12]

or an N-oxide, crystalline form, hydrate, or pharmaceutically acceptable salt thereof.

Another embodiment of the present disclosure is a composition, including pharmaceutical compositions, comprising one or more compounds disclosed herein and a pharmaceutically acceptable carrier, adjuvant or vehicle.

Another embodiment of the present disclosure is a method for treating or ameliorating the effects of a glutathione peroxidase 4 (GPX4)-associated disease in a subject in need thereof, comprising administering to the subject an effective amount of one or more compounds disclosed herein or the composition disclosed herein.

In some embodiments, the GPX4-associated disease is selected from the group consisting of a cancer, a neurotic disorder, a neurodegenerative disorder, spondylometaphyseal dysplasia, mixed cerebral palsy, pontocerebellar hypoplasia, and male infertility.

In some embodiments, the GPX4-associated disease is a cancer. Non-limiting examples of cancer include hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma, and non-small cell lung carcinoma. In some embodiments, the cancer is hepatocellular carcinoma.

In some embodiments, the subject is a mammal. In some embodiments, the mammal is selected from the group consisting of humans, veterinary animals, and agricultural animals. In some embodiments, the subject is a human.

In some embodiments, the cancer is metastatic. In some embodiments, the cancer is under epithelial-to-mesenchymal (EMT) transition. In some embodiments, the cancer is hypersensitive to ferroptosis and/or addicted to GPX4. In some embodiments, the cancer is refractory to standard cancer treatment. Non-limiting examples of standard cancer treatment include chemotherapy, radiation therapy, targeted therapy, immunotherapy, and combinations thereof.

Another embodiment of the present disclosure is a method for modulating the activity of glutathione peroxidase 4 (GPX4) in a subject in need thereof, comprising administering to the subject an effective amount of one or more compounds disclosed herein or the composition disclosed herein. In some embodiments, the modulation comprises inhibiting GPX4 activity.

Another embodiment of the present disclosure is a method for increasing the level of peroxide in a subject in need thereof, comprising administering to the subject an effective amount of one or more compounds disclosed herein or the composition disclosed herein. Non-limiting examples of peroxide include hydrogen peroxide, organic hydroperoxide, lipid peroxide, and combinations thereof.

A further embodiment of the present disclosure is a method for inducing ferroptosis in a cell, comprising contacting the cell with an effective amount of one or more compounds disclosed herein or the composition disclosed herein.

In some embodiments, the cell has aberrant lipid accumulation. In some embodiments, the cell is a cancer cell. In some embodiments, the cancer cell is selected from the group consisting of hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma, and non-small cell lung carcinoma. In some embodiments, the cancer is hepatocellular carcinoma.

In some embodiments, the cell is a human cell. In some embodiments, wherein the cancer cell is metastatic. In some embodiments, the cancer cell is under epithelial-to-mesenchymal (EMT) transition. In some embodiments, the cancer cell is hypersensitive to ferroptosis and/or addicted to GPX4. In some embodiments, the hypersensitivity to ferroptosis is identified by NADPH abundance, GCH1 expression, NF2-YAP activity, EMT signature, and GPX4 expression. In some embodiments, the cancer cell is refractory to standard cancer treatment. Non-limiting examples of standard cancer treatment includes chemotherapy, radiation therapy, targeted therapy, immunotherapy, and combinations thereof.

Another embodiment of the present disclosure is a method for treating or ameliorating the effects of a cancer in a subject in need thereof, comprising administering to the subject i) an effective amount of a first anti-cancer agent, which is one or more compounds disclosed herein or the composition disclosed herein, and ii) an effective amount of a second anti-cancer agent.

In some embodiments, the second anti-cancer agent is selected from the group consisting of chemotherapy, radiation therapy, targeted therapy, immunotherapy, and combinations thereof. In some embodiments, the second anti-cancer agent is an immunotherapy, such as checkpoint inhibitor therapy including PD-1 and CTLA-4 inhibitor therapy. Non-limiting examples of immunotherapy include ipilimumab, pembrolizumab, nivolumab, atezolizumab, avelumab, durvalumab, cemiplimab, ofatumumab, blinatumomab, daratumumab, elotuzumab, obinutuzumab, talimogene laherparepvec, necitumumab, lenalidomide, dinutuximab, and combinations thereof.

In some embodiments, the subject is a human.

In some embodiments, the cancer is metastatic. In some embodiments, the cancer is under epithelial-to-mesenchymal (EMT) transition. In some embodiments, the cancer is hypersensitive to ferroptosis and/or addicted to GPX4. In some embodiments, the cancer is refractory to standard cancer treatment.

In some embodiments, the cancer is selected from the group consisting of hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma, and non-small cell lung carcinoma. In some embodiments, the cancer is hepatocellular carcinoma.

In some embodiments, the first anti-cancer agent is administered to the subject before, concurrently with, or after the second anti-cancer agent.

An additional embodiment of the present disclosure is a kit for treating or ameliorating the effects of a glutathione peroxidase 4 (GPX4)-associated disease in a subject in need thereof, comprising an effective amount of one or more compounds disclosed herein or the composition disclosed herein, packaged with its instructions for use.

The kits may also include suitable storage containers, e.g., ampules, vials, tubes, etc., for each compound of the present disclosure (which, e.g., may be in the form of pharmaceutical compositions) and other reagents, e.g., buffers, balanced salt solutions, etc., for use in administering the active agents to subjects. The compounds and/or pharmaceutical compositions of the disclosure and other reagents may be present in the kits in any convenient form, such as, e.g., in a solution or in a powder form. The kits may further include a packaging container, optionally having one or more partitions for housing the compounds and/or pharmaceutical compositions and other optional reagents.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present disclosure may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

As used herein, a “subject” is a mammal, preferably, a human. In addition to humans, categories of mammals within the scope of the present disclosure include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc. in the context of the present disclosure, the phrase “a subject in need thereof” means a subject in need of treatment for a GPX4-associated disorder, such as, e.g., a cancer. Alternatively, the phrase “a subject in need thereof” means a subject diagnosed with a GPX4-associated disorder, such as, e.g., a cancer.

As used herein, “lipid peroxidation” means the oxidative degradation of fats, oils, waxes, sterols, triglycerides, and the like. Lipid peroxidation has been linked with many degenerative diseases, such as atherosclerosis, ischemia-reperfusion, heart failure, Alzheimer's disease, rheumatic arthritis, cancer, and other immunological disorders. (Ramana et al., 2013).

As used herein, “ferroptosis” means regulated cell death that is iron-dependent. Ferroptosis is characterized by the overwhelming, iron-dependent accumulation of lethal lipid reactive oxygen species. (Dixon et al., 2012) Ferroptosis is distinct from apoptosis, necrosis, and autophagy. (Id.)

As used herein, the terms “modulate”, “modulating”, “modulator” and grammatical variations thereof mean to change, such as increasing or decreasing the activity of GPX4. In this embodiment, “contacting” means bringing the compound and optionally one or more additional therapeutic agents into close proximity to the cells in need of such modulation. This may be accomplished using conventional techniques of drug delivery to the subject or in the in vitro situation by, e.g., providing the compound and optionally other therapeutic agents to a culture media in which the cells are located.

As used herein, a “pharmaceutically acceptable salt” means a salt of the compounds of the present disclosure which are pharmaceutically acceptable, as defined herein, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

In the present disclosure, an “effective amount” or “therapeutically effective amount” of a compound or pharmaceutical composition is an amount of such a compound or composition that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of a compound or pharmaceutical composition according to the disclosure will be that amount of the compound or composition, which is the lowest dose effective to produce the desired effect with no or minimal side effects. The effective dose of a compound or pharmaceutical composition according to the present disclosure may be administered as two, three, four, five, six or more sub-doses, administered separately at appropriate intervals throughout the day.

A suitable, non-limiting example of a dosage of a compound or pharmaceutical composition according to the present disclosure or a composition comprising such a compound, is from about 1 ng/kg to about 1000 mg/kg, such as from about 1 mg/kg to about 100 mg/kg, including from about 5 mg/kg to about 50 mg/kg. Other representative dosages of a compound or a pharmaceutical composition of the present disclosure include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, or 1000 mg/kg.

A compound, composition, or pharmaceutical composition of the present disclosure may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, a compound, composition, or pharmaceutical composition of the present disclosure may be administered in conjunction with other treatments. A compound, composition, or pharmaceutical composition of the present disclosure may be encapsulated or otherwise protected against gastric or other secretions, if desired.

The compositions or pharmaceutical compositions of the disclosure are pharmaceutically acceptable and comprise one or more active ingredients in admixture with one or more pharmaceutically acceptable carriers or diluents and, optionally, one or more other compounds, drugs, ingredients and/or materials. Regardless of the route of administration selected, the compounds/compositions/pharmaceutical compositions of the present disclosure are formulated into pharmaceutically acceptable dosage forms by conventional methods known to those of skill in the art. See, e.g., Remington, The Science and Practice of Pharmacy (21^{st} Edition, Lippincott Williams and Wilkins, Philadelphia, PA.). More generally, “pharmaceutically acceptable” means that which is useful in preparing a composition that is generally safe, non-toxic, and neither biologically nor otherwise undesirable and includes that which is acceptable for veterinary use as well as human pharmaceutical use.

Pharmaceutically acceptable carriers and diluents are well known in the art (see, e.g., Remington, The Science and Practice of Pharmacy (21^{st} Edition, Lippincott Williams and Wilkins, Philadelphia, PA.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, salicylate, etc. Each pharmaceutically acceptable carrier or diluent used in a composition of the disclosure must be “acceptable” in the sense of being compatible with the other ingredients of the formulation and not injurious to the subject. Carriers or diluents suitable for a selected dosage form and intended route of administration are well known in the art, and acceptable carriers or diluents for a chosen dosage form and method of administration can be determined using ordinary skill in the art.

The compositions or pharmaceutical compositions of the disclosure may, optionally, contain additional ingredients and/or materials commonly used in such compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Compounds, compositions or pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically acceptable carriers or diluents and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Compositions for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Compositions which are suitable for vaginal administration also include pessaries, tampons, creams, gels, pastes, foams or spray formulations containing such pharmaceutically acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically acceptable carrier or diluent. The ointments, pastes, creams and gels may contain excipients. Powders and sprays may contain excipients and propellants.

Compositions suitable for parenteral administrations comprise one or more agent(s)/compound(s) in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier or diluent, for example water for injection, immediately prior to use. Extemporaneous injection solutions and suspensions may be prepared from sterile powders, granules and tablets of the type described above.

In the foregoing embodiments, the following definitions apply.

The term “aliphatic”, as used herein, refers to a group composed of carbon and hydrogen that do not contain aromatic rings. Accordingly, aliphatic groups include alkyl, alkenyl, alkynyl, and carbocyclyl groups. Additionally, unless otherwise indicated, the term “aliphatic” is intended to include both “unsubstituted aliphatics” and “substituted aliphatics”, the latter of which refers to aliphatic moieties having substituents replacing a hydrogen on one or more carbons of the aliphatic group. Such substituents can include, for example, a halogen, a deuterium, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic, or heteroaromatic moiety.

The term “alkyl” refers to the radical of saturated aliphatic groups that does not have a ring structure, including straight-chain alkyl groups, and branched-chain alkyl groups. In certain embodiments, a straight chain or branched chain alkyl has 6 or fewer carbon atoms in its backbone (e.g., C₁-C₆ for straight chains, C₃-C₆ for branched chains). In other embodiments, the “alkyl” may include up to twelve carbon atoms, e.g., C₁, C₂, C₃, C₄, C₅, C₆, C₇, C₈, C₉, C₁₀, C₁₁ or C₁₂. Such substituents include all those contemplated for aliphatic groups, as discussed below, except where stability is prohibitive.

The term “alkenyl”, as used herein, refers to an aliphatic group containing at least one double bond and unless otherwise indicated, is intended to include both “unsubstituted alkenyls” and “substituted alkenyls”, the latter of which refers to alkenyl moieties having substituents replacing a hydrogen on one or more carbons of the alkenyl group. Such substituents include all those contemplated for aliphatic groups, as discussed below, except where stability is prohibitive. For example, substitution of alkenyl groups by one or more alkyl, carbocyclyl, aryl, heterocyclyl, or heteroaryl groups is contemplated.

Moreover, unless otherwise indicated, the term “alkyl” as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Indeed, unless otherwise indicated, all groups recited herein are intended to include both substituted and unsubstituted options.

The term “C_{x-y}” when used in conjunction with a chemical moiety, such as, alkyl and cycloalkyl, is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc.

The term “aryl” as used herein includes substituted or unsubstituted single-ring aromatic groups in which each atom of the ring is carbon. Preferably the ring is a 3- to 8-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “alkyl-aryl” refers to an alkyl group substituted with at least one aryl group.

The term “alkyl-heteroaryl” refers to an alkyl group substituted with at least one heteroaryl group.

The term “alkenyl-aryl” refers to an alkenyl group substituted with at least one aryl group.

The term “alkenyl-heteroaryl” refers to an alkenyl group substituted with at least one heteroaryl group.

The terms “carbocycle”, “carbocyclyl”, and “carbocyclic”, as used herein, refer to a non-aromatic saturated or unsaturated ring in which each atom of the ring is carbon, preferably a carbocycle ring contains from 3 to 10 atoms, more preferably from 3 to 8 atoms, including 5 to 7 atoms, such as for example, 6 atoms. The term “carbocycle” also includes bicycles, tricycles and other multicyclic ring systems, including the adamantyl ring system.

The terms “halo” and “halogen” are used interchangeably herein and mean halogen and include chloro, fluoro, bromo, and iodo.

The term “heteroaryl” includes substituted or unsubstituted aromatic single ring structures, preferably 3- to 8-membered rings, more preferably 5- to 7-membered rings, even more preferably 5- to 6-membered rings, whose ring structures include at least one heteroatom, preferably one to four heteroatoms, more preferably one or two heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, and the like.

The term “heteroatom” as used herein means an atom of any element other than carbon or hydrogen. Preferred heteroatoms are nitrogen, oxygen, and sulfur; more preferably, nitrogen and oxygen.

The term “ketone” means an organic compound with the structure RC(═O)R′, wherein neither R nor R′ can be hydrogen atoms.

The term “ether” means an organic compound with the structure R—O—R′, wherein neither R nor R′ can be hydrogen atoms.

The term “ester” means an organic compound with the structure RC(═O)OR′, wherein neither R nor R′ can be hydrogen atoms.

The term “polyyne” means is an organic compound with alternating single and triple bonds; that is, a series of consecutive alkynes, (—C≡C—) n with n greater than 1.

The term “substituted” refers to moieties having substituents replacing a hydrogen on one or more carbons of the backbone. It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

As set forth previously, unless specifically stated as “unsubstituted,” references to chemical moieties herein are understood to include substituted variants. For example, reference to an “aryl” group or moiety implicitly includes both substituted and unsubstituted variants.

As used herein, the term “furan” or “furanyl” means any compound or chemical group containing the following structure or positional isomers thereof:

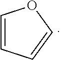

As used herein, the term “oxadiazole” means any compound or chemical group containing the following structure or positional isomers thereof:

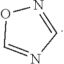

As used herein, the term “oxazole” means any compound or chemical group containing the following structure or positional isomers thereof:

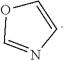

As used herein, the term “triazole” means any compound or chemical group containing the following structure or positional isomers thereof:

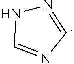

As used herein, the term “indole” means any compound or chemical group containing the following structure or positional isomers thereof:

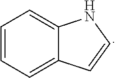

As used herein, the term “indazole” means any compound or chemical group containing the following structure or positional isomers thereof:

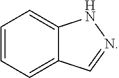

As used herein, the term “pyrrole” means any compound or chemical group containing the following structure or positional isomers thereof:

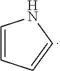

As used herein, the term “pyrazole” means any compound or chemical group containing the following structure or positional isomers thereof:

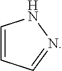

As used herein, the term “pyridine” means any compound or chemical group containing the following structure or positional isomers thereof:

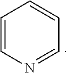

As used herein, the term “pyrimidine” means any compound or chemical group containing the following structure or positional isomers thereof:

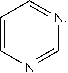

As used herein, the term “naphthalene” means any compound or chemical group containing the following structure:

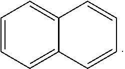

As used herein, the term “indene” means any compound or chemical group containing the following structure:

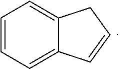

As used herein, the term “dibenzofuran” means any compound or chemical group containing the following structure:

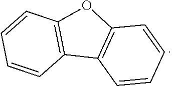 [SMILES: c1ccc2c(c1)oc1ccccc12]

As used herein, the term “dioxane” means any compound or chemical group containing the following structure or positional isomers thereof:

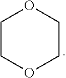

As used herein, the term “tetrahydropyzan” means any compound or chemical group containing the following structure:

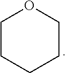

As used herein, the term “morpholine” or “morpholinyl” means any compound or chemical group containing the following structure or positional isomers thereof:

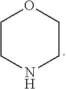

As used herein, the term “piperazine” means any compound or chemical group containing the following structure or positional isomers thereof:

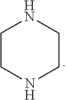

As used herein, the term “benzodioxole” means any compound or chemical group containing the following structure or positional isomers thereof:

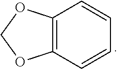

It is understood that the disclosure of a compound herein encompasses all stereoisomers of that compound. As used herein, the term “stereoisomer” refers to a compound made up of the same atoms bonded by the same bonds but having different three-dimensional structures which are not interchangeable. The three-dimensional structures are called configurations. Stereoisomers include enantiomers and diastereomers.

The terms “racemate” or “racemic mixture” refer to a mixture of equal parts of enantiomers. The term “chiral center” refers to a carbon atom to which four different groups are attached. The term “enantiomeric enrichment” as used herein refers to the increase in the amount of one enantiomer as compared to the other.

It is appreciated that to the extent compounds of the present disclosure have a chiral center, they may exist in and be isolated in optically active and racemic forms. Some compounds may exhibit polymorphism. It is to be understood that the present disclosure encompasses any racemic, optically-active, diastereomeric, polymorphic, or stereoisomeric form, or mixtures thereof, of a compound of the disclosure, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

Examples of methods to obtain optically active materials are known in the art, and include at least the following:
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts as disclosed in more detail herein or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

The stereoisomers may also be separated by usual techniques known to those skilled in the art including fractional crystallization of the bases or their salts or chromatographic techniques such as LC or flash chromatography. The (+) enantiomer can be separated from the (−) enantiomer using techniques and procedures well known in the art, such as that described by J. Jacques, et al., Enantiomers, Racemates, and Resolutions”, John Wiley and Sons, Inc., 1981. For example, chiral chromatography with a suitable organic solvent, such as ethanol/acetonitrile and Chiralpak AD packing, 20 micron can also be utilized to effect separation of the enantiomers.

The following examples are provided to further illustrate the methods of the present disclosure. These examples are illustrative only and are not intended to limit the scope of the disclosure in any way.

### EXAMPLES

### Example 1

### Methods and Materials

### Expression and Purification of GPX4^{U46C} Protein

The bacterial expression vector pET-15b-His-tagged-c-GPX4^{U46C} was prepared as previously described (Yang et al. 2016). The C-terminal His-tagged GPX4U46C protein was expressed in E. coli and purified according to a protocol modified from Scheerer et al. 2007. First, pET-15b-His-tagged-c-GPX4^{U46C} was transformed into XL10-Gold (Agilent #200314) E. coli ultracompetent cells for plasmid DNA production and isolated using QIAprep Spin Miniprep Kit (Qiagen). For protein expression, pET-15b-His-tagged-c-GPX4^{U46C} was then transformed into BL21-Gold (DE3) E. coli competent cells (Agilent #230132) and plated on LB agar with 100 μg/mL ampicillin. A starter culture was inoculated in 7 mL of LB medium with 100 μg/mL ampicillin using a single colony and allowed to grow in a 37° C. shaker (225 rpm) for 16 hours. 3 mL of the starter culture was added to 1 L of LB medium with 100 μg/mL ampicillin, which was then incubated in a 37° C. shaker (225 rpm) to an OD600 of 0.9. At that point, the incubation temperature was reduced to 15° C. and the culture was allowed to equilibrate for 1 hour. Expression of the recombinant enzyme was induced by addition of isopropyl β-D-1-thiogalactopyranoside (IPTG, 1 mM final concentration) overnight for 12-15 hours shaking at 225 rpm and 15° C. The next morning, cells were harvested by centrifugation at 4000 rpm and 4° C. for 20 minutes. The cell pellet was then frozen at −80° C. for at least 1 hour or until lysis. To lyse the cells, the cell pellet was resuspended with 25 mL of chilled lysis buffer (100 mM Tris pH 8.0, 300 mM NaCl, 20 mM imidazole, 3 mM TCEP, and 2.5 mini tablets of complete Protease Inhibitor Cocktail (Roche-Sigma #11836170001)) then lysed using an Emulsiflex C3 high-pressure homogenizer. To remove cell debris, the lysate was subjected to centrifugation at 10000 rpm and 4° C. for 20 minutes. The supernatant was centrifuged once more at the same conditions, then the clarified lysate was applied onto a 5-mL HisTrap HP column (Cytiva #17-5248-01), washed with 90% Buffer A (100 mM Tris pH 8.0, 300 mM NaCl, 5% glycerol, 3 mM TCEP) 10% Buffer B (Buffer A with 500 mM imidazole), then eluted with a continuous gradient (10-100% Buffer B). The protein was concentrated using Amicon Ultra-15 centrifugal filter units with a 10 kDa molecule weight cutoff (EMD Millipore #UFC901024) then further purified by FPLC on a size exclusion HiLoad Superdex 200 column (Cytiva) in crystallization buffer (20 mM Tris pH 8.0, 300 mM NaCl, 3 mM TCEP). The fractions containing 90-95% pure GPX4 protein, as evaluated by SDS-PAGE, were pooled and concentrated using the same Amicon centrifugal filter units as above to 5 mg/mL. Concentration was determined by Nanodrop. Protein was aliquoted into Eppendorf tubes, flash frozen using liquid nitrogen, and stored at −80° C. until use.

### Microscale Thermophoresis (MST)

MST experiments were conducted using a Monolith NT.115 (Nanotemper Technologies) according to manufacturer instructions. To label the protein, 90 μL of 200 nM GPX4^{U46C} was combined with 90 μL of 100 nM RED-tris-NTA dye diluted in PBS buffer with 0.05% Tween 20 (PBST buffer). The mixture was incubated for 30 minutes at room temperature, followed by centrifugation for 10 minutes at 4° C. and 15,000×g. To assess binding of the compounds, a 16-point 2-fold dilution series in PBST buffer was prepared and mixed in a 1:1 ratio with the labeled protein solution for a final volume of 20 μL. The reaction mixture was loaded into standard treated capillary tubes and analyzed with the Monolith NT.115 at 40% LED power and 40% MST power with a laser-on time of 5 seconds. The data were analyzed in MO.Affinity Analysis v2.3 software.

### Intact Protein MALDI MS Analysis

50 μM GPX4^{U46C} was pre-incubated with 375 μM LOC1886, or 1.5% DMSO vehicle control in buffer (20 mM Tris pH 9.0, 100 mM NaCl, 2 mM TCEP) at RT for 1 hour before transferring to 15° C. overnight. The next, day 1 μL of the apoprotein (pre-incubated with DMSO) or protein-inhibitor complex (pre-incubated with the inhibitor of interest) was mixed with 9 μL of 10 mg/mL sinapinic acid in matrix solution consisting of 70:30 water: acetonitrile with 0.1% TFA. 1.0 μL of the final mix was applied to the target carrier and air dried. MALDI spectra were recorded using a Bruker ultrafleXtreme MALDI-TOF instrument. The molecular weight of the target protein was used to set the range of m/z detection and suppression. The laser was set to 2000 Hz and 50% intensity. Five spectra were collected for each sample and the sum was recorded for analysis. Mass shifts were determined by comparing the MALDI spectrum of the protein-inhibitor complex with that of the apoprotein. A mass shift corresponding to the potential staying group of the inhibitor was indicative of covalent binding.

### Native Size Exclusion Chromatography-ESI-MS (SEC-MS) Analysis (Non-Denaturing)

50 μM GPX4U4C was pre-incubated with 375 μM LOC1886, or 1.5% DMSO vehicle control in buffer (20 mM Tris pH 9.0, 300 mM NaCl, 2 mM TCEP) at RT for 1 hour before transferring to 15° C. overnight. The next day, native protein SEC-MS analysis was used to evaluate binding of noncovalent inhibitors to GPX4^{U46C}. This analysis was performed on a Waters I-Class Plus UPLC system connected to a RDa mass detector (BioAccord system). A Waters Acquity UPLC Protein BEH SEC (2.1×150 mm, 1.7 μm, 200 Å) column was used with isocratic flow of 50 mM ammonium acetate pH 6.8 and a flow rate of 0.1 mL/min. The Rda mass detector was set to a cone voltage of 30 V, capillary voltage of 1.5 kV, and desolvation temperature of 350° C. MS deconvolution was completed through UNIFI using MaxEnt 1.

### Protein Crystallography

GPX4^{U46C} was pre-incubated with covalent inhibitors before crystallization using conditions that were optimized with monitoring on intact protein MALDI MS analysis. 40 μM GPX4^{U46C} was incubated with 375 μM LOC1886 in the reaction buffer (20 mM Tris pH 9.0, 100 mM NaCl, 2 mM TCEP, 1.5% DMSO) at 37° C. for 4 hours and then 4° C. overnight. Covalent binding was confirmed using intact protein MALDI MS analysis. The protein-inhibitor complex was then exchanged into crystallization buffer (20 mM Tris, pH 8.0, 300 mM NaCl, 3 mM TCEP) and concentrated to 5 mg/mL.

Protein samples of GPX4^{U46C} in complex with LOC1886 were initially screened at the High-Throughput Crystallization Screening Center of the Hauptman-Woodward Medical Research Institute (HWI) (hwi.buffalo.edu/high-throughput-crystallization-center/) (Luft et al. 2003). The most promising crystal hits were reproduced using under oil micro batch method at 23° C. Crystals of GPX4^{U46C} with LOC1886 were grown using crystallization reagent comprising 2 M sodium chloride and 0.1 M sodium acetate trihydrate, pH 4.6, then transferred into crystallization reagent supplemented with 20% (v/v) ethylene glycol before being flash frozen in liquid nitrogen.

GPX4^{U46C} with LOC1886 diffracted X-ray at the beamline NE-CAT24-ID-C to resolution 1.93 Å. The images were processed and scaled in space group C222₁ using XDS (Kabsch, 2010). The structure of each protein was determined by molecular replacement method using MOLREP program and the crystal structures of GPX4^{U46C} (PDB id: 7L8K) and GPX4^{U46C-R152H} (PDB id: 7L8L) were both used as search models for structure determination (Vagin and Teplyakov, 2010). The geometry of each crystal was fixed using programs XtalView and COOT, and refined by Phenix (McRee, 1999; Emsley et al. 2010; Adams et al. 2010). The asymmetric unit (ASU) of each crystal contained one protomer for GPX4^{U46C} with LOC1886.

All figures depicting crystal structures and surface potential were produced using PyMOL (pymol.org/2/) with the APBS plug-in (Baker et al. 2001).

¹H, ¹⁵N-HSQC-NMR spectrum for ¹⁵N isotope-Labeled GPX4^{U46C}

Uniformly 15N-labeled GPX4^{U46C} protein with an N-terminal His₆ tag was prepared using a protocol adapted from Feng et al. 2019. The GPX4^{U46C} construct was expressed in BL21-Gold (DE3) E. coli (Stratagene) cultured at 37° C. in M9 minimal medium supplemented with 100 μg/mL ampicillin, 2 mM MgSO4, 100 mM CaCl₂, 1× trace metals, 1×RPMI-1640 vitamin solution (Sigma-Aldrich #R7256), 10 mg/mL biotin, 10 mg/mL thiamine hydrochloride, 4 g/L glucose, and 3 g/L ¹⁵NH₄Cl as the sole nitrogen source. The subsequent induction, lysis, and protein purification were the same as that described above for the non-isotope-labeled His-tagged GPX4^{U46C}, except buffer exchange was performed to remove the imidazole after the His-Trap purification, then 5U/mg thrombin was added to remove the N-terminal His₆ tag and the reaction was incubated overnight at 4° C. before purification by FPLC.

For ¹H, ¹⁵N-HSQC-NMR analysis of GPX4^{U46C} with inhibitors, 50 μM ¹⁵N-labeled GPX4 was preincubated with 800 μM inhibitor to be tested for 6 hours at room temperature in buffer consisting of 100 mM MES, 5 mM TCEP, pH 6.5. D2O (10%) was then added for the field frequency lock. The ¹H, ¹⁵N-HSQC-NMR spectra were collected on a Bruker Avance III 500 Ascend (500 MHz) spectrometer (Columbia University) at 298K. The ¹H carrier frequency was positioned at the water resonance. The ¹⁵N carrier frequency was positioned at 115 ppm. The spectral width was 7,500 Hz in the ¹H dimension was 7,500 Hz and 1,824.6 in the ¹⁵N dimension. Suppression of water signal was accomplished using the WATERGATE sequence. Heteronuclear decoupling was accomplished using the GARP decoupling scheme.

### Cell Lines and Cell Culture

HT-1080 (human [Homo sapiens] male fibrosarcoma), A-673 (human [Homo sapiens] female Ewing's sarcoma), and SK-HEP-1 (human [Homo sapiens] male hepatic adenocarcinoma), cells were obtained from ATCC. Huh7 (human [Homo sapiens] male hepatocellular carcinoma), HLF (human [Homo sapiens] male hepatocellular carcinoma), HepG2 (human [Homo sapiens] male hepatocellular carcinoma), cells were obtained from the Cancer Cell Bank at Columbia University Irving Medical Center. HT-1080, A-673, Huh-7, and HLF cells were grown in Dulbecco's Modified Eagle Medium (DMEM) supplemented with 10% heat inactivated fetal bovine serum (HI-FBS), 1% non-essential amino acids, and 1% penicillin streptomycin. HepG2 and SK-HEP-1 cells were grown in Eagle's Minimum Essential Medium (EMEM) supplemented with 10% HI-FBS, and 1% penicillin-streptomycin. All cells were cultured at 37° C. and 5% CO₂.

### Cellular Dose Response Assay

HT-1080, HepG2, Huh7, HLF, and SK-HEP-1 cells were seeded into opaque white 384-well plates at 1500 cells per well then incubated at 37° C. with 5% CO₂ overnight. The next day, compounds of interest were dissolved in DMSO and 12-point 2-fold dilution series were prepared with and without Fer-1. Cells were treated in triplicate (final DMSO concentration of 0.4% in all wells, including DMSO-only control wells) and incubated for a further 48 hours at 37° C. and 5% CO₂. Cell viability was evaluated using CellTiter-Glo (Promega G7573) and luminescence was recorded on a Victor 5 plate reader. Data were analyzed in GraphPad Prism 9 and error bars represent standard deviation values for three technical replicates in a representative experiment.

### Western Blot Assay

HT-1080 cells were seeded at 250,000 cells per well in a 6-well plate and incubated 37° C. with 5% CO₂ overnight. The next day, cells were treated with vehicle alone or the compound of interest with 10 μM Fer-1 for 10 hours. Compounds were dissolved in DMSO. Cells were harvested with trypsin, washed with cold PBS, and lysed in RIPA buffer containing complete Protease Inhibitor Cocktail mini tablets (Roche) on ice for 30 minutes. Samples were then centrifuged at 14,000×g for 15 minutes at 4° C. Supernatants were collected and protein concentrations were determined using the BCA Protein Assay Kit (Pierce). Concentrations were normalized across samples and mixed with 6×SDS-PAGE Sample Loading Buffer (G Biosciences). Protein samples were then loaded on a NuPAGE Novex 4-12% Bis-Tris protein gel and run in MES buffer (Invitrogen) at 100V for 10 minutes then 200V for an additional 30 minutes. Proteins were transferred to a PVDF membrane (Invitrogen) using the iBlot 2 Gel Transfer Device. The membrane was washed with PBS, then blocked using Intercept Blocking Buffer (LICOR) for one hour. The membrane was then incubated with the primary antibodies in a 1:1 solution of blocking buffer and PBST (PBS+0.1% Tween 20) overnight at 4° C. The next day, the membrane was washed three times with PBST, incubated with secondary antibodies in a 1:1 solution of blocking buffer and PBST, then washed another three times with PBST. The primary antibodies used were GPX4 (Abcam, ab125066, 1:250 dilution) and β-actin (Cell Signaling, 8H10D10, 1:3000 dilution). The blot was imaged using a LI-COR Odyssey CLx IR scanner and results were quantified in ImageJ and GraphPad Prism 9.

### GPX4 Activity Assay in Cell Lysates

Determination of GPX4 activity in cell lysates was performed as previously described by Roveri et al. 1994 with minor modifications. Briefly, the assay measures the consumption of NADPH, which has a characteristic absorbance at 340 nm, over time as a proxy for GPX4 activity. The rationale for this is that as GPX4 reduces phospholipid hydroperoxides, it oxidizes glutathione, which is then regenerated by glutathione reductase at the expense of NADPH. NADPH is added to the assay in excess such that the reduction of oxidized glutathione is not the rate-limiting reaction in the assay. Thus, the resulting reduction in NADPH absorbance over time reflects GPX4 activity.

The GPX4-specific substrate phosphatidylcholine hydroperoxide (PCOOH) was prepared by enzymatic hydroperoxidation. Specifically, 0.3 mM phosphatidylcholines (Sigma-Aldrich) were incubated with 0.7 mg soybean lipoxidase type IV (Sigma-Aldrich) in 22 mL of 0.2 M Tris-HCl pH 8.8 with 3 mM sodium deoxycholate at room temperature under continuous stirring for 30 minutes. A Sep-Pak C18 cartridge (Waters-Millipore) was pre-washed with methanol and equilibrated with water, then the substrate mixture was loaded. The column was washed with 10 volumes of water and PCOOH was eluted with 2 mL of methanol.

To prepare the cell lysate containing native GPX4, A-673 cells were cultured with 20 nM sodium selenite to confluency over two days then harvested and washed with cold PBS. Cells were then resuspended at 10 million cells per 1 mL LCW lysis buffer (0.5% peroxide-free Triton X-100, 0.5% sodium deoxycholate salt, 150 mM NaCl, 20 mM Tris-HCl pH 7.5, 10 mM EDTA, 30 mM sodium pyrophosphate, and complete protease inhibitor cocktail), vortexed for 30 seconds, and incubated on ice for 15 minutes. After lysis, the lysates were centrifuged at 14,000×g and 4° C. for 15 minutes. The supernatants were transferred to new pre-chilled tube and the debris pellet was discarded. The protein concentration in the lysate was determined for normalization using the BCA Protein Assay Kit (Pierce).

To measure GPX4 activity, 50 μL of cell lysate was combined with 200 μL activity assay buffer (0.1% Triton X-100, 100 mM Tris-HCl pH 8.0, 10 mM sodium azide, 5 mM EDTA, 0.6 IU/mL glutathione reductase, 0.5 mM NADPH and 3 mM GSH) and compounds of interest in a clear 96-well plate and incubated at 37° C. for 45 minutes. Compounds were dissolved in DMSO. All conditions were tested in triplicate. PCOOH substrate was added to the mixture to initiate GPX4 activity just before reading the plate. The absorbance of NADPH at 340 nm was monitored kinetically over time for 25 minutes. Buffer-only controls and controls without substrate containing only the methanol vehicle were performed to assess background and baseline signal. GPX4 activity was calculated as previously reported by Stolwijk et al. 2020 and results were analyzed in GraphPad Prism 9.

### GPX1 Activity Assay in Cell Lysates

Determination of GPX1 activity in cell lysates was performed as previously described by Stolwijk et al. with some modifications (Stolwijk et al. 2020). GPX1 and GPX4 activity were not evaluated simultaneously in the same assay as in the published protocol to maximize signal-to-noise with the GPX1 substrate. Again, NADPH consumption, as determined by the reduction in the absorbance at 340 nm, over time was monitored as a proxy for GPX1 activity, which, like GPX4, reduces its substrates using glutathione as a reductant. 100 μM hydrogen peroxide was selected as the GPX1 substrate in this assay. A-673 cell lysates and assay setup were performed as above for the GPX4 activity assay. All conditions were tested in triplicate. Buffer-only controls and controls without substrate containing only Milli-Q water vehicle were performed to assess background and baseline signal. GPX1 activity was calculated as reported by Stolwijk et al. 2020 and results were analyzed in GraphPad Prism 9.

### C11-BODIPY Lipid Peroxidation Flow Cytometry Assay

Lipid peroxidation was assessed by flow cytometry using BODIPY™ 581/591 C11 (C11-BODIPY) (Invitrogen) following a previously published protocol with minor modifications (Martinez et al. 2020). HT-1080 cells were seeded at 250,000 cells per well in a 6-well plate and incubated overnight at 37° C. and 5% CO₂. The next day, cells were treated with compounds of interest with and without 10 μM Fer-1 for 2 hours in the incubator. 150 nM RSL3 with and without Fer-1 were included as positive controls. Compounds were dissolved in DMSO. A vehicle only control was performed for comparison to baseline. After 2 hours, cells were stained with 1.5 μM C11-BODIPY for 20 minutes. Cells were harvested with trypsin, then washed and resuspended in 500 μL HBSS. The cell suspension was filtered through nylon mesh (35 μm, cell strainer) to remove cell aggregates then run on a CytoFLEX flow cytometer (Beckman Coulter). 10,000 events were recorded per sample on the FL1 channel with gating to record live single cells only (gate constructed from the vehicle control). Data were analyzed in FlowJo 11.

### Microsomal Stability Assays

Human and mouse microsomal stability assays were performed by Curia using the following conditions: 1 μM test compound, 1 mg/mL microsomal protein, 800 μL incubation volume, quenched with 0.5% acetonitrile, pH 7.4, incubation at 37° C. Microsomal protein concentration for testosterone was 0.25 mg/ml for mouse. The incubation timepoints measured were 0, 5, 10, 15, 30, and 45 minutes. All samples were prepared in duplicate and analyzed by LC-MS/MS.

### Aqueous Solubility

Kinetic aqueous solubility assays were performed by Curia at ambient temperature with 1% DMSO in 0.1 M Phosphate Buffered Saline pH 7.4. After a 1-hour incubation, samples were analyzed by UV/Vis absorbance.

### Drug-Tolerant Persister Cell Model

Compounds were tested in a drug-tolerant persister (DTP) cell model of minimal residual disease derived from PC9 non-small cell lung carcinoma cells. PC9 cells are treated for 6 days with 15 μM erlotinib. At that point, the remaining population are termed DTP cells and display the typical markers CD133 and CD24 on flow cytometry. For the assay, DTP cells were seeded at 50,000 cells per well in 24-well plates with 1 μM erlotinib and incubated overnight at 37° C. and 5% CO₂. At the same time, parental cells are seeded at the same density. The next day, cells are treated with the appropriate drug treatments, including 1 μM RSL3, 10 μM erlotinib, or the indicated concentrations of the compounds of interest. DMSO only controls and blank wells (no cells) are also included for later signal normalization during data analysis. All test conditions are performed in technical quadruplicate. Cell viability is assessed 48-hours after treatment using CellTiter-Glo (Promega) according to manufacturer's instructions and luminescence is measured on a Victor X5 plate reader. Statistical significance was determined using two-way ANOVA with Sidak multiple hypothesis correction; *p<0.01, ***p<0.001, ****p<0.0001.

### Example 2

Improved LOC1886 Analogs of Fragments and Leads that Bind to GPX4

Glutathione peroxidase 4 (GPX4) has been reported to be a promising therapeutic target for metastatic and drug-resistant cancers, based on the elevated dependency of the cancer cells on GPX4 lipid peroxide repair pathway during epithelial-mesenchymal transition (EMT) and the transformation into therapy-tolerant persister states. We proposed to exploit this finding to discover drug-like GPX4 inhibitors with favorable ADMET properties that can be developed as a first in class GPX4 inhibitor for drug-resistant cancers having a high EMT gene expression signature.

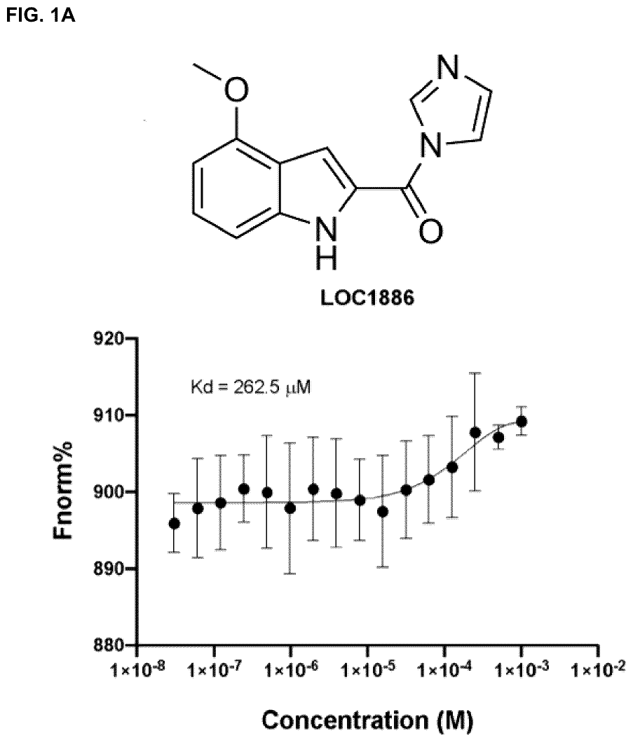

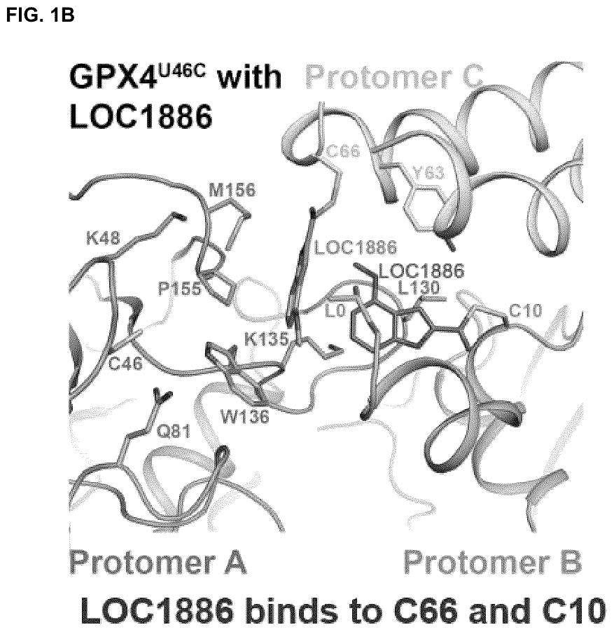

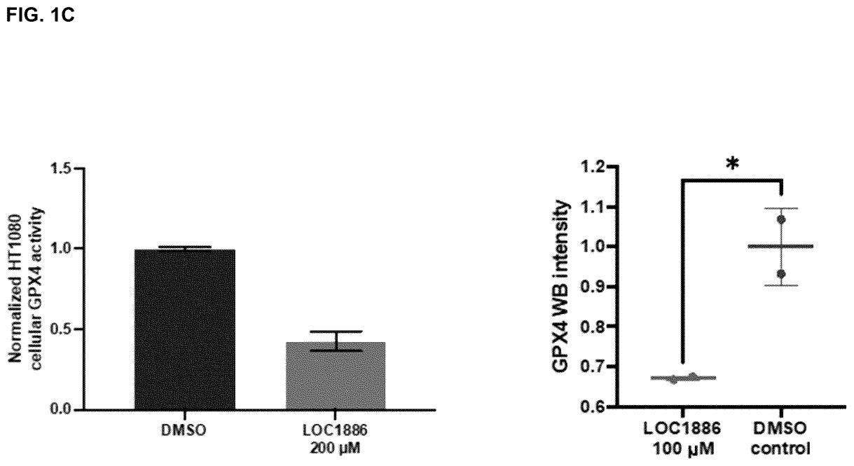

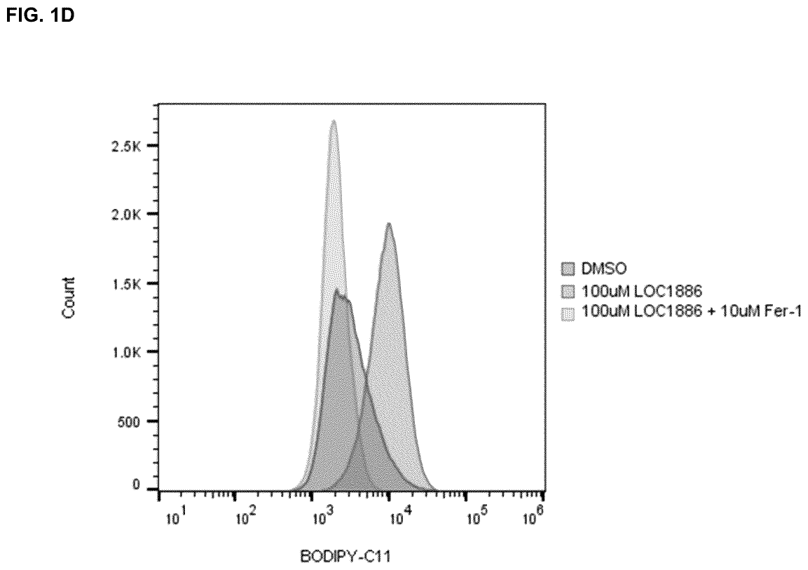

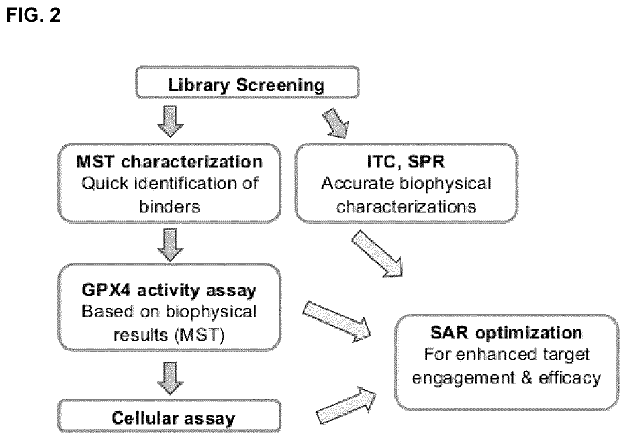

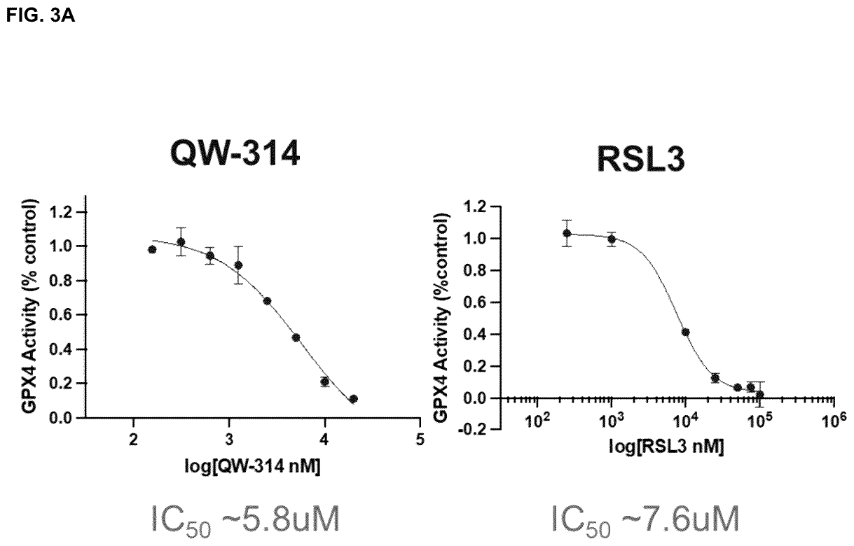

LOC1886 is GPX4 allosteric inhibitors identified from the initial biophysical screening of 10,095 Lead Optimized Compounds (LOC). LOC1886 covalently modifies GPX4 Cys66 (allosteric site 1) and Cys10 with a K_{d} of 262.5 μM (FIGS. 1A-1B). LOC1886 inhibits and degrades GPX4 (FIG. 1C) and induces lipid peroxidation in cells (FIG. 1D). By employing the established assay funnel designed for quick identification of potential lead compounds (FIG. 2), the SAR optimizations on LOC1886 were conducted and provided herein.

### SAR of LOC1886

For SAR optimizations based on LOC1886 scaffold, using the available crystal structures as guidance, numbers of LOC1886 analogs bearing a similar warhead as RSL3 were designed and synthesized (Scheme 1). Specifically, the imidazole warhead was replaced from the original LOC1886 with the chloroacetyl group from RSL3 for enhanced reactivity. The indole scaffold was substituted with an indazole and connected to the warhead via a 2-methylpropyl linker that extends further into the long hydrophobic cavity, bringing the indazole ring closer to W136 site and allowing for improved π-π stacking.

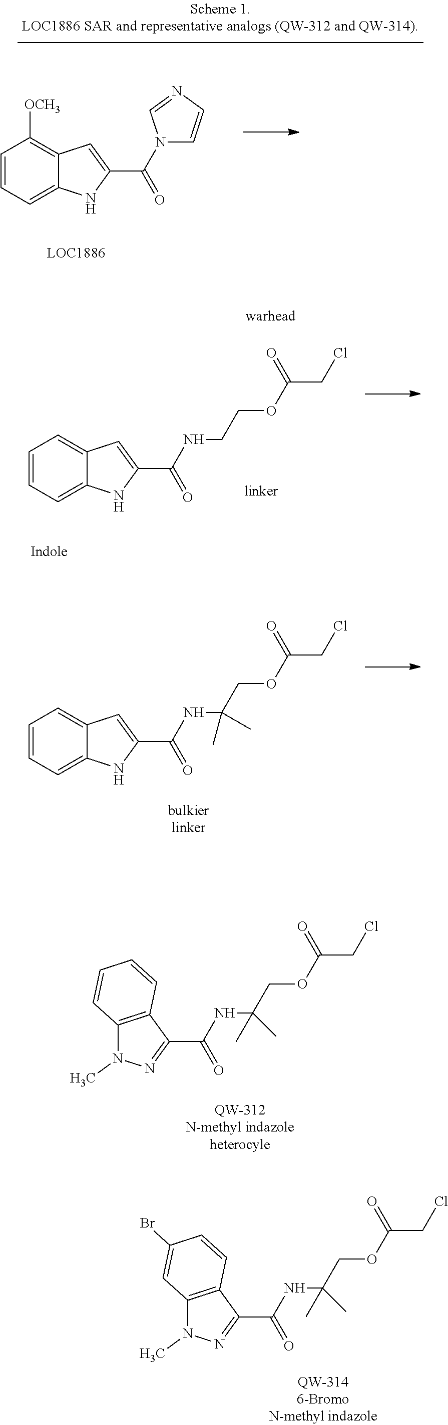 [SMILES: COc1cccc2[nH]c(C(=O)n3ccnc3)cc12 ; O=C(CCl)OCCNC(=O)c1cc2ccccc2[nH]1 ; CC(C)(COC(=O)CCl)NC(=O)c1cc2ccccc2[nH]1 ; Cn1nc(C(=O)NC(C)(C)COC(=O)CCl)c2ccccc21 ; Cn1nc(C(=O)NC(C)(C)COC(=O)CCl)c2ccc(Br)cc21]

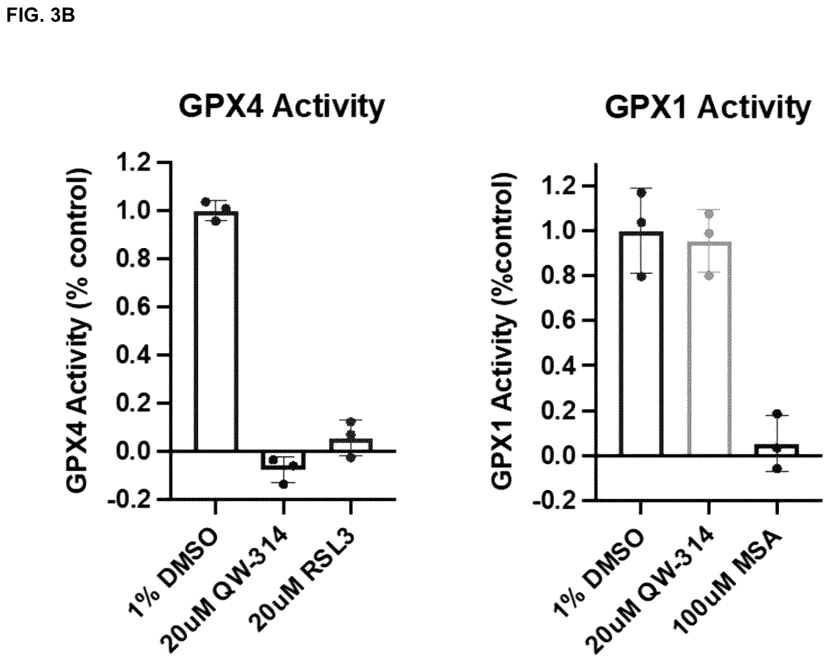

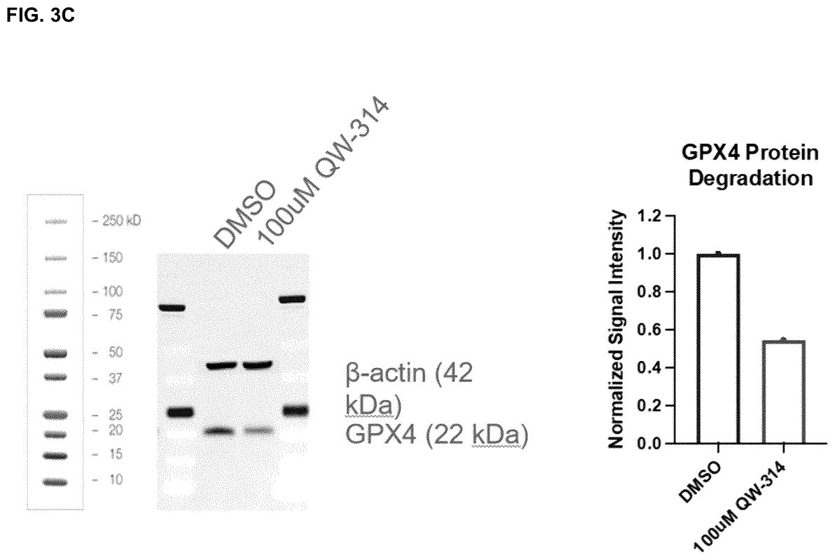

One of the LOC1886 analogs, QW-314, was found binding to GPX4 with a K_{d} of 5.8 μM, which is comparable to that of RSL3 and significantly improved from LOC1886 (FIG. 3A). QW-314 showed high selectivity for GPX4 over GPX1 (FIG. 3B) and induces GPX4 protein degradation (FIG. 3C).

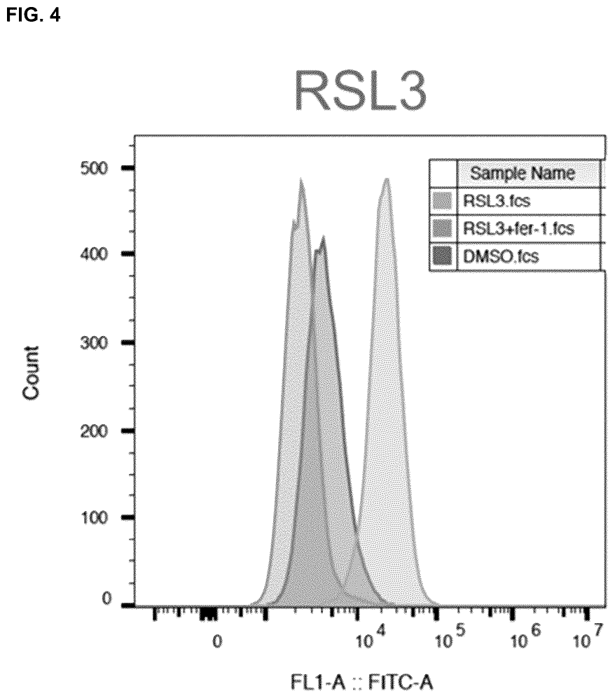

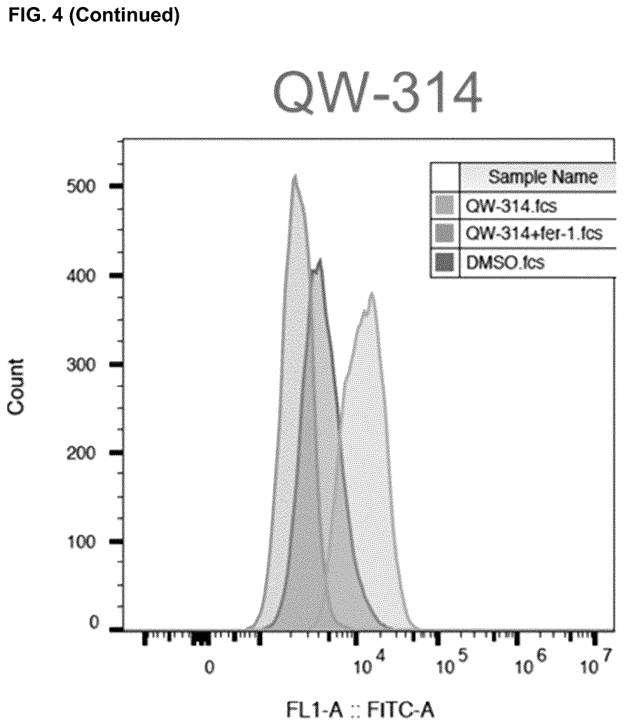

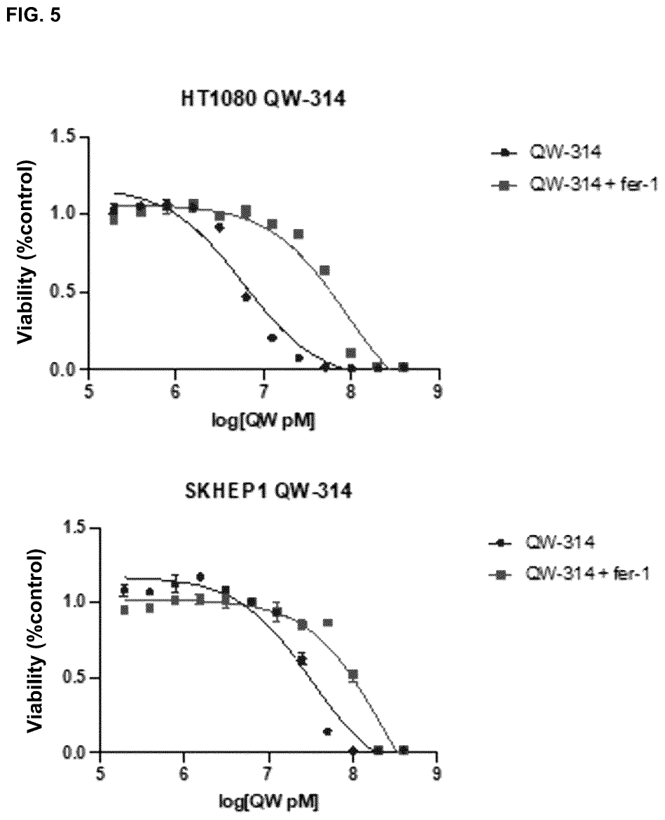

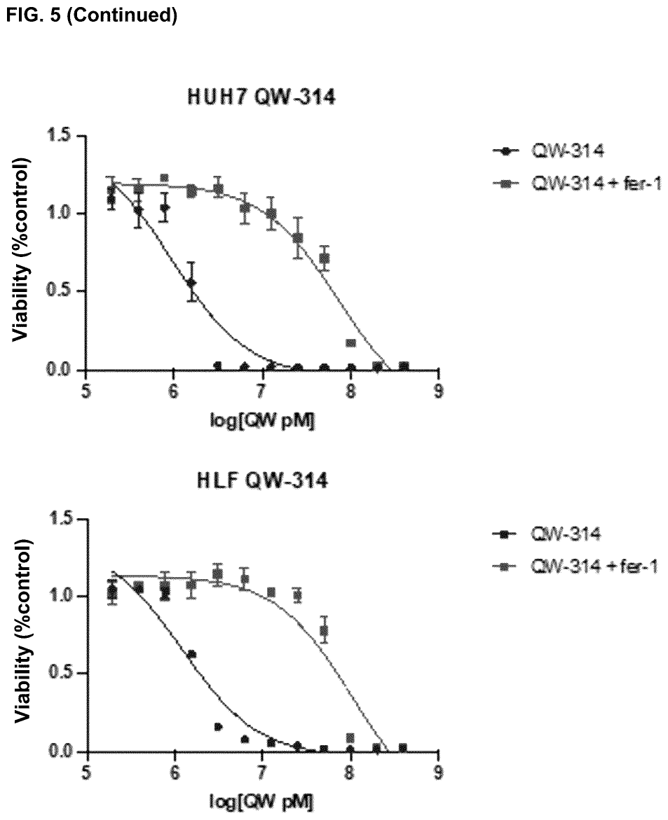

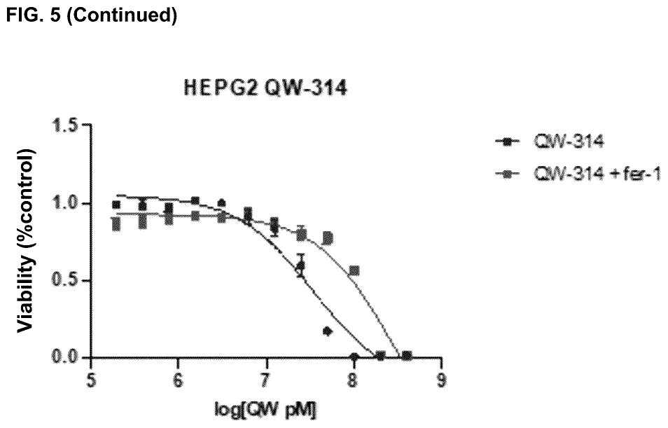

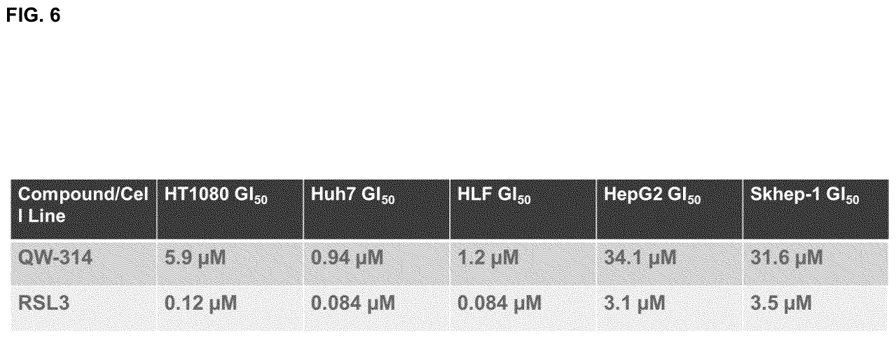

QW-314 has been tested for induction of lipid peroxidation using flow cytometry with C11-BODIPY in HT1080 fibrosarcoma cells, an established ferroptosis model system (FIG. 4). 2.5×10⁵ HT1080 cells per well were seeded in 6-well plates and cells were treated with drug the next day for 2 hours, then stained with 1.5 uM C11-BODIPY for 20 min. Cells were then washed with HBSS and harvested for analysis. QW-314 showed increased lipid peroxidation that is rescuable by ferrostatin-1 (fer-1), a ferroptosis inhibitor, potentially suggesting GPX4 inhibition in cells, and induced ferrostatin-1 rescuable cell death in HT1080, Huh7, HLF, SKHEP-1 and HEPG2 liver cancer cell lines (FIG. 5). GI₅₀ values in two HCC cell lines (Huh7 and HLF) show that QW-314 has ˜1 μM micromolar potency, which is approximately 10 times less potent than RSL3 in cells (FIG. 6). Further optimization of these compounds may be required to improve potency and specificity in the cellular context.

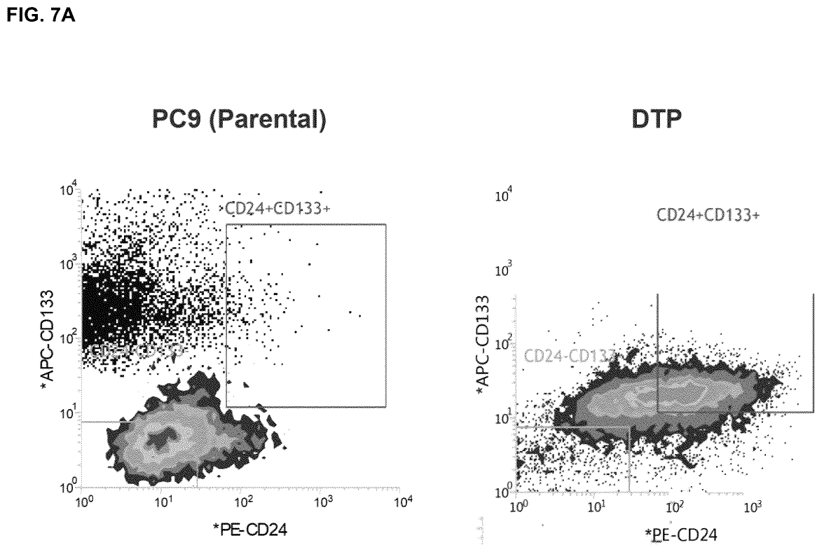

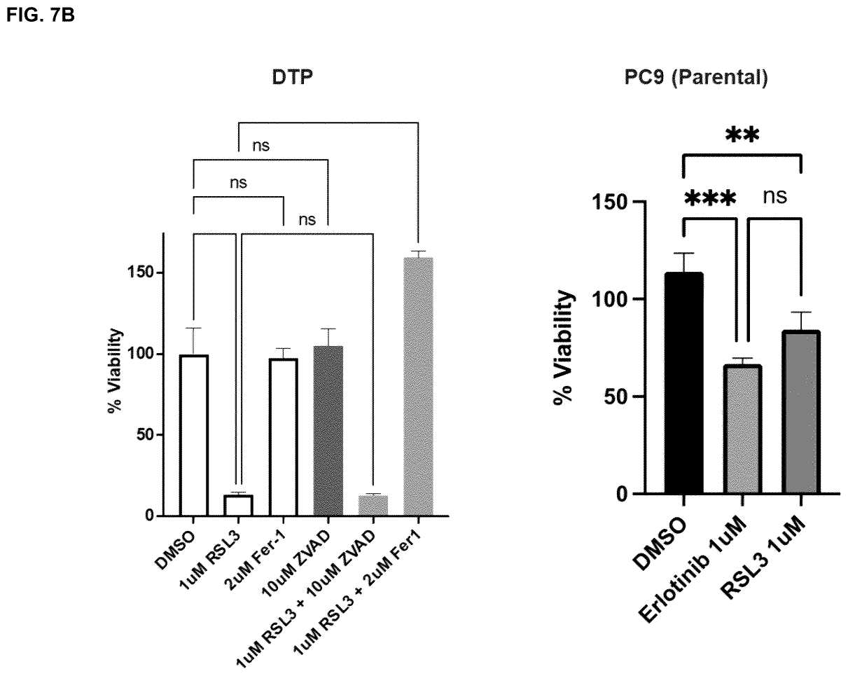

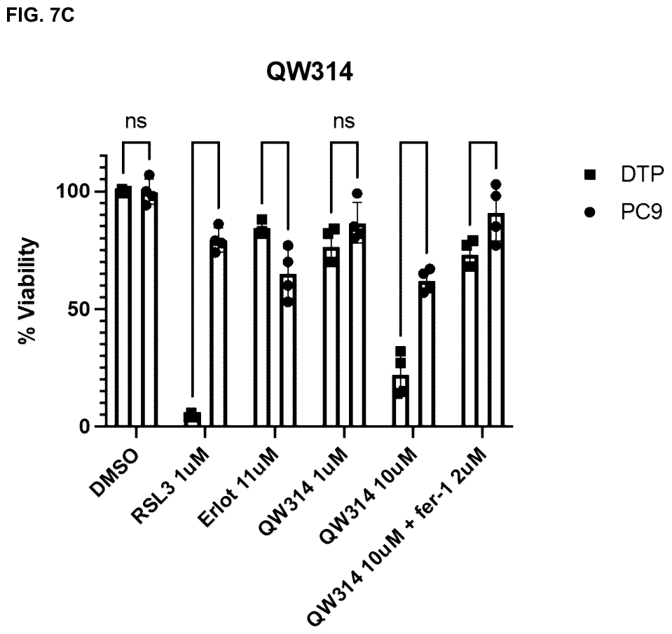

In addition, QW-314 has been further tested using a newly developed non small cell lung carcinoma (NSCLC) model of drug tolerant persister (DTP) cells. DTPs are a subpopulation of cells implicated in post chemotherapy relapses (PC9 cells). DTPs are CD133 and C24 positive (FIG. 7A). They are specifically sensitive to GPX4 inhibitors (FIG. 7B). Targeting DTPs may open up previously inaccessible options for prevention of cancer recurrence. QW-314 showed selective lethality in DTPs vs PC9 parental cells in a dose dependent manner and was rescued by fer-1 (FIG. 7C).

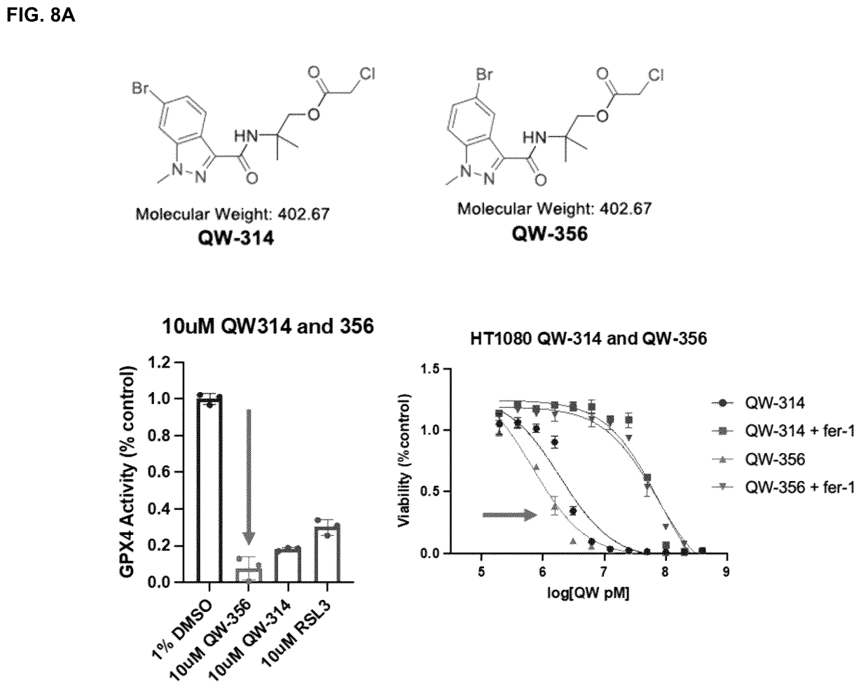

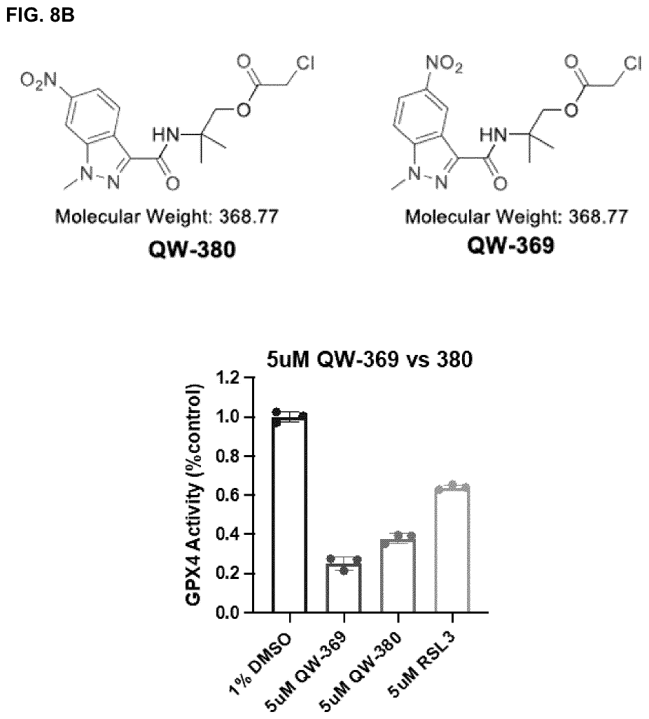

Further modifications based on QW-314 by changing the electron withdrawing group and/or its position showed improved potency in biochemical and cellular assays. The in vitro inhibitory efficiencies of those analogs on GPX4 were further validated using the NADPH-coupled biochemical assay. Among the analogs tested, QW-356 showed significant improvement on GPX4 inhibition over QW-314 and RSL3 (FIG. 8A), indicating a promising substitution at the 5 position. Similar effect was observed between QW-369 and QW-380 (FIG. 8B).

### Example 3

### Further Optimization Based on QW-314

For further inhibitor optimization, the indazole ring on the LOC1886 scaffold was also modified with diverse substitution patterns and electronic effects to modulate the stacking strength (Scheme 2). In some analogs, we chose to link the indazole to the 2-methylpropyl linker via an amide bond to enhance the solubility and prevent aggregation, while in some other analogs an acetate bond was used.

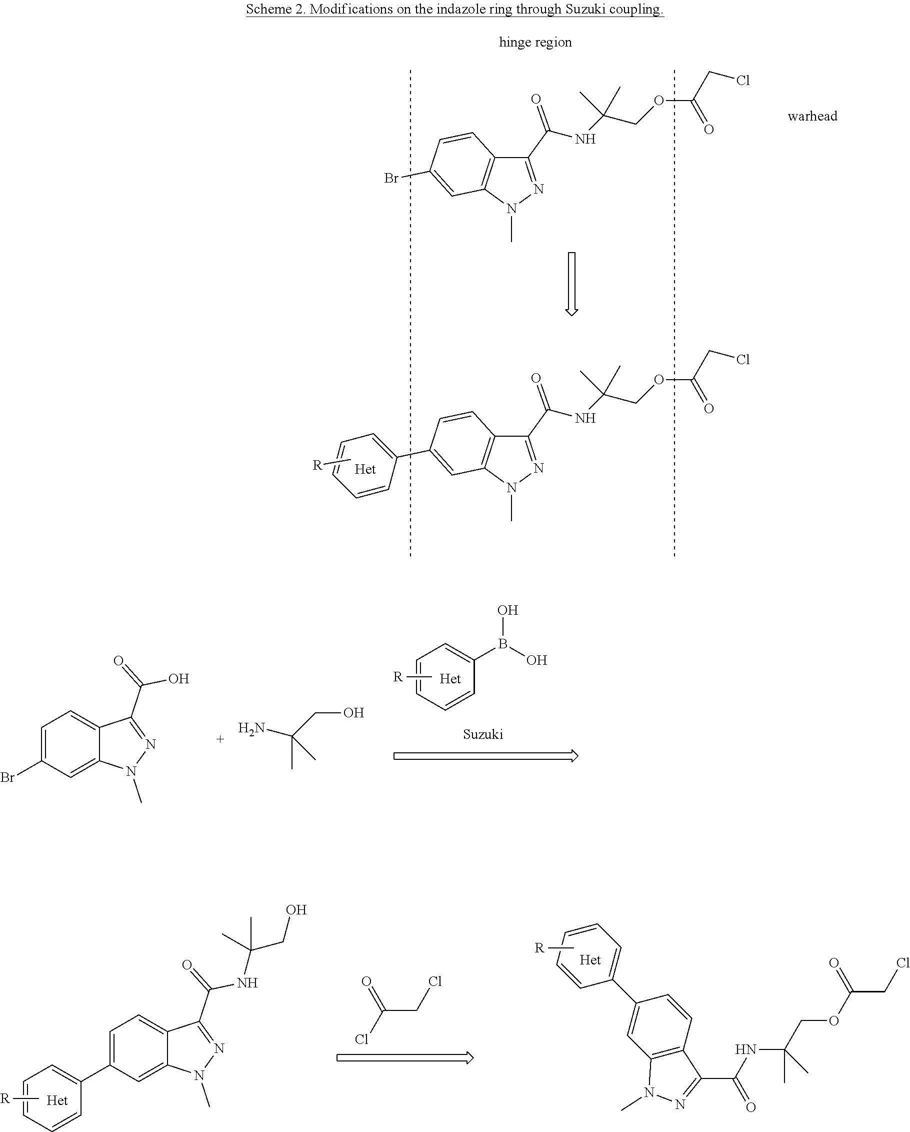

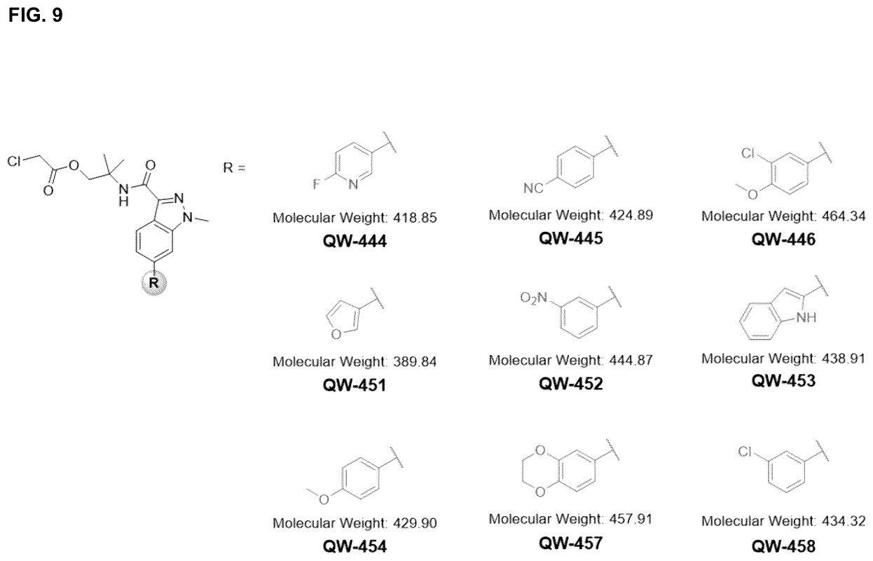

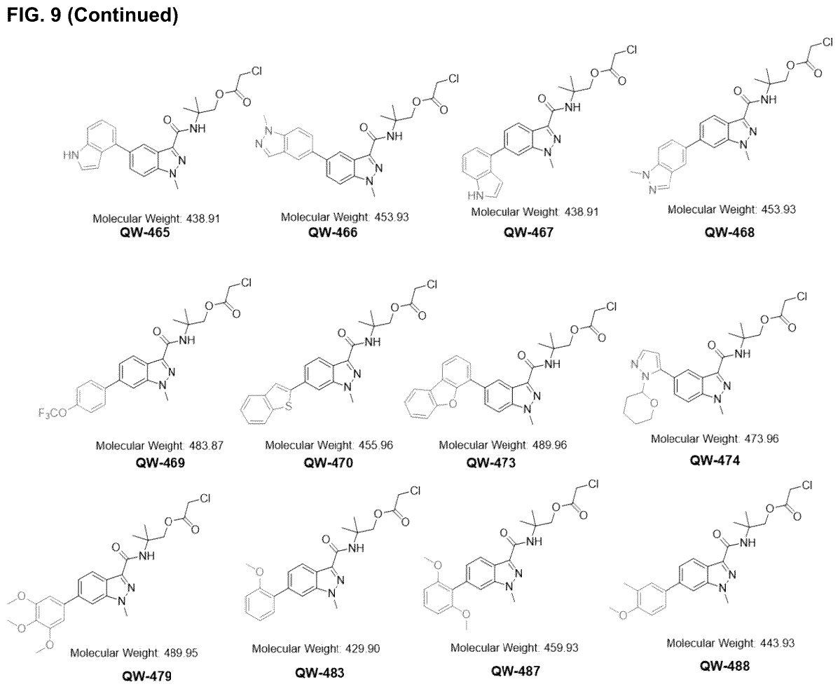

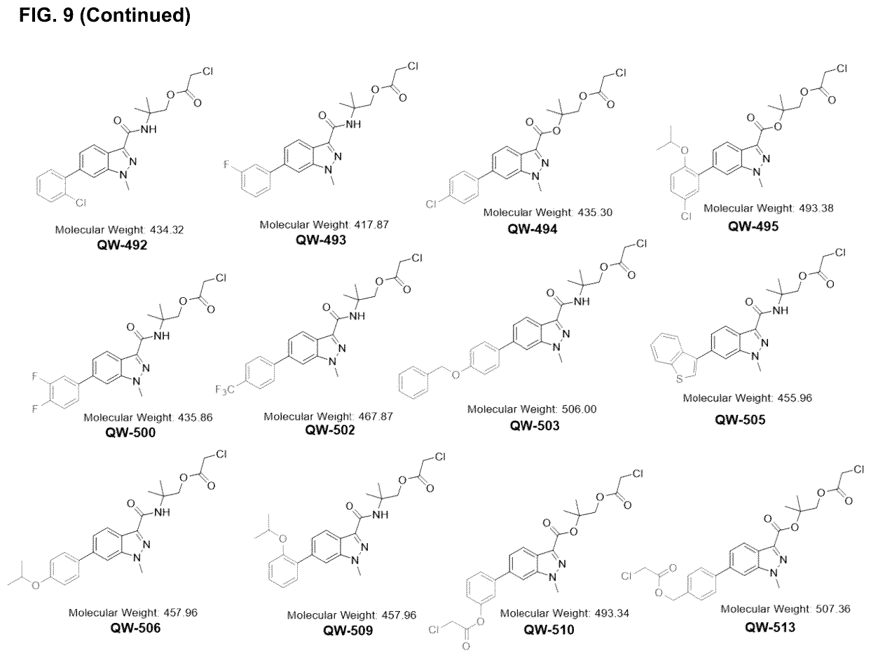

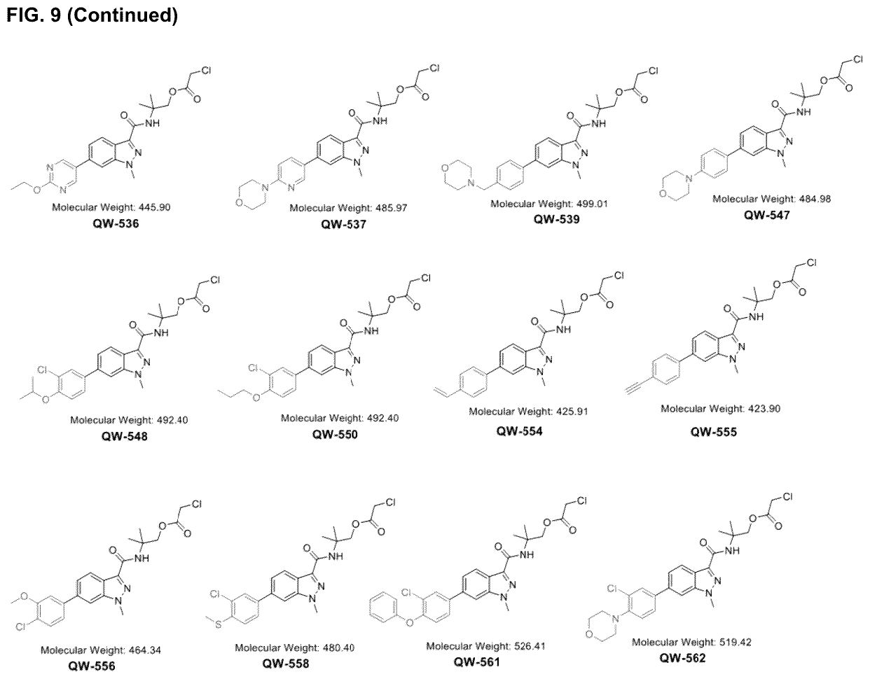

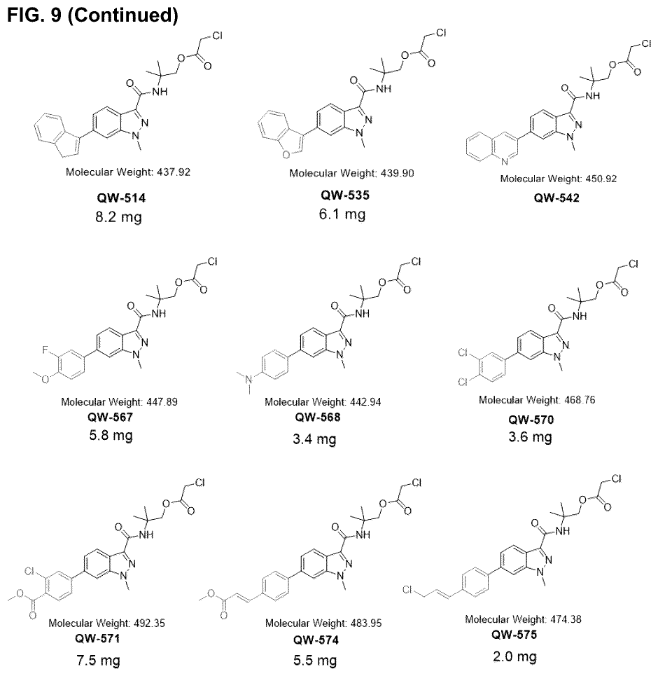

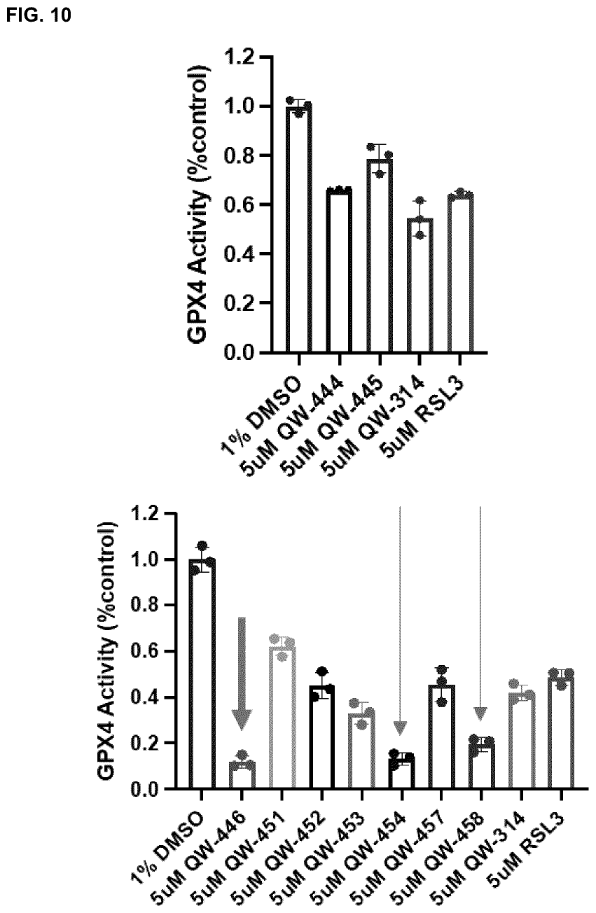

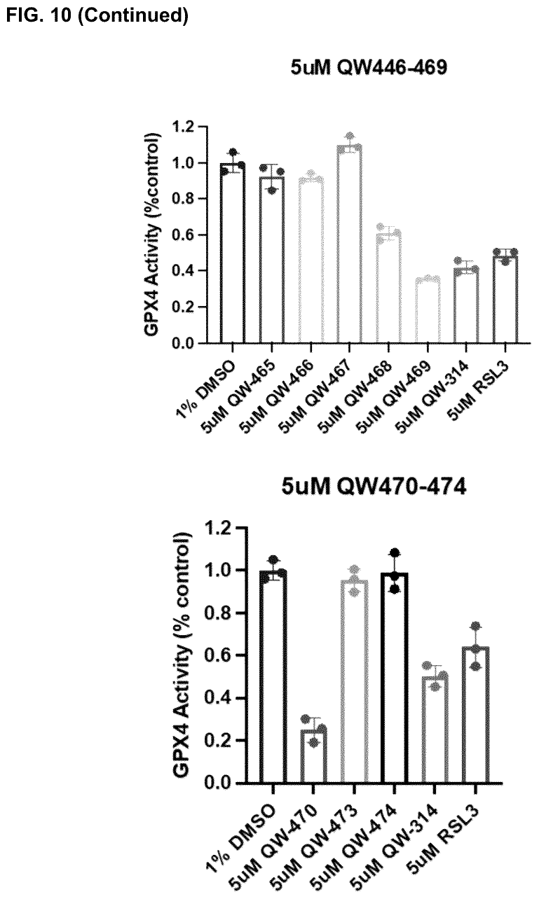

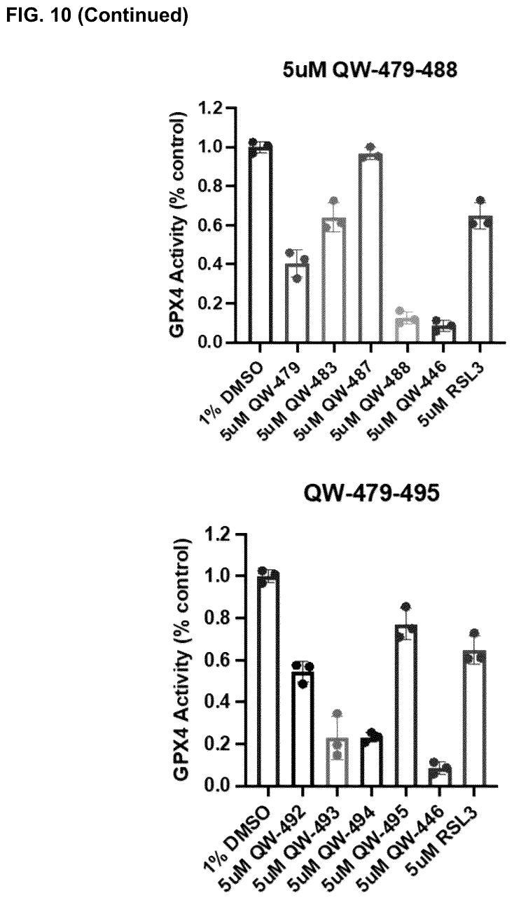

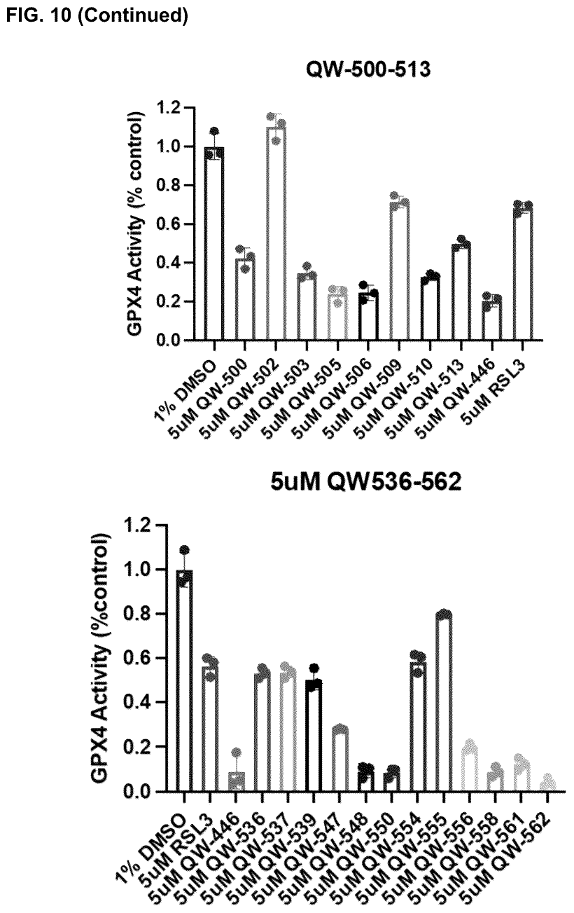

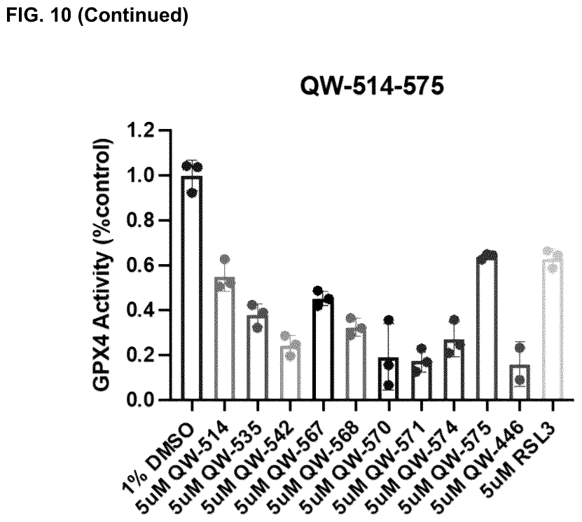

Newly synthesized analogs of QW-314 are shown in FIG. 9. These compounds have also been screened in the NADPH-coupled GPX4 activity assay (FIG. 10). Compounds with poor solubility in the assay buffer were assessed at lower concentrations. Solubility of some compounds were tested and listed in Table 1.

TABLE 1
Solubility of QW-314 Analogs.

| Compound ID | λ (nm) | Solubility (μM) |
|---|---|---|
| QW-446 | 260 | 22 |
| QW-454 | 270 | 2.9 |
| QW-458 | 260 | 6.1 |
| QW-488 | 270 | 5.4 |
| QW-505 | 310 | <1.3 |
| QW-506 | 270 | 1.7 |
| Verapamil | 280 | 88 |
| Tamoxifen | 280 | 4.9 |

Assay Conditions:
Percent Organic Solvent in Incubation: 1% DMSO
Incubation Time: 1 hour
Buffer System: Phosphate Buffered Saline pH 7.4
Temperature: Ambient
Positive Control: Verapamil and Tamoxifen
Analysis: UV/Vis Absorbance
Number of Replicates: 2

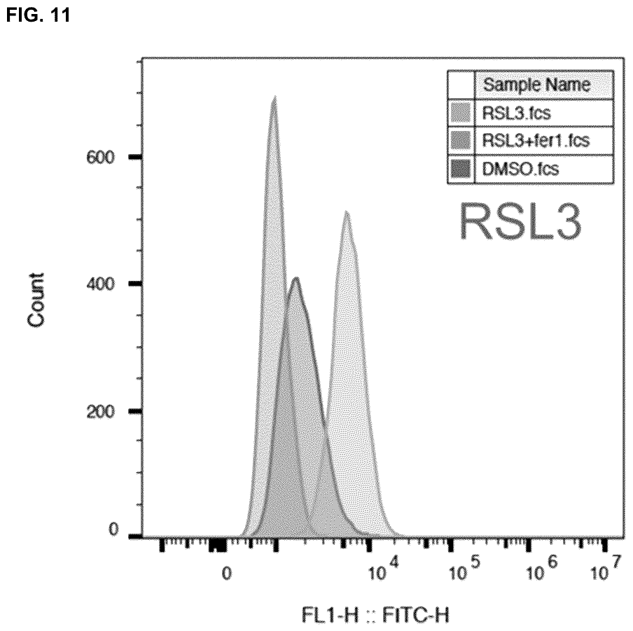

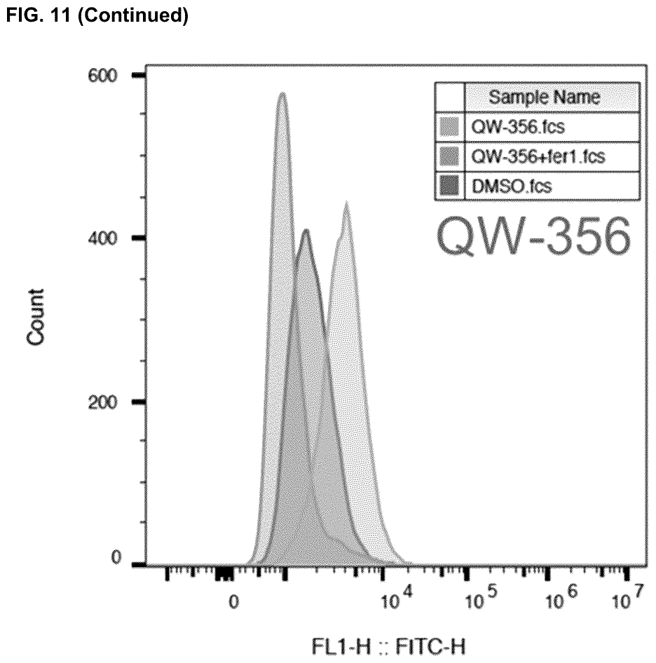

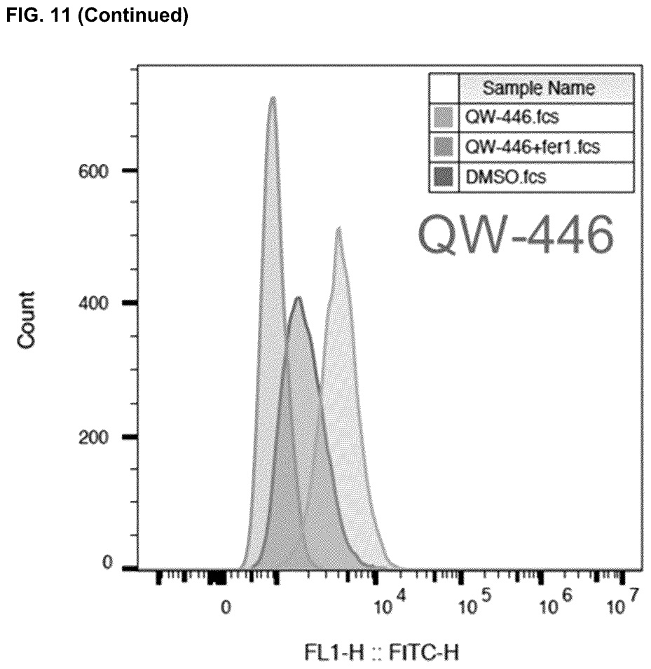

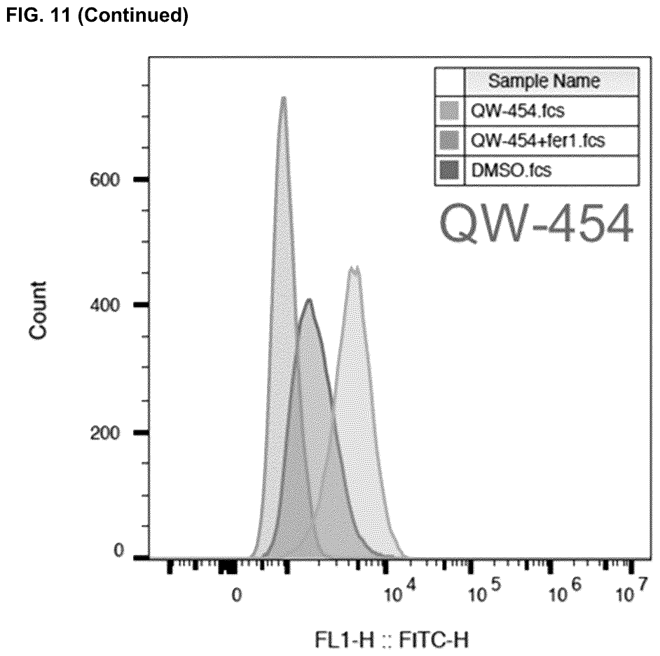

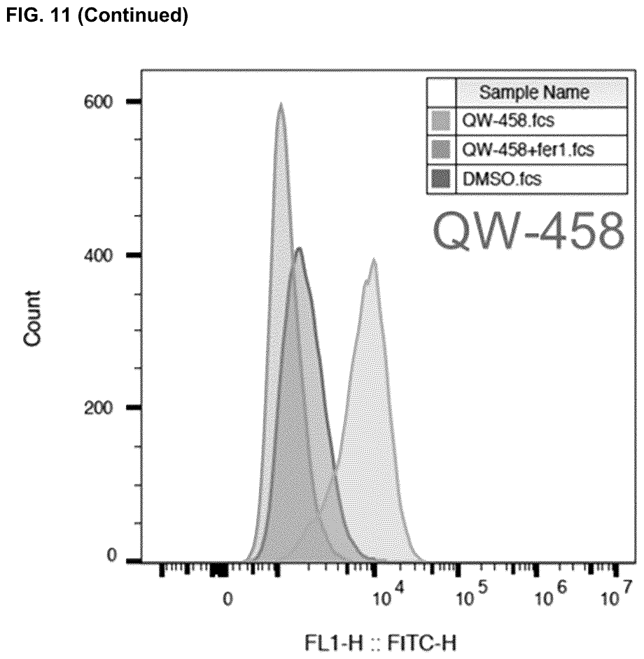

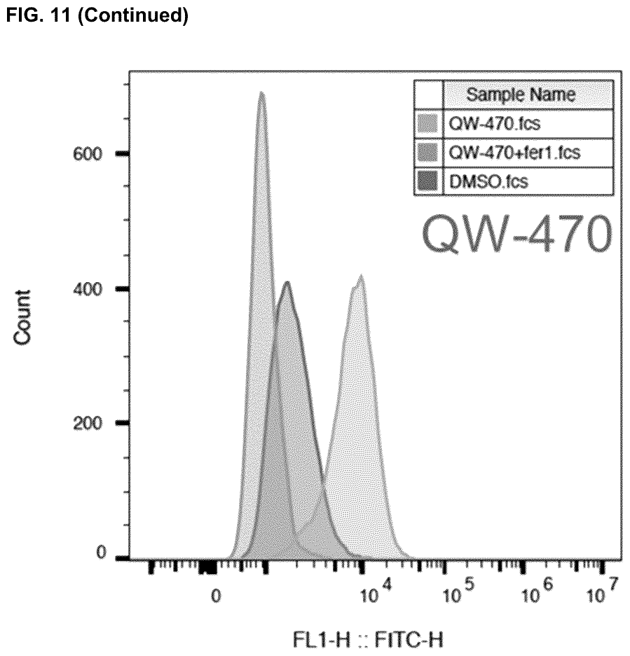

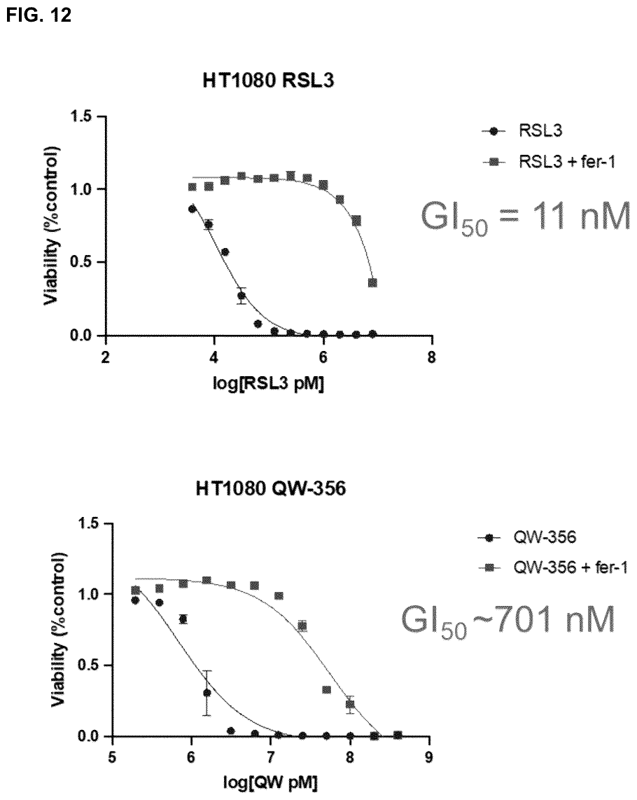

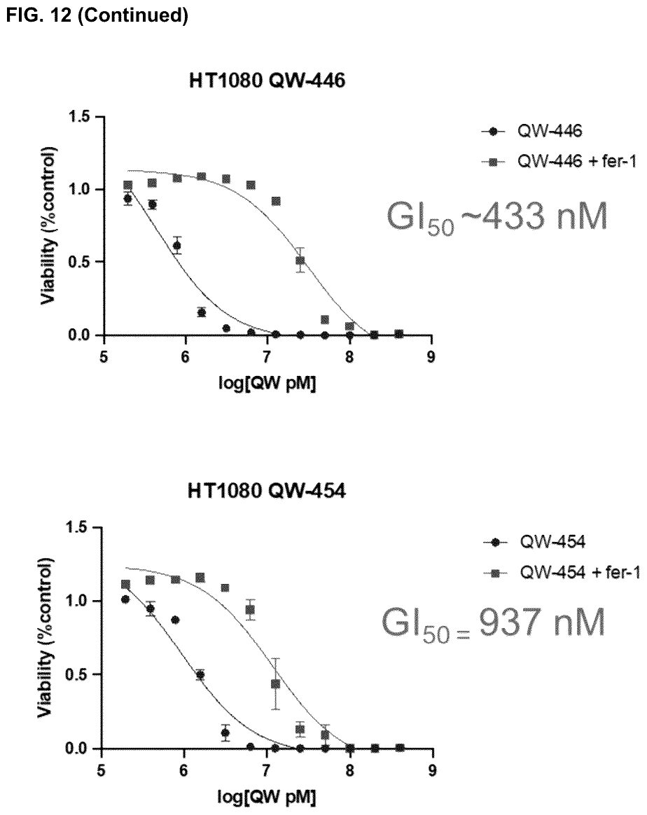

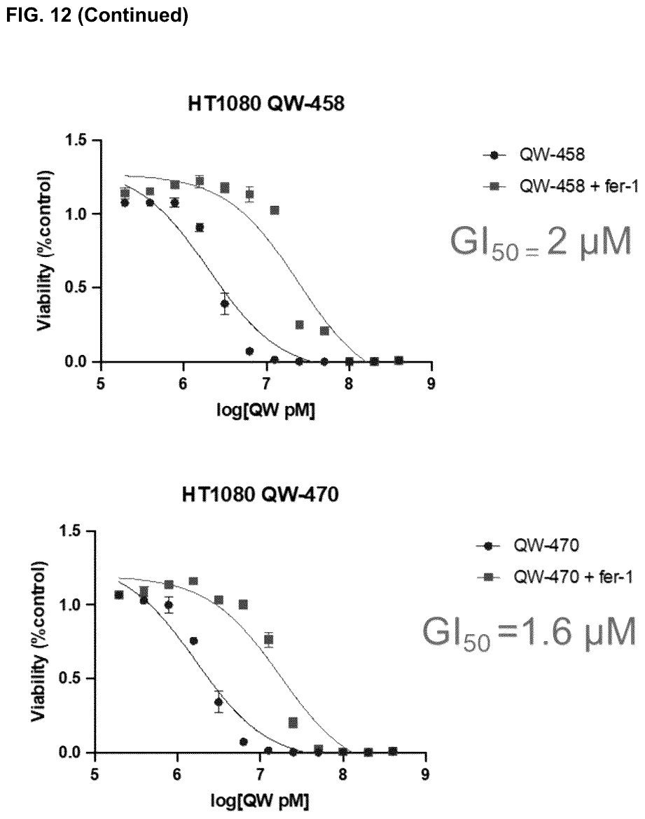

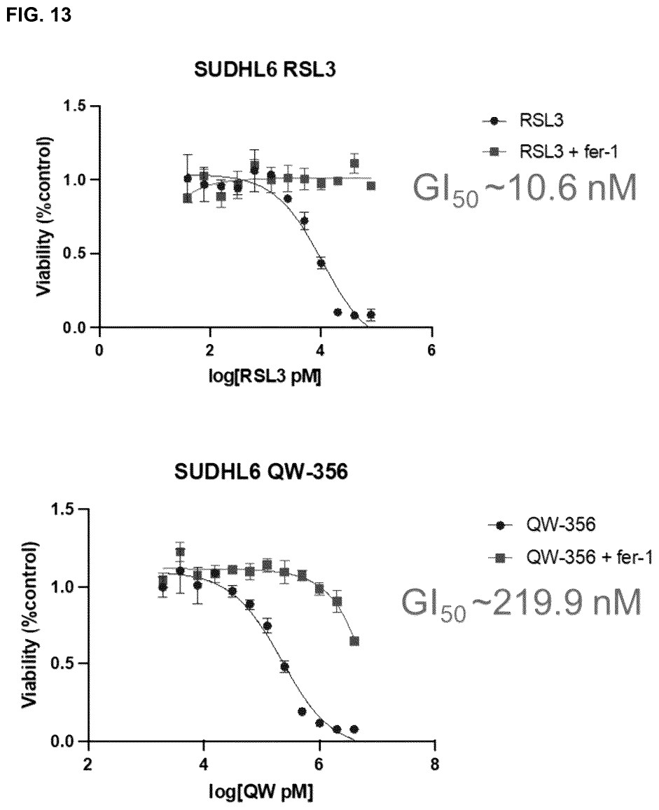

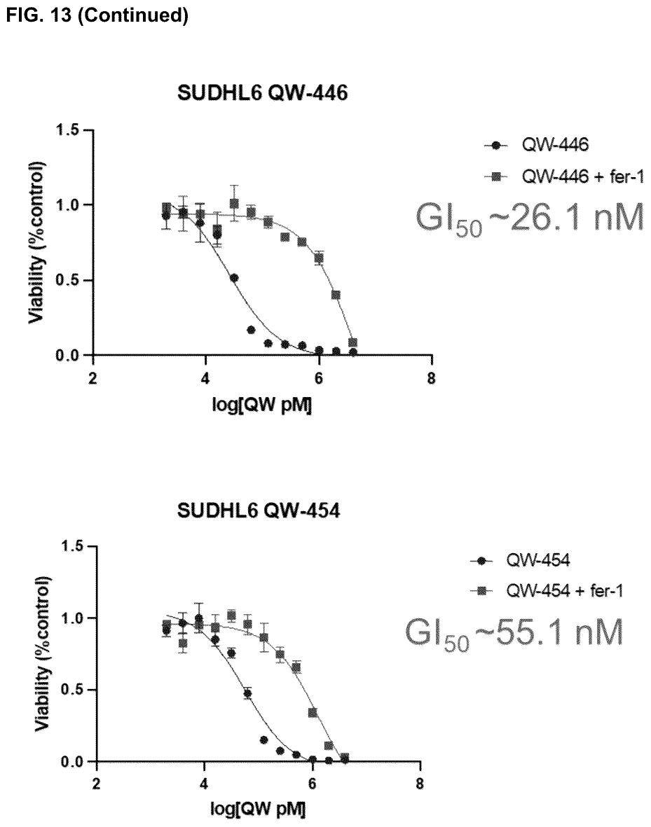

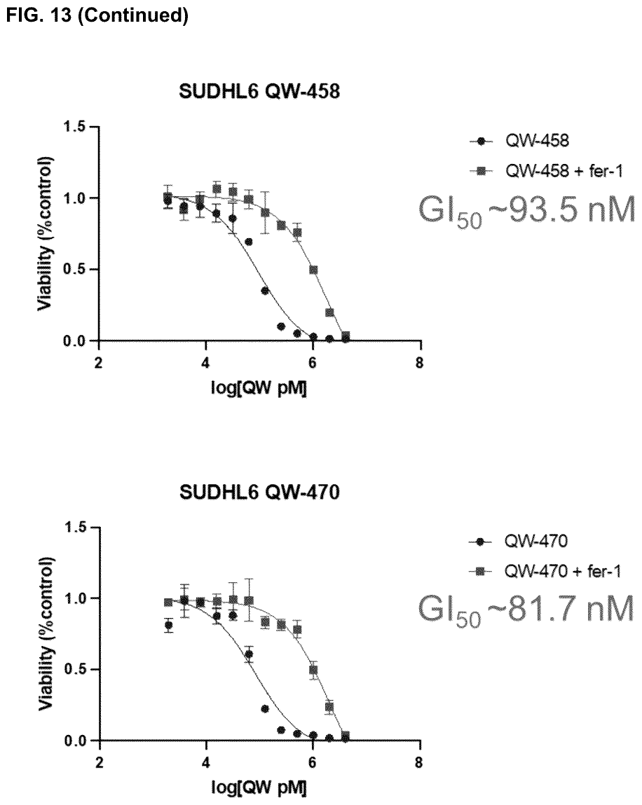

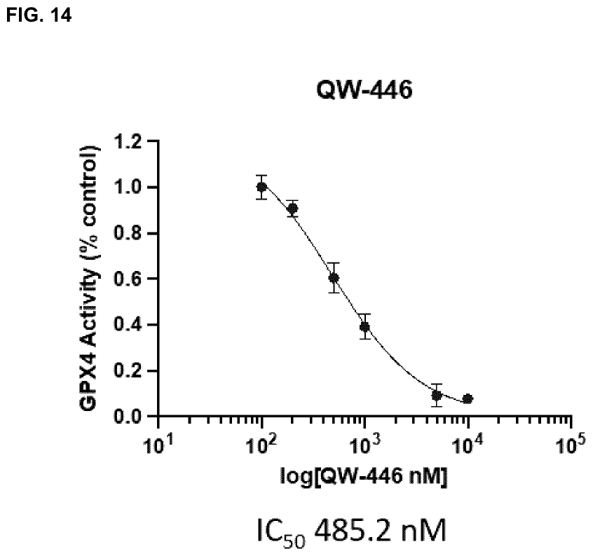

Many analogs show improved inhibition of GPX4 activity over QW-314, and a few compounds show greater than 50% inhibition of GPX4 activity (FIG. 10). In the cellular dose response assays, some of these QW-314 analogs showed fer-1 rescuable increase in lipid peroxidation (FIG. 11), and induced ferroptosis in moderately resistant HT1080 fibrosarcoma cells with sub-micromolar GI₅₀ values (FIG. 12). The same set of compounds induced ferroptosis in sensitive SUDHL6 DLBCL cells with even lower GI₅₀ values (FIG. 13), among which, QW-446 showed a potency that is similar to RSL3. The binding affinity of QW-446 to GPX4 was further measured by MST, resulting in a K_{d} of 485.2 nM, which is 15 times more potent than RSL3 (FIGS. 14 and 3A). Further characterization and validation of these compounds are currently in progress, and results will be used to inform further inhibitor optimization.

Taken together, these data suggested that we have identified numbers of analogs of LOC1886 with better binding affinity to GPX4, and their structures provided us with novel pharmacophores for further optimization.

### Example 4

### Additional Improved LOC1886 Analogs

As noted above, we identified several top leads by applying medicinal chemistry approaches to improve LOC1886, and found QW-446 as one of the best compounds. Nevertheless, metabolic and Plasma stability of QW-446 was not satisfying and required additional improvements. Further optimization of the LOC1886 scaffold was conducted to identify compounds with improved GPX4 inhibitory ability as well as improved drug-like properties.

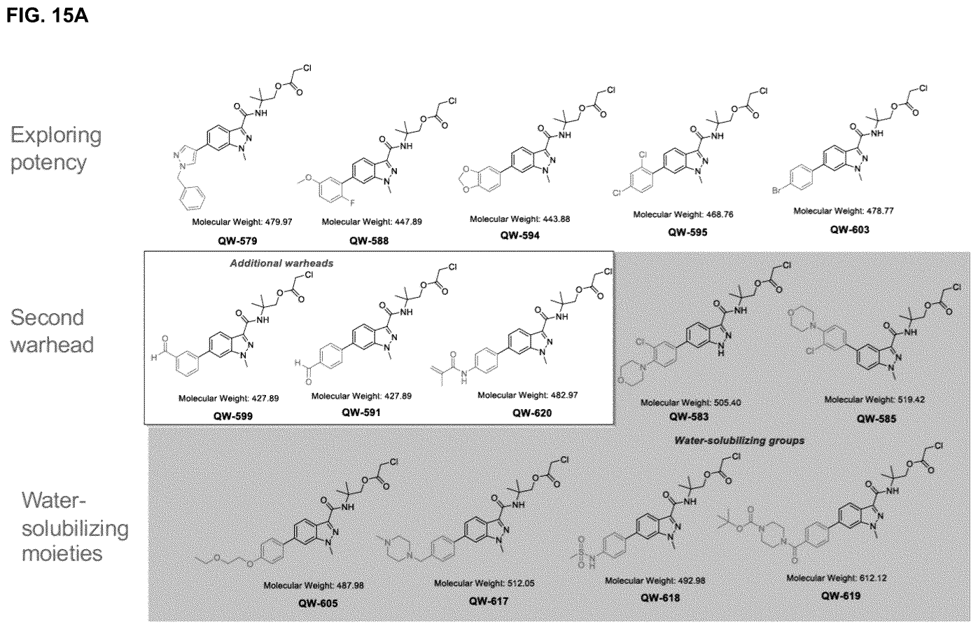

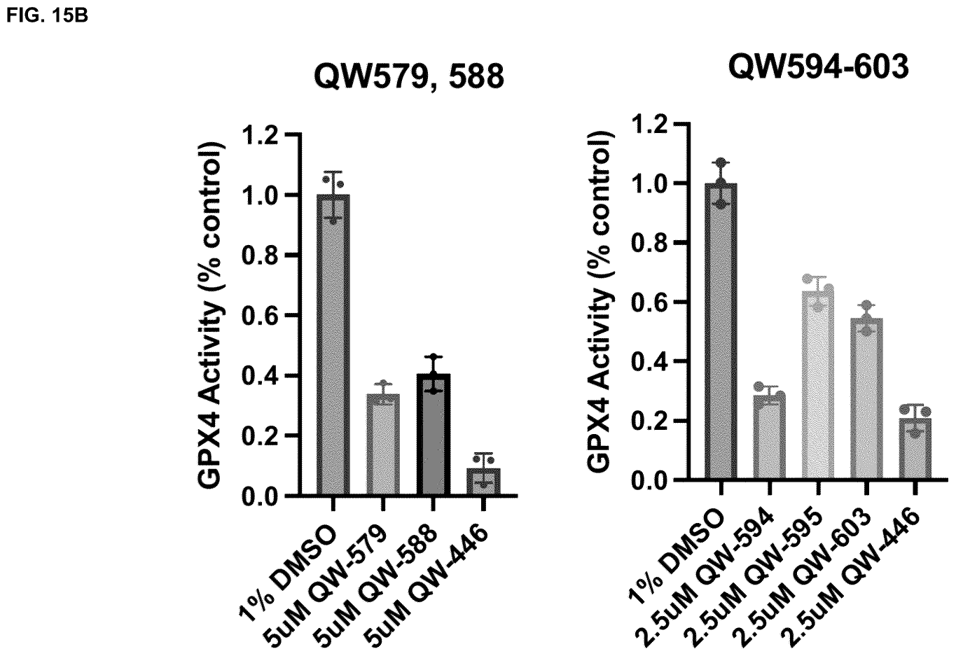

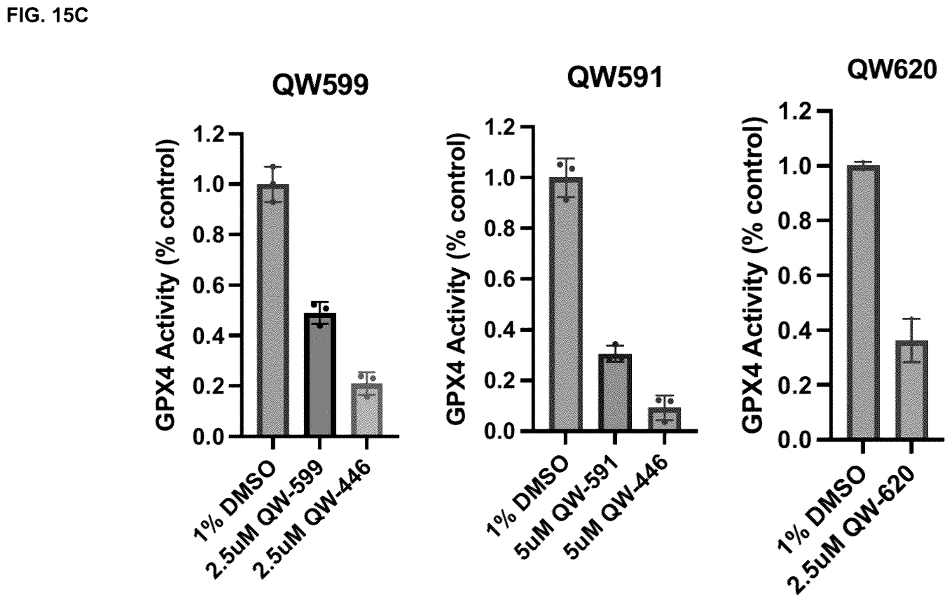

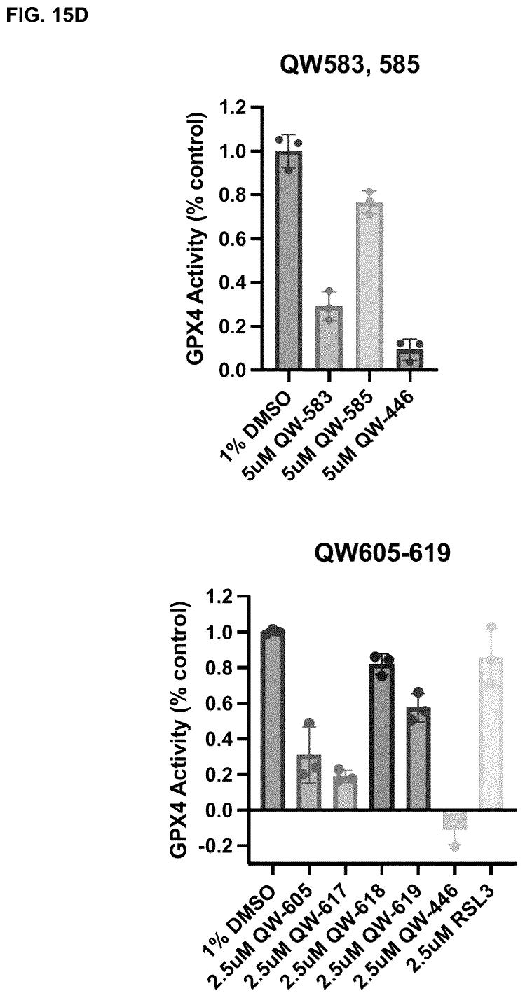

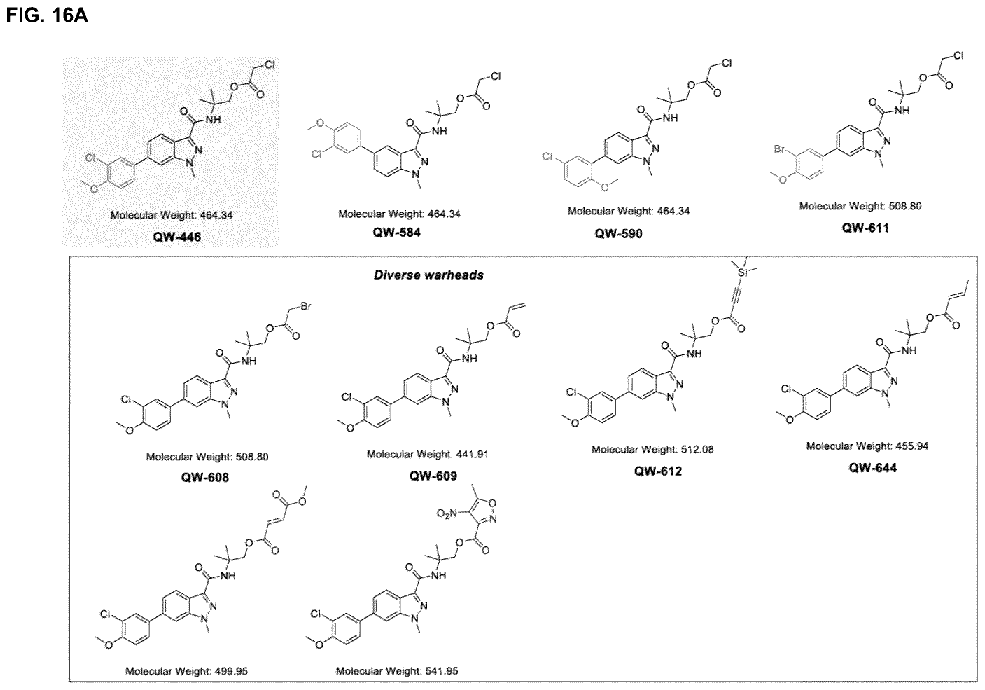

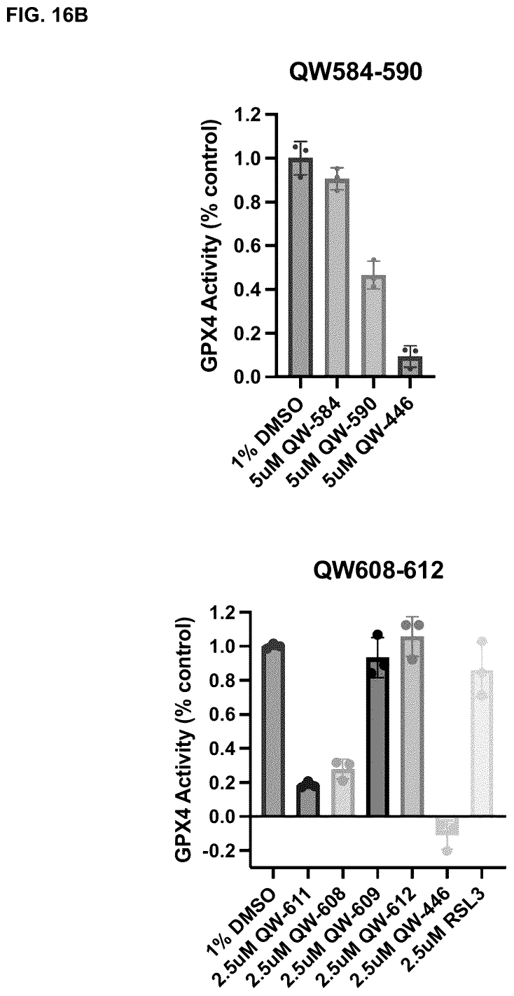

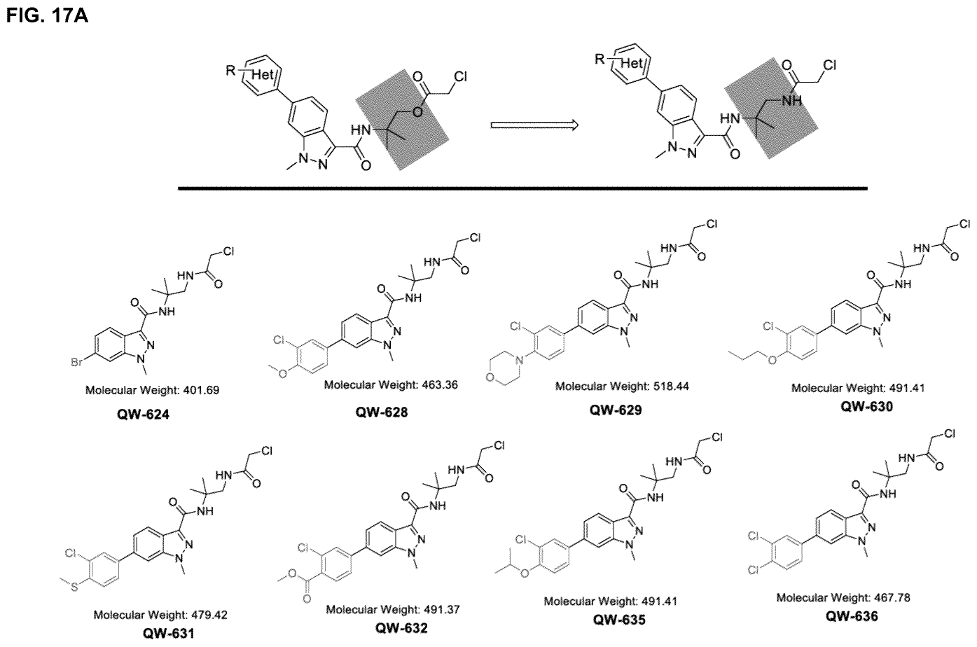

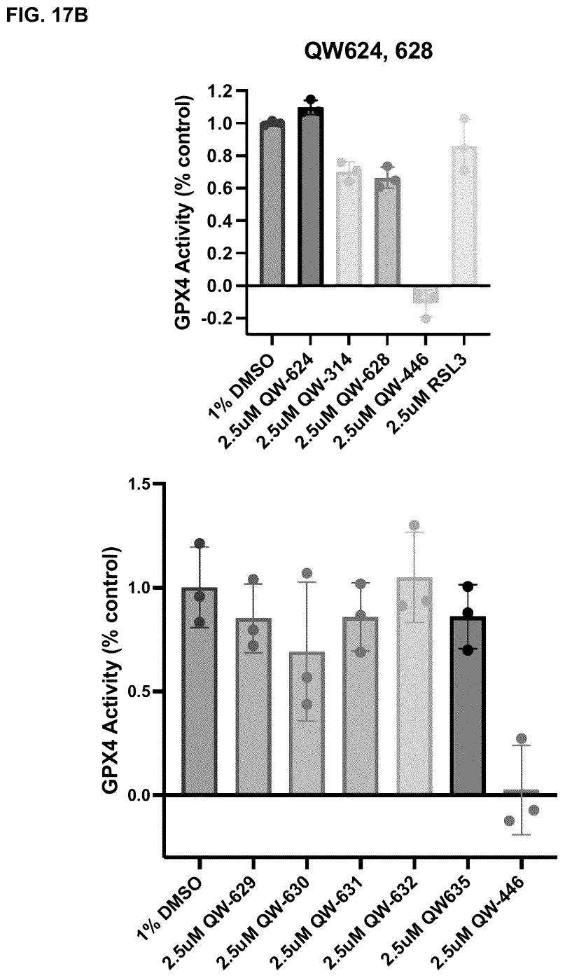

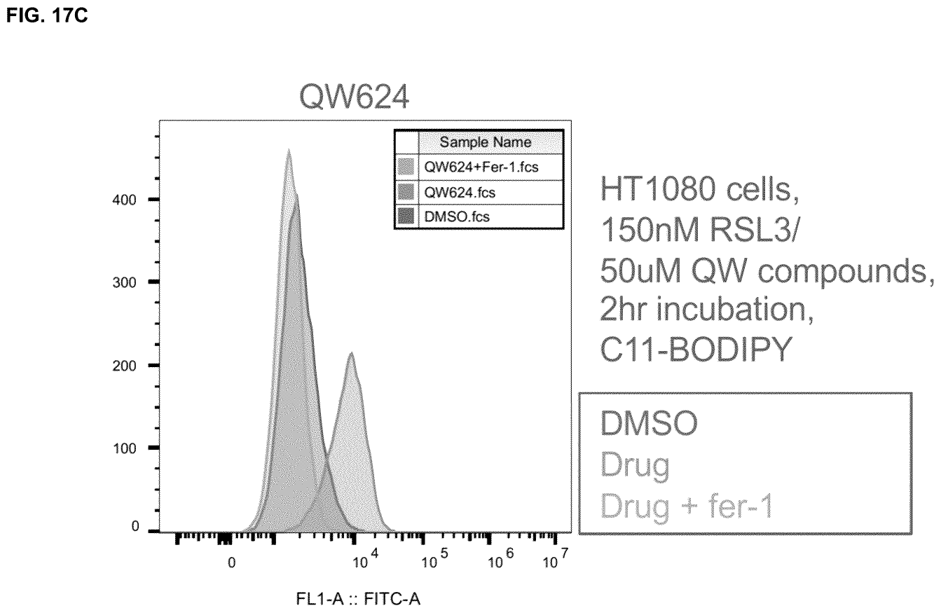

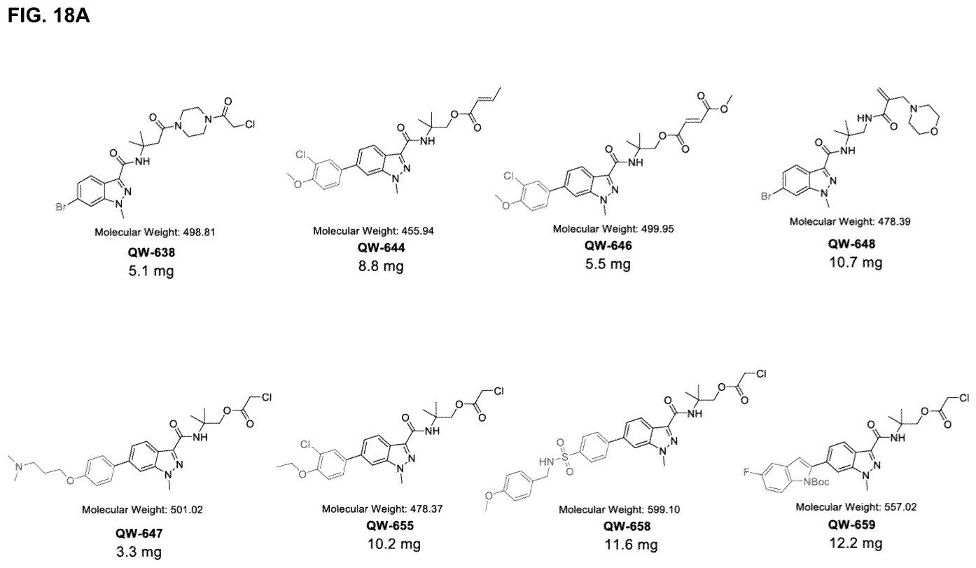

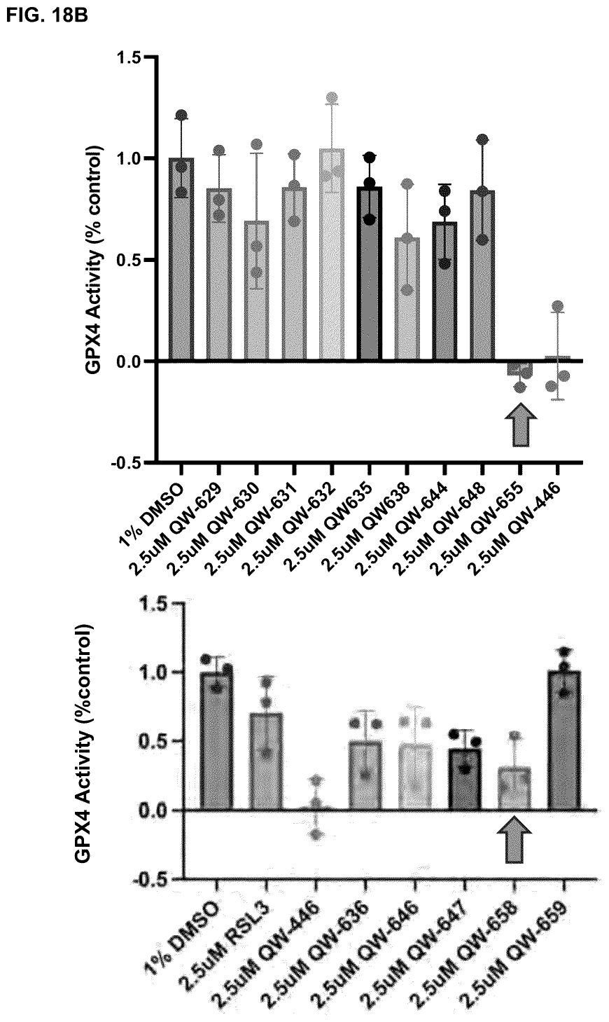

Among additional QW-446 derivatives synthesized (FIG. 15A), QW-594 showed inhibition of GPX4 activity that is comparable to that of QW-446 (FIG. 15B). While additional warheads did not confer improved potency than QW-446 (FIG. 15C), the substitution of water solubilizing groups even led to slightly diminished potency (FIG. 15D). QW-446 analogs with different warheads were also synthesized (FIG. 16A). While QW-446 remained the most potent analog, those alternative warheads were not as active (FIG. 16B). We further tested amide replacement for ester linkage (FIG. 17A). Amide analogs of top leads showed general diminished GPX4 inhibitory activity in vitro (FIG. 17B), while QW-624 was still active in lipid peroxidation assay in HT-1080 cells (FIG. 17C). New compounds with novel warheads, water-solubilizing groups and heterocycles were synthesized (FIG. 18A), among which QW-655 showed the greatest potency (FIG. 18B).

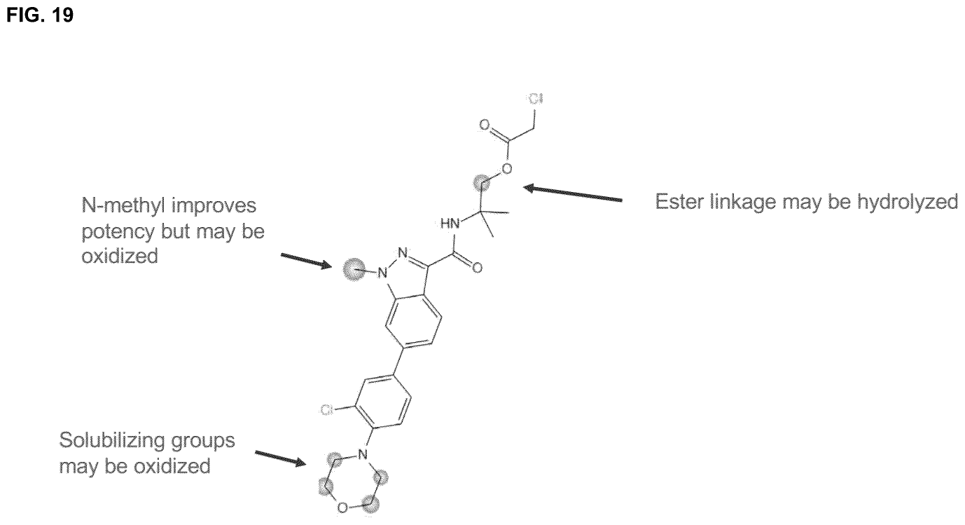

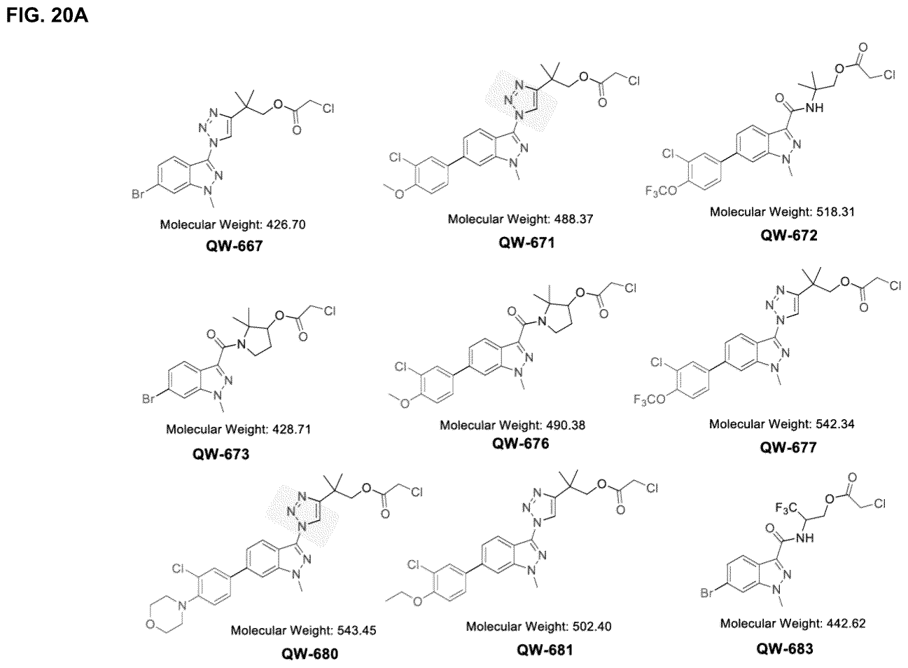

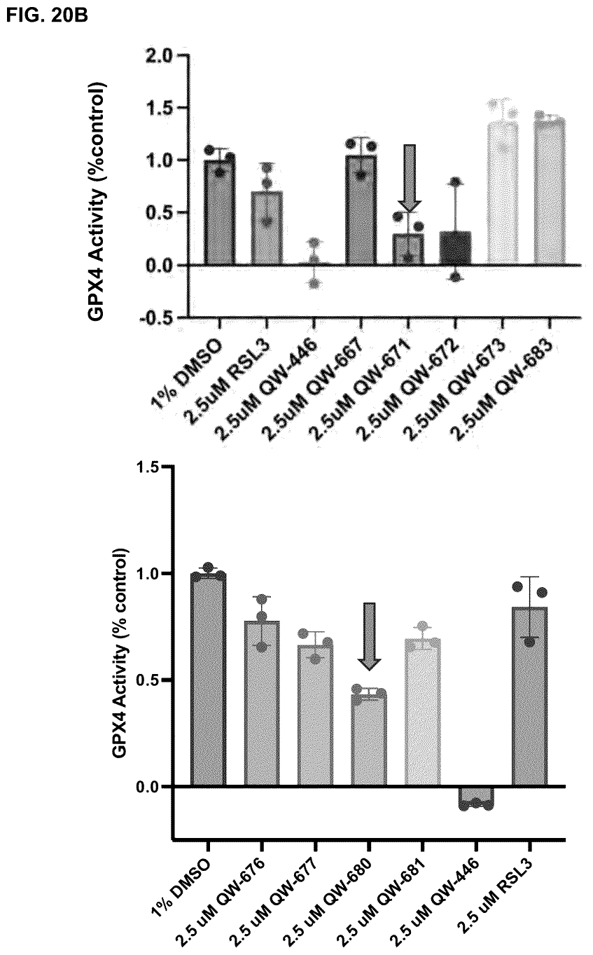

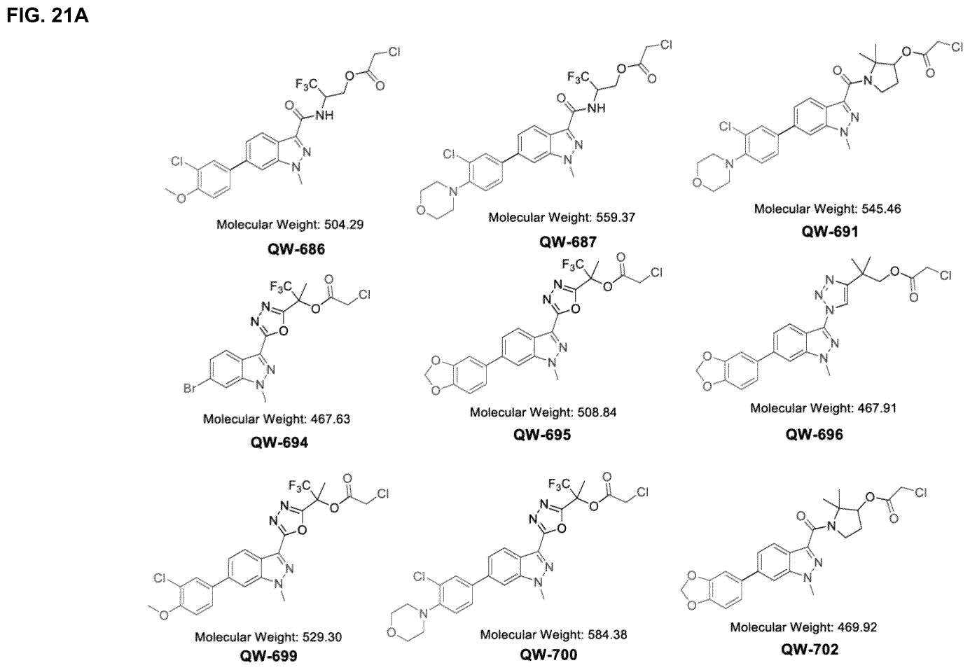

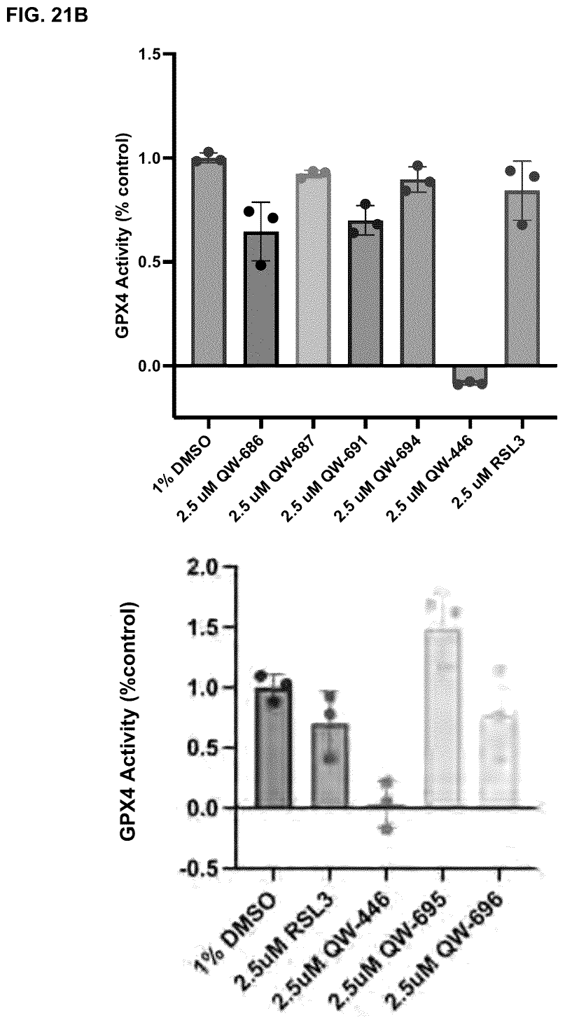

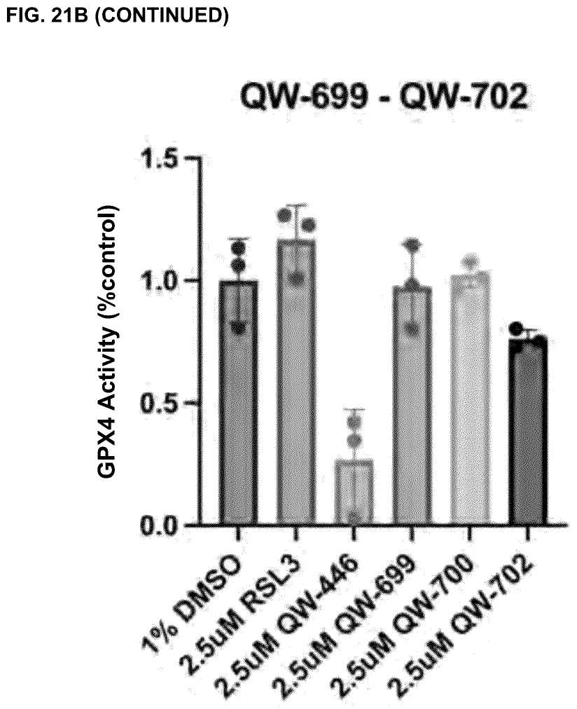

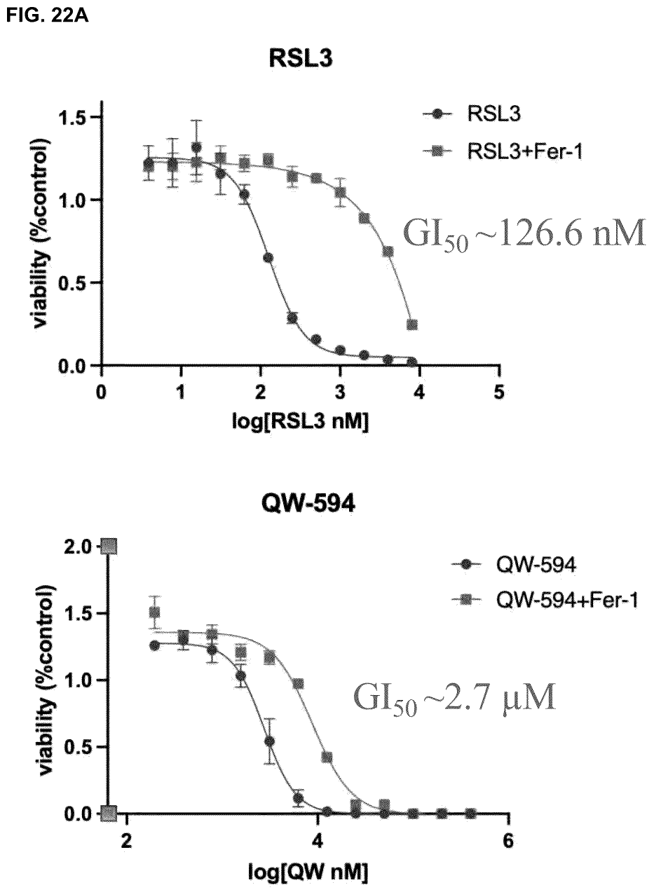

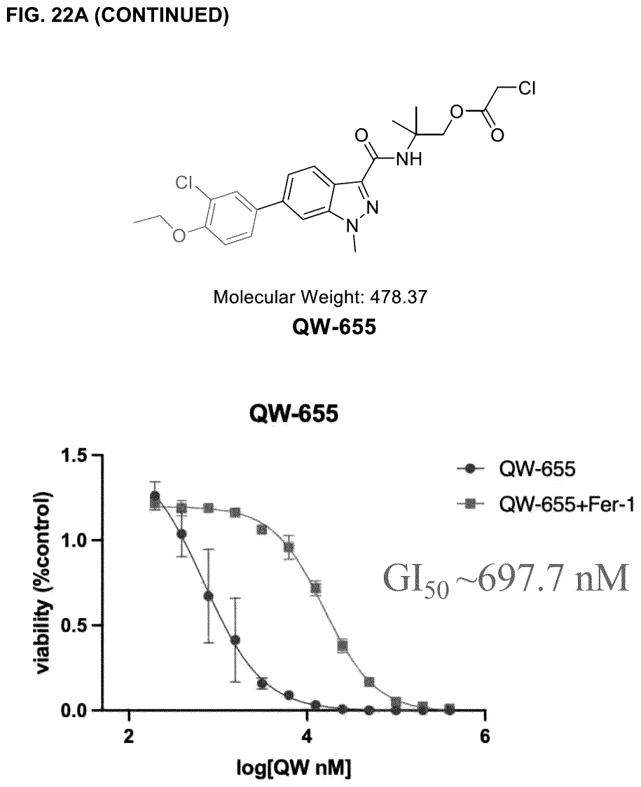

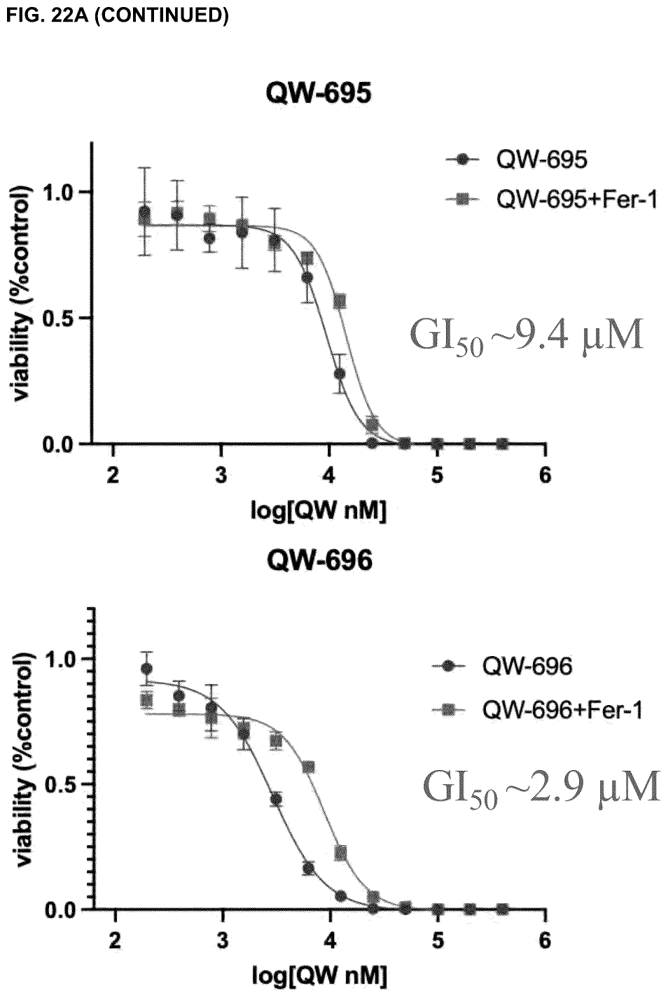

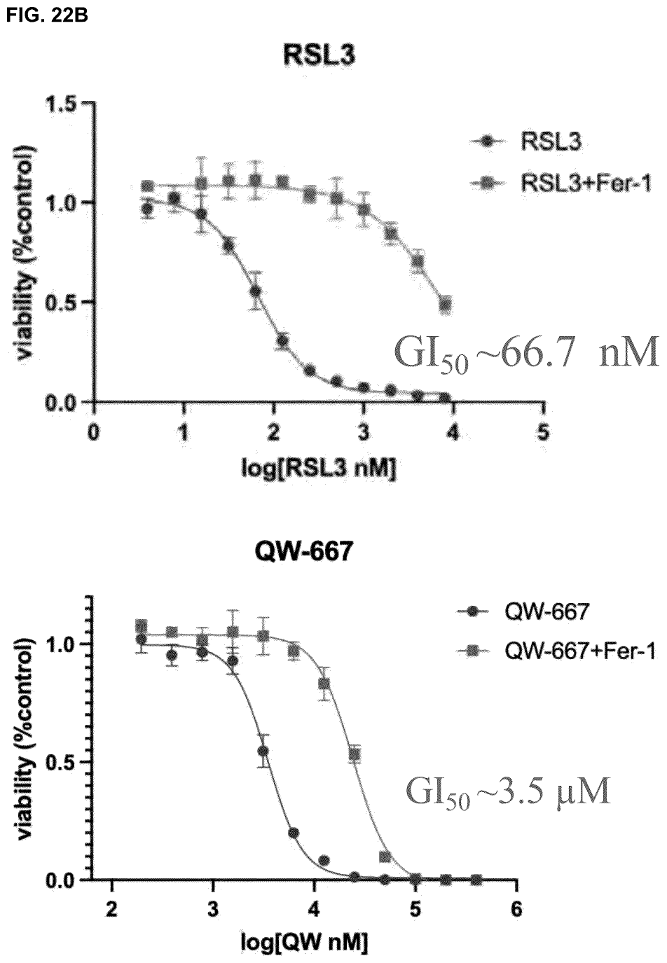

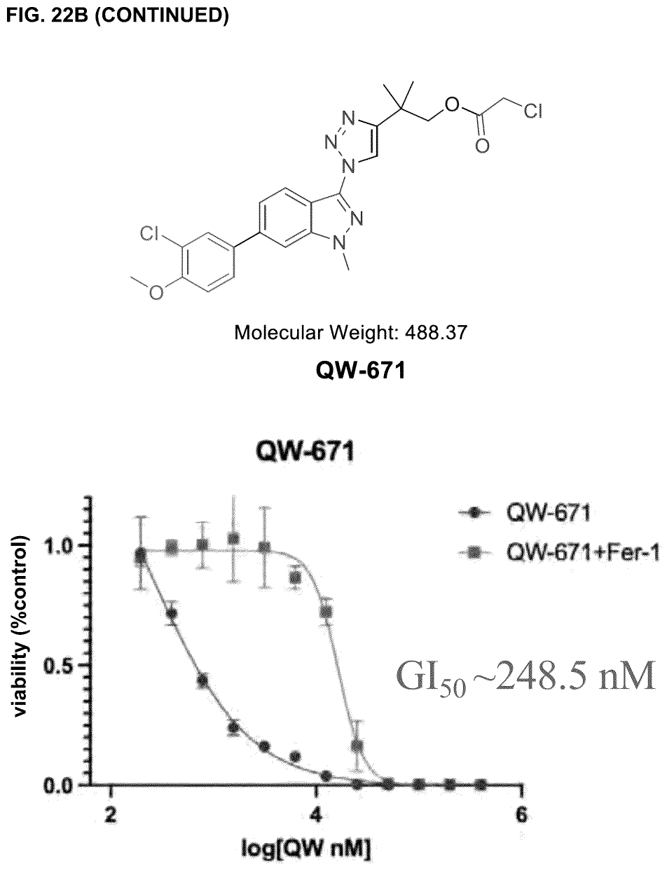

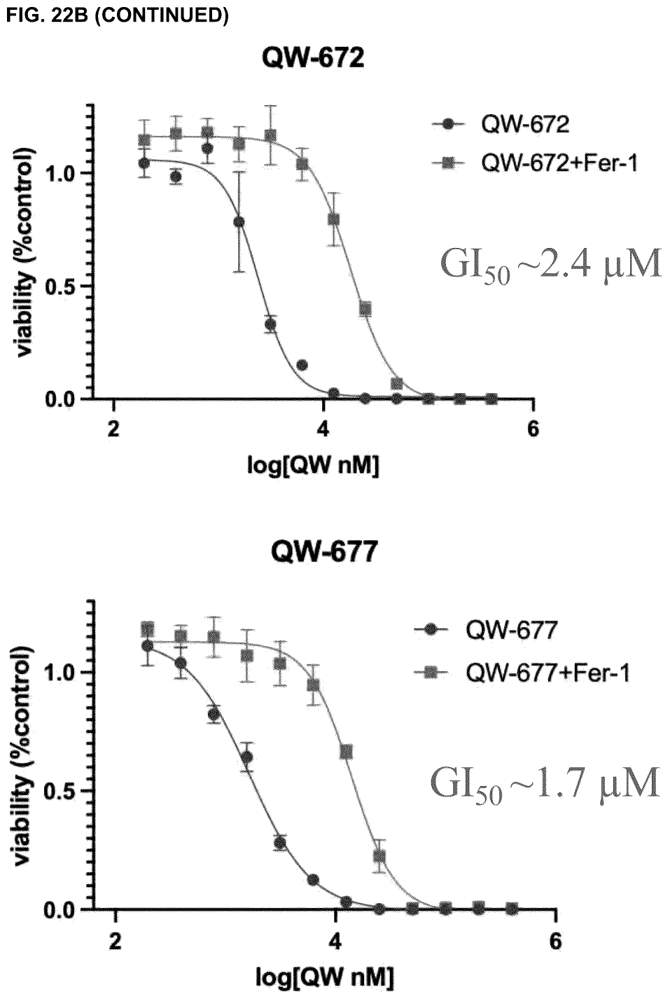

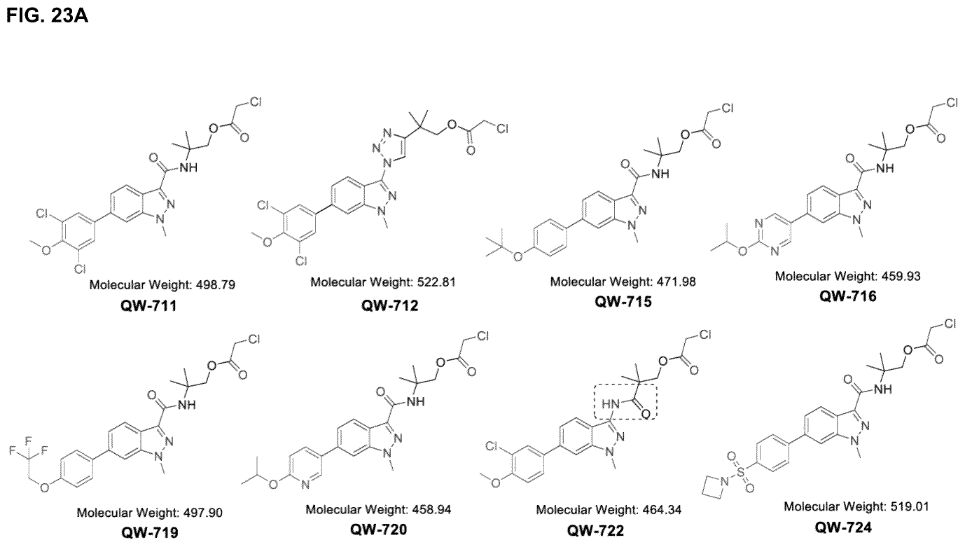

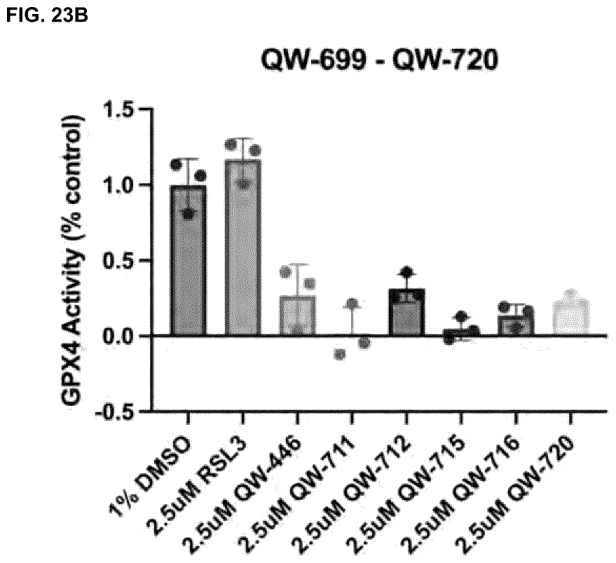

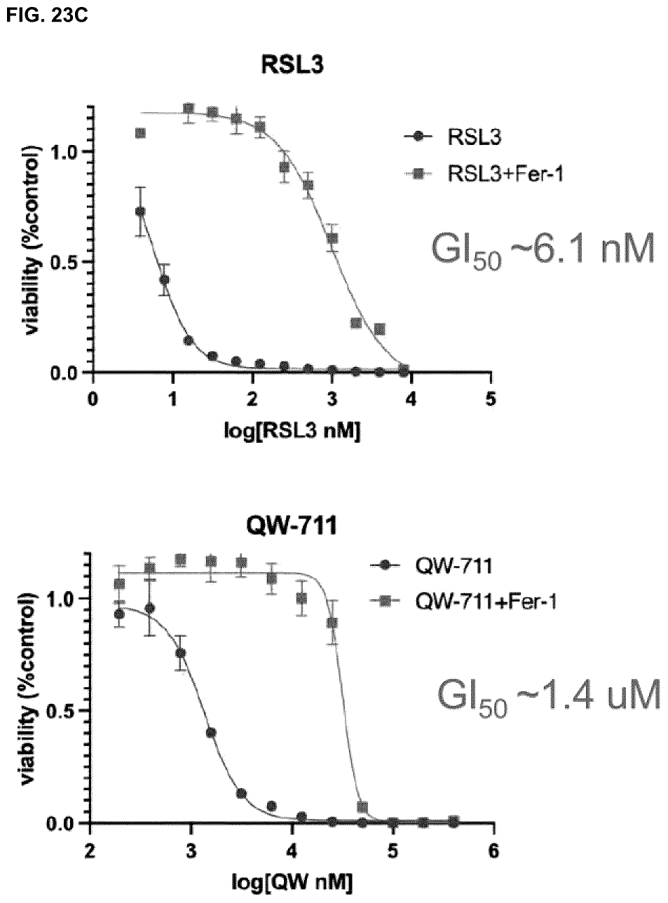

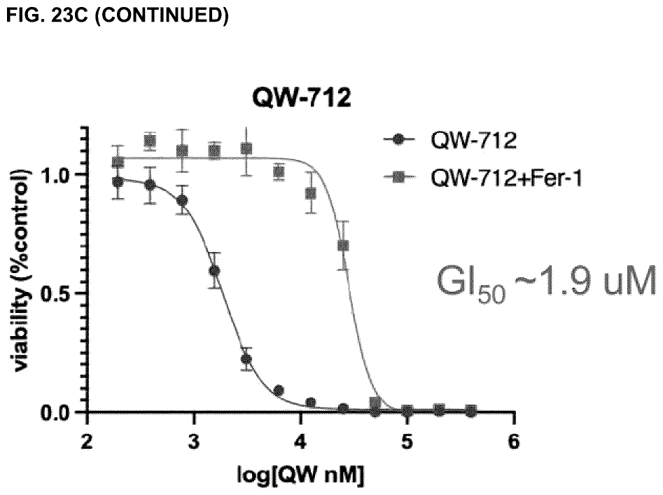

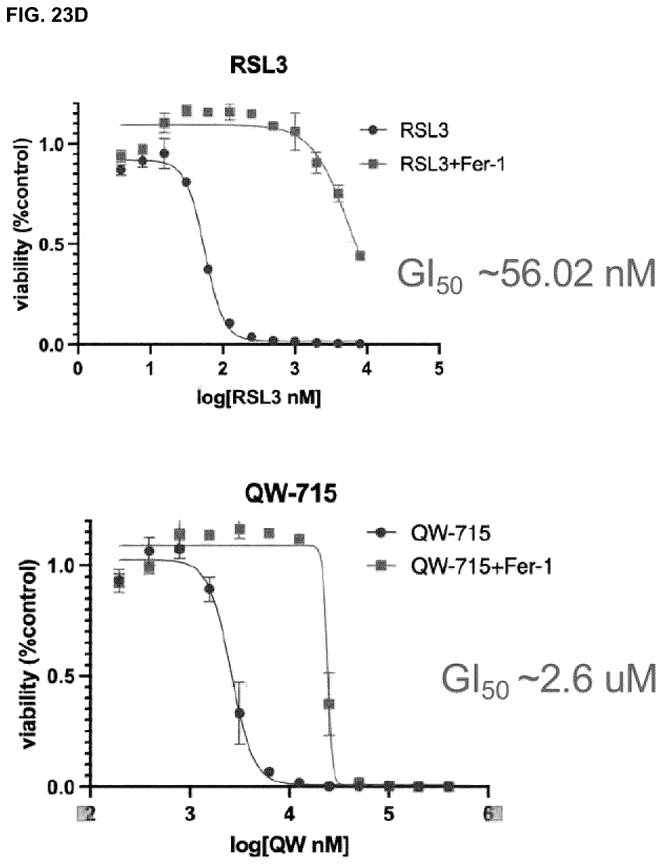

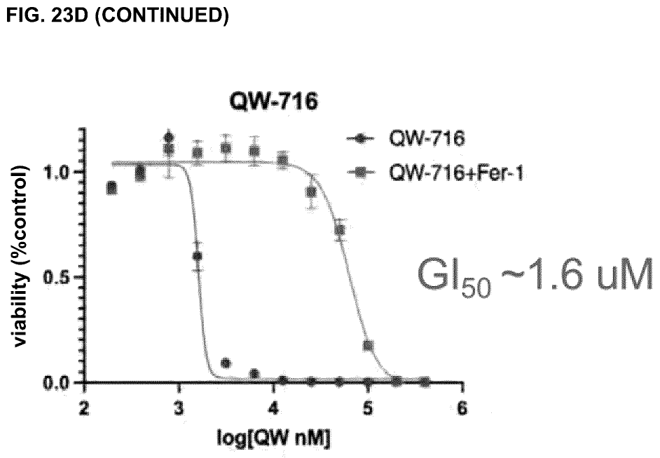

FIG. 19 shows sites of metabolism that predict metabolic liabilities in QW series. Replacement of the amide bond with triazole was tolerated (FIGS. 20A and 20B), and reversing the amide bond in the linker and introduction of cyclic linker and —CF₃ resulted in loss of GPX4 inhibitory activity in vitro (FIGS. 21A and 21B). Both QW-655 and QW-671 induced ferroptosis in moderately resistant HT-1080 fibrosarcoma cells with sub-micromolar GI₅₀ value (FIGS. 22A and 22B). Analogs synthesized by reversing of the amide bond and introduction of novel water-solubilizing groups and heterocycles (FIG. 23A) showed GPX4 inhibitory activity that was comparable to QW-446 (FIG. 23B). QW-711 and QW-712 induced ferroptosis in HT-1080 fibrosarcoma cells but were less potent compared to RSL3 (FIG. 23C), while QW-715 and QW-716 induced ferroptosis in HT-1080 fibrosarcoma cells and were more potent compared to RSL3 (FIG. 23D).

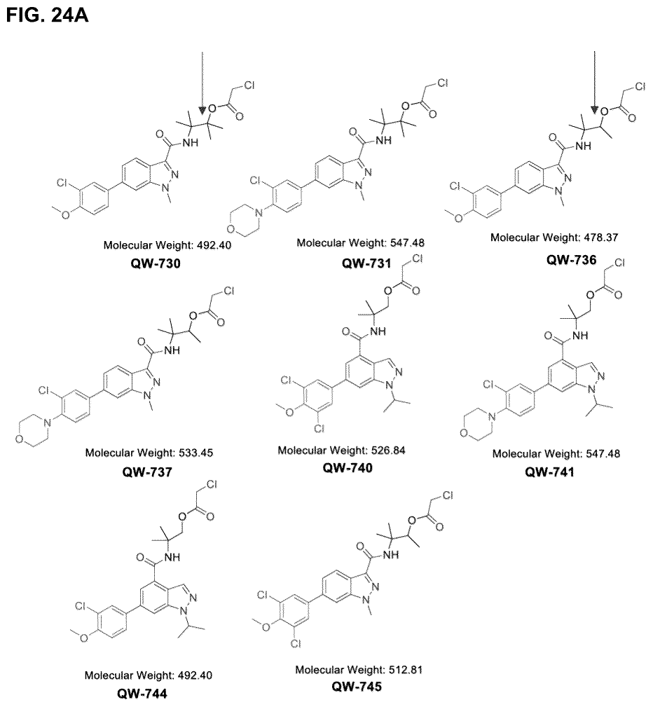

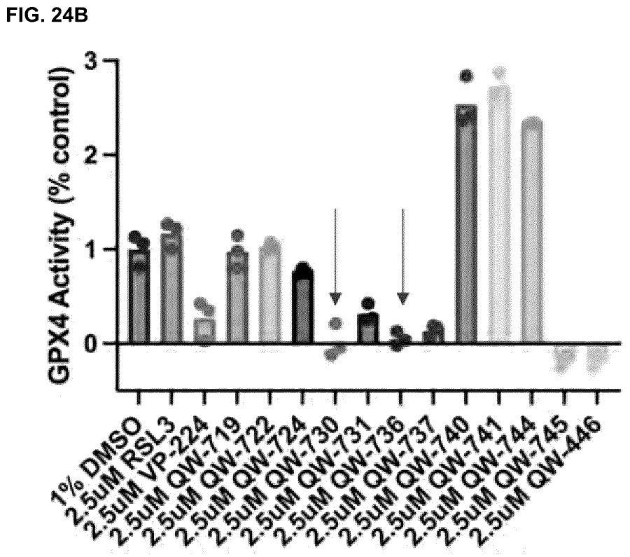

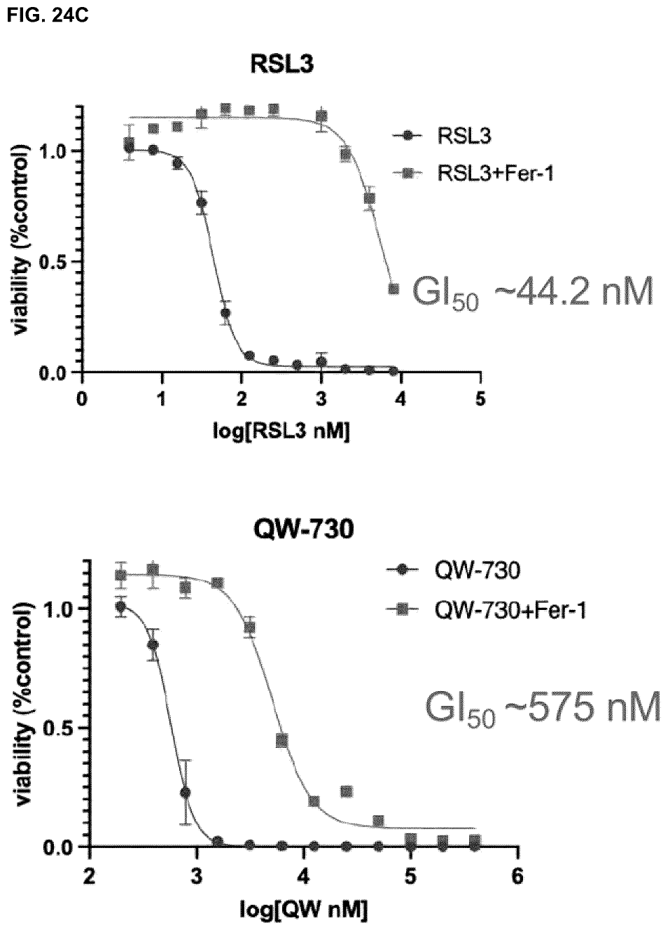

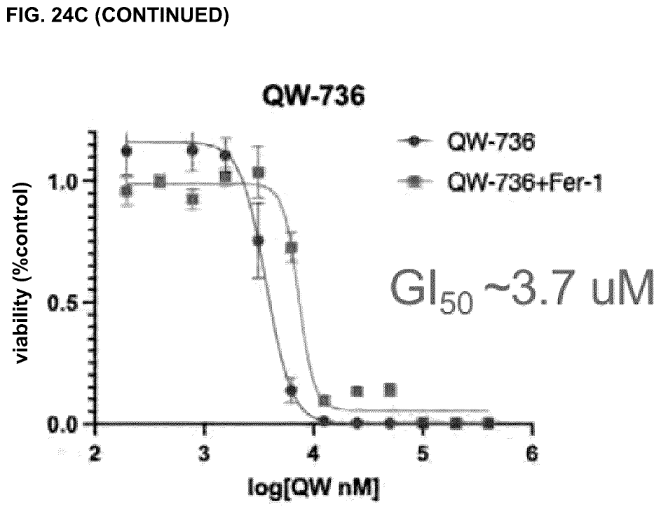

Analogs with secondary and tertiary alcohols (FIG. 24A) showed great potency (FIG. 24B). QW-730 induced ferroptosis in HT-1080 fibrosarcoma cells with sub-micromolar GI₅₀ value (FIG. 24C).

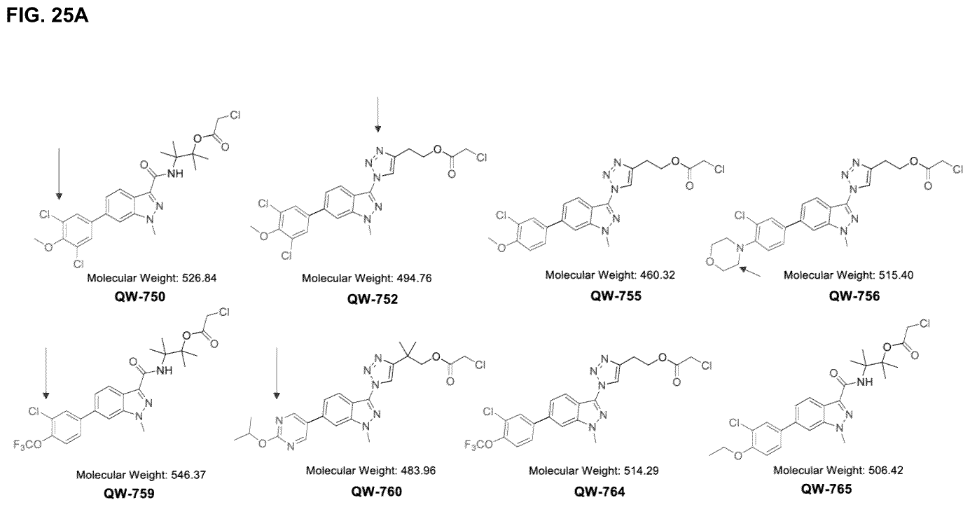

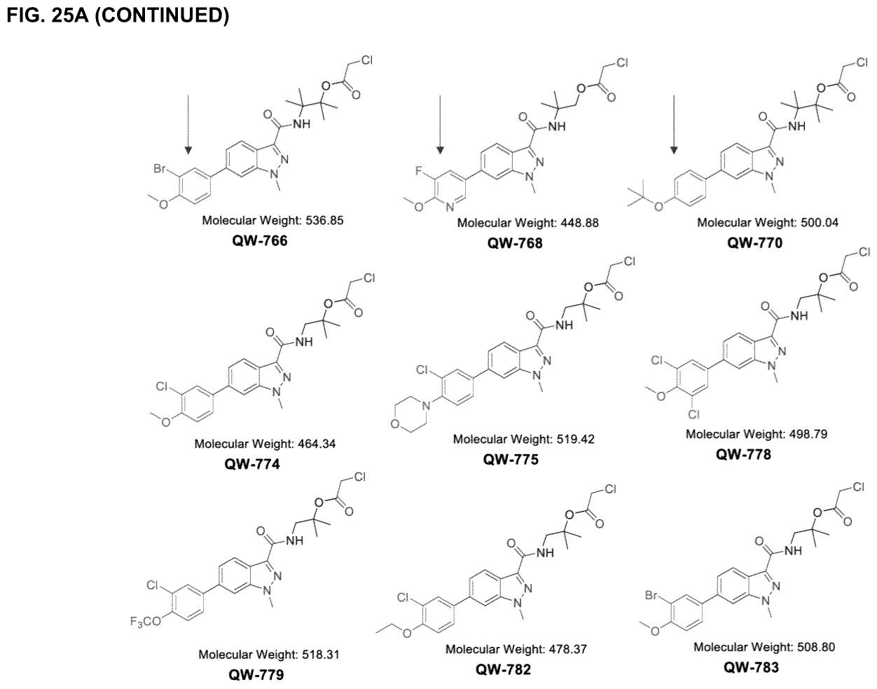

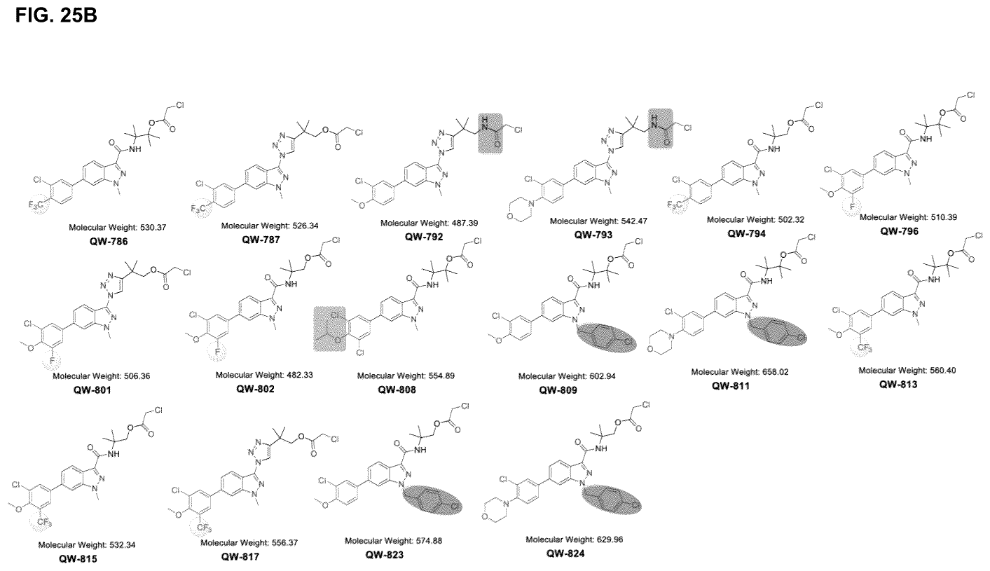

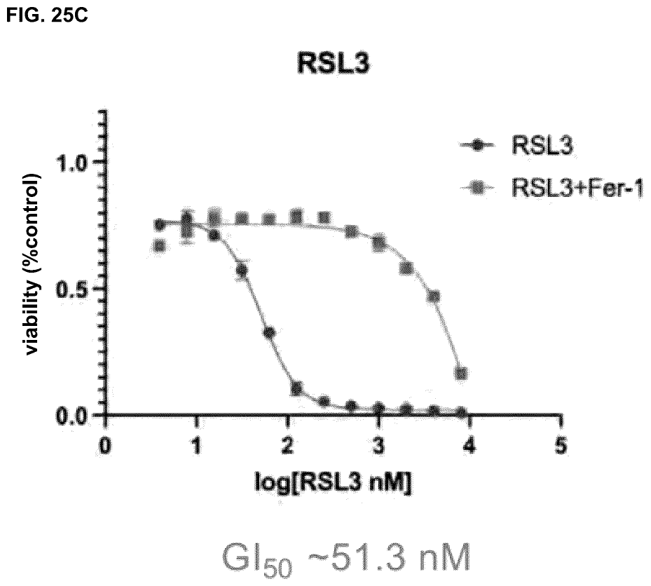

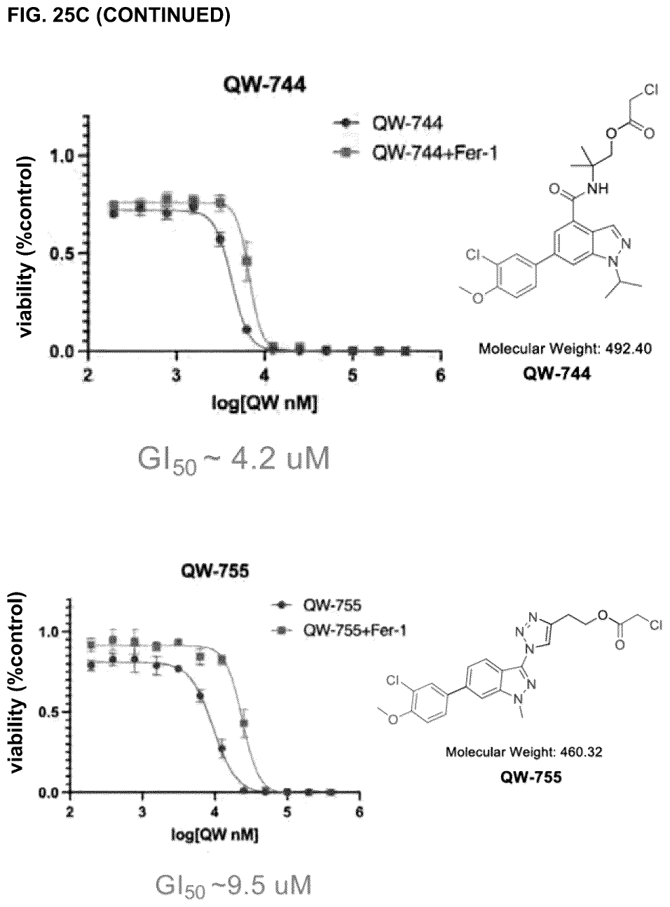

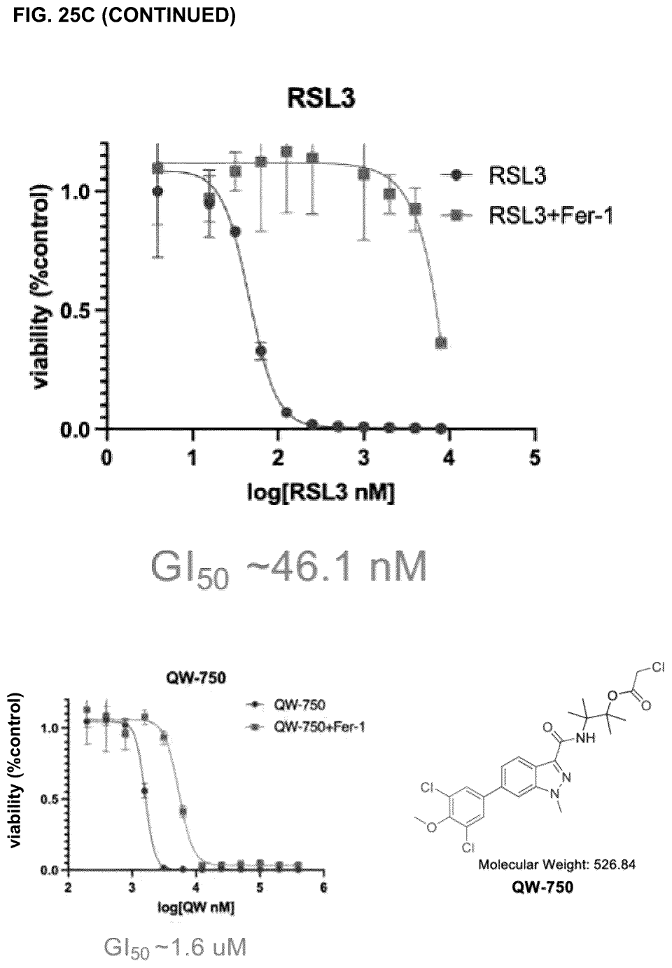

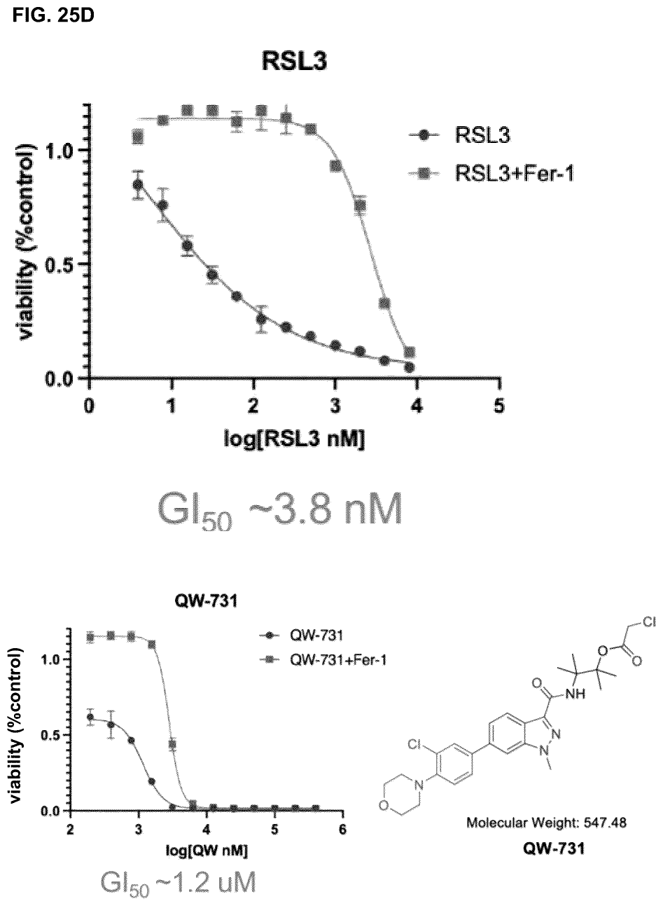

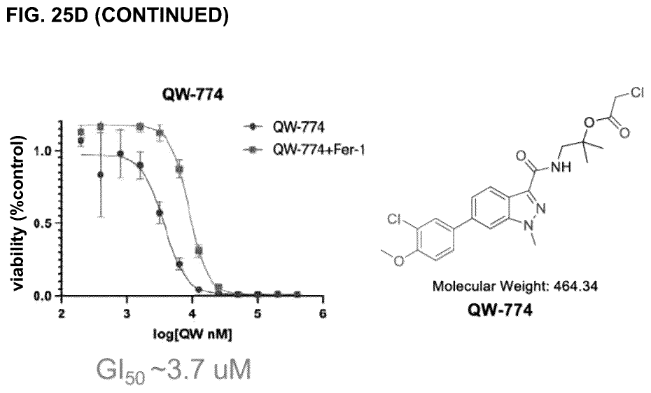

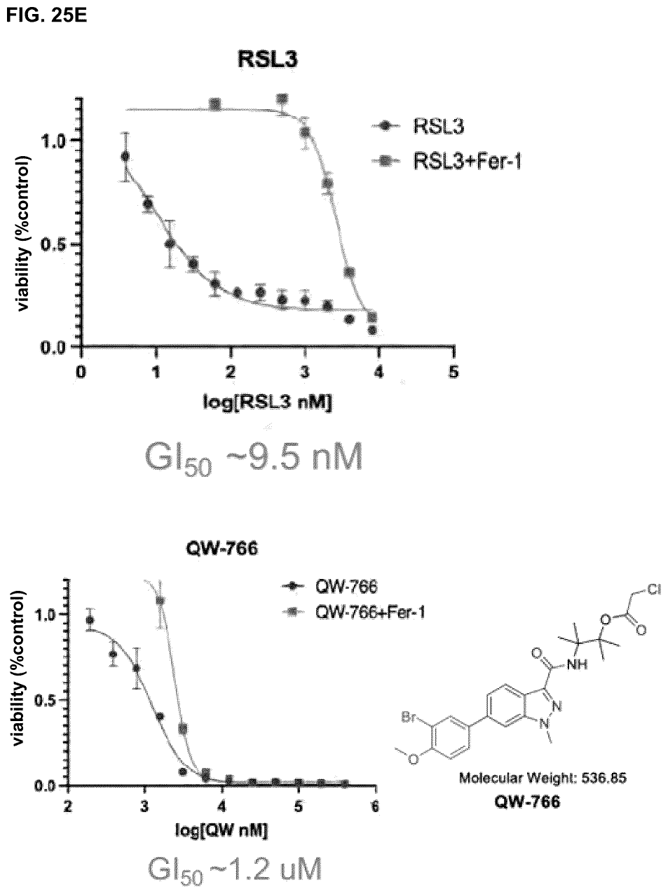

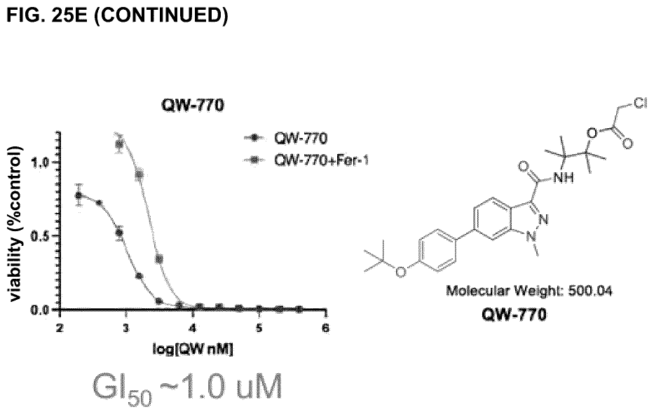

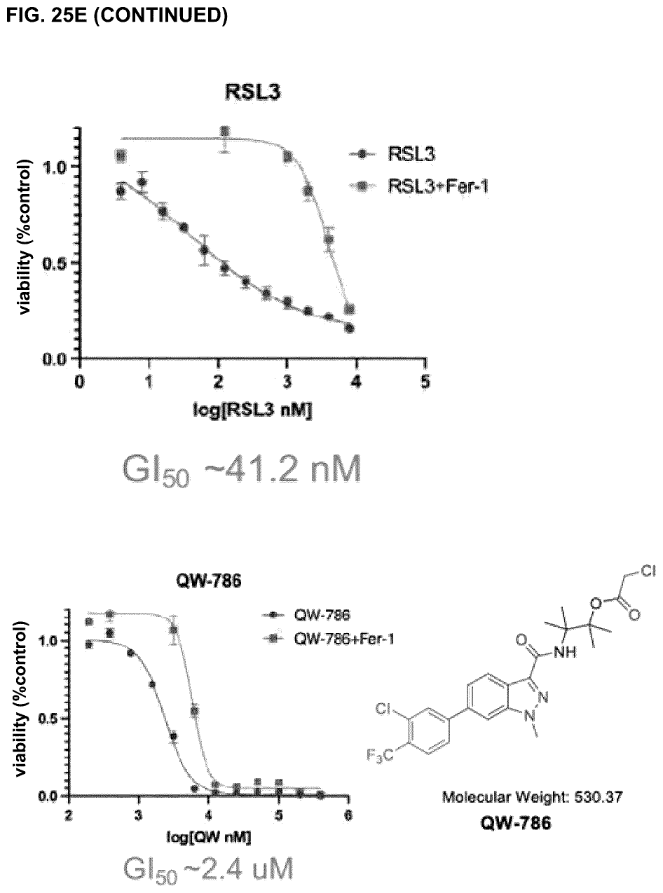

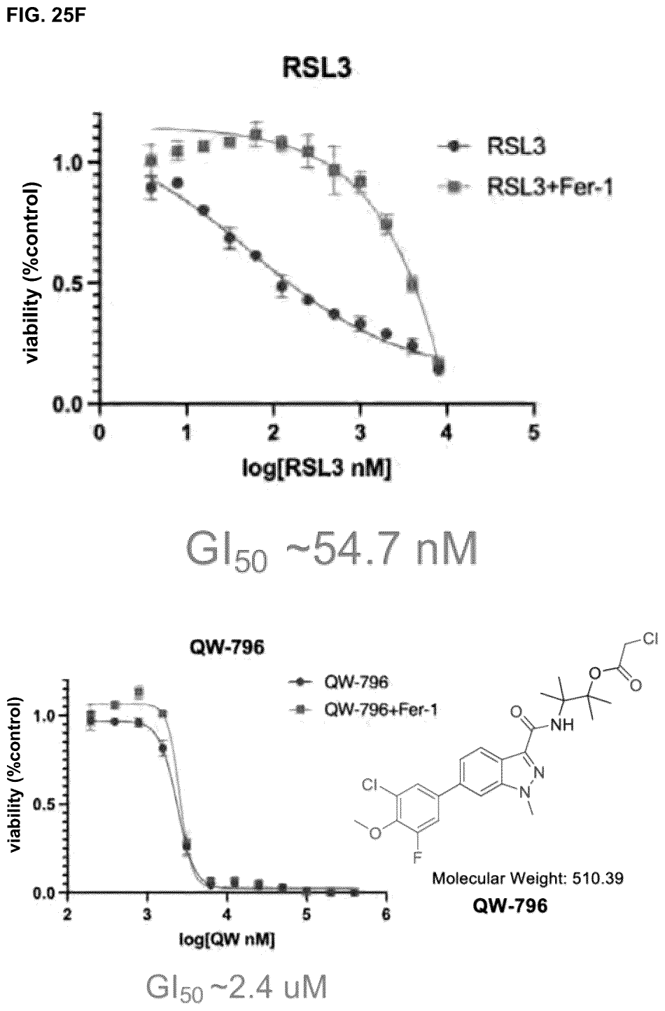

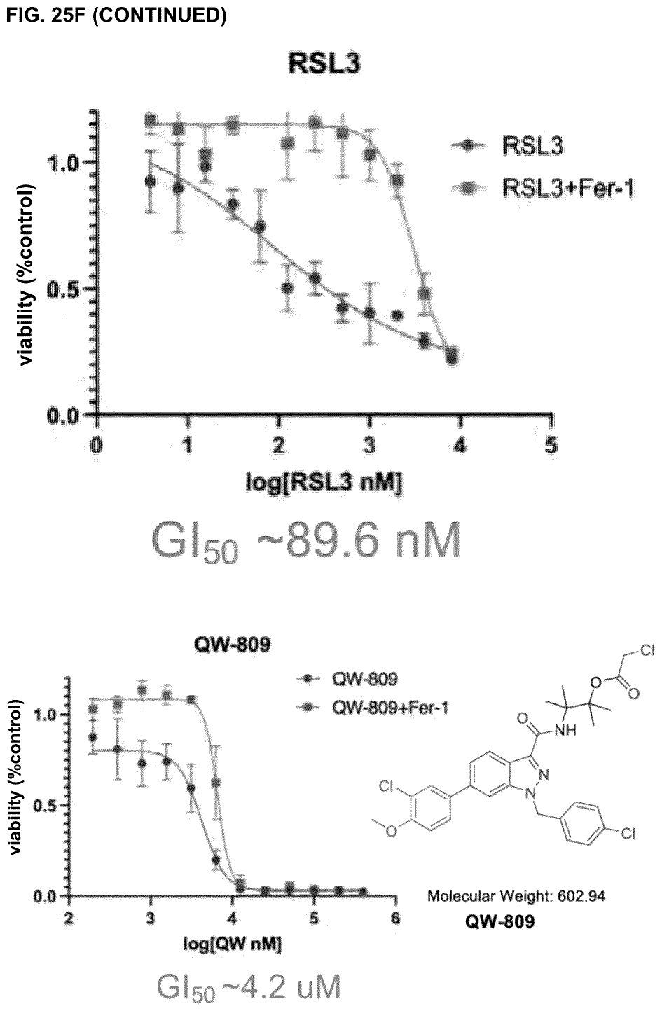

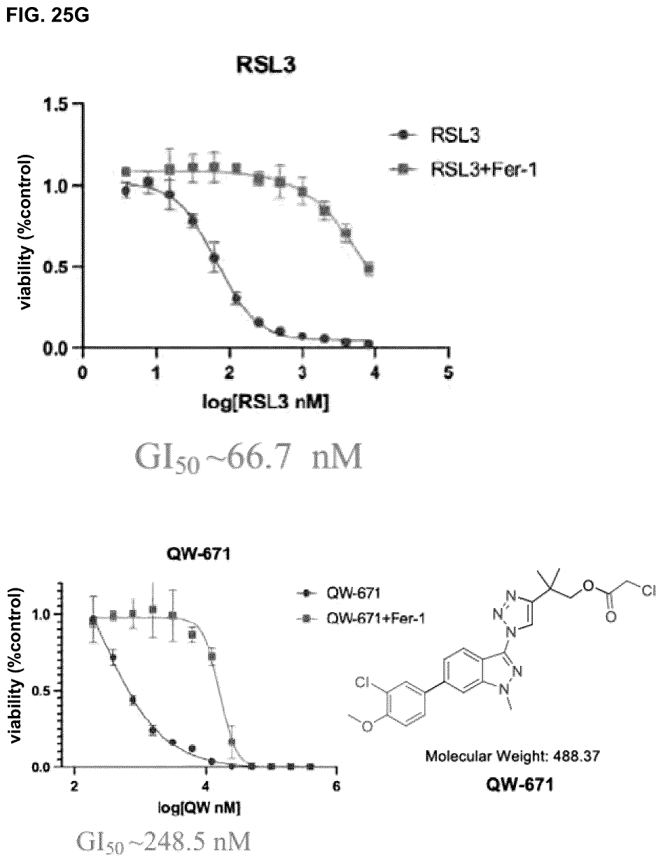

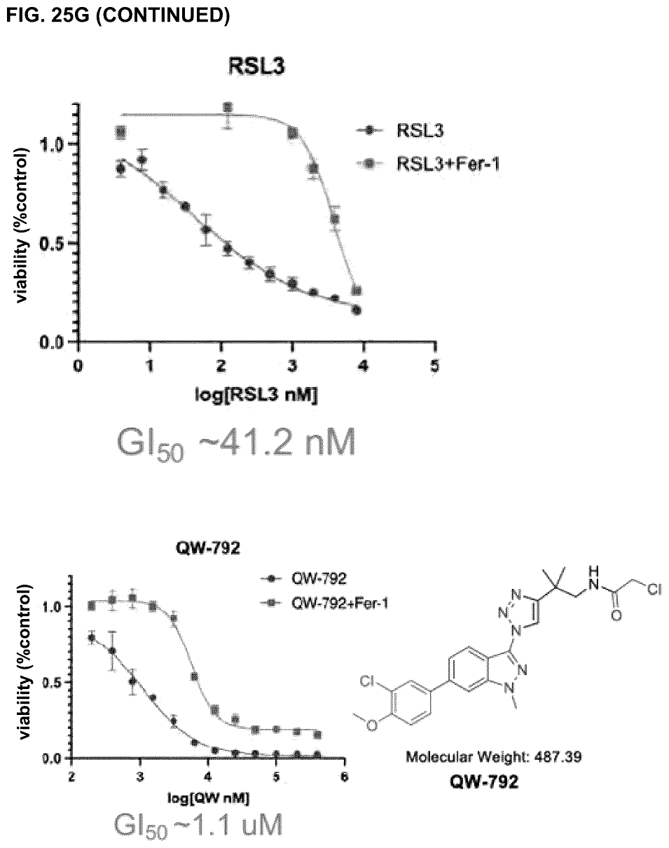

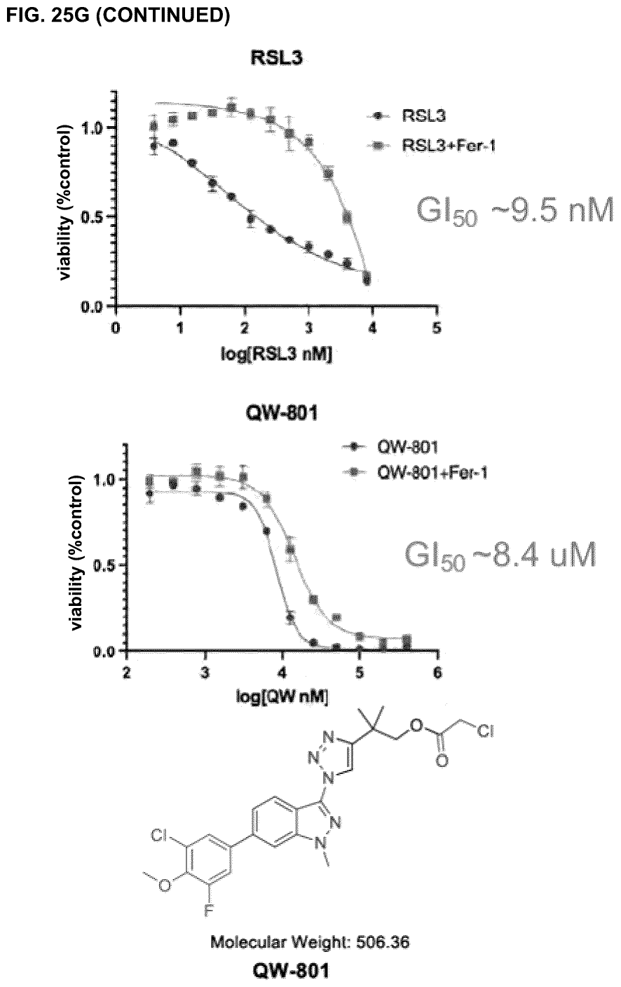

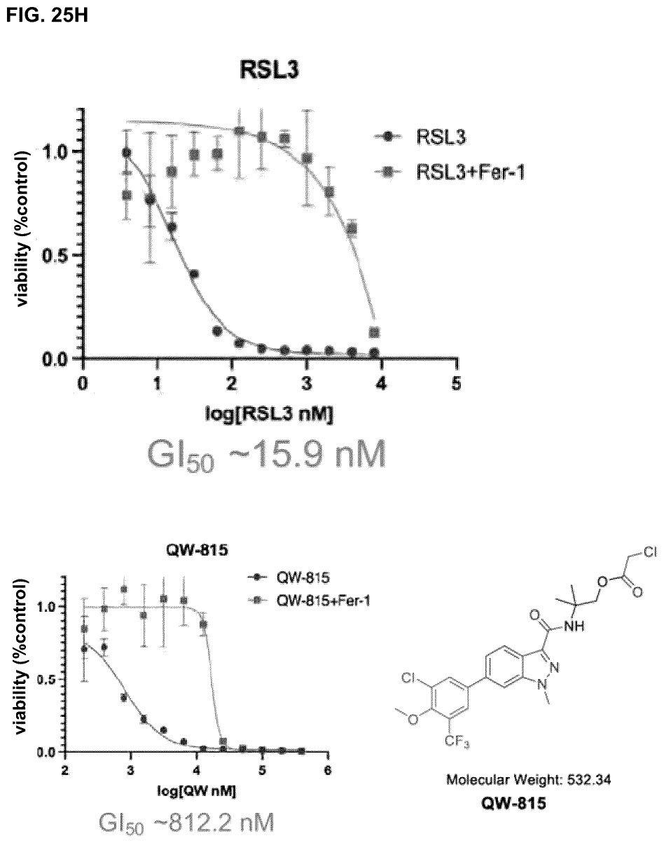

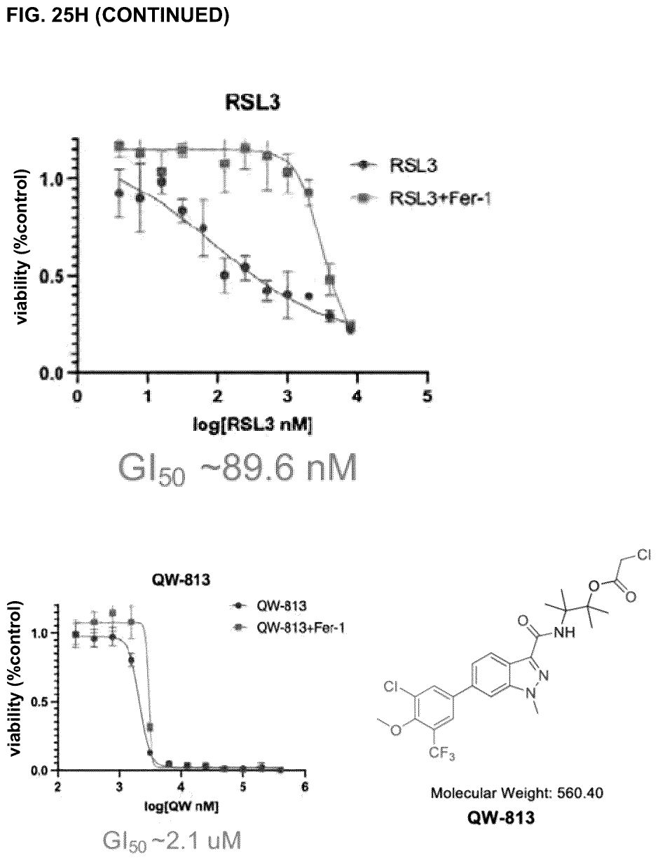

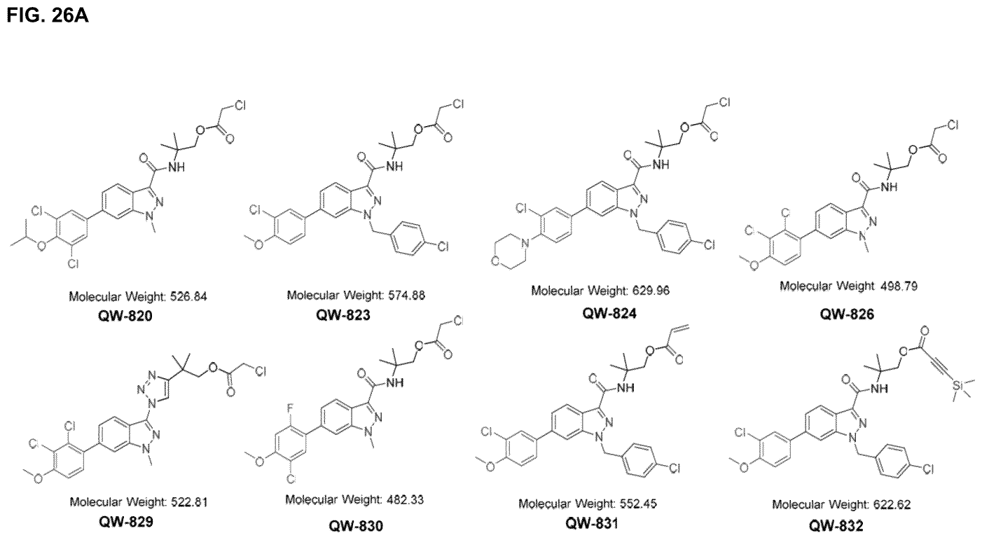

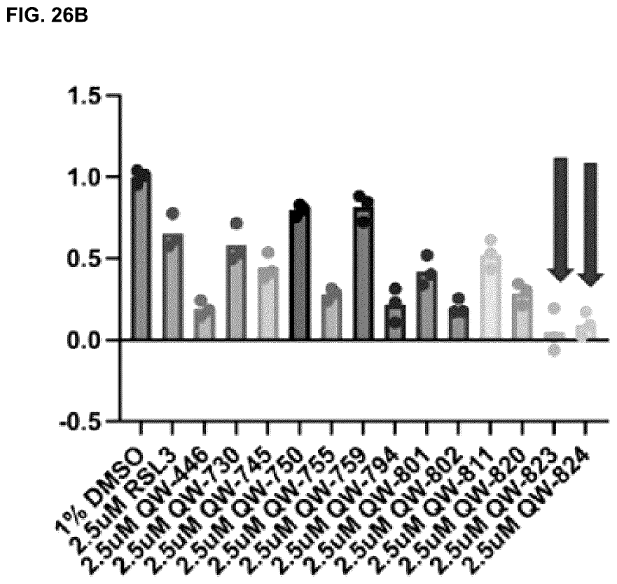

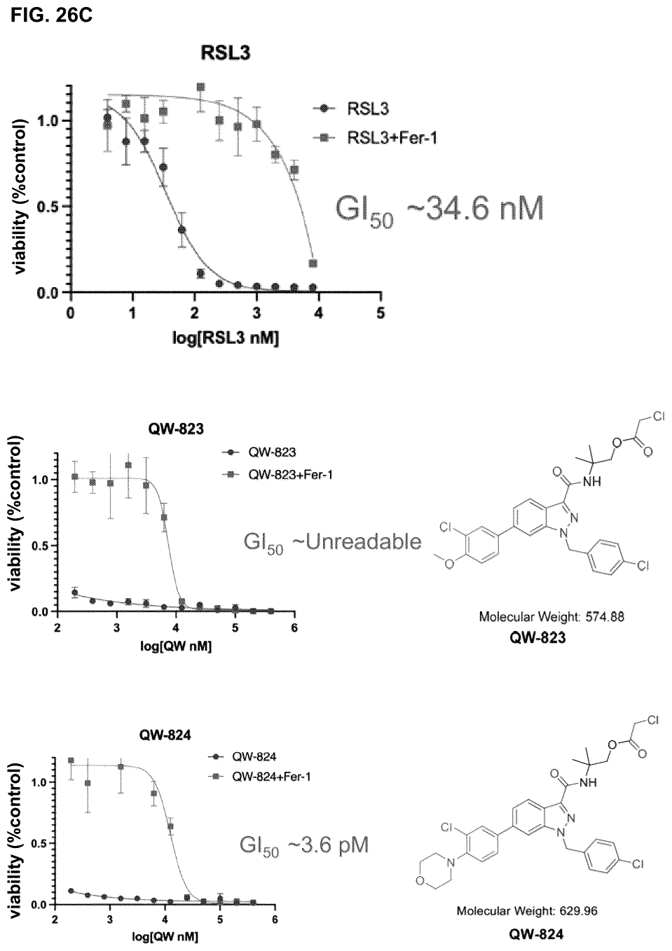

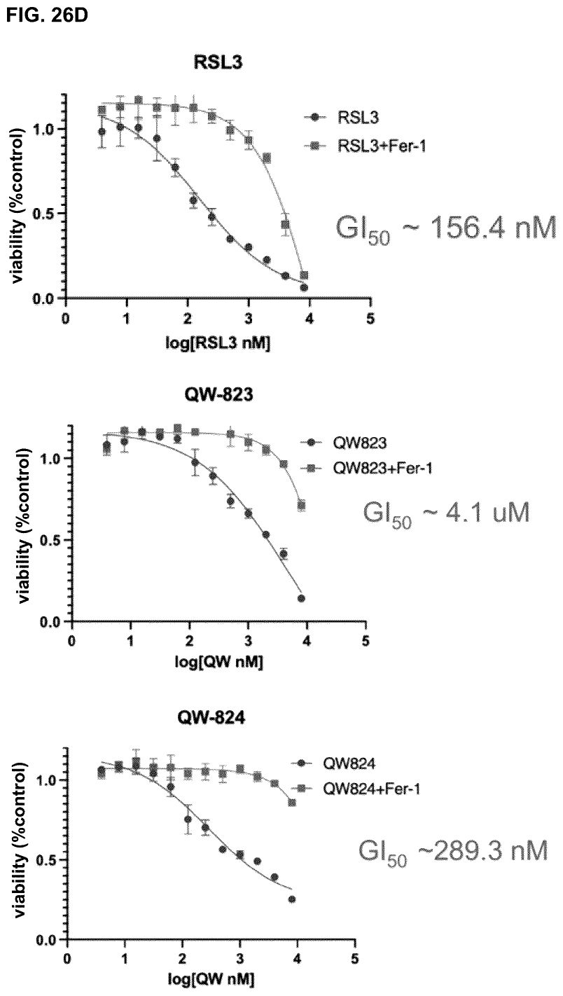

New analogs with diverse linkers and heterocycles were also synthesized to improve metabolic and plasma stability (FIG. 25A). Modifications at different sites were tested for greater potency and metabolic stability (FIG. 25B). QW-750 induce ferroptosis in HT-1080 fibrosarcoma cells with moderate potency (FIG. 25C). Analogs with new heterocycle and stabilized linker induce ferroptosis in HT-1080 fibrosarcoma cells with reduced potency (FIG. 25D). Tetramethyl-derived linker caused the loss of selectivity for ferroptosis (FIG. 25E). Analogs with more electron-withdrawing characters or bulkier group on N-site did not induce ferroptosis (FIG. 25F). Replacing ester to amide or introducing fluorine resulted in loss of selectivity and potency (FIG. 25G). Introducing CF₃ group in the classic scaffold induced ferroptosis in HT-1080 cells with sub-micromolar GI₅₀ values, indicating that strong electron-withdrawing group (EWG) such as the CF₃ group may improve the metabolic stability (FIG. 25H). Among additional new analogs (FIG. 26A), QW-823 and QW-824 showed greater GPX4 inhibitory activity than QW-446 (FIG. 26B). QW-823 and QW-824 also showed good potency in cell and selectivity to GPX4 (FIG. 26C), which diminished at lower concentration (FIG. 26D). Docking of QW-823 and QW-824 onto WT GPX4 is shown in FIG. 26E.

### Example 5

### Optimization Based on RSL3 Scaffold

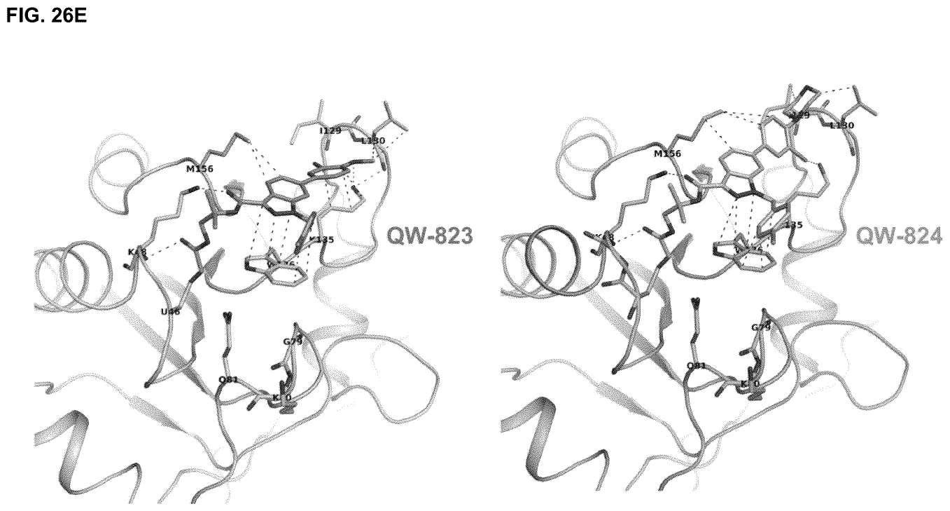

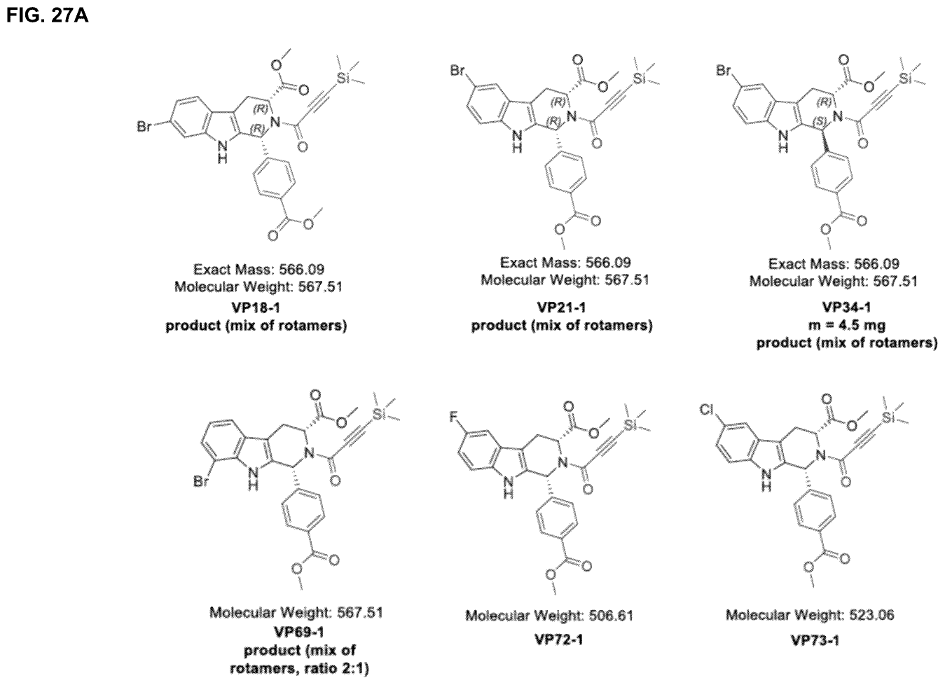

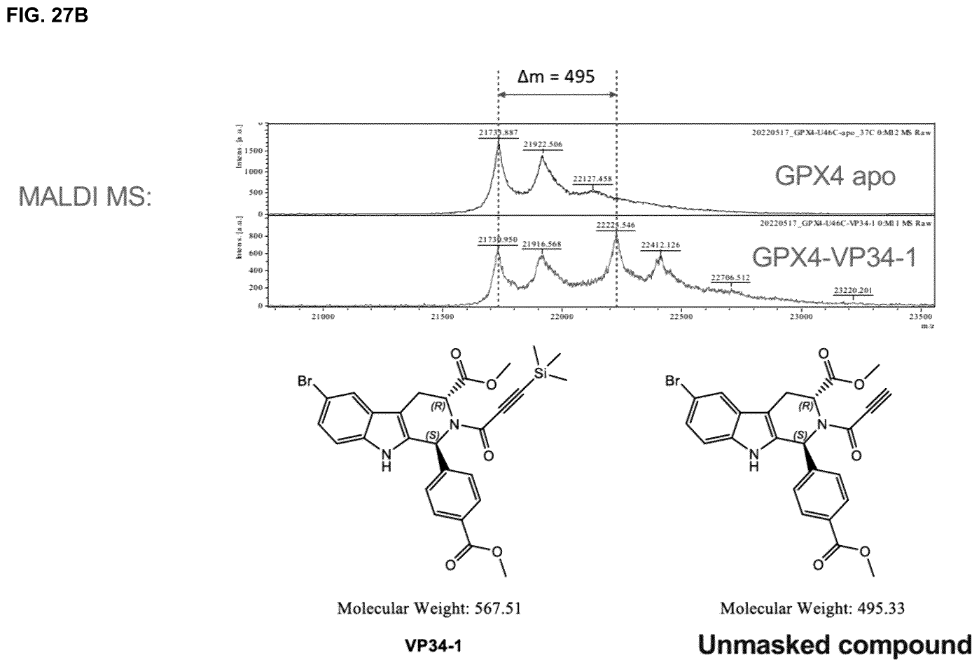

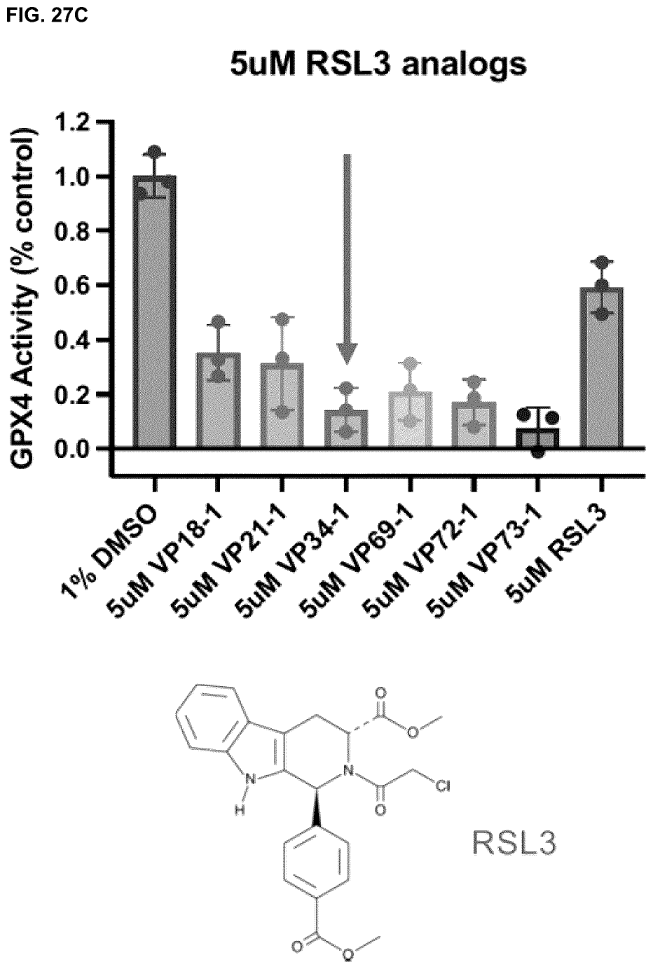

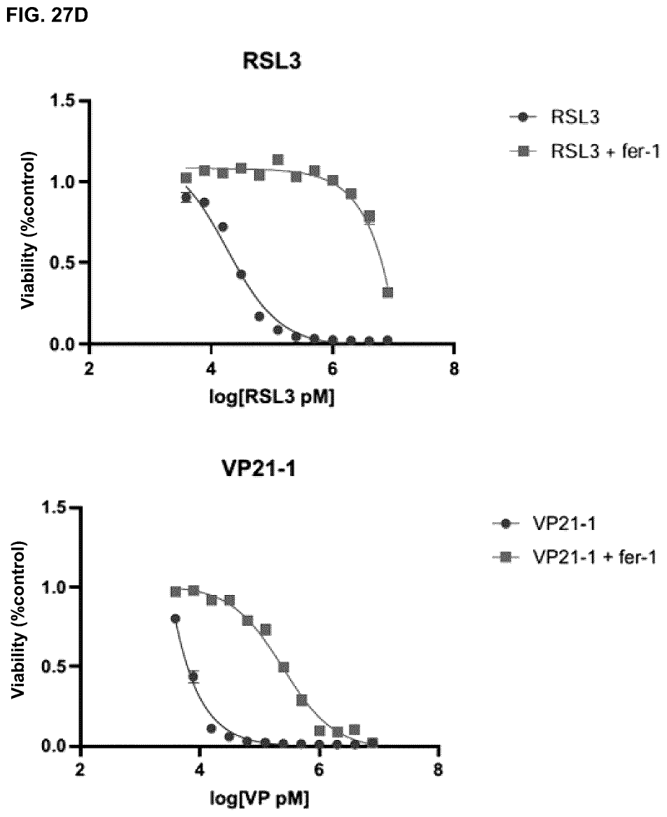

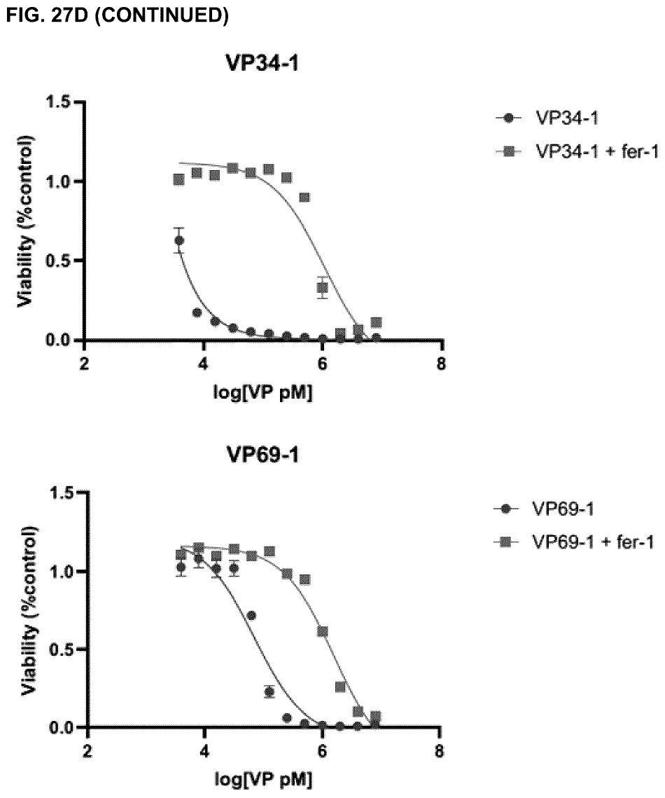

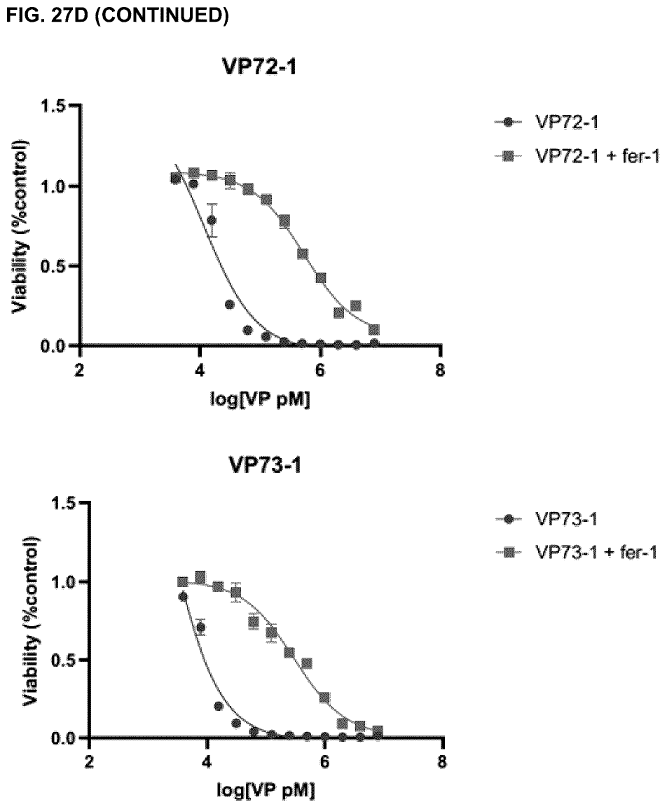

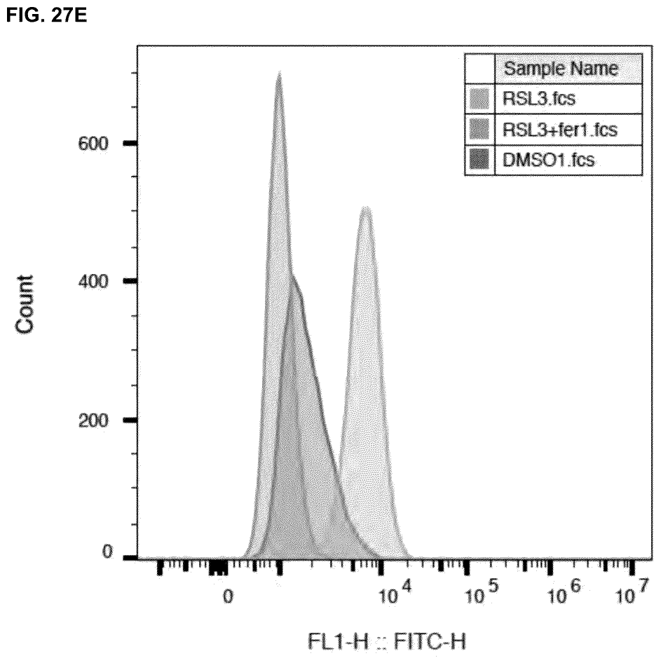

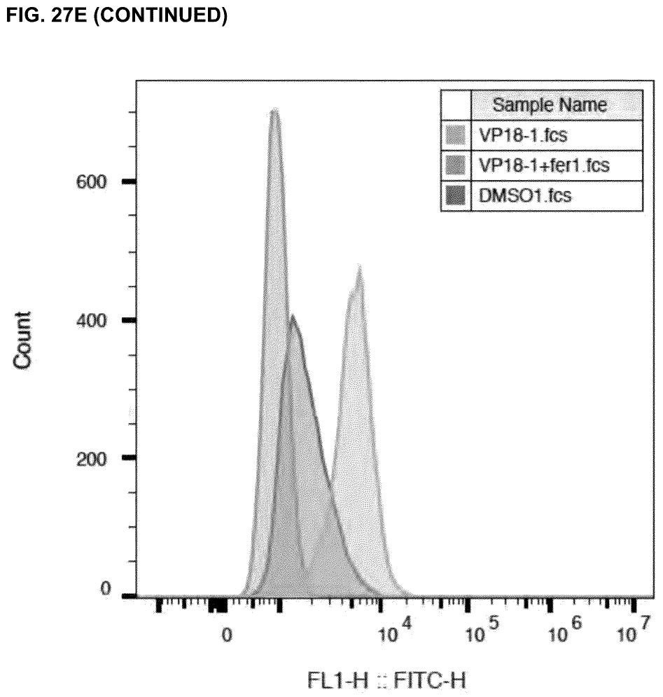

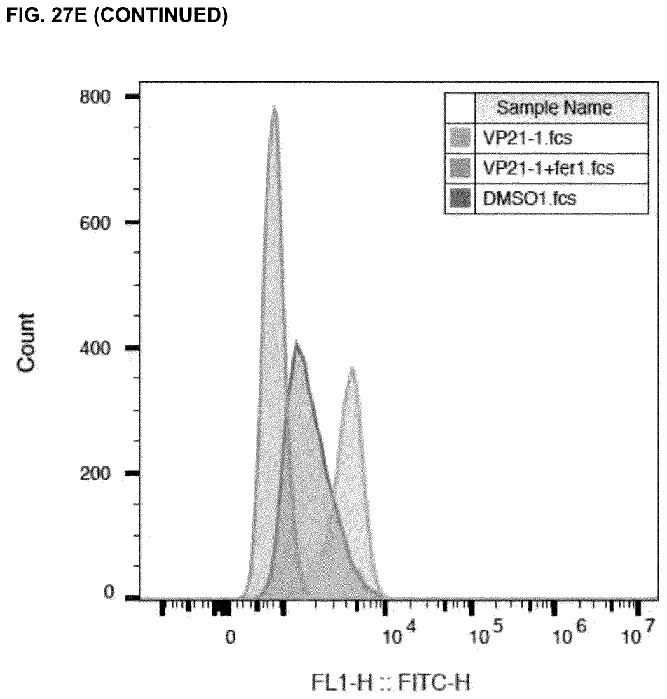

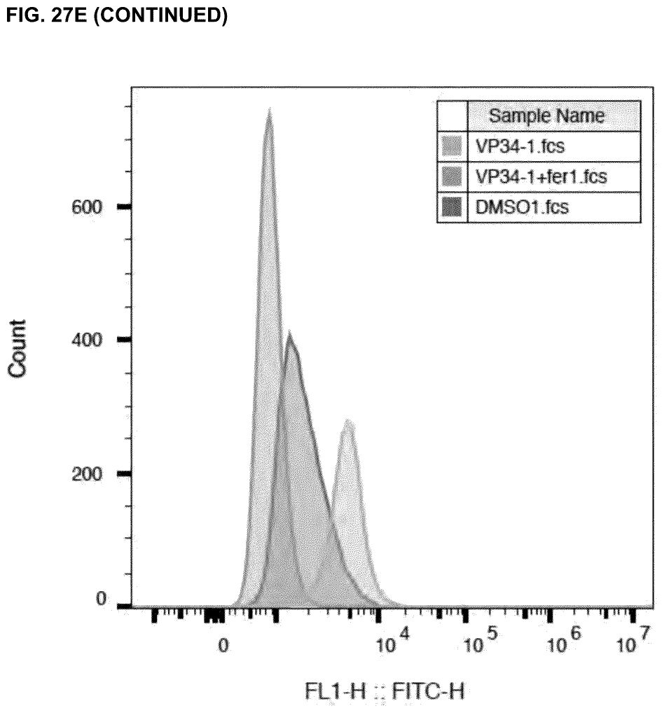

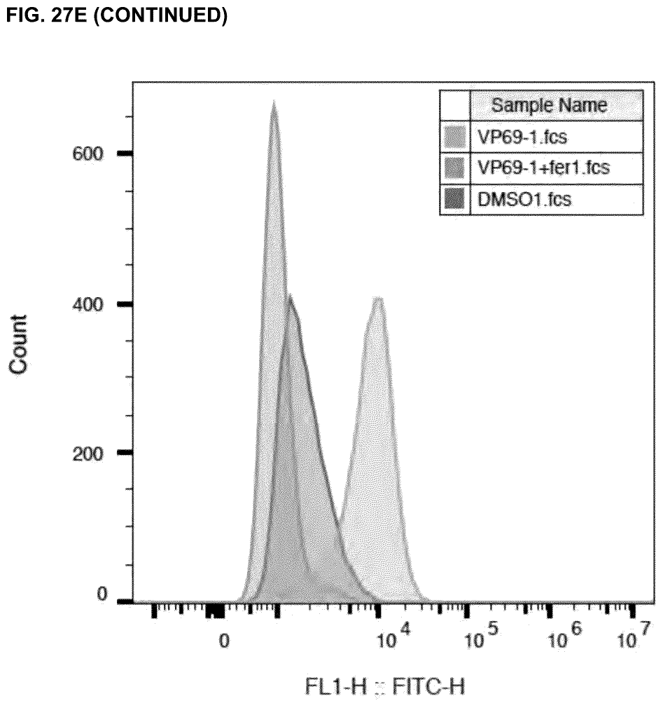

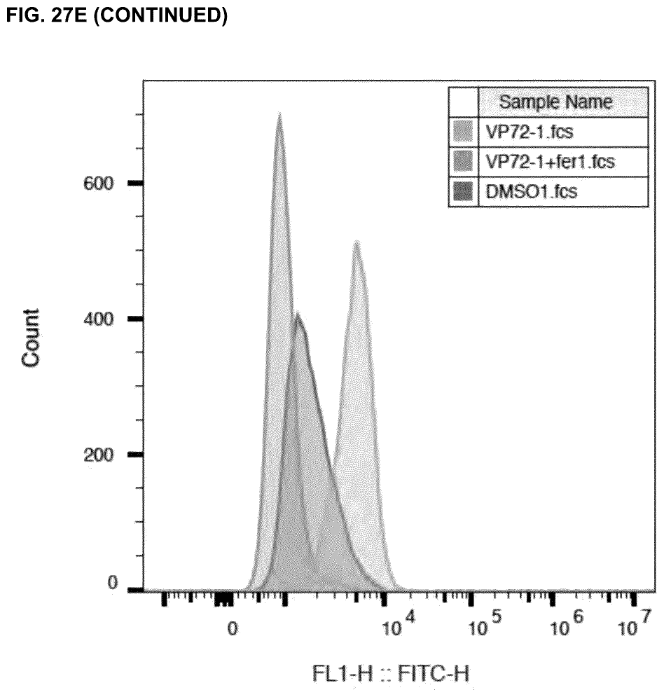

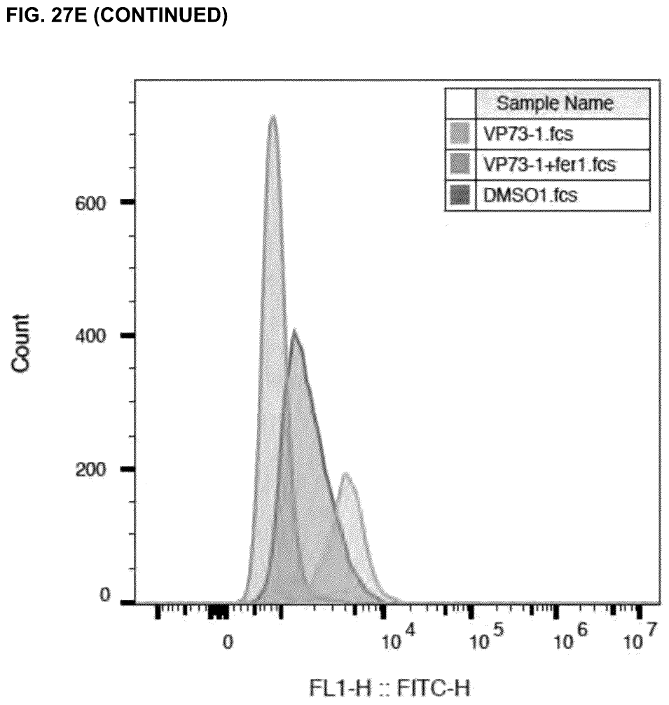

RSL3 is a well-known, cell-active GPX4 inhibitor. It covalently binds GPX4 via an electrophilic warhead chloroacetamide. However, the reactive nature of this inhibitor results in reduced selectivity and drug-like stability. Thus, investigation of a variety of electrophiles as warheads was a good starting point for the development of improved RSL3 analogs as GPX4 inhibitors. We examined the replacement of the chloroacetamide warhead with less reactive electrophiles (FIG. 27A). Using mass spectrometry, we found that protecting group trimethylsilyl (TMS) leaves before masked propiolamide analogs of RSL3 bind to GPX4 (FIG. 27B). The installation of a propiolamide warhead with masked reactivity using a terminal silyl protecting group that can be removed in situ and the introduction of a halogen on the indole ring, resulted in significant improved GPX4 inhibitory activity in a lysate-based assay when compared to RSL3 (FIG. 27C). Masked propiolamide analogs of RSL3 also induced ferroptosis (FIG. 27D) and ferrostatin-rescuable lipid peroxidation in HT-1080 cells (FIG. 27E).

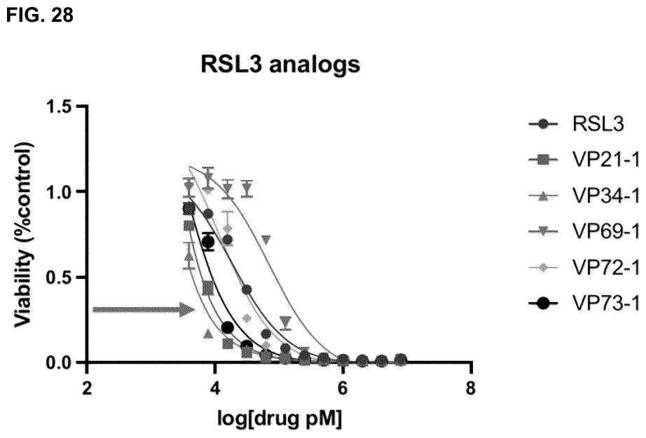

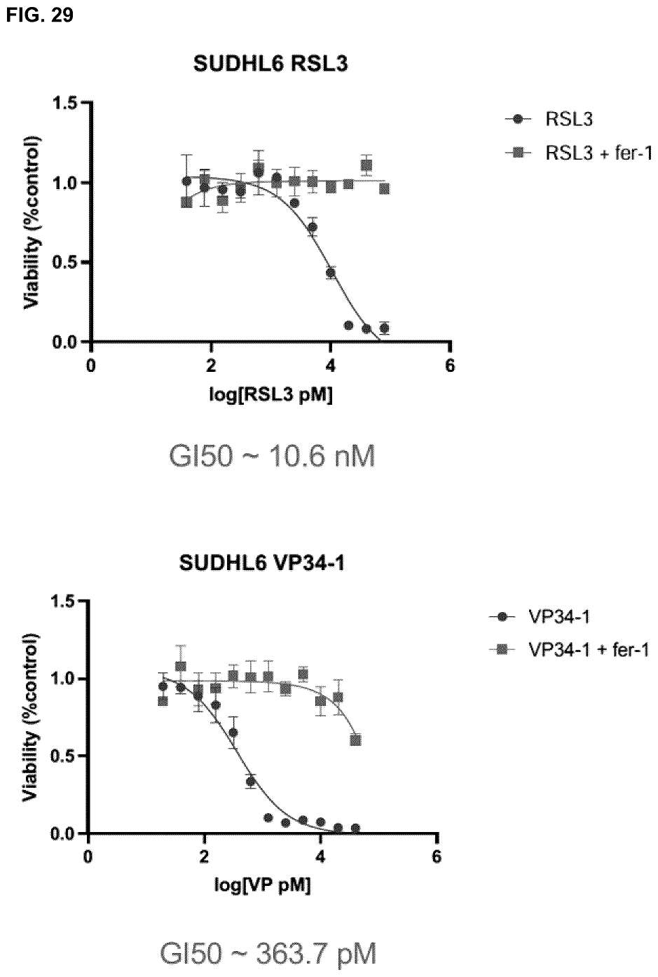

Specifically, we found that introducing a —Br at 5-position of the indole (compound VP-34) led to higher potency and selectivity. As shown in FIG. 28, a less electronegative halogen at the 6-position showed higher potency (comparing VP72-1, VP73-1, VP21-1), EWG at the 6 position was preferred over the 8-position (comparing VP21-1 and VP69-1), while (1S, 3R) stereochemistry had higher potency (comparing VP21-1 and VP34-1). VP34-1 induces ferroptosis in SU-DHL-6 B cell lymphoma cells with 30-fold greater potency than RSL3 (FIG. 29). Docking of VP34-1 onto double mutant GPX4 is shown in FIG. 30, indicating three functional groups of the compound can be optimized to create highly potent inhibitors.

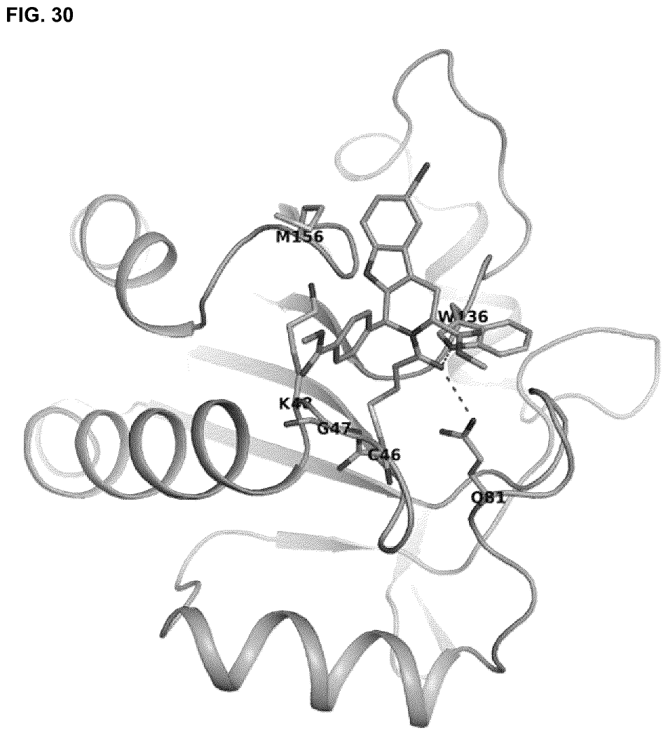

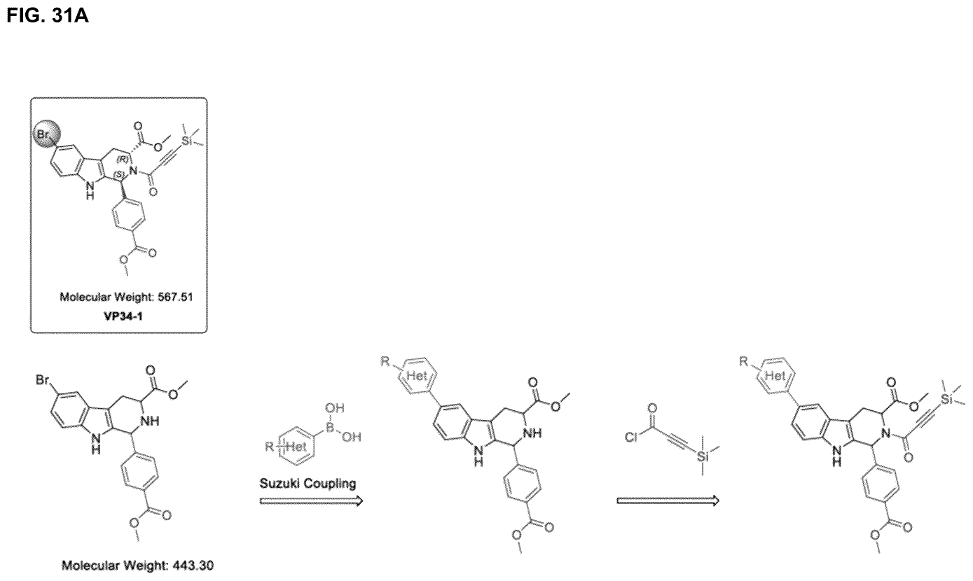

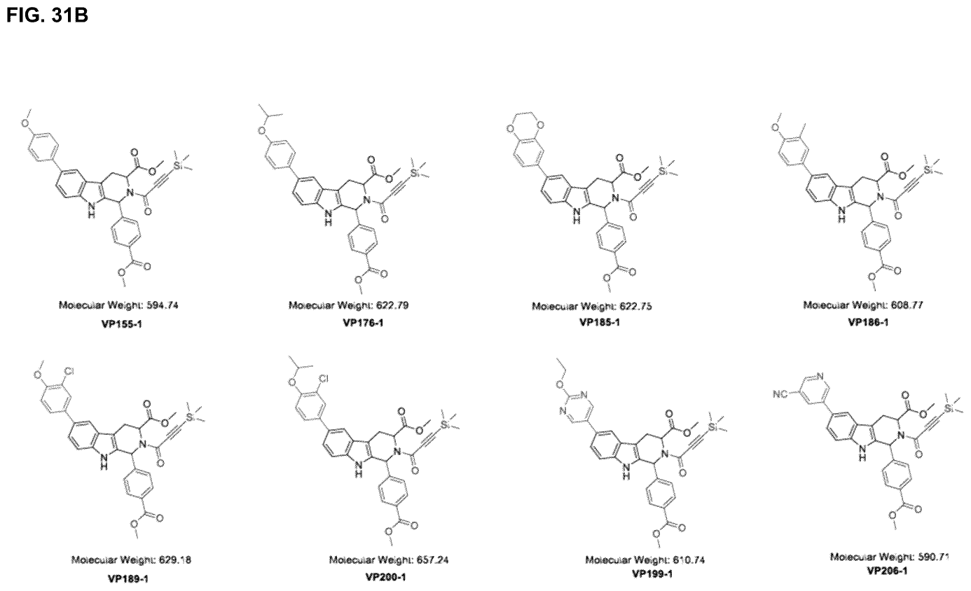

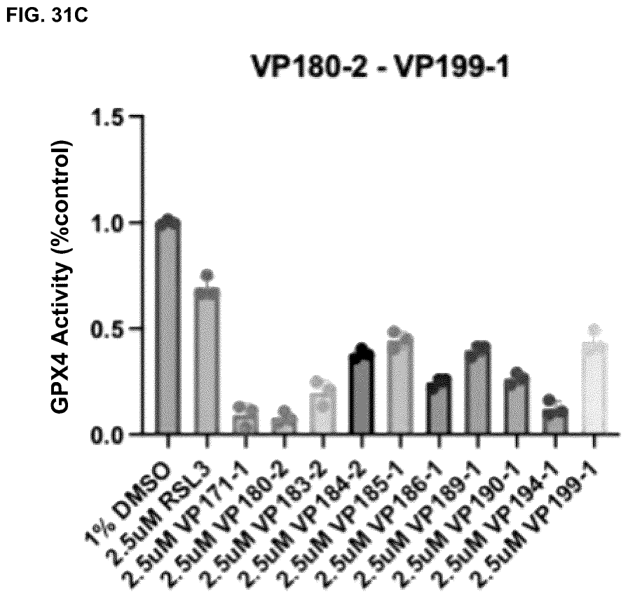

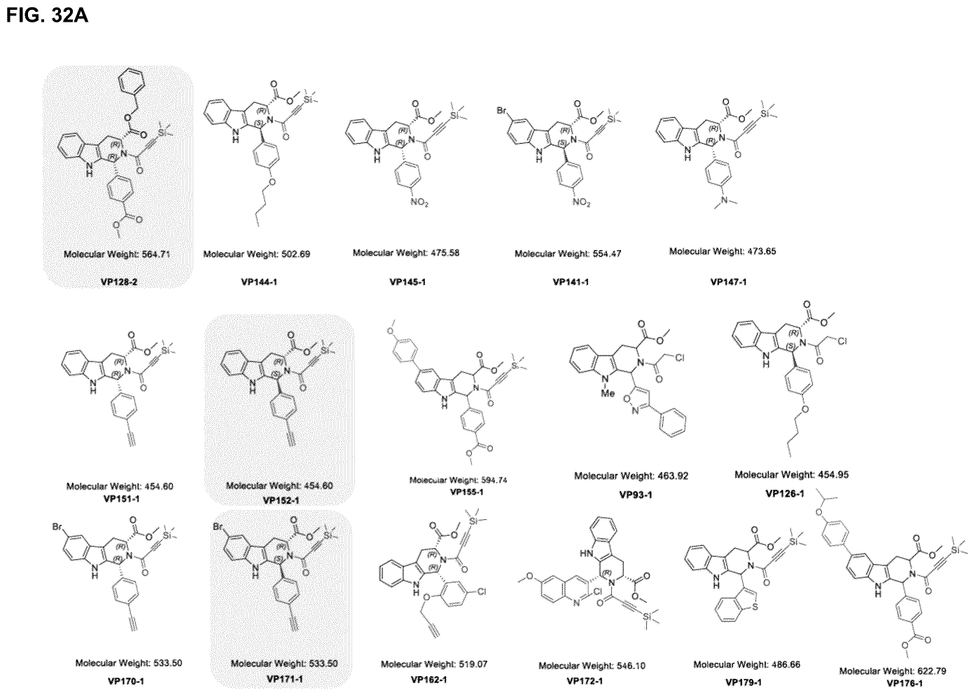

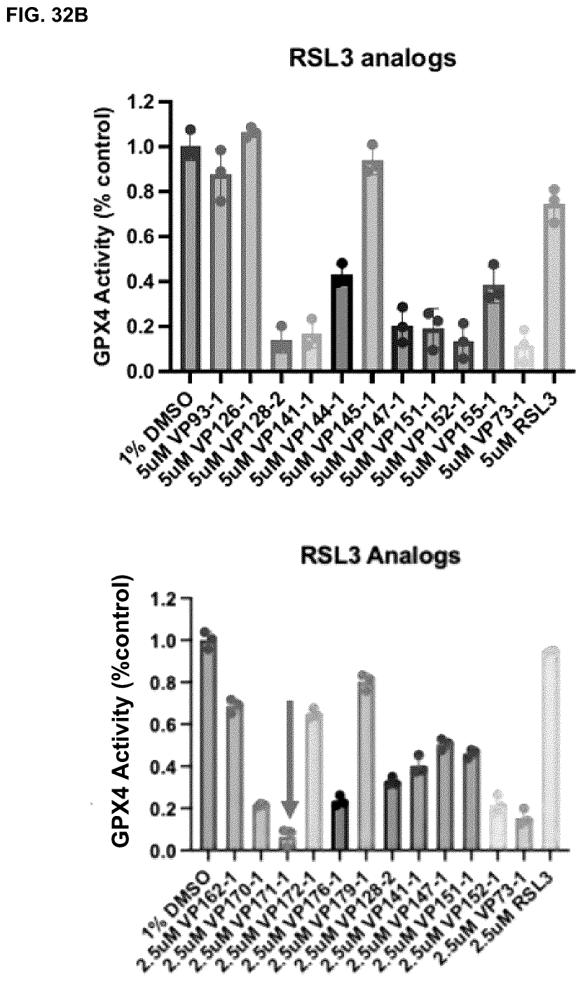

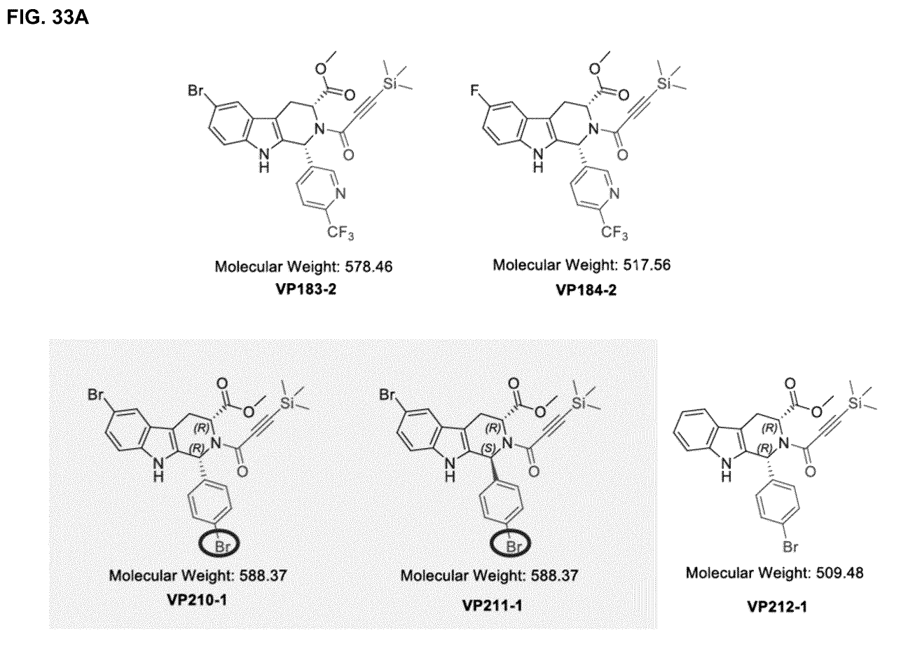

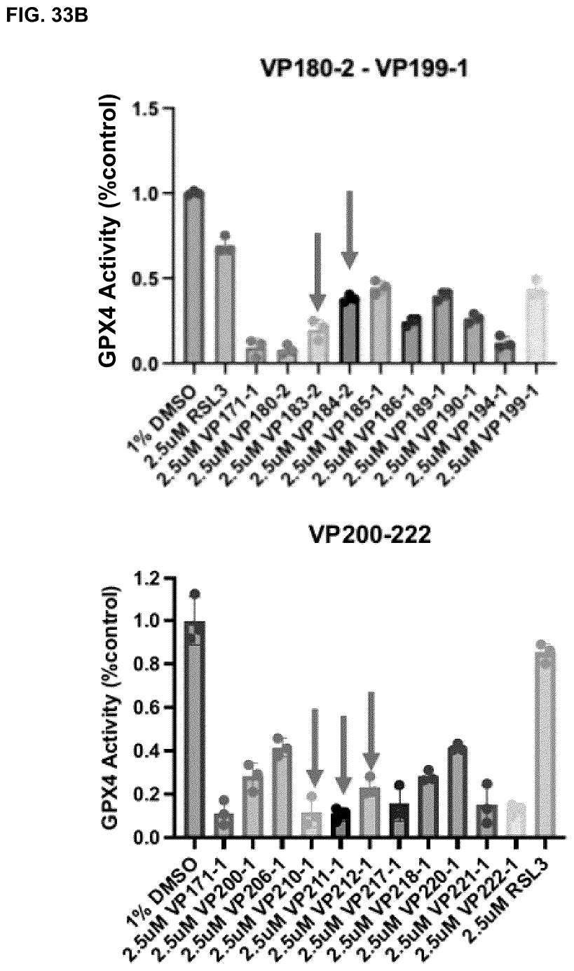

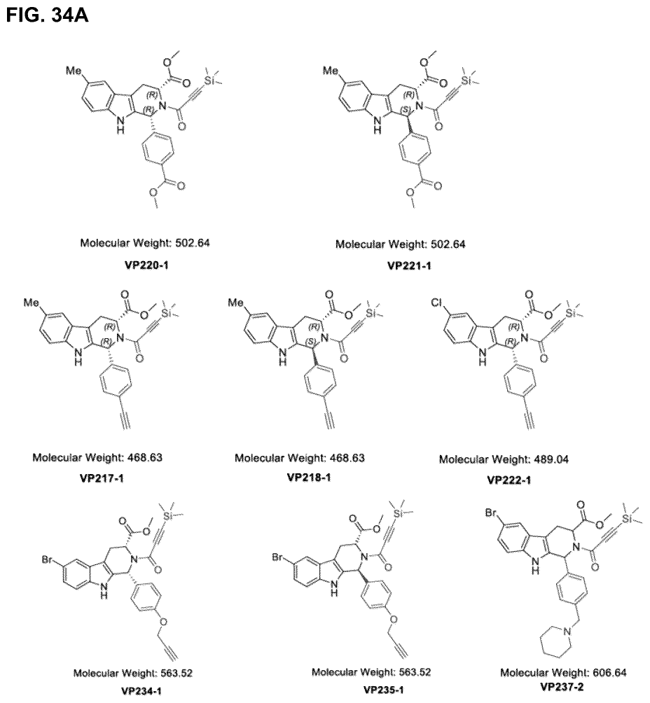

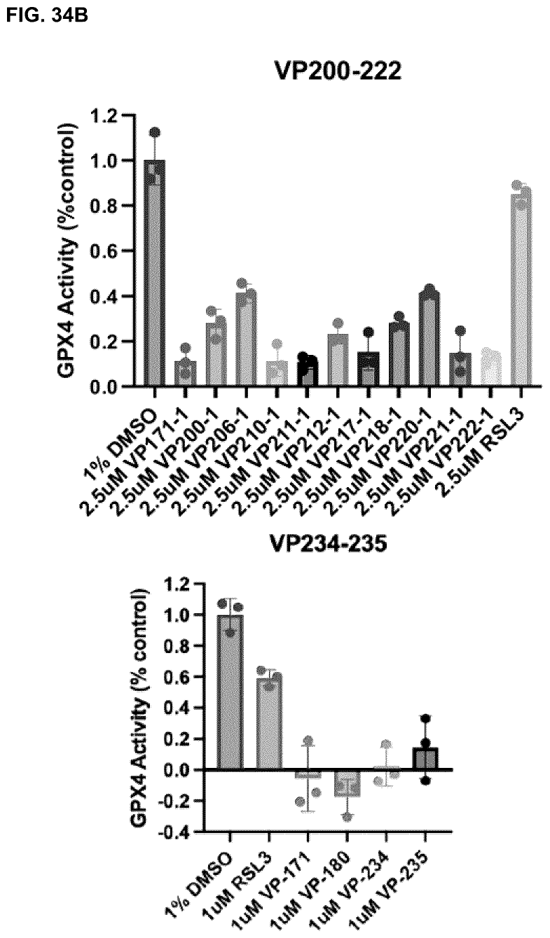

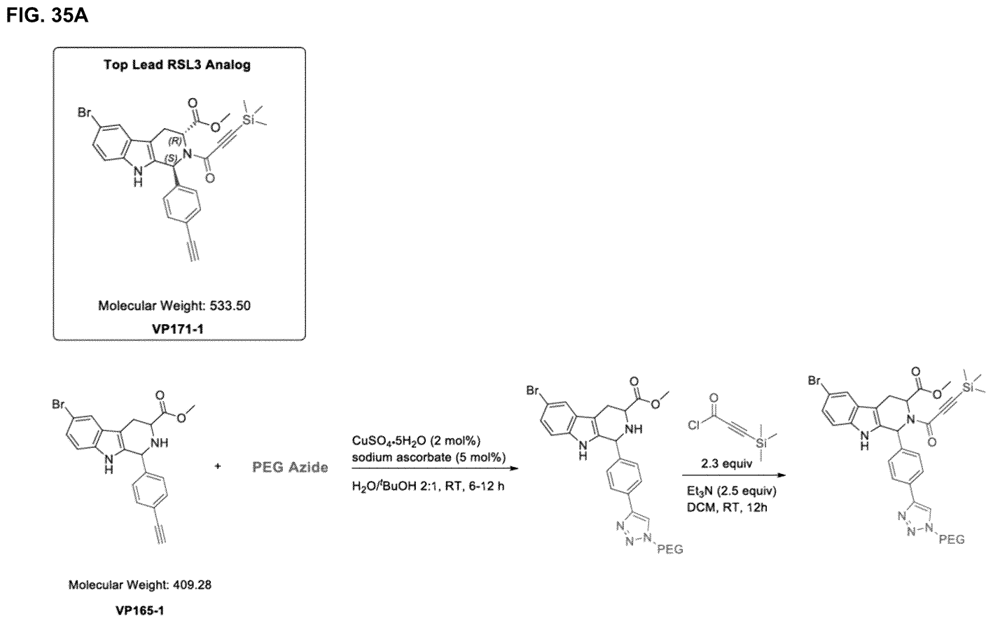

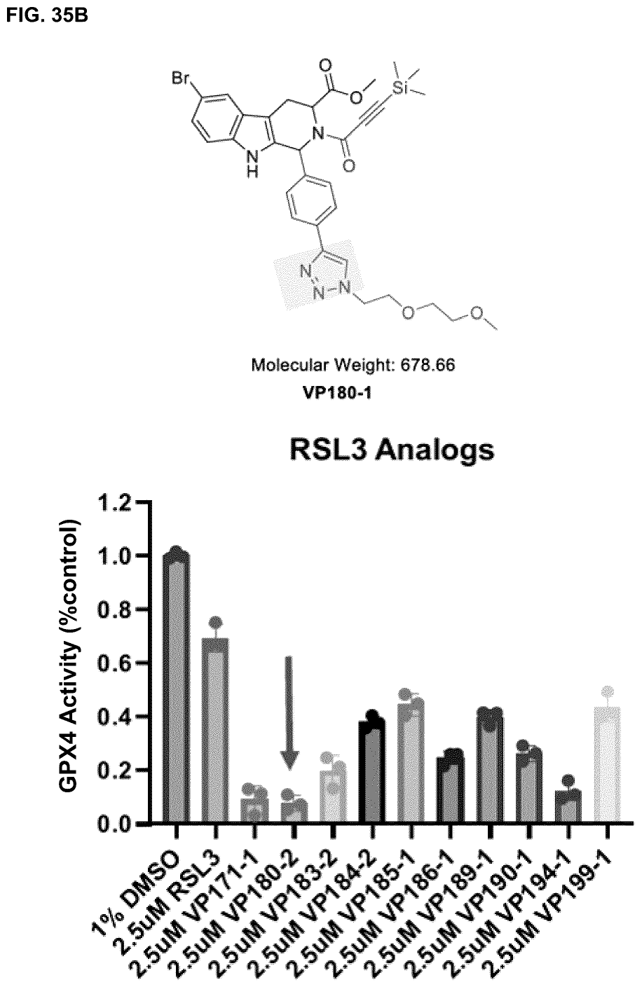

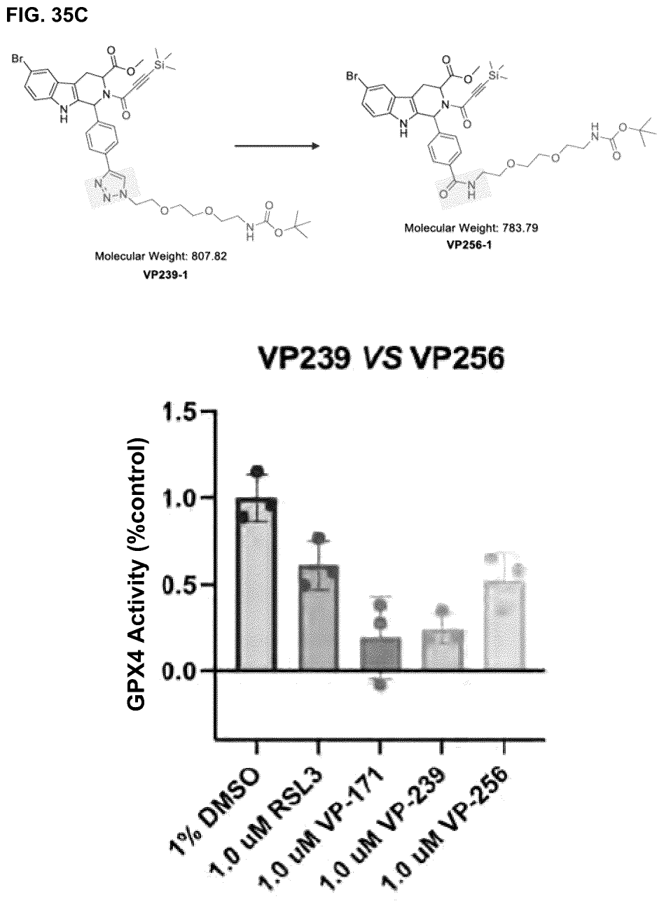

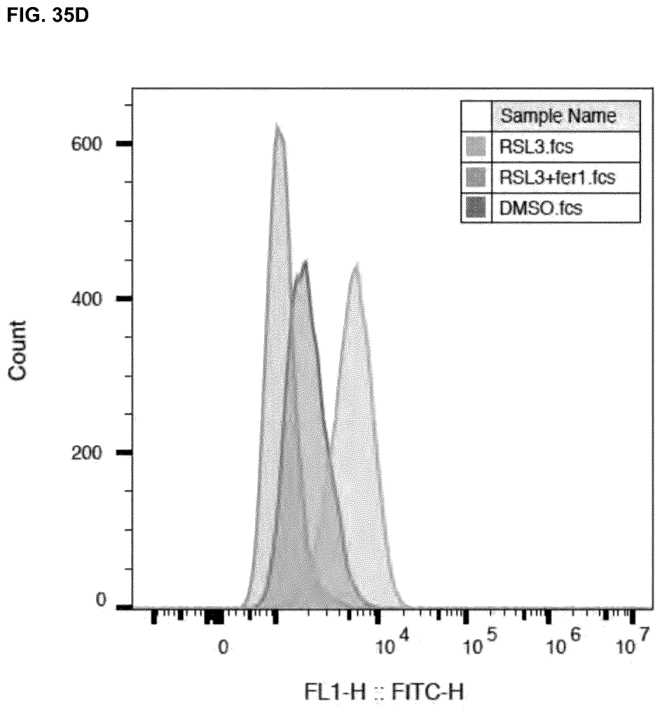

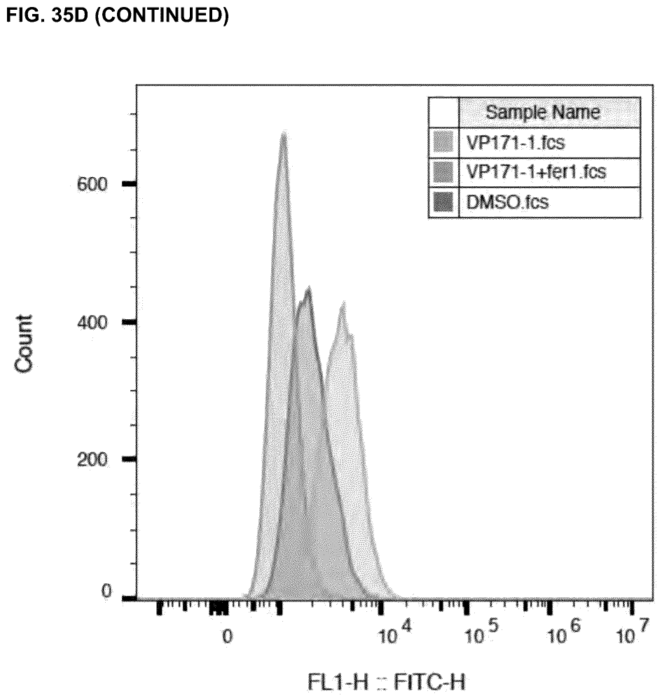

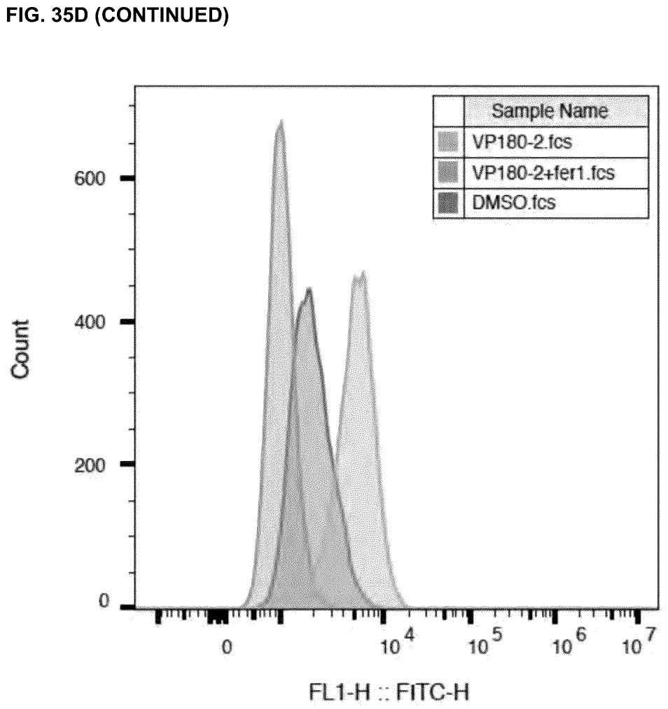

Additionally, we observed that replacement of the —Br group with substituted phenyl rings led to a significant decrease of GPX4 inhibitory activity (FIGS. 31A-31C). Moreover, we performed modifications on the benzoate fragment of RSL3 and we evaluated a series of electron-withdrawing/electron-donating groups and heterocycles, identifying analogs VP-171 (FIGS. 32A-32B) and VP-210 (FIGS. 33A-33B) as early lead compounds. Subsequently, we further modified both analogs in order to increase their solubility and ADMET properties (FIGS. 34A-34B). On the scaffold of compound VP-171, we introduced PEG moieties by employing (CuAAC) click chemistry (FIG. 35A). This series of analogs maintained similar levels of GPX4 inhibitory activity with the parental compound VP-171 (FIG. 35B), while replacing the triazole-moiety with an amide bond resulted in loss of GPX4 inhibitory activity (FIG. 35C). Both VP171 and VP180 induced fer-1 rescuable lipid peroxidation (FIG. 35D).

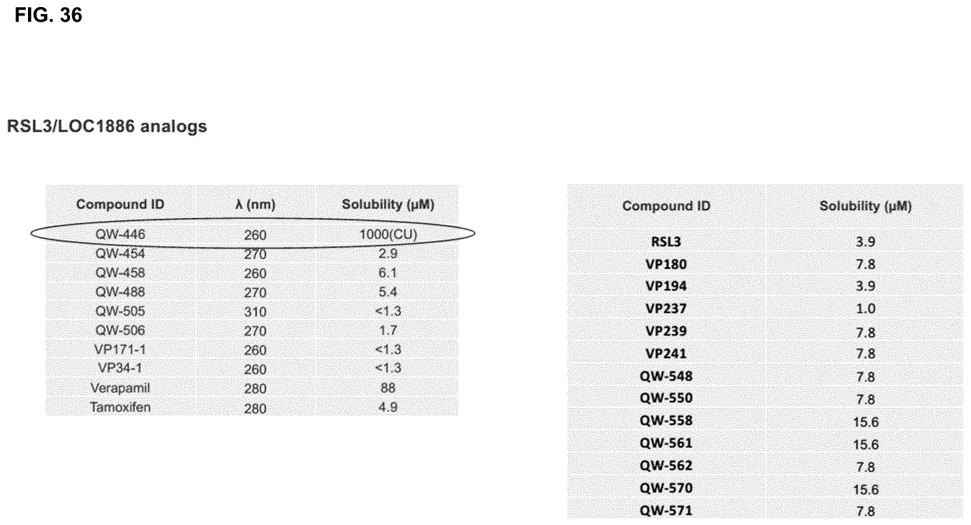

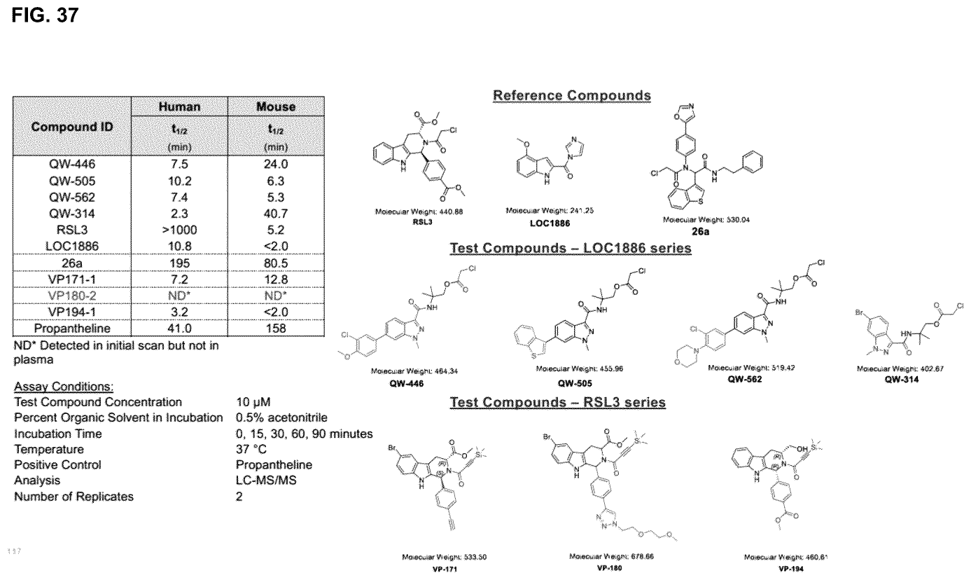

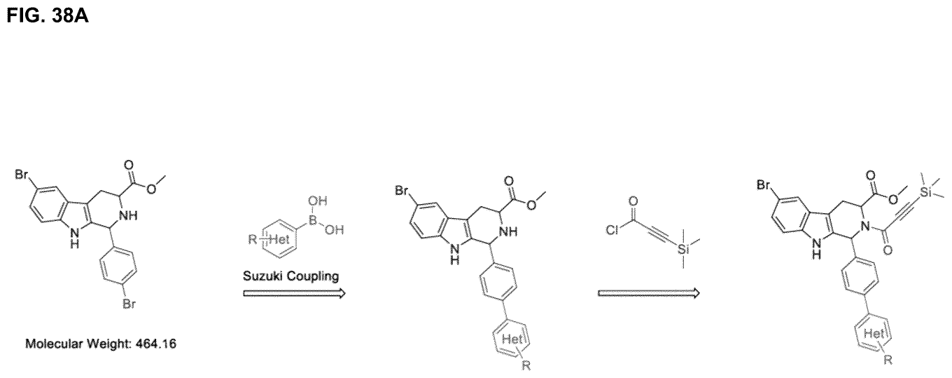

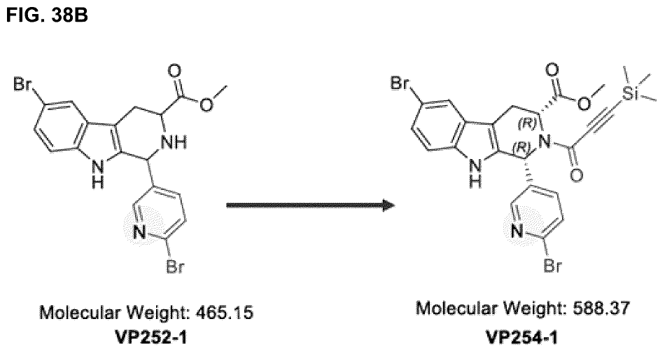

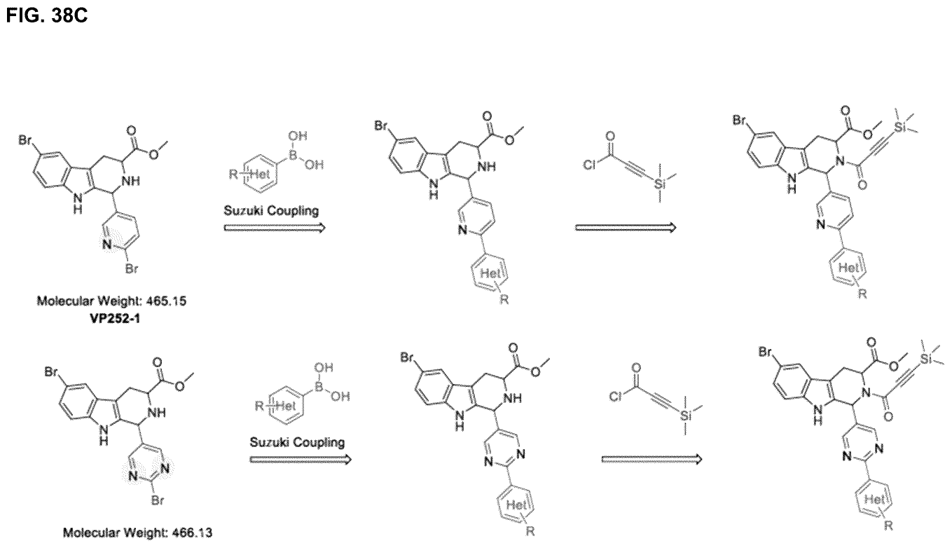

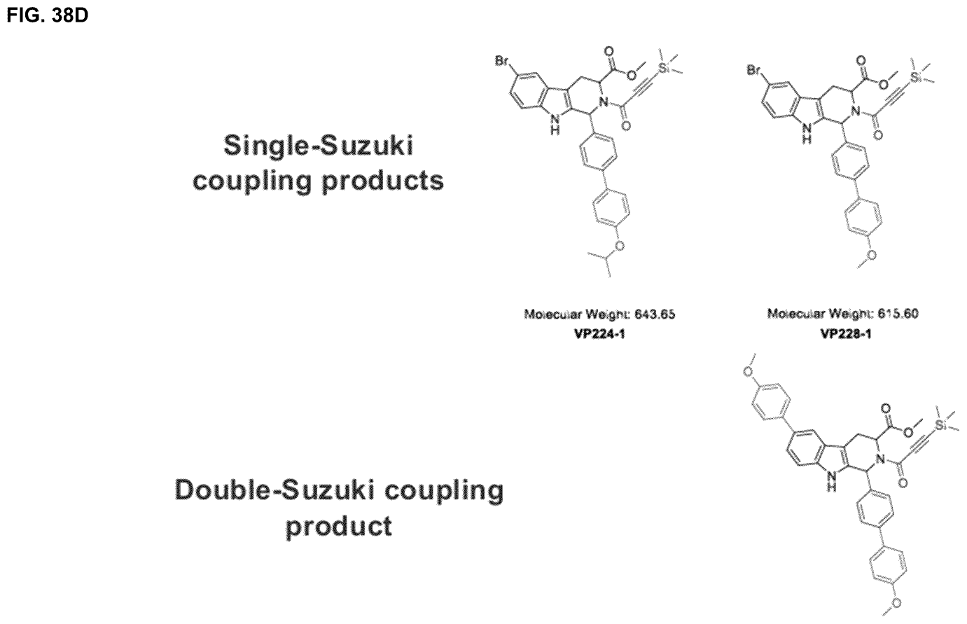

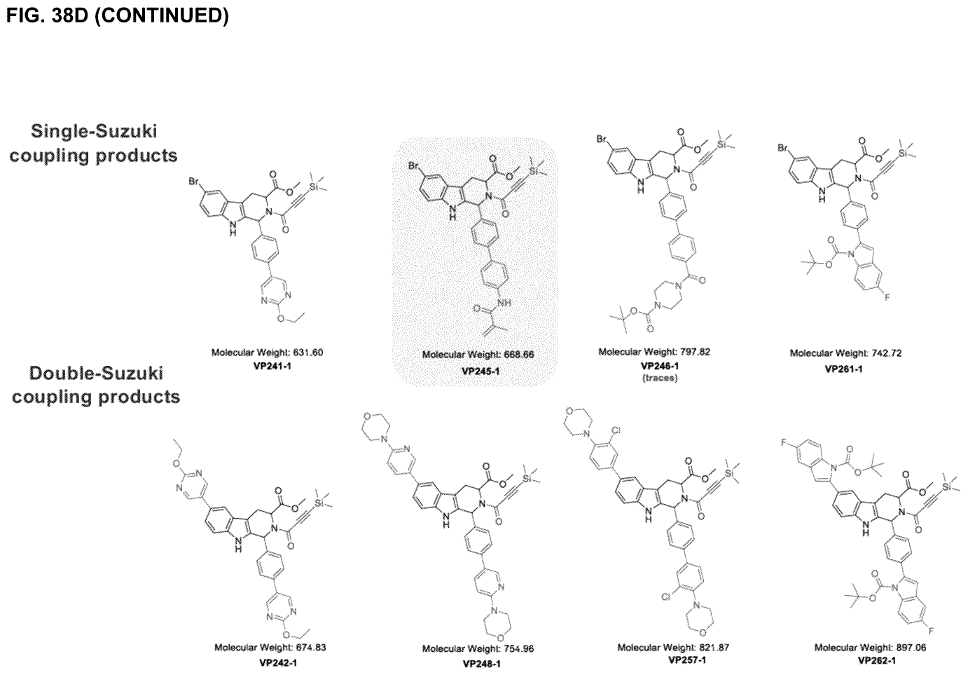

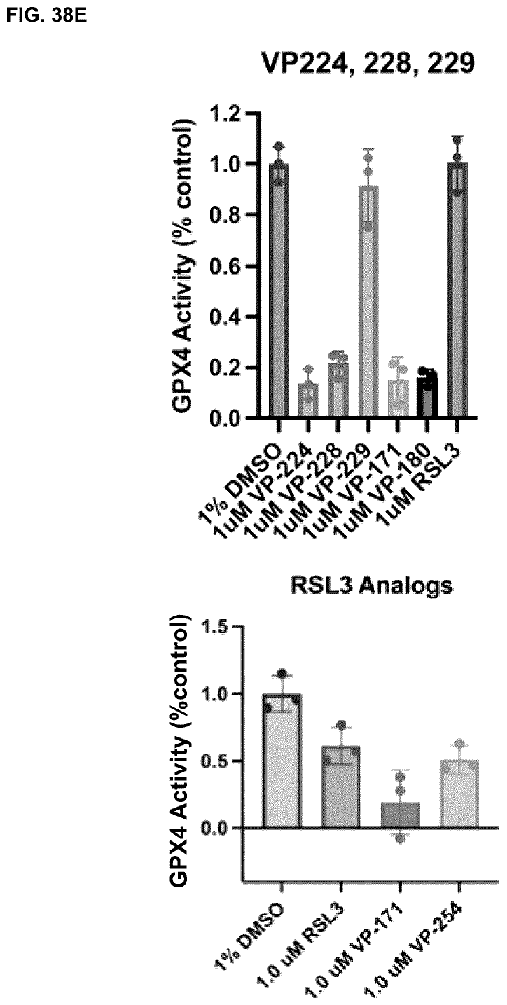

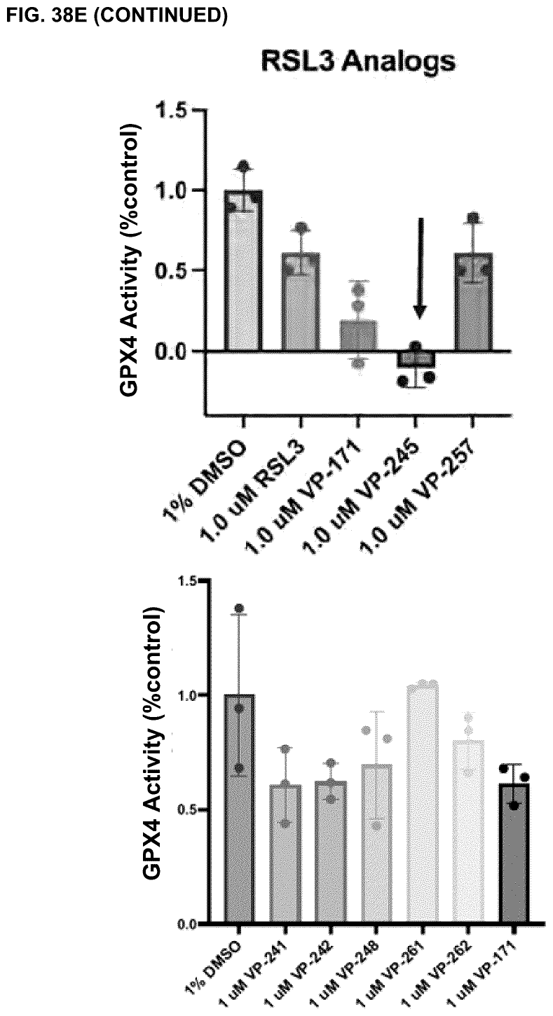

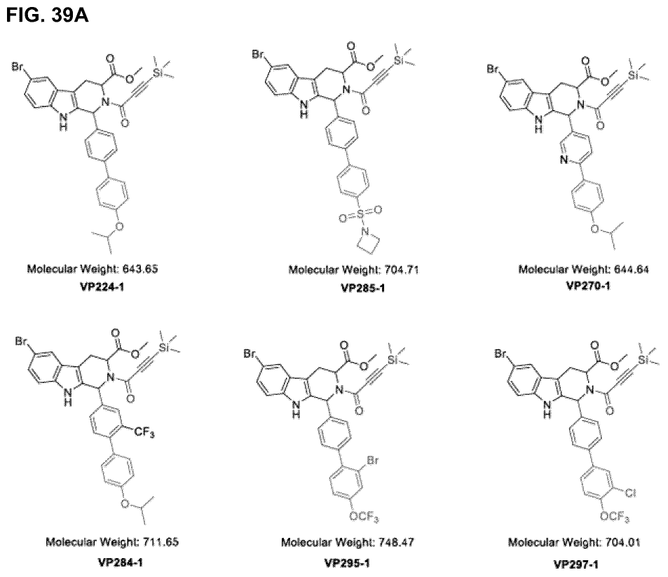

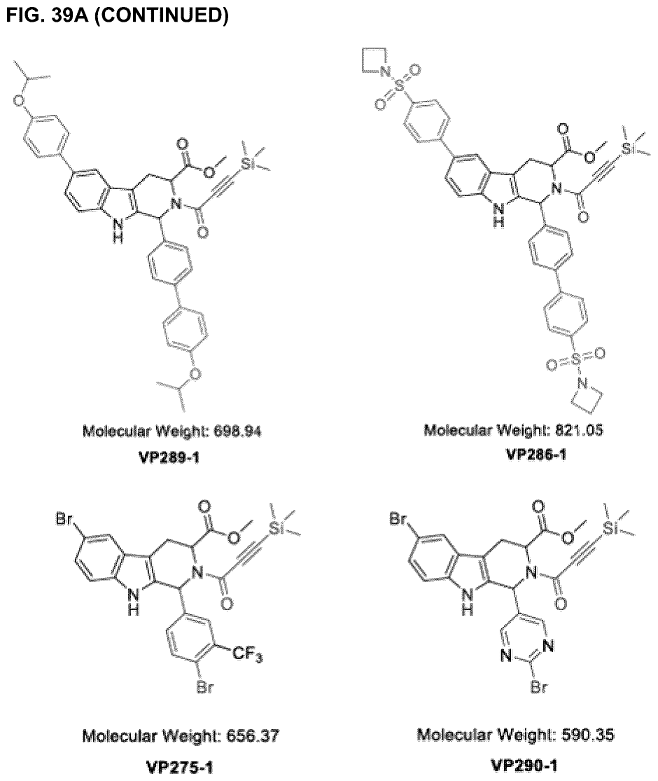

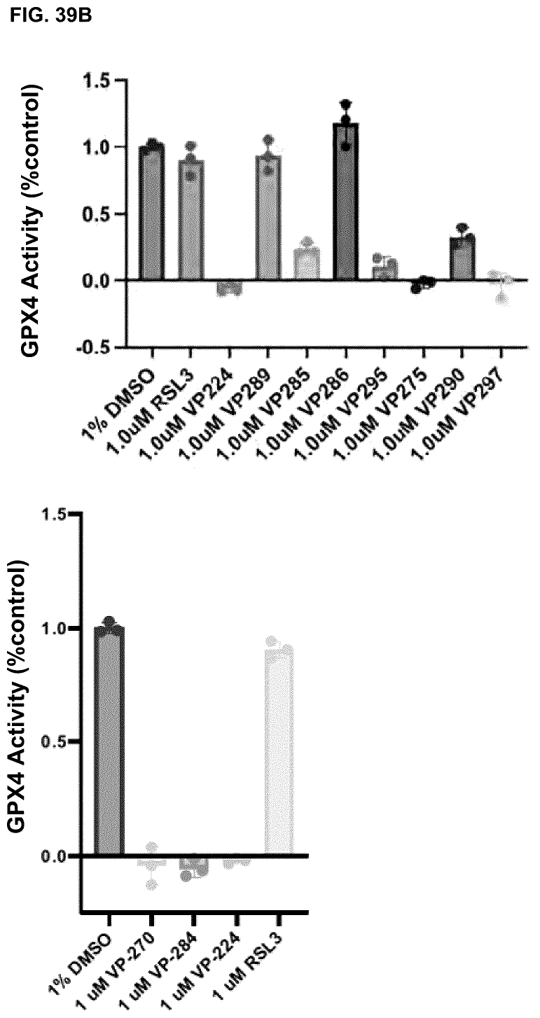

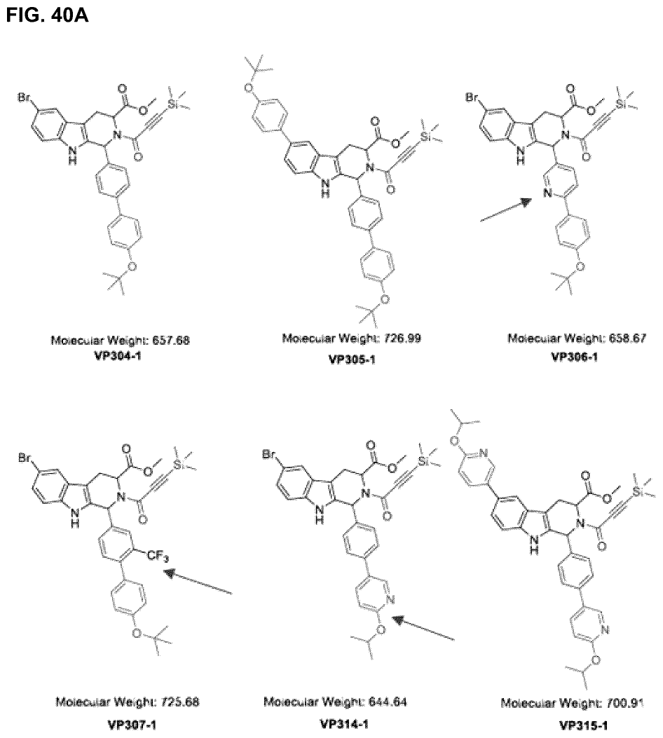

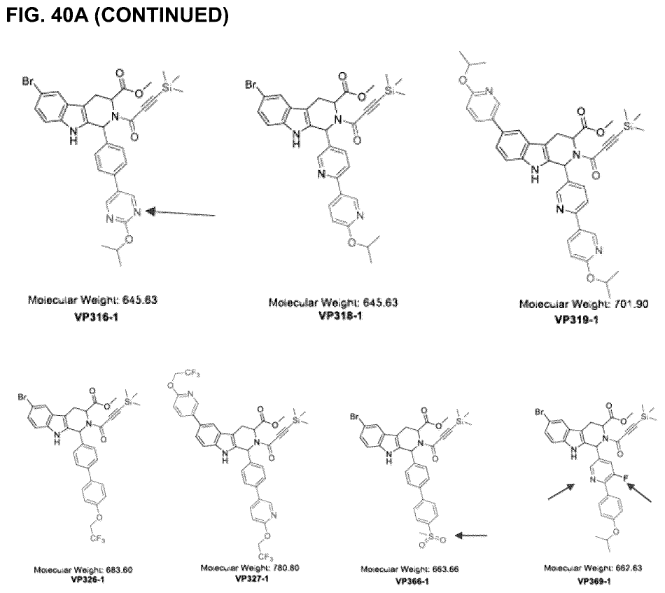

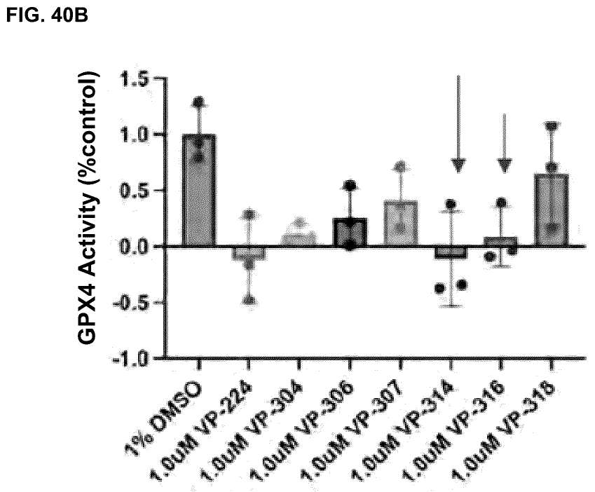

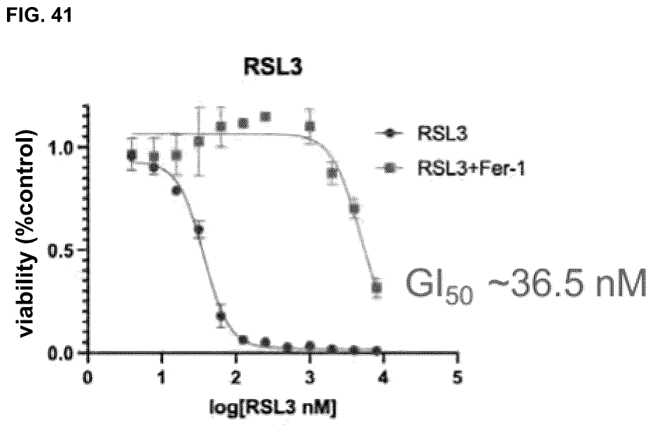

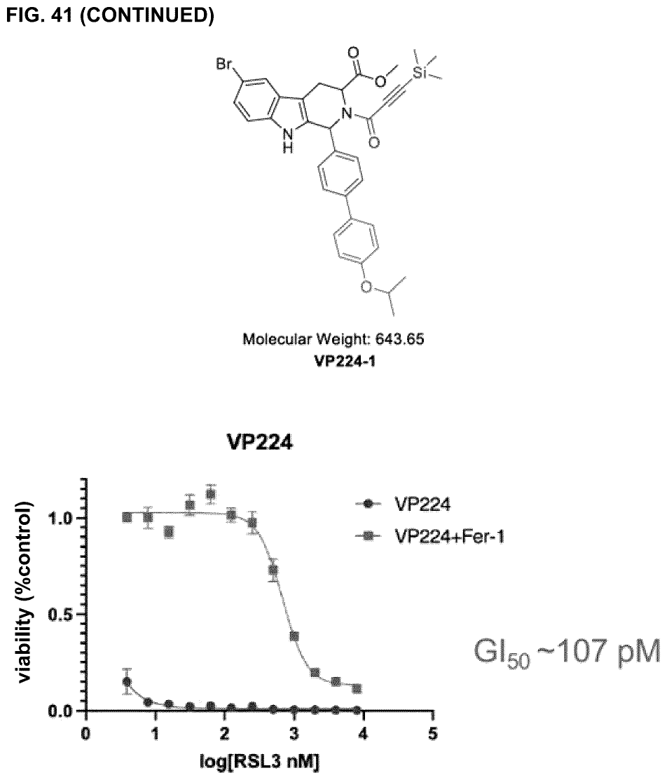

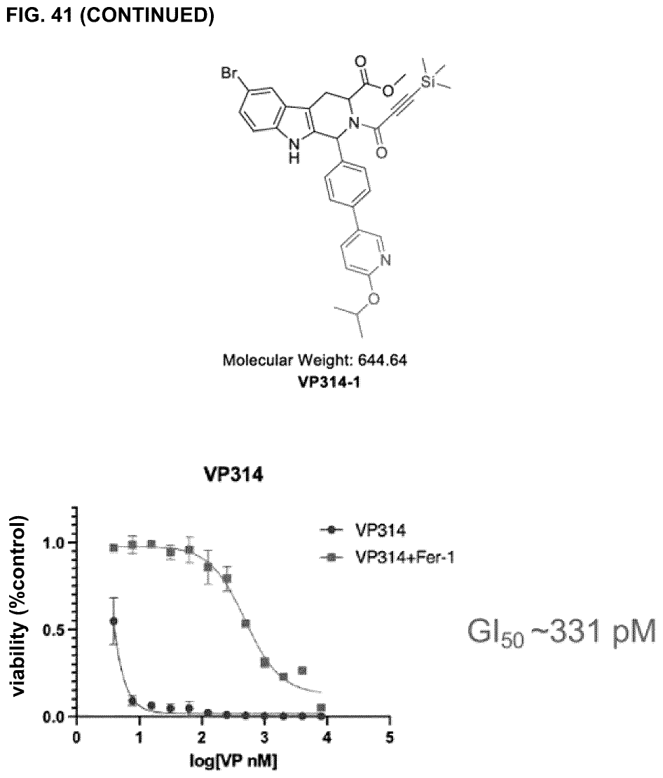

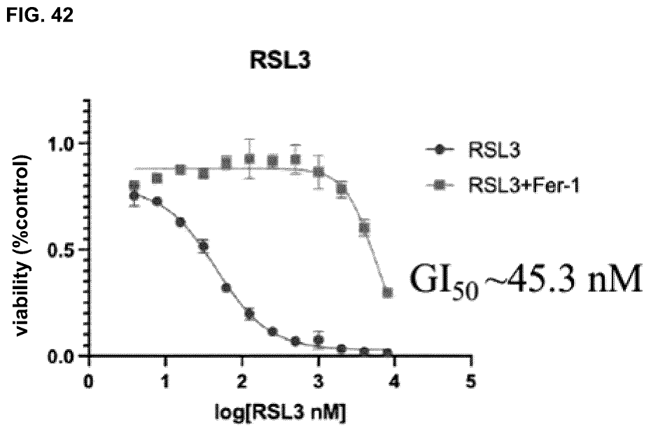

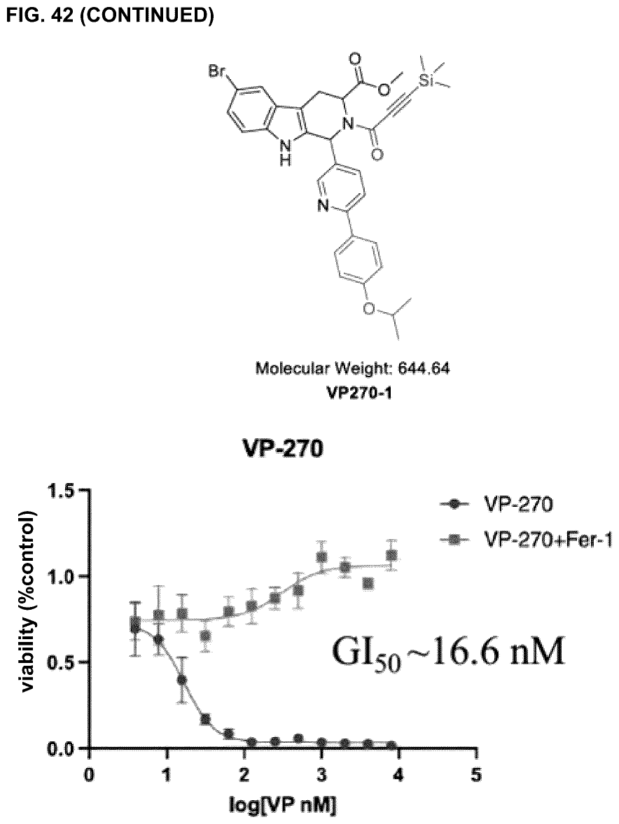

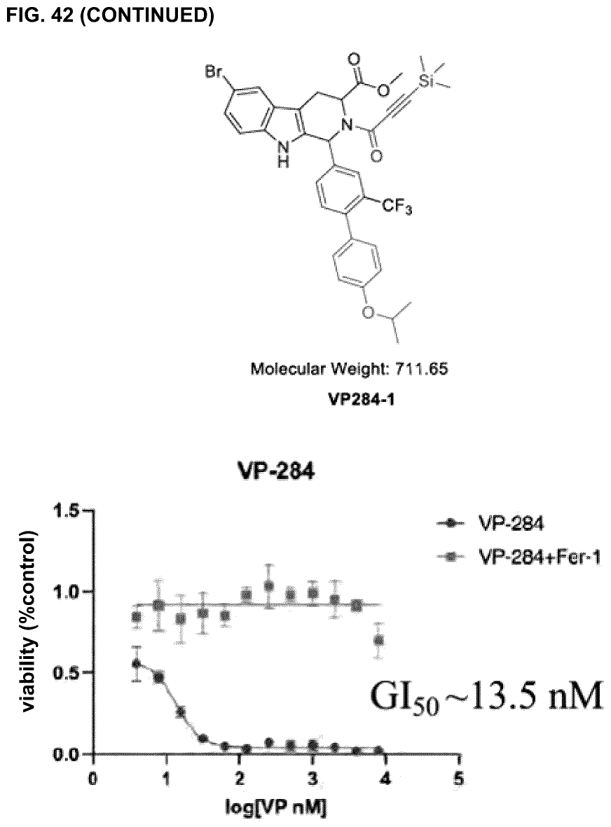

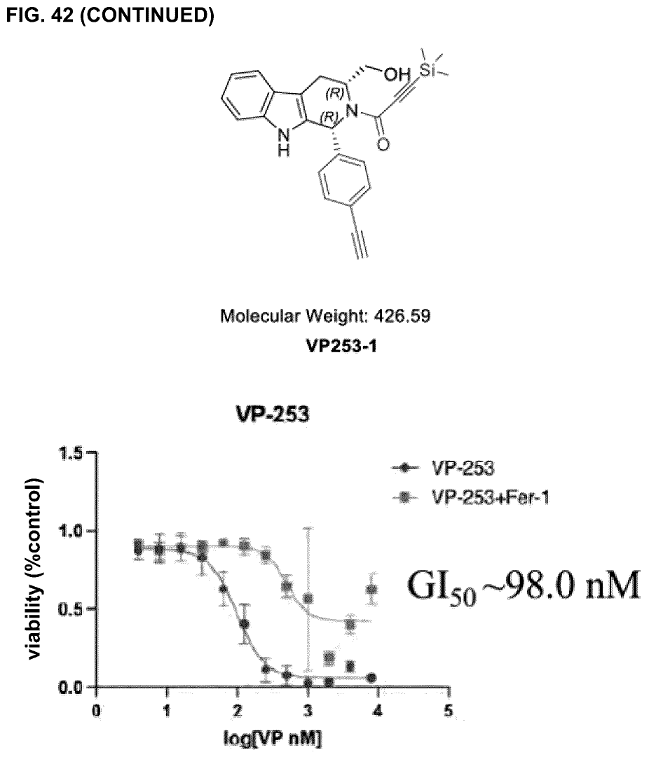

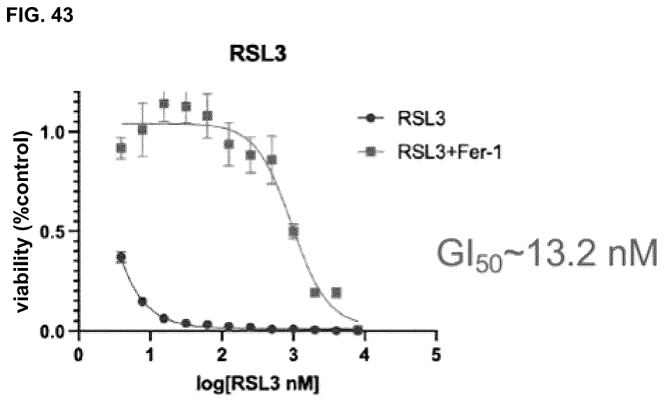

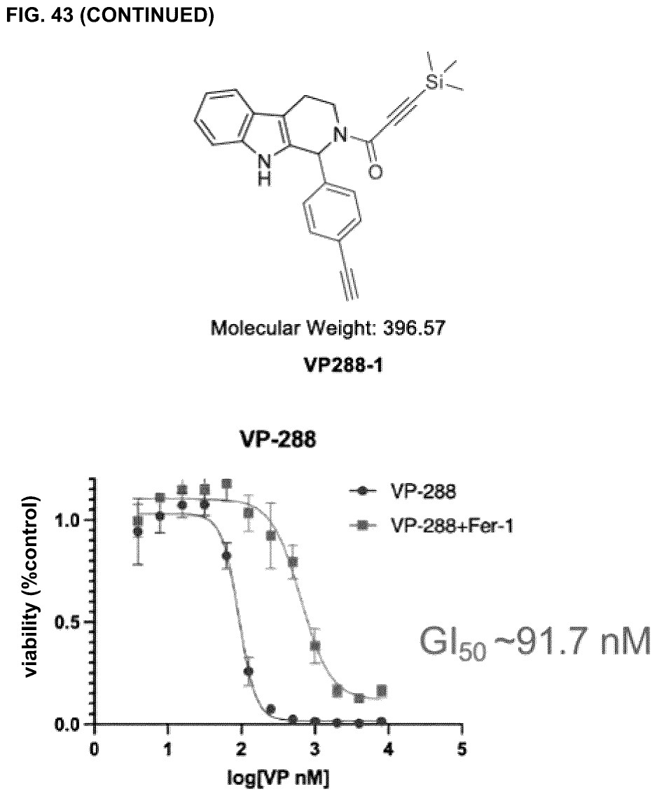

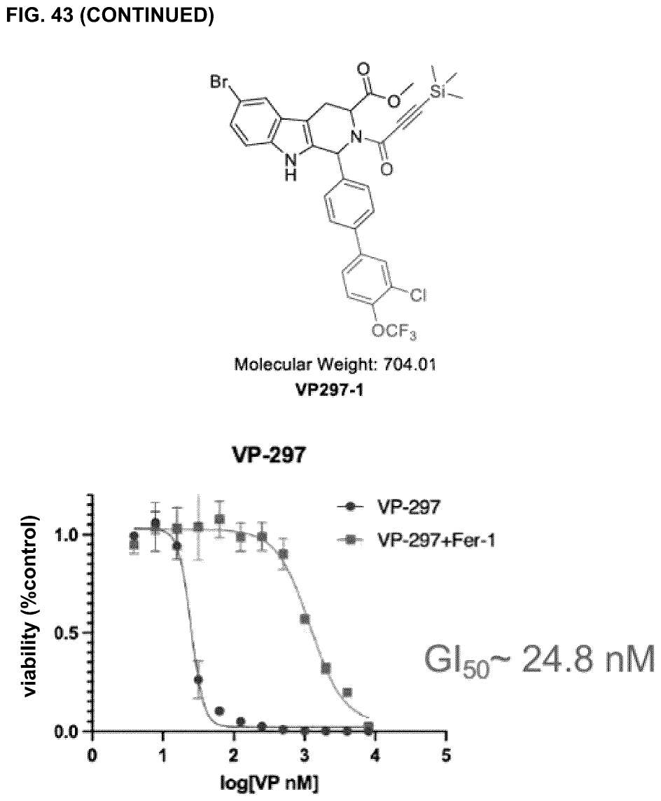

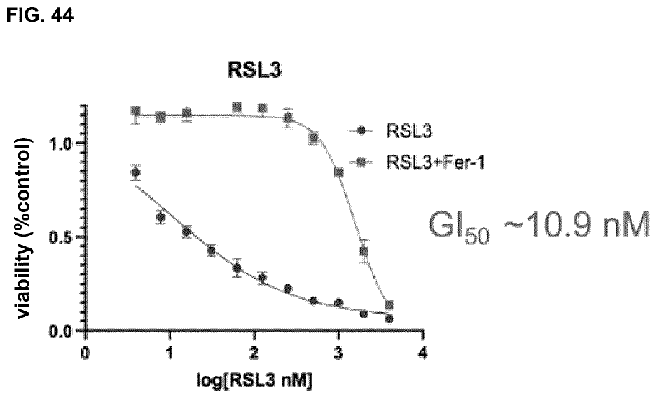

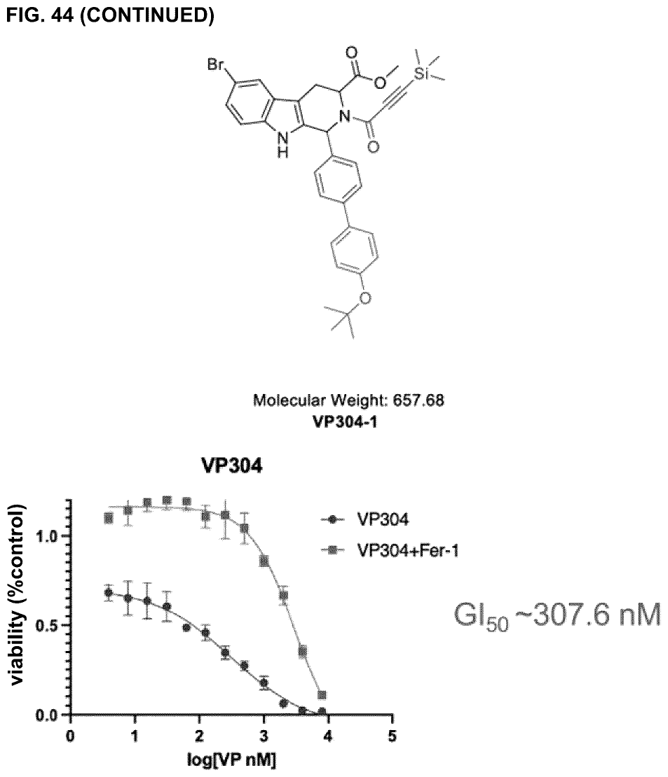

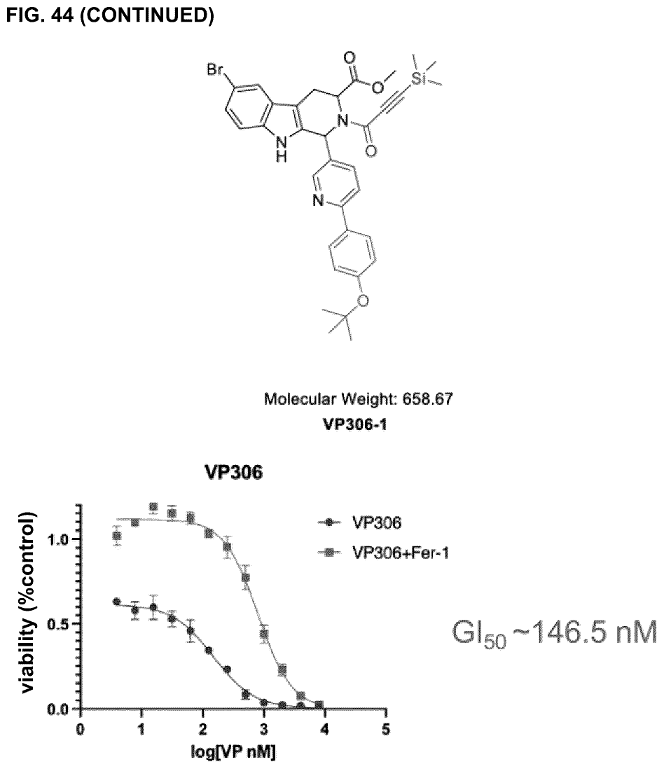

However, the introduction of PEG groups did not improve the compounds' solubility (FIG. 36) and plasma stability (FIG. 37). For compound VP-210, we introduced a substituted phenyl ring or aza-arene via Suzuki coupling while keeping the —Br on the indole ring intact (FIGS. 38A-38C). This series of analogs (FIG. 38D) showed a significant increase in GPX4 inhibitory activity when compared to the reference compound RSL3 (FIG. 38E) as well as improved ADMET properties. Addition of hetero-atom and —CF₃ group results in similar GPX4 inhibitory activity in vitro (FIGS. 39A-39B), and small modifications on the phenyl rings were mainly well-tolerated (FIGS. 40A-40B). VP-314 induced ferroptosis in HT-1080 cells with great potency comparable to lead compound VP-224 (FIG. 41). In general, VP series compounds induced ferroptosis in HT-1080 cells with greater potency than RSL3 (FIG. 42) except VP-288, VP-297, VP-304 and VP-306 (FIGS. 43-44).

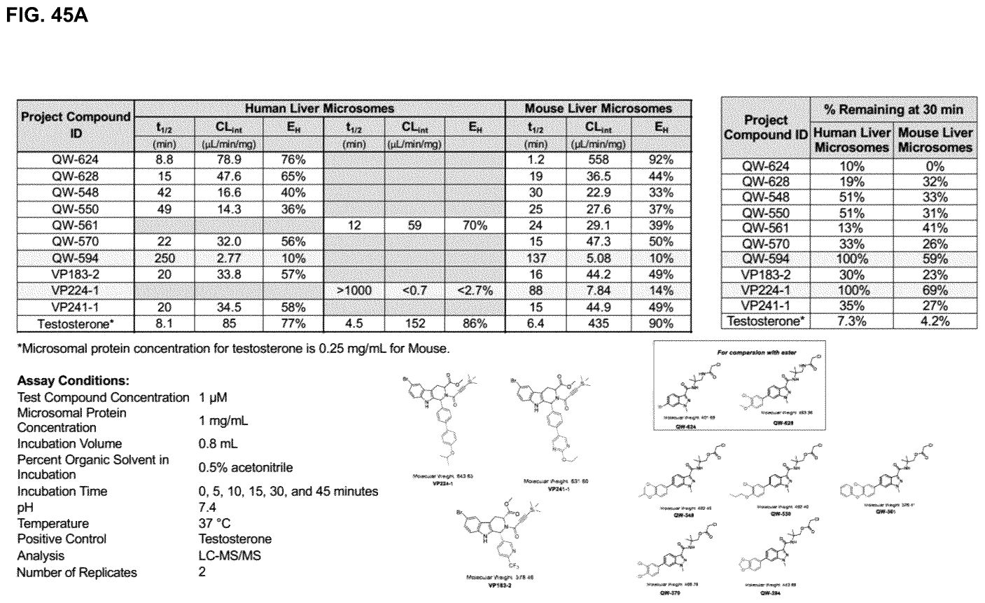

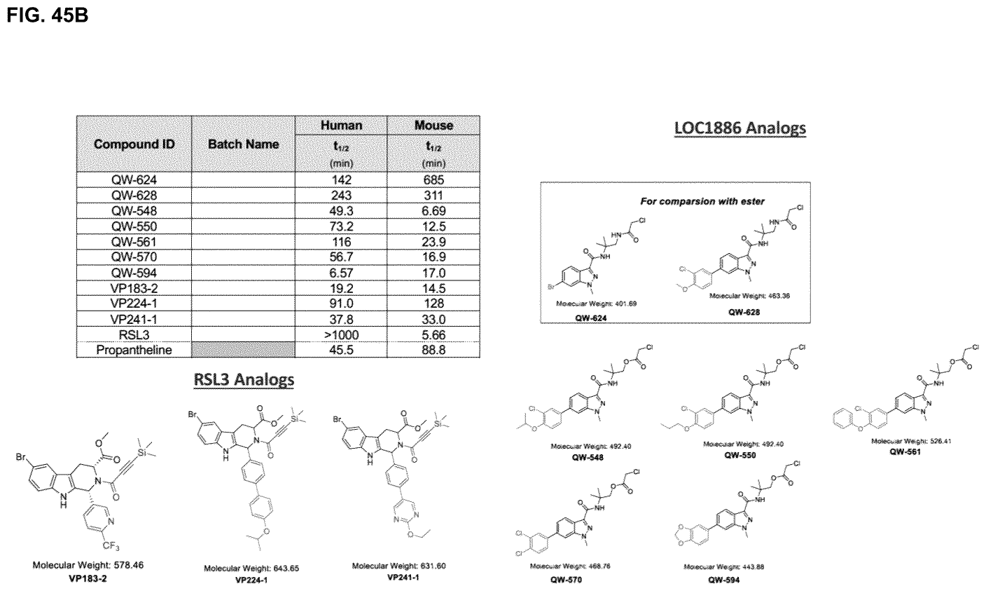

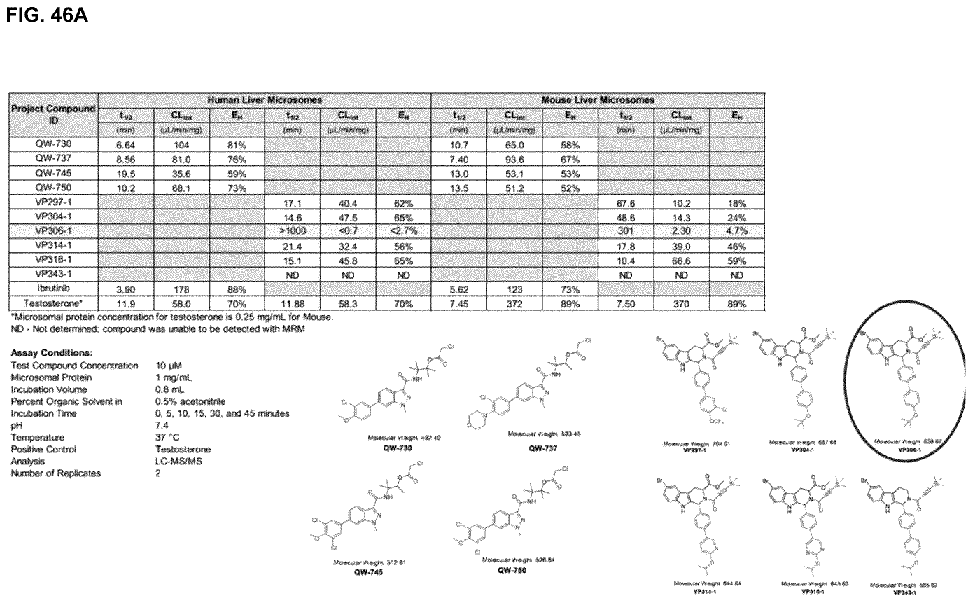

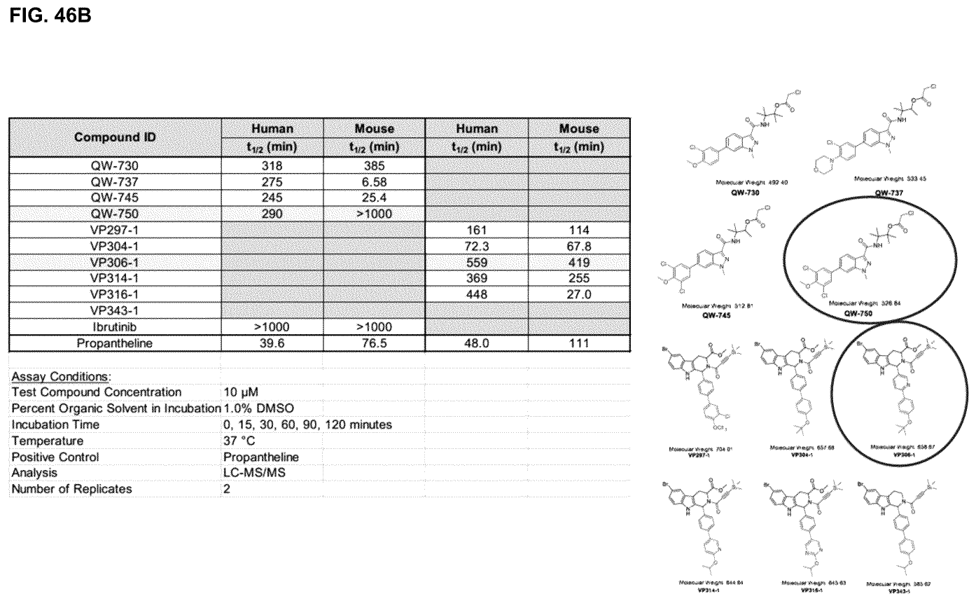

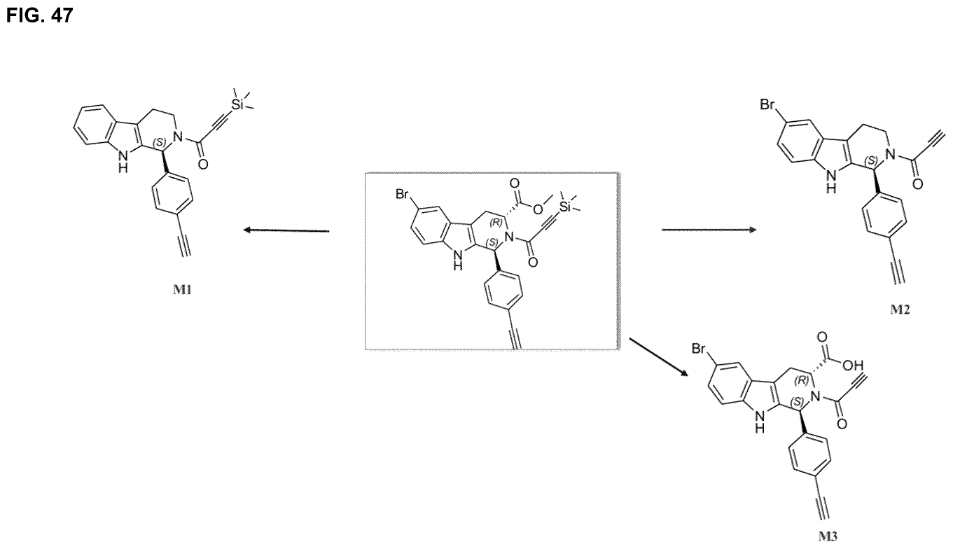

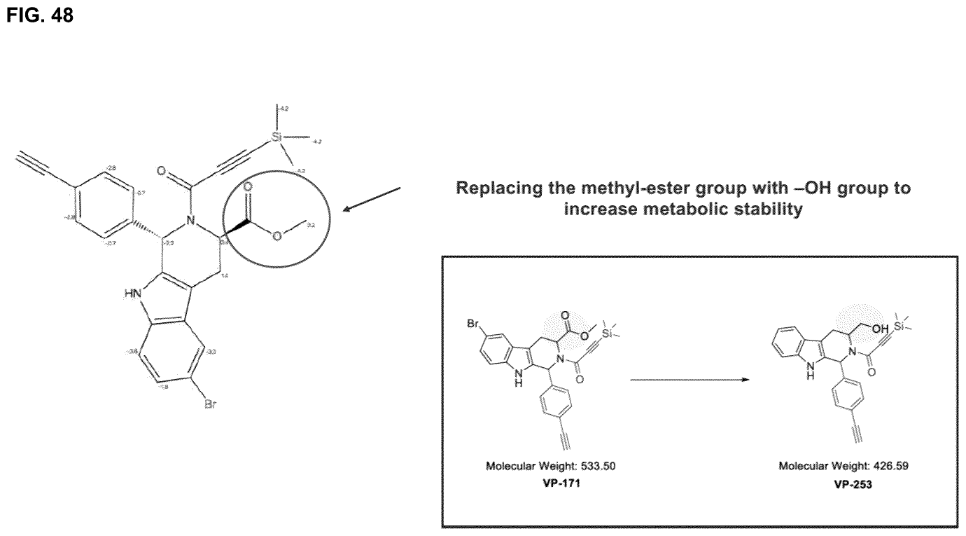

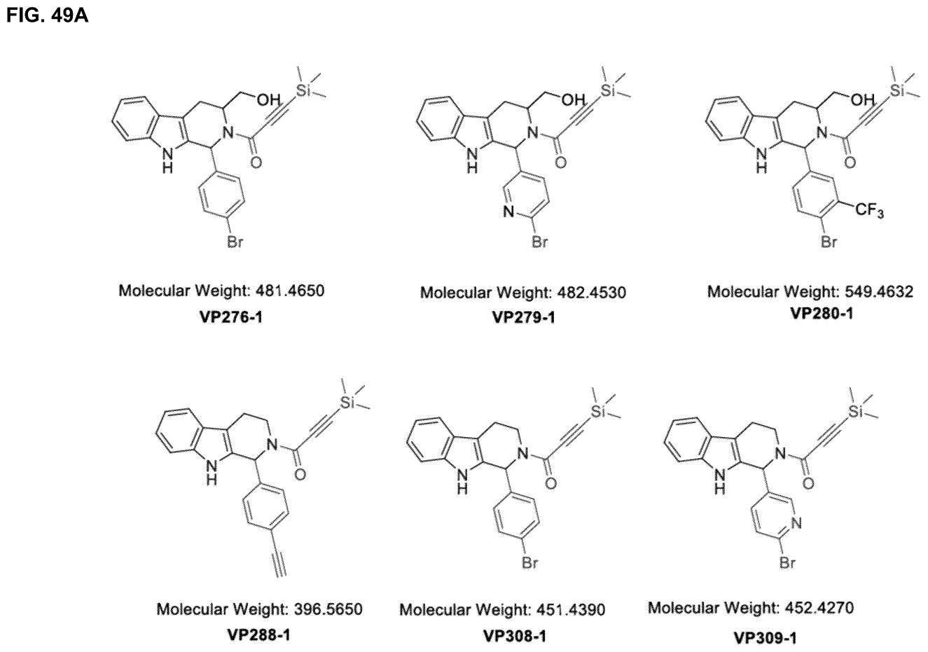

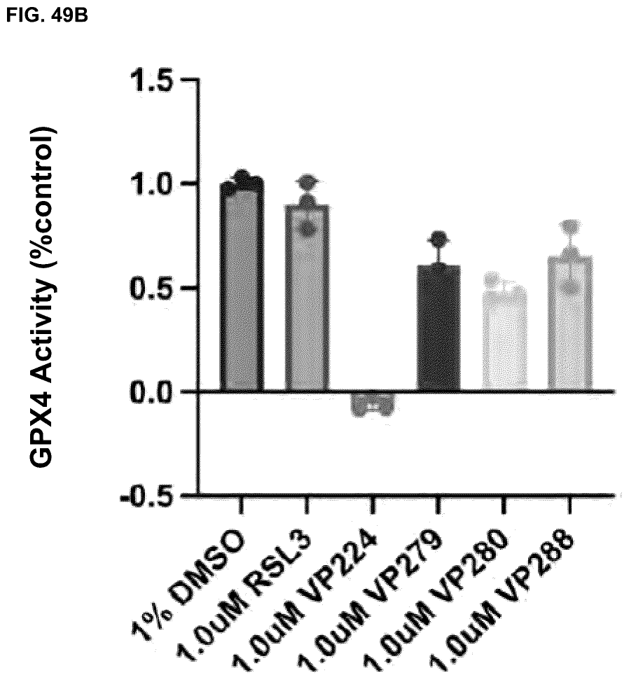

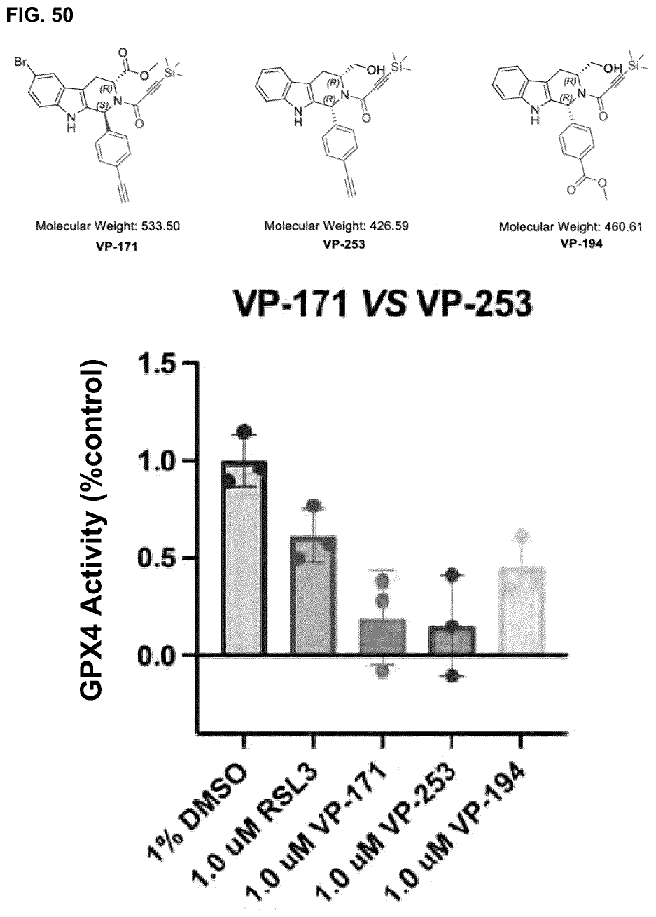

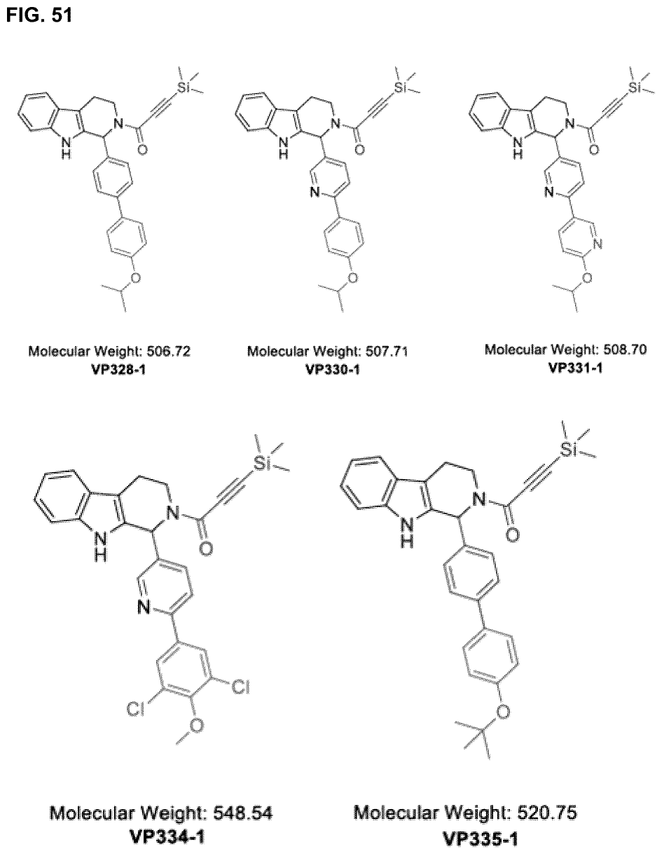

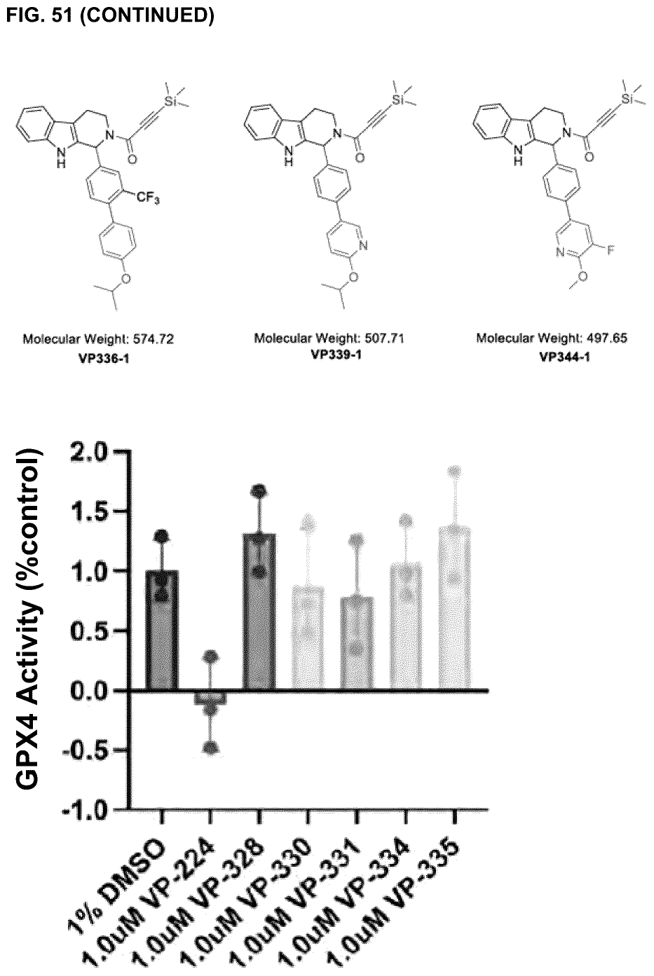

More specifically, among these analogs, we identified our latest top lead compounds VP-224 and VP-306 with significantly improved metabolic and plasma stability (FIGS. 45A-45B and FIGS. 46A-46B). Based on the metabolite identification (MetID) studies that we performed on compound VP-171 (FIG. 47), we found that the methyl ester group constitutes a metabolic liability. In an effort to further increase the stability of our compounds, we synthesized analogs where the methyl ester was either replaced with moieties less susceptible to metabolism or completely removed (FIG. 48 and FIGS. 49A-49B). Replacement of the ester with alcohol on early analogs proved to be tolerated (FIG. 50), while removal of both the ester and bromine led to loss of GPX4 activity (FIG. 51).

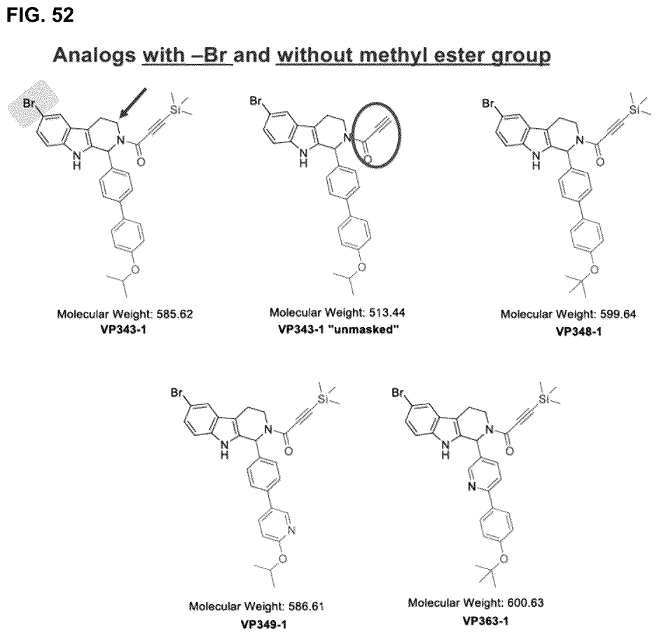

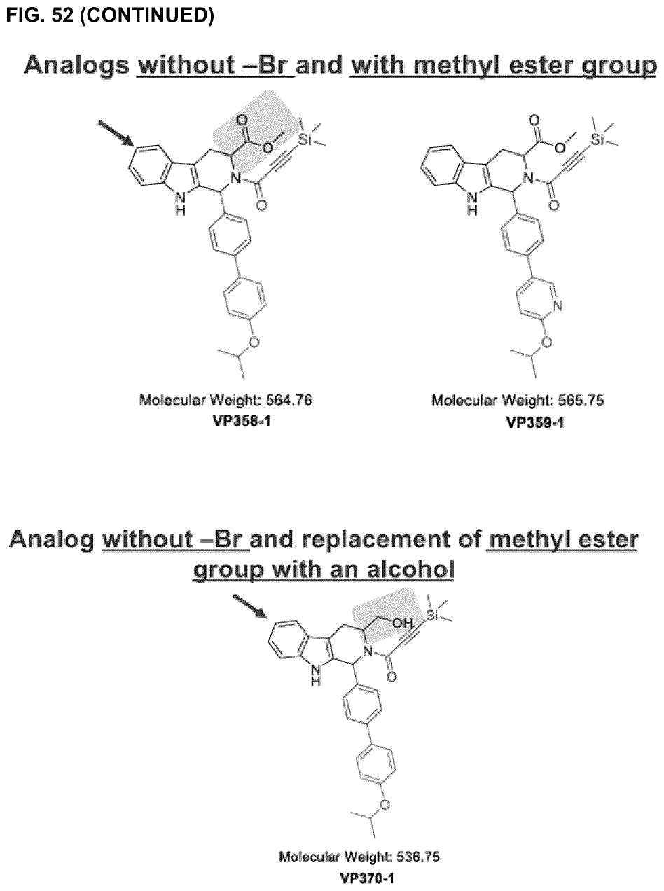

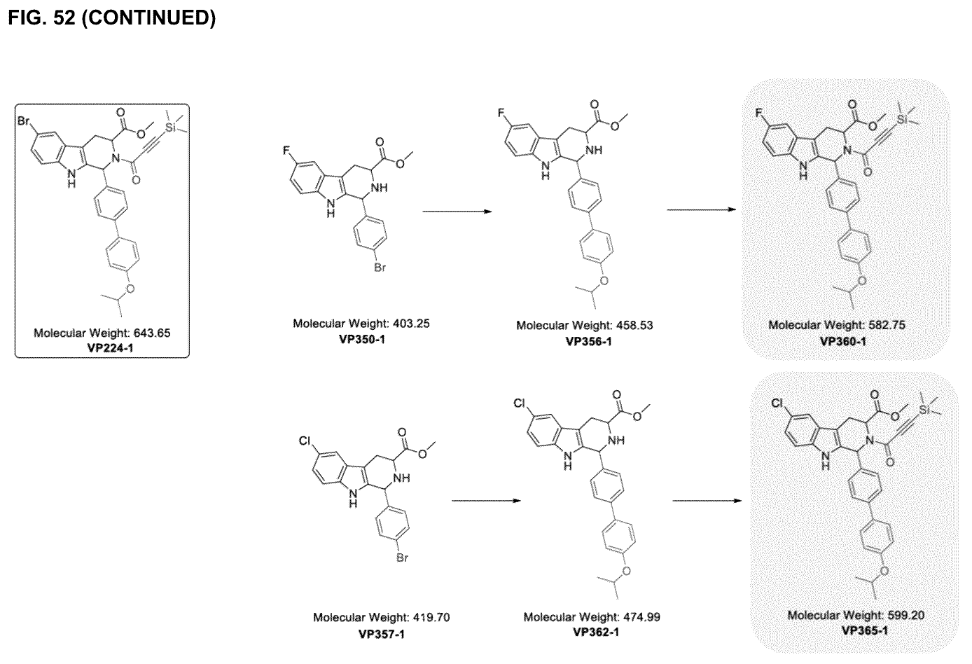

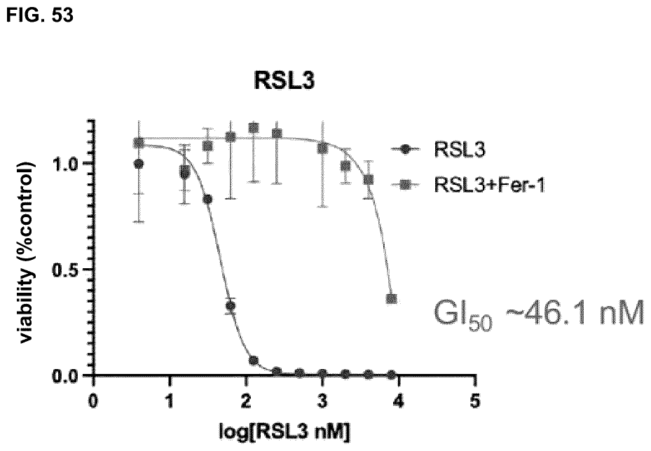

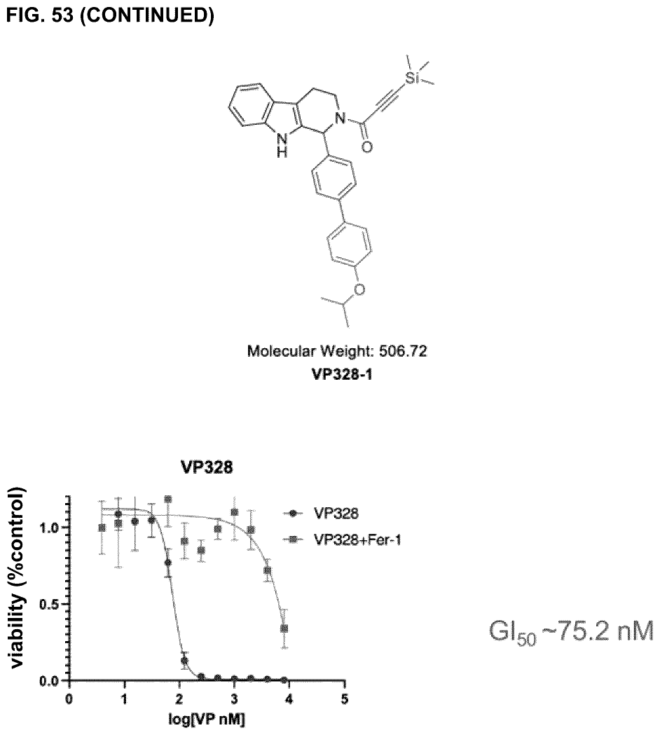

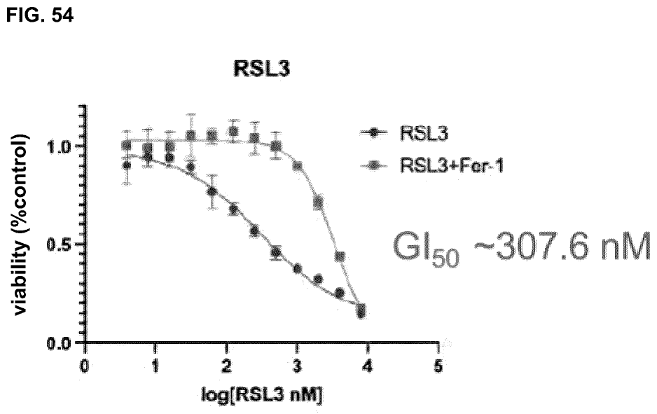

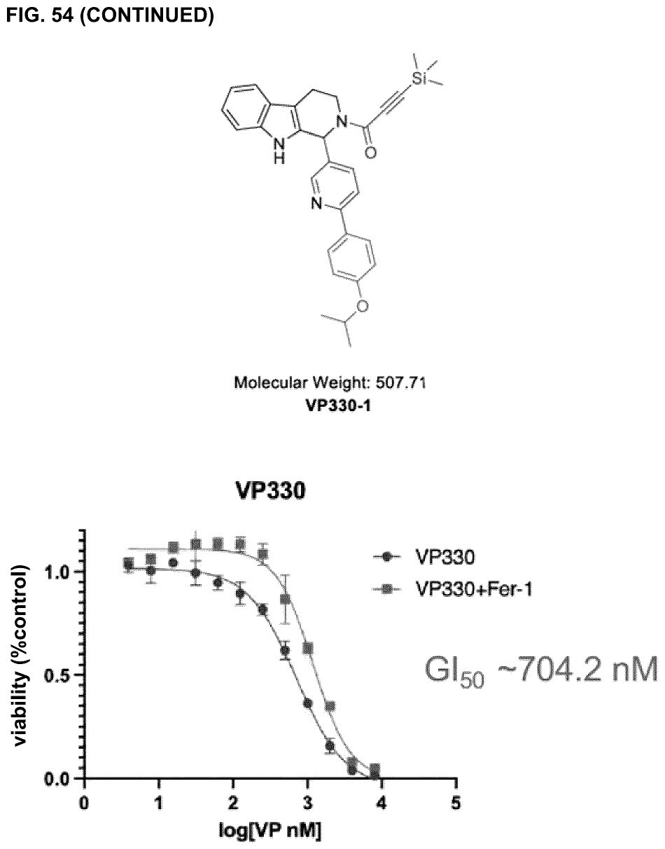

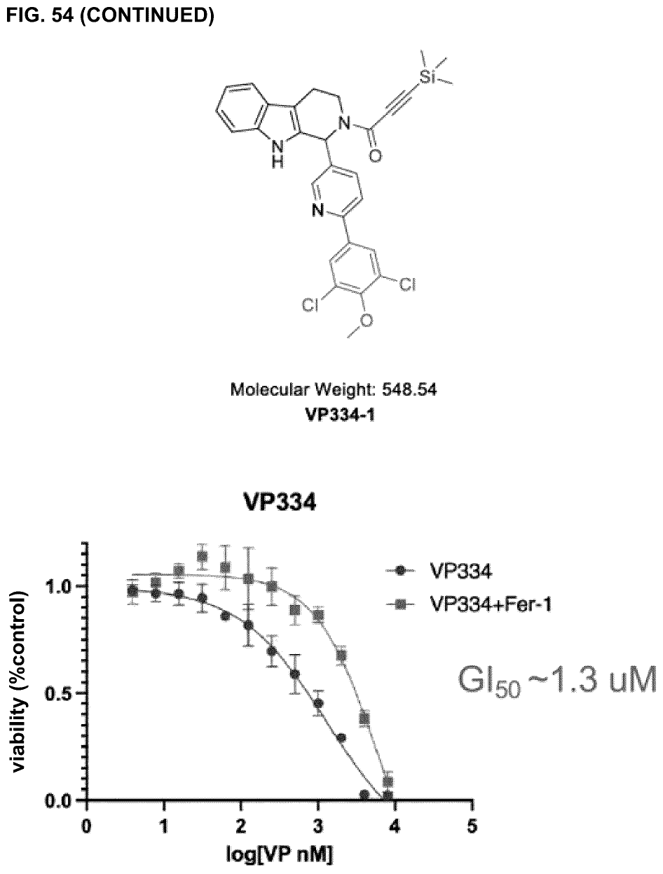

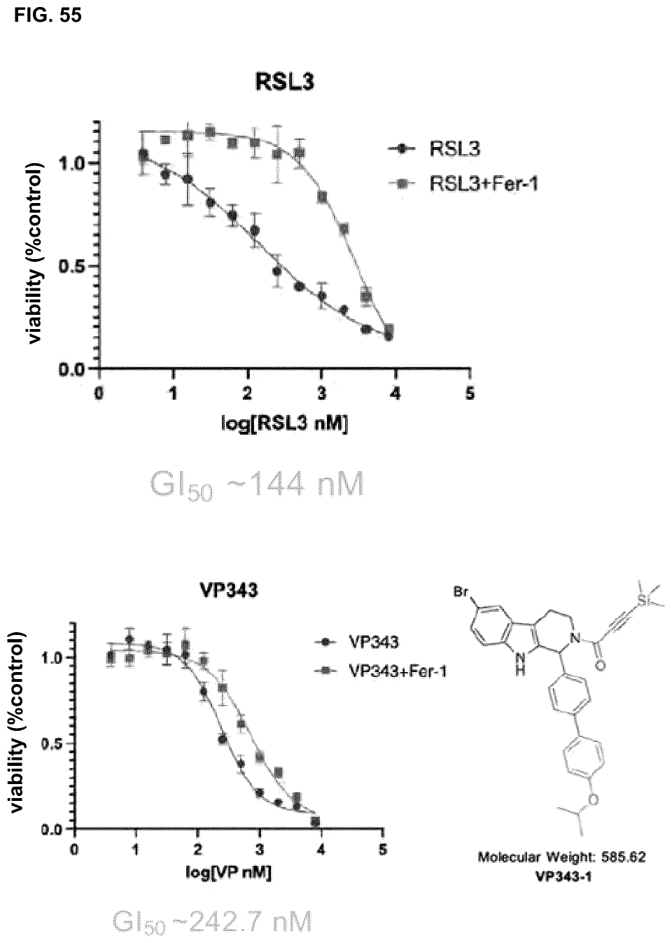

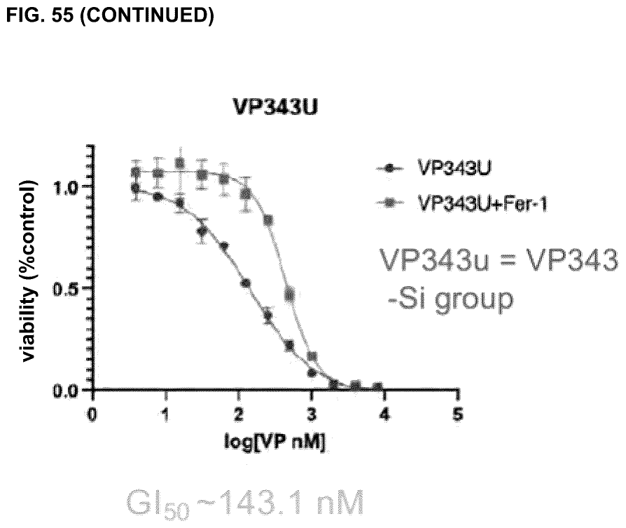

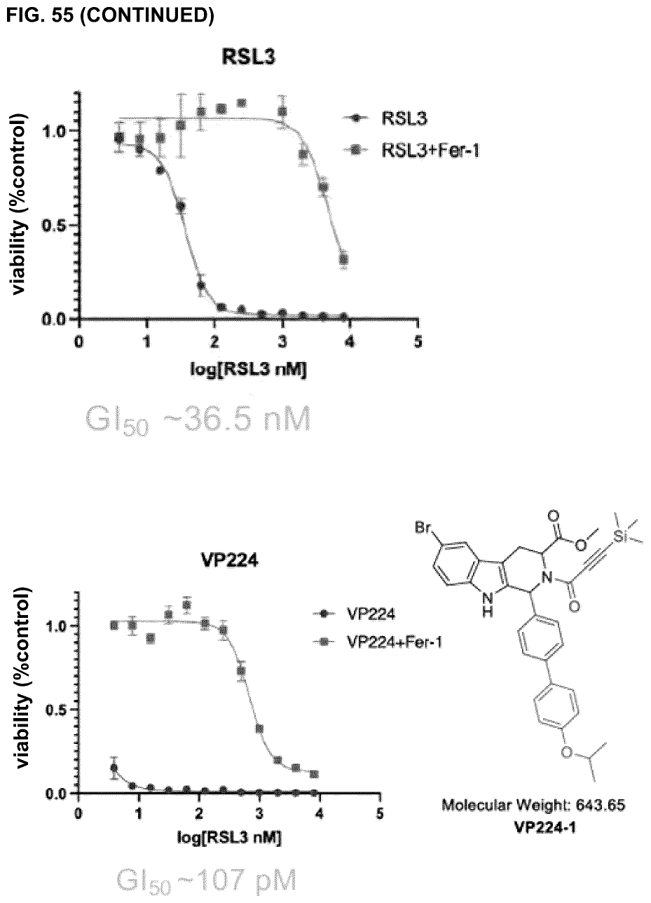

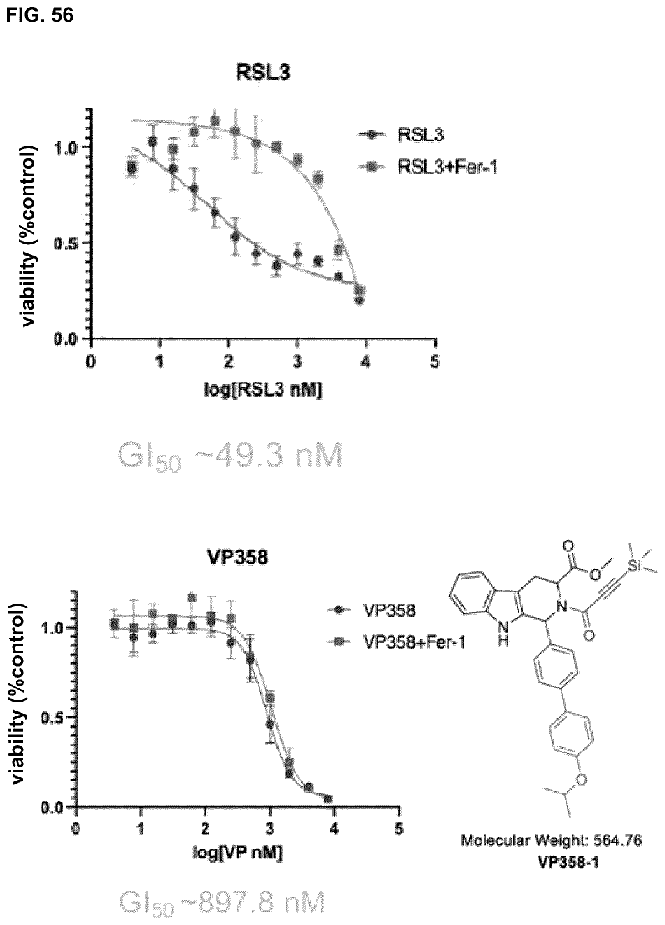

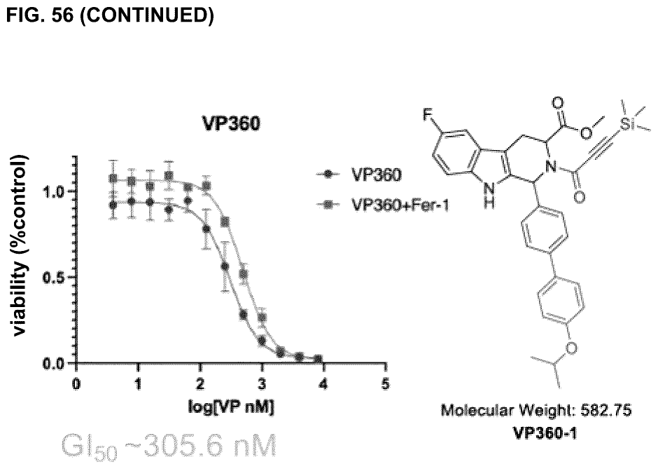

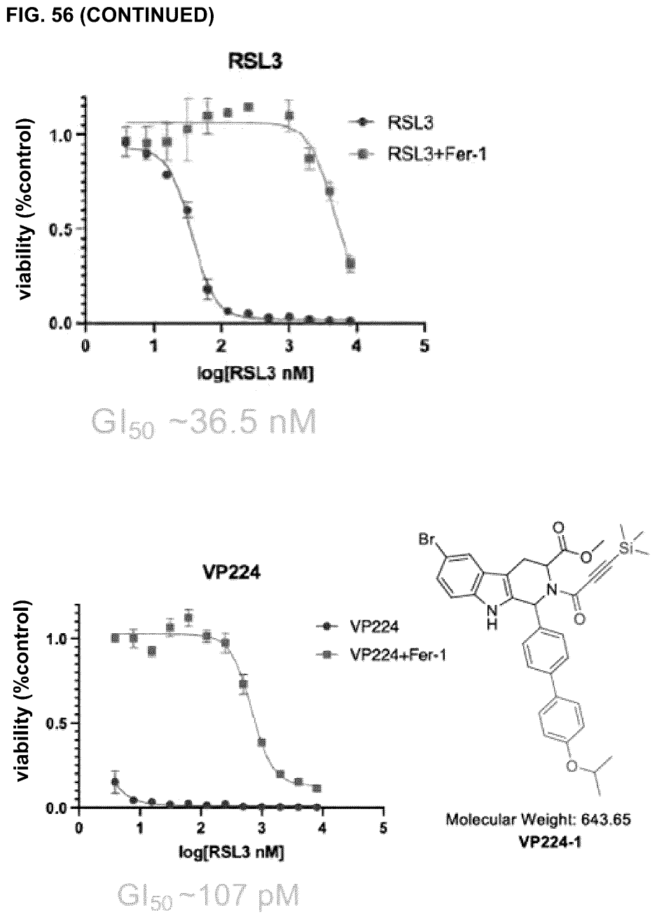

Based on the latest results, we performed a systematic investigation in order to evaluate the role of both ester and bromine in potency and selectivity (FIG. 52). VP-328 induced ferroptosis in HT-1080 fibrosarcoma cells with less potency than RSL3 (FIG. 53), while VP compounds without the —Br and methyl ester did not induce ferroptosis in HT-1080 cells (FIG. 54). VP-224 derivatives without methyl ester (e.g., VP-343) did not induce ferroptosis in HT-1080 cells, and the one with unmasked warhead (e.g., VP-343U) had greater potency in cells (FIG. 55). VP-224 derivatives without 5-Br (VP-358) or adding 5-F (VP-360) did not induce ferroptosis in HT-1080 cells, and fluorine improved the potency in cells, but not comparable to bromo (FIG. 56).

### Example 6

### Testing Compounds Selectivity in Drug-Resistant Persister Cells

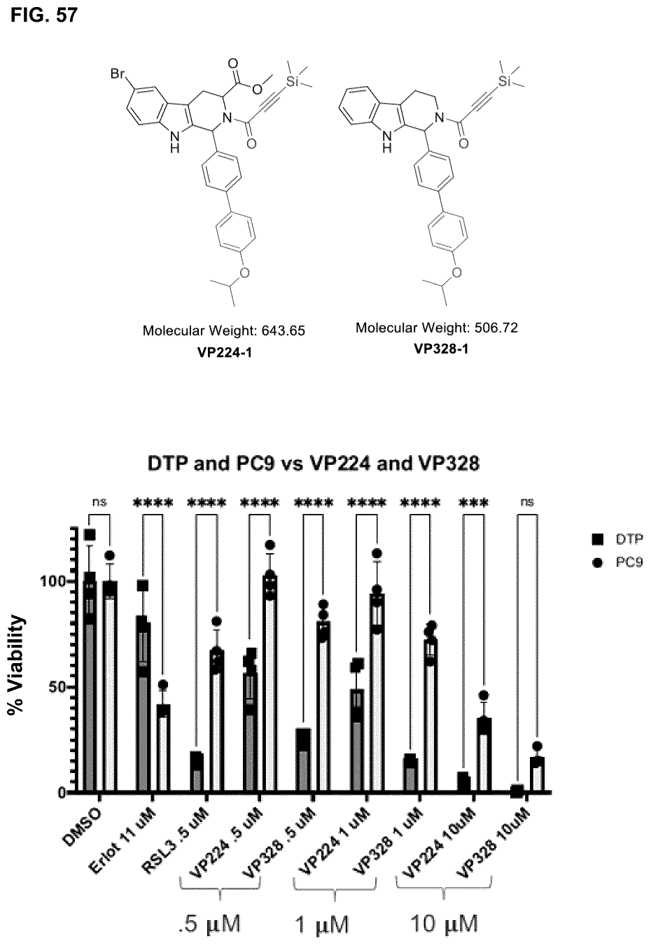

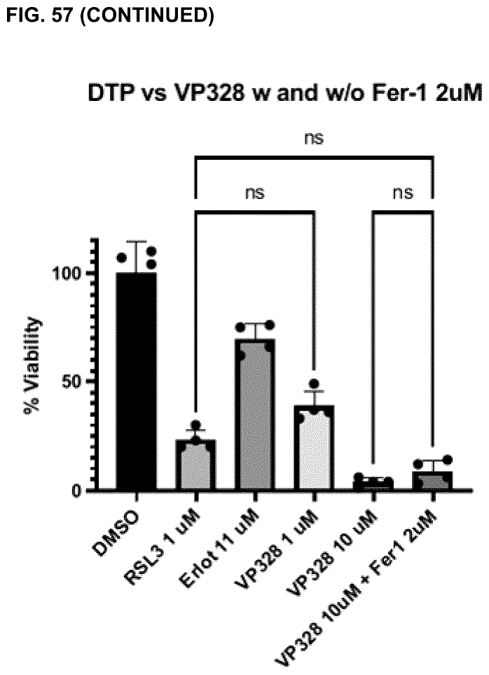

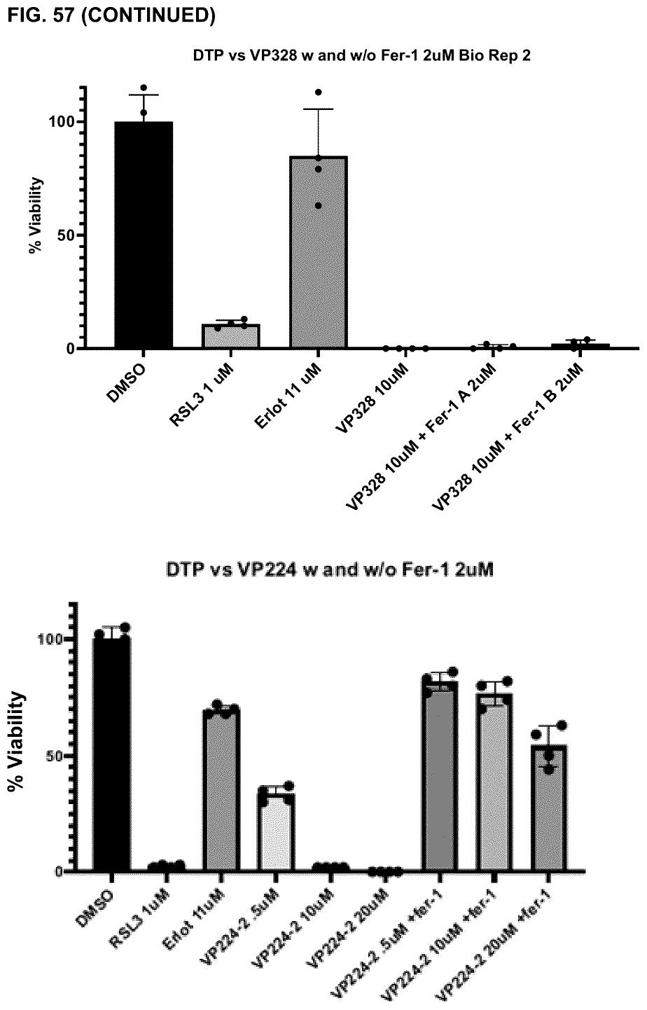

Drug Tolerant Persisters (DTPs) are a subpopulation of cells implicated in post chemotherapy relapse across multiple cancer types, and are known to be specifically sensitive to GPX4 inhibition relative to their parental cell line of origin (FIGS. 7A-7B). We demonstrated that the LOC1886 analog QW-314 specifically induced ferroptosis in DTPs but not parental PC9s (FIG. 7C), as does RSL3 analog VP-224 (FIG. 57) at dosage comparable to or slightly better than RSL3.

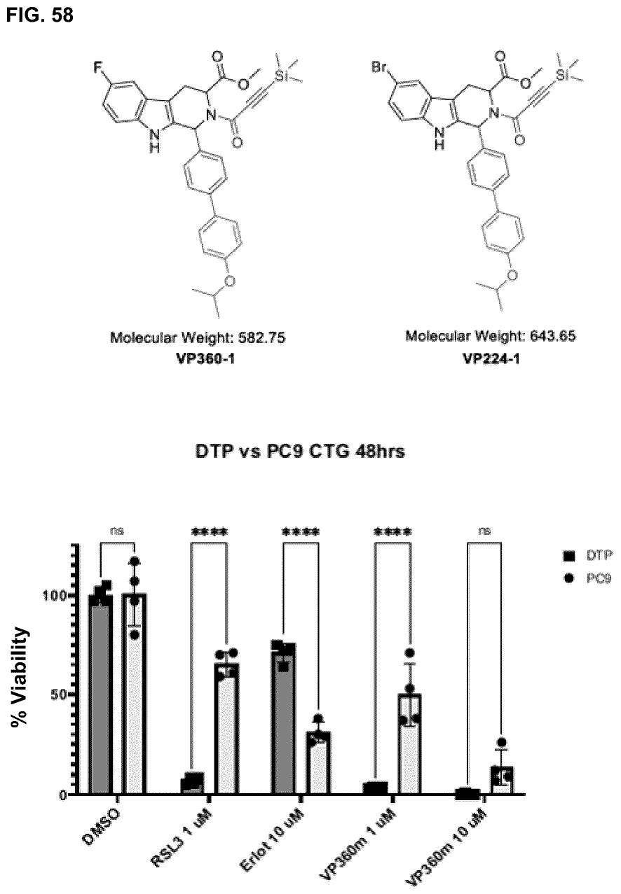

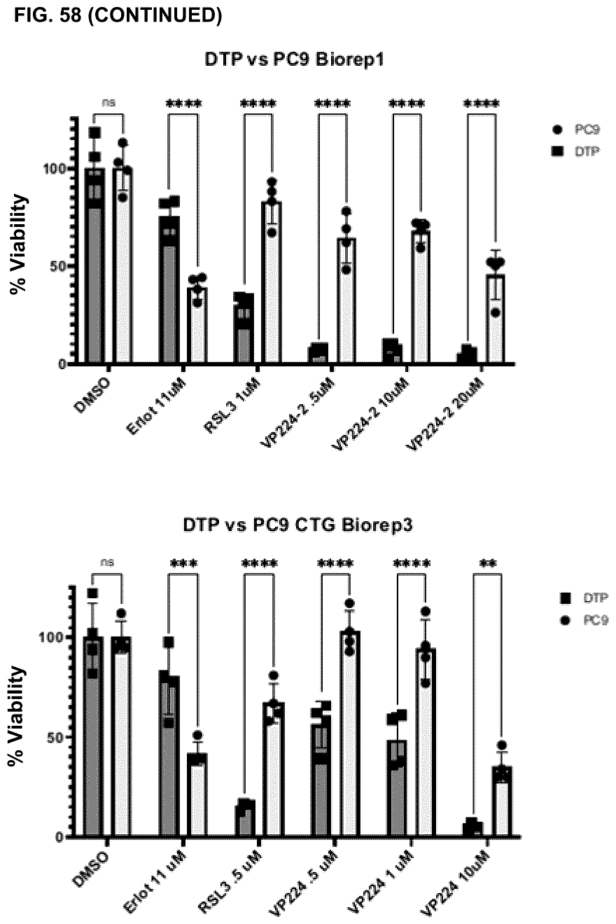

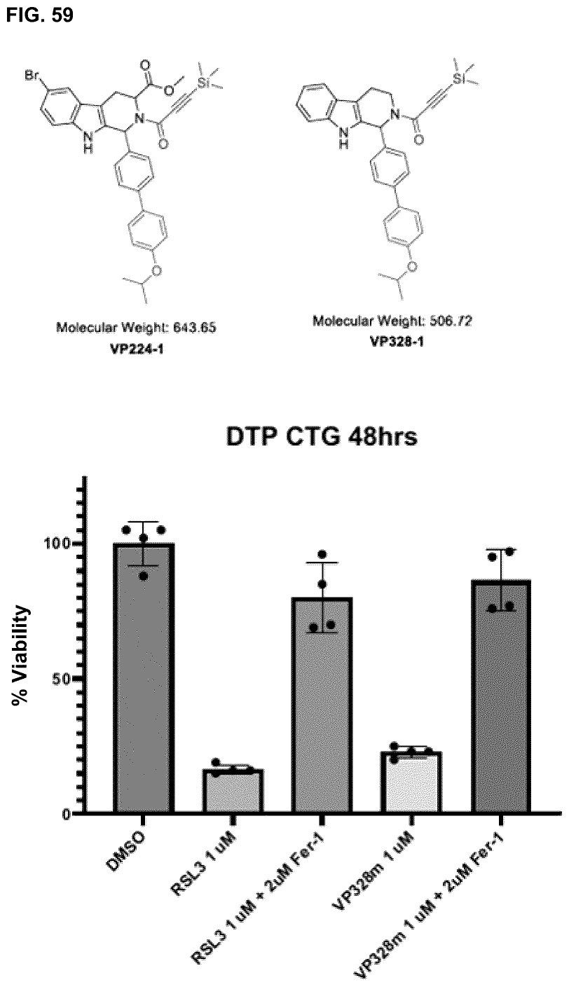

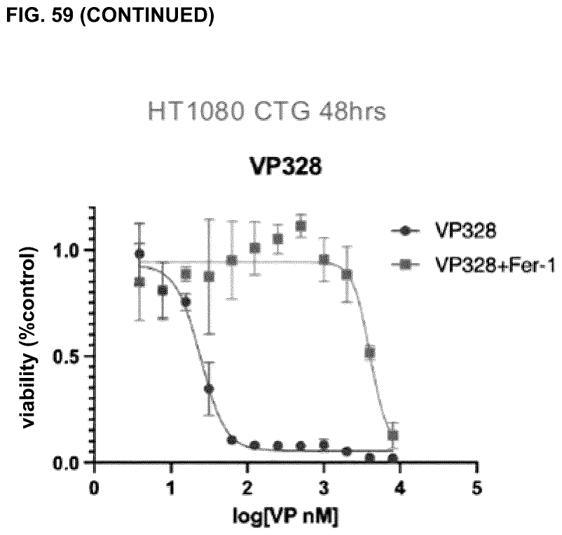

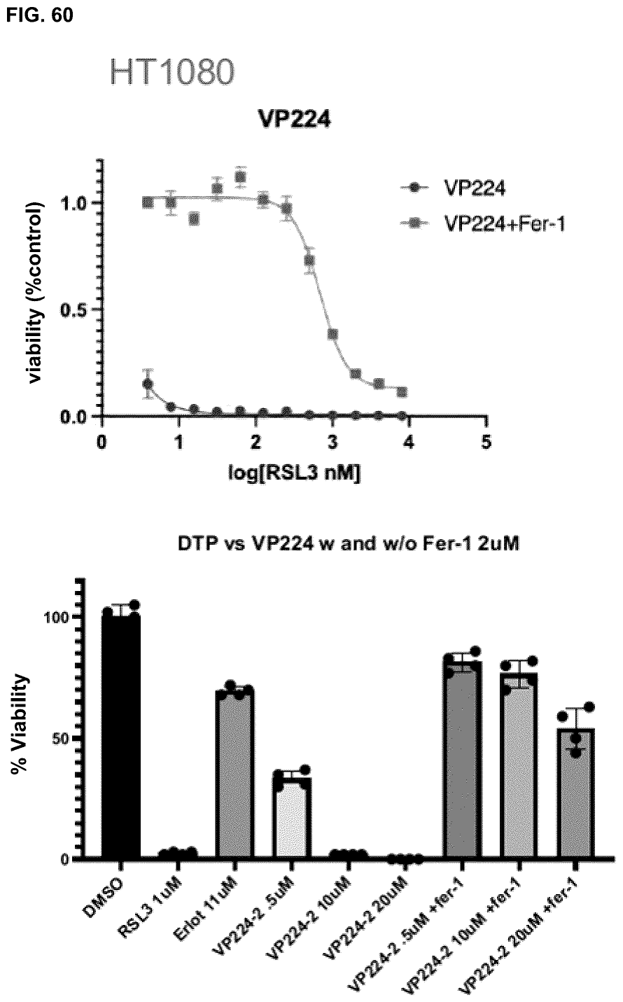

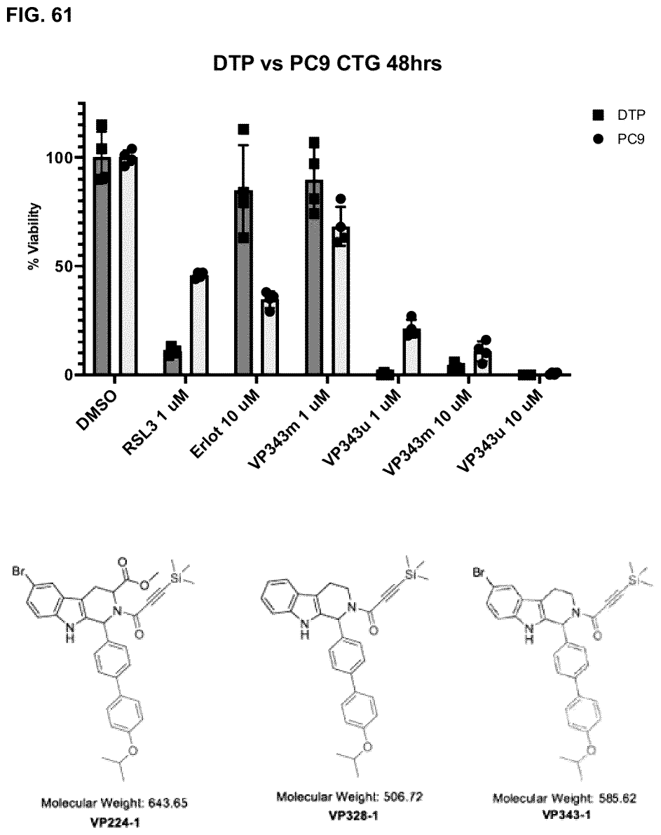

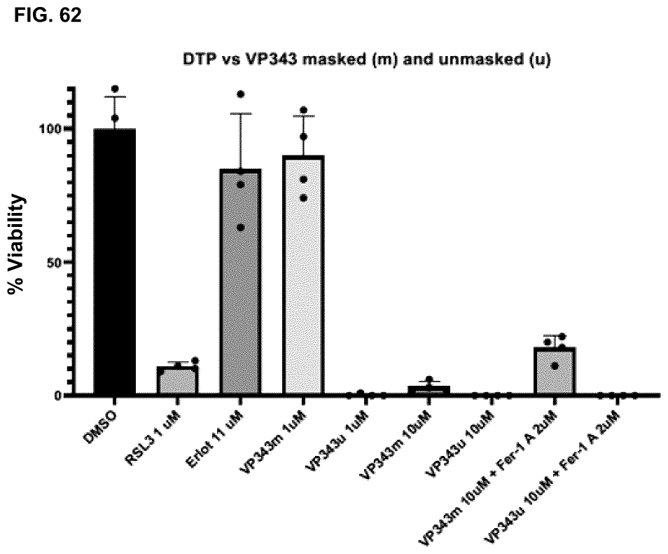

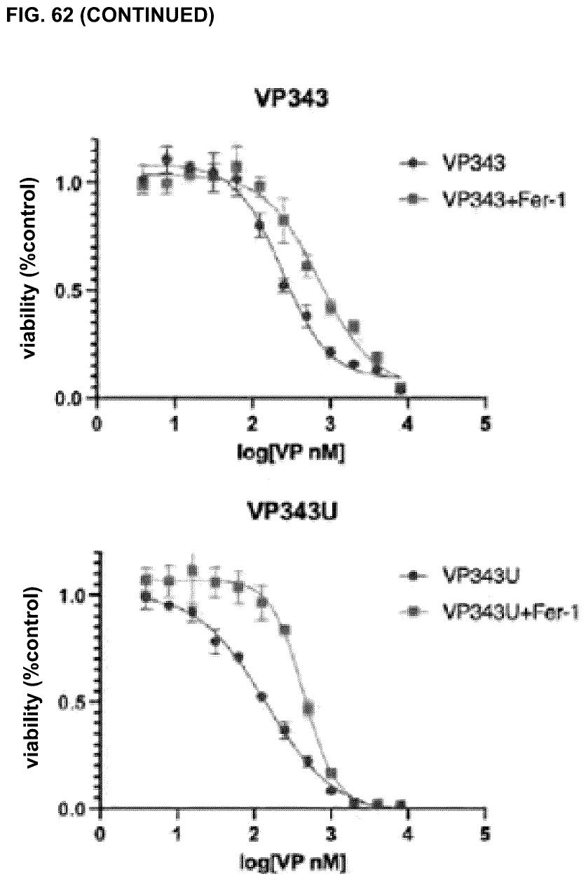

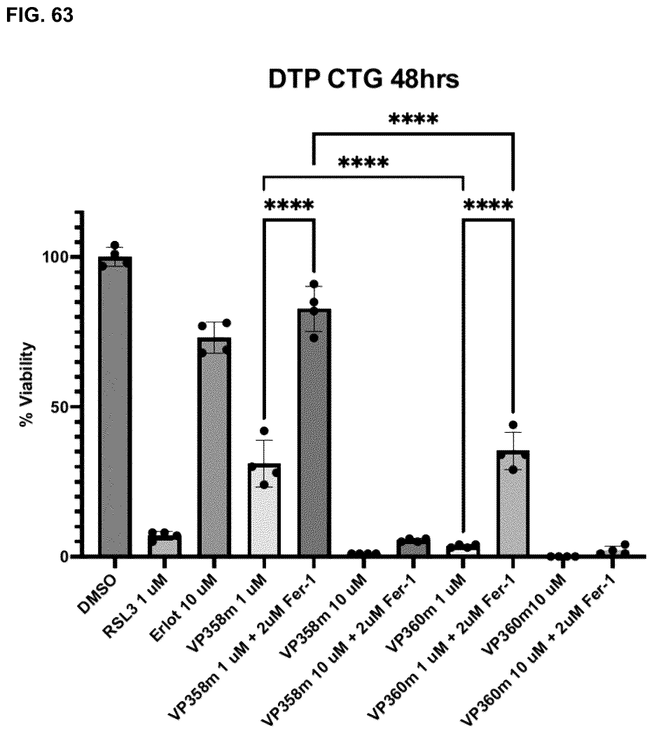

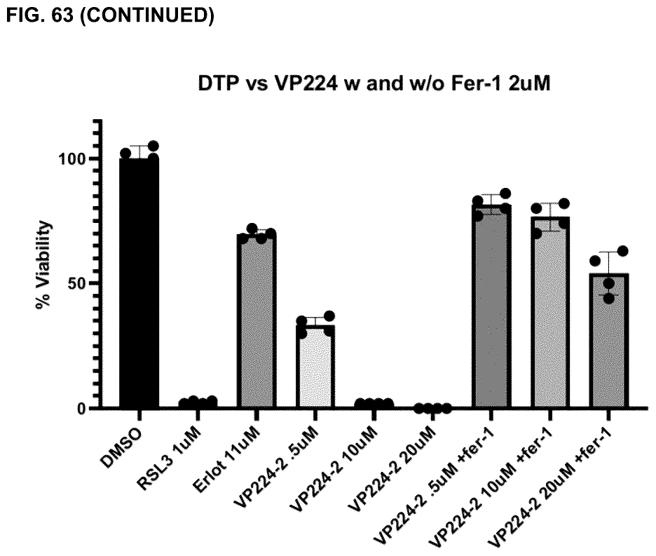

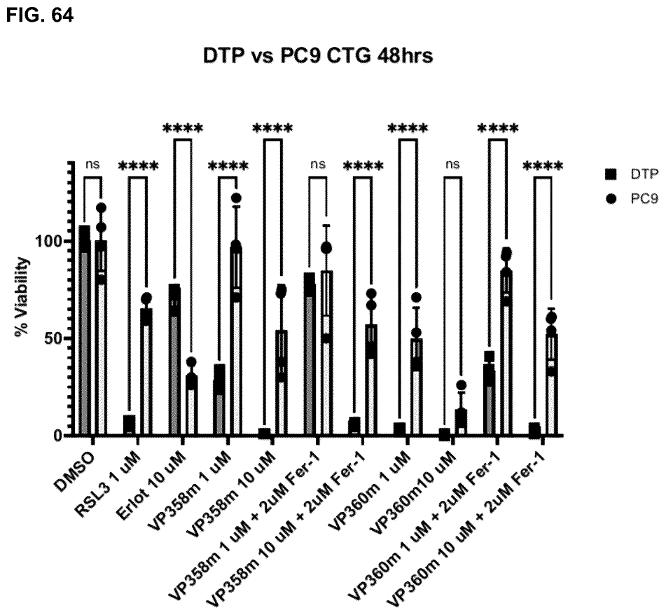

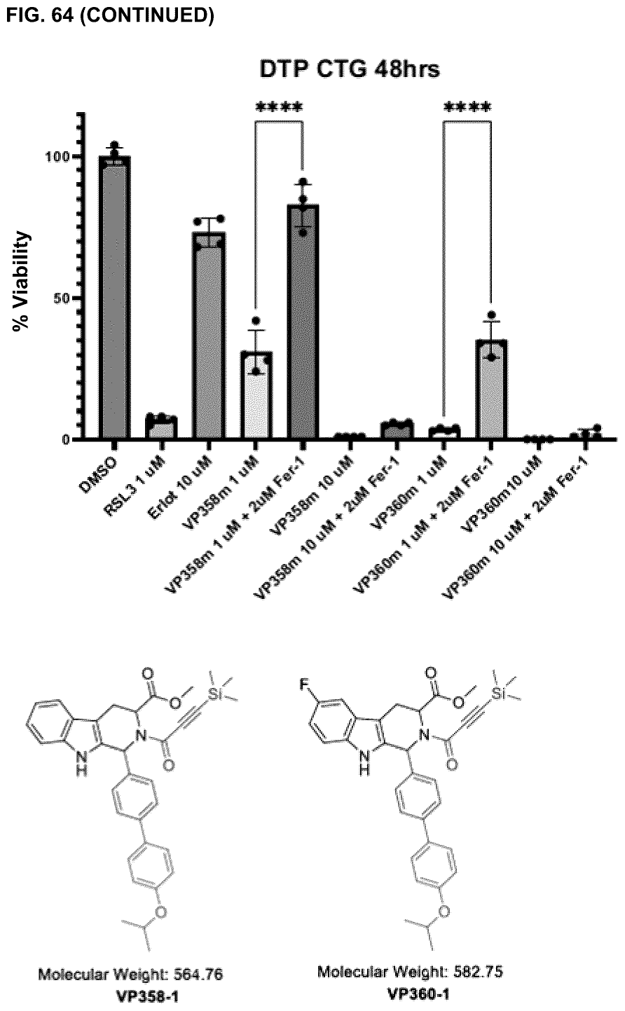

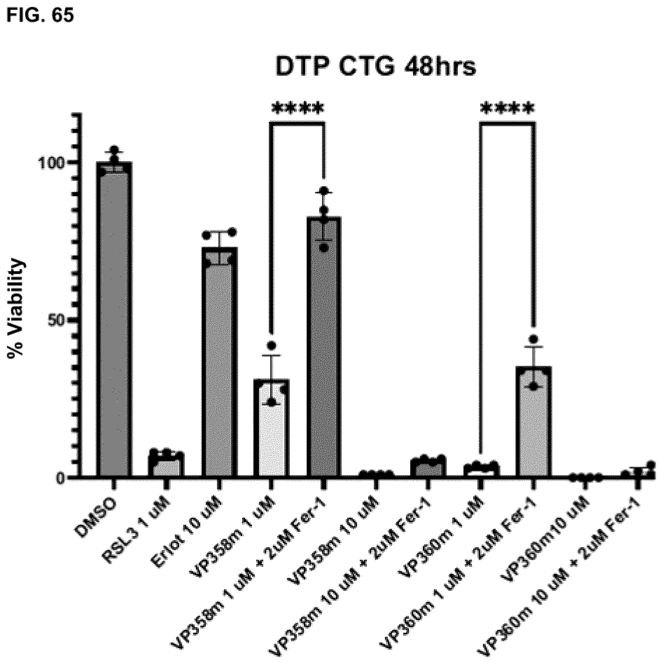

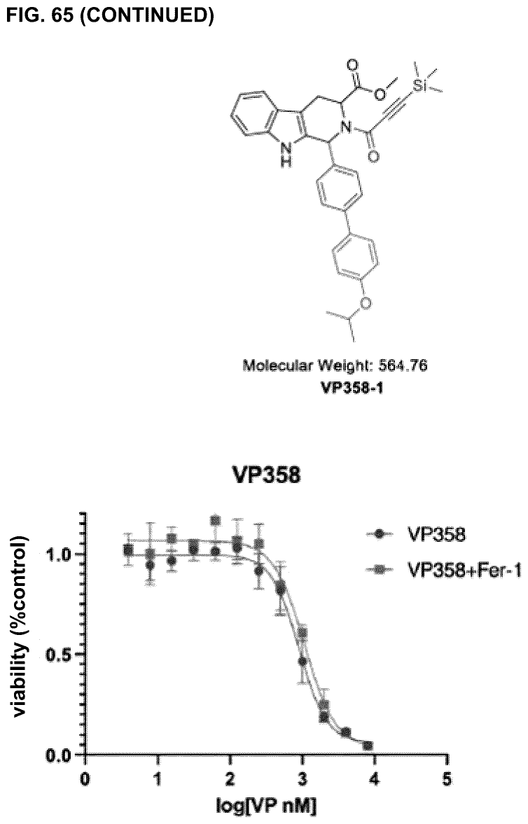

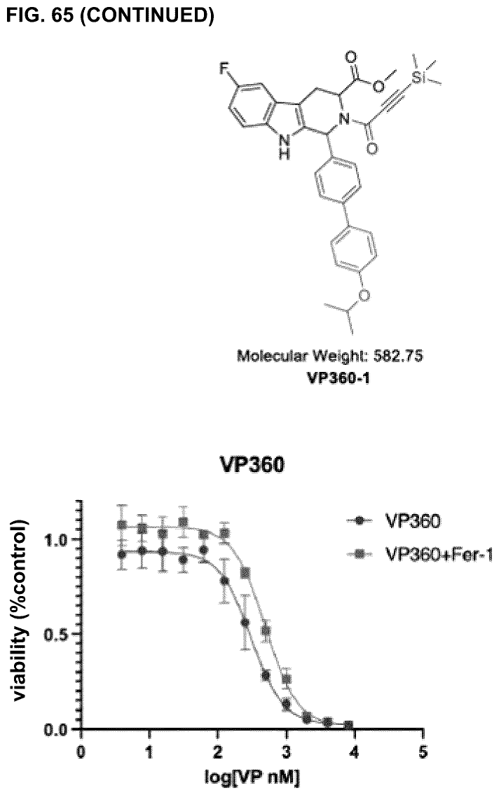

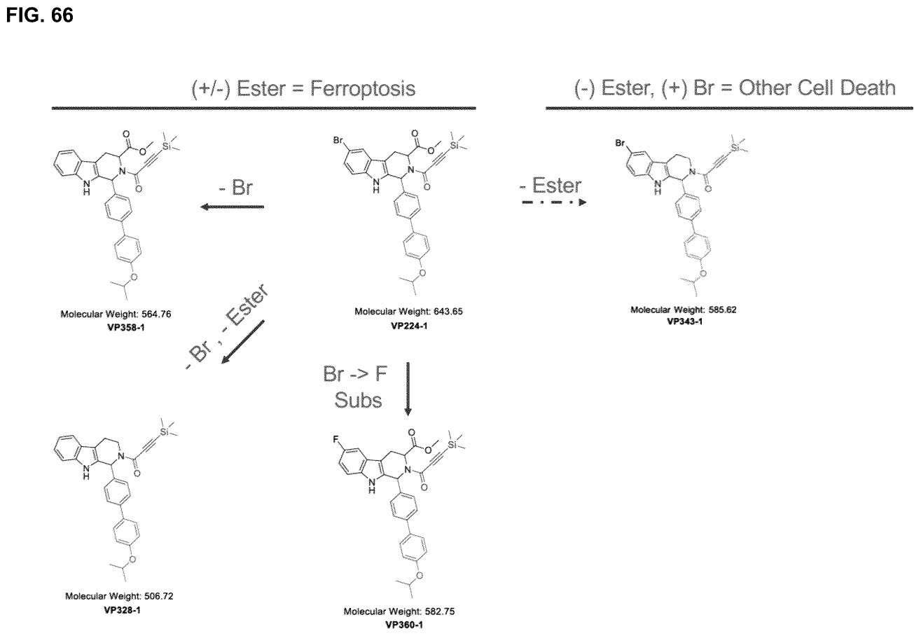

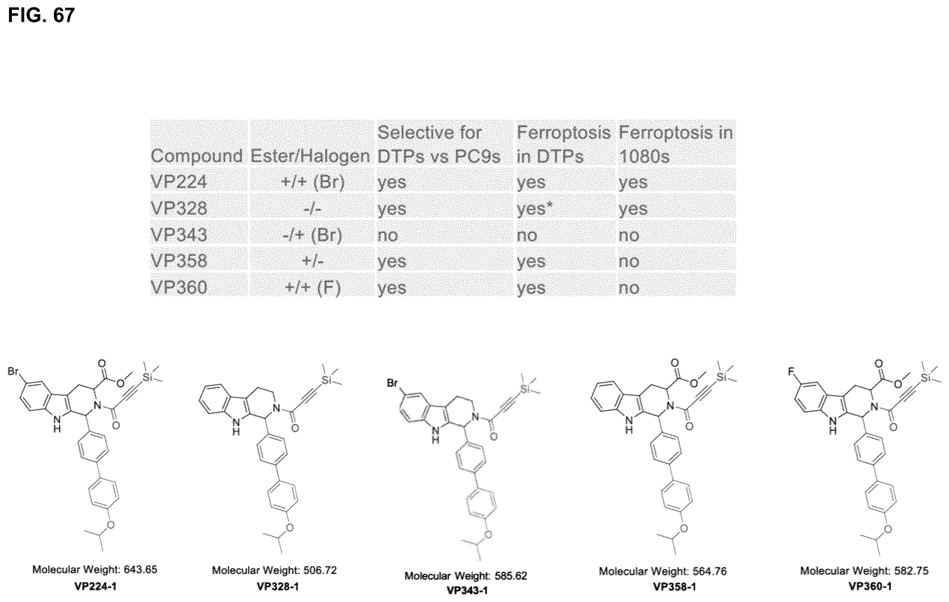

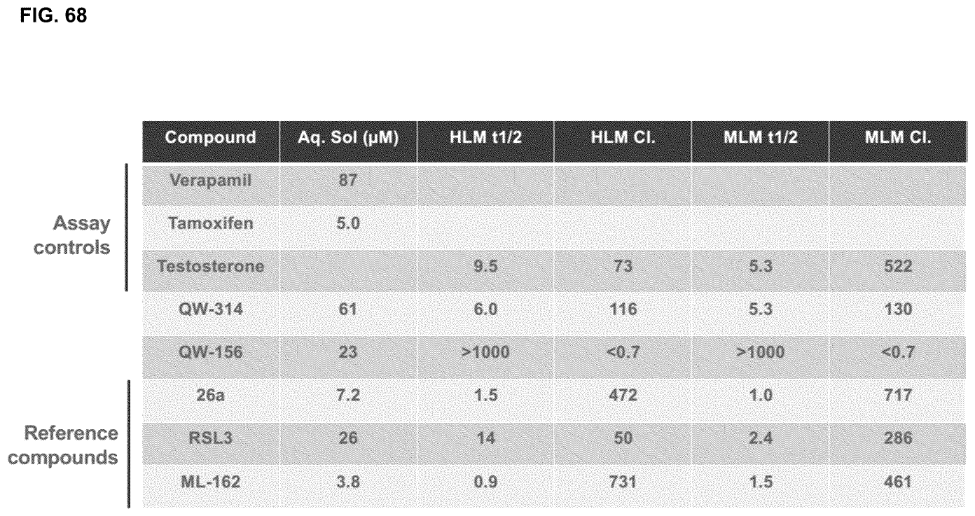

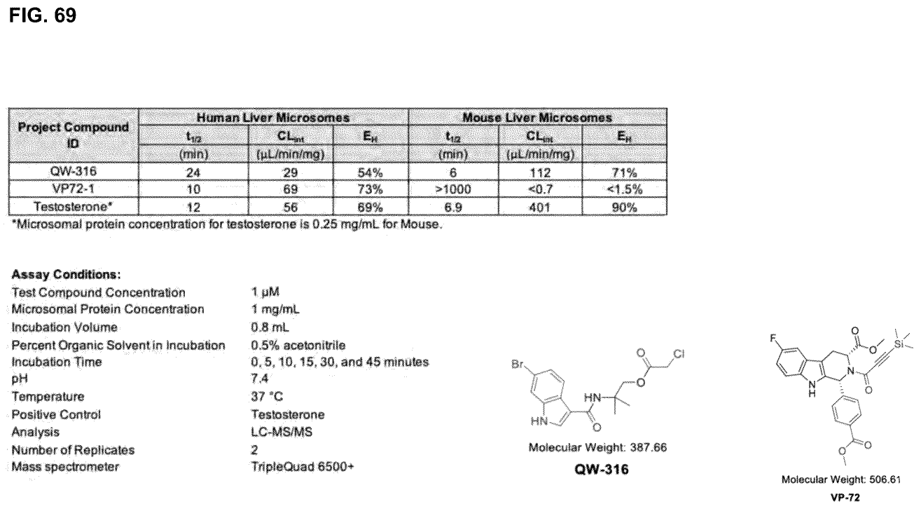

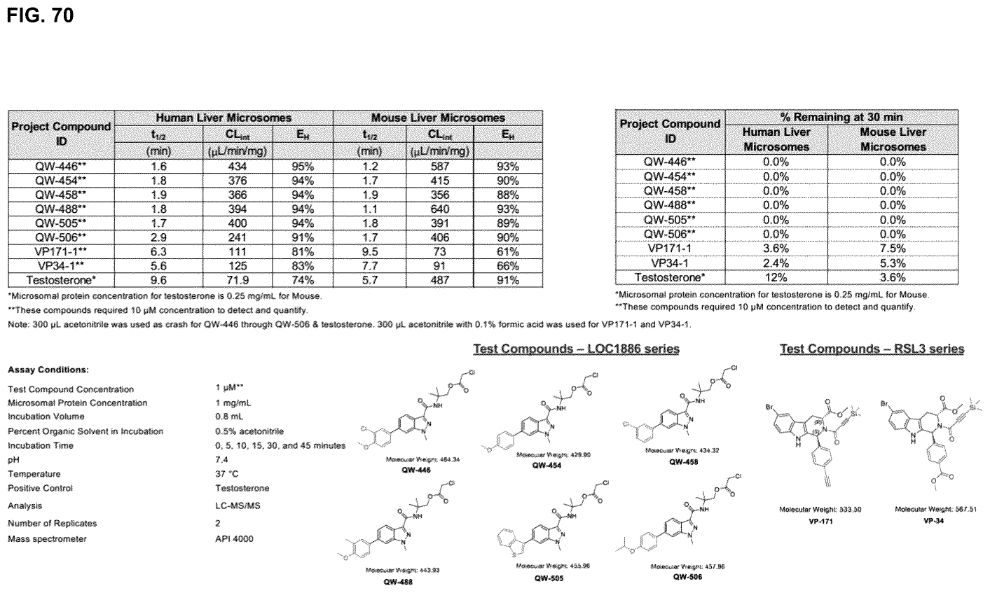

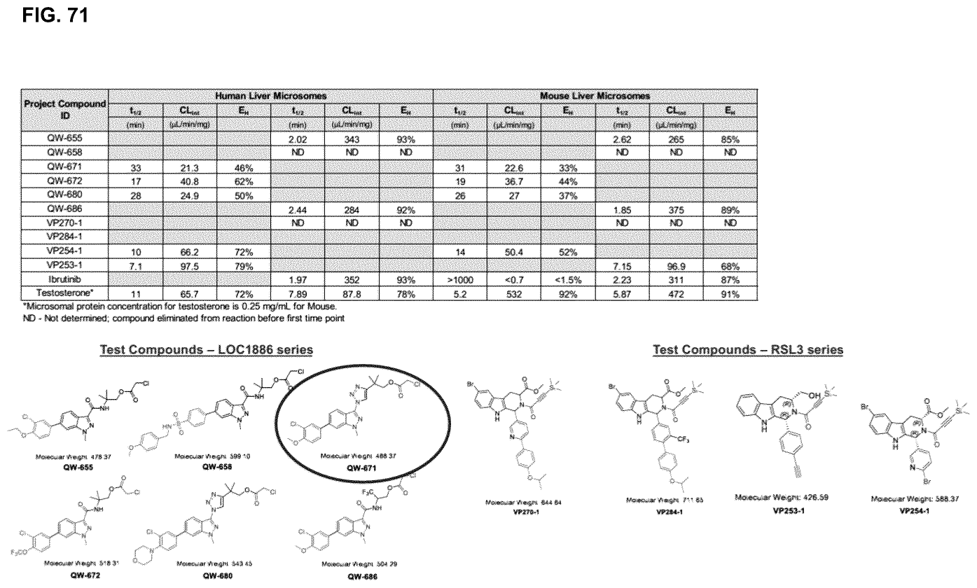

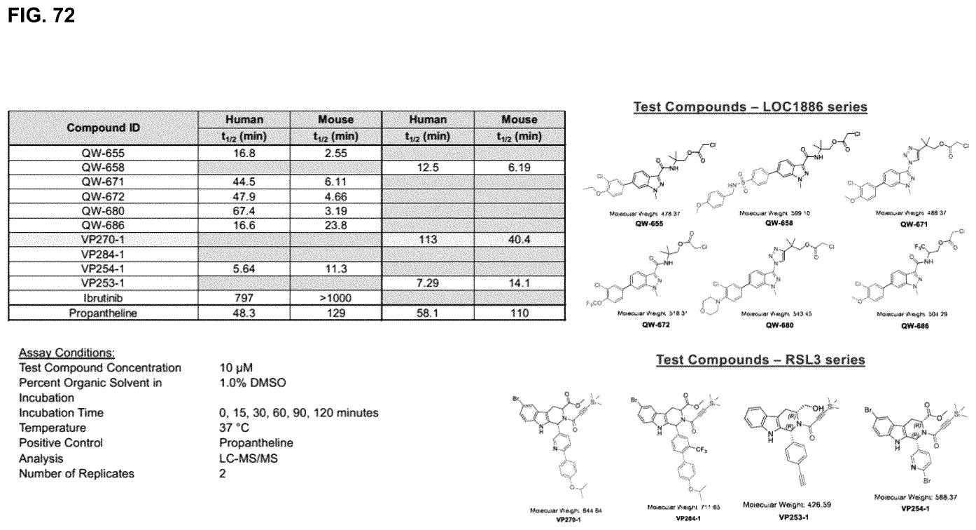

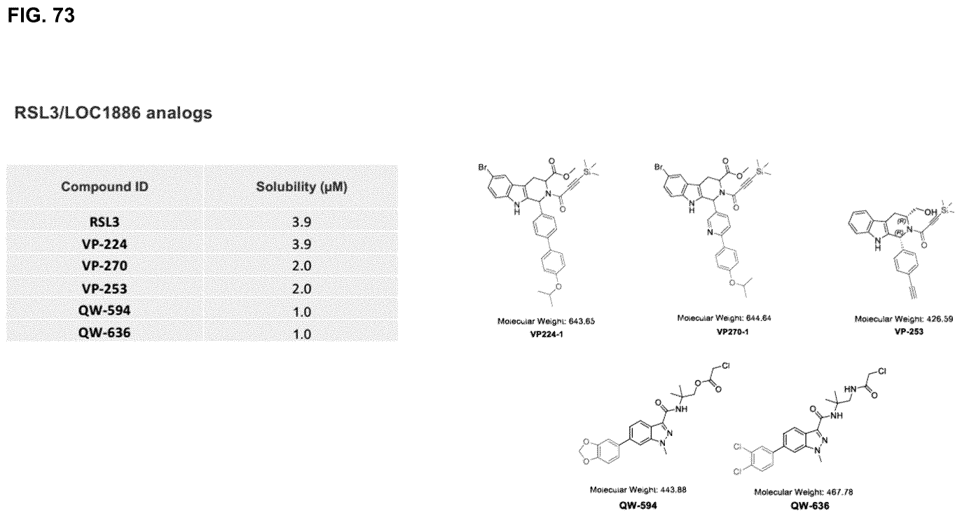

Moreover, we identified that in the RSL3 scaffold of compounds, in the absence of a halogen the ester is sufficient, but not necessary, for ferroptosis induction in DTPs, but in the presence of a halogen, the ester is necessary for ferroptosis in both DTPs and HT1080s. Notably, removal of the ester blunted the ability of ferrostatin-1 (fer-1) to rescue VP compound induced ferroptosis in DTPs. Additionally, although halogen substitution (e.g., Br→F) resulted in only minor potency changes in DTPs (FIG. 58), halogen presence and type had substantial effects on both potency and especially on the ability of fer-1 to rescue DTP cell death (FIGS. 59-63). Opposing effects of VP-224 analogs VP-358 and VP-360 in HT1080s vs. DTPs also yielded novel insight into the mechanism of fer-1 rescue from GPX4 inhibition induced ferroptosis (FIGS. 64-69). Additional test results for selected LOC1886 and RSL3 analogs are shown in FIG. 70-73.

### Example 7

### In Vivo Pharmacokinetic Properties of Lead Compounds

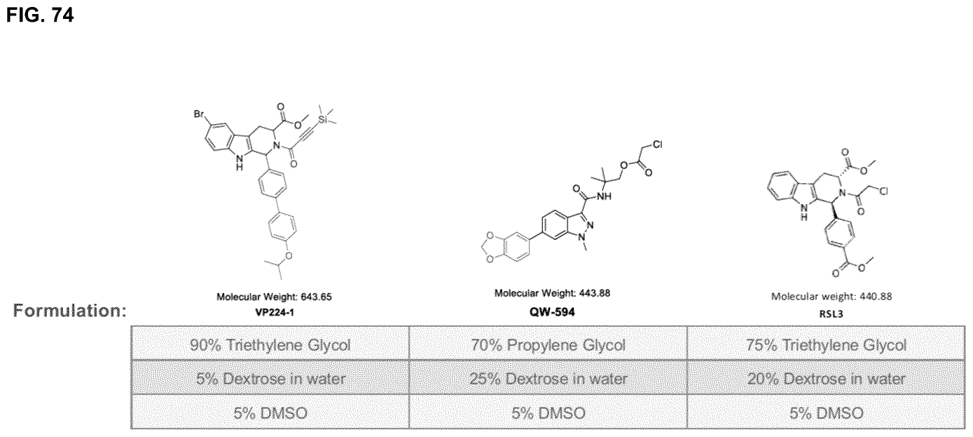

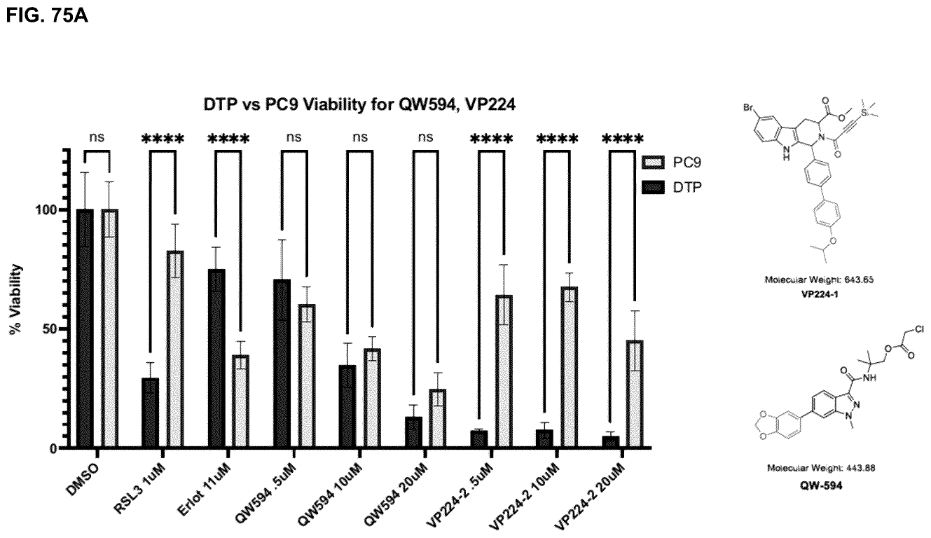

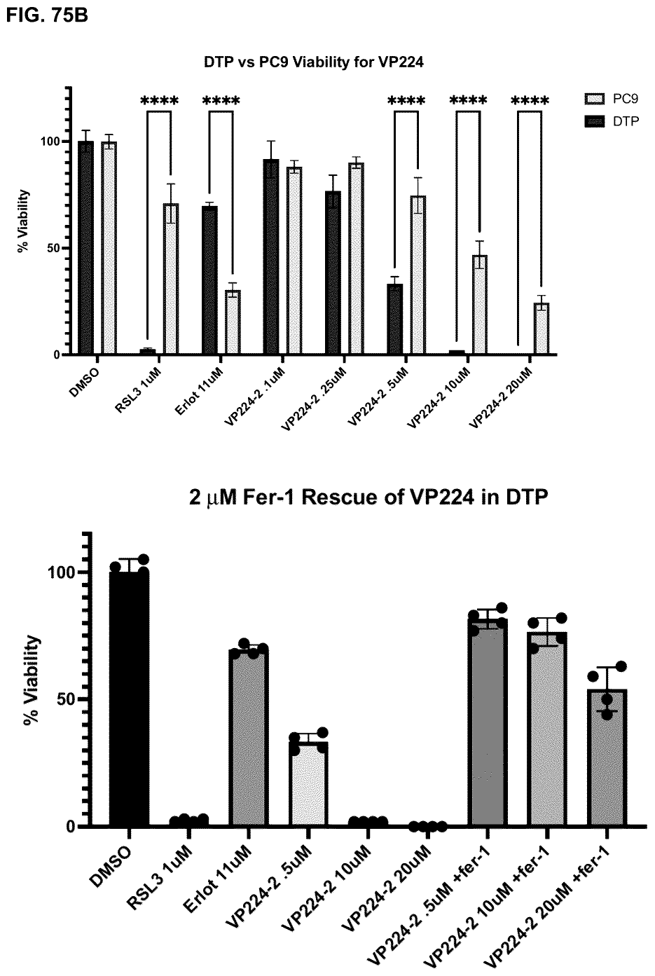

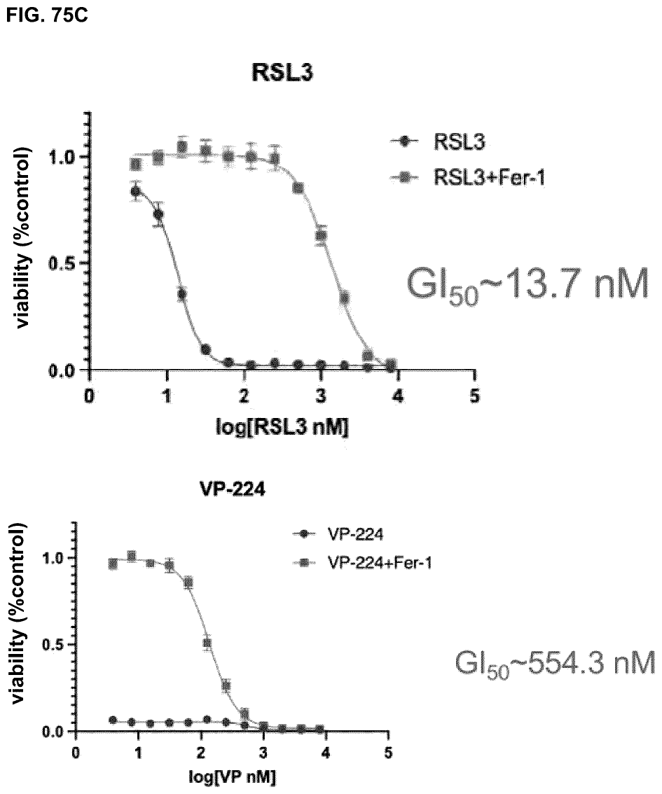

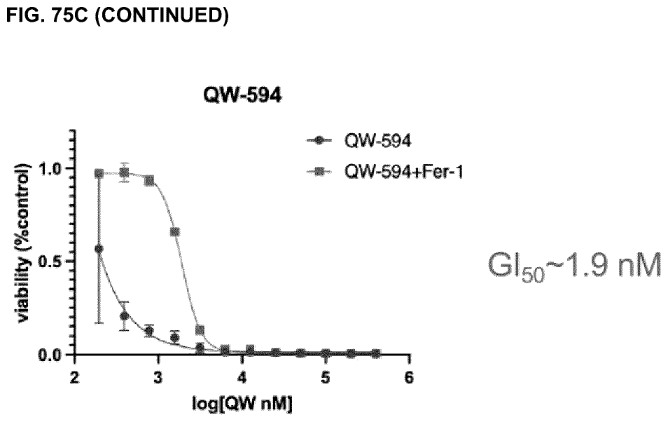

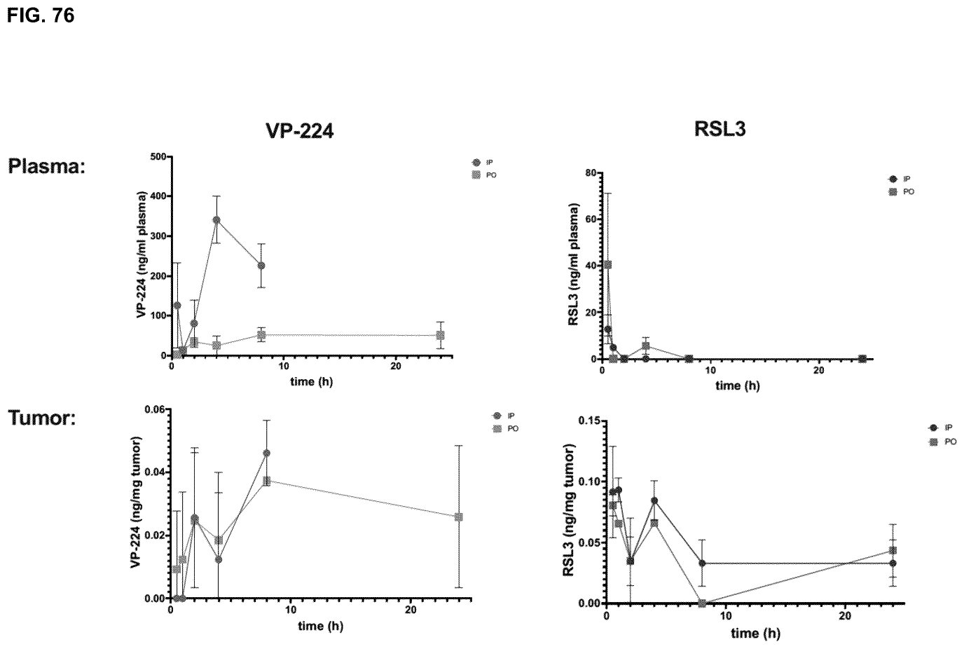

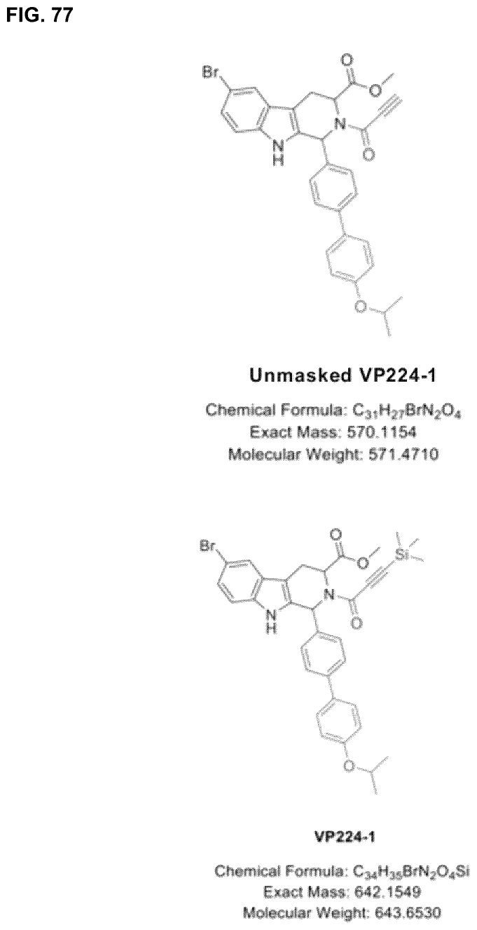

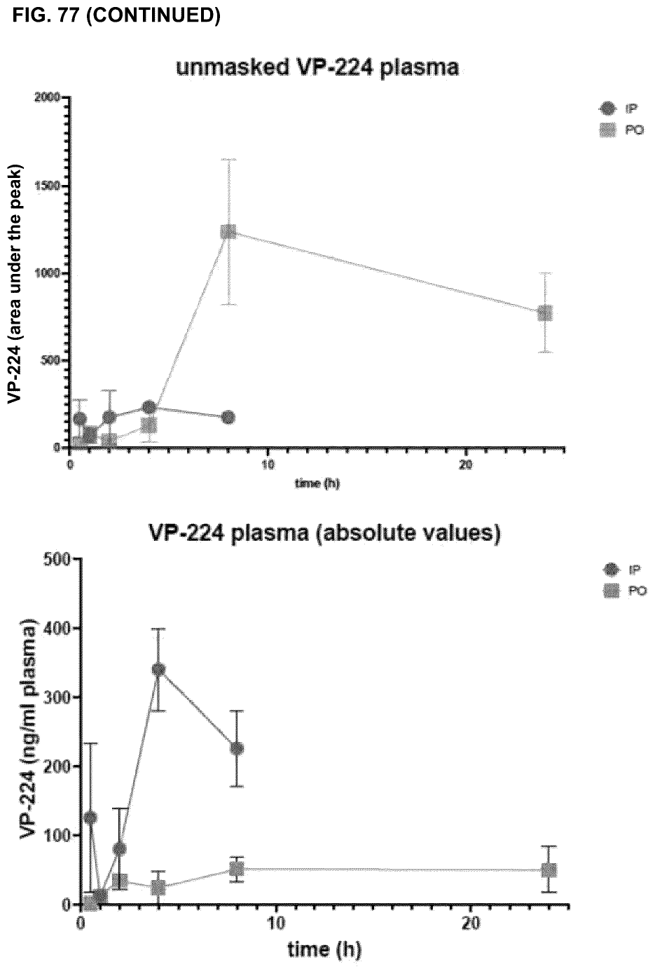

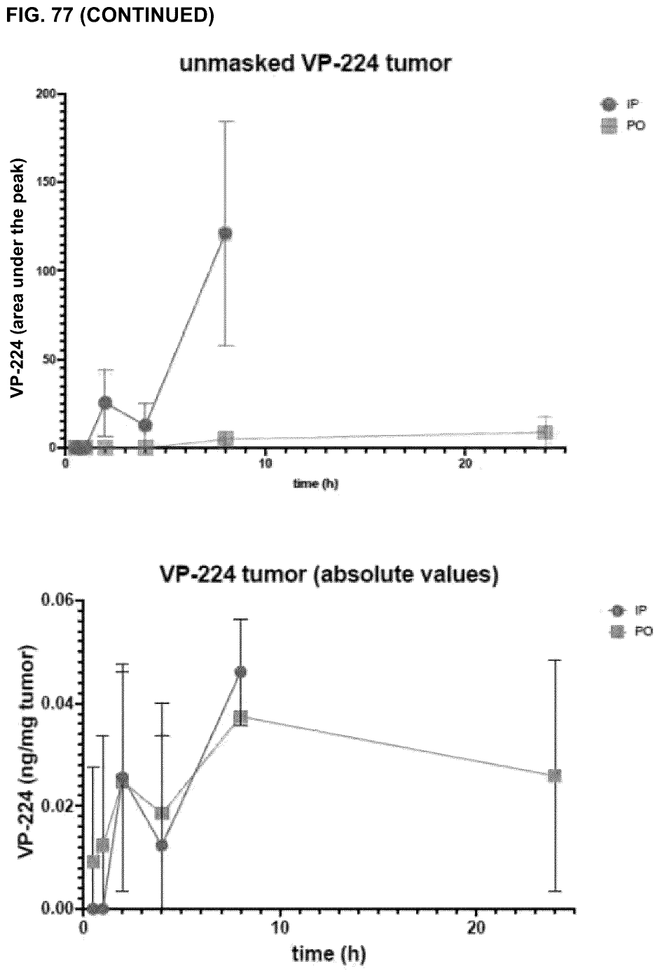

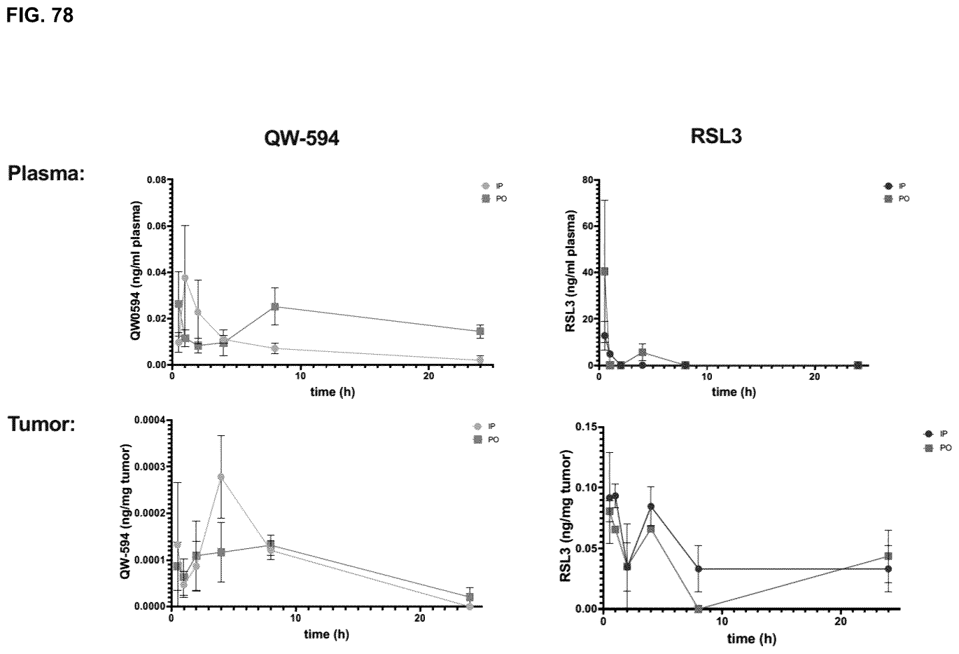

We tested two of our lead GPX4 inhibitors, one lead compound from each scaffold, alongside with RSL3 for their pharmacokinetic stability in large-cell lymphoma tumor-bearing mice. VP-224 and QW-594 were administered to mice intraperitoneally (IP) or orally (PO) and the compound concentration was measured in the mice plasma and tumors in several timepoints post administration (FIG. 74). These results were compared to the same administration modes of RSL3. VP-224 but not QW-594 was both selective and lethal in DTPs compared to PC9s (FIG. 75A), and VP-224 was both selective and lethal in DTPs vs PC9s at >500 nM and was substantially rescued by Fer-1 (FIG. 75B). Moreover, VP-224 and QW-594 both induced ferroptosis in SU-DHL-6 B cell lymphoma cells (FIG. 75C). These studies pointed to an improved plasma stability of VP-224 compared to the original compound, RSL3, and a comparable tumor accumulation (FIG. 76). Interestingly, the unmasked form of VP-224 is more prominent in the plasma when administered orally compared to IP. In contrast, this form is significantly higher in the tumor when administered via IP compared to PO (FIG. 77). QW-594 was found less stable in plasma and tumor compared to RSL3 (FIG. 78). Further optimization of the vehicle formulation may improve on compounds pharmacokinetics.

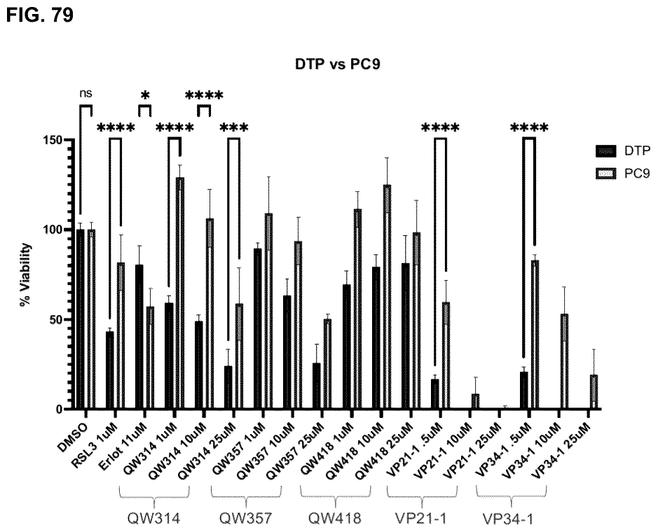

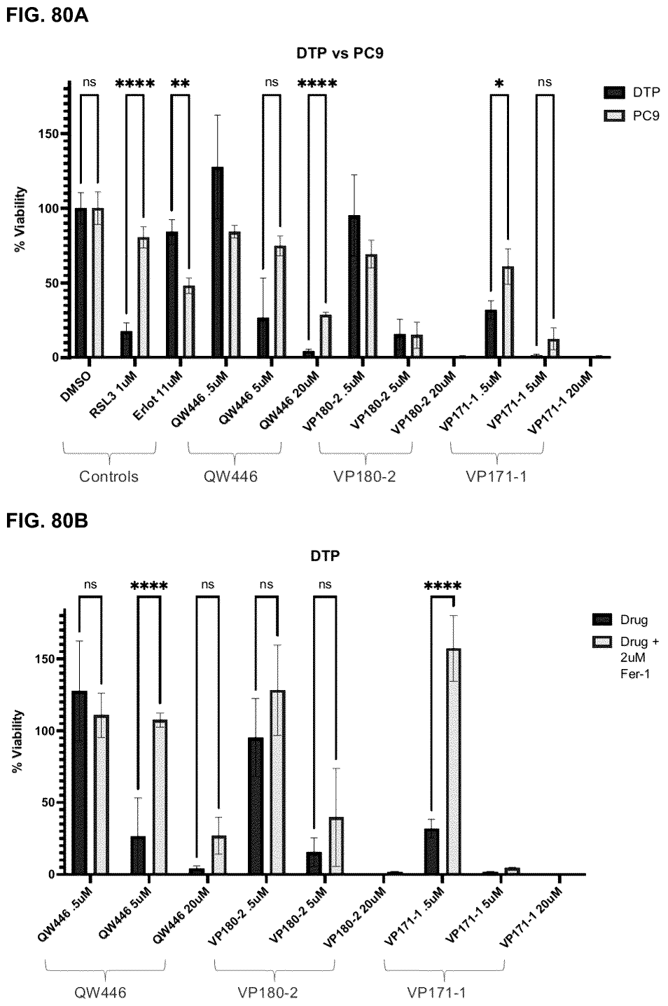

Overall, we made significant progress in the development of potent and selective GPX4 allosteric inhibitors in both chemical scaffolds (FIG. 79 and FIGS. 80A-80B). Our efforts hold the potential for the development of a novel therapeutics for treatment of drug-resistant cancers.

### Example 8

### Synthetic Schemes for LOC1886 Analogs

This Example provides synthetic schemes for LOC1886 analogs disclosed herein.

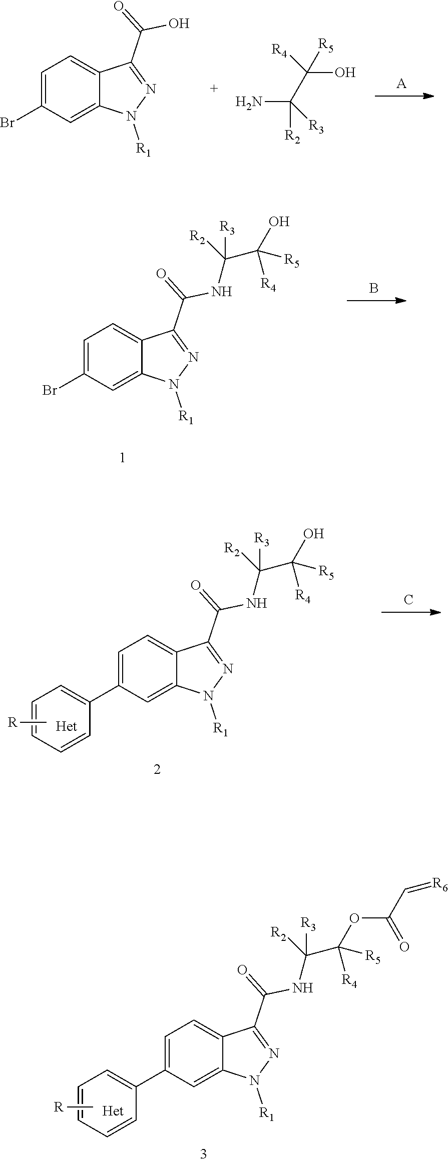

### General Synthetic Procedures

Procedure A: The appropriate amino alcohol (3 mmol) was dissolved in anhyd. THF (12 mL) and then EDC (3 mmol), HOBt (3 mmol) and the corresponding N-substituted 6-bromo-1H-indazole-3-carboxylic acid (2 mmol) were added sequentially at 25° C. After stirring overnight at r.t., evaporating the solvent, pouring the product into sat. aq NaHCO₃, extracting with EtOAc, washing with 1 N HCl, sat. aq NaHCO₃, brine, drying over Na₂SO₄ and concentrating the combined extract gave the amide 1.

Procedure B: Compound 1 (0.1 mmol) and corresponding boronic acids (0.15 mmol) were placed in a 25 mL round-bottom flask, and toluene/ethanol (v/v=5:1) (3 mL) and sat. NaHCO₃ (3 mL) were added. The mixture was flushed by N₂ for 10 min. Then, Pd(dppf)Cl₂ (0.01 mmol) was added and the reaction mixture was allowed to react at 90° C. in an oil bath for 12 h. Then, the reaction mixture was cooled to room temperature and treated with H₂O and extracted with EtOAc. The combined organic layers were washed with brine and dried over anhydrous Na₂SO₄. After the removal of EtOAc, the residue was purified by column chromatography (silica gel, 0-100% EtOAc/hexanes gradient) to afford the product 2.

Procedure C: Acyl chloride (0.12 mmol, 1.2 eq.) was added dropwise to a stirred solution of compound 2 (0.1 mmol, 1 eq.) and triethylamine (26.4 μL, 0.15 mmol, 1.5 eq.) in dry DCM (1 mL). The reaction was stirred at 0° C. for 1 h, concentrated, and purified by flash column chromatography (silica gel, 0-100% EtOAc/hexanes gradient) to afford the title compound 3.

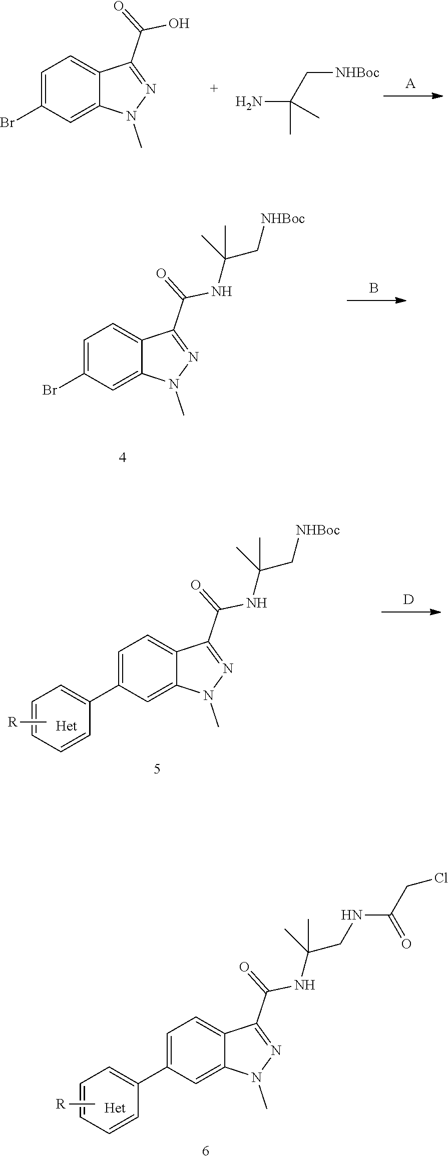

### General Synthetic Procedures

Procedure A: Tert-butyl (2-amino-2-methylpropyl) carbamate (3 mmol) was dissolved in anhyd. THF (12 mL) and then EDC (3 mmol), HOBt (3 mmol) and the corresponding 6-bromo-1-methyl-1H-indazole-3-carboxylic acid (2 mmol) were added sequentially at 25° C. After stirring overnight at r.t., evaporating the solvent, pouring the product into sat. aq NaHCO₃, extracting with EtOAc, washing with 1 N HCl, sat. aq NaHCO₃, brine, drying over Na₂SO₄ and concentrating the combined extract gave the amide 4.

Procedure B: Compound 4 (0.1 mmol) and corresponding boronic acids (0.15 mmol) were placed in a 25 mL round-bottom flask, and toluene/ethanol (v/v=5:1) (3 mL) and sat. NaHCO₃ (3 mL) were added. The mixture was flushed by N₂ for 10 min. Then, Pd(dppf)Cl₂ (0.01 mmol) was added and the reaction mixture was allowed to react at 90° C. in an oil bath for 12 h. Then, the reaction mixture was cooled to room temperature and treated with H₂O and extracted with EtOAc. The combined organic layers were washed with brine and dried over anhydrous Na₂SO₄. After the removal of EtOAc, the residue was purified by column chromatography (silica gel, 0-100% EtOAc/hexanes gradient) to afford the product 5.

Procedure D: Trifluoroacetic acid (1 mmol, 10 eq.) was added to compound 5 (0.1 mmol) in dichloromethane (5 ml). The mixture was stirred at r.t. for 12 h and concentrated in vacuo for further use. Chloroacetyl chloride (9.5 μL, 0.12 mmol, 1.2 eq.) was added dropwise to a stirred solution of above mixture and triethylamine (26.4 μL, 0.15 mmol, 1.5 eq.) in dry DCM (1 mL). The reaction was stirred at 0° C. for 1 h, concentrated, and purified by flash column chromatography (silica gel, 0-100% EtOAc/hexanes gradient) to afford the title compound 6.

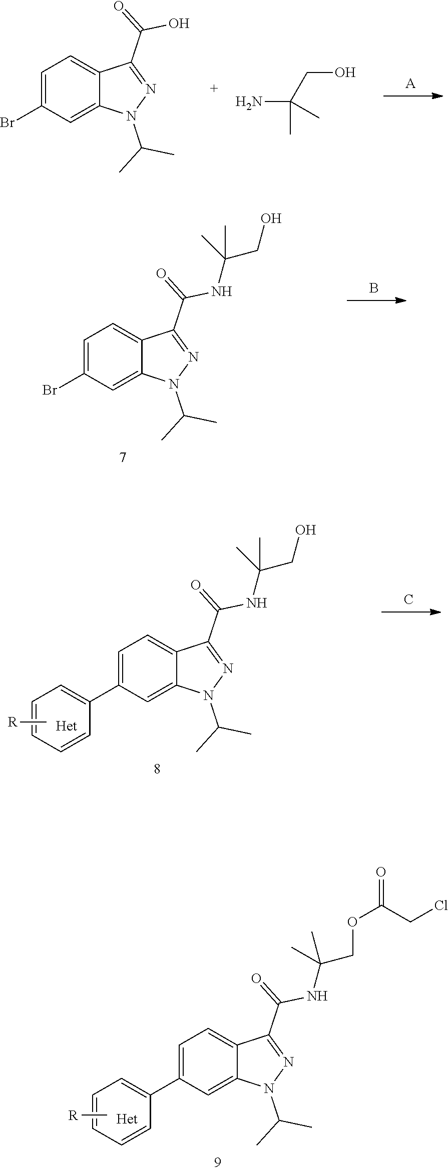

### General Synthetic Procedures

Procedure A: 2-Amino-2-methyl-1-propanol (3 mmol) was dissolved in anhyd. THF (12 mL) and then EDC (3 mmol), HOBt (3 mmol) and the corresponding 6-bromo-1-isopropyl-1H-indazole-4-carboxylic acid (2 mmol) were added sequentially at 25° C. After stirring overnight at r.t., evaporating the solvent, pouring the product into sat. aq NaHCO₃, extracting with EtOAc, washing with 1 N HCl, sat. aq NaHCO₃, brine, drying over Na₂SO₄ and concentrating the combined extract gave the amide 7.

Procedure B: Compound 7 (0.1 mmol) and corresponding boronic acids (0.15 mmol) were placed in a 25 mL round-bottom flask, and toluene/ethanol (v/v=5:1) (3 mL) and sat. NaHCO₃ (3 mL) were added. The mixture was flushed by N₂ for 10 min. Then, Pd(dppf)Cl₂ (0.01 mmol) was added and the reaction mixture was allowed to react at 90° C. in an oil bath for 12 h. Then, the reaction mixture was cooled to room temperature and treated with H₂O and extracted with EtOAc. The combined organic layers were washed with brine and dried over anhydrous Na₂SO₄. After the removal of EtOAc, the residue was purified by column chromatography (silica gel, 0-100% EtOAc/hexanes gradient) to afford the product 8.

Procedure C: Chloroacetyl chloride (0.12 mmol, 1.2 eq.) was added dropwise to a stirred solution of compound 8 (0.1 mmol, 1 eq.) and triethylamine (26.4 μL, 0.15 mmol, 1.5 eq.) in dry DCM (1 mL). The reaction was stirred at 0° C. for 1 h, concentrated, and purified by flash column chromatography (silica gel, 0-100% EtOAc/hexanes gradient) to afford the title compound 9.

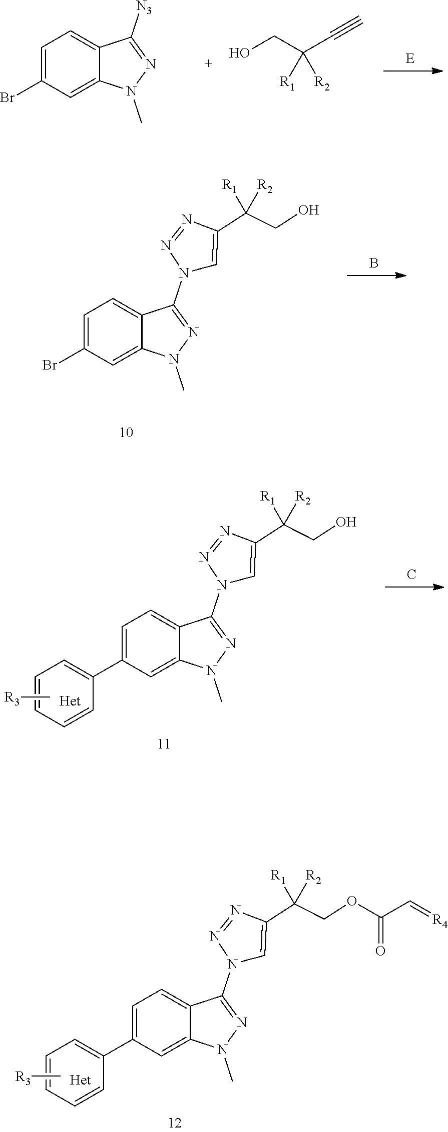

### General Synthetic Procedures

Procedure E: 3-Azido-6-bromo-1-methyl-1H-indazole (756 mg, 3 mmol) and alkyne alcohol (3 mmol) were suspended in 12 mL of a 1:1 water/tert-butanol mixture. Sodium ascorbate (0.3 mmol, 300 μL of freshly prepared 1 M solution in water) was added, followed by copper (II) sulfate pentahydrate (7.5 mg, 0.03 mmol, in 100 μL of water). The heterogeneous mixture was stirred vigorously overnight, at which point it cleared and TLC analysis indicated complete consumption of the reactants. The reaction mixture was diluted with 50 ml of water and cooled in ice, and the white precipitate was collected by filtration. After being washed with cold water (2×25 mL), the precipitate was dried under vacuum to afford product 10.

Procedure B: Compound 10 (0.1 mmol) and corresponding boronic acids (0.15 mmol) were placed in a 25 mL round-bottom flask, and toluene/ethanol (v/v=5:1) (3 mL) and sat. NaHCO₃ (3 mL) were added. The mixture was flushed by N₂ for 10 min. Then, Pd(dppf)Cl₂ (0.01 mmol) was added and the reaction mixture was allowed to react at 90° C. in an oil bath for 12 h. Then, the reaction mixture was cooled to room temperature and treated with H₂O and extracted with EtOAc. The combined organic layers were washed with brine and dried over anhydrous Na₂SO₄. After the removal of EtOAc, the residue was purified by column chromatography (silica gel, 0-100% EtOAc/hexanes gradient) to afford the product 11.

Procedure C: Acyl chloride (0.12 mmol, 1.2 eq.) was added dropwise to a stirred solution of compound 11 (0.1 mmol, 1 eq.) and triethylamine (26.4 μL, 0.15 mmol, 1.5 eq.) in dry DCM (1 mL). The reaction was stirred at 0° C. for 1 h, concentrated, and purified by flash column chromatography (silica gel, 0-100% EtOAc/hexanes gradient) to afford the title compound 12.

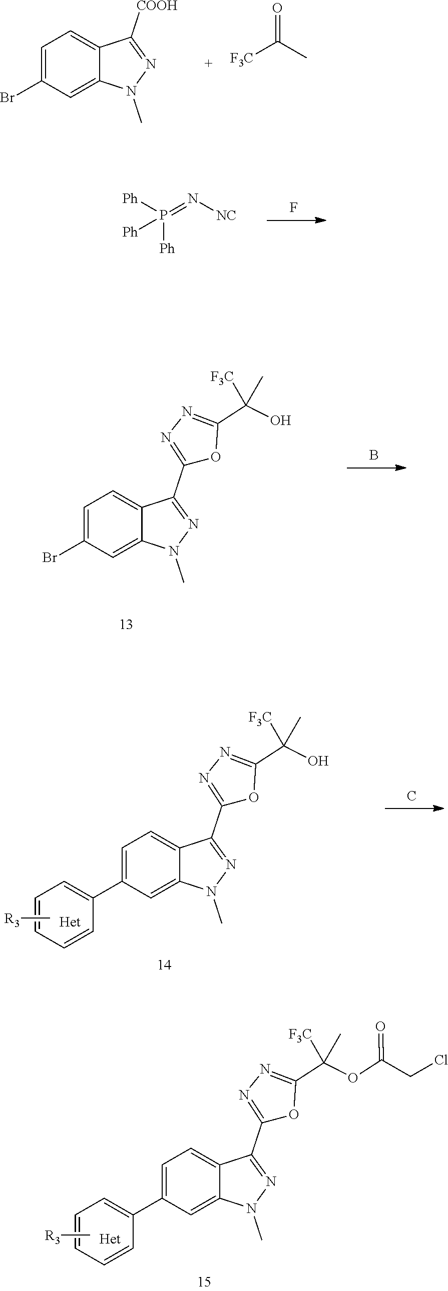

### General Synthetic Procedures

Procedure F: To a stirred solution of (N-isocyanimino) triphenylphosphorane (1 mmol) and 1,1,1-trifluoroacetone (1 mmol) in CH₂Cl₂ (10 mL) was added 6-bromo-1-methyl-1H-indazole-3-carboxylic acid (1 mmol) at room temperature. The mixture was stirred overnight. The solvent was removed under reduced pressure, and the viscous residue was purified by flash column chromatography (silica gel, 0-100% EtOAc/hexanes gradient). The solvent was removed under reduced pressure and the product 13 were obtained.

Procedure B: Compound 13 (0.1 mmol) and corresponding boronic acids (0.15 mmol) were placed in a 25 mL round-bottom flask, and toluene/ethanol (v/v=5:1) (3 mL) and sat. NaHCO₃ (3 mL) were added. The mixture was flushed by N₂ for 10 min. Then, Pd(dppf)Cl₂ (0.01 mmol) was added and the reaction mixture was allowed to react at 90° C. in an oil bath for 12 h. Then, the reaction mixture was cooled to room temperature and treated with H₂O and extracted with EtOAc. The combined organic layers were washed with brine and dried over anhydrous Na₂SO₄. After the removal of EtOAc, the residue was purified by column chromatography (silica gel, 0-100% EtOAc/hexanes gradient) to afford the product 14.

Procedure C: Chloroacetyl chloride (0.12 mmol, 1.2 eq.) was added dropwise to a stirred solution of compound 14 (0.1 mmol, 1 eq.) and triethylamine (26.4 μL, 0.15 mmol, 1.5 eq.) in dry DCM (1 mL). The reaction was stirred at 0° C. for 1 h, concentrated, and purified by flash column chromatography (silica gel, 0-100% EtOAc/hexanes gradient) to afford the title compound 15.

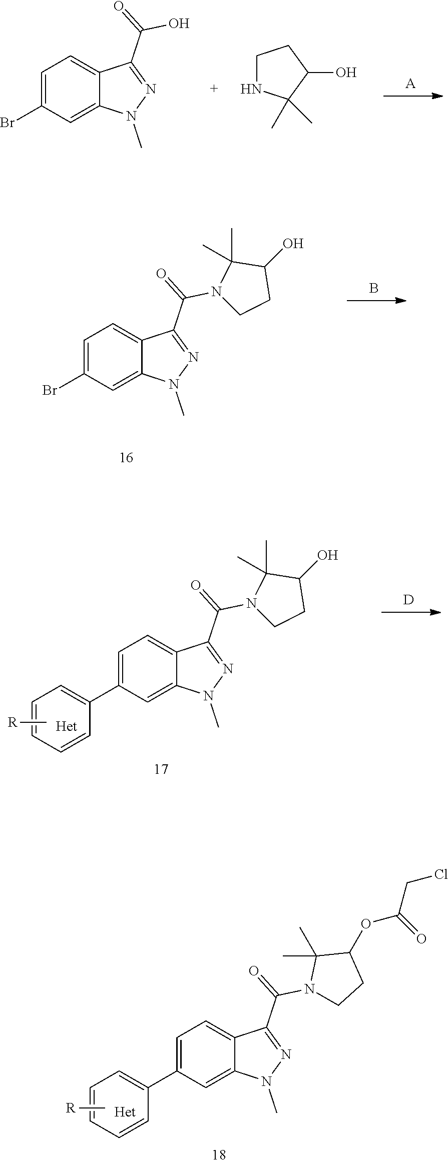

### General Synthetic Procedures

Procedure A: 2,2-Dimethylpyrrolidin-3-ol (3 mmol) was dissolved in anhyd. THF (12 mL) and then EDC (3 mmol), HOBt (3 mmol) and the corresponding 6-bromo-1-methyl-1H-indazole-3-carboxylic acid (2 mmol) were added sequentially at 25° C. After stirring overnight at r.t., evaporating the solvent, pouring the product into sat. aq NaHCO₃, extracting with EtOAc, washing with 1 N HCl, sat. aq NaHCO₃, brine, drying over Na₂SO₄ and concentrating the combined extract gave the amide 16.

Procedure B: Compound 16 (0.1 mmol) and corresponding boronic acids (0.15 mmol) were placed in a 25 mL round-bottom flask, and toluene/ethanol (v/v=5:1) (3 mL) and sat. NaHCO₃ (3 mL) were added. The mixture was flushed by N₂ for 10 min. Then, Pd(dppf)Cl₂ (0.01 mmol) was added and the reaction mixture was allowed to react at 90° C. in an oil bath for 12 h. Then, the reaction mixture was cooled to room temperature and treated with H₂O and extracted with EtOAc. The combined organic layers were washed with brine and dried over anhydrous Na₂SO₄. After the removal of EtOAc, the residue was purified by column chromatography (silica gel, 0-100% EtOAc/hexanes gradient) to afford the product 17.

Procedure C: Chloroacetyl chloride (9.5 μL, 0.12 mmol, 1.2 eq.) was added dropwise to a stirred solution of above mixture and triethylamine (26.4 μL, 0.15 mmol, 1.5 eq.) in dry DCM (1 mL). The reaction was stirred at 0° C. for 1 h, concentrated, and purified by flash column chromatography (silica gel, 0-100% EtOAc/hexanes gradient) to afford the title compound 18.

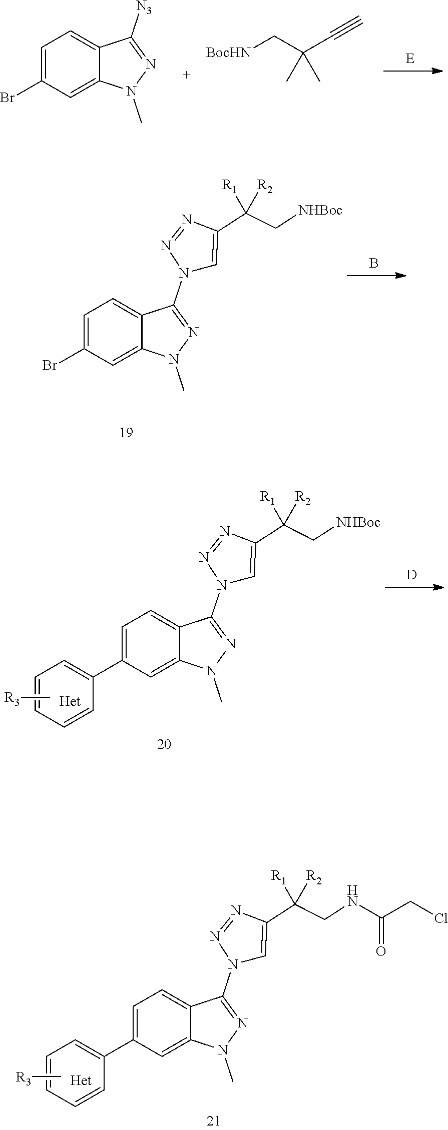

### General Synthetic Procedures

Procedure E: 3-Azido-6-bromo-1-methyl-1H-indazole (756 mg, 3 mmol) and tert-butyl (2,2-dimethylbut-3-yn-1-yl) carbamate (592 mg, 3 mmol) were suspended in 12 mL of a 1:1 water/tert-butanol mixture. Sodium ascorbate (0.3 mmol, 300 μL of freshly prepared 1 M solution in water) was added, followed by copper (II) sulfate pentahydrate (7.5 mg, 0.03 mmol, in 100 μL of water). The heterogeneous mixture was stirred vigorously overnight, at which point it cleared and TLC analysis indicated complete consumption of the reactants. The reaction mixture was diluted with 50 ml of water and cooled in ice, and the white precipitate was collected by filtration. After being washed with cold water (2×25 mL), the precipitate was dried under vacuum to afford product 19.

Procedure B: Compound 19 (0.1 mmol) and corresponding boronic acids (0.15 mmol) were placed in a 25 mL round-bottom flask, and toluene/ethanol (v/v=5:1) (3 mL) and sat. NaHCO₃ (3 mL) were added. The mixture was flushed by N₂ for 10 min. Then, Pd(dppf)Cl₂ (0.01 mmol) was added and the reaction mixture was allowed to react at 90° C. in an oil bath for 12 h. Then, the reaction mixture was cooled to room temperature and treated with H₂O and extracted with EtOAc. The combined organic layers were washed with brine and dried over anhydrous Na₂SO₄. After the removal of EtOAc, the residue was purified by column chromatography (silica gel, 0-100% EtOAc/hexanes gradient) to afford the product 20.

Procedure D: Trifluoroacetic acid (1 mmol, 10 eq.) was added to compound 20 (0.1 mmol) in dichloromethane (5 ml). The mixture was stirred at r.t. for 12 h and concentrated in vacuo for further use. Chloroacetyl chloride (9.5 μL, 0.12 mmol, 1.2 eq.) was added dropwise to a stirred solution of above mixture and triethylamine (26.4 μL, 0.15 mmol, 1.5 eq.) in dry DCM (1 mL). The reaction was stirred at 0° C. for 1 h, concentrated, and purified by flash column chromatography (silica gel, 0-100% EtOAc/hexanes gradient) to afford the title compound 21.

### Example 9

### Synthetic Procedures for RSL3 Analogs

This Example provides synthetic schemes for RSL3 analogs disclosed herein.

### General Synthetic Procedures

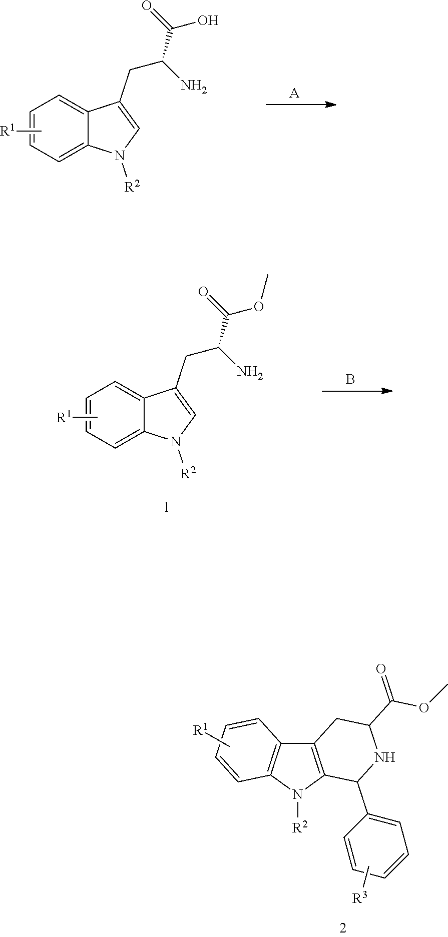

### Procedure A

To a solution of the corresponding substituted D-tryptophan (1.0 equiv) in methanol (20 equiv) was added thionyl chloride (1.2 equiv) over 10 min at 0° C. The resulting mixture was allowed to come to room temperature and then was heated in reflux overnight. Upon reaction completion, the reaction mixture was cooled to room temperature and the solvent was removed under reduced pressure. The resulting crude was extracted with aq. solution of sodium bicarbonate (NaHCO₃) and ethyl acetate. The combined organic layers were washed with brine, dried over magnesium sulfate (MgSO4), and concentrated in vacuum. The compounds were used without further purification for the following reaction.

### Procedure B

To a suspension of the corresponding substituted D-tryptophan methyl ester 1 (1.2 equiv) in dichloromethane (DCM) were added the corresponding aldehyde (1.0 equiv) and trifluoroacetic acid (TFA) (3.1 equiv) and the solution was refluxed for 12 hours. Upon reaction completion, the reaction mixture was cooled to room temperature and quenched with 30% aq. solution NaOH until PH˜7. The phases were separated, and the resulting aqueous phase was extracted with dichloromethane. The combined organic layers were washed with water and brine, dried over magnesium sulfate (MgSO₄), and concentrated in vacuum. The crude reaction mixture was purified by means of short silica-gel column chromatography (eluent system: hexanes-ethyl acetate) to afford compound 2 as two separate diastereomers.

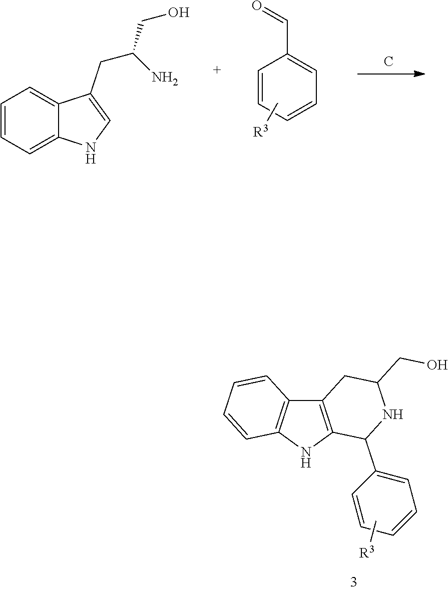

### Procedure C

To a suspension of D-tryptophanol (1.2 equiv) in a 4:1 DCM/MeOH mixture were added the corresponding aldehyde (1.0 equiv) and trifluoroacetic acid (TFA) (3.1 equiv) and the solution was refluxed for 12 hours. Upon reaction completion, the reaction mixture was cooled to room temperature and quenched with 30% aq. solution NaOH until PH˜7. The phases were separated, and the resulting aqueous phase was extracted with dichloromethane. The combined organic layers were washed with water and brine, dried over magnesium sulfate (MgSO4), and concentrated in vacuum. The crude reaction mixture was purified by means of short silica-gel column chromatography (eluent system: DCM-MeOH) to afford compound 3 as two separate diastereomers.

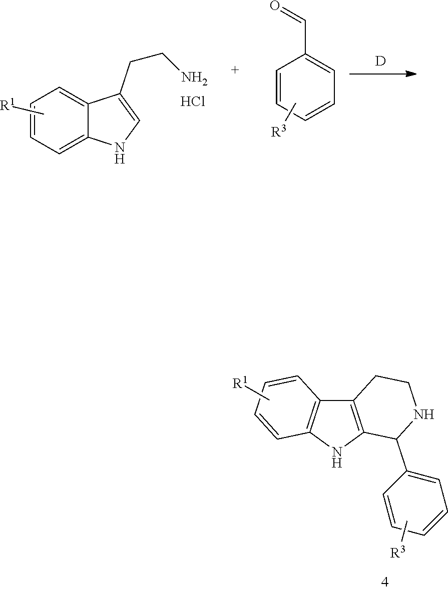

### Procedure D

A suspension of D-tryptamine hydrochloride and the corresponding aldehyde in glacial acetic acid was heated at 80° C. overnight. The resulting precipitate was collected by filtration, rinsed with hexanes and dried briefly. The solid was then stirred with dichloromethane and 10% aq. solution sodium carbonate until all the solids were dissolved. The layers were separated and the aqueous layer was washed again with dichloromethane. The combined organic layers were washed with brine, dried over magnesium sulfate (MgSO4) and the solvent was concentrated under reduced pressure. The compounds were used without further purification for the following reaction.

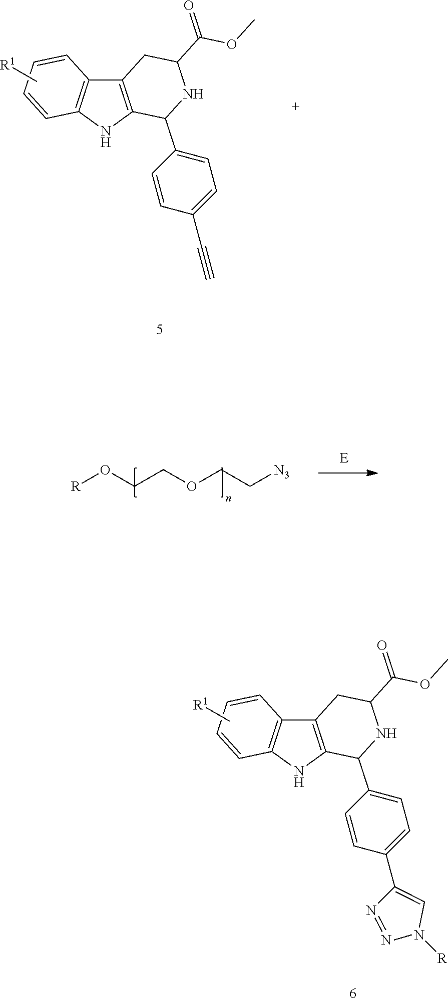

### Procedure E

Compound 5 and the corresponding PEG azide (1 mmol) were dissolved in a 1:1 water/tert-butanol mixture. Sodium ascorbate (5 mol %) was added, followed by copper (II) sulfate pentahydrate (2 mol %). The reaction mixture was stirred vigorously overnight at room temperature. When TLC analysis indicated complete consumption of the reactants, the reaction mixture was extracted with ethyl acetate. The combined organic layers were washed with water and brine, dried over magnesium sulfate (MgSO4), and concentrated in vacuum. The crude reaction mixture was purified by means of short silica-gel column chromatography (eluent system: dichloromethane-methanol) to afford compound 6.

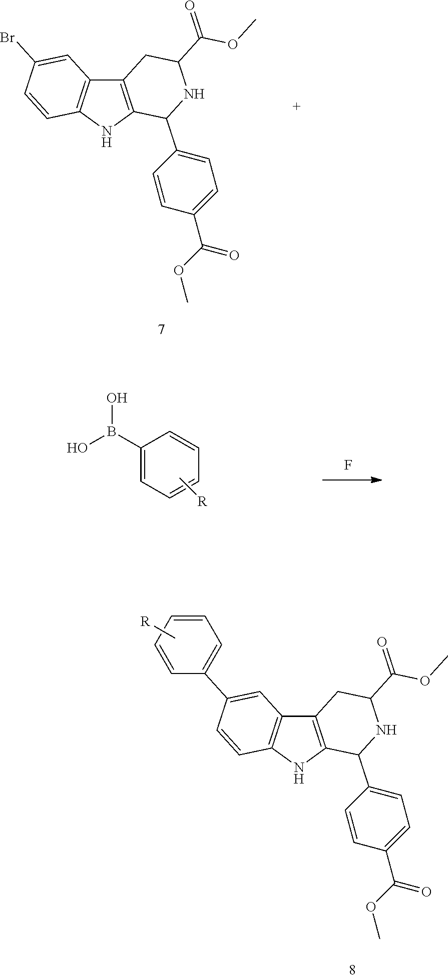

### Procedure F

Compound 7 (1.0 equiv) and the corresponding boronic acids (1.5 equiv) were placed in a 50 mL round-bottom flask, and toluene/ethanol (v/v=5:1) and sat. NaHCO₃ (1.5 equiv) were added. The mixture was flushed by N₂ for 10 min. Then, Pd(dppf)Cl₂ (1 mol %) was added and the reaction mixture was allowed to react at 90° C. in an oil bath for 12 h. Then, the reaction mixture was cooled to room temperature and treated with H₂O and extracted with EtOAc. The combined organic layers were washed with brine and dried over anhydrous MgSO₄. After the removal of EtOAc, the residue was purified by flash column chromatography (eluent system: hexanes-ethyl acetate) to afford compound 8.

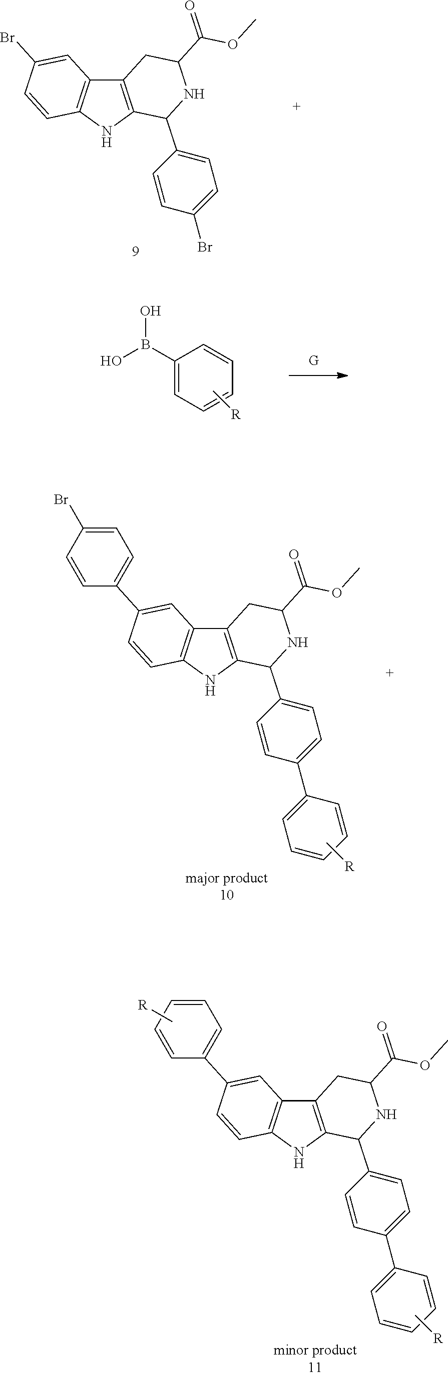

### Procedure G

Compound 9 (1.0 equiv) and the corresponding boronic acids (1.1 equiv) were placed in a 50 mL round-bottom flask, and toluene/ethanol (v/v=5:1) and sat. NaHCO₃ (1.5 equiv) were added. The mixture was flushed by N₂ for 10 min. Then, Pd(dppf)Cl₂ (1 mol %) was added and the reaction mixture was allowed to react at 90° C. in an oil bath for 12 h. Then, the reaction mixture was cooled to room temperature and treated with H₂O and extracted with EtOAc. The combined organic layers were washed with brine and dried over anhydrous MgSO4. After the removal of EtOAc, the residue was purified by flash column chromatography (eluent system: hexanes-ethyl acetate) to afford compound 10 as the major product and compound 11 as minor product.

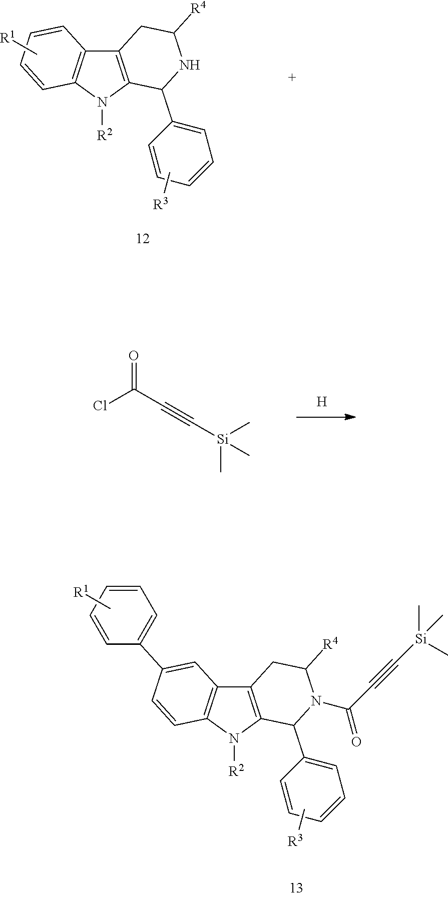

### Procedure H

An oven-dried 25 mL round-bottom flask equipped with a magnetic stirring bar was charged with compound 12 (1.0 equiv) in dry dichloromethane and triethylamine (Et₃N) (2.5 equiv). The mixture was cooled in an ice bath to 0° C. and 3-(trimethylsilyl) propioloyl chloride 95% (2.3 equiv) was added. The resulting solution was warmed to room temperature and stirred overnight. Upon reaction completion, the reaction mixture was concentrated under reduced pressure and the crude residue was purified by flash column chromatography on silica gel (eluent system: hexanes-ethyl acetate) to afford compound 13.

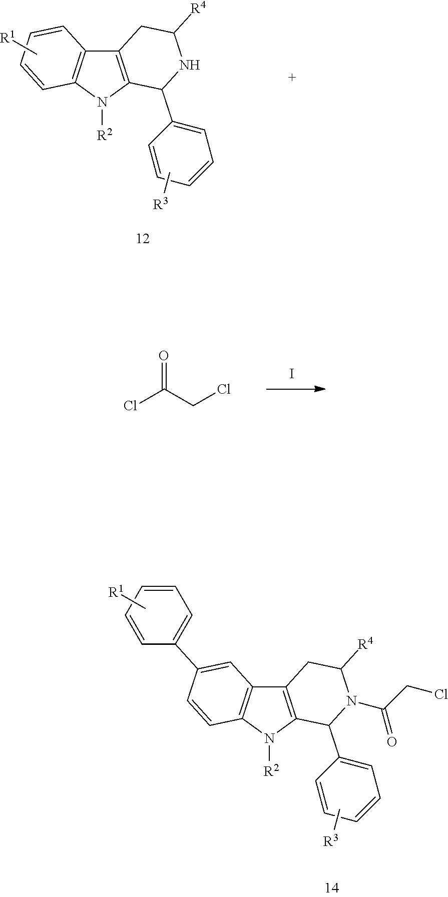

### Procedure I

An oven-dried 25 mL round-bottom flask equipped with a magnetic stirring bar was charged with compound 12 (1.0 equiv) in dry dichloromethane and triethylamine (Et₃N) (2.5 equiv). The mixture was cooled in an ice bath to 0° C. and chloroacetyl chloride (2.3 equiv) was added. The resulting solution was warmed to room temperature and stirred overnight. Upon reaction completion, the reaction mixture was concentrated under reduced pressure and the crude residue was purified by flash column chromatography on silica gel (eluent system: hexanes-ethyl acetate) to afford compound 14.

### Example 10

### Novel Potent and Selective GPX4 Inhibitors Via Structure-Based Drug Design

### Summary

Glutathione Peroxidase 4 (GPX4) is an attractive therapeutic target for drug-resistant and metastatic cancers, but successful targeting of GPX4 has proved to be difficult. Recently, using screening, we found LOC1886 as a hit compound for GPX4 inhibition. Based on the structure-guided drug design approach, we identified a number of structurally diverse lead compounds that inhibit GPX4. After evaluation of their inhibitory activity, cellular potency, metabolic stability, and water solubility, we identified several metabolic weak spots which could potentially be blocked for improved ADMET properties. We also identified multiple lead compounds with ˜40 nM potency for inhibiting GPX4, determined that they have selectivity >1000, the ability to induce ferroptosis in persister cells and obtained a co-crystal structure of the GPX4^{U46C} in complex with QW-314. We selected one compound, termed QW-594, as an optimized lead and characterized it in pharmacokinetic study in vivo. QW-852 is not only a powerful research tool with which to investigate the biology of GPX4 and the therapeutic potential of selective GPX4 protein depletion and inhibition but also a promising lead compound toward ultimate development of a GPX4-targeted therapy.

### Introduction

Ferroptosis is a form of nonapoptotic regulated cell death characterized by iron-dependent lipid peroxidation (Dixon et al., 2012). Since its discovery in 2012, activation of ferroptosis has emerged as a promising anti-cancer strategy that has been demonstrated to contribute to the therapeutic benefit of and synergize with a number of existing anti-neoplastic treatment regimens (EMMA LACHAIER and MAXIME BAERT, 2014; Liangyu Chen, 2015; Lo et al., 2008; Roh et al., 2016; Wang et al., 2019; Ye et al., 2020; Yu et al., 2015; Zou et al., 2019). Glutathione peroxidase 4 (GPX4) is a key negative regulator of the ferroptosis pathway that detoxifies the phospholipid hydroperoxides that accumulate within cell membranes and drive ferroptotic cell death (Yang et al., 2014). Recently, therapy-resistant cancer cell states, including cells that have undergone epithelial-to-mesenchymal transition (EMT) during the course of metastasis as well as quiescent drug-tolerant persister cells implicated in tumor relapse, have been shown to be exquisitely dependent on GPX4 for redox homeostasis and cell survival (Hangauer et al., 2017; Viswanathan et al., 2017). As such, GPX4 represents a promising target for therapeutic intervention.

GPX4 is one of eight human glutathione peroxidases (GPXs) and possesses a conserved catalytic triad consisting of selenocysteine 46 (U46), glutamine 81 (Q81), and tryptophan 136 (W136). Based on structural alignments with the other GPXs, GPX4 is unique in that it possesses a relatively exposed active site localized at a flat impression of the protein surface, enabling GPX4 to act on various complex lipid substrates (Moosmayer et al., 2021; Patrick Scheerer, 2007). However, the absence of an obvious small molecule binding pocket likely necessitates a covalent mechanism reliant on the catalytic activity of the selenocysteine residue. Indeed, all existing GPX4 inhibitors covalently target the active site selenocysteine using an electrophilic warhead, such as a chloroacetamide, masked nitrile oxide, or a propiolamide moiety (Eaton et al., 2020a; Eaton et al., 2020b; Eaton et al., 2019; Xu et al., 2021; Yang and Stockwell, 2008). Of these, chloroacetamide-containing compounds, such as RSL3, were the first GPX4 inhibitors to be discovered and have remained amongst the most potent inducers of ferroptosis in many cellular contexts. However, most current inhibitors are limited by several factors, including poor selectivity, aqueous solubility, metabolic stability, and/or pharmacokinetic properties. These limitations complicate the use of existing inhibitors as tool compounds for the interrogation of ferroptosis in physiological and disease states as well as hinder their potential in the development of GPX4-targeted therapeutics. As such, it is necessary to expand the current pharmacological repertoire of GPX4 inhibitors and deepen our understanding of the structural basis of small molecule binding and inhibition of GPX4.

Here, we report the development of a novel GPX4 inhibitor from LOC1886, a high-throughput screening hit we described previously. Using structure-based drug design and iterative rounds of structure-activity relationship studies, we produced four generations of structurally-distinct analogs, ultimately yielding QW-852 as our most potent and specific compound to date. We demonstrate that QW-852 is comparably potent to RSL3 and has improved ferroptosis specificity in cells. Further, we show that QW-852 preferentially induces ferroptosis in a drug-tolerant persister cell paradigm of post-chemotherapeutic disease relapse.

### Synthesis of Compounds

Unless noted, all the following reactions were performed in flame or oven-dried glassware and carried out under an atmosphere of argon or nitrogen with magnetic stirring, unless otherwise stated. Anhydrous solvents were used as supplied (Acros Organics ExtraDry or over molecular sieves). N,N-Diisopropylamine was purified by distillation from potassium hydroxide under argon. All other reagents were used as supplied unless otherwise stated (Aldrich, Chem-Impex, TCI America, etc). Thin layer chromatography was performed on SiliCycle® 250 μm 60 A plates or Merck Kieselgel 60 F254 0.20 mm precoated, glass backed silica gel plates. Visualization was accomplished with 254 nm UV light, KMnO4 stain, and/or by p-anisaldehyde staining solution. All column chromatography was performed using general flash techniques on SiliCycle® SilicaFlash® P60, 40-63 μm 60 A. For particularly difficult separations, automated normal phase flash column chromatography was carried out on Teledyne Isco Combiflash RF Plus using CombiFlash gold pre-packed columns with UV/ELS detector using HPLC grade solvent (Fisher Scientific) with the indicated solvent system.

NMR spectra were recorded on Bruker AV III 400 or AV III 500 MHz spectrometers at ambient temperature. Chemical shifts (δ) for 1H NMR spectra are reported in parts per million (ppm) from Me4Si with the solvent resonance as the internal standard (CDCl3=7.26 ppm, CD3OD=3.31 ppm) with multiplicity (s=singlet, bs=broad singlet, d=doublet, t=triplet, q=quartet, and m=multiplet) and coupling constants (in Hz). ¹³C NMR spectra are reported in ppm from Me4Si with the solvent resonance as the internal standard (CDCl3=77.16 ppm, CD3OD=49.00 ppm). ¹⁹F NMR spectra are reported in ppm from CDCl₃ and are uncorrected. High Resolution Mass spectrometry (HRMS) was carried out at the Mass Spectrometry Facility at the Chemistry Department of Columbia University in the City of New York and recorded on a Waters Acquity H UPLC-MS.

Synthesis pathways for selected compounds are shown below:

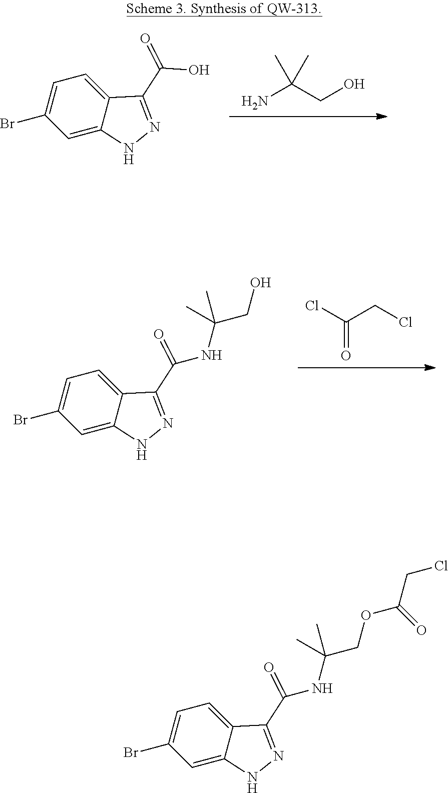

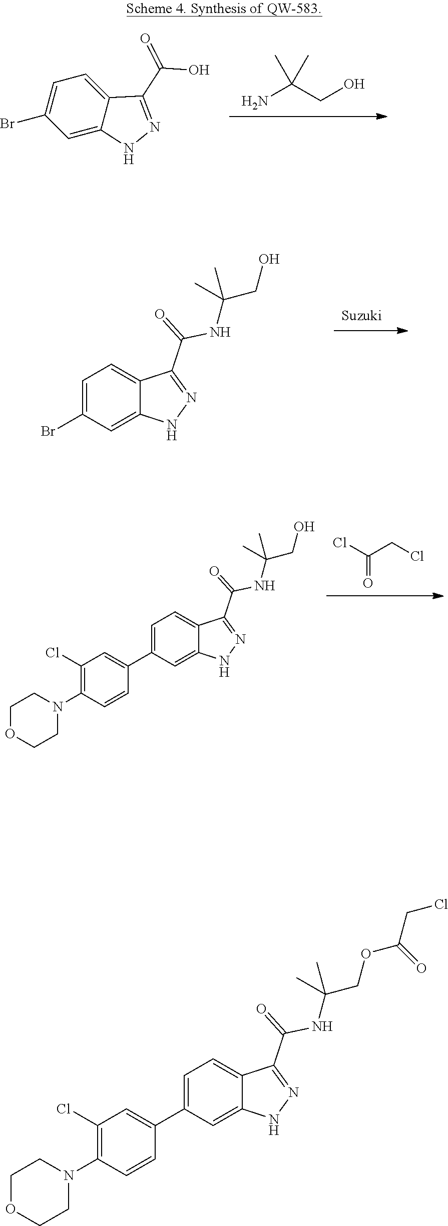

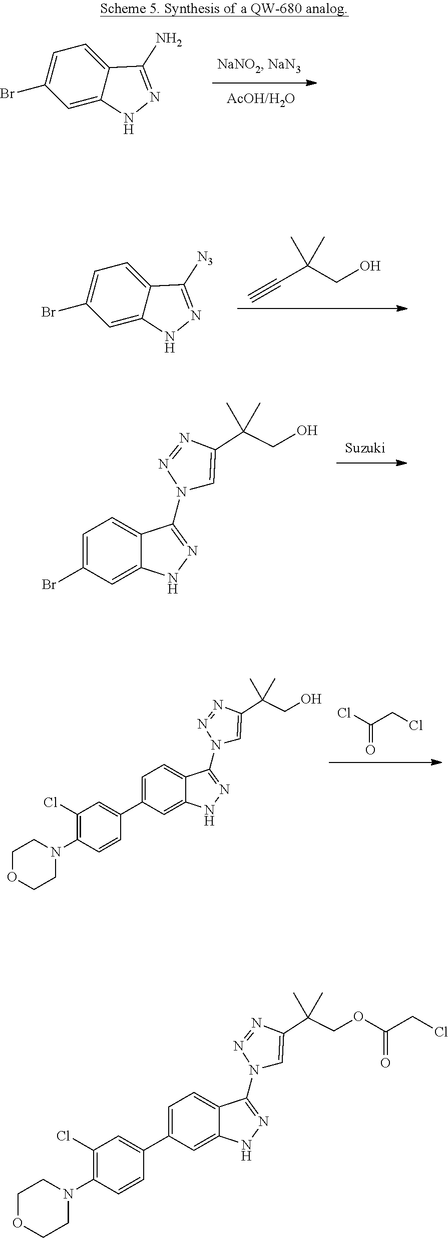

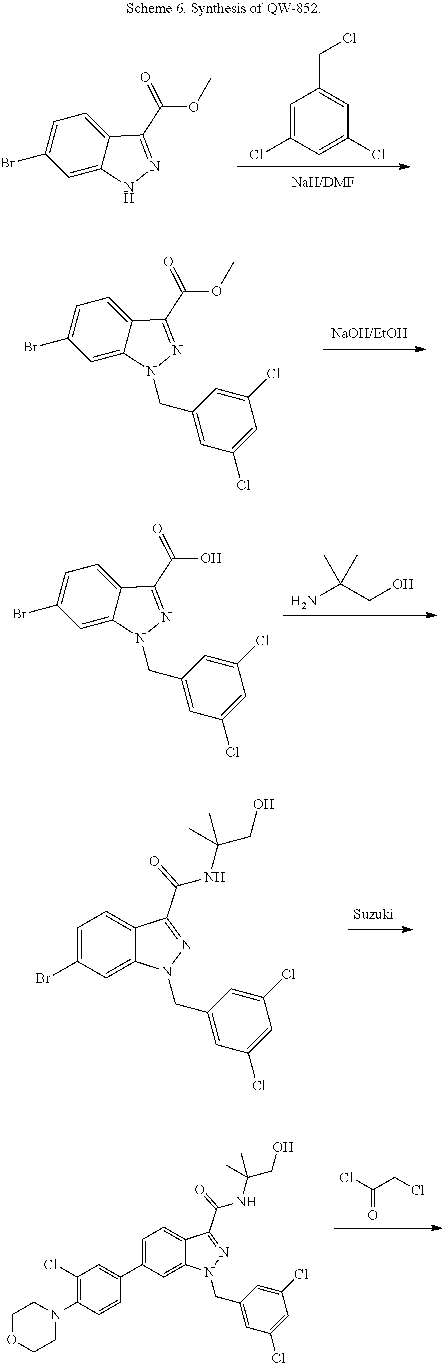

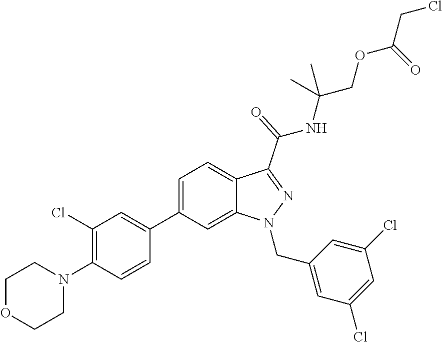 [SMILES: CC(C)(COC(=O)CCl)NC(=O)c1nn(Cc2cc(Cl)cc(Cl)c2)c2cc(-c3ccc(N4CCOCC4)c(Cl)c3)ccc12]

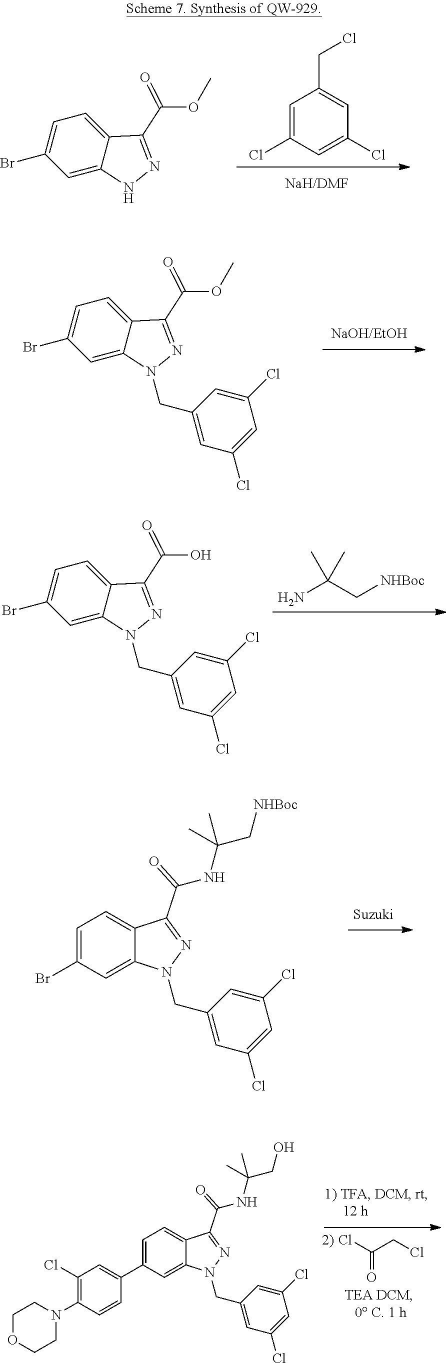

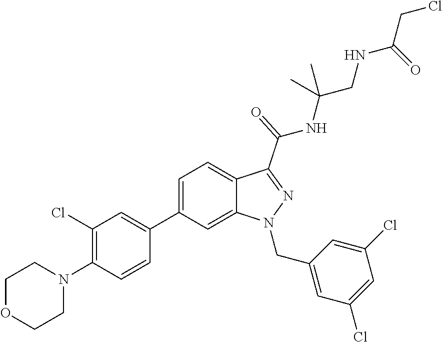 [SMILES: CC(C)(CNC(=O)CCl)NC(=O)c1nn(Cc2cc(Cl)cc(Cl)c2)c2cc(-c3ccc(N4CCOCC4)c(Cl)c3)ccc12]

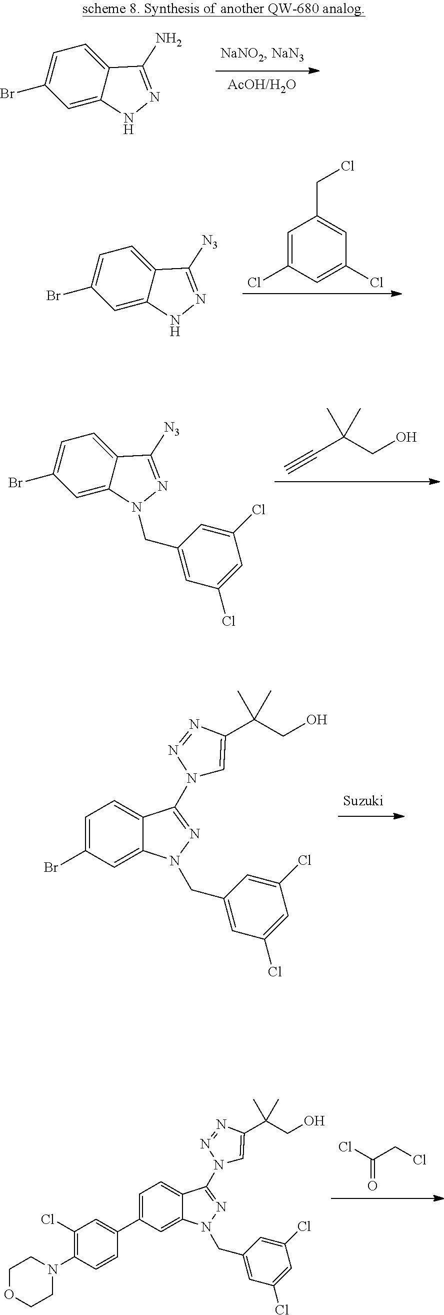

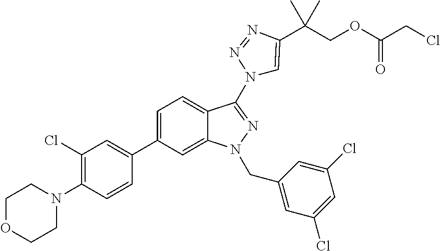 [SMILES: CC(C)(COC(=O)CCl)c1cn(-c2nn(Cc3cc(Cl)cc(Cl)c3)c3cc(-c4ccc(N5CCOCC5)c(Cl)c4)ccc23)nn1]

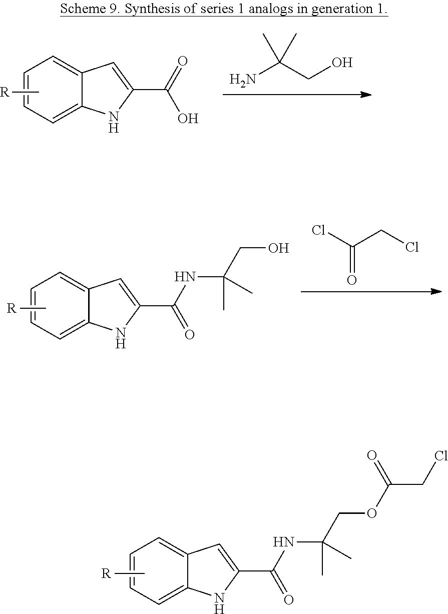

### Results

### LOC1886 has Low Potency and Shows Non-Specificity of Ferroptosis

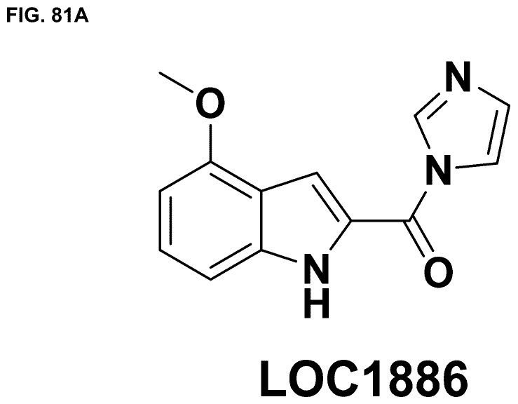

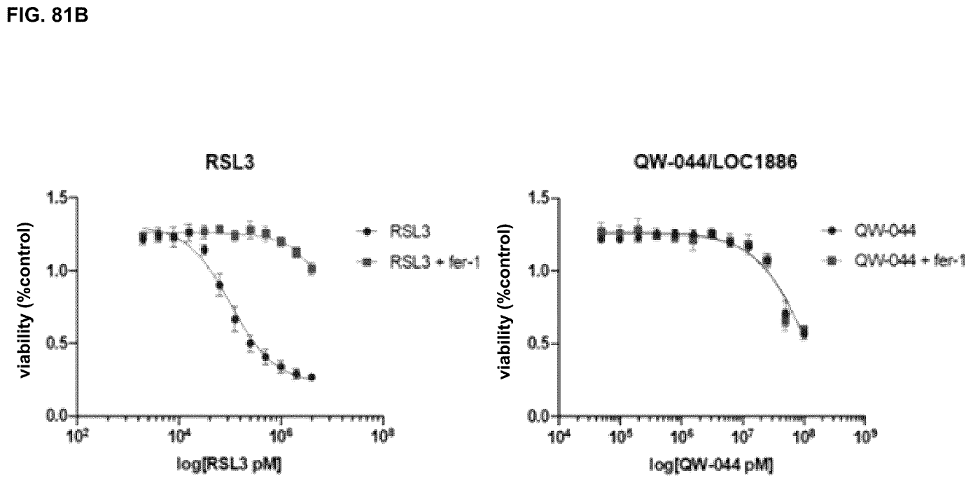

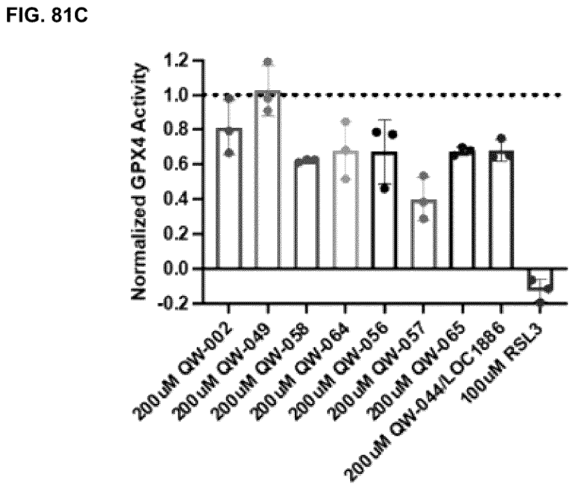

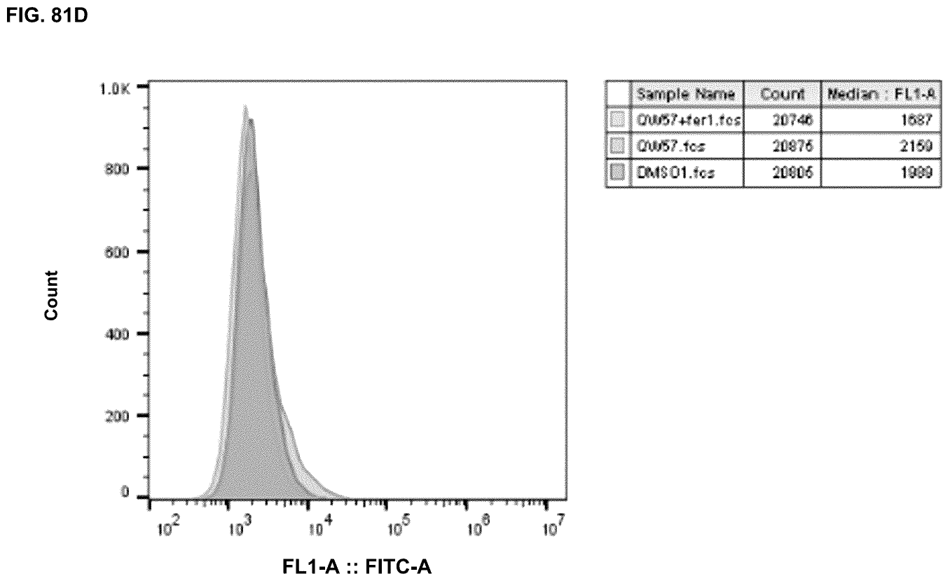

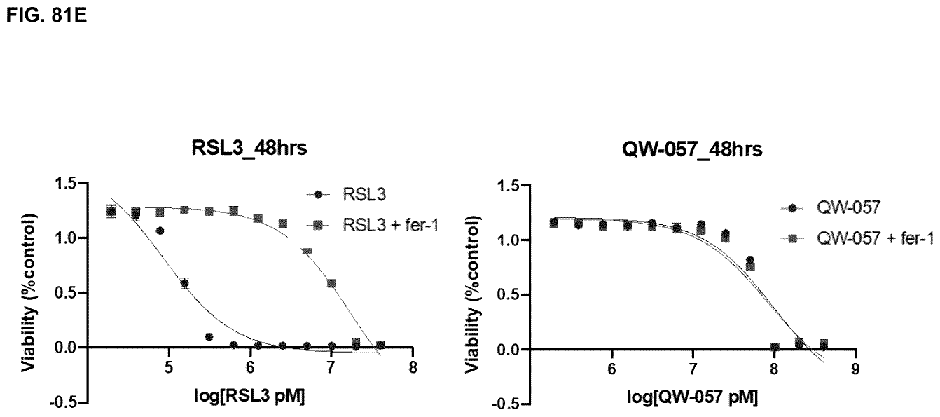

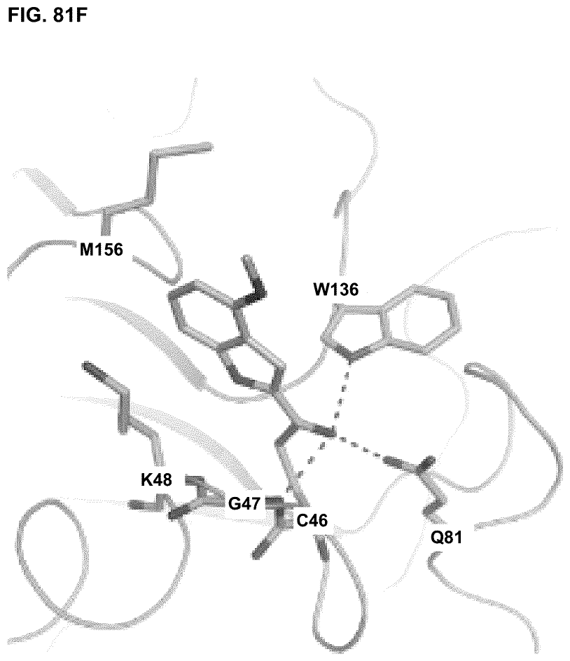

To date, some GPX4 inhibitors have been reported, but none of them are suitable for development as drugs; hence, new scaffolds are needed. We recently identified a hit compound LOC1886 (FIG. 81A) by the high-throughput screening of a lead-optimized compound (LOC) library as small molecule binder of GPX4^{U46C}. LOC1886 inhibits GPX4 activity in vitro, as well as degrades GPX4 protein and induces fer-1 rescuable increases in lipid peroxidation in cells. Co-crystal structure of GPX4^{U46C} with LOC1886 reveals that LOC1886 bound to allosteric sites C66 and C10 of GPX4^{U46C}. Even LOC1886 showed some characteristics of a GPX4 inhibitor, however, cellular dose-response assays showed that LOC1886 induced other cell death modalities as well since no fer-1 rescue was observed (FIG. 81B), suggesting further optimization was necessary to obtain more potent and selective GPX4 inhibitors for specifically inducing ferroptosis in cells.

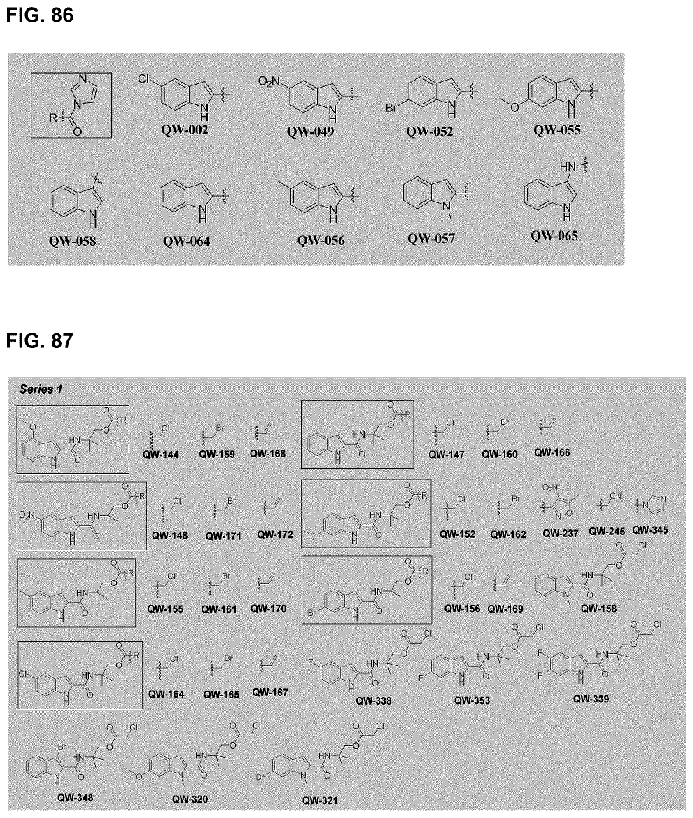

To improve the potency and specificity of LOC1886 for ferroptosis induction, we synthesized and assayed an initial library of analogs maintaining the imidazole ring but varying the indole moiety which were chosen to be diverse in substitution patterns and electronic effects (FIG. 86). The substituted indoles bear substituents that range from electron-withdrawing to highly electron-donating substituents, which we expected to have an influence on the reactivity. We also chose to link the electrophile to indole via an amide linker (QW-065) because of their modulating influence on the reactivity of the adjacent electrophile. Assessment of their in vitro GPX4 inhibitory activities showed that only the analog with a methyl group on the N-site of the indole ring (QW-057) provided a nearly 2-fold increase in the GPX4 inhibitory activity at 200 uM concentration in comparison to LOC1886 (FIG. 81C, Table 2). QW-057 also had better activity than LOC1886 in the HT1080 cell viability assay (Table 2). Nevertheless, QW-057 did not induce lipid peroxidation and the potency of QW-057 against GPX4 is still insufficient and should be structurally optimized (FIG. 81D). Similarly, no fer-1 rescue was observed in the cellular dose-response assays of QW-057 (FIG. 81E).

TABLE 2
GPX4 activity and cellular activity assay data
for LOC1886 analogs (Related to FIG. 86).

| | GPX4% | | | | |
|---|---|---|---|---|---|
| | inhibition at | IC₅₀ (μM) | | IC₅₀ (μM) | |
| Compound | 200 (μM) | HT1080^{a} | HT1080^{b} | HEPG2^{a} | HEPG2^{b} |
| RSL3 | 112.5 ± 6.5 | 0.044 | 4.6 | 5.4 | 32.2 |
| LOC1886 | 31.9 ± 6.0 | 163.2 | 109.3 | 88.6 | 29.1 |
| QW-002 | 18.7 ± 15.8 | NT | NT | NT | NT |
| QW-049 | −2.6 ± 14.6 | NT | NT | NT | NT |
| QW-058 | 37.8 ± 0.9 | NT | NT | NT | NT |
| QW-064 | 31.9 ± 16.6 | NT | NT | NT | NT |
| QW-056 | 32.7 ± 18.4 | NT | NT | NT | NT |
| QW-057 | 59.8 ± 12.5 | 64.0 | 64.2 | 97.7 | 82.6 |
| QW-065 | 32.4 ± 2.4 | >400 | >400 | >400 | >400 |

Data are expressed as the mean ± s.d. of each compound, n = 3 technical replicates.
^{a}HT1080 incubated with compounds alone.
^{b}HT1080 incubated with compounds and fer-1 1.0 μM.
^{c}HT1080^{a} IC₅₀/HT1080^{b} IC₅₀.

Examination of the protein structure around C66 showed a lack of suitable ligand binding pockets making the design of small molecule inhibitors targeting this site challenging. Because the ¹H, ¹⁵N-HSQC-NMR and co-crystal structures utilized mutant GPX4^{U46C} and LOC1886 operates via a covalent mechanism, we hypothesized that LOC1886 may also target the wildtype active site since the native selenocysteine is a much stronger nucleophile than other surface-exposed cysteines. As such, we conducted a molecular modeling of LOC1886 targeting the active site (FIG. 81F).

### LOC1886 was Converted to a New Scaffold as Generation 1

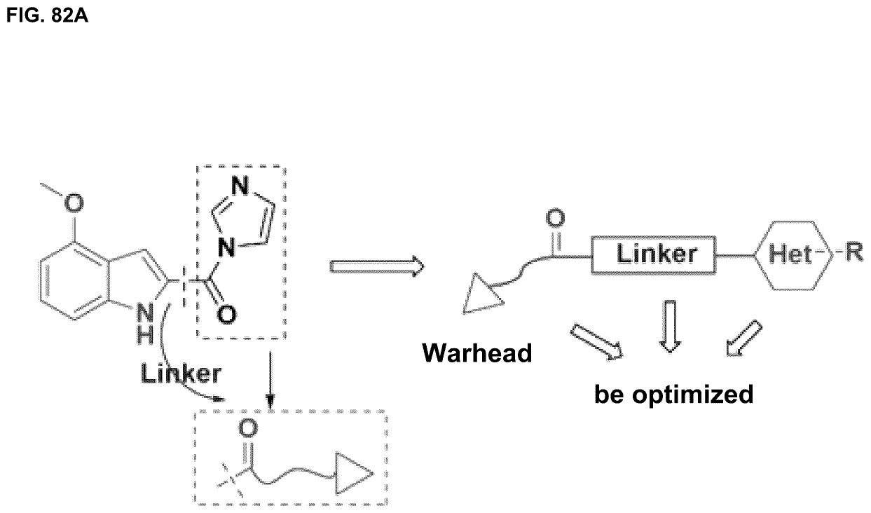

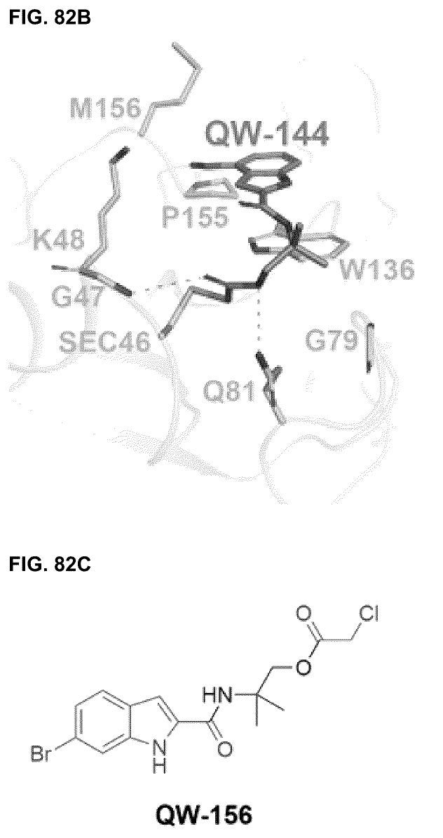

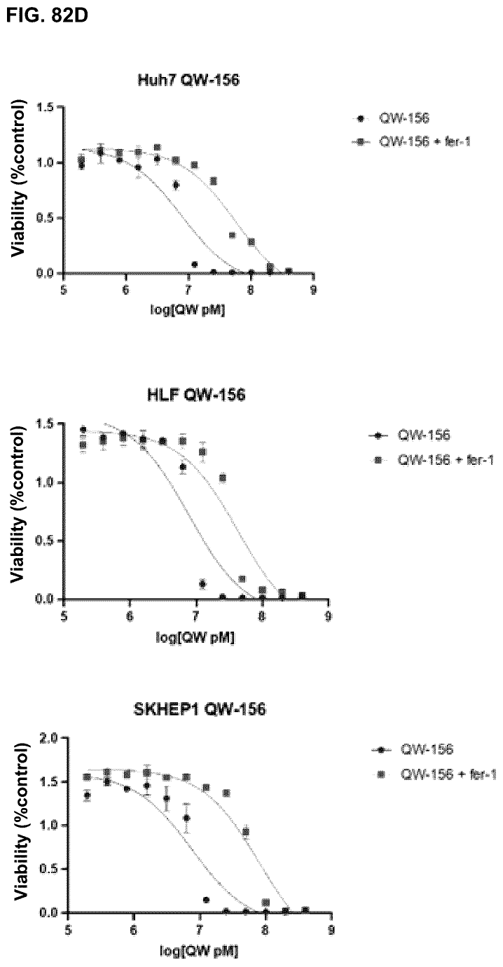

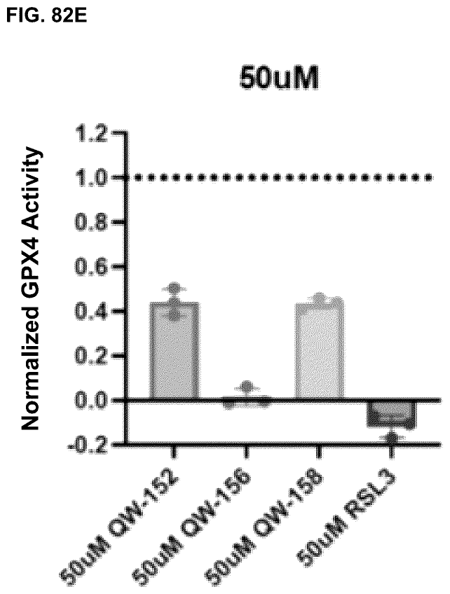

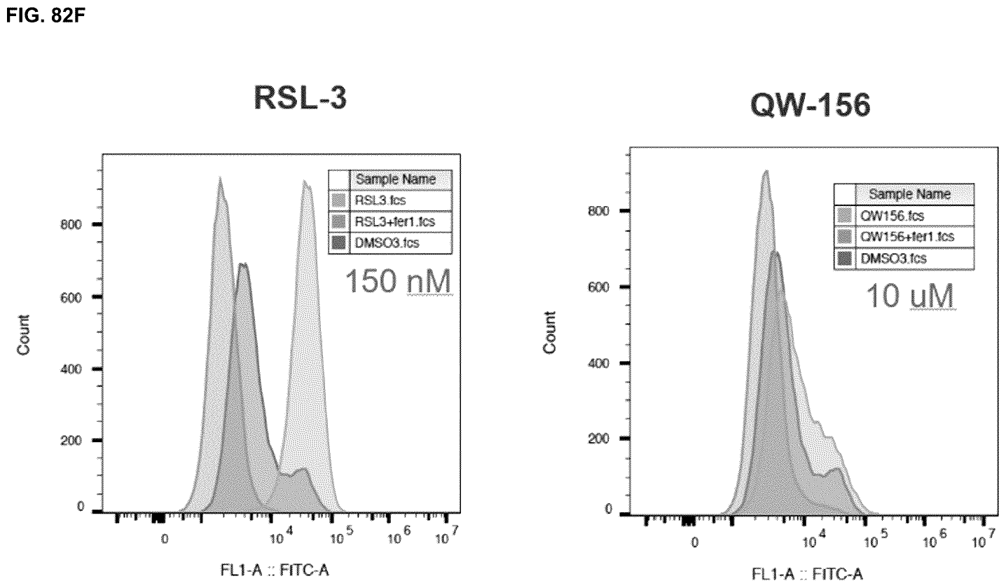

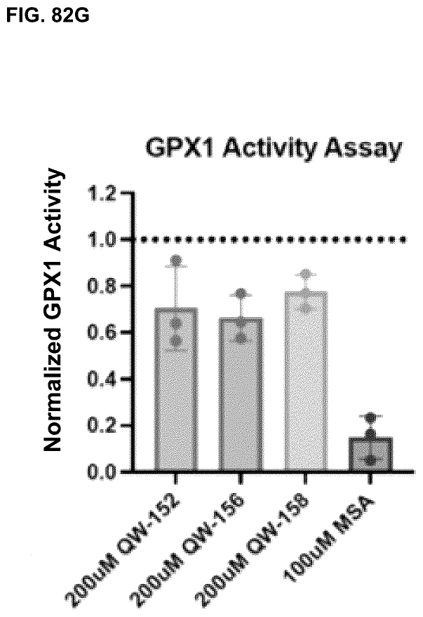

As no significant improvement in potency can be obtained by simply modulating substituted groups or the linker of LOC1886, scaffold hopping was necessary for the development of LOC1886 to new class of GPX4 inhibitor (FIG. 82A). First, we explored replacing the imidazole with stronger electrophiles. Since a variety of electrophiles containing the warheads of existing GPX4 inhibitors RSL3 and ML210, were expected to be slightly more reactive alternatives to the imidazoamide, we introduced and evaluated the activities of these electrophiles on the LOC1886 scaffold. Additionally, based on molecular modeling using the crystal structure of GPX4^{U46C} in complex with LOC1886 at the active site, we also incorporated a slightly longer, rigid linker to facilitate IT-IT stacking of the indole ring with tryptophan 136 (W136) (FIG. 82B). Therefore, a series of compounds in new scaffold was synthesized by coupling substituted indole-2-carboxylic acids with the 2-amino-2-methyl-1-propanol and then reacted with corresponding acid chlorides to introduce diverse electrophiles (FIG. 87). In general, analogs with the chloroacetyl warhead but varied the substituents in indoles displayed moderate to good GPX4 inhibitory activities. Of these, compounds QW-152, QW-156, QW-158 bearing 6-methoxy, 6-bromo and N, 1-methyl in the indole ring, respectively, showed complete inhibition at 200 μM concentration (FIG. 87, Table 3). However, excising the substitutents in the indole ring altogether (QW-147) led to a complete loss in potency of inhibitory activity even in the presence of chloromethyl ketone, suggesting that the chloroacetyl group does not confer GPX4 inhibitory activity nonspecifically (Table 3). Additionally, various electrophiles besides the chloroacetyl group were evaluated in this series and only bromomethyl ketone was tolerated from a potency standpoint (FIG. 87, Table 3). In series 2, we prepared indole-3-carboxamides as the novel analogs and explored extended linker lengths on either side of the amide bond, but these compounds generally showed diminished activity compared to QW-156 (FIG. 88, Table 3). It is worth noting that indole-3-carboxamide QW-280 exhibited significantly improved inhibitory activity compared to indole-2-carboxamide QW-417, which was inactive during GPX4 inhibition. Besides, alternative aromatic heterocycles replacing the indole ring were also assayed, including thiophene, benzothiophene, and pyrrole groups, again with the previously mentioned electrophiles. These compounds exhibited a loss of GPX4 inhibitory activity in vitro (FIG. 88, Table 3).

TABLE 3
GPX4 inhibition data for structural analogs of generation 1.

| | GPX4% inhibition at | | | |
|---|---|---|---|---|
| Compound | 200 (μM) | 100 (μM) | 50 (μM) | 20 (μM) |

Series 1

| | | | | |
|---|---|---|---|---|
| QW-144 | 50.0 ± 32.5 | NT | NT | NT |
| QW-159 | 56.0 ± 7.3 | NT | NT | NT |
| QW-168 | −8.8 ± 10.8 | NT | NT | NT |
| QW-147 | −3.3 ± 45.3 | NT | NT | NT |
| QW-160 | 33.3 ± 6.0 | NT | NT | NT |
| QW-166 | 1.8 ± 8.0 | NT | NT | NT |
| QW-148 | 87.3 ± 34.6 | 62.7 ± 10.9 | 79.2 ± 5.8 | 66.7 ± 1.4 |
| QW-171 | 83.9 ± 16.8 | NT | NT | NT |
| QW-172 | 16.1 ± 16.0 | NT | NT | NT |
| QW-152 | 150.4 ± 20.3 | 70.5 ± 9.2 | 56.0 ± 6.2 | 27.9 ± 5.5 |
| QW-162 | 103.8 ± 7.6 | NT | NT | NT |
| QW-237 | 2.0 ± 19.2 | NT | NT | NT |
| QW-245 | −9.6 ± 17.5 | NT | NT | NT |
| QW-345 | −7.4 ± 6.4 | NT | NT | NT |
| QW-155 | 50.9 ± 3.6 | NT | NT | NT |
| QW-161 | 65.0 ± 7.2 | NT | NT | NT |
| QW-170 | 5.3 ± 10.0 | NT | NT | NT |
| QW-156 | 170.3 ± 33.5 | 95.5 ± 6.2 | 98.6 ± 4.1 | 61.2 ± 0.5 |
| QW-169 | 14.0 ± 8.6 | NT | NT | NT |
| QW-158 | 133.2 ± 35.6 | 73.0 ± 2.4 | 56.5 ± 2.5 | 29.1 ± 10.0 |
| QW-164 | 53.3 ± 37.8 | NT | NT | NT |
| QW-165 | 73.9 ± 5.3 | NT | NT | NT |
| QW-167 | 7.8 ± 12.8 | NT | NT | NT |
| QW-338 | 42.6 ± 7.0 | NT | NT | NT |
| QW-353 | 69.6 ± 6.2 | NT | NT | NT |
| QW-339 | 43.4 ± 11.5 | NT | NT | NT |
| QW-348 | 99.6 ± 3.2 | NT | NT | NT |
| QW-320 | 84.3 ± 9.3 | NT | NT | NT |
| QW-321 | 90.3 ± 4.8 | NT | NT | NT |

Series 2

| | | | | |
|---|---|---|---|---|
| QW-280 | 75.9 ± 15.5 | NT | NT | NT |
| QW-281 | 58.7 ± 11.5 | NT | NT | NT |
| QW-282 | 12.7 ± 4.7 | NT | NT | NT |
| QW-283 | −6.6 ± 8.5 | NT | NT | NT |
| QW-316 | 87.2 ± 9.2 | NT | NT | NT |
| QW-322 | 16.0 ± 4.9 | NT | NT | NT |
| QW-325 | 76.6 ± 3.9 | NT | NT | NT |
| QW-337 | 67.2 ± 3.4 | NT | NT | NT |
| QW-265 | 61.2 ± 11.3 | NT | NT | NT |
| QW-266 | 48.8 ± 3.3 | NT | NT | NT |
| QW-267 | 14.7 ± 5.7 | NT | NT | NT |
| QW-268 | −8.0 ± 15.8 | NT | NT | NT |
| QW-270 | 65.5 ± 1.6 | NT | NT | NT |
| QW-271 | 40.2 ± 5.2 | NT | NT | NT |
| QW-272 | 5.1 ± 4.8 | NT | NT | NT |
| QW-273 | −2.4 ± 7.9 | NT | NT | NT |
| QW-284 | 71.0 ± 22.2 | NT | NT | NT |
| QW-285 | 77.9 ± 7.6 | NT | NT | NT |
| QW-286 | 11.5 ± 7.1 | NT | NT | NT |
| QW-287 | −21.0 ± 11.9 | NT | NT | NT |
| QW-301 | 3.3 ± 1.2 | NT | NT | NT |
| QW-302 | 68.9 ± 1.8 | NT | NT | NT |
| QW-303 | 88.7 ± 2.3 | NT | NT | NT |
| QW-304 | 78.3 ± 4.3 | NT | NT | NT |
| QW-305 | 88.6 ± 3.1 | NT | NT | NT |
| QW-306 | 6.2 ± 7.5 | NT | NT | NT |
| QW-259 | 27.7 ± 26.1 | NT | NT | NT |
| QW-260 | 41.1 ± 4.6 | NT | NT | NT |
| QW-261 | 23.2 ± 17.0 | NT | NT | NT |
| QW-262 | −0.6 ± 7.8 | NT | NT | NT |
| QW-274 | 27.5 ± 4.4 | NT | NT | NT |
| QW-275 | 63.1 ± 3.6 | NT | NT | NT |
| QW-276 | 5.9 ± 4.4 | NT | NT | NT |
| QW-277 | 12.4 ± 20.1 | NT | NT | NT |
| QW-290 | 20.6 ± 9.0 | NT | NT | NT |
| QW-291 | 20.0 ± 4.0 | NT | NT | NT |
| QW-292 | 7.9 ± 5.3 | NT | NT | NT |
| QW-293 | −13.1 ± 14.6 | NT | NT | NT |

Data are expressed as the mean ± s.d. of each compound, n = 3 technical replicates.

The most active analogs (QW-148, QW-152, QW-156 and QW-158) in terms of inhibitory potency, were subjected to further assessment in cells. The inactive analog QW-147 was included as a negative control and known GPX4 inhibitor RSL3 was included as a positive control. Compounds were evaluated in five ferroptosis-sensitive cell lines: HT-1080 fibrosarcoma cells; HepG2, HLF, and Huh7 hepatocellular carcinoma cells; and Sk-hep-1 hepatic adenocarcinoma cells (Table 4). All cell lines were shown to be sensitive to ferroptosis induced by RSL3 with strong rescue in the presence of fer-1. All tested chloroacetyl analogs of LOC1886 were cytotoxic, but QW-148, QW-152, QW-156 and QW-158 showed some degree of fer-1 rescue at least in certain cell contexts while QW-147 showed much less fer-1 rescue across all cell contexts (FIGS. 82C-82D, Table 4). These data reinforce the idea that installing a chloroacetyl group does not guarantee GPX4 inhibition or induction of ferroptosis and suggest that the more active analogs do have GPX4-specific effects. Consistent with it showing the best binding by MST and strongest GPX4 inhibition in vitro, QW-156 was generally the most potent in cells as well (Table 5).

TABLE 4
Cellular potency and ferroptosis selectivity
of selected analogs in generation 1.

| | IC₅₀ (uM) | | IC₅₀ (uM) | | IC₅₀ (uM) | |
|---|---|---|---|---|---|---|
| Compound | HT1080^{a} | HT1080^{b} | Huh7^{a} | Huh7^{b} | HLF^{a} | HLF^{b} |
| RSL3 | 0.044 | 4.6 | 0.6 | 34.4 | 0.25 | >40 |
| QW-148 | 37.7 | 95.9 | 15.8 | 55 | 16.6 | 28.7 |
| QW-152 | 55.5 | 167.2 | 59.6 | 110.4 | 30 | 278 |
| QW-156 | 22.9 | 33.1 | 17.8 | 55.4 | 13.4 | 34.3 |
| QW-158 | 133.8 | 135.9 | 43.4 | 137.1 | 47.2 | 187.2 |
| QW-147 | 34.8 | 37.2 | 98.7 | 112.9 | 98.7 | 112.9 |

| | IC₅₀ (uM) | | IC₅₀ (uM) | |
|---|---|---|---|---|
| Compound | SKHEP1^{a} | SKHEP1^{b} | HEPG2^{a} | HEPG2^{b} |
| RSL3 | 0.05 | >40 | 5.4 | 32.2 |
| QW-148 | 10.1 | 120.6 | >400 | >400 |
| QW-152 | 24.7 | 339.8 | 81.4 | 291.4 |
| QW-156 | 13.7 | 62.8 | 25.4 | 51.5 |
| QW-158 | 45.3 | 201.8 | 95.5 | 305.7 |
| QW-147 | 24.1 | 52.8 | 69 | 103.6 |

TABLE 5
MST results of LOC1886 and its analogs are reported as
an EC50 value representing the half maximal effective
concentration due to their covalent mechanism of action.

| MST EC₅₀ | LOC1886 EC₅₀ = 262.5 μM |
|---|---|
| QW-147 EC₅₀ = 2.4 μM | QW-155 EC₅₀ = 473 μM |
| QW-144 EC₅₀ = 836 nM | QW-156 EC₅₀ = 218 nM |
| QW-148 EC₅₀ = 39.7 μM | QW-158 EC₅₀ = 836 nM |
| QW-152 EC₅₀ = 678 nM | QW-164 EC₅₀ = 2.1 μM |

Since QW-152, QW-156 and QW-158 showed the most robust inhibition of GPX4 in vitro, we further characterized their in vitro GPX4 inhibitory activities at lower concentrations. At 100 μM, 50 μM and 20 μM, QW-156 consistently showed the greatest potency (Table 3). Encouragingly, these characterizations showed a dose-dependent effect. Even at 50 μM, QW-156 showed complete GPX4 inhibition in the in vitro assay while QW-152 and QW-158 showed approximately 60% inhibition, suggesting the bromine substitution on the indole ring contributes to the potency of QW-156 (FIG. 82E, Table 3). Besides, QW-156 induced fer-1 rescuable increases in lipid peroxidation in HT-1080 cells as assessed by flow cytometry with C11-BODIPY (FIG. 82F).

To assess the GPX4 specificity of these analogs, we also tested them in a similar in vitro assay of GPX1 activity using cell lysates. Mercaptosuccinic acid (MSA), a known inhibitor of GPX1, was included as a positive control. 108 At 200 μM, these compounds show slight inhibition of GPX1 (FIG. 82G). In particular, 200 μM QW-156 showed approximately 30% GPX1 inhibition in the assay, but since QW-156 maintains complete inhibition of GPX4 even at 50 μM, there is likely a window of selectivity in which GPX4 is inhibited but other GPXs remain unaffected.

### Indazole was a Superior Pharmacophore

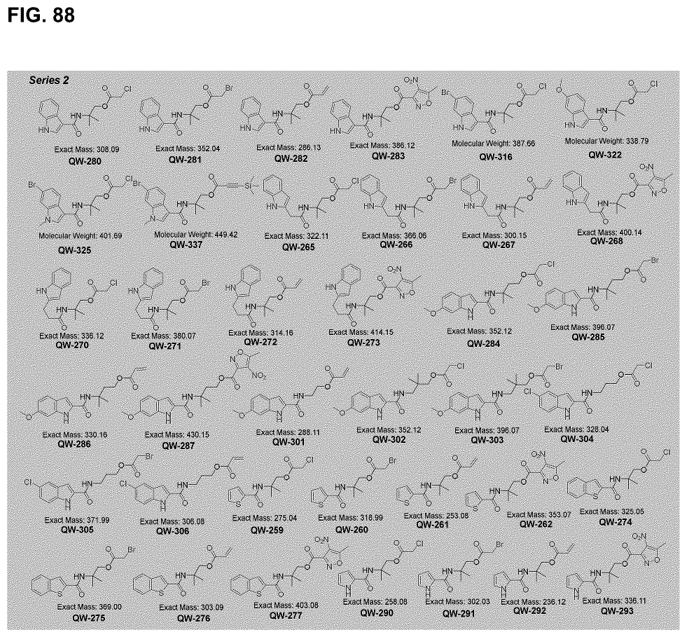

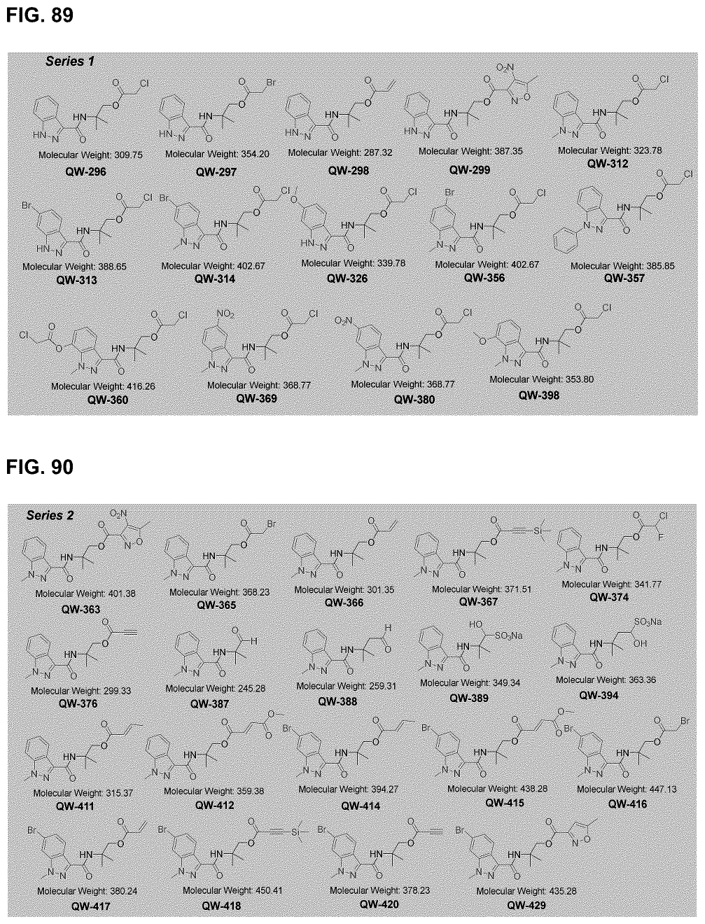

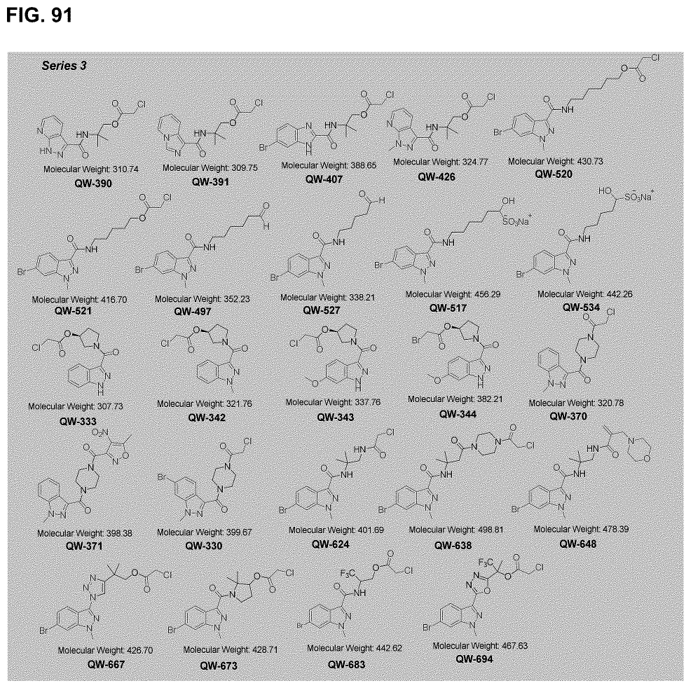

In view of the weak but apparently selective inhibition of GPX4 in certain cell lines mediated by QW-156, we decided to develop this lead further and envisaged the replacement of the indole with indazole, as indazoles are privileged structures that usually serve as indole bioisosteres. For this purpose, a set of compounds with the indazole moieties were synthesized to study the structure-activity relationships. Since QW-280 exhibited good activity with a 76% inhibition rate, the replacement of indole ring with an indazole gave indazole-3-carboxamide QW-296 and the compound displayed enhanced inhibitory activity (FIG. 89). We hypothesized that improved potency may be due to indazoles bearing two successive nitrogen atoms promotes strong donor and acceptor hydrogen bonding within the hydrophobic pockets of GPX4. Starting from QW-296, we employed two classes of modifications to this parent compound, varying the electrophiles and substitutions on the indazole, producing a initial SAR series (FIG. 89). In this series, compounds bearing N, 1-methyl (QW-312), 6-bromo (QW-313), N-methyl-6-bromo (QW-314), 5-bromo (QW-356), N-phenyl (QW-357), 7-chloroethylether (QW-360) all displayed complete inhibition against GPX4 at 200 μM concentration, comparable to QW-156 (Table 6). Evaluation at lower concentrations showed that QW-314 was the most potent among the substituted indazole analogs, exhibiting complete inhibition in the assay even at 20 μM (FIGS. 82A-82B, Table 6), which was higher than that of compound QW-156 and RSL3, suggesting indazole was a superior pharmacophore. Importantly, at that concentration, QW-314 had no inhibitory effect in the GPX1 activity assay, indicating GPX4 selectivity at that dose (FIG. 82B). We next investigated the effects of various electrophiles in the new indazole scaffolds. The assay results showed only bromomethyl ketone was tolerated from a potency standpoint (FIG. 89, Table 6). In series 2, we replaced the chloroacetyl group with a variety of electrophiles in QW-312 and QW-314, but these compounds showed vastly diminished activity except QW-365 (FIG. 90, Table 6). In the last series, we explored the effect of linker by extending the lengths and replacing all or part of the original linker with piperazine, pyrrolidine, trifluoromethyl, triazole and oxadiazole. Besides, bioisosteric replacements of the indazole ring were also evaluated. Disappointingly, these compounds exhibited a loss of GPX4 enzymatic inhibitory activity in vitro (FIG. 91, Table 6). Cellular potency and ferroptosis selectivity of selected analogs in generation 2 is shown in Table 7.

TABLE 6
GPX4 inhibition data for structural analogs of generation 2.

| | GPX4% inhibition at | | | |
|---|---|---|---|---|
| Compound | 200 (μM) | 20 (μM) | 5 (μM) | 2.5 (μM) |

Series 1

| | | | | |
|---|---|---|---|---|
| QW-296 | 83.1 ± 21.9 | NT | NT | NT |
| QW-297 | 73.1 ± 14.6 | NT | NT | NT |
| QW-298 | 7.9 ± 8.2 | NT | NT | NT |
| QW-299 | 6.3 ± 4.7 | NT | NT | NT |
| QW-312 | 95.8 ± 4.0 | 72.5 ± 6.7 | NT | NT |
| QW-313 | 97.6 ± 2.5 | 53.8 ± 12.1 | NT | NT |
| QW-314 | 93.1 ± 6.0 | 107.6 ± 5.3 | NT | NT |
| QW-326 | 84.3 ± 8.9 | NT | NT | NT |
| QW-356 | 105.7 ± 2.5 | NT | NT | NT |
| QW-357 | 104.8 ± 1.9 | NT | NT | NT |
| QW-360 | 101.9 ± 1.5 | NT | NT | NT |
| QW-369 | NT | NT | 74.6 ± 3.4 | NT |
| QW-380 | NT | NT | 62.1 ± 2.4 | NT |
| QW-398 | NT | NT | 20.3 ± 9.5 | NT |

Series 2

| | | | | |
|---|---|---|---|---|
| QW-363 | −15.4 ± 8.4 | NT | NT | NT |
| QW-365 | 94.5 ± 4.5 | NT | NT | NT |
| QW-366 | −7.3 ± 0.5 | NT | NT | NT |
| QW-367 | 7.0 ± 3.9 | NT | NT | NT |
| QW-374 | NT | 1.6 ± 3.0 | NT | NT |
| QW-376 | NT | 9.6 ± 6.0 | NT | NT |
| QW-387 | NT | 4.1 ± 4.7 | NT | NT |
| QW-388 | NT | 1.6 ± 1.9 | NT | NT |
| QW-389 | NT | 32.2 ± 2.5 | NT | NT |
| QW-394 | NT | NT | 4.5 ± 17.8 | NT |
| QW-411 | NT | NT | −6.0 ± 3.1 | NT |
| QW-412 | NT | NT | −5.7 ± 8.0 | NT |
| QW-414 | NT | NT | −9.1 ± 13.4 | NT |
| QW-415 | NT | NT | −9.4 ± 8.1 | NT |
| QW-416 | NT | NT | 25.3 ± 13.4 | NT |
| QW-417 | NT | NT | −5.2 ± 10.0 | NT |
| QW-418 | NT | NT | −5.6 ± 5.1 | NT |
| QW-420 | NT | NT | −10.9 ± 8.0 | NT |
| QW-429 | NT | NT | −2.3 ± 2.3 | NT |

Series 3

| | | | | |
|---|---|---|---|---|
| QW-390 | NT | 4.0 ± 3.9 | NT | NT |
| QW-391 | NT | 5.3 ± 8.5 | NT | NT |
| QW-407 | NT | NT | −14.3 ± 5.2 | NT |
| QW-426 | NT | NT | −11.3 ± 4.7 | NT |
| QW-520 | NT | NT | −9.8 ± 6.7 | NT |
| QW-521 | NT | NT | −19.3 ± 9.9 | NT |
| QW-497 | NT | NT | −15.1 ± 8.4 | NT |
| QW-527 | NT | NT | −24.5 ± 0.5 | NT |
| QW-517 | NT | NT | −10.7 ± 7.8 | NT |
| QW-534 | NT | NT | −36.6 ± 4.8 | NT |
| QW-333 | 17.4 ± 4.9 | NT | NT | NT |
| QW-342 | 69.4 ± 0.7 | NT | NT | NT |
| QW-343 | 40.1 ± 8.5 | NT | NT | NT |
| QW-344 | 46.2 ± 1.6 | NT | NT | NT |
| QW-370 | NT | 0.5 ± 5.4 | NT | NT |
| QW-371 | NT | 2.9 ± 2.2 | NT | NT |
| QW-330 | 33.2 ± 6.8 | NT | NT | NT |
| QW-624 | NT | NT | NT | −9.6 ± 4.4 |
| QW-638 | NT | NT | NT | 39.0 ± 26.2 |
| QW-648 | NT | NT | NT | 15.8 ± 24.7 |
| QW-667 | NT | NT | NT | 46.2 ± 31.0 |
| QW-673 | NT | NT | NT | 41.9 ± 63.4 |
| QW-683 | NT | NT | NT | −37.4 ± 5.2 |
| QW-694 | NT | NT | NT | 10.6 ± 5.8 |

TABLE 7
Cellular potency and ferroptosis selectivity
of selected analogs in generation 2.

| | IC₅₀ (uM) | | IC₅₀ (uM) | | IC₅₀ (uM) | |
|---|---|---|---|---|---|---|
| Compound | HT1080^{a} | HT1080^{b} | Huh7^{a} | Huh7^{b} | HLF^{a} | HLF^{b} |
| RSL3 | 0.044 | 4.6 | 0.6 | 34.4 | 0.25 | >40 |
| QW-303 | 4.0 | 6.1 | 6.3 | 16.8 | 2.5 | 5.7 |
| QW-305 | 1.9 | 5.0 | 3.8 | 26.1 | 0.7 | 4.3 |
| QW-312 | 5.4 | 76.3 | 4.2 | 122.5 | 2.5 | 58.1 |
| QW-313 | 20.4 | 58.8 | 17.0 | 58.2 | — | — |
| QW-314 | 1.9 | 77.4 | 1.8 | 92.9 | 0.13 | 80.0 |
| QW-348 | 8.5 | 26.0 | 4.5 | 32.4 | 4.1 | 27.8 |
| QW-356 | 3.1 | 58.3 | 2.5 | 111.8 | 1.1 | 60.9 |
| QW-357 | 9.1 | 40.1 | 3.5 | 58.7 | 2.9 | 36.4 |
| QW-360 | 12.7 | 46.7 | 9.1 | 174.7 | 6.7 | 27.1 |
| QW-365 | 0.7 | 12.2 | 0.8 | 16.8 | 0.5 | 8.8 |

| | IC₅₀ (uM) | | IC₅₀ (uM) | |
|---|---|---|---|---|
| Compound | SKHEP1^{a} | SKHEP1^{b} | HEPG2^{a} | HEPG2^{b} |
| RSL3 | 0.05 | >40 | 5.4 | 32.2 |
| QW-303 | 5.1 | 17.1 | 4.7 | 12.7 |
| QW-305 | 2.3 | 10.9 | 1.9 | 7.2 |
| QW-312 | 28.4 | 202.3 | 26.2 | 180.0 |
| QW-313 | 47.0 | 63.7 | 12.8 | 63.1 |
| QW-314 | 10.8 | 105.6 | 11.3 | 221.5 |
| QW-348 | 3.9 | 32.7 | 7.5 | 27.3 |
| QW-356 | 0.8 | 81.0 | 6.7 | 73.5 |
| QW-357 | 3.8 | 50.0 | 9.2 | 55.7 |
| QW-360 | 3.5 | 44.4 | 24.8 | 40.3 |
| QW-365 | 0.3 | 12.1 | 2.9 | 18.6 |

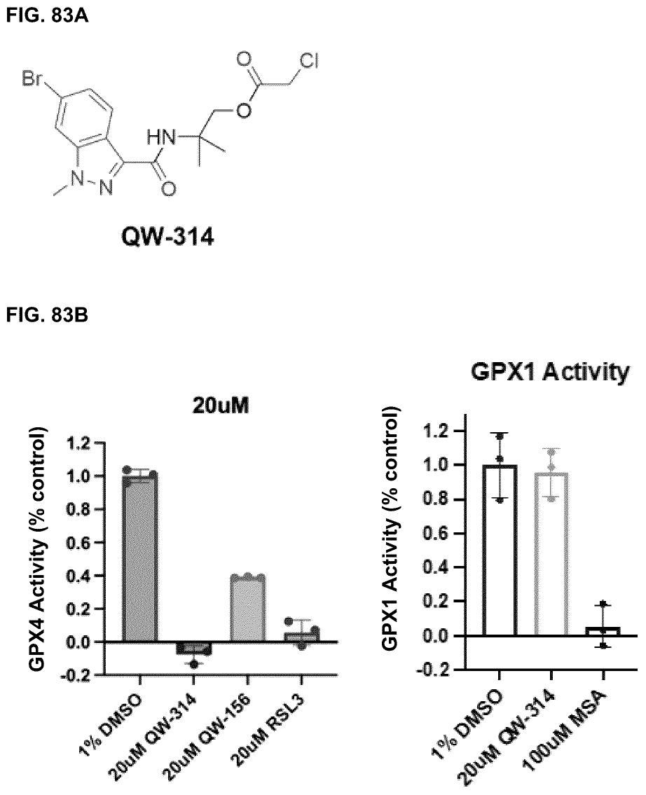

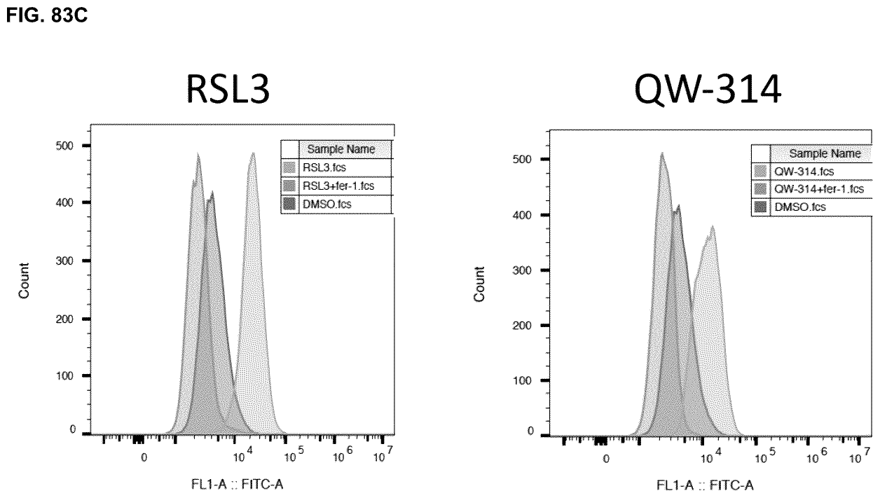

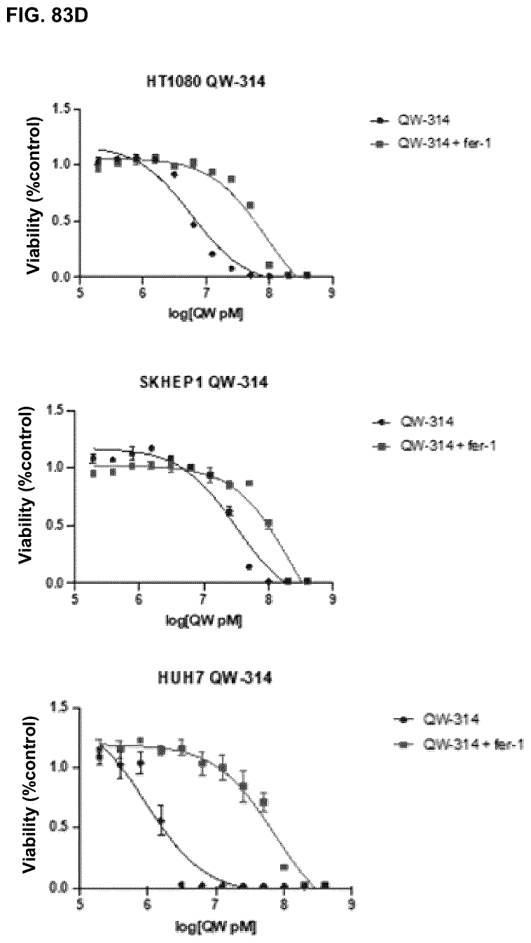

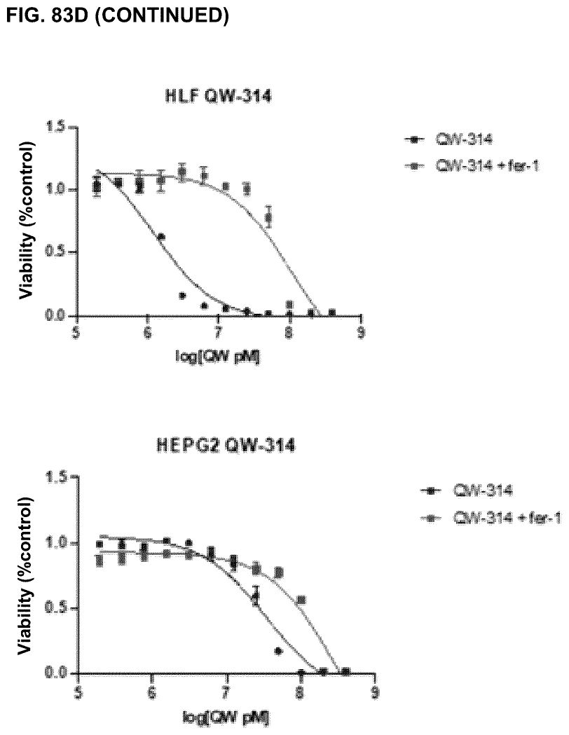

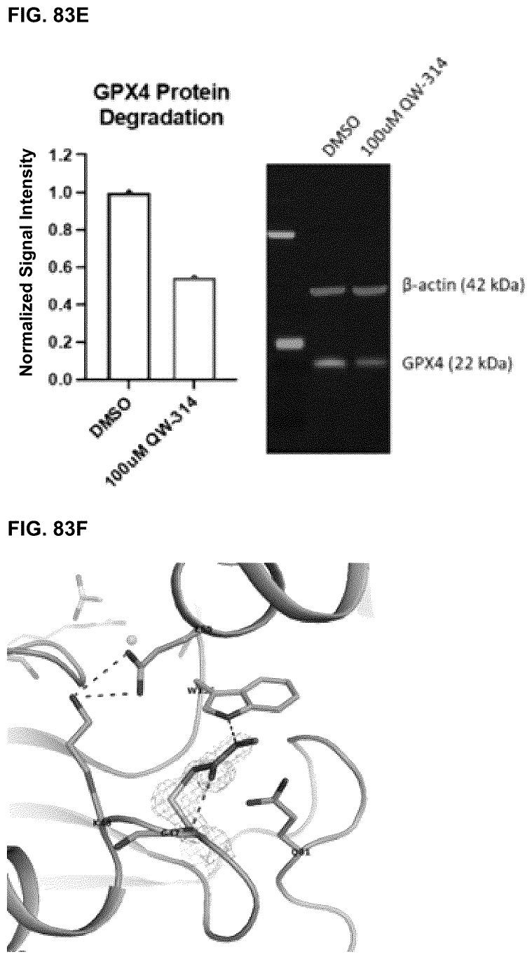

Next, QW-312, QW-313, and QW-314 were examined in the cellular context. QW-305, a compound with reduced potency compared to the substituted indazole analogs but that still showed relatively good activity in this series of SAR analyses, was included for comparison. All of the compounds induced fer-1 rescuable increases in lipid peroxidation in HT-1080 cells as assessed by flow cytometry with C11-BODIPY (FIG. 82C). The cellular potency and ferroptosis specificity were assessed in the same panel of cell lines as used previously. While QW-305 and QW-313 exhibited relatively little fer-1 rescue, implicating nonspecific cytotoxic effects (Table 6). QW-312 and QW-314 showed improved fer-1 rescue compared to the previous lead QW-156, indicating enhanced ferroptosis specificity (FIG. 83D, Table 6). Both QW-312 and QW-314 feature a 1-methylindazole group, suggesting it may play a specific role in ferroptosis induction. Consistent with the in vitro GPX4 activity data, the bromine substitution on the 1-methylindazole of QW-314 seems to confer slightly improved cellular potency over the unsubstituted analog QW-312. Additionally, by Western blot, we determined that QW-314 also induces GPX4 protein degradation in HT-1080 cells (FIG. 83E). Excitingly, we obtained co-crystal structure of the GPX4^{U46C} in complex with QW-314 (FIG. 83F). The co-crystal showed that QW-314 covalently binds to the mutant cysteine in the U46 residue, thus may target the wildtype active site.

### Extending the Scaffold Via Suzuki Reaction LED to Generation 3

The success and popularity of Suzuki-coupling reaction comes from the fact that during the drug discovery phase, this methodology is reliable and reproducible. We are also attracted to take advantage of this reaction to derivatize the lead compound to arrive at new scaffold rapidly. As QW-314 containing a bromo emerged from last generation, which is an ideal substrate for Suzuki coupling enabling the rapid expansion of structure-activity relationships (SAR). Hence, compound QW-314 was subsequently linked with a wide array of commercially available boronic acids and boronic pinacol esters by the Suzuki reaction to produce more than 100 analogues that classified in 4 series.

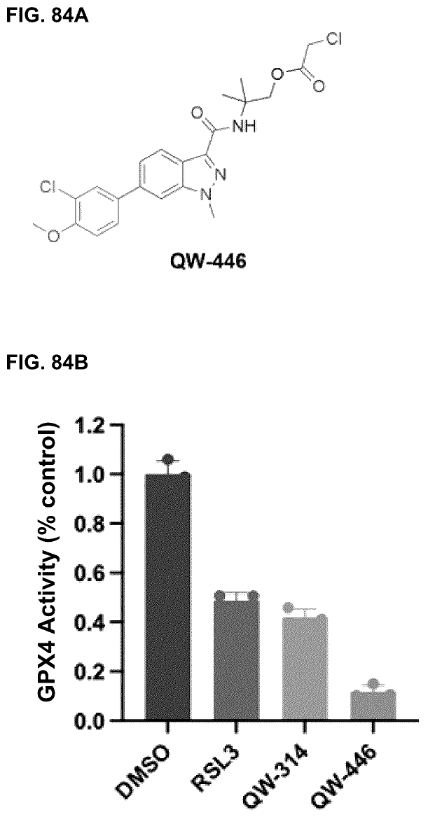

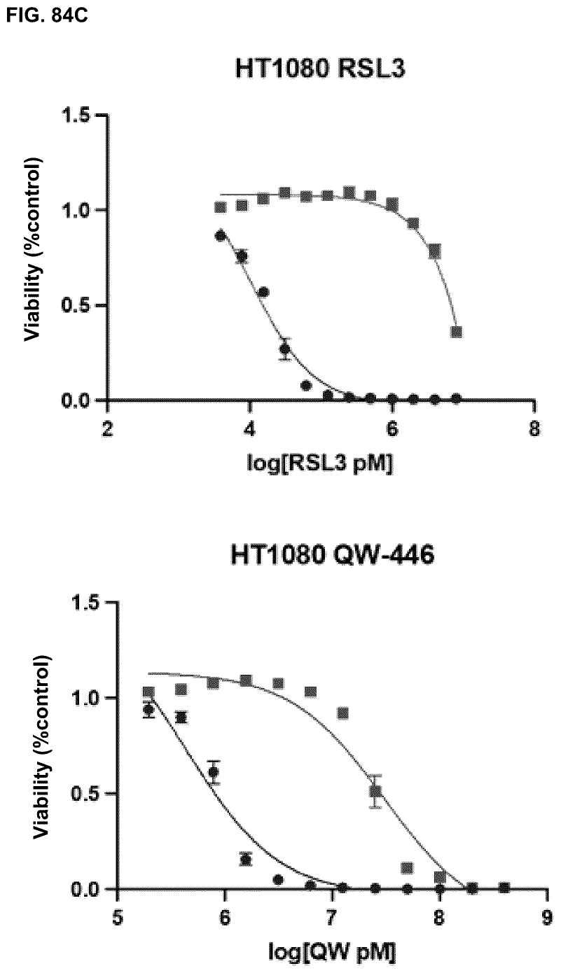

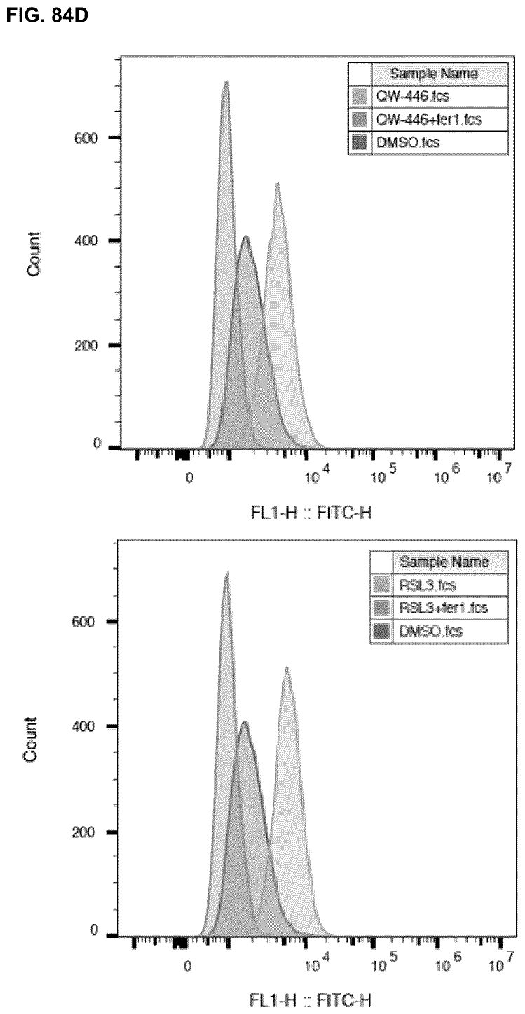

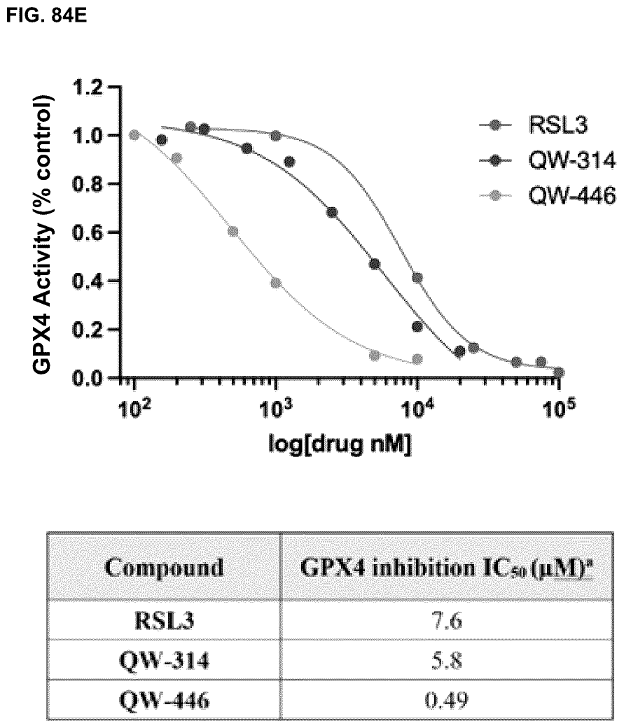

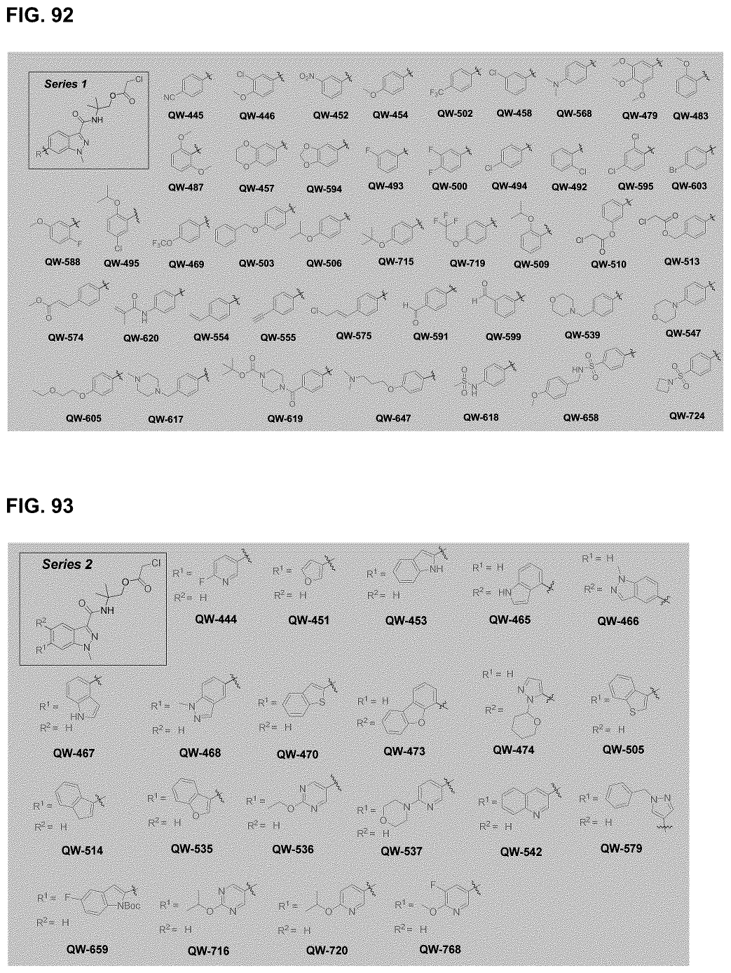

In the first series, we replaced the bromine in compound QW-314 with several different substituted benzenes at the 6-position of indazole ring (FIG. 92, Table 8). In the first small collection of 7 analogs, compounds bearing 3-nitrobenzene and 4-methylbenzene respectively, were inactive during GPX4 inhibition. Compounds QW-445 and QW-502 bearing 4-cyanobenzene and 4-trifluorobenzene on the benzene ring, respectively, were inactive during GPX4 inhibition. 3-nitrophenyl compound QW-452 and 4-Dimethylaminophenyl compound QW-568 showed higher activity, with a 50% inhibition rate. The 3-chloro-substituted analog QW-458 and 4-methoxy-substituted compound QW-452 displayed further enhanced inhibitory activity against GPX4, which were greater than that of compound QW-314. It is uplifting that the 3-chloro-4-methoxy disubstituted compound QW-446 exhibited excellent activity with a 90% inhibition rate, suggesting the synergistic effect of the two substituents (FIGS. 84A-84B). Since QW-454 and QW-458 displayed encouraging inhibitory effect, we decided to explore their analogs by enriching or replacing the substituents with original or similar substituted groups. Relocating methoxy group to the ortho-position (QW-483) displayed decreased activity against GPX4, a phenyl dimethoxy substituted (QW-487) at the ortho-positions resulted in the completely loss of the inhibitory effect. This suggests that ortho-substituents appear to be unfavorable for inhibitory effects against GPX4. Therefore, we retained the 4-methoxy and introduced additional methoxy at the C3-C5 positions of the benzene ring. 3,4,5-trimethoxyphenyl (QW-479) and 1,4-benzodioxan (QW-479) analogs exhibited a moderate inhibitory effect, but 1,3-benzodioxole analog (QW-594) showed higher activity, with a 75% inhibition rate. Furthermore, we replaced the 4-methoxy group of QW-454 with trifluoromethoxy, benzyloxy, isopropoxy, tert-butoxy and 2,2,2-trifluoroethoxy to. Among them, compound QW-715, harboring a tert-butoxy phenyl ring, completely inhibited GPX4 at the 2.5 uM. However, compound QW-719 showed no inhibition on GPX4. The other three analogs were also tested and moderate GPX4 inhibition rates were observed. Relocating the 4-isopropoxy group of QW-506 to ortho-position results in predictly dramatically decreased inhibitory effect (QW-509). Introduction of m-chloro in the benzene ring of QW-509 did not change the activity (QW-495). Subsequently, we investigated the effect of halo-substituents on the benzene ring on GPX4 inhibition. The inhibitory potencies were not significantly altered by replacing Chloro with Fluoro at 3-position (QW-493) or moving Chloro from C3 to C4 (QW-494). Compounds bearing 2-chlorobenzene (QW-492) and 4-bromobenzene (QW-603) both exhibited significantly decreased inhibitory activities than QW-458 against GPX4. The introduction of an additional halogen in the benzene ring gave compounds QW-500 and QW-595, which showed lower activities. The 2-fluoro-5-methoxyphenyl compound QW-588 was also synthesized, and a moderate GPX4 inhibition rate was observed. Then the importance of the nucleophilic substitution pattern for GPX4 activity was investigated. The presence of an additional warhead in the skeleton does not improve on the activity (QW-510, QW-513, QW-574 & QW-620), and introduction of the nucleophiles in the benzene ring like alkenes, alkyne and aldehydes (QW-554, QW-555, QW-575, QW-591 QW-599) did not change GPX4 potency significantly either. Sulfonamide, an effective bioisostere of the carboxylic group, could form a network of hydrogen bonds and improve the metabolic stability. Therefore, 3 sulfonamide-containing compounds were synthesized and the testing result indicated the potency of compounds with the sulfonamide substitution at the para-position was poor (QW-618 & QW-724). It is noteworthy that the sulfonamide substituent with a large steric hindrance (QW-658), such as N-(4-methoxybenzyl) sulfonamide, exhibited a higher inhibitory effect against GPX4. Lastly, the introduction of water-solubilizing groups, like morpholine (QW-539, QW-547), piperazine (QW-617, QW-619), ethoxyethoxy (QW-605), 3-(dimethylamino) propoxy (QW-647), exhibited moderate to good inhibitory activities, but all lower than that of QW-446.

In Series 2, the consequence of different heterocyclic substituents in the 6-position of indazole ring in QW-314 on GPX4 inhibitory activity was investigated (FIG. 93, Table 8). Although indole and N-methyl indazole are the key pharmacophores in the prior two generations, the inactivity of analogues QW-453, QW-465˜QW-468 suggested that double of these heterocycles in the skeleton is not beneficial for inhibitory potency. The analogs incorporated heterocycles in the 5-position of indazole were inactive, even though their parent compound QW-356 showed higher inhibitory potency than QW-314, suggesting the extension to this position should be avoided. Analogues comprised furan, indene and benzofuran rings displayed weak to moderate inhibitory activities, and strikingly, introduction of a benzothiophene and quinoline ring results in comparable potencies to QW-446. Furthermore, introduction of a 6-isopropoxypyridine (QW-720) improved the potency, and replacing the pyridine with pyrimidine (QW-716) further increased inhibitory potency, which both exhibited substantially higher GPX4 inhibitory activities than QW-446. However, some replacements of the isopropoxy in the heterocycles converted the high-potency analogues into very low-potency analogues (QW-444, QW-536, QW-537 & QW-768).

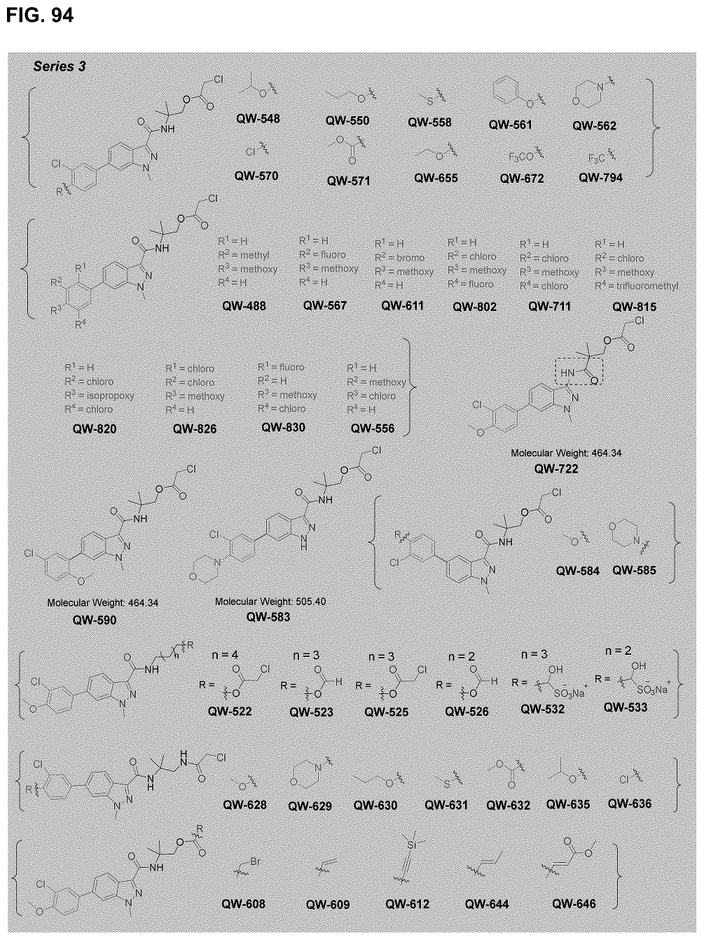

In view of the significant inhibition of GPX4 mediated by compound QW-446, we decided to develop this lead further and to explore its SAR. This was done by the synthesis and testing of a number of structural analogues of QW-446 in the series 3 (FIG. 94, Table 8). Initially, the 4-methoxy of the phenyl ring in QW-446 was replaced with various groups or moieties and these analogues displayed roughly comparable or slightly higher inhibitory potencies to QW-446. Notably, QW-562 and QW-655, the 4-morpholine and 4-ethoxy substituents in the benzene ring of QW-446, both exhibited slightly higher potencies than their parent compound. Subsequently, we replaced the 3-chloro in the benzene with methyl, fluoro and bromo, respectively, resulting in slightly to significantly decreased potencies of the compound. This suggests that 3-chloro substitution pattern in the benzene ring was found to be an important contributor to inhibitory activity against GPX4. Furthermore, introduction of 5-fluoro and 5-trifluoromethyl in the phenyl ring of QW-446 did not impact inhibitory potency substantially, but an additional 5-chloro substituent further increase the inhibitory activity (QW-711). Interchanging the positions of the 3-chloro and 4-methoxy substituents in QW-446 reduced the inhibitory activity of the compound (QW-556). Similar, changing the sequence of amide in QW-446 results in complete loss of inhibitory activity. Predictably, relocating the 4-methoxy to 6-position resulted in the loss of the inhibitory effect. Similarly, moving 3-chloro-4-methoxy phenyl ring in QW-446 or 3-chloro-4-morpholine phenyl ring in QW-562 from 6-position to 5-position in the indazole ring resulted in inactive analogues. It is noteworthy that removal of the methyl at the N1 nitrogen in the active analogue QW-562 induced slight loss of inhibitory activity (QW-583), indicating the methyl substitutent in the position contributes to the inhibitory activity. Considering that the ester bond might have undesirable pharmacokinetic properties, we thus hypothesized that a bioisosteric ester-to-amide substitution could lead to improvement in metabolic stability. Thus, we replaced the ester with amide in several compounds with satisfactory GPX4 inhibitory activities. However, all the newly achieved analogs displayed dramatically decreased inhibitory potencies, which means that the ester-to-amide strategy had a negative effect on the maintenance of inhibitory effect. This may because the modification from ester to amide group could affect the hydrogen bond profile. In order to further improve the potency and metabolic stability, we also performed a bioisosteric amide-to-triazole substitution, since the triazole group is considered a privileged scaffold and often present in the structure of marketed drugs. Although the triazole-containing analogs generally significantly decreased the inhibitory potencies, compound QW-671 can retain some potency, which was suitable for the subsequent metabolic stability study. Next, 1-isopropyl indazole and 5 structurally diverse warheads were used to replace 1-methyl indazole and chloroacetyl for improve the potency and drug likeness. Still, all of the synthesized target compounds displayed very limited activities.

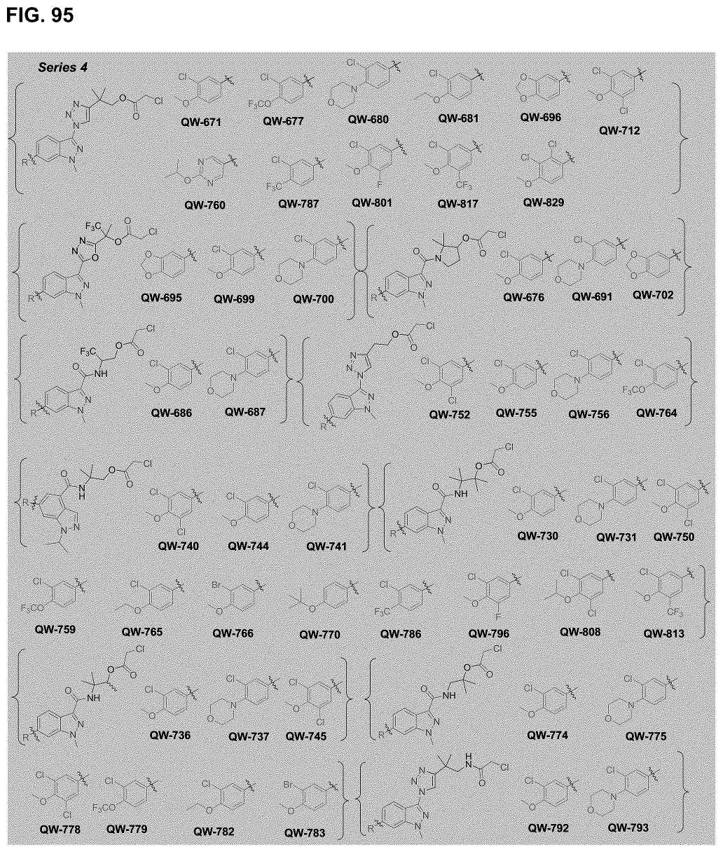

Series 4 consisted of miscellaneous analogues in which the classic linker present in all previous analogues in generations 1-3 was modified or substituted for other linkers (FIG. 95, Table 8). Analogues, in which the original linker were replaced with the long side chain-eliminating alkyl linker, shorter oxadiazole-containing linker, constrained pyrrolidine-containing linker and flexible trifluoro-branched linker, displayed negligible inhibitory potencies, which seems indicative of a limit to the modification allowed in the linker. Since ester was found to be detrimental to inhibitory activity, we still decided to modify the classic linker for improved potency and metabolic stability. Thus dimethyl and mono methyl side chains were attached to the carbon adjacent to ester in the original linker. Surprisingly, displacements of the 2-methylpropyl linker with an 2,2,3,3-tetramethylbutanyl or 2,2,3-trimethylbutanyl linkers in QW-446 and QW-562, yielded analogues QW-730, QW-731, QW-736 and QW-737 that displayed comparable and improved inhibitory potencies to their parent compounds. Relocating the dimethyl group from the carbon adjacent to amide to the carbon adjacent to ester results in loss of inhibitory activities against GPX4, this underlines the importance of the dimethyl substitution at that position.

The compounds emerged in this generation with great inhibitory activities were subjected examined in the cellular context. Given the sensibility of ferroptosis in HT1080 cells, the cytotoxicity of all the prepared compounds was evaluated in vitro against only human fibrosarcoma HT1080. QW-446 had the best cellular potency with a IC₅₀ value of 0.44 μM and the ferroptosis selectivity with a value of 69, while QW-454, QW-655, QW-815 also showed sub-molar range cellular activities and decreased ferroptosis selectivity (FIG. 84C, Table 8). It is worth mentioning analogs QW-671 and QW-730 of QW-446 which designed for improving metabolic stability retained the cellular potency somewhat. More analogs exhibited significantly reduced cytotoxicity despite of their good GPX4 inhibitory potencies (Table 8). QW-446 induced fer-1 rescuable increases in lipid peroxidation in HT-1080 cells as assessed by flow cytometry with C11-BODIPY (FIG. 84D). Moreover, the IC₅₀ of QW-446 in the in vitro GPX4 activity assay was determined to be 0.49 μM, which is much better to that of QW-314 at 5.8 μM and RSL3 at 7.6 μM (FIG. 84E). Molecular docking of QW-446 is shown in FIG. 84F.

TABLE 8
Potency and ferroptosis selectivity of 3^{rd} generation analogs.

| | | | IC₅₀ (uM) | | |
|---|---|---|---|---|---|
| | GPX4% inhibition at | | | HT1080 + | |
| Compound | 5 μM | 2.5 μM | HT1080 | Fer-1 | Selectivity |

Series 1

| | | | | | |
|---|---|---|---|---|---|
| QW-445 | 21.2 ± 5.7 | NT | NT | NT | NT |
| QW-446 | 88.1 ± 2.7 | 79.1 ± 4.4 | 0.44 | 30.3 | 69 |
| QW-452 | 55.0 ± 5.8 | NT | NT | NT | NT |
| QW-454 | 86.8 ± 2.6 | NT | 0.94 | 11.5 | 12.2 |
| QW-502 | −10.2 ± 6.5 | NT | NT | NT | NT |
| QW-458 | 80.5 ± 3.0 | NT | 2.0 | 24.0 | 12 |
| QW-568 | 67.5 ± 3.9 | NT | NT | NT | NT |
| QW-479 | 59.6 ± 6.8 | NT | NT | NT | NT |
| QW-483 | 35.8 ± 7.4 | NT | NT | NT | NT |
| QW-487 | 3.3 ± 3.0 | NT | NT | NT | NT |
| QW-457 | 54.4 ± 7.2 | NT | NT | NT | NT |
| QW-594 | NT | 71.5 ± 3.0 | 2.7 | 8.8 | 3.3 |
| QW-493 | 77.1 ± 10.4 | NT | NT | NT | NT |
| QW-500 | 57.5 ± 5.3 | NT | NT | NT | NT |
| QW-494 | 76.8 ± 2.3 | NT | NT | NT | NT |
| QW-492 | 45.7 ± 4.9 | NT | NT | NT | NT |
| QW-595 | NT | 36.4 ± 4.9 | NT | NT | NT |
| QW-603 | NT | 45.5 ± 4.4 | NT | NT | NT |
| QW-588 | 59.5 ± 5.7 | NT | NT | NT | NT |
| QW-495 | 22.9 ± 7.5 | NT | NT | NT | NT |
| QW-469 | 64.3 ± 0.7 | NT | NT | NT | NT |
| QW-503 | 65.3 ± 3.2 | NT | NT | NT | NT |
| QW-506 | 75.4 ± 3.9 | NT | NT | NT | NT |
| QW-715 | NT | 95.3 ± 7.6 | 2.6 | 24.1 | 9.3 |
| QW-719 | NT | 2.4 ± 17.3 | NT | NT | NT |
| QW-509 | 28.3 ± 3.0 | NT | NT | NT | NT |
| QW-510 | 67.2 ± 1.9 | NT | NT | NT | NT |
| QW-513 | 50.1 ± 2.4 | NT | NT | NT | NT |
| QW-574 | 72.8 ± 7.7 | NT | NT | NT | NT |
| QW-620 | NT | 63.8 ± 7.8 | NT | NT | NT |
| QW-554 | 41.4 ± 4.5 | NT | NT | NT | NT |
| QW-555 | 20.2 ± 0.5 | NT | NT | NT | NT |
| QW-575 | 35.9 ± 1.3 | NT | NT | NT | NT |
| QW-591 | 69.4 ± 3.3 | NT | NT | NT | NT |
| QW-599 | NT | 51.0 ± 4.4 | NT | NT | NT |
| QW-539 | 49.6 ± 4.5 | NT | NT | NT | NT |
| QW-605 | NT | 60.9 ± 15.7 | 1.9 | 17.5 | 9.2 |
| QW-617 | NT | 80.9 ± 3.4 | NT | NT | NT |
| QW-618 | NT | 18.1 ± 5.9 | NT | NT | NT |
| QW-619 | NT | 42.6 ± 8.0 | NT | NT | NT |
| QW-658 | 20.9 ± 14.0 | 26.3 ± 63.2 | 3.5 | 14.6 | 4.2 |
| QW-647 | 20.9 ± 14.0 | 46.9 ± 35.9 | NT | NT | NT |
| QW-724 | NT | 23.8 ± 3.7 | NT | NT | NT |
| QW-547 | 72.0 ± 0.4 | NT | NT | NT | NT |

Series 2

| | | | | | |
|---|---|---|---|---|---|
| QW-444 | 34.1 ± 0.3 | NT | NT | NT | NT |
| QW-451 | 37.7 ± 4.1 | NT | NT | NT | NT |
| QW-453 | 67.0 ± 4.7 | NT | NT | NT | NT |
| QW-465 | 7.7 ± 6.8 | NT | NT | NT | NT |
| QW-466 | 8.1 ± 2.2 | NT | NT | NT | NT |
| QW-467 | −10.3 ± 4.3 | NT | NT | NT | NT |
| QW-468 | 39.0 ± 3.9 | NT | NT | NT | NT |
| QW-470 | 75.0 ± 5.6 | NT | 1.7 | 17.3 | 10.2 |
| QW-473 | 4.7 ± 5.4 | NT | NT | NT | NT |
| QW-474 | 1.0 ± 8.7 | NT | NT | NT | NT |
| QW-505 | 76.1 ± 4.0 | NT | NT | NT | NT |
| QW-514 | 44.8 ± 6.6 | NT | NT | NT | NT |
| QW-535 | 62.2 ± 5.1 | NT | NT | NT | NT |
| QW-536 | 46.6 ± 2.3 | NT | NT | NT | NT |
| QW-537 | 46.1 ± 2.7 | NT | NT | NT | NT |
| QW-542 | 75.6 ± 4.5 | NT | NT | NT | NT |
| QW-579 | 66.3 ± 3.4 | NT | NT | NT | NT |
| QW-659 | 8.5 ± 13.4 | −1.2 ± 15.2 | NT | NT | NT |
| QW-716 | NT | 86.4 ± 7.3 | 1.6 | 63.0 | 39.4 |
| QW-720 | NT | 76.9 ± 4.2 | NT | NT | NT |
| QW-768 | NT | 32.6 ± 7.9 | NT | NT | NT |

Series 3

| | | | | | |
|---|---|---|---|---|---|
| QW-548 | 91.0 ± 2.8 | NT | NT | NT | NT |
| QW-550 | 91.5 ± 2.2 | NT | NT | NT | NT |
| QW-558 | 91.2 ± 2.3 | NT | NT | NT | NT |
| QW-561 | 87.6 ± 2.8 | NT | NT | NT | NT |
| QW-562 | 95.8 ± 2.0 | 77.6 ± 12.0 | 1.1 | 7.2 | 6.5 |
| QW-570 | 80.7 ± 14.8 | NT | NT | NT | NT |
| QW-571 | 82.5 ± 5.2 | NT | NT | NT | NT |
| QW-655 | NT | 106.9 ± 5.6 | 0.7 | 15.6 | 22.3 |
| QW-672 | NT | 68.0 ± 45.2 | 2.4 | 18.2 | 7.6 |
| QW-794 | NT | 78.5 ± 10.5 | NT | NT | NT |
| QW-488 | 87.3 ± 3.3 | NT | NT | NT | NT |
| QW-567 | 54.7 ± 3.4 | NT | NT | NT | NT |
| QW-611 | NT | 81.5 ± 1.9 | NT | NT | NT |
| QW-802 | NT | 80.1 ± 4.7 | NT | NT | NT |
| QW-711 | NT | 98.4 ± 17.6 | 1.4 | 31.7 | 22.6 |
| QW-815 | NT | 79.3 ± 5.5 | 0.58 | 16.7 | 28.8 |
| QW-820 | NT | 71.4 ± 6.9 | NT | NT | NT |
| QW-826 | NT | 66.2 ± 5.4 | NT | NT | NT |
| QW-830 | NT | 65.5 ± 5.9 | NT | NT | NT |
| QW-556 | 80.2 ± 1.8 | NT | NT | NT | NT |
| QW-722 | NT | −2.2 ± 5.0 | NT | NT | NT |
| QW-590 | 53.5 ± 6.3 | NT | NT | NT | NT |
| QW-583 | 70.9 ± 6.7 | NT | NT | NT | NT |
| QW-584 | 9.5 ± 5.1 | NT | NT | NT | NT |
| QW-585 | 23.3 ± 5.2 | NT | NT | NT | NT |
| QW-522 | −13.6 ± 5.7 | NT | NT | NT | NT |
| QW-523 | −28.7 ± 6.5 | NT | NT | NT | NT |
| QW-525 | −22.3 ± 7.8 | NT | NT | NT | NT |
| QW-526 | −33.4 ± 12.0 | NT | NT | NT | NT |
| QW-532 | −21.2 ± 13.3 | NT | NT | NT | NT |
| QW-533 | −34.7 ± 9.8 | NT | NT | NT | NT |
| QW-628 | NT | 33.6 ± 6.5 | NT | NT | NT |
| QW-629 | NT | 14.9 ± 16.7 | NT | NT | NT |
| QW-630 | NT | 30.9 ± 33.4 | NT | NT | NT |
| QW-631 | NT | 14.3 ± 16.5 | NT | NT | NT |
| QW-632 | NT | −4.9 ± 21.8 | NT | NT | NT |
| QW-635 | NT | 14.0 ± 15.4 | NT | NT | NT |
| QW-636 | 76.4 ± 32.7 | 50.2 ± 22.0 | NT | NT | NT |
| QW-608 | NT | 72.2 ± 5.9 | NT | NT | NT |
| QW-609 | NT | 6.7 ± 11.9 | NT | NT | NT |
| QW-612 | NT | −5.7 ± 11.7 | NT | NT | NT |
| QW-644 | NT | 31.4 ± 18.5 | NT | NT | NT |
| QW-646 | 30.6 ± 12.2 | 52.0 ± 27.0 | NT | NT | NT |

Series 4

| | | | | | |
|---|---|---|---|---|---|
| QW-671 | NT | 70.3 ± 20.4 | 0.73 | 16.0 | 21.9 |
| QW-677 | NT | 33.6 ± 6.3 | 1.65 | 14.0 | 8.5 |
| QW-680 | NT | 57.3 ± 2.8 | 3.0 | 12.1 | 4.0 |
| QW-681 | NT | 31.0 ± 4.9 | NT | NT | NT |
| QW-696 | NT | 22.3 ± 37.1 | 2.9 | 8.8 | 3.0 |
| QW-712 | NT | 68.5 ± 9.4 | 1.9 | 28.4 | 14.9 |
| QW-760 | NT | 14.6 ± 12.4 | NT | NT | NT |
| QW-787 | NT | 35.1 ± 29.1 | NT | NT | NT |
| QW-801 | NT | 58.1 ± 9.2 | 8.4 | 14.3 | 1.7 |
| QW-817 | NT | 75.3 ± 14.5 | NT | NT | NT |
| QW-829 | NT | 68.7 ± 3.6 | NT | NT | NT |
| QW-695 | NT | −49.5 ± 28.5 | 9.4 | 14.4 | 1.5 |
| QW-699 | NT | 2.4 ± 17.3 | NT | NT | NT |
| QW-700 | NT | −2.2 ± 5.0 | NT | NT | NT |
| QW-676 | NT | 22.9 ± 12.1 | NT | NT | NT |
| QW-691 | NT | 30.5 ± 7.4 | NT | NT | NT |
| QW-702 | NT | 23.8 ± 3.7 | NT | NT | NT |
| QW-686 | NT | 35.8 ± 14.3 | NT | NT | NT |
| QW-687 | NT | 7.9 ± 2.3 | NT | NT | NT |
| QW-752 | NT | −6.6 ± 9.7 | NT | NT | NT |
| QW-755 | NT | 43.2 ± 14.9 | 9.5 | 23.9 | 2.5 |
| QW-756 | NT | 41.6 ± 9.0 | NT | NT | NT |
| QW-764 | NT | 25.4 ± 12.0 | NT | NT | NT |
| QW-740 | NT | −153.3 ± 26.2 | NT | NT | NT |
| QW-741 | NT | −173.4 ± 12.2 | NT | NT | NT |
| QW-744 | NT | −132.7 ± 1.4 | 4.2 | 6.7 | 1.6 |
| QW-730 | NT | 98.4 ± 17.6 | 0.59 | 5.7 | 9.7 |
| QW-731 | NT | 68.5 ± 9.4 | 1.2 | 2.8 | 2.3 |
| QW-750 | NT | 20.4 ± 3.6 | 1.6 | 5.4 | 3.4 |
| QW-759 | NT | 18.4 ± 8.3 | NT | NT | NT |
| QW-765 | NT | 12.0 ± 10.7 | NT | NT | NT |
| QW-766 | NT | −31.1 ± 29.6 | 1.2 | 2.5 | 2.1 |
| QW-770 | NT | −36.4 ± 19.3 | 1.0 | 2.2 | 2.2 |
| QW-786 | NT | 31.7 ± 19.5 | 2.4 | 5.8 | 2.6 |
| QW-796 | NT | 4.6 ± 14.4 | 2.4 | 2.6 | 1.1 |
| QW-808 | NT | 36.3 ± 16.1 | NT | NT | NT |
| QW-813 | NT | −17.0 ± 28.7 | 2.1 | 3.0 | 1.4 |
| QW-736 | NT | 95.3 ± 7.6 | 3.7 | 7.3 | 2.0 |
| QW-737 | NT | 86.4 ± 7.3 | 1.0 | 1.7 | 1.7 |
| QW-745 | NT | 55.4 ± 8.1 | 1.6 | 1.8 | 1.1 |
| QW-774 | NT | −25.3 ± 6.9 | 3.7 | 8.8 | 2.4 |
| QW-775 | NT | 36.2 ± 4.1 | NT | NT | NT |
| QW-778 | NT | 30.4 ± 10.0 | NT | NT | NT |
| QW-779 | NT | 39.1 ± 17.7 | NT | NT | NT |
| QW-782 | NT | −1.0 ± 32.8 | NT | NT | NT |
| QW-783 | NT | −172.4 ± 10.3 | NT | NT | NT |
| QW-792 | NT | −19.6 ± 18.2 | 1.1 | 5.8 | 5.3 |
| QW-793 | NT | −53.4 ± 17.0 | NT | NT | NT |

### Pharmacokinetic Profile of Lead Compounds

With lead compounds in hand, we conducted preliminary assessments of ADMET properties and the results was summarized in Table 9. First, the aqueous solubility of lead compounds were determined. Verapamil and tamoxifen were included as representative controls of good and poor aqueous solubility, respectively, and three known GPX4 inhibitors, 26a, ML162 and RSL3, were included for comparison. Table 9 shows that a few compounds exhibited improved aqueous solubility compared to the known GPX4 inhibitors. Among them, QW-446 performed the best with an aqueous solubility up to 1000 μM.

TABLE 9
Pharmacokinetic Profile of lead compounds.

| | | Plasma Stability | | Microsome Stability | |
|---|---|---|---|---|---|
| | Solubility | t_{1/2} (min) | | t_{1/2} (min) | |
| Compound | (μM) | human | mouse | human | mouse |
| RSL3 | 26 | >1000 | 5.2 | 14 | 2.4 |
| ML162 | 3.8 | — | — | 0.9 | 1.5 |
| 26a | 7.2 | 195 | 80.5 | 1.5 | 1.5 |
| Verapamil | 87 | — | — | — | — |
| Tamoxifen | 5 | — | — | — | — |
| Testosterone | — | — | — | 9.5 | 5.3 |
| Ibrutinib | — | 797 | >1000 | | |
| LOC1886 | — | 10.8 | <2 | — | — |
| QW-152 | 88 | — | — | 1.5 | 0.9 |
| QW-156 | 23 | — | — | >1000 | >1000 |
| QW-158 | 83 | — | — | 4.2 | >1000 |
| QW-171 | 56 | — | — | 187 | 283 |
| QW-312 | 98 | — | — | 2.9 | 1.7 |
| QW-313 | 47 | — | — | 9.7 | 12.1 |
| QW-314 | 61 | 2.3 | 40.7 | 6.0 | 5.3 |
| QW-316 | 71 | — | — | 24 | 6 |
| QW-446 | >1000 | 7.5 | 24.0 | 1.6 | 1.2 |
| QW-454 | 2.9 | — | — | 1.8 | 1.7 |
| QW-458 | 6.1 | — | — | 1.9 | 1.9 |
| QW-488 | 5.4 | — | — | 1.8 | 1.1 |
| QW-505 | <1.3 | 10.2 | 6.3 | 1.7 | 1.8 |
| QW-506 | 1.7 | — | — | 2.9 | 1.7 |
| QW-548 | 7.8 | 49.3 | 6.7 | 42 | 30 |
| QW-550 | 7.8 | 73.2 | 12.5 | 49 | 25 |
| QW-561 | 15.6 | 116 | 23.9 | 12 | 24 |
| QW-562 | 7.8 | 7.4 | 5.3 | — | — |
| QW-570 | 15.6 | 56.7 | 16.9 | 22 | 15 |
| QW-594 | 1.0 | 6.6 | 17.0 | 250 | 137 |
| QW-624 | — | 142 | 685 | 8.8 | 1.2 |
| QW-628 | — | 243 | 311 | 15 | 19 |
| QW-655 | — | 16.8 | 2.6 | — | — |
| QW-658 | — | 12.5 | 6.2 | | |
| QW-671 | — | 44.5 | 6.1 | 33 | 31 |
| QW-672 | — | 47.9 | 4.7 | 17 | 19 |
| QW-680 | — | 67.4 | 3.2 | 28 | 26 |
| QW-686 | — | 16.6 | 23.8 | | |
| QW-730 | — | 318 | 385 | 6.6 | 10.7 |
| QW-737 | — | 275 | 6.6 | 8.6 | 7.4 |
| QW-745 | — | 245 | 25.4 | 19.5 | 13.0 |
| QW-750 | — | 290 | >1000 | 10.2 | 13.5 |

Next, the metabolic stability of these compounds was evaluated in human and mouse plasma or liver microsomes (Table 9). While QW-156 showed the excellent microsome stability in both human and mouse liver microsomes, QW-158 exhibited specificity to mouse microsome. Compounds QW-152 and QW-171 revealed the substitution pattern is an important structural feature for microsome stability. Notably, QW-316, the regioisomer of QW-156, showed the loss of microsome stabilities. Indazole analogs QW-312, QW-313 and QW-314 had similar microsome stability in comparison to QW-316, indicating their molecular geometries were unfavourable for microsome stability. QW-313 showed better microsome stability than QW-312 and QW-314, suggesting methylation of the indazole ring presents a metabolic liability. Attaching a substituted aromatic ring to indazole in QW-314 did not provide some improvement in microsome stability. Surprisingly, QW-446 was also less stable in plasma and microsomes relative to QW-314 despite of the excellent aqueous solubility. QW-446 analogs having larger substituted groups or electron withdrawing group, such as isopropoxy (QW-548), isopropoxy (QW-550), phenoxy (QW-561) and chloro (QW-570), in place of the methoxy showed improved microsome stability and human plasma properties, as well as the trifluoromethoxy analog (QW-672). However, morpholine (QW-562) and ethoxy (QW-655) analogs have similar potencies in plasma stability relative to QW-446, indicating methoxy, morpholine and ethoxy are potential metabolic weak spots. Properties of compounds QW-624 and QW-628 serve to demonstrate methyl ester group is a contributor to the poor plasma stability. Bioisosteric analogs QW-671 and QW-680 having triazole in place of the amide had improved stability in the microsomes and human plasma, but not as significant as the ester-to-amide strategy. Lastly, an examination of linkers identified that the addition of dimethyl group to the carbon adjacent to the ester group (QW-730 & QW-750) can considerately stabilize the compounds in plasma, whereas monomethyl introduction to the same site showed improved human plasma stability properties. This suggested that conformationally constrained chloroacetyl ester is less susceptible to being metabolized in plasma. Particularly, only methylenedioxy analog QW-594 showed much better stability in microsomes in comparison to QW-446.

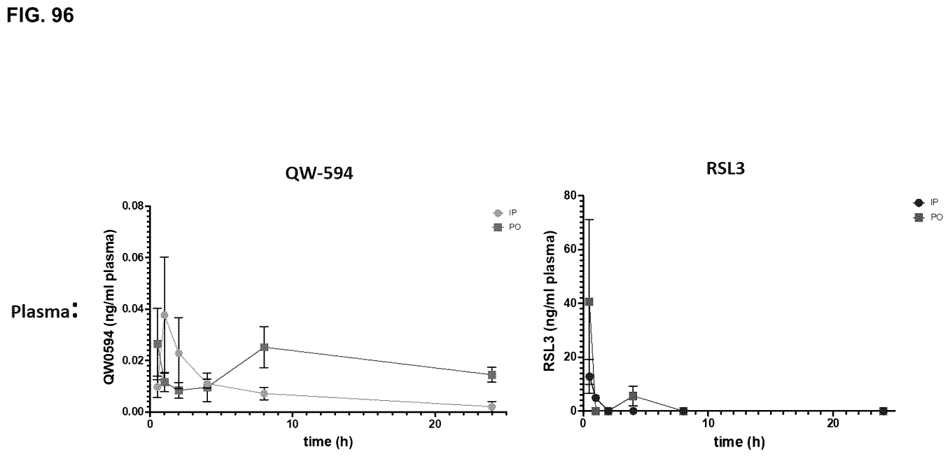

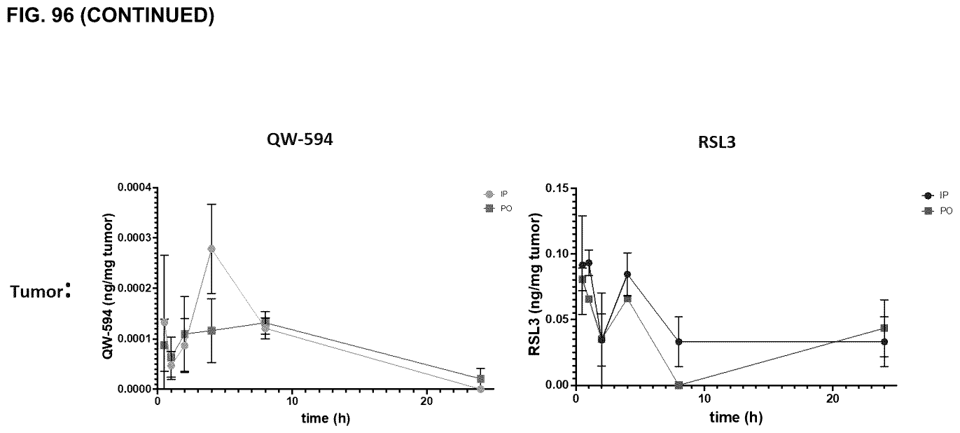

Subsequently, a pharmacokinetic study of QW-594 was performed in vivo (FIG. 96). For the pharmacokinetic study, we measured the molar concentration of QW-594 and RSL3 in plasma and tumor of NCG mouse after treatments with 20 mg/kg QW-594 or RSL3 via intraperitoneal (IP) or oral (PO) dose. Unfortunately, both of IP and PO administrations did not show significant accumulation of QW-594 in plasma. Since QW-594 has good microsomal stability, with an unbound clearance rate of 5.1 μL/h/mg, the extremely high plasma clearance indicated extra-hepatic clearance mechanisms. Besides, QW-594 has poor stability in mouse plasma, with a half-life of 17 min, which undoubtedly contributes to the high clearance in vivo.

Longitudinal Extension LED to Discovery of Analogs with High Potencies in Generation 4

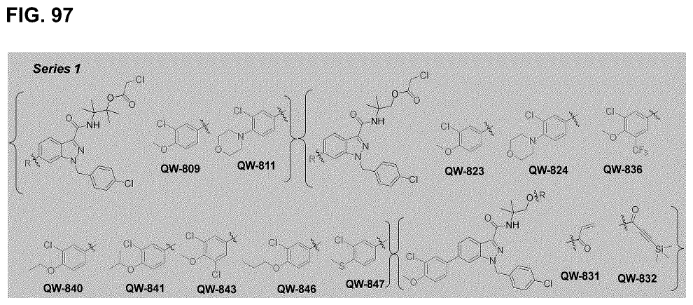

In our previous study, we found that the methyl substituted at the 1-position of the indazole scaffold is beneficial for inhibitory activity improvement, this observation served as inspiration for us to further longitudinally explore structural extensions of the compounds with the aim of augmenting their potencies. Besides, the methyl group is a likely contributor to the poor microsome stability, which could potentially be blocked in derivatives. Therefore, we initially replaced the methyl group in lead compounds with 4-chlorobenzyl group at the N, 1-position of indazole in the lead compounds (FIG. 97). The purpose of this modification was to introduce a bulkier side chain to increase the longitudinal steric hindrance of the compound, thereby verifying our hypothesis that longitudinal extension might enhance the inhibitory potency. In series 1, the 1-(4-chlorobenzyl)-indazole derivative QW-823 and QW-824 showed improved GPX4 inhibitory activity compared with that of the parent compounds QW-446 and QW-562, respectively. However, this strategy did not help the potency improvement when the substrates bearing 2,2,3,3-tetramethylbutanyl linkers (QW-809 & QW-811), and chloromethyl ketone still behave as the best warhead in the new leads (QW-831 & QW-832) (Table 10). Several analogs were incorporated with 4-chlorobenzyl group to replace methyl group in the indazole core and these compounds displayed comparable or improved GPX4 activities except QW-836, with a 50% inhibition rates at 2.5 μM. Subsequently, the cytotoxicity and ferroptosis selectivity of these new leads with GPX4 enzyme inhibitory activities were evaluated. Among them, compounds QW-823, QW-824 and QW-841 showed outstanding cytotoxicity with sub-molar scale, accompanied by good ferroptosis selectivity. From above, this vertical extension strategy was indeed beneficial to GPX4 potency.

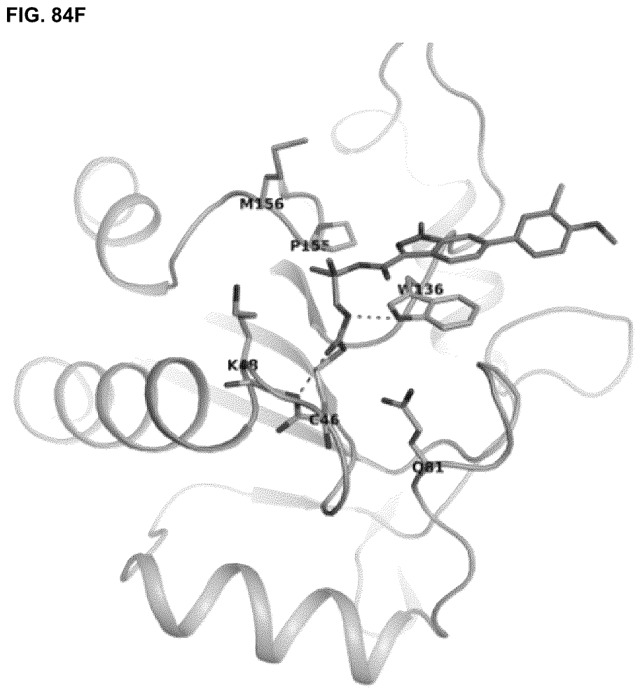

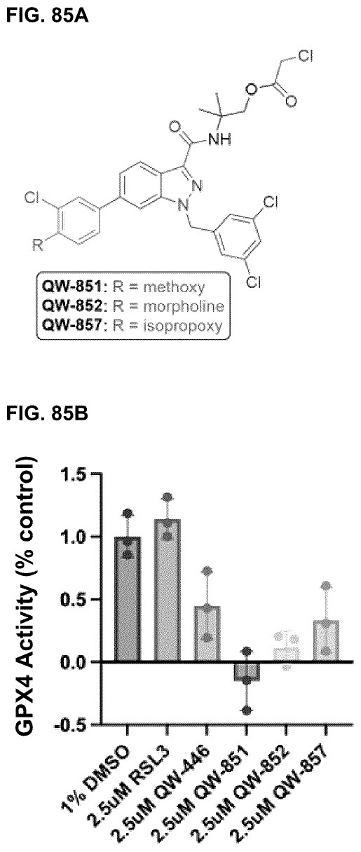

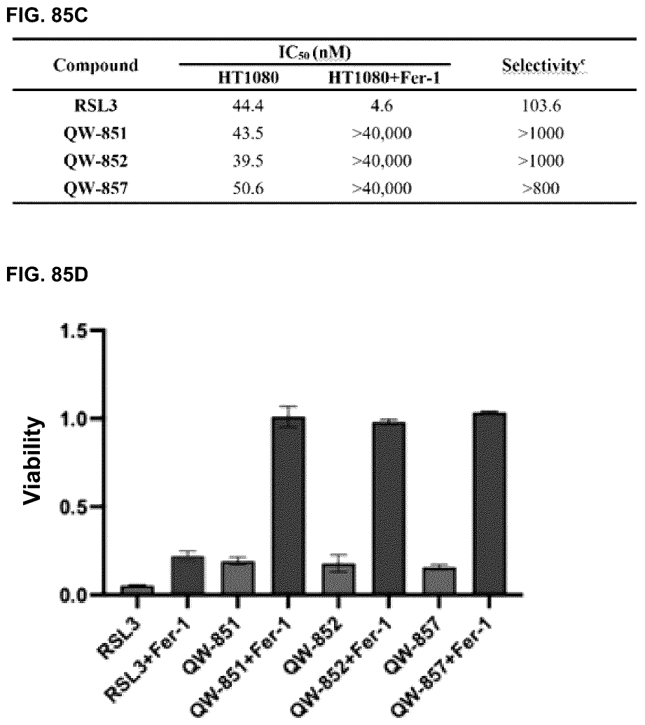

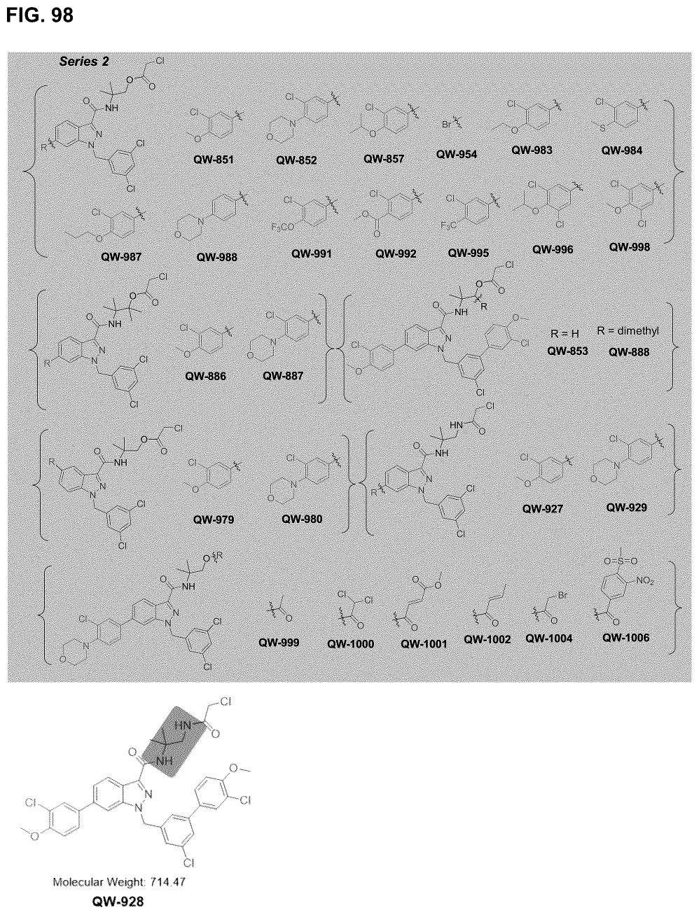

We next investigated the effects of various substituents in the benzyl ring attached to the indazole on GPX4 inhibitory activity (FIG. 98, Table 10). In our second series, we removed the 4-Cl and introduced chloro substituents at the C3 and C5 positions of the benzyl ring. This modification of the benzyl ring (QW-851, QW-852 & QW-857) (FIG. 85A) not only results in excellent GPX4 inhibitory activity (FIG. 85B), but also markedly enhanced cytotoxicity against HT1080 cell lines with IC₅₀ of 43.5 nM, 39.5 nM and 50.6 nM, respectively, which are better or comparable to than RSL3 (FIG. 85C). And the ferroptosis selectivity was dramatically improved as well. Compound QW-446 revealed that the benzyl group on the indazole is important to the activity of QW-851, since replacing benzyl group with methyl resulted in a 10-fold increase in potency in the cell viability assay (FIG. 85D). When a chloro in the benzyl ring of the compound QW-851 was replaced, which exhibited significantly decreased the inhibitory activity and cytotoxicity. Intriguingly, with moderate GPX4 inhibitory activity, compound QW-857 showed significant cytotoxicity and ferroptosis selectivity, suggesting 3,5-dichloro substituents in the benzyl ring played a crucial role in the cytotoxicity of these compounds. Subsequently, we transplanted this important feature to more lead compounds and synthesized a small collection of analogs with 3,5-dichlorobenzyl ring. In general, these compounds showed dramatically reduced cytotoxicity, although some of them showed satisfactory cytotoxicity with sub-molar range. Besides, the exploration of more stable linker and drug-like warheads was discouraging, as well as ester-to-amide strategy. By the way, relocating the phenyl substituents to the 5-position in the indazole ring of QW-851 and QW-852 dramatically decreased the cytotoxicity and ferroptosis selectivity. Noteworthy, we prepared QW-999, a derivative of QW-852 by replacing the chlorine atom with a hydrogen atom in the chloromethylketone, was inactive in cellular assay. This suggested QW-852 inhibits GPX4 in a covalent manner.

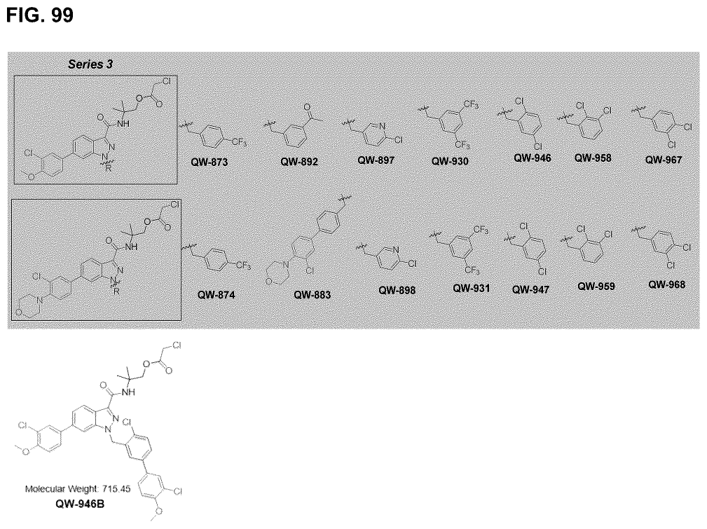

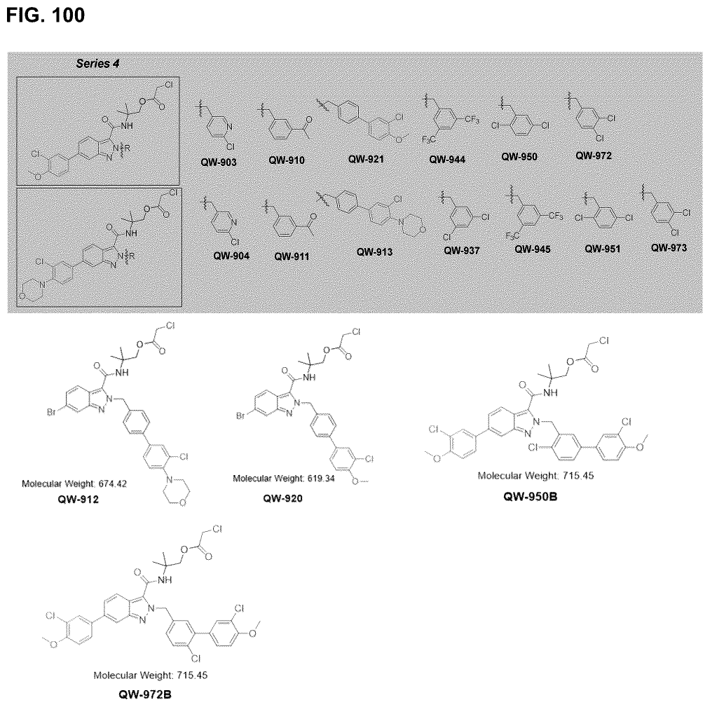

Furthermore, we introduced more different substituents in the benzyl moiety and measured the potency of these compounds on HT1080 cells (FIG. 99, Table 10). Introduction of trifluoromethyl, 3-chloro-4-morpholine, acetyl or pyridine in the to the benzyl group led to a dramatical decrease in the cellular activity and ferroptosis selectivity. Besides, we investigated the effect of the chlorine positions in QW-851 and QW-852. Moving the chloro randomly was not tolerant, and only QW-947 bearing a 2,5-benzyl group showed good cytotoxicity and ferroptosis selectivity, which is still not comparable to the parent compound QW-852. Lastly, the regioisomeric analogs with various different substitution patterns in the benzyl group generally showed diminished cellular potency and ferroptosis selectivity (FIG. 100, Table 10). For instance, the regioisomer of QW-852 showed 25-fold reduced activity (QW-937 IC₅₀ of 1 μM). Compounds QW-904, QW-950, QW-951 retained cytotoxicity for HT-1080 cells (sub-molar scale), whereas showed the big loss of ferroptosis selectivity with a value of 3.8, 6.9, 5.4, respectively.

TABLE 10
Potency and ferroptosis selectivity of 4^{th} generation analogs.

| | GPX4% | IC₅₀ (μM) | | |
|---|---|---|---|---|
| | inhibition at | | HT1080 + | |
| Compound | 2.5 μM | HT1080 | Fer-1 | Selectivity |

Series 1

| QW-809 | 34.6 ± 29.6 | 4.2 | | 6.5 | 1.5 |
|---|---|---|---|---|---|
| RSL3 | | 44.4 | nM | 4.6 | 103.6 |

| | | | | |
|---|---|---|---|---|
| QW-811 | 47.4 ± 8.9 | 0.28 | 12.7 | 45.4 |
| QW-823 | 95.0 ± 13.1 | 0.33 | 11.4 | 34.5 |
| QW-824 | 90.5 ± 7.4 | 0.18 | >40 uM | >220 |
| QW-831 | 32.6 ± 61.0 | 12.0 | 13.0 | 1.1 |
| QW-832 | 58.9 ± 9.0 | 1.7 | 63.6 | 37.4 |
| QW-836 | 45.7 ± 17.5 | — | — | — |
| QW-840 | 80.1 ± 11.8 | — | — | — |
| QW-841 | 91.3 ± 2.0 | 0.74 | 28.5 | 38.5 |
| QW-843 | 76.8 ± 11.4 | — | — | — |
| QW-846 | 91.5 ± 3.9 | — | — | — |
| QW-847 | 88.5 ± 16.1 | 10.2 | 98.4 | 9.6 |

Series 2

| QW-851 | 114.9 ± 23.5 | 43.5 | nM | >40 uM | >920 |
|---|---|---|---|---|---|
| QW-852 | 88.2 ± 13.3 | 39.5 | nM | >40 uM | >1000 |
| QW-853 | 45.5 ± 34.3 | 0.37 | | 21.0 | 56.8 |
| QW-857 | 66.7 ± 26.2 | 50.6 | nM | >40 uM | >790 |
| QW-886 | NT | 12.4 | | 55.3 | 4.5 |
| QW-887 | NT | 232.7 | nM | >40 uM | >174 |

| | | | | |
|---|---|---|---|---|
| QW-888 | 78.7 ± 5.0 | 24.5 | 49.6 | 2.0 |
| QW-954 | NT | 5.9 | 11.8 | 2.0 |
| QW-983 | NT | 2.2 | 19.2 | 8.7 |
| QW-984 | NT | 0.75 | >40 | >53 |
| QW-987 | NT | 0.72 | >40 | >55 |
| QW-988 | NT | 2.9 | 19.5 | 6.7 |
| QW-991 | NT | 0.66 | 21.1 | 32.0 |
| QW-992 | NT | 3.1 | 23.3 | 7.5 |
| QW-995 | NT | 42.3 | 47.1 | 1.1 |
| QW-996 | NT | 0.72 | 12.8 | 17.8 |
| QW-998 | NT | 0.35 | >40 | >114.3 |
| QW-979 | NT | 6.9 | 12.5 | 1.8 |
| QW-980 | NT | 5.5 | 8.4 | 1.5 |
| QW-927 | NT | 1.6 | 8.2 | 5.1 |
| QW-928 | NT | — | — | NT |
| QW-929 | NT | 1.6 | 4.6 | 2.9 |
| QW-999 | NT | >40 | >40 | — |
| QW-1000 | NT | >40 | >40 | — |
| QW-1001 | NT | >40 | >40 | — |
| QW-1002 | NT | >40 | >40 | — |
| QW-1004 | NT | 3.8 | 8.9 | 2.3 |
| QW-1006 | NT | >40 | >40 | — |

Series 3

| QW-873 | 45.6 ± 39.7 | 8.0 | | 26.8 | 3.4 |
|---|---|---|---|---|---|
| QW-874 | 57.5 ± 24.5 | 28.3 | | 49.0 | 1.7 |
| QW-883 | 41.1 ± 40.9 | 56.4 | nM | 20.8 | 371.4 |

| | | | | |
|---|---|---|---|---|
| QW-892 | NT | 23.7 | 37.7 | 1.6 |
| QW-897 | NT | 1.2 | 9.0 | 7.5 |
| QW-898 | NT | 1.2 | 8.1 | 6.8 |
| QW-930 | NT | 21.8 | 13.0 | 0.6 |
| QW-931 | NT | 5.2 | 34.3 | 6.6 |
| QW-946 | NT | 3.4 | 453.6 | 133.4 |
| QW-947 | NT | 0.34 | 61.7 | 181.5 |
| QW-958 | NT | 2.3 | 31.6 | 13.7 |
| QW-959 | NT | 1.2 | 610.2 | 508.5 |
| QW-967 | NT | 894.2 | 28.7 | 32.1 |
| QW-968 | NT | >40 | >40 | — |

Series 4

| | | | | |
|---|---|---|---|---|
| QW-903 | NT | — | — | NT |
| QW-904 | NT | 0.74 | 2.8 | 3.8 |
| QW-910 | NT | NT | NT | NT |
| QW-911 | NT | 1.3 | 5.0 | 3.8 |
| QW-912 | NT | 1.5 | 5.1 | 3.4 |
| QW-913 | NT | NT | NT | NT |
| QW-920 | NT | NT | NT | NT |
| QW-921 | NT | NT | NT | NT |
| QW-937 | NT | 1.0 | 2.7 | 2.7 |
| QW-944 | NT | NT | NT | NT |
| QW-945 | NT | 2.7 | 10.7 | 4.0 |
| QW-950 | NT | 0.42 | 2.9 | 6.9 |
| QW-951 | NT | 0.52 | 2.8 | 5.4 |
| QW-972 | NT | 3.6 | 5.5 | 1.5 |
| QW-973 | NT | 4.2 | 5.8 | 1.4 |

### Lead Compounds Assessment in Drug-Tolerant Persister (DTP) Cell Models

Drug-tolerant persister (DTP) cells are a small tumoral subpopulation implicated in post-chemotherapeutic cancer relapse and have been shown to be uniquely sensitive to GPX4 inhibitor-induced ferroptosis. To assess the therapeutic applicability and as another measure of GPX4 specificity in cells, a well-established non-small cell lung carcinoma DTP cell model derived from erlotinib treatment of PC9 cells was used. the most promising lead compound in each generation were selected and tested in a DTP cell model. Known GPX4 inhibitor RSL3 was included as a positive control. The DTP potency of this small selection of the compounds is summarized in Table 11.

TABLE 11
Viability of lung (PC9) cancer parental and
persister cells treated with selected leads.

| | | % Viability w/STD (all technical quadruplicate) | | | | | |
|---|---|---|---|---|---|---|---|
| Com- | Dose | | | | | Persister + | |
| pound | (μM) | PC9 | SD | Persister | SD | 2 μM Fer-1 | SD |
| RSL3 | 1.0 | 85.00 | 12.03 | 6.00 | 1.16 | NA | NA |
| 156 | 1.0 | 148.50 | 18.66 | 122.00 | 12.78 | NA | NA |
| 156 | 10.0 | 57.25 | 13.23 | 73.25 | 19.75 | NA | NA |
| 156 | 25.0 | 3.25 | 6.50 | 24.50 | 11.39 | NA | NA |
| 156 | 50.0 | 1.00 | 0.00 | 2.50 | 0.58 | NA | NA |
| 314 | 10.0 | 62.00 | 4.76 | 22.00 | 8.91 | 73.00 | 5.83 |
| 446 | 0.5 | 84.25 | 4.11 | 127.75 | 34.85 | 110.75 | 15.69 |
| 446 | 10.0 | 74.75 | 6.65 | 26.50 | 26.66 | 107.50 | 5.07 |
| 446 | 20.0 | 28.75 | 1.71 | 4.25 | 1.50 | 27.00 | 12.75 |
| 811 | 1.0 | 92.00 | 2.45 | 93.00 | 3.74 | NA | NA |
| 811 | 10.0 | 77.25 | 5.32 | 4.75 | 0.96 | NA | NA |
| 852 | 1.0 | 81.25 | 5.56 | 48.00 | 7.75 | 93.75 | 3.86 |
| 852 | 10.0 | 45.50 | 3.00 | 3.25 | 0.96 | NA | NA |

As expected, DTP cells showed increased sensitivity to RSL3-induced cell death compared to the parental PC9 cells. Intriguingly, while QW-156 showed no selectivity for DTP cells over PC9 cells at most concentrations tested, QW-314 exhibited preferential induction of significantly more cell death in the DTP cells compared to the parental cells at 10 μM drug treatments. QW-446 and QW-811 also showed selective and similar or stronger cytotoxicity to DTP cells at 10 μM tested concentration, and QW-446 can be fully rescued by Fer-1. Additionally, QW-852 exhibited greater potency and selectivity to DTP cells, and showed a hallmark of ferroptosis. These data are consistent with the improved fer-1 rescue observed in the cellular dose-response assays and suggest enhanced GPX4 inhibitor-specific ferroptosis induction in the cellular context. In summary, we successfully prepared several novel GPX4 inhibitors with good potencies and selectivities against DTP cells, thus may provide a innovative opportunity to prevent drug tolerance and resistance by killing drug-tolerant cells before they give rise to fully resistant tumors, and they could be combined with immune therapy to more effectively eliminate the DTP cells and manage cancer.

### Discussion

Given the importance of GPX4 in tumor recurrence and persister cell survival, many efforts have been made to develop specific and potent GPX4 inhibitors in the past decade. Nonetheless, these inhibitors have demonstrated difficulty in being developed as drugs. Much effort was still put into the development of the existing inhibitors such as RSL3, and ML162 via structural optimization, or converting them into new modalities like proteolysis targeting chimeras (PROTACs) and antibody-drug conjugates (ADC) for high efficiency and selectivity and the capacity of overcoming resistance. But the results of these efforts have been minimal.

In the present study, we used LOC1886 as the chemical template for five rounds of structure-activity relationship (SAR)-guided synthesis with the goal of identifying novel and potent GPX4-selective lead compounds. These diverse libraries provide valuable insight into the SAR driving the GPX4 ligand affinity, efficacy, and target selectivity. We demonstrated that QW-852, as a novel GPX4 inhibitor, effectively inhibited GPX4 with potent cytotoxicity and specificity to ferroptosis in Human HT-1080 Fibrosarcoma Cells in vitro. QW-852 is a potential candidate worthy of further development for clinical application, particularly in hepatocellular carcinoma, which lack effective therapeutic approaches.

Although QW-852 is very potent, investigation of water solubility, plasma stability and microsome stability were needed for in vivo experiments. For therapeutic development, further improvement on its ADMET properties is needed. To this end, determination of co-crystal structures of QW-852 in complex with GPX4 is critical and will provide us with the solid foundation to perform structure-based optimization toward ultimate development of a GPX4-targeted therapy.

### Example 11

### Further Investigations on Lead GPX4 Inhibitors

As described in the Examples above, two lead candidates, VP-224 (based on RSL3 scaffold) and QW-852 (based on LOC1886), were synthesized for targeting GPX4. This Example provided further results on these two compounds.

### VP-224 and QW-852 Induce Ferroptosis in Cancer Cells in Vitro

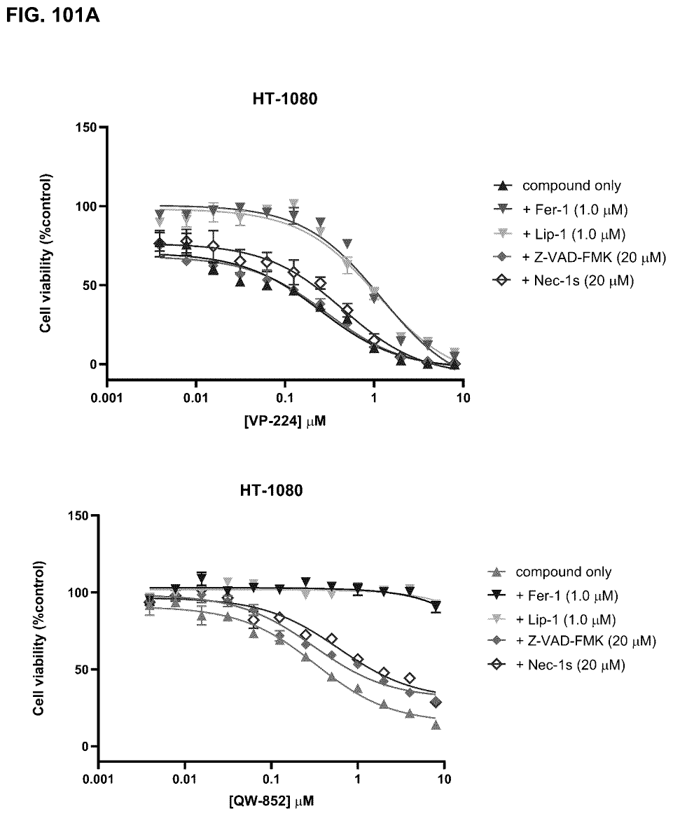

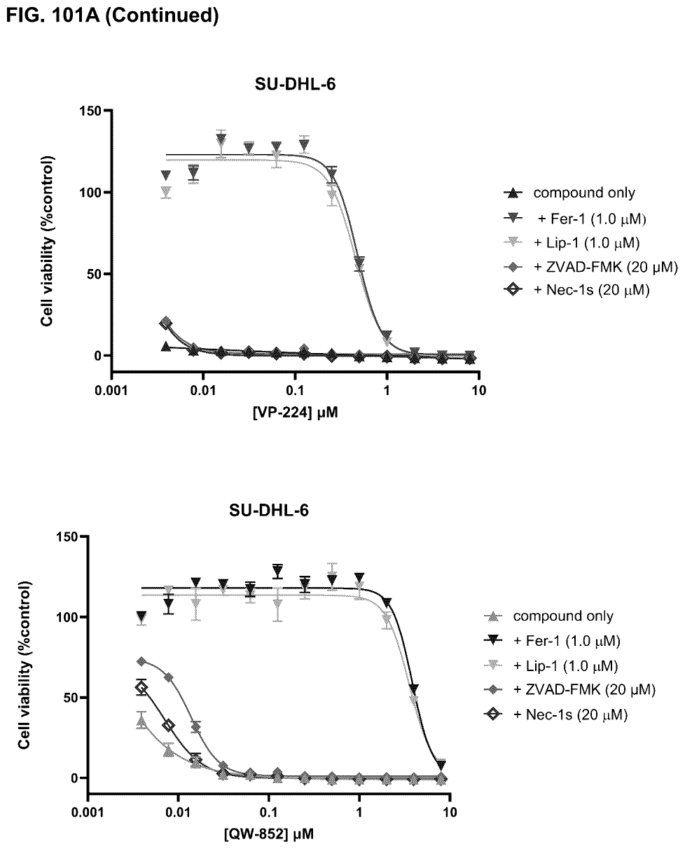

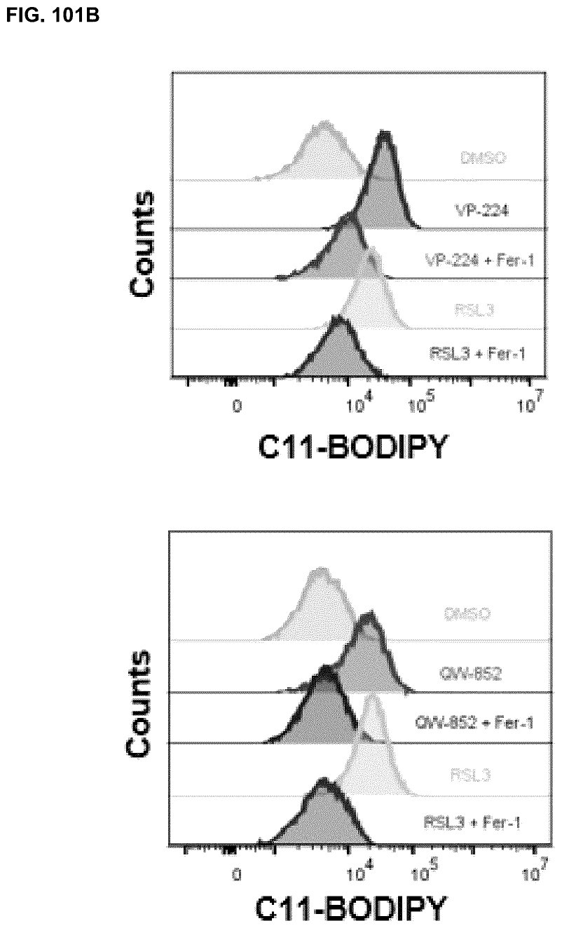

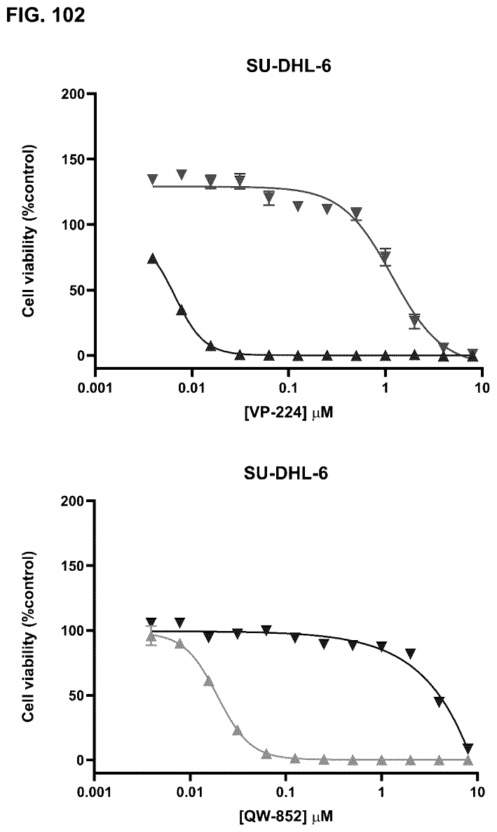

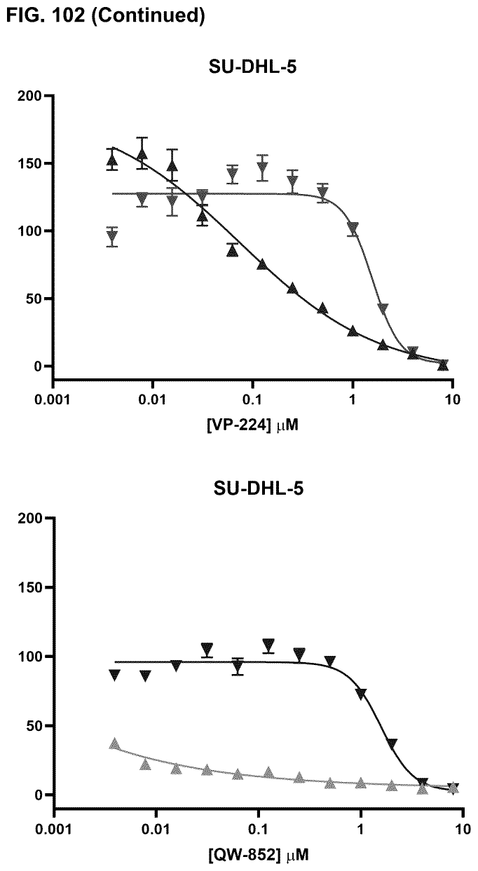

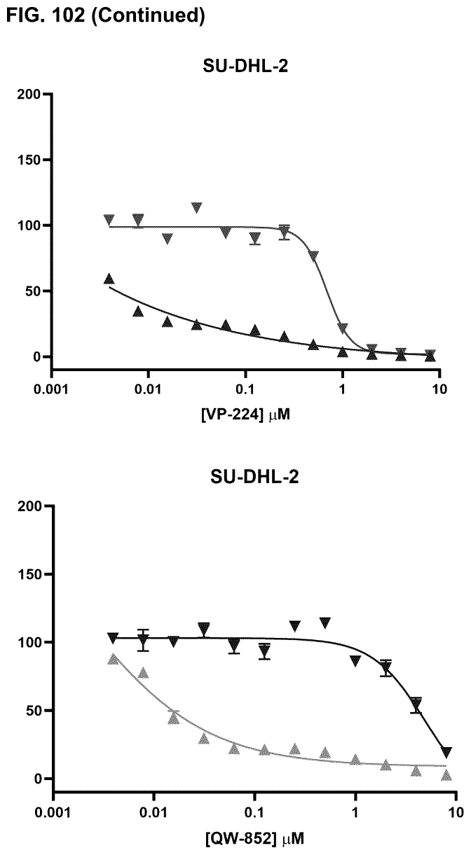

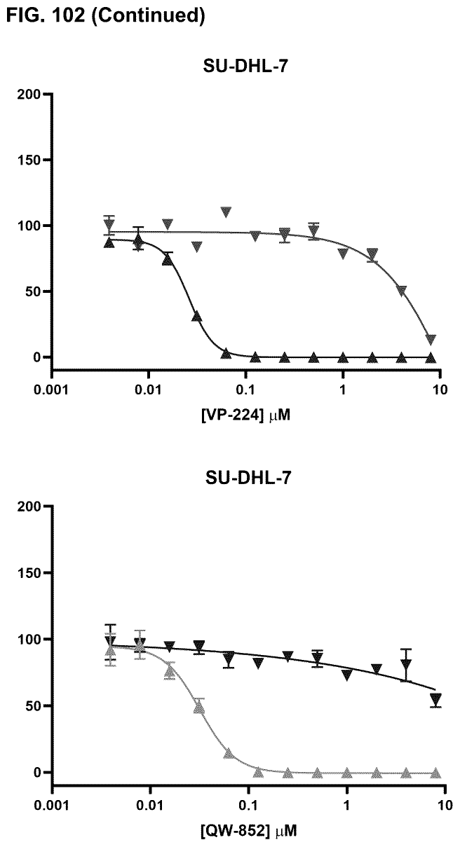

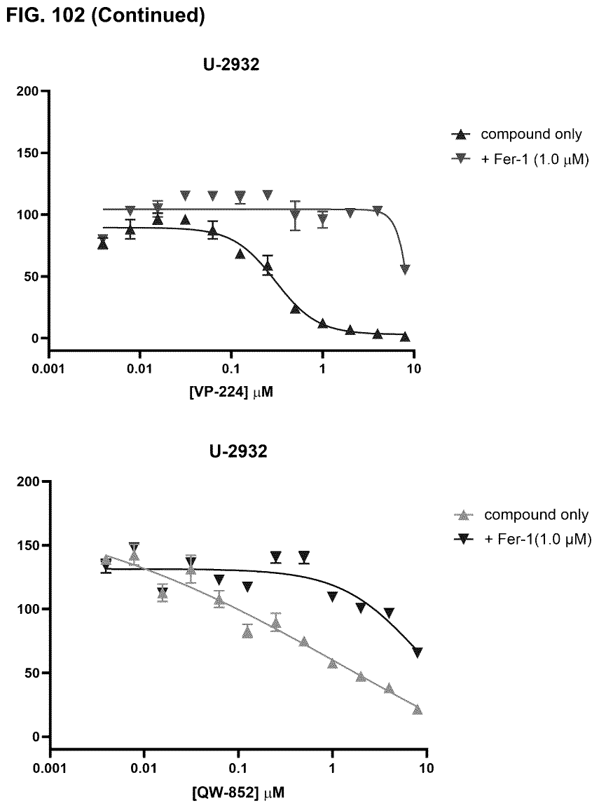

The cytotoxicity of VP-224 and QW-852 were evaluated across several diffuse large B-cell lymphomas (DLBCL) cell lines, including both “IKE-sensitive” (SU-DHL-5, SU-DHL-6, SU-DHL-2) and “IKE-resistant” (SU-DHL-7 and U-2932) cell lines as previously described (Zhang et al. 2019). Both compounds exhibited nanomolar potency (Table 12 and FIG. 102). Previously reported, ferroptosis inducer RSL3 was also included for comparison. Furthermore, treatment with ferrostatin-1 (1.0 μM), a ferroptosis inhibitor, could reverse VP-224 and QW-852 cytotoxity, confirming that the induced cell death was through ferroptosis (Table 12 and FIG. 102). To determine whether other forms of cell death contribute to the cytotoxic effects of VP-224 and QW-852, HT-1080 and SU-DHL-6 cells were co-treated with those two compounds and other cell death inhibitors, including the apoptosis inhibitor Z-VAD-FMK, the necroptosis inhibitor necrostatin-1s (Nec-1s), and the ferroptosis inhibitors ferrostatin-1 (Fer-1) and liproxstatin (Lip-1); only the ferroptosis inhibitors blocked the cytotoxic effect of VP-224 and QW-852 (FIG. 101A). It was also found that both compounds can cause an increase in lipid-ROS which can be attenuated by ferrostatin-1, as measured by using the fluorescent lipid peroxidation marker C11-BODIPY in flow cytometry (FIG. 101B).

TABLE 12
VP-224 and QW-852 activity and selectivity
in different cancer cell lines.

| | | | IC₅₀ (μM) | Selectivity^{a} |
|---|---|---|---|---|
| Compound | Cell line | IC₅₀ (μM) | with Fer-1 | (fold) |
| 1 (VP-224) | SU-DHL-6 | 0.007 | 1.18 | 169 |
| 2 (QW-852) | | 0.019 | 5114 | 269 |
| RSL3 | | 0.019 | 1.35 | 71 |
| 1 (VP-224) | SU-DHL-5 | 0.074 | 1.57 | 21 |
| 2 (QW-852) | | 9.520e−011 | 1.58 | >1000 |
| RSL3 | | 0.40 | 2.15 | 5 |
| 1 (VP-224) | SU-DHL-2 | 3.351e−012 | 0.69 | >1000 |
| 2 (QW-852) | | 0.002 | 4.95 | >1000 |
| RSL3 | | 0.18 | 1.50 | 8 |
| 1 (VP-224) | SU-DHL-7 | 0.026 | 13.92 | 535 |
| 2 (QW-852) | | 0.031 | >1000 | >1000 |
| RSL3 | | 0.17 | 1.00 | 6 |
| 1 (VP-224) | U-2932 | 0.30 | 13.24 | 44 |
| 2 (QW-852) | | 1.37 | 7.9 | 6 |
| RSL3 | | 0.16 | 316 | >1000 |

^{a}Selectivity (fold): IC₅₀ with Fer-1/IC₅₀. Data are presented as mean ± SEM of n = 3 technical replicates. Dose-response curves are shown in FIG. 102.

### VP-224 and QW-852 are Covalent GPX4 Inhibitors

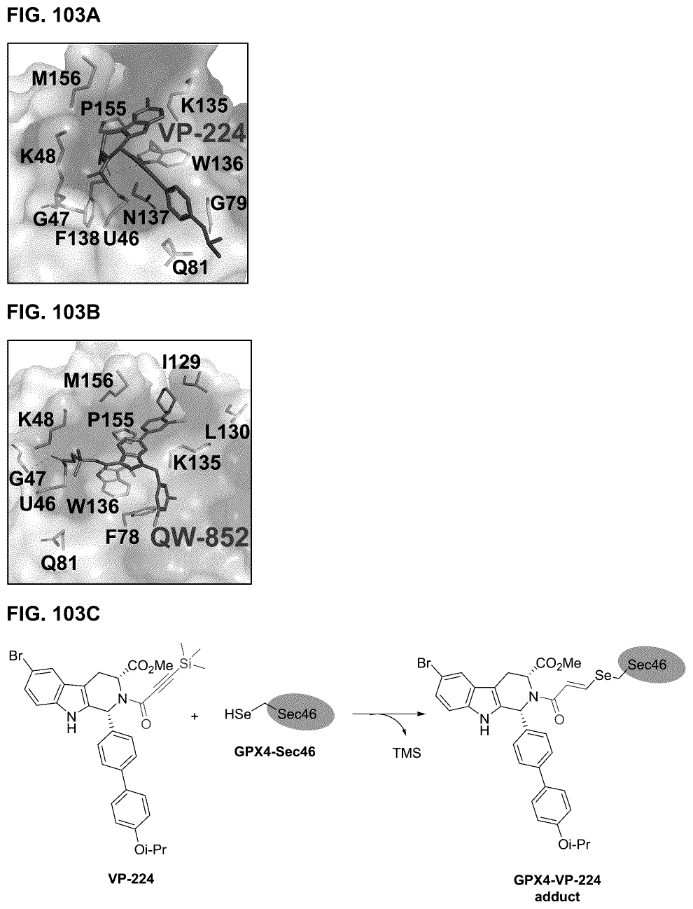

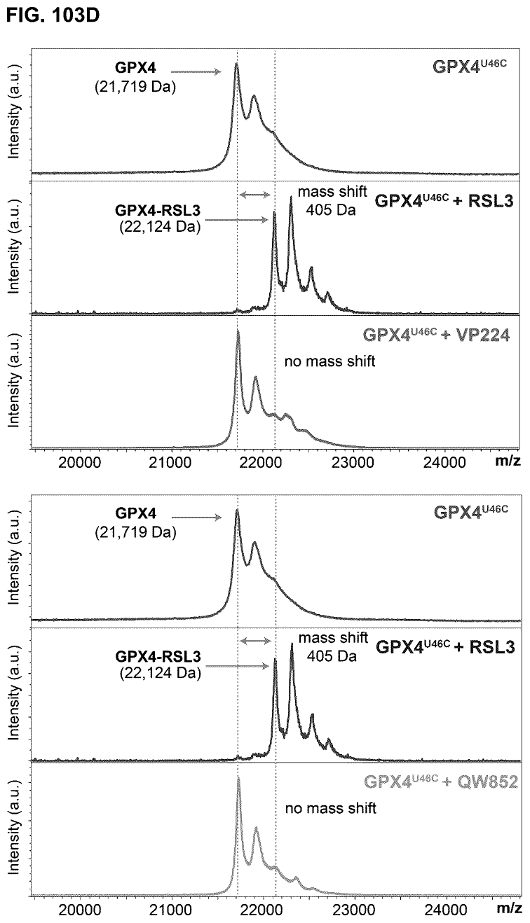

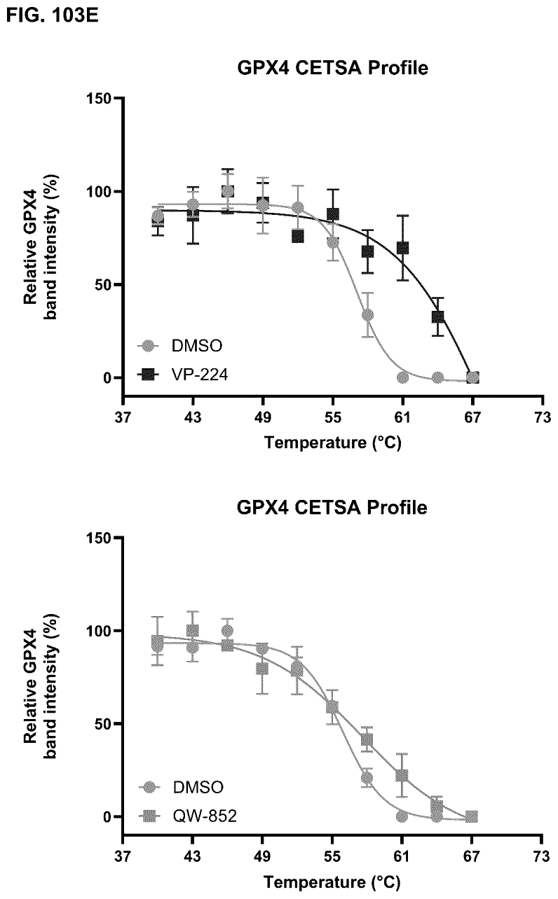

Building on the previous findings that VP-224 and QW-852 inhibit GPX4 activity in an enzymatic assay, molecular docking studies were conducted to gain deeper insights into their interactions with GPX4. Covalent docking of inhibitor QW-852 (deep purple for carbon atoms) onto the active site of WT GPX4 (PDB code: 6HKQ) showed that the inhibitor has one preferred pose that could presumably arise from presence of the di-methyl group (between the carbon and amide groups), which imposes that the major portion of the inhibitor to position itself in the shallow groove surrounded by predominantly hydrophobic residues (FIG. 103A). Consequently, the inhibitor has numerous interactions, primarily hydrophobic, with the residues near the active site of the enzyme. The inhibitor forms only one hydrogen bond (dash line) with the amide backbone of G47. Whereas the iso-indazole moiety forms π-π stacking with the indole ring of W136, the dichloro-benzyl group forms p-p interactions with both W136 and F78. The chloro-benzyl group forms hydrophobic interactions with P155 and carbon atoms of K135. Lastly, the morpholine moiety forms hydrophobic interaction with side chains of I129, L130, and M156, all of which are plausibly involved in binding to the cell membrane during catalysis (Labrecque and Fuglestad, 2021).

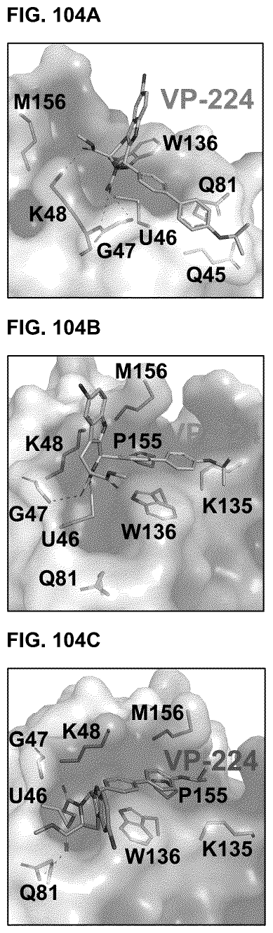

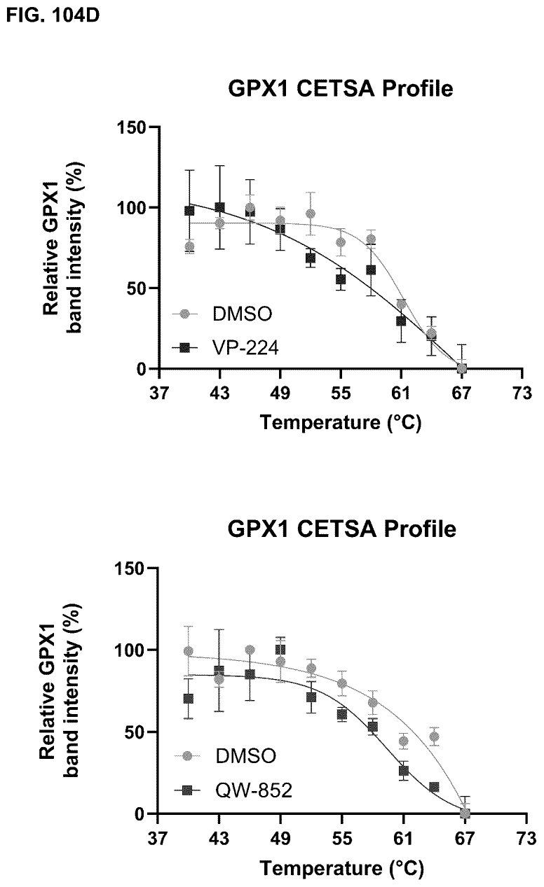

On the other hand, it has been revealed that VP-224 could have 4 isomers (FIG. 103B and FIGS. 104A-104C), presumably due to the chiral nitrogen of piperidine group. In FIG. 103B, where the best pose of the inhibitor is shown, VP-224 forms three hydrogen bonds with backbone amides of G47 and K48 and NH of the W136 indole group. While the bromo-indole group of VP-224 forms π-π stacking with the indole ring of W136, the benzyl group attached to the piperidine moiety forms π-π interactions with W136. Whereas the isopropyl phenyl ether group makes hydrophobic interactions with carbon atoms of G79 and Q81, the methyl ester group has hydrophobic interactions with both F138 and N137. These structural insights suggest that both VP-224 and QW-852 inhibit GPX4 through covalent interaction, likely by reacting with the selenocysteine residue (FIG. 103C).

To validate this mechanism of action (MoA), intact protein Matrix Assisted Laser Desorption/Ionization (MALDI) mass spectrometry (MS) analysis was performed. When VP-224 and QW-852 were tested in vitro by MALDI MS after pre-incubation with purified GPX4^{U46C} protein, where selenocysteine 46 is mutated to cysteine, both VP-224 and QW-852 exhibited lower potency than RSL3 to covalently bind to GPX4^{U46C} and shift its mass (FIG. 103D). Considering that both inhibitors have remarkably higher potency than RSL3 to bind to wild-type GPX4 in cell lysate to inhibit its activity in biochemical assay and that the previous study (Liu et al. 2022) demonstrated RSL3 specifically binds to Cysteine 66 of GPX4^{U46C}, we concluded that VP-224 and QW-852 primarily bind to the active site selenocysteine 46 of wild-type GPX4 in cancer cells. To further examine whether VP-224 and QW-852 directly bind to GPX4, a cellular thermal shift assay (CETSA) was performed (Jafari et al. 2014). CETSA results indicated that both VP-224 and QW-852 influence the thermal stability of GPX4 by stabilizing the protein (FIG. 103E). Unlike GPX4, the thermal stability of GPX1, another selenoprotein in the glutathione peroxidase family, was not affected (FIG. 104D).

### VP-224 and QW-852 Induce GPX4 Degradation Via the Ubiquitin-Proteasome System

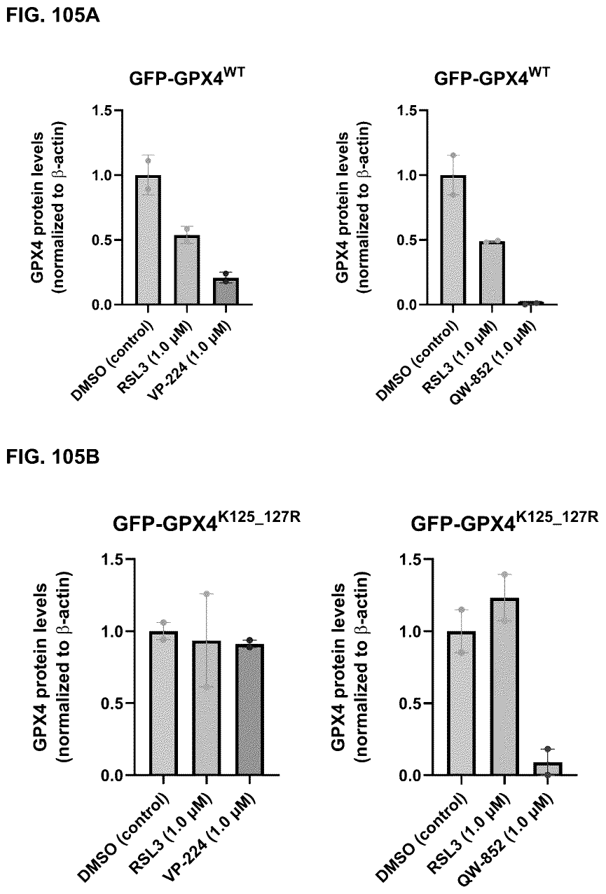

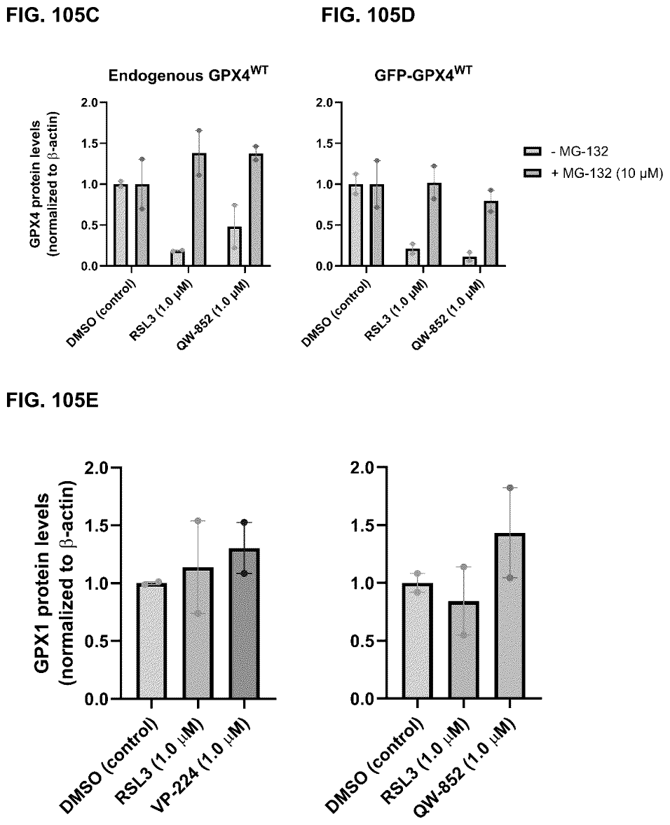

Eukaryotic cells degrade cellular proteins through two main pathways: the ubiquitin-proteasome system (UPS) and autophagy. Previously, it was reported that RSL3 might induce GPX4 degradation through a proteasome-dependent mechanism, with Lys125 and Lys127 identified as potential ubiquitination sites on GPX4 (Liu et al. 2022). To investigate whether VP-224 and QW-852 induce GPX4 degradation via a mechanism similar to RSL3, we examined GPX4 degradation in HT-1080 cells stably overexpressing GFP-GPX4^{K125R_K127R}, in which the proposed sites of ubiquitin ligation were removed by point mutations. It was found that GFP-GPX4^{K125R_K127R} was resistant to degradation by VP-224, but not by QW-852 (FIGS. 105A-105B). Next, we investigated the effect of co-treating HT-1080 cells overexpressing GFP-GPX4^{WT} with VP-224 or QW-852 alongside the proteasome inhibitor MG-132 on GPX4 degradation. Our data revealed that the degradation of GPX4 induced by VP-224 or QW-852 was effectively blocked by MG-132 (FIGS. 105C-105D). In contrast, we observed that both inhibitors were less effective at degrading GPX1, suggesting selectivity towards GPX4 protein (FIG. 105E).

### Evaluation of in Vitro and in Vivo Safety of VP-224 and QW-852

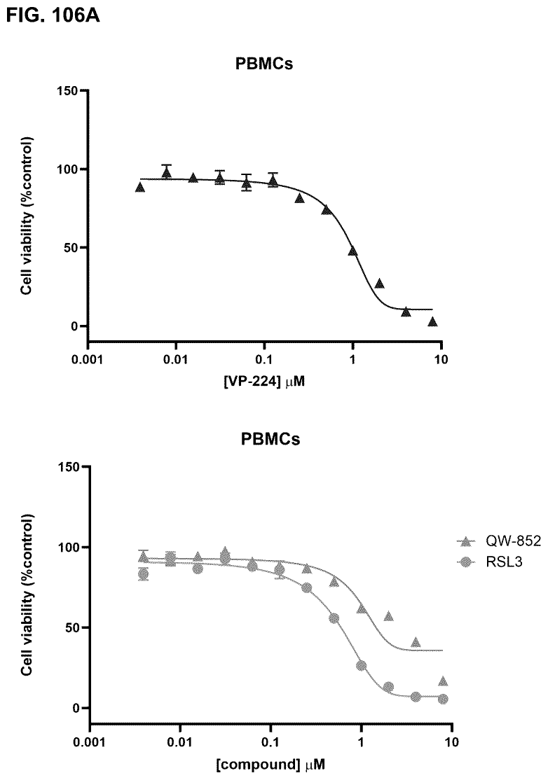

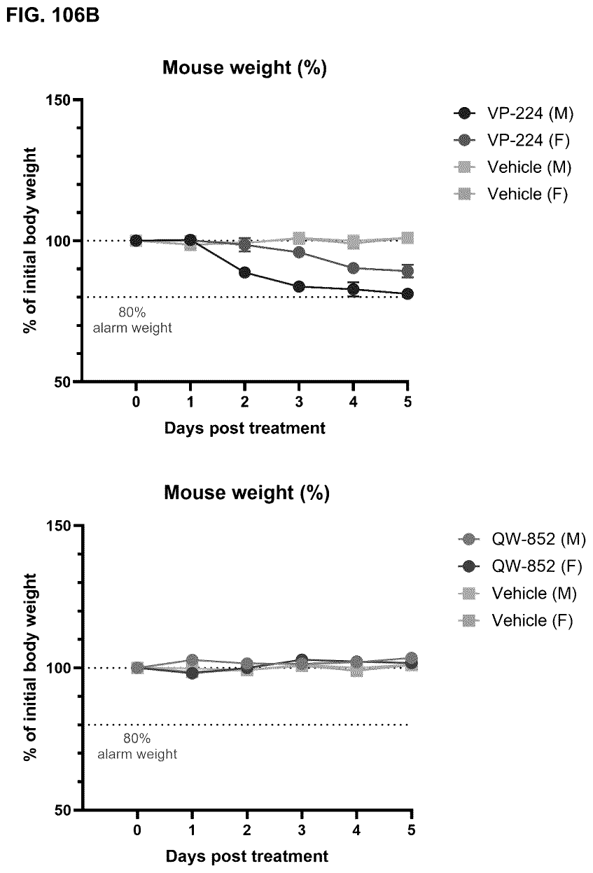

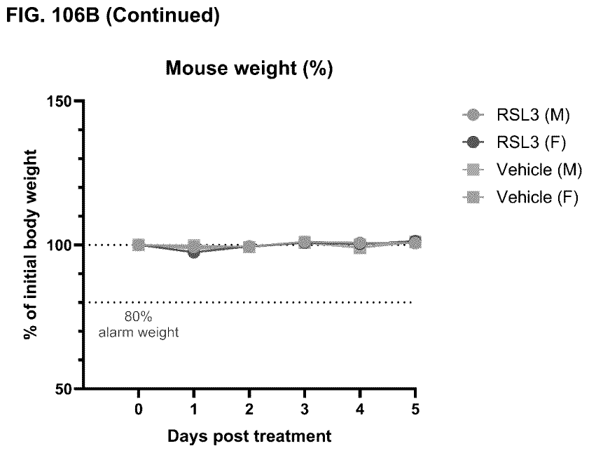

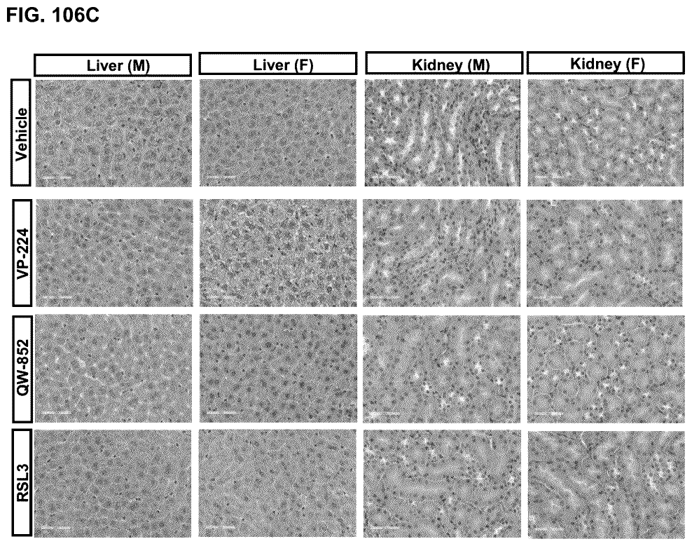

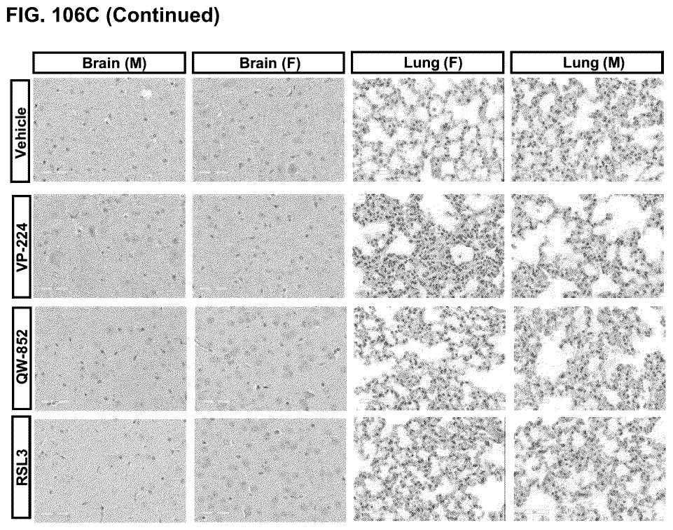

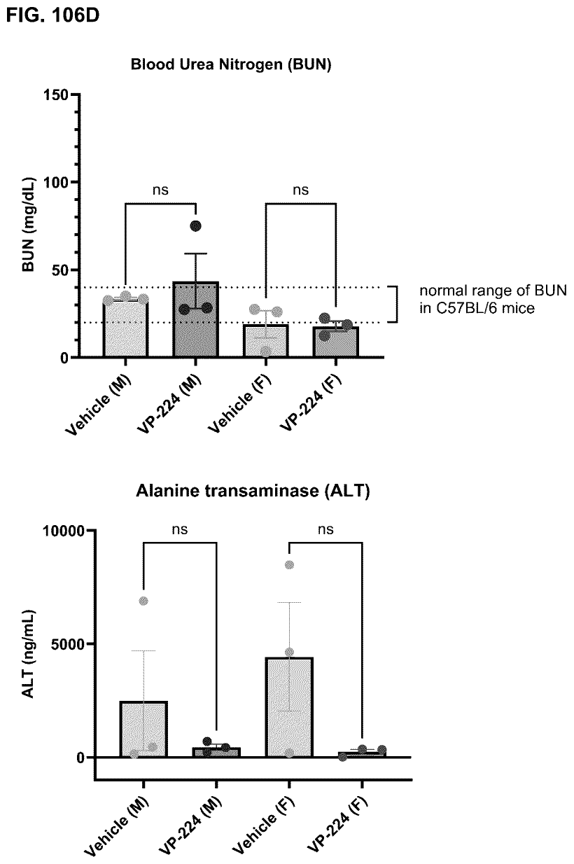

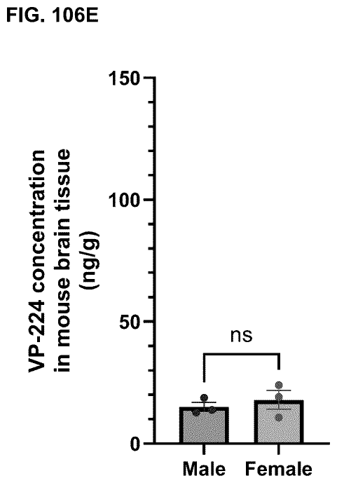

To evaluate the selectivity of VP-224 and QW-852 for sensitive tumor cells, such as HT-1080 and DLBCL cells, over normal cells, we assessed their cytotoxic effects on normal human peripheral blood mononuclear cells (PBMCs). Over 10-fold selectivity was observed for VP-224 and QW-852 in cancer cells compared to PBMCs, indicating a potential safety window (FIG. 106A). Furthermore, VP-224 was evaluated for its mutagenic potential using the Ames test, as compounds with mutagenic properties may contribute to carcinogenesis due to the strong link between genetic mutations and cancer. VP-224 tested negative in the Ames test, indicating no detectable mutagenic potential (Table 13). Additionally, VP-224 was screened against a panel of 44 critical molecular targets, including 24 GPCRs, 7 ion channels, 8 enzymes, 3 transporters, and 2 nuclear receptors. This comprehensive assessment aimed to identify potential off-target interactions for an early evaluation of its safety profile. VP-224 exhibited no significant off-target effects for the majority of the tested targets (Table 14).

TABLE 13
Mini-Ames genetic toxicity assay of VP-224. Mutagenicity potential evaluation using four Salmonella
typhimurium bacterial strains, including TA98, TA100, TA1535, and TA1537, in the presence and absence
of S9 liver fraction from Arochlor-induced rat. Weak positive, if p < 0.05, denoted as “+”; Strong
positive, if p < 0.01, denoted as “++”; Very strong positive, if p < 0.001, denoted as “+++”.

| | Test | Bacterial Strain (+/−S9) Positive Significance (− to +++) | | | | | | | |
|---|---|---|---|---|---|---|---|---|---|
| | Conc. | TA98 − | TA98 + | TA100 − | TA100 + | TA1535 − | TA1535 + | TA1537 − | TA1537 + |
| Compound ID | (μM) | S9 | S9 | S9 | S9 | S9 | S9 | S9 | S9 |
| VP-224 | 5.0 | − | − | − | − | − | − | − | − |
| | 10 | − | − | − | − | − | − | − | − |
| | 50 | − | − | − | − | − | − | − | − |
| | 100 | − | − | − | − | − | − | − | − |
| 2-Aminoanthracene | 1 | − | +++ | − | +++ | − | ++ | − | +++ |
| 2-Nitrofluorene | 30 | +++ | +++ | − | − | − | − | − | − |
| 9-Aminoacridine | 10 | − | − | − | − | − | − | +++ | ++ |
| Streptozotocin | 2.5 | + | − | +++ | +++ | +++ | +++ | − | − |

TABLE 14
Off-target screening of VP-224 for early safety pharmacology assessment. Compound was tested at a final assay concentration
of 10 μM in duplicate against 44 targets in the following target classes: GPCRs, ion channels, biogenic amine
transporters, nuclear receptors phosphodiesterases, cytochrome P450 isozymes, kinases and proteases.

| | | | | IC50 | | | | |
|---|---|---|---|---|---|---|---|---|
| | | | | (M) | | | Radiolig | |
| | | | | Reference | | | and | |
| | | | | Compound | IC50 | | (RL) | |
| | | % Activity | | (EC50 | (M) | | (all | RL |
| Target | | VP-224 | VP-224 | for | Reference | Reference | [3H]- | Conc. |
| Family | Target | Data 1 | Data 2 | agonist) | Compound | Compound | ligands) | (nM) |
| G-protein | 5-HT1A | 108 | 108 | 1.24E−08 | | 8-OH | 8-OH | 10 |
| Coupled | | | | | | DPAT | DPAT | |
| Receptors | 5-HT1B | 132 | 121 | 1.49E−08 | | GR125743 | GR125743 | 10 |
| (GPCRs) | 5-HT2A | 105 | 113 | 1.21E−09 | | Ketanserin | LSD | 2 |
| | 5-HT2B | 111 | 105 | 7.53E−09 | | Serotonin | LSD | 2 |
| | Adenosine | 108 | 99 | 1.36E−07 | | CGS212680 | CGS21680 | 15 |
| | A2A | | | | | | | |
| | Adrenergic | 124 | 113 | 1.24E−08 | | Prazosin | Prazosin | 10 |
| | a1A | | | | | | | |
| | Adrenergic | 97 | 104 | 8.51E−09 | | RX821002 | RX821002 | 5 |
| | a2A | | | | | | | |
| | Adrenergic b1 | 101 | 100 | 4.37E−08 | | Alprenolol | Alprenolol | 5 |
| | Adrenergic b2 | 92 | 90 | 6.62E−08 | | Alprenolol | Alprenolol | 15 |
| | Cannabinoid | 91 | 74 | 1.54E−08 | | CP55,940 | CP55940 | 1 |
| | CB1 | | | | | | | |
| | Cannabinoid | 31 | 30 | 1.06E−08 | | WIN55212-2 | CP55940 | 2 |
| | CB2 | | | | | | | |
| | CCK | 97 | 97 | 2.48E−07 | | Lorglumide | CCK | 5 |
| | Dopamine D1 | 104 | 111 | 5.66E−07 | | SCH23390 | SCH23390 | 3 |
| | Dopamine D2S | 63 | 60 | 2.67E−08 | | 7-OH | 7-OH | 15 |
| | | | | | | DPAT | DPAT | |
| | Endothelin | 0 | 0 | 9.32E−08 | | Endothelin | | |
| | ETA (Agonist) | | | | | | | |
| | Endothelin | 119 | 122 | 8.07E−08 | | Sitaxentin | | |
| | ETA | | | | | | | |
| | (Antagonist) | | | | | | | |
| | Histamine H1 | 89 | 80 | 2.56E−09 | | Mepyramine | Pyrilamine | 5 |
| | Histmaine H2 | 107 | 91 | 2.21E−08 | | Ranitidine | Famotidine | 20 |
| | Muscarinic M1 | 111 | 111 | 1.79E−08 | | Pirenzepine | Pirenzipine | 5 |
| | Muscarinic M2 | 80 | 101 | 1.93E−08 | | AF-DX384 | AF-DX384 | 5 |
| | Muscarinic M3 | 109 | 109 | 6.22E−09 | | 4-DAMP | NMS | 6 |
| | d opioid | 97 | 98 | 4.67E−08 | | DADLE | DADLE | 10 |
| | k opioid | 124 | 127 | 2.12E−07 | | U-69,593 | U69,593 | 15 |
| | m opioid | 106 | 91 | 4.04E−08 | | DAMGO | DAMGO | 15 |
| | V1A (Agonist)* | 0 | 0 | 3.32E−11 | | Vasopressin | | |
| | V1A | −2 | −2 | 1.27E−09 | | SR- | | |
| | (Antagonist)* | | | | | 49059 | | |
| Transporters | DAT | 143 | 120 | 2.26E−07 | | AHN 1-055 | WIN35428 | 15 |
| | NET | 121 | 115 | 6.98E−09 | | Nisoxetine | Nisoxetine | 4 |
| | SERT | 407 | 498 | 3.85E−08 | | Imipramine | Imipramine | 20 |
| Ion | 5HT3 | 96 | 102 | 1.12E−08 | | Quipazine | Ondansetron | 20 |
| Channels | Central BZD | 104 | 100 | 1.70E−08 | | Flumazenil | Ro151788 | 10 |
| | CaV1.2 | 104 | 106 | 6.33E−08 | | Nifedipine | | |
| | hERG | 117 | 116 | 4.00E−08 | | E-4031 | | |
| | NaV1.5 | 110 | 105 | 1.84E−06 | | TTX | | |
| | nAChR | −6 | −7 | 2.50E−09 | | Epibatidine | | |
| | (Agonist) | | | | | | | |
| | nAChR | 107 | 102 | 6.80E−07 | 8.31E−07 | Mecamylamine | | |
| | (Antagonist) | | | | | | | |
| | NMDA | 93 | 97 | 2.75E−08 | | MK-801 | | |
| Kinase | Lck | 96 | 92 | 2.06E−09 | | Staurosporine | | |
| Phosphodiesterase | PDE3A | 99 | 103 | 5.22E−06 | | IBMX | | |
| | PDE4D2 | 108 | 105 | 7.74E−06 | | IBMX | | |
| Cyclooxygenases | COX-1 | 95 | 99 | 3.41E−08 | | SC-560 | | |
| | COX-2 | 97 | 98 | 4.53E−08 | | DuP-697 | | |
| Cholinesterase | Acetylcholinesterase | 120 | 121 | 1.05E−07 | | Physostigmine | | |
| Monoamine | MAO-A | 90 | 75 | 1.45E−07 | | Tranylcypromine | | |
| Oxidase | MAO-B | 81 | 75 | 9.21E−08 | | Tranylcypromine | | |
| Nuclear | Androgen | −18 | −18 | 1.37E−10 | | 5aDH-11- | | |
| | Receptor | | | | | KT | | |
| | (Agonist) | | | | | | | |
| Receptors | Androgen | −36 | −36 | 3.24E−08 | 4.88E−08 | Apalutamide | | |
| | Receptor | | | | | | | |
| | (Antagonist)* | | | | | | | |
| | Glucocorticoid | 102 | 126 | 9.62E−09 | | Flucatisone | | |
| | Receptor | | | | | | | |
| | (Agonist) | | | | | | | |

*Cell Reporter assays: in presence of VP-224, luminescence below background, similar to values without cells - possible cytotoxic effect.

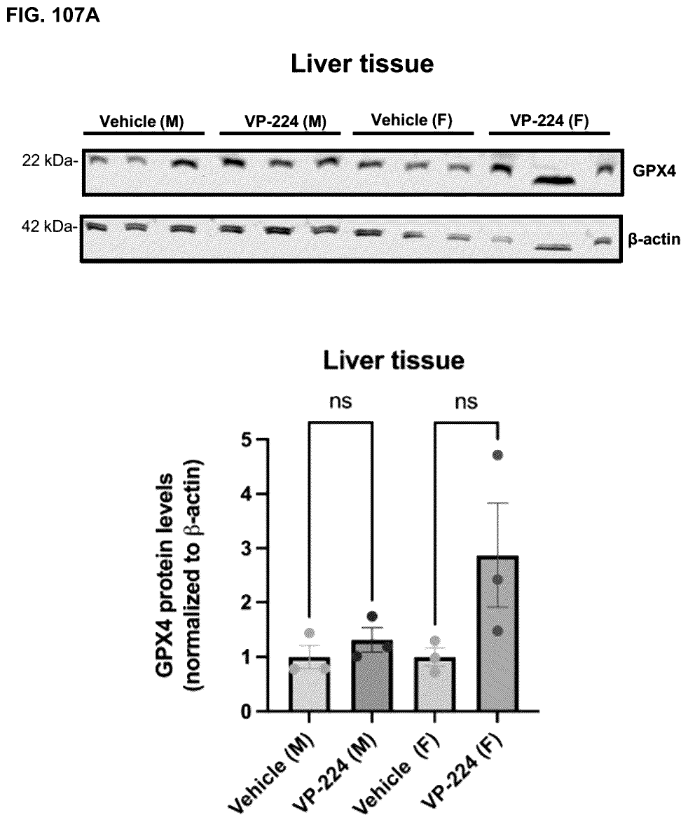

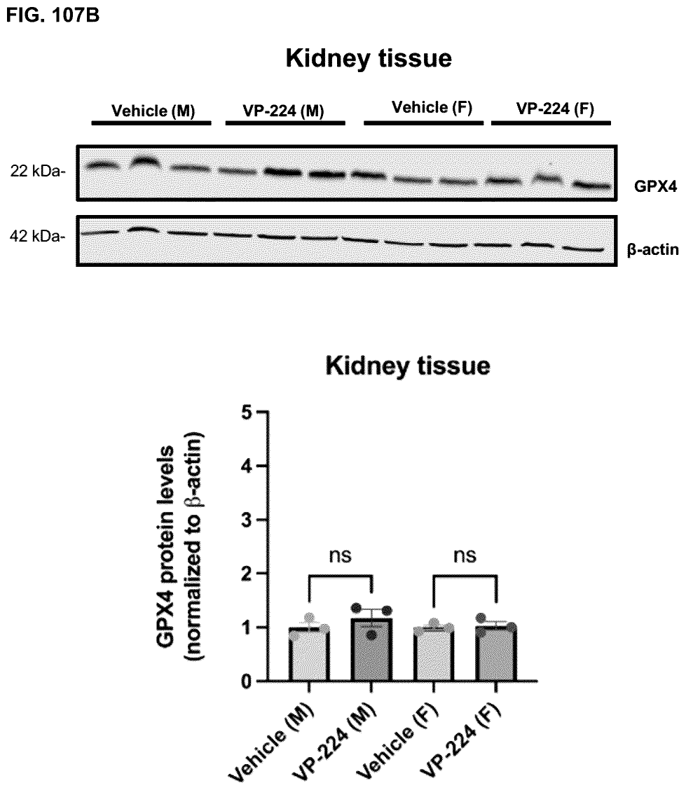

To assess the in vivo safety of the new GPX4 inhibitors, VP-224 and QW-852, a cohort of healthy C57BL/6 mice (n=6, 3 males and 3 females) was randomly assigned to one of the following groups: control (vehicle), VP-224-treated (20 mg/kg, IP), QW-852-treated (20 mg/kg, IP), and RSL3-treated (20 mg/kg, IP) groups. Mice were dosed once daily for 5 consecutive days. None of the tested groups experienced mortality during the treatment period. However, the VP-224-treated group exhibited nearly 20% weight loss, whereas the RSL3- and QW-852-treated groups maintained their weight throughout the treatment period (FIG. 106B). Notably, female mice appeared to tolerate the VP-224 treatment better than males. To further assess the safety of VP-224 and QW-852, H&E staining analysis of major organs was performed. No significant differences were observed across treatment groups in either sex, and the cellular and tissue morphology remained unaffected. (FIG. 106C). Common biochemical indicators of hepatorenal function, such as BUN and ALT, were measured in the VP-224-treated group, showing no significant differences compared to the control group (FIG. 106D). Additionally, GPX4 expression in liver and kidney tissues was assessed by western blot, revealing no significant differences between the VP-224-treated and control groups (FIGS. 107A-107B). Furthermore, VP-224 concentration in brain tissue was determined, with a final concentration of approximately 16 ng/g (FIG. 106E), suggesting limited brain penetration and a reduced risk of CNS toxicity.

### DOCUMENTS CITED

1. Adams, P. D. et al. PHENIX: a comprehensive Python-based system for macromolecular structure solution. Acta Crystallogr D Biol Crystallogr 66, 213-221 (2010).
2. Agmon E, Solon J, Bassereau P, Stockwell B R. Modeling the effects of lipid peroxidation during ferroptosis on membrane properties. Sci Rep. 2018; 8(1):5155. Epub 2018 Mar. 28. doi: 10.1038/s41598-018-23408-0. PubMed PMID: 29581451; PMCID: PMC5979948.
3. Agmon, E. & Stockwell, B. R. Lipid homeostasis and regulated cell death. Curr Opin Chem Biol 39, 83-89, doi:10.1016/j.cbpa.2017.06.002 (2017).
4. Baker, N. A., Sept, D., Joseph, S., Holst, M. J. & McCammon, J. A. Electrostatics of nanosystems: Application to microtubules and the ribosome. Proceedings of the National Academy of Sciences 98, 10037-10041 (2001).
5. Dixon S J, Stockwell B R. The Hallmarks of Ferroptosis. Annual Review of Cancer Biology. 2019; 3:35-54.
6. Dixon, S. J. et al. Ferroptosis: an iron-dependent form of nonapoptotic cell death. Cell 149, 1060-1072, doi:10.1016/j.cell.2012.03.042 (2012).
7. Eaton, J. K., Furst, L., Cai, L. L., Viswanathan, V. S., and Schreiber, S. L. (2020a). Structure-activity relationships of GPX4 inhibitor warheads. Bioorg Med Chem Lett 30, 127538. 10.1016/j.bmcl.2020.127538.
8. Eaton, J. K., Furst, L., Ruberto, R. A., Moosmayer, D., Hilpmann, A., Ryan, M. J., Zimmermann, K., Cai, L. L., Niehues, M., Badock, V., et al. (2020b). Selective covalent targeting of GPX4 using masked nitrile-oxide electrophiles. Nat Chem Biol 16, 497-506. 10.1038/s41589-020-0501-5.
9. Eaton, J. K., Ruberto, R. A., Kramm, A., Viswanathan, V. S., and Schreiber, S. L. (2019). Diacylfuroxans Are Masked Nitrile Oxides That Inhibit GPX4 Covalently. J Am Chem Soc 141, 20407-20415. 10.1021/jacs.9b10769.
10. EMMA LACHAIER, C. L., CORINNE GODIN, ZUZANA SAIDAK, and MAXIME BAERT, M. D., BRUNO CHAUFFERT and ANTOINE GALMICHE (2014). Sorafenib Induces Ferroptosis in Human Cancer Cell Lines Originating from Different Solid Tumors. ANTICANCER RESEARCH 34, 6417-6422.
11. Emsley, P., Lohkamp, B., Scott, W. G. & Cowtan, K. Features and development of Coot. Acta Crystallogr D Biol Crystallogr 66, 486-501 (2010).
12. Feng, H. et al. K-RasG12D has an allosteric small molecule binding site. Biochemistry 58, 2542-2554 (2019).
13. Hangauer, M. J. et al. Drug-tolerant persister cancer cells are vulnerable to GPX4 inhibition. Nature 551, 247-250, doi:10.1038/nature24297 (2017).
14. Jafari R, Almqvist H, Axelsson H, Ignatushchenko M, Lundbäck T, Nordlund P, Molina D M. The cellular thermal shift assay for evaluating drug target interactions in cells. Nature Protocols. 2014; 9(9):2100-22. doi: 10.1038/nprot.2014.138.
15. Kabsch, W. Integration, scaling, space-group assignment and post-refinement. Acta Crystallogr D Biol Crystallogr 66, 133-144 (2010).
16. Labrecque C L, Fuglestad B. Electrostatic Drivers of GPx4 Interactions with Membrane, Lipids, and DNA. Biochemistry. 2021; 60(37):2761-72. doi: 10.1021/acs.biochem.1c00492.
17. Liangyu Chen, X.n.L., Lib o Liu, Bo Yu, Yix ue Xue and Yunhui Liu (2015). Erastin sensitizes glioblastoma cells to temozolomide by restraining xCT and cystathionine-γ-lyase function. ONCOLOGY REPORTS 33, 1465-1474. 10.3892/or.2015.3712.
18. Liu H, Forouhar F, Lin A J, Wang Q, Polychronidou V, Soni R K, Xia X, Stockwell B R. Small-molecule allosteric inhibitors of GPX4. Cell Chem Biol. 2022; 29(12):1680-93.e9.
19. Liu H, Forouhar F, Seibt T, Saneto R, Wigby K, Friedman J, Xia X, Shchepinov M S, Ramesh S K, Conrad M, Stockwell B R. Characterization of a patient-derived variant of GPX4 for precision therapy. Nat Chem Biol. 2022; 18(1):91-100. doi: 10.1038/s41589-021-00915-2.
20. Lo, M., Ling, V., Wang, Y. Z., and Gout, P. W. (2008). The xc-cystine/glutamate antiporter: a mediator of pancreatic cancer growth with a role in drug resistance. Br J Cancer 99, 464-472. 10.1038/sj.bjc.6604485.
21. Luft, J. R. et al. A deliberate approach to screening for initial crystallization conditions of biological macromolecules. Journal of Structural Biology 142, 170-179 (2003).
22. Martinez, A. M., Kim, A. & Yang, W. S. Detection of Ferroptosis by BODIPY™ 581/591 C11. in Immune Mediators in Cancer: Methods and Protocols (eds. Vancurova, I. & Zhu, Y.) 125-130 (Springer US, 2020). doi:10.1007/978-1-0716-0247-8_11.
23. McRee, D. E. XtalView/Xfit—A Versatile Program for Manipulating Atomic Coordinates and Electron Density. Journal of Structural Biology 125, 156-165 (1999).
24. Moosmayer, D., Hilpmann, A., Hoffmann, J., Schnirch, L., Zimmermann, K., Badock, V., Furst, L., Eaton, J. K., Viswanathan, V. S., Schreiber, S. L., et al. (2021). Crystal structures of the selenoprotein glutathione peroxidase 4 in its apo form and in complex with the covalently bound inhibitor ML162. Acta Crystallogr D Struct Biol 77, 237-248. 10.1107/S2059798320016125.
25. Patrick Scheerer, A. B., Norbert Krauss, Helga Wessner, Christa Gerth, Wolfgang Hohne, and Hartmut Kuhn (2007). Structural Basis for Catalytic Activity and Enzyme Polymerization of Phospholipid Hydroperoxide Glutathione Peroxidase-4 (GPx4). Biochemistry 46, 9041-9049. 10.1021/bi700840d.
26. Roh, J. L., Kim, E. H., Jang, H. J., Park, J. Y., and Shin, D. (2016). Induction of ferroptotic cell death for overcoming cisplatin resistance of head and neck cancer. Cancer Lett 381, 96-103. 10.1016/j.canlet.2016.07.035.
27. Roveri, A., Maiorino, M. & Ursini, F. [20] Enzymatic and immunological measurements of soluble and membrane-bound phospholipid-hydroperoxide glutathione peroxidase. in Methods in Enzymology vol. 233 202-212 (Academic Press, 1994).
28. Scheerer, P. et al. Structural basis for catalytic activity and enzyme polymerization of phospholipid hydroperoxide glutathione peroxidase-4 (GPx4). Biochemistry 46, 9041-9049 (2007).
29. Stockwell B R, et al. Ferroptosis: A Regulated Cell Death Nexus Linking Metabolism, Redox Biology, and Disease. Cell. 2017; 171(2):273-85. Epub 2017 Oct. 7. doi: 10.1016/j.cell.2017.09.021. PubMed PMID: 28985560; PMCID: PMC5685180.
30. Stolwijk, J. M. et al. Simultaneous detection of the enzyme activities of GPx1 and GPx4 guide optimization of selenium in cell biological experiments. Redox Biology 32, 101518 (2020).
31. Vagin, A. & Teplyakov, A. Molecular replacement with MOLREP. Acta Cryst D 66, 22-25 (2010).
32. Viswanathan, V. S. et al. Dependency of a therapy-resistant state of cancer cells on a lipid peroxidase pathway. Nature 547, 453-457, doi:10.1038/nature23007 (2017).
33. Wang, W., Green, M., Choi, J. E., Gijon, M., Kennedy, P. D., Johnson, J. K., Liao, P., Lang, X., Kryczek, I., Sell, A., et al. (2019). CD8 (+) T cells regulate tumour ferroptosis during cancer immunotherapy. Nature 569, 270-274. 10.1038/s41586-019-1170-y.
34. Xu, C., Xiao, Z., Wang, J., Lai, H., Zhang, T., Guan, Z., Xia, M., Chen, M., Ren, L., He, Y., et al. (2021). Discovery of a Potent Glutathione Peroxidase 4 Inhibitor as a Selective Ferroptosis Inducer. J Med Chem 64, 13312-13326. 10.1021/acs.jmedchem.1c00569.
35. Yang, W. S. et al. Peroxidation of polyunsaturated fatty acids by lipoxygenases drives ferroptosis. Proc Natl Acad Sci USA 113, E4966-4975, doi:10.1073/pnas. 1603244113 (2016).
36. Yang, W. S. et al. Regulation of ferroptotic cancer cell death by GPX4. Cell 156, 317-331, doi:10.1016/j.cell.2013.12.010 (2014).
37. Yang, W. S., and Stockwell, B. R. (2008). Synthetic lethal screening identifies compounds activating iron-dependent, nonapoptotic cell death in oncogenic-RAS-harboring cancer cells. Chem Biol 15, 234-245. 10.1016/j.chembiol.2008.02.010.
38. Ye, L. F., Chaudhary, K. R., Zandkarimi, F., Harken, A. D., Kinslow, C. J., Upadhyayula, P. S., Dovas, A., Higgins, D. M., Tan, H., Zhang, Y., et al. (2020). Radiation-Induced Lipid Peroxidation Triggers Ferroptosis and Synergizes with Ferroptosis Inducers. ACS Chem Biol 15 469-484. 10.1021/acschembio.9b00939.
39. Yu, Y., Xie, Y., Cao, L., Yang, L., Yang, M., Lotze, M. T., Zeh, H. J., Kang, R., and Tang, D. (2015). The ferroptosis inducer erastin enhances sensitivity of acute myeloid leukemia cells to chemotherapeutic agents. Mol Cell Oncol 2, e1054549. 10.1080/23723556.2015.1054549.
40. Zhang Y, Tan H, Daniels J D, Zandkarimi F, Liu H, Brown L M, Uchida K, O'Connor O A, Stockwell B R. Imidazole Ketone Erastin Induces Ferroptosis and Slows Tumor Growth in a Mouse Lymphoma Model. Cell Chem Biol. 2019; 26(5):623-33.e9.
41. Zou, Y., Palte, M. J., Deik, A. A., Li, H., Eaton, J. K., Wang, W., Tseng, Y. Y., Deasy, R., Kost-Alimova, M., Dancik, V., et al. (2019). A GPX4-dependent cancer cell state underlies the clear-cell morphology and confers sensitivity to ferroptosis. Nat Commun 10, 1617. 10.1038/s41467-019-09277-9.

All documents cited in this application are hereby incorporated by reference as if recited in full herein.

Although illustrative embodiments of the present disclosure have been described herein, it should be understood that the disclosure is not limited to those described, and that various other changes or modifications may be made by one skilled in the art without departing from the scope or spirit of the disclosure.

What is claimed is:

1. A compound according to formula (1): 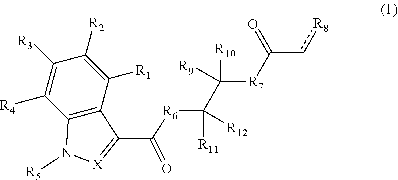
wherein:
a dashed line indicates the presence of an optional double bond;
X is selected from the group consisting of O, N, S, and C;
R₁, R₂, R₃, R₄ and R₅ are independently selected from the group consisting of H, D, —OH, halo, ether, ester, furanyl, indole, indazole, pyrrole, pyrazole, pyridine, pyrimidine, naphthalene, indene, dibenzofuran, benzodioxole, amide, —(O)C(R), —C(O)OR, —NO₂, alkyl, aryl, alkyl-aryl, alkyl-heteroaryl, alkenyl, alkenyl-aryl, alkenyl-heteroaryl, peptide, and polypeptide, wherein the ether, ester, furanyl, indole, indazole, pyrrole, pyrazole, pyridine, pyrimidine, naphthalene, indene, dibenzofuran, benzodioxole, amide, alkyl, aryl, alkyl-aryl, alkyl-heteroaryl, alkenyl, alkenyl-aryl, alkenyl-heteroaryl, peptide, and polypeptide may be optionally substituted with an atom or a group selected from the group consisting of O, N, S, epoxy, —OH, halo, —CN, —NO₂, C₁₋₄alkyl, CF₃, —CF₃O, carbonyl, alkyl-aryl, thiol, methylmethanesulfonamide, methylthiol, dioxane, tetrahydropyzan, morpholinyl, piperazine, sulfonyl, oxazole, oxadiazole, dizaole, triazole, amide, ether, ester and combinations thereof; or R₁ and R₂ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₂ and R₃ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₃ and R₄ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof,
R₆ and R₇ are independently selected from the group consisting of C, N, O and S; or R₆ and R₇ may together form a saturated or unsaturated ring structure that may be optionally substituted with an atom or a group from the group consisting of O, N, halo, C₁₋₄alkyl, CF₃, oxazole, oxadiazole, dizaole, triazole, amide, ether, ester and combinations thereof,
R₈ is selected from the group consisting of H, D, C, O, N, alkenyl, alkynyl, ester, oxazole, piperazine, morpholinyl and halo,
R₉, R₁₀, R₁₁ and R₁₂ are independently selected from the group consisting of no atom, H, D, —OH, halo, —CN, —NO₂, C₁₋₄alkyl, CF₃, —CF₃O, carbonyl, aryl, alkyl-aryl, thiol, ether, and ester,
wherein R is selected from the group consisting of H, D, O, N, halo, oxazole, oxadiazole, dizaole, triazole, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of O, N, S, halo, C₁₋₄alkyl, CF₃, —CN and combinations thereof,
or an N-oxide, crystalline form, hydrate, or pharmaceutically acceptable salt thereof.

2. The compound according to claim 1 having a structure selected from the group consisting of: 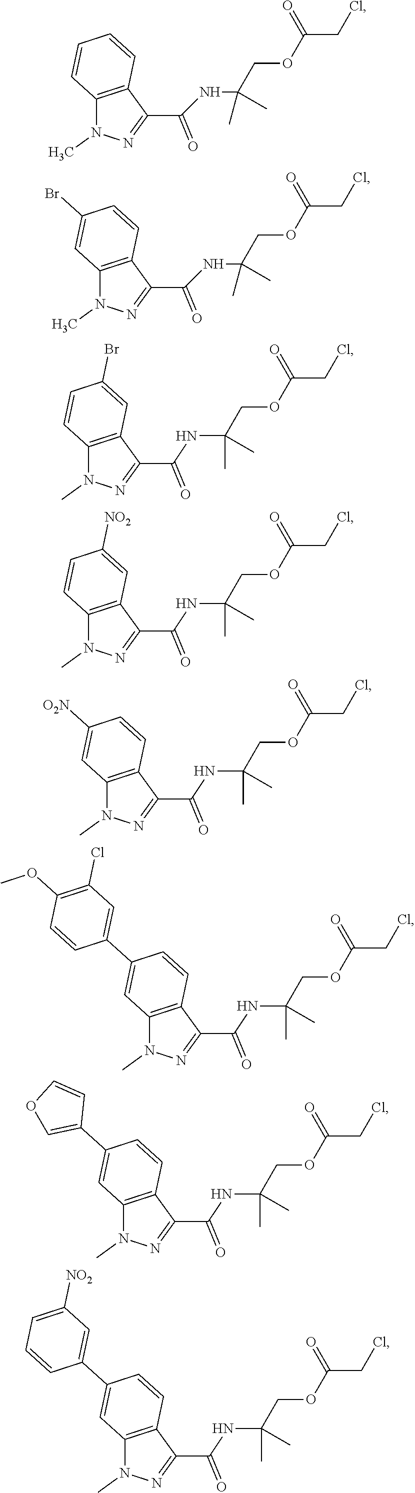 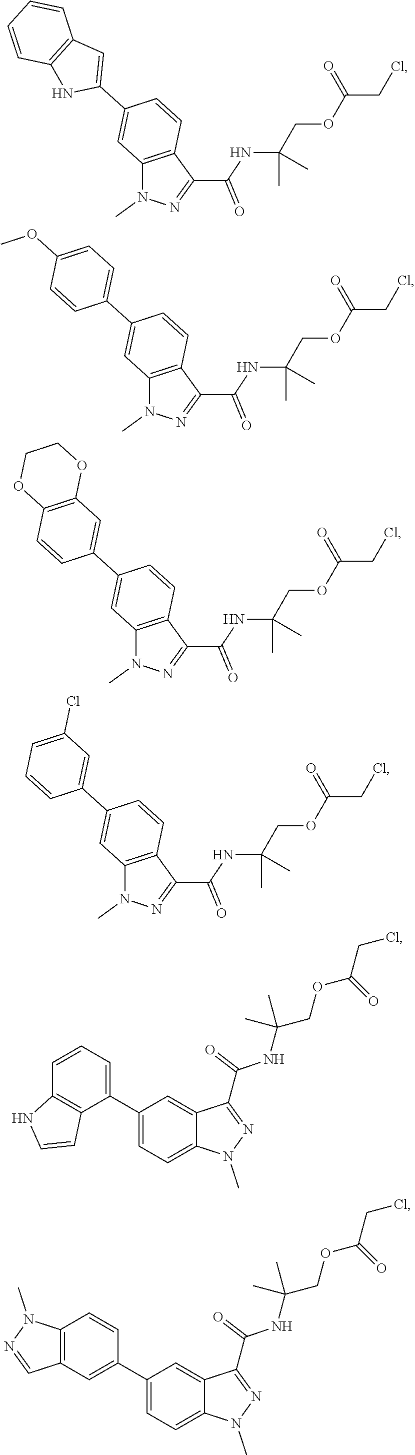 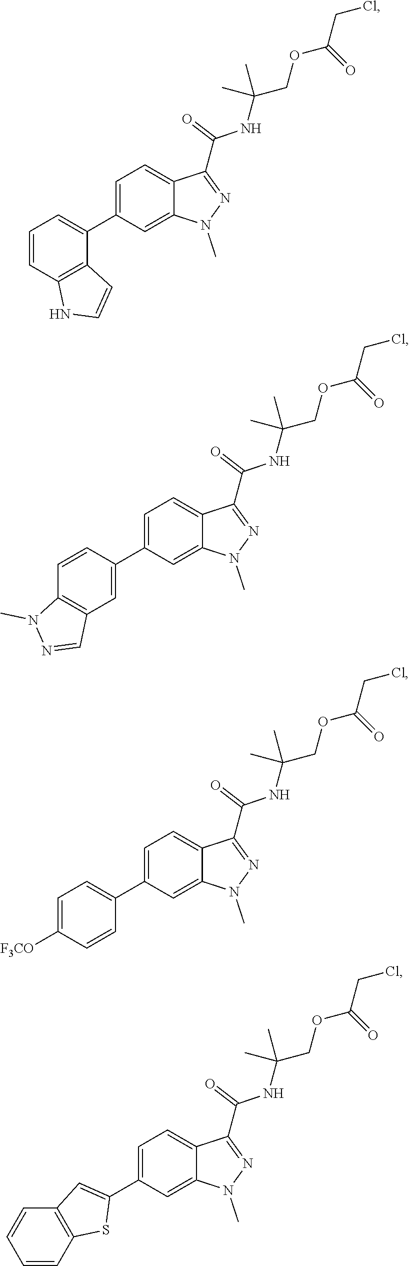 [SMILES: Cn1nc(C(=O)NC(C)(C)COC(=O)CCl)c2ccc(-c3cccc4[nH]ccc34)cc21 ; Cn1ncc2cc(-c3ccc4c(C(=O)NC(C)(C)COC(=O)CCl)nn(C)c4c3)ccc21 ; Cn1nc(C(=O)NC(C)(C)COC(=O)CCl)c2ccc(-c3ccc(OC(F)(F)F)cc3)cc21 ; Cn1nc(C(=O)NC(C)(C)COC(=O)CCl)c2ccc(-c3cc4ccccc4s3)cc21] 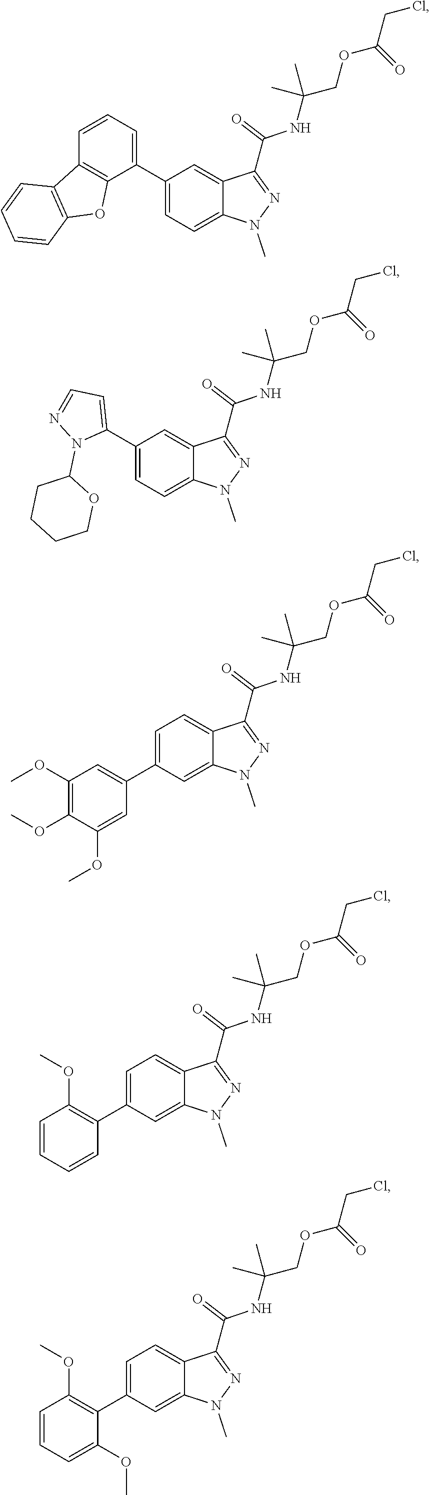 [SMILES: Cn1nc(C(=O)NC(C)(C)COC(=O)CCl)c2cc(-c3cccc4c3oc3ccccc34)ccc21 ; Cn1nc(C(=O)NC(C)(C)COC(=O)CCl)c2cc(-c3ccnn3C3CCCCO3)ccc21 ; COc1cc(-c2ccc3c(C(=O)NC(C)(C)COC(=O)CCl)nn(C)c3c2)cc(OC)c1OC ; COc1ccccc1-c1ccc2c(C(=O)NC(C)(C)COC(=O)CCl)nn(C)c2c1 ; COc1cccc(OC)c1-c1ccc2c(C(=O)NC(C)(C)COC(=O)CCl)nn(C)c2c1] 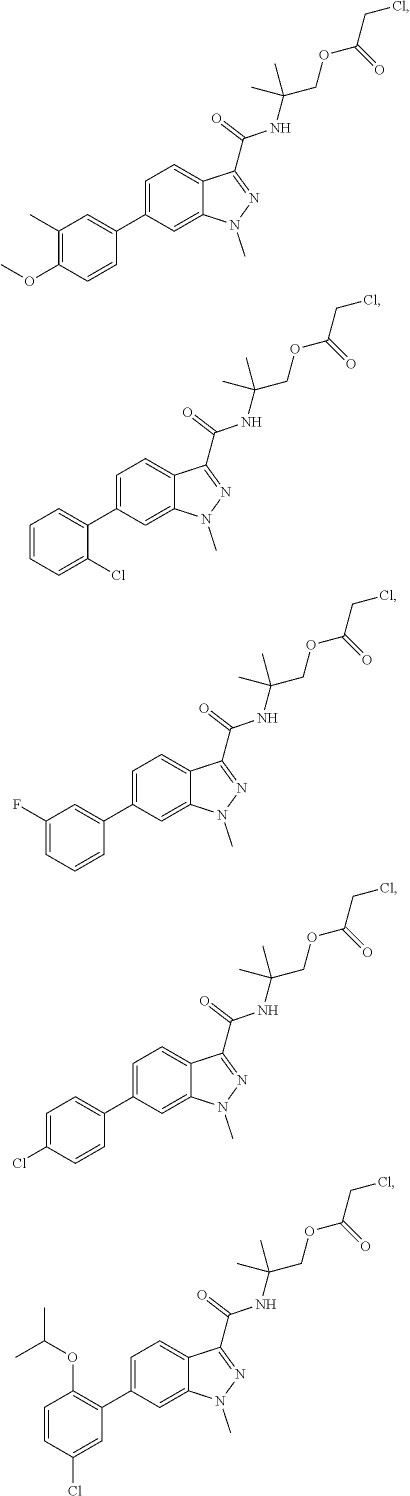 [SMILES: COc1ccc(-c2ccc3c(C(=O)NC(C)(C)COC(=O)CCl)nn(C)c3c2)cc1C ; Cn1nc(C(=O)NC(C)(C)COC(=O)CCl)c2ccc(-c3ccccc3Cl)cc21 ; Cn1nc(C(=O)NC(C)(C)COC(=O)CCl)c2ccc(-c3cccc(F)c3)cc21 ; Cn1nc(C(=O)NC(C)(C)COC(=O)CCl)c2ccc(-c3ccc(Cl)cc3)cc21 ; CC(C)Oc1ccc(Cl)cc1-c1ccc2c(C(=O)NC(C)(C)COC(=O)CCl)nn(C)c2c1] 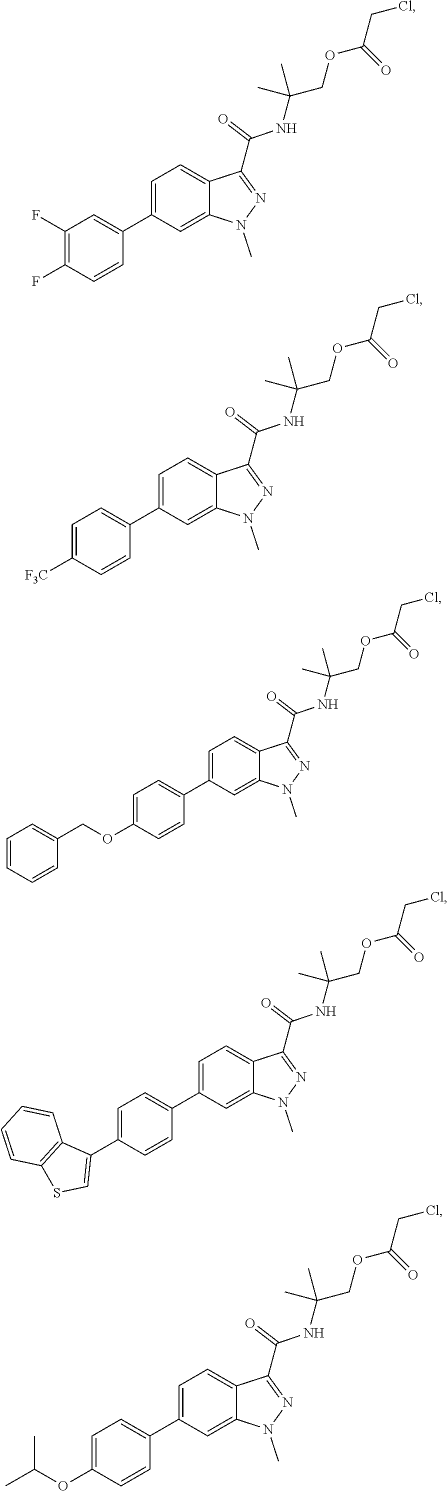 [SMILES: Cn1nc(C(=O)NC(C)(C)COC(=O)CCl)c2ccc(-c3ccc(F)c(F)c3)cc21 ; Cn1nc(C(=O)NC(C)(C)COC(=O)CCl)c2ccc(-c3ccc(C(F)(F)F)cc3)cc21 ; Cn1nc(C(=O)NC(C)(C)COC(=O)CCl)c2ccc(-c3ccc(OCc4ccccc4)cc3)cc21 ; Cn1nc(C(=O)NC(C)(C)COC(=O)CCl)c2ccc(-c3ccc(-c4csc5ccccc45)cc3)cc21 ; CC(C)Oc1ccc(-c2ccc3c(C(=O)NC(C)(C)COC(=O)CCl)nn(C)c3c2)cc1] 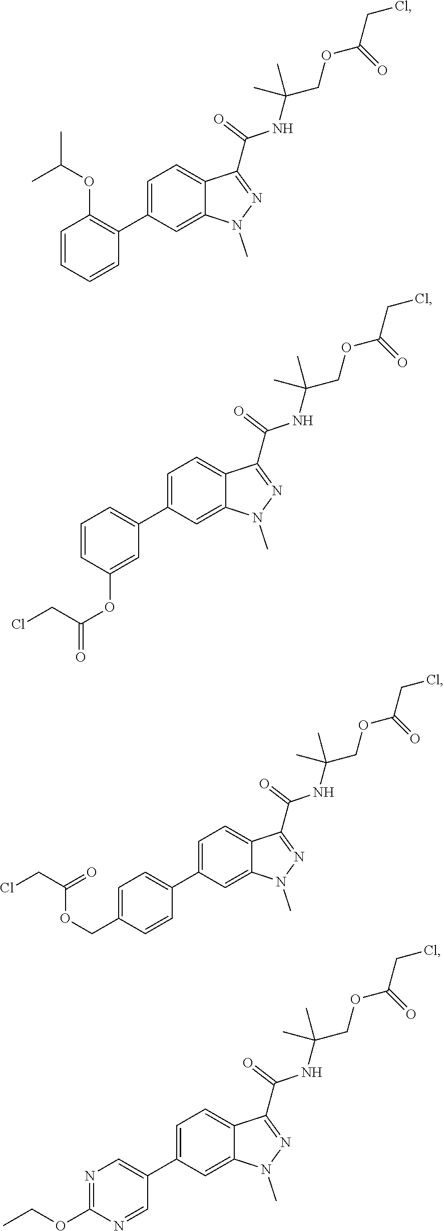 [SMILES: CC(C)Oc1ccccc1-c1ccc2c(C(=O)NC(C)(C)COC(=O)CCl)nn(C)c2c1 ; Cn1nc(C(=O)NC(C)(C)COC(=O)CCl)c2ccc(-c3cccc(OC(=O)CCl)c3)cc21 ; Cn1nc(C(=O)NC(C)(C)COC(=O)CCl)c2ccc(-c3ccc(COC(=O)CCl)cc3)cc21 ; CCOc1ncc(-c2ccc3c(C(=O)NC(C)(C)COC(=O)CCl)nn(C)c3c2)cn1] 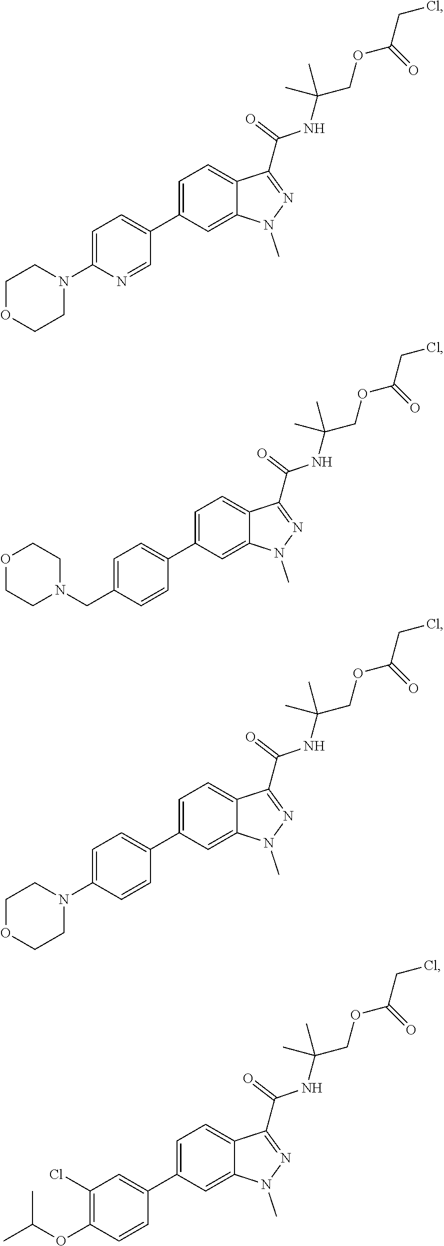 [SMILES: Cn1nc(C(=O)NC(C)(C)COC(=O)CCl)c2ccc(-c3ccc(N4CCOCC4)nc3)cc21 ; Cn1nc(C(=O)NC(C)(C)COC(=O)CCl)c2ccc(-c3ccc(CN4CCOCC4)cc3)cc21 ; Cn1nc(C(=O)NC(C)(C)COC(=O)CCl)c2ccc(-c3ccc(N4CCOCC4)cc3)cc21 ; CC(C)Oc1ccc(-c2ccc3c(C(=O)NC(C)(C)COC(=O)CCl)nn(C)c3c2)cc1Cl] 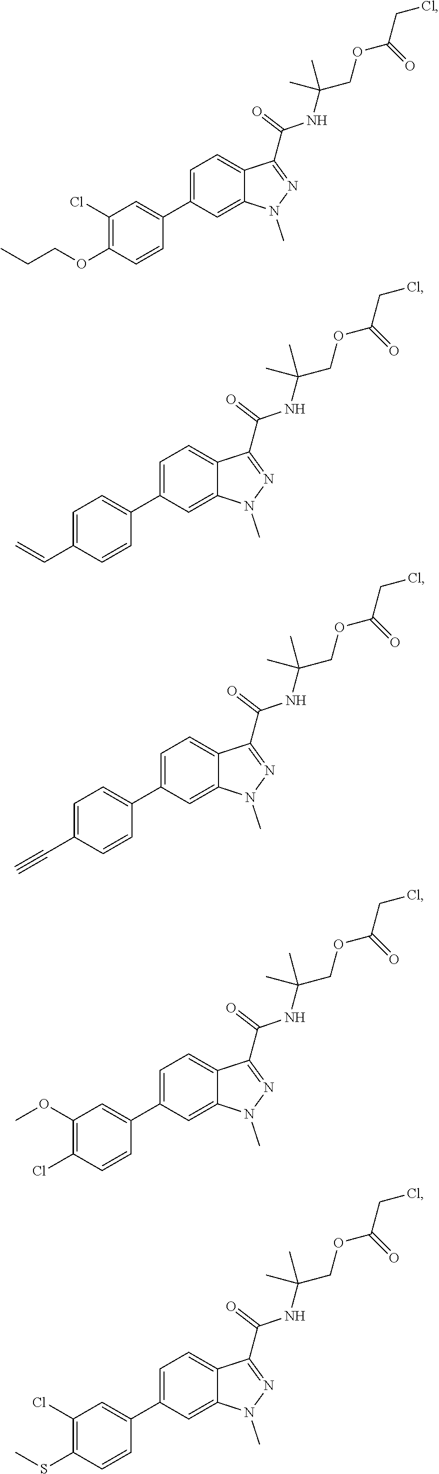 [SMILES: CCCOc1ccc(-c2ccc3c(C(=O)NC(C)(C)COC(=O)CCl)nn(C)c3c2)cc1Cl ; C=Cc1ccc(-c2ccc3c(C(=O)NC(C)(C)COC(=O)CCl)nn(C)c3c2)cc1 ; C#Cc1ccc(-c2ccc3c(C(=O)NC(C)(C)COC(=O)CCl)nn(C)c3c2)cc1 ; COc1cc(-c2ccc3c(C(=O)NC(C)(C)COC(=O)CCl)nn(C)c3c2)ccc1Cl ; CSc1ccc(-c2ccc3c(C(=O)NC(C)(C)COC(=O)CCl)nn(C)c3c2)cc1Cl] 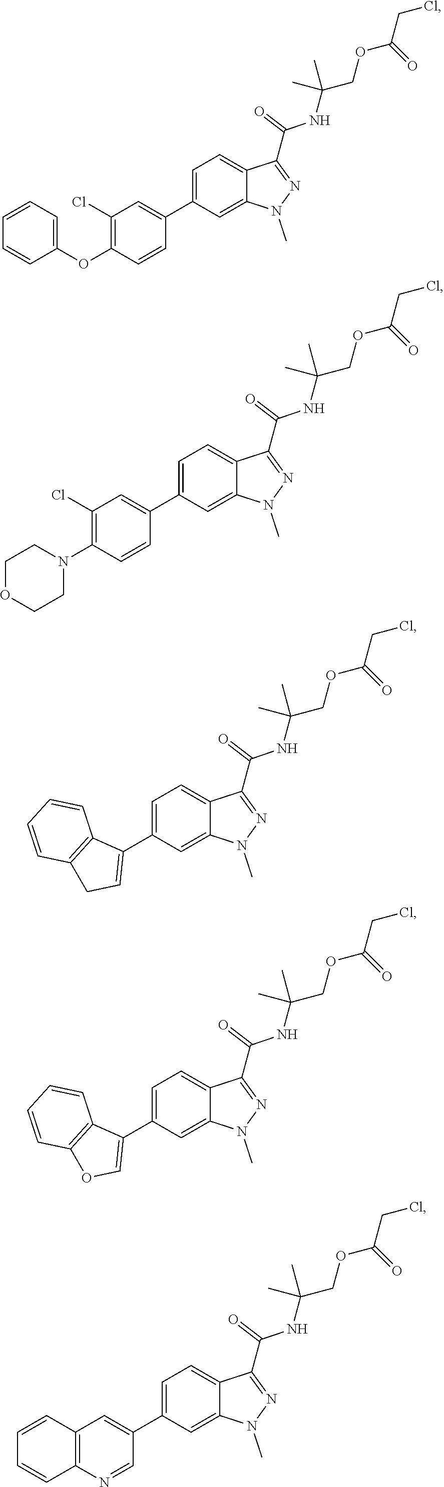 [SMILES: Cn1nc(C(=O)NC(C)(C)COC(=O)CCl)c2ccc(-c3ccc(Oc4ccccc4)c(Cl)c3)cc21 ; Cn1nc(C(=O)NC(C)(C)COC(=O)CCl)c2ccc(-c3ccc(N4CCOCC4)c(Cl)c3)cc21 ; Cn1nc(C(=O)NC(C)(C)COC(=O)CCl)c2ccc(C3=CCc4ccccc43)cc21 ; Cn1nc(C(=O)NC(C)(C)COC(=O)CCl)c2ccc(-c3coc4ccccc34)cc21 ; Cn1nc(C(=O)NC(C)(C)COC(=O)CCl)c2ccc(-c3cnc4ccccc4c3)cc21] 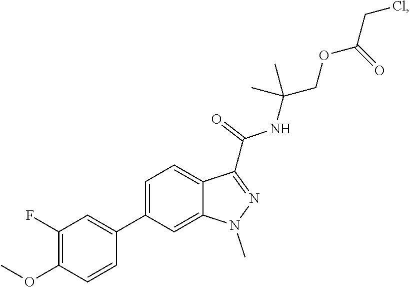 [SMILES: COc1ccc(-c2ccc3c(C(=O)NC(C)(C)COC(=O)CCl)nn(C)c3c2)cc1F] 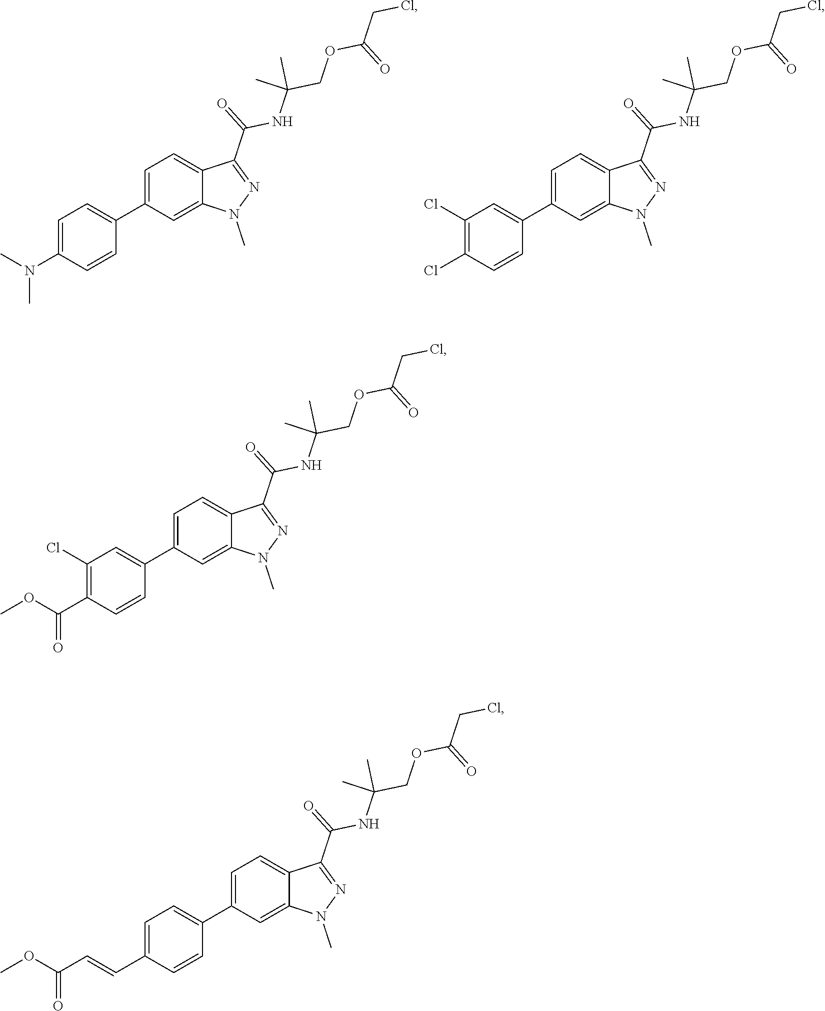 [SMILES: CN(C)c1ccc(-c2ccc3c(C(=O)NC(C)(C)COC(=O)CCl)nn(C)c3c2)cc1 ; Cn1nc(C(=O)NC(C)(C)COC(=O)CCl)c2ccc(-c3ccc(Cl)c(Cl)c3)cc21 ; COC(=O)c1ccc(-c2ccc3c(C(=O)NC(C)(C)COC(=O)CCl)nn(C)c3c2)cc1Cl ; COC(=O)/C=C/c1ccc(-c2ccc3c(C(=O)NC(C)(C)COC(=O)CCl)nn(C)c3c2)cc1] 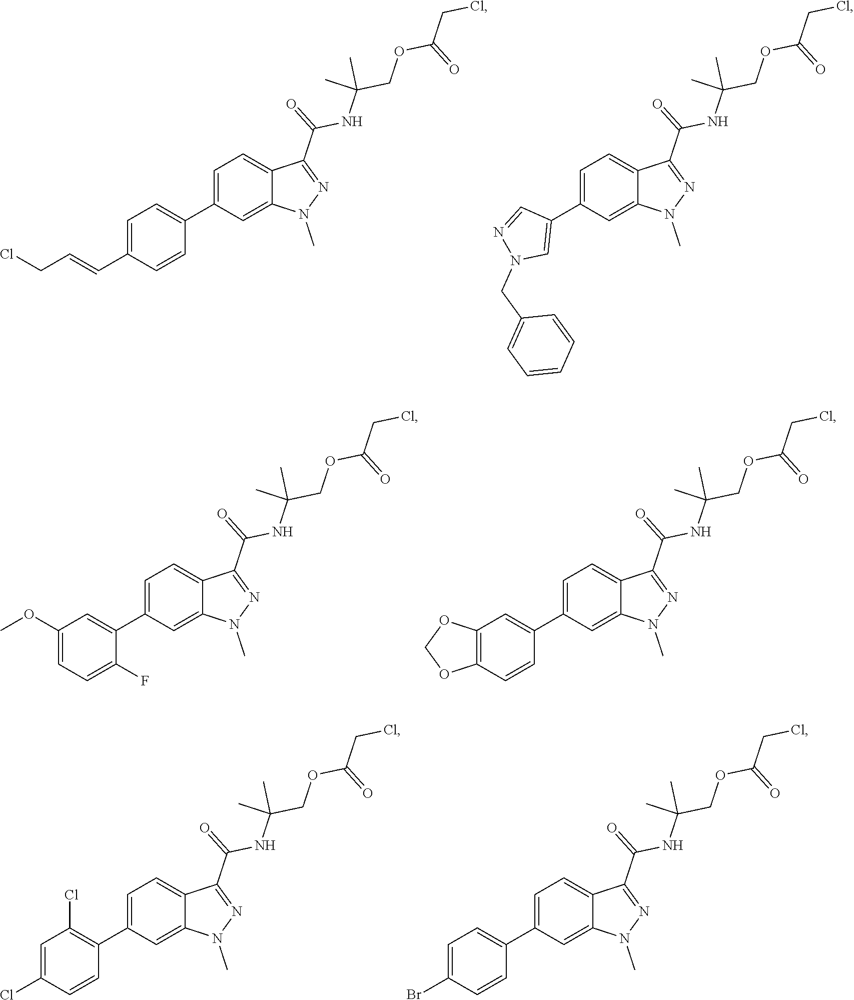 [SMILES: Cn1nc(C(=O)NC(C)(C)COC(=O)CCl)c2ccc(-c3ccc(/C=C/CCl)cc3)cc21 ; Cn1nc(C(=O)NC(C)(C)COC(=O)CCl)c2ccc(-c3cnn(Cc4ccccc4)c3)cc21 ; COc1ccc(F)c(-c2ccc3c(C(=O)NC(C)(C)COC(=O)CCl)nn(C)c3c2)c1 ; Cn1nc(C(=O)NC(C)(C)COC(=O)CCl)c2ccc(-c3ccc4c(c3)OCO4)cc21 ; Cn1nc(C(=O)NC(C)(C)COC(=O)CCl)c2ccc(-c3ccc(Cl)cc3Cl)cc21 ; Cn1nc(C(=O)NC(C)(C)COC(=O)CCl)c2ccc(-c3ccc(Br)cc3)cc21] 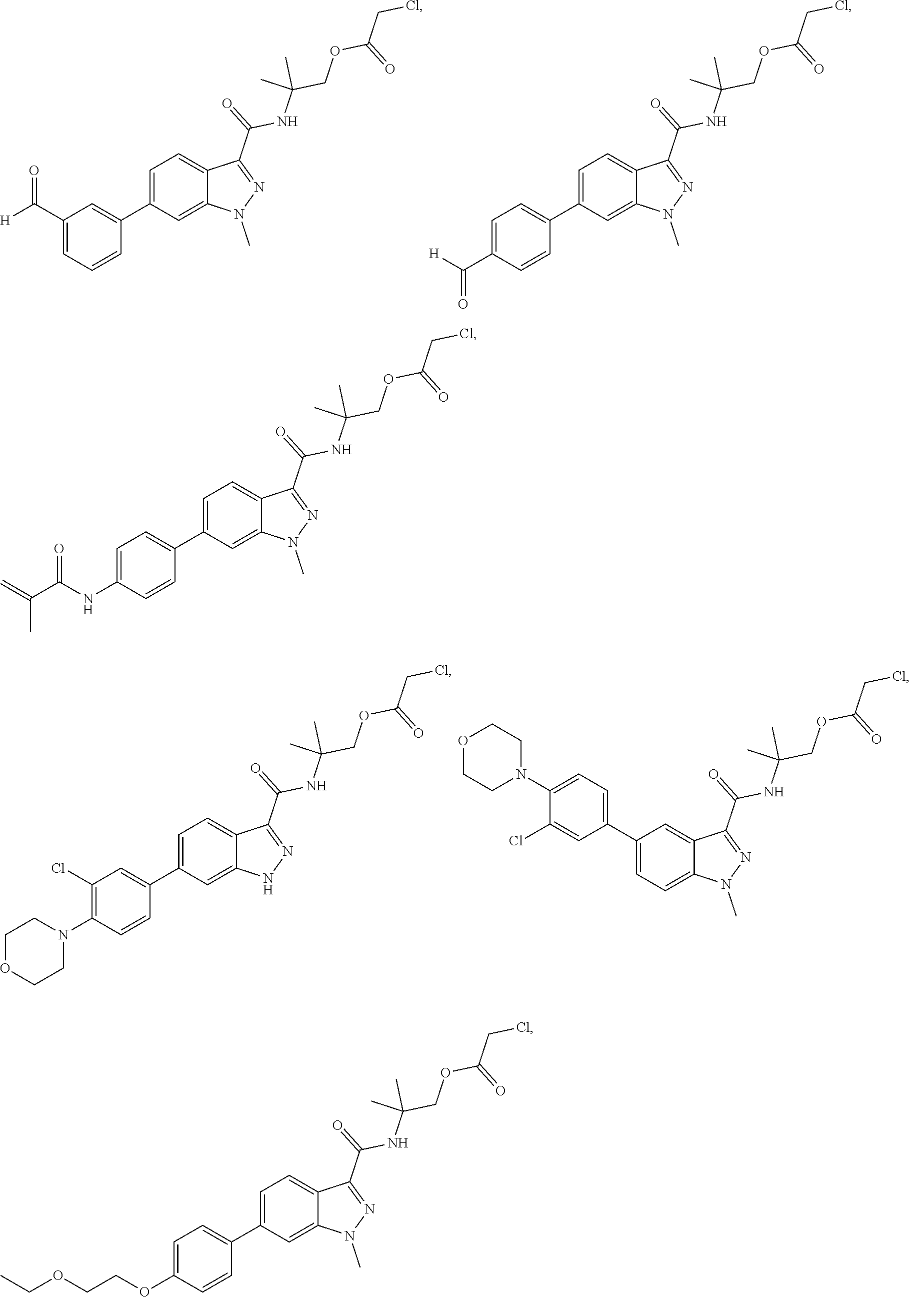 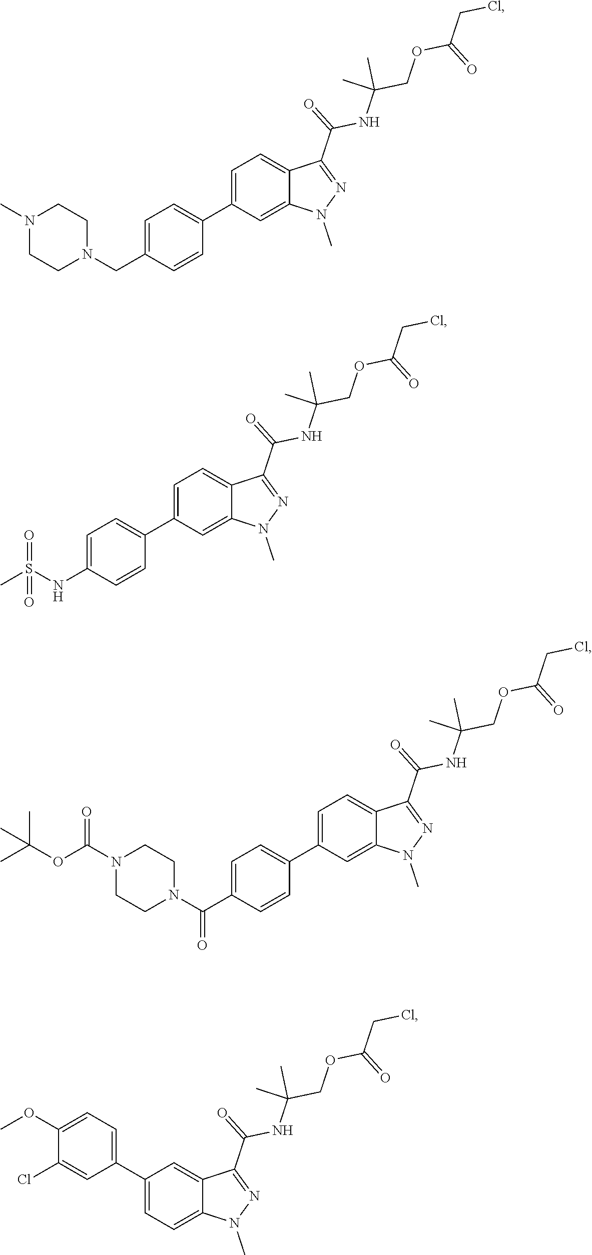 [SMILES: CN1CCN(Cc2ccc(-c3ccc4c(C(=O)NC(C)(C)COC(=O)CCl)nn(C)c4c3)cc2)CC1 ; Cn1nc(C(=O)NC(C)(C)COC(=O)CCl)c2ccc(-c3ccc(NS(C)(=O)=O)cc3)cc21 ; Cn1nc(C(=O)NC(C)(C)COC(=O)CCl)c2ccc(-c3ccc(C(=O)N4CCN(C(=O)OC(C)(C)C)CC4)cc3)cc21 ; COc1ccc(-c2ccc3c(c2)c(C(=O)NC(C)(C)COC(=O)CCl)nn3C)cc1Cl] 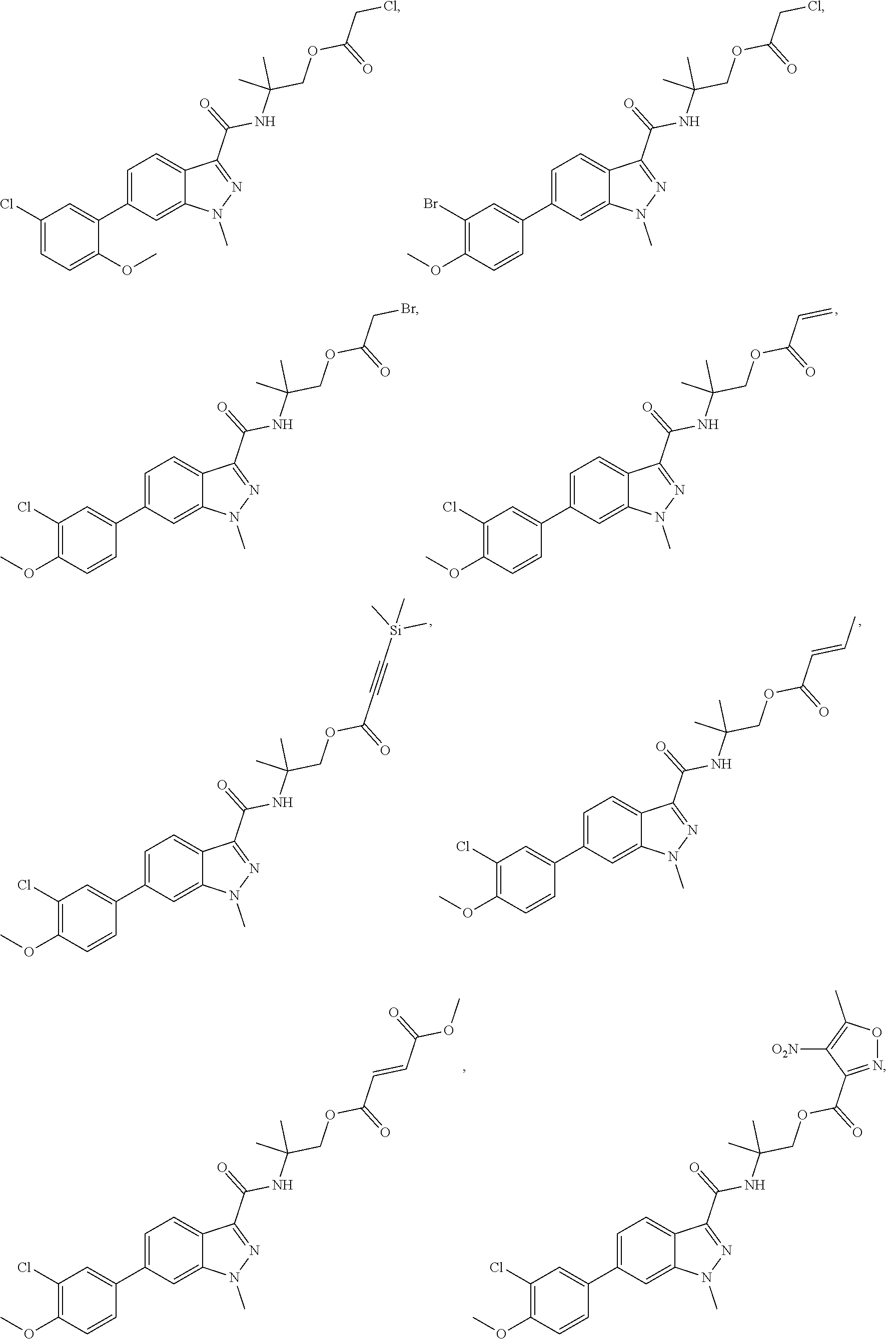 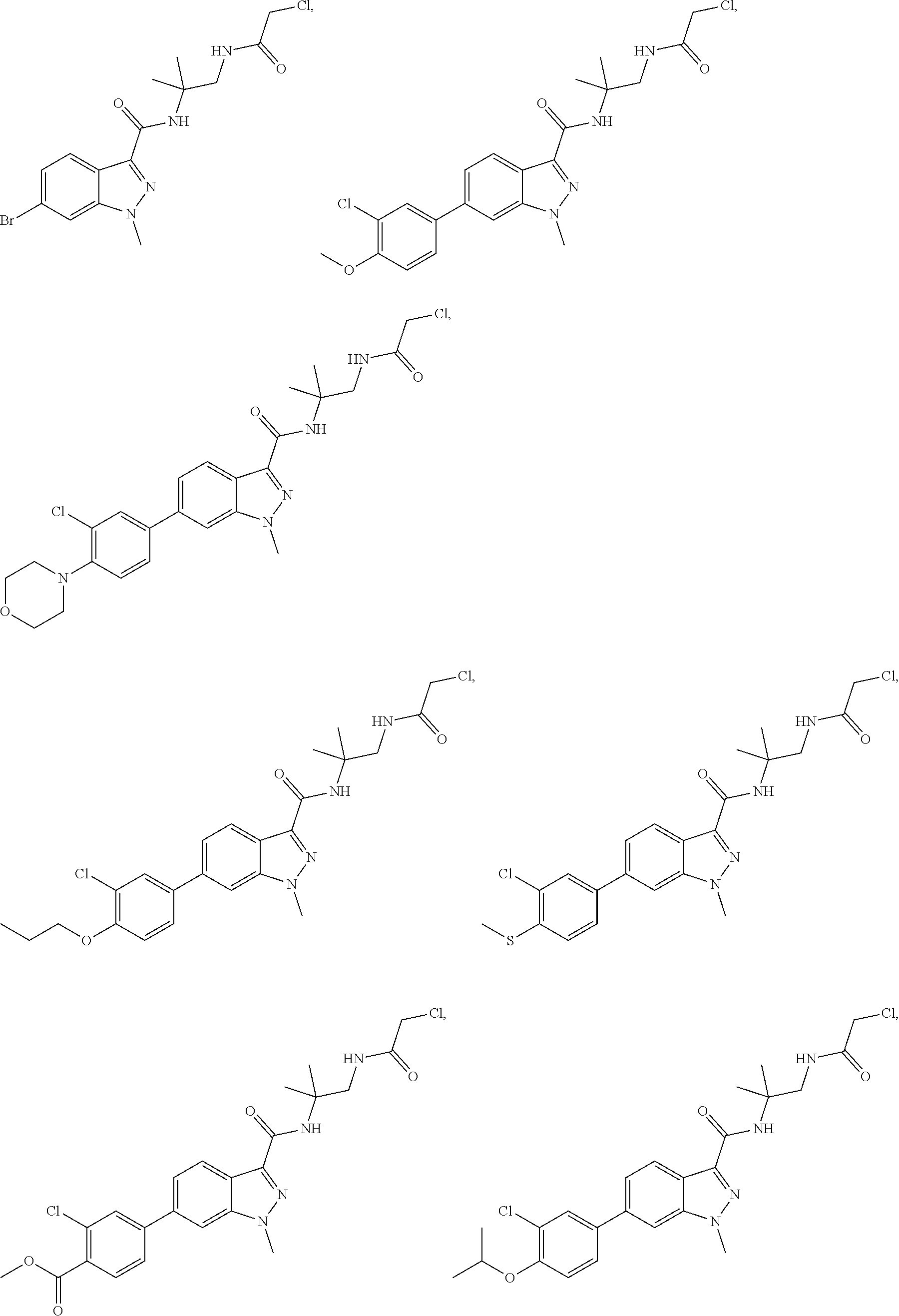 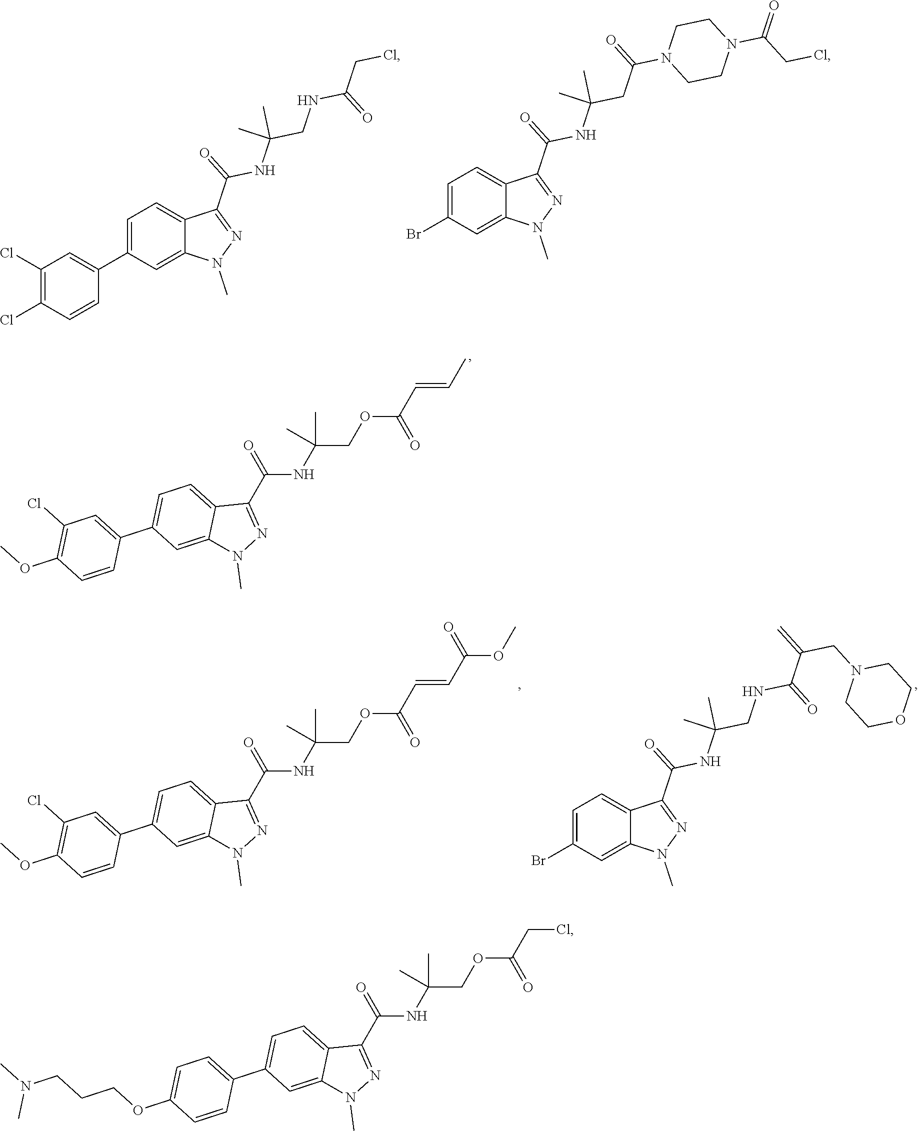 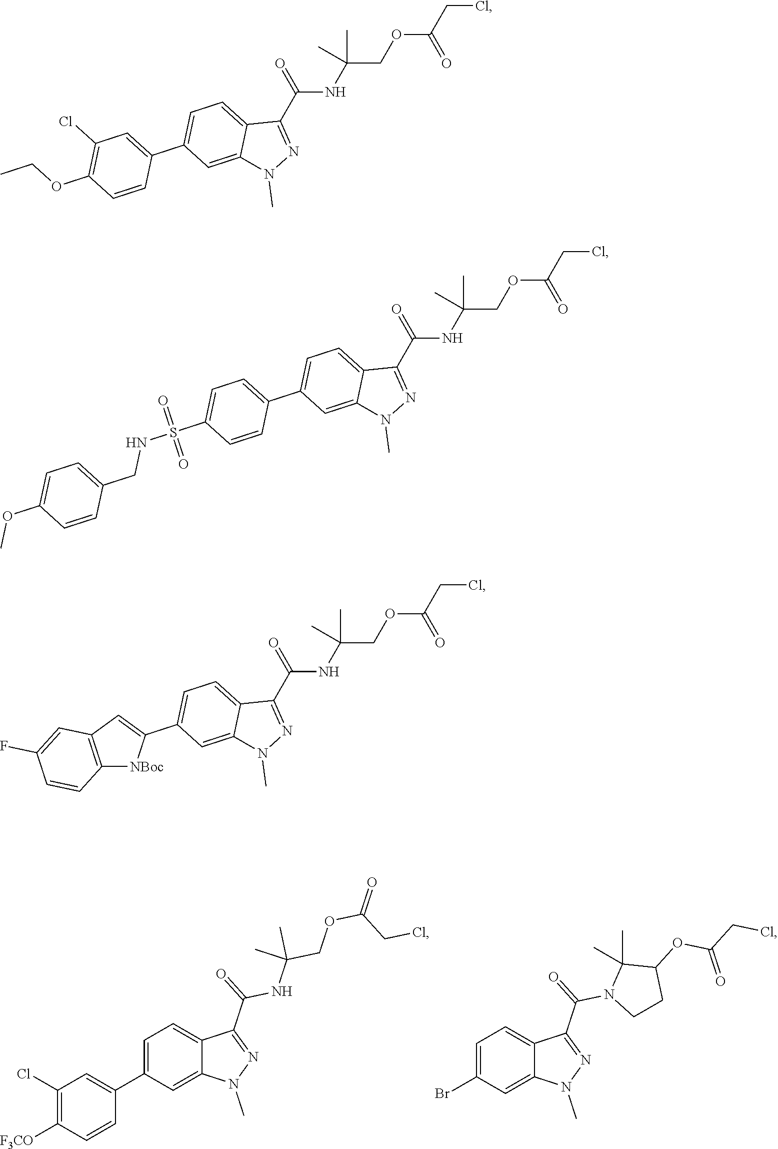 [SMILES: CCOc1ccc(-c2ccc3c(C(=O)NC(C)(C)COC(=O)CCl)nn(C)c3c2)cc1Cl ; COc1ccc(CNS(=O)(=O)c2ccc(-c3ccc4c(C(=O)NC(C)(C)COC(=O)CCl)nn(C)c4c3)cc2)cc1 ; Cn1nc(C(=O)NC(C)(C)COC(=O)CCl)c2ccc(-c3cc4cc(F)ccc4n3C(=O)OC(C)(C)C)cc21 ; Cn1nc(C(=O)NC(C)(C)COC(=O)CCl)c2ccc(-c3ccc(OC(F)(F)F)c(Cl)c3)cc21 ; Cn1nc(C(=O)N2CCC(OC(=O)CCl)C2(C)C)c2ccc(Br)cc21] 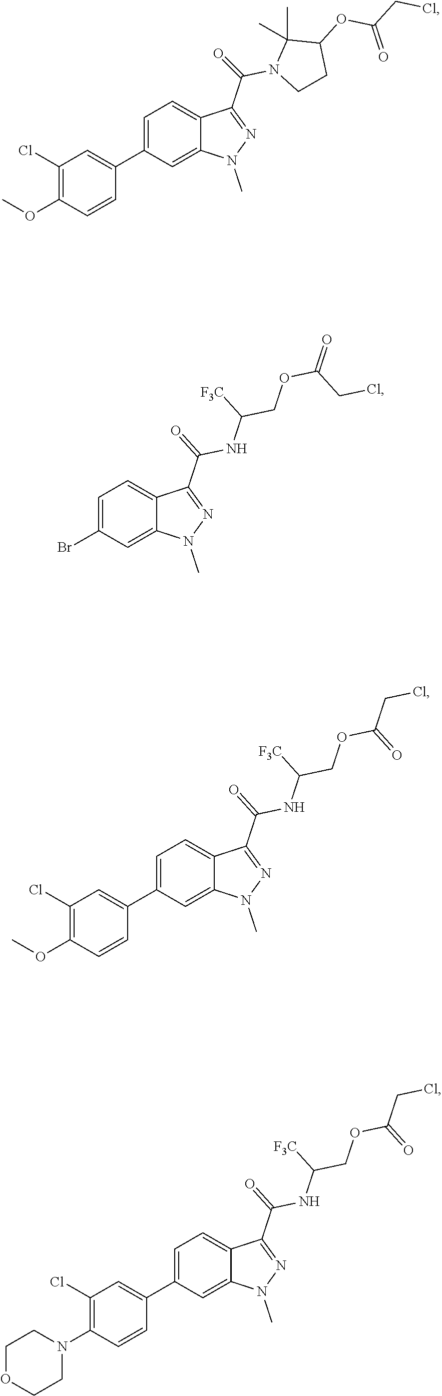 [SMILES: COc1ccc(-c2ccc3c(C(=O)N4CCC(OC(=O)CCl)C4(C)C)nn(C)c3c2)cc1Cl ; Cn1nc(C(=O)NC(COC(=O)CCl)C(F)(F)F)c2ccc(Br)cc21 ; COc1ccc(-c2ccc3c(C(=O)NC(COC(=O)CCl)C(F)(F)F)nn(C)c3c2)cc1Cl ; Cn1nc(C(=O)NC(COC(=O)CCl)C(F)(F)F)c2ccc(-c3ccc(N4CCOCC4)c(Cl)c3)cc21] 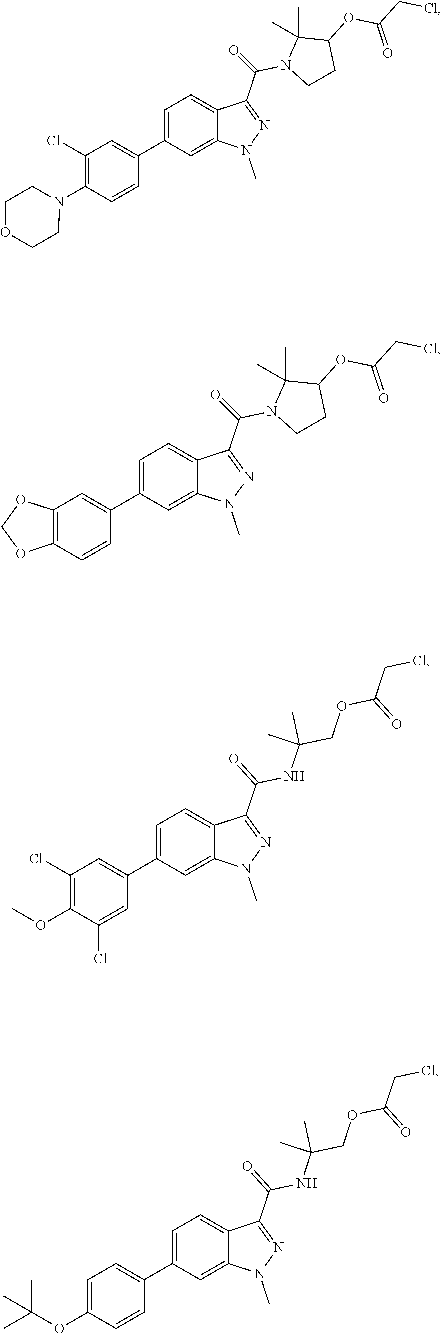 [SMILES: Cn1nc(C(=O)N2CCC(OC(=O)CCl)C2(C)C)c2ccc(-c3ccc(N4CCOCC4)c(Cl)c3)cc21 ; Cn1nc(C(=O)N2CCC(OC(=O)CCl)C2(C)C)c2ccc(-c3ccc4c(c3)OCO4)cc21 ; COc1c(Cl)cc(-c2ccc3c(C(=O)NC(C)(C)COC(=O)CCl)nn(C)c3c2)cc1Cl ; Cn1nc(C(=O)NC(C)(C)COC(=O)CCl)c2ccc(-c3ccc(OC(C)(C)C)cc3)cc21] 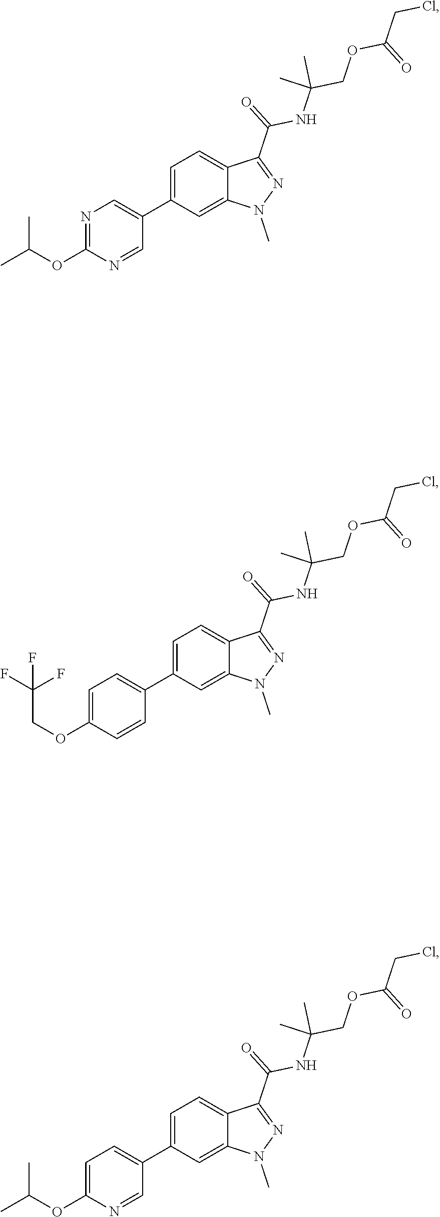 [SMILES: CC(C)Oc1ncc(-c2ccc3c(C(=O)NC(C)(C)COC(=O)CCl)nn(C)c3c2)cn1 ; Cn1nc(C(=O)NC(C)(C)COC(=O)CCl)c2ccc(-c3ccc(OCC(F)(F)F)cc3)cc21 ; CC(C)Oc1ccc(-c2ccc3c(C(=O)NC(C)(C)COC(=O)CCl)nn(C)c3c2)cn1] 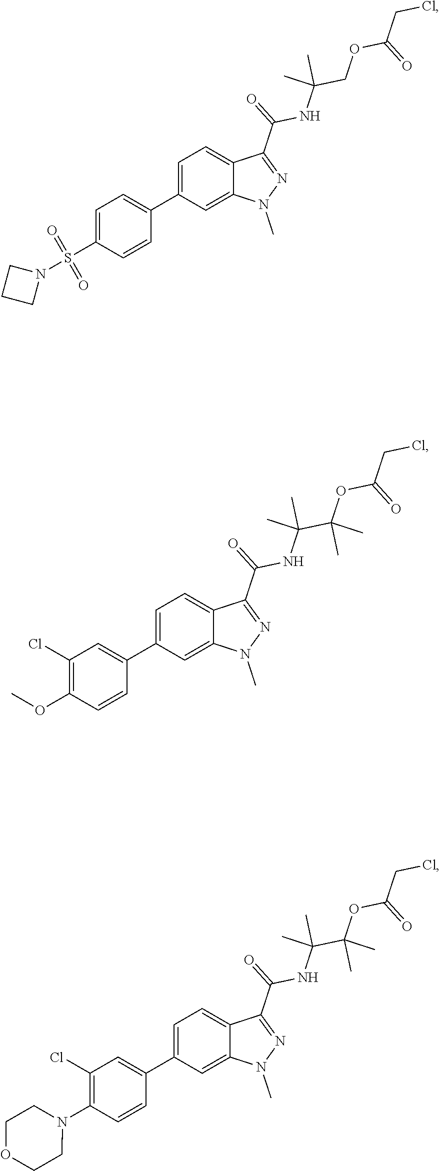 [SMILES: Cn1nc(C(=O)NC(C)(C)COC(=O)CCl)c2ccc(-c3ccc(S(=O)(=O)N4CCC4)cc3)cc21 ; COc1ccc(-c2ccc3c(C(=O)NC(C)(C)C(C)(C)OC(=O)CCl)nn(C)c3c2)cc1Cl ; Cn1nc(C(=O)NC(C)(C)C(C)(C)OC(=O)CCl)c2ccc(-c3ccc(N4CCOCC4)c(Cl)c3)cc21] 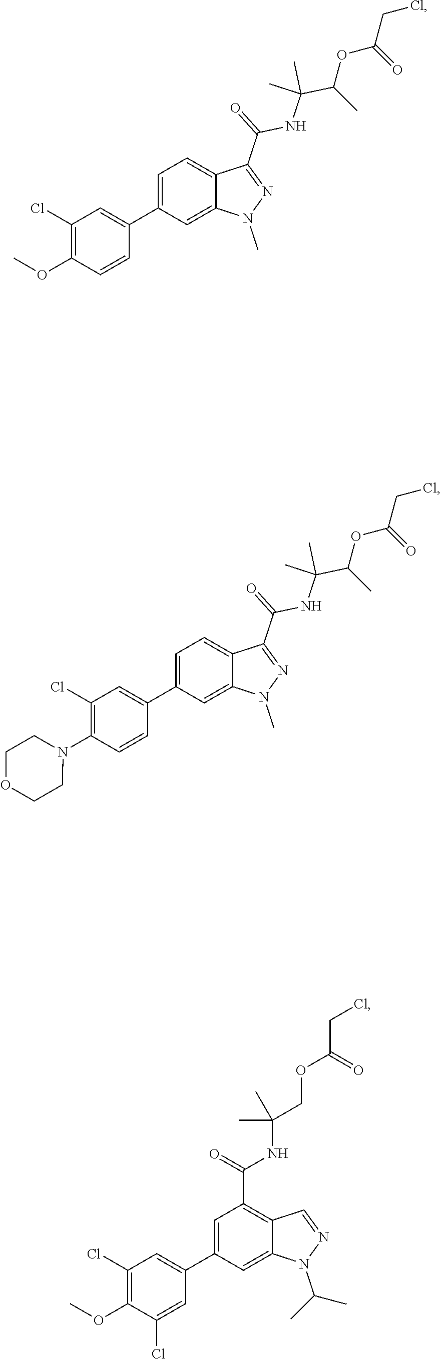 [SMILES: COc1ccc(-c2ccc3c(C(=O)NC(C)(C)C(C)OC(=O)CCl)nn(C)c3c2)cc1Cl ; CC(OC(=O)CCl)C(C)(C)NC(=O)c1nn(C)c2cc(-c3ccc(N4CCOCC4)c(Cl)c3)ccc12 ; COc1c(Cl)cc(-c2cc(C(=O)NC(C)(C)COC(=O)CCl)c3cnn(C(C)C)c3c2)cc1Cl] 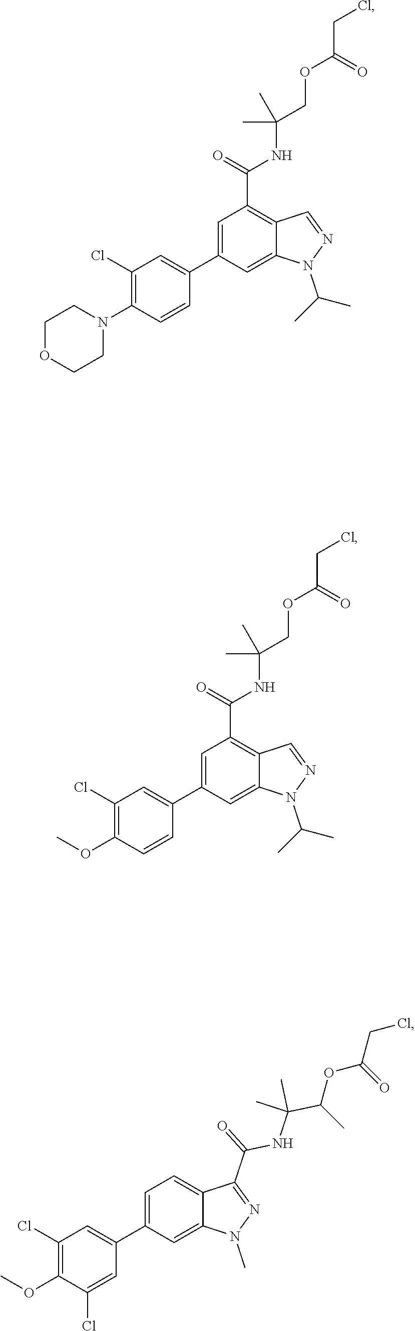 [SMILES: CC(C)n1ncc2c(C(=O)NC(C)(C)COC(=O)CCl)cc(-c3ccc(N4CCOCC4)c(Cl)c3)cc21 ; COc1ccc(-c2cc(C(=O)NC(C)(C)COC(=O)CCl)c3cnn(C(C)C)c3c2)cc1Cl ; COc1c(Cl)cc(-c2ccc3c(C(=O)NC(C)(C)C(C)OC(=O)CCl)nn(C)c3c2)cc1Cl] 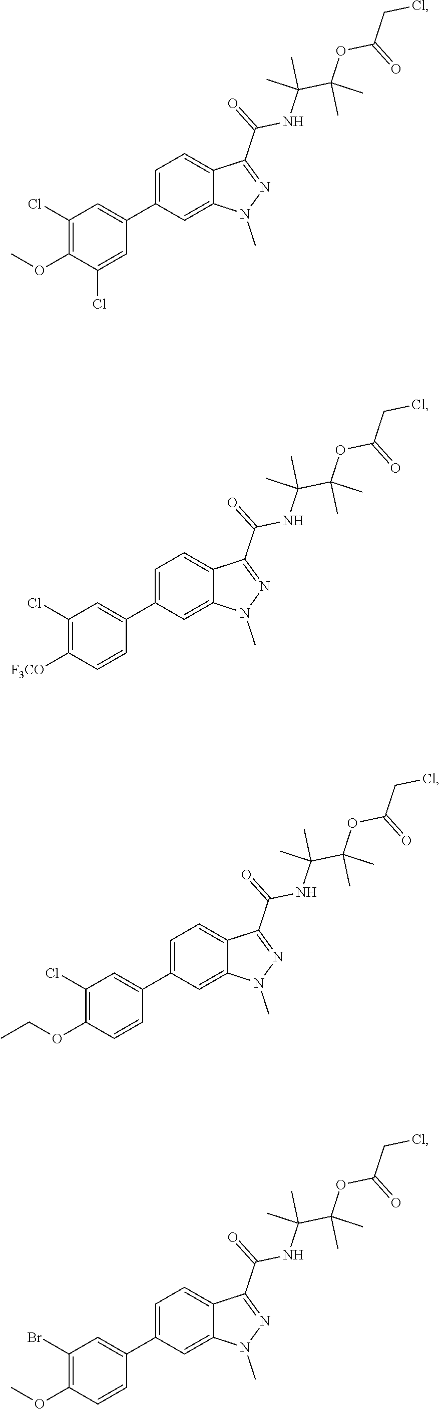 [SMILES: COc1c(Cl)cc(-c2ccc3c(C(=O)NC(C)(C)C(C)(C)OC(=O)CCl)nn(C)c3c2)cc1Cl ; Cn1nc(C(=O)NC(C)(C)C(C)(C)OC(=O)CCl)c2ccc(-c3ccc(OC(F)(F)F)c(Cl)c3)cc21 ; CCOc1ccc(-c2ccc3c(C(=O)NC(C)(C)C(C)(C)OC(=O)CCl)nn(C)c3c2)cc1Cl ; COc1ccc(-c2ccc3c(C(=O)NC(C)(C)C(C)(C)OC(=O)CCl)nn(C)c3c2)cc1Br] 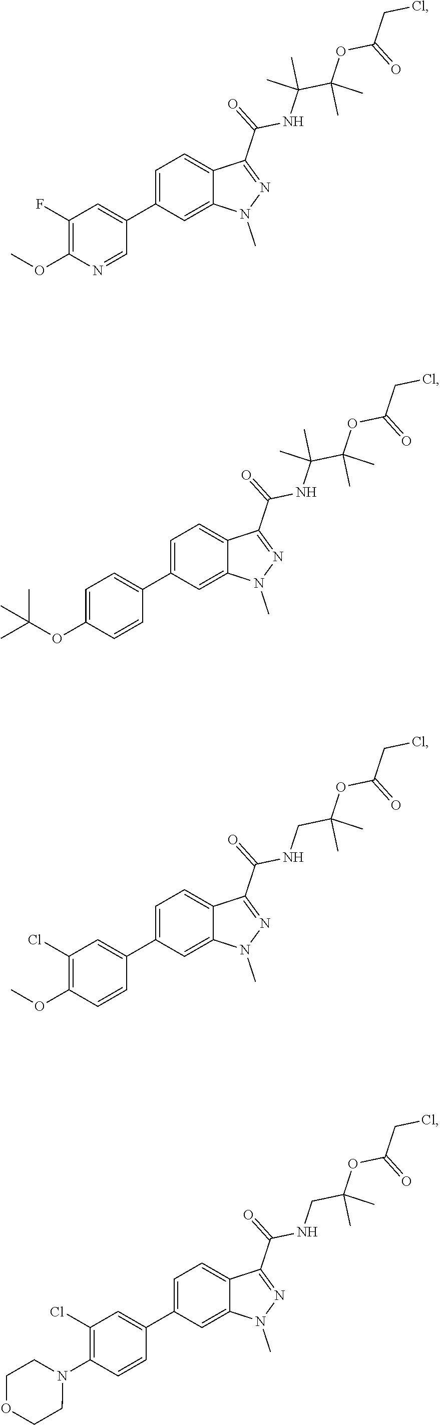 [SMILES: COc1ncc(-c2ccc3c(C(=O)NC(C)(C)C(C)(C)OC(=O)CCl)nn(C)c3c2)cc1F ; Cn1nc(C(=O)NC(C)(C)C(C)(C)OC(=O)CCl)c2ccc(-c3ccc(OC(C)(C)C)cc3)cc21 ; COc1ccc(-c2ccc3c(C(=O)NCC(C)(C)OC(=O)CCl)nn(C)c3c2)cc1Cl ; Cn1nc(C(=O)NCC(C)(C)OC(=O)CCl)c2ccc(-c3ccc(N4CCOCC4)c(Cl)c3)cc21] 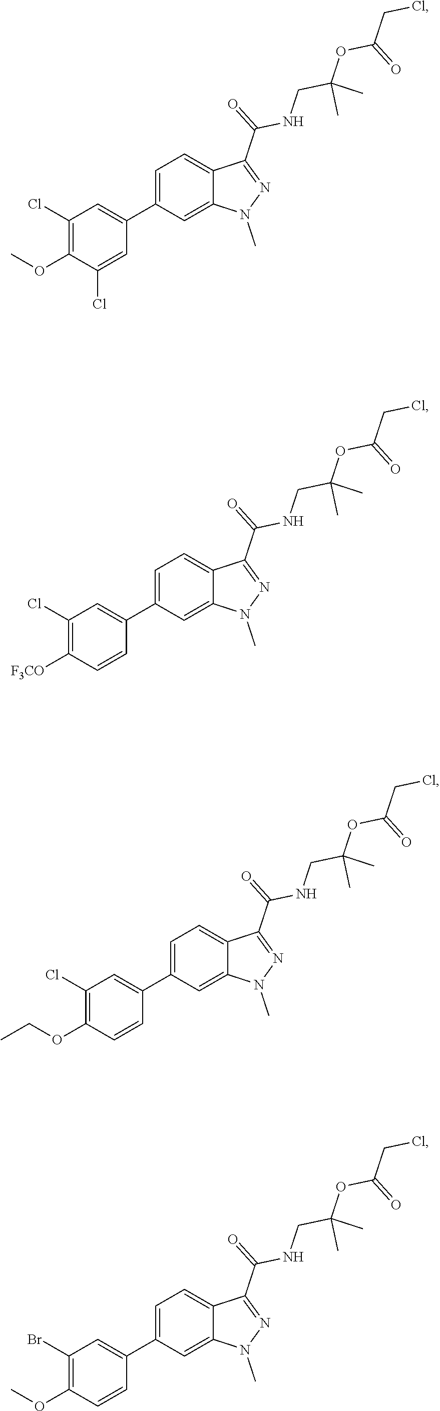 [SMILES: COc1c(Cl)cc(-c2ccc3c(C(=O)NCC(C)(C)OC(=O)CCl)nn(C)c3c2)cc1Cl ; Cn1nc(C(=O)NCC(C)(C)OC(=O)CCl)c2ccc(-c3ccc(OC(F)(F)F)c(Cl)c3)cc21 ; CCOc1ccc(-c2ccc3c(C(=O)NCC(C)(C)OC(=O)CCl)nn(C)c3c2)cc1Cl ; COc1ccc(-c2ccc3c(C(=O)NCC(C)(C)OC(=O)CCl)nn(C)c3c2)cc1Br] 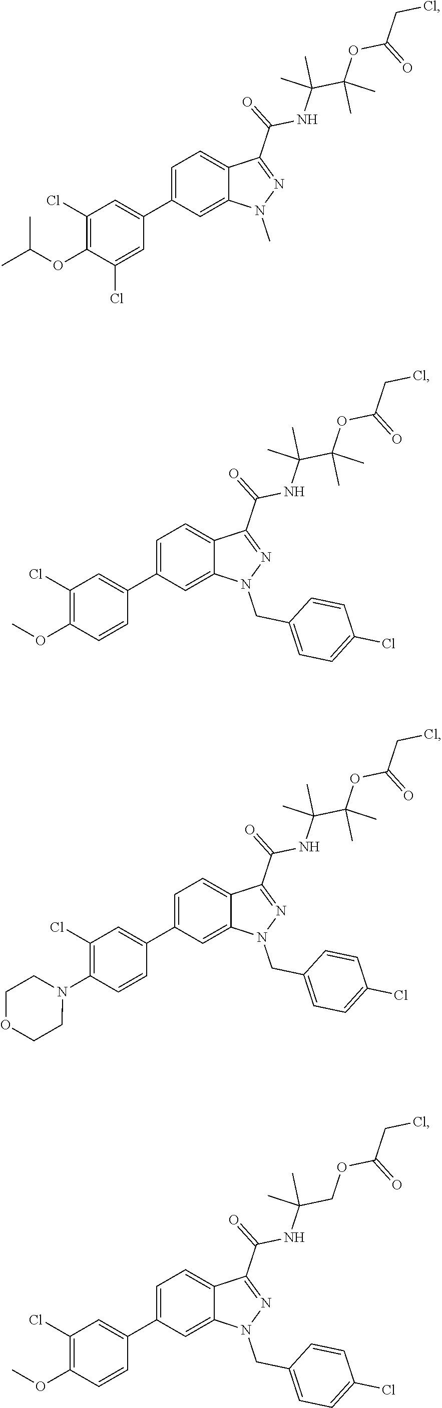 [SMILES: CC(C)Oc1c(Cl)cc(-c2ccc3c(C(=O)NC(C)(C)C(C)(C)OC(=O)CCl)nn(C)c3c2)cc1Cl ; COc1ccc(-c2ccc3c(C(=O)NC(C)(C)C(C)(C)OC(=O)CCl)nn(Cc4ccc(Cl)cc4)c3c2)cc1Cl ; CC(C)(NC(=O)c1nn(Cc2ccc(Cl)cc2)c2cc(-c3ccc(N4CCOCC4)c(Cl)c3)ccc12)C(C)(C)OC(=O)CCl ; COc1ccc(-c2ccc3c(C(=O)NC(C)(C)COC(=O)CCl)nn(Cc4ccc(Cl)cc4)c3c2)cc1Cl] 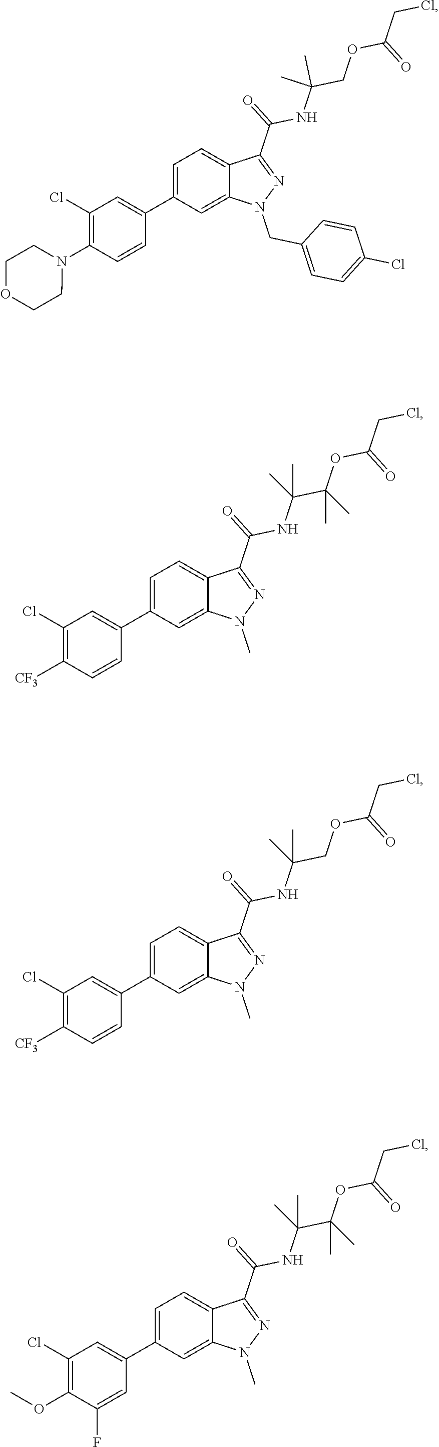 [SMILES: CC(C)(COC(=O)CCl)NC(=O)c1nn(Cc2ccc(Cl)cc2)c2cc(-c3ccc(N4CCOCC4)c(Cl)c3)ccc12 ; Cn1nc(C(=O)NC(C)(C)C(C)(C)OC(=O)CCl)c2ccc(-c3ccc(C(F)(F)F)c(Cl)c3)cc21 ; Cn1nc(C(=O)NC(C)(C)COC(=O)CCl)c2ccc(-c3ccc(C(F)(F)F)c(Cl)c3)cc21 ; COc1c(F)cc(-c2ccc3c(C(=O)NC(C)(C)C(C)(C)OC(=O)CCl)nn(C)c3c2)cc1Cl] 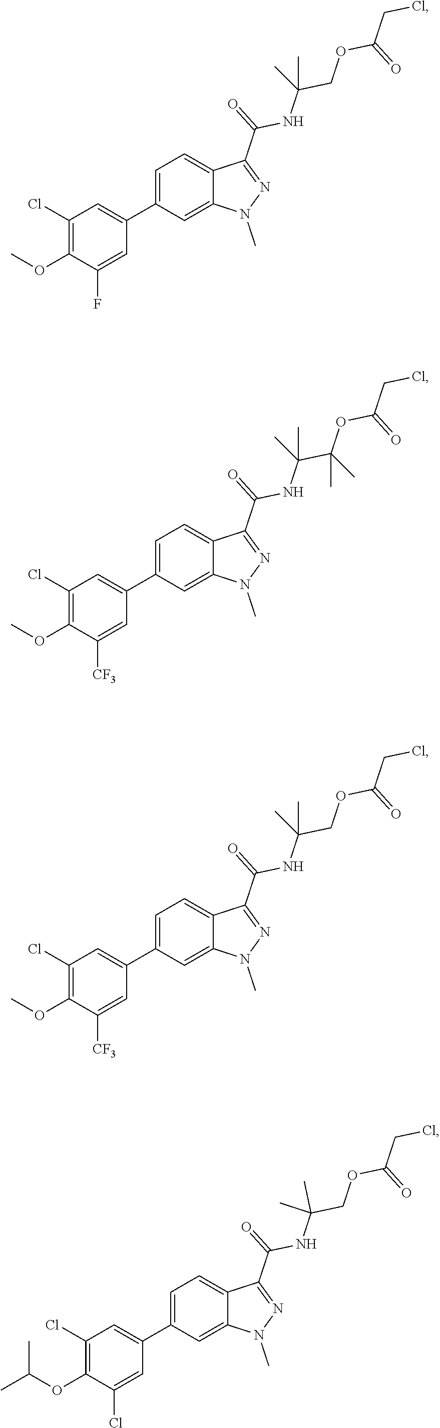 [SMILES: COc1c(F)cc(-c2ccc3c(C(=O)NC(C)(C)COC(=O)CCl)nn(C)c3c2)cc1Cl ; COc1c(Cl)cc(-c2ccc3c(C(=O)NC(C)(C)C(C)(C)OC(=O)CCl)nn(C)c3c2)cc1C(F)(F)F ; COc1c(Cl)cc(-c2ccc3c(C(=O)NC(C)(C)COC(=O)CCl)nn(C)c3c2)cc1C(F)(F)F ; CC(C)Oc1c(Cl)cc(-c2ccc3c(C(=O)NC(C)(C)COC(=O)CCl)nn(C)c3c2)cc1Cl] 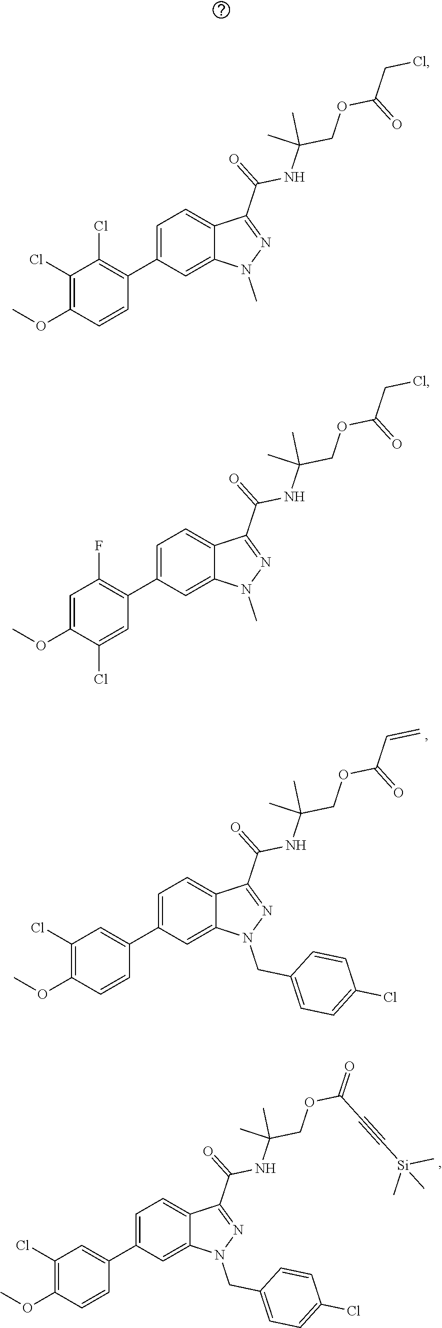 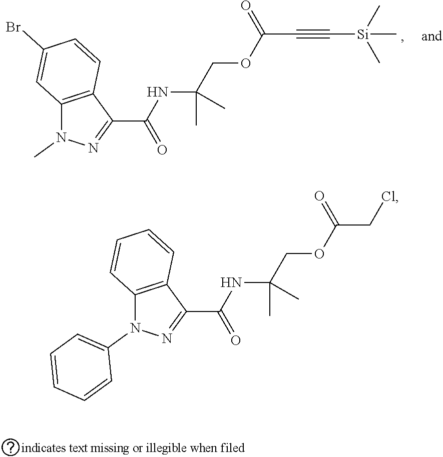 [SMILES: Cn1nc(C(=O)NC(C)(C)COC(=O)C#C[Si](C)(C)C)c2ccc(Br)cc21 ; CC(C)(COC(=O)CCl)NC(=O)c1nn(-c2ccccc2)c2ccccc12]
or an N-oxide, crystalline form, hydrate, or pharmaceutically acceptable salt thereof.

3. The compound according to claim 1 having a structure selected from the group consisting of: 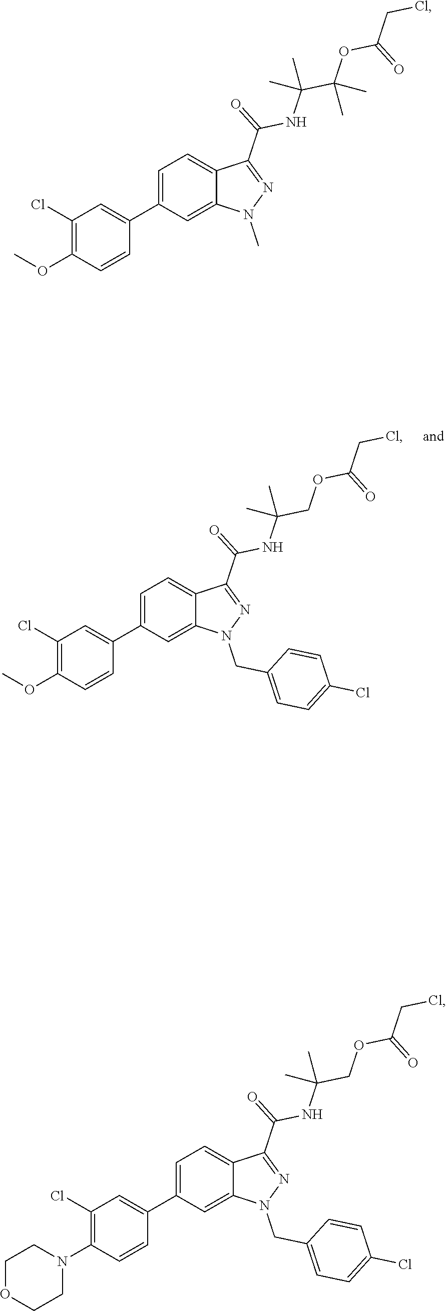 [SMILES: COc1ccc(-c2ccc3c(C(=O)NC(C)(C)C(C)(C)OC(=O)CCl)nn(C)c3c2)cc1Cl ; COc1ccc(-c2ccc3c(C(=O)NC(C)(C)COC(=O)CCl)nn(Cc4ccc(Cl)cc4)c3c2)cc1Cl ; CC(C)(COC(=O)CCl)NC(=O)c1nn(Cc2ccc(Cl)cc2)c2cc(-c3ccc(N4CCOCC4)c(Cl)c3)ccc12]
or an N-oxide, crystalline form, hydrate, or pharmaceutically acceptable salt thereof.

4. A compound according to formula (2): 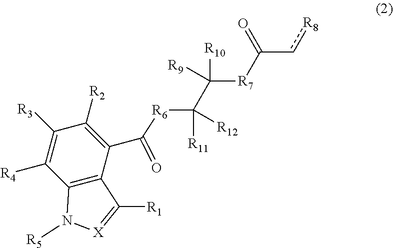
wherein:
a dashed line indicates the presence of an optional double bond;
X is selected from the group consisting of O, N, S, and C;
R₁, R₂, R₃, R₄ and R₅ are independently selected from the group consisting of H, D, —OH, halo, ether, ester, furanyl, indole, indazole, pyrrole, pyrazole, pyridine, pyrimidine, naphthalene, indene, dibenzofuran, benzodioxole, amide, —(O)C(R), —C(O)OR, —NO₂, alkyl, aryl, alkyl-aryl, alkyl-heteroaryl, alkenyl, alkenyl-aryl, alkenyl-heteroaryl, peptide, and polypeptide, wherein the ether, ester, furanyl, indole, indazole, pyrrole, pyrazole, pyridine, pyrimidine, naphthalene, indene, dibenzofuran, benzodioxole, amide, alkyl, aryl, alkyl-aryl, alkyl-heteroaryl, alkenyl, alkenyl-aryl, alkenyl-heteroaryl, peptide, and polypeptide may be optionally substituted with an atom or a group selected from the group consisting of O, N, S, epoxy, —OH, halo, —CN, —NO₂, C₁₋₄alkyl, CF₃, —CF₃O, carbonyl, alkyl-aryl, thiol, methylmethanesulfonamide, methylthiol, dioxane, tetrahydropyzan, morpholinyl, piperazine, sulfonyl, oxazole, oxadiazole, dizaole, triazole, amide, ether, ester and combinations thereof; or R₁ and R₂ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₂ and R₃ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₃ and R₄ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof,
R₆ and R₇ are independently selected from the group consisting of C, N, O and S; or R₆ and R₇ may together form a saturated or unsaturated ring structure that may be optionally substituted with an atom or a group from the group consisting of O, N, halo, C₁₋₄alkyl, CF₃, oxazole, oxadiazole, dizaole, triazole, amide, ether, ester and combinations thereof,
R₈ is selected from the group consisting of H, D, C, O, N, alkenyl, alkynyl, ester, oxazole, piperazine, morpholinyl and halo,
R₉, R₁₀, R₁₁ and R₁₂ are independently selected from the group consisting of no atom, H, D, —OH, halo, —CN, —NO₂, C₁₋₄alkyl, CF₃, —CF₃O, carbonyl, aryl, alkyl-aryl, thiol, ether, and ester,
wherein R is selected from the group consisting of H, D, O, N, halo, oxazole, oxadiazole, dizaole, triazole, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of O, N, S, halo, C₁₋₄alkyl, CF₃, —CN and combinations thereof,
or an N-oxide, crystalline form, hydrate, or pharmaceutically acceptable salt thereof.

5. A compound according to formula (3): 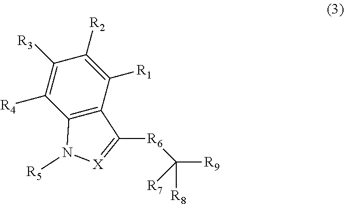
wherein:
X is selected from the group consisting of O, N, S, and C;
R₁, R₂, R₃, R₄, R₅ and R₉ are independently selected from the group consisting of H, D, —OH, halo, ether, ester, furanyl, indole, indazole, pyrrole, pyrazole, pyridine, pyrimidine, naphthalene, indene, dibenzofuran, benzodioxole, amide, —(O)C(R), —C(O)OR, —NO₂, alkyl, aryl, alkyl-aryl, alkyl-heteroaryl, alkenyl, alkenyl-aryl, alkenyl-heteroaryl, peptide, and polypeptide, wherein the ether, ester, furanyl, indole, indazole, pyrrole, pyrazole, pyridine, pyrimidine, naphthalene, indene, dibenzofuran, benzodioxole, amide, alkyl, aryl, alkyl-aryl, alkyl-heteroaryl, alkenyl, alkenyl-aryl, alkenyl-heteroaryl, peptide, and polypeptide may be optionally substituted with an atom or a group selected from the group consisting of O, N, S, epoxy, —OH, halo, —CN, —NO₂, C₁₋₄alkyl, CF₃, —CF₃O, carbonyl, alkyl-aryl, thiol, methylmethanesulfonamide, methylthiol, dioxane, tetrahydropyzan, morpholinyl, piperazine, sulfonyl, oxazole, oxadiazole, dizaole, triazole, amide, ether, ester and combinations thereof; or R₁ and R₂ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₂ and R₃ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₃ and R₄ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof,
R₆ is selected from the group consisting of amide, oxazole, oxadiazole, diazole, and triazole,
R₇ and R₈ are independently selected from the group consisting of no atom, H, D, —OH, halo, —CN, —NO₂, C₁₋₄alkyl, CF₃, —CF₃O, carbonyl, aryl, alkyl-aryl, thiol, ether, and ester,
wherein R is selected from the group consisting of H, D, O, N, halo, oxazole, oxadiazole, dizaole, triazole, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of O, N, S, halo, C₁₋₄alkyl, CF₃, —CN and combinations thereof,
or an N-oxide, crystalline form, hydrate, or pharmaceutically acceptable salt thereof.

6. The compound according to claim 5 having a structure selected from the group consisting of: 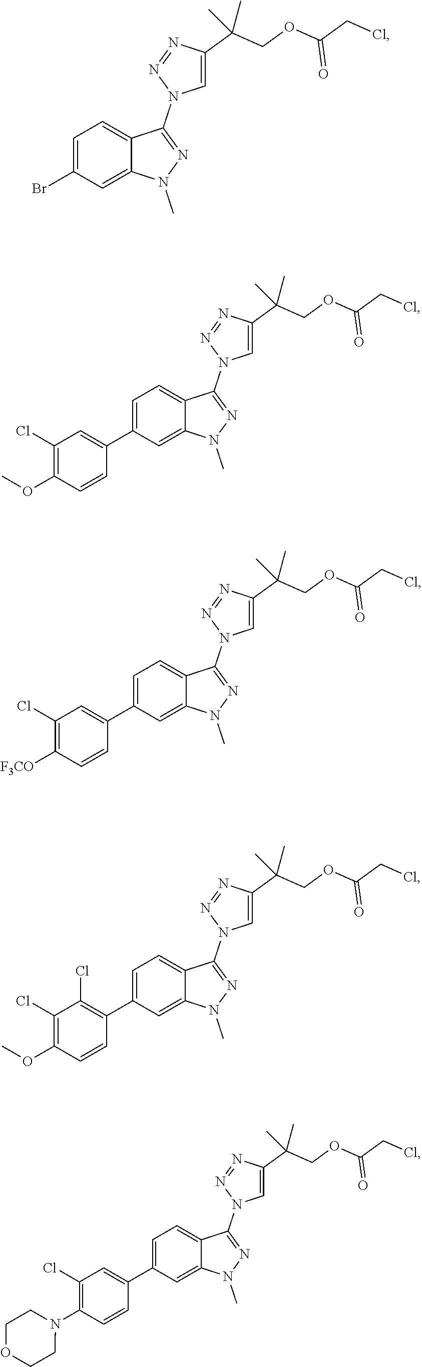 [SMILES: Cn1nc(-n2cc(C(C)(C)COC(=O)CCl)nn2)c2ccc(Br)cc21 ; COc1ccc(-c2ccc3c(-n4cc(C(C)(C)COC(=O)CCl)nn4)nn(C)c3c2)cc1Cl ; Cn1nc(-n2cc(C(C)(C)COC(=O)CCl)nn2)c2ccc(-c3ccc(OC(F)(F)F)c(Cl)c3)cc21 ; COc1ccc(-c2ccc3c(-n4cc(C(C)(C)COC(=O)CCl)nn4)nn(C)c3c2)c(Cl)c1Cl ; Cn1nc(-n2cc(C(C)(C)COC(=O)CCl)nn2)c2ccc(-c3ccc(N4CCOCC4)c(Cl)c3)cc21] 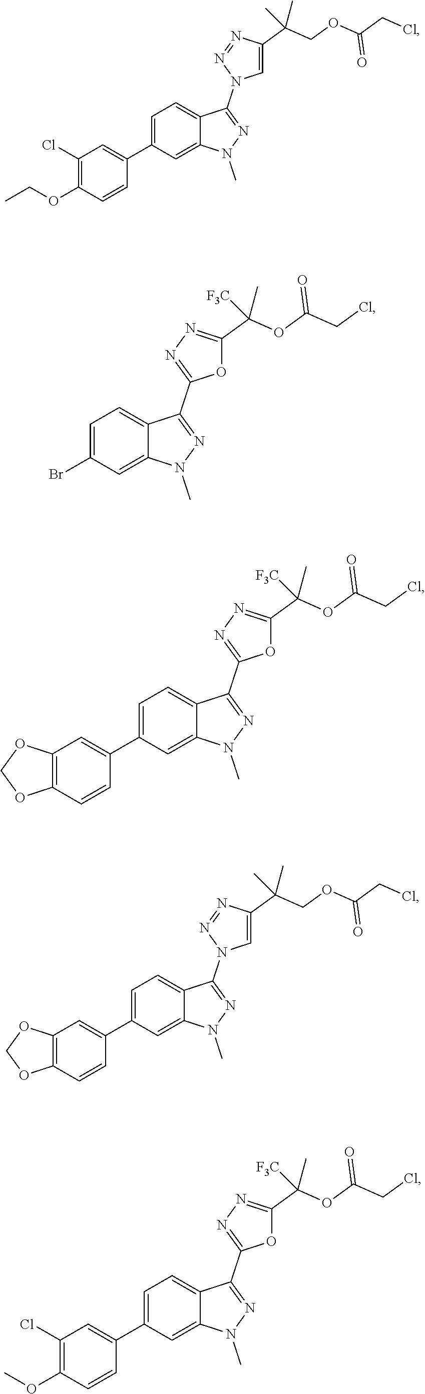 [SMILES: CCOc1ccc(-c2ccc3c(-n4cc(C(C)(C)COC(=O)CCl)nn4)nn(C)c3c2)cc1Cl ; Cn1nc(-c2nnc(C(C)(OC(=O)CCl)C(F)(F)F)o2)c2ccc(Br)cc21 ; Cn1nc(-c2nnc(C(C)(OC(=O)CCl)C(F)(F)F)o2)c2ccc(-c3ccc4c(c3)OCO4)cc21 ; Cn1nc(-n2cc(C(C)(C)COC(=O)CCl)nn2)c2ccc(-c3ccc4c(c3)OCO4)cc21 ; COc1ccc(-c2ccc3c(-c4nnc(C(C)(OC(=O)CCl)C(F)(F)F)o4)nn(C)c3c2)cc1Cl] 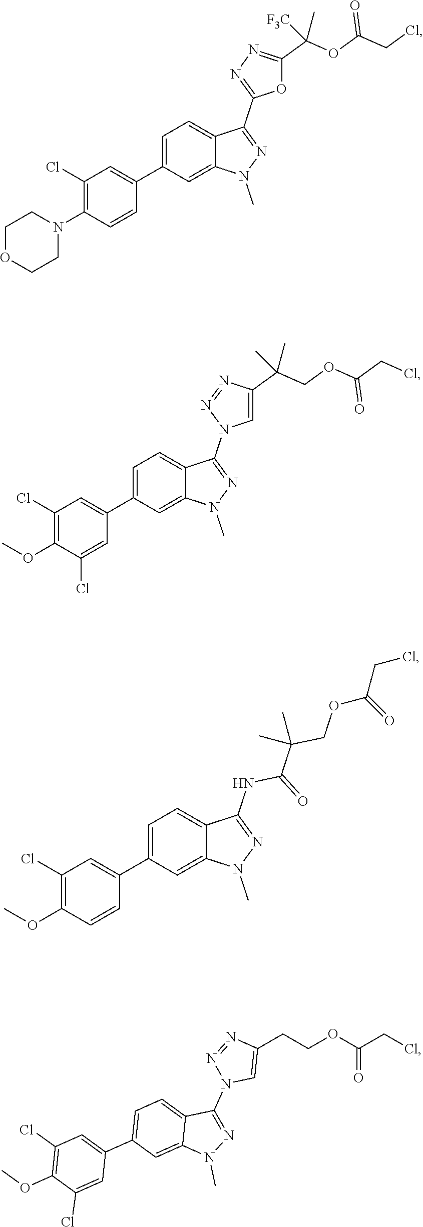 [SMILES: Cn1nc(-c2nnc(C(C)(OC(=O)CCl)C(F)(F)F)o2)c2ccc(-c3ccc(N4CCOCC4)c(Cl)c3)cc21 ; COc1c(Cl)cc(-c2ccc3c(-n4cc(C(C)(C)COC(=O)CCl)nn4)nn(C)c3c2)cc1Cl ; COc1ccc(-c2ccc3c(NC(=O)C(C)(C)COC(=O)CCl)nn(C)c3c2)cc1Cl ; COc1c(Cl)cc(-c2ccc3c(-n4cc(CCOC(=O)CCl)nn4)nn(C)c3c2)cc1Cl] 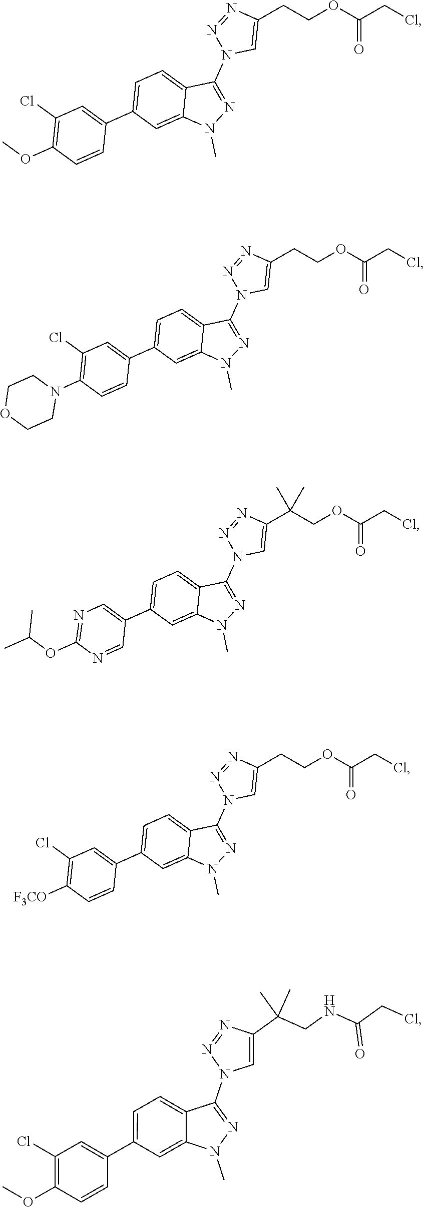 [SMILES: COc1ccc(-c2ccc3c(-n4cc(CCOC(=O)CCl)nn4)nn(C)c3c2)cc1Cl ; Cn1nc(-n2cc(CCOC(=O)CCl)nn2)c2ccc(-c3ccc(N4CCOCC4)c(Cl)c3)cc21 ; CC(C)Oc1ncc(-c2ccc3c(-n4cc(C(C)(C)COC(=O)CCl)nn4)nn(C)c3c2)cn1 ; Cn1nc(-n2cc(CCOC(=O)CCl)nn2)c2ccc(-c3ccc(OC(F)(F)F)c(Cl)c3)cc21 ; COc1ccc(-c2ccc3c(-n4cc(C(C)(C)CNC(=O)CCl)nn4)nn(C)c3c2)cc1Cl] 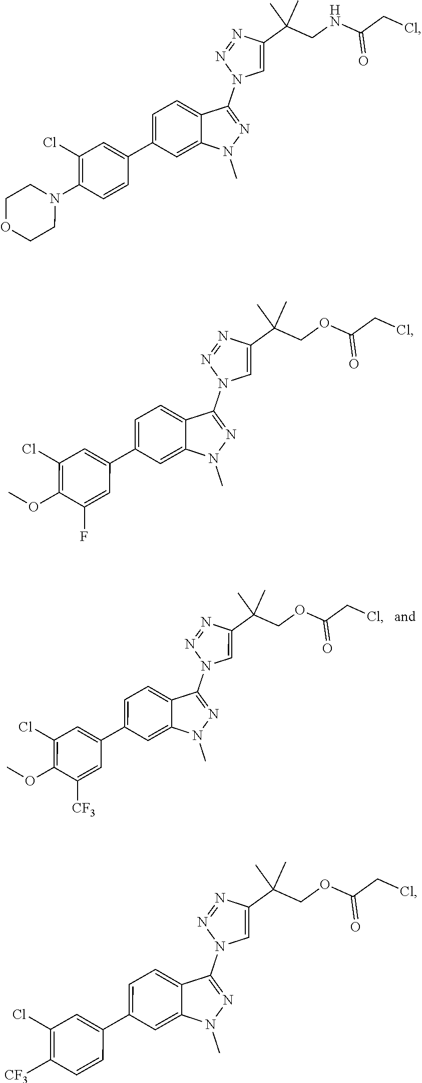 [SMILES: Cn1nc(-n2cc(C(C)(C)CNC(=O)CCl)nn2)c2ccc(-c3ccc(N4CCOCC4)c(Cl)c3)cc21 ; COc1c(F)cc(-c2ccc3c(-n4cc(C(C)(C)COC(=O)CCl)nn4)nn(C)c3c2)cc1Cl ; COc1c(Cl)cc(-c2ccc3c(-n4cc(C(C)(C)COC(=O)CCl)nn4)nn(C)c3c2)cc1C(F)(F)F ; "and" ; Cn1nc(-n2cc(C(C)(C)COC(=O)CCl)nn2)c2ccc(-c3ccc(C(F)(F)F)c(Cl)c3)cc21]
or an N-oxide, crystalline form, hydrate, or pharmaceutically acceptable salt thereof.

7. A compound according to formula (4): 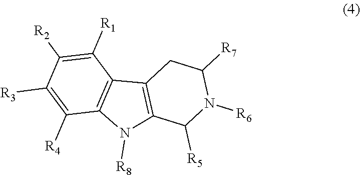
wherein:
R₁, R₂, R₃, R₄, R₅ and R₈ are independently selected from the group consisting of H, D, —OH, halo, ether, ester, furanyl, indole, indazole, pyrrole, pyrazole, pyridine, pyrimidine, naphthalene, indene, dibenzofuran, benzodioxole, amide, —(O)C(R), —C(O)OR, —NO₂, alkyl, aryl, alkyl-aryl, alkyl-heteroaryl, alkenyl, alkenyl-aryl, alkenyl-heteroaryl, peptide, and polypeptide, wherein the ether, ester, furanyl, indole, indazole, pyrrole, pyrazole, pyridine, pyrimidine, naphthalene, indene, dibenzofuran, benzodioxole, amide, alkyl, aryl, alkyl-aryl, alkyl-heteroaryl, alkenyl, alkenyl-aryl, alkenyl-heteroaryl, peptide, and polypeptide may be optionally substituted with an atom or a group selected from the group consisting of O, N, S, epoxy, —OH, halo, —CN, —NO₂, C₁₋₄alkyl, CF₃, —CF₃O, carbonyl, alkyl-aryl, thiol, methylmethanesulfonamide, methylthiol, dioxane, tetrahydropyzan, morpholinyl, piperazine, sulfonyl, oxazole, oxadiazole, dizaole, triazole, amide, ether, ester and combinations thereof; or R₁ and R₂ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₂ and R₃ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof; or R₃ and R₄ may together form a saturated or unsaturated C₃₋₁₂carbocycle that may be optionally substituted with an atom or a group selected from the group consisting of O, N, halo, C₁₋₄alkyl, CF₃, oxazole, oxadiazole, dizaole, triazole, amide, ether, ester and combinations thereof,
R₆ is selected from the group consisting of H, D, —OH, halo, C₁₋₄alkyl, CF₃, —(O)C(R), —C(O)OR, —NO₂, oxazole, oxadiazole, dizaole, triazole, amide, alcohol, ether, ester, and —C(O)C≡CSi(CH₃)₃,
R₇ is selected from the group consisting of H, D, —OH, halo, C₁₋₄alkyl, CF₃, —(O)C(R), —C(O)OR, —NO₂, oxazole, oxadiazole, dizaole, triazole, amide, alcohol, ether, and ester,
wherein R is selected from the group consisting of H, D, O, N, halo, oxazole, oxadiazole, dizaole, triazole, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of O, N, S, halo, C₁₋₄alkyl, CF₃, —CN and combinations thereof,
with the proviso that the compound is not 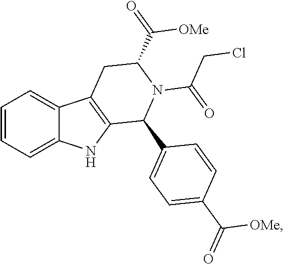 [SMILES: COC(=O)c1ccc([C@H]2c3[nH]c4ccccc4c3C[C@H](C(=O)OC)N2C(=O)CCl)cc1]
or an N-oxide, crystalline form, hydrate, or pharmaceutically acceptable salt thereof.

8. The compound according to claim 7 having a structure selected from the group consisting of: 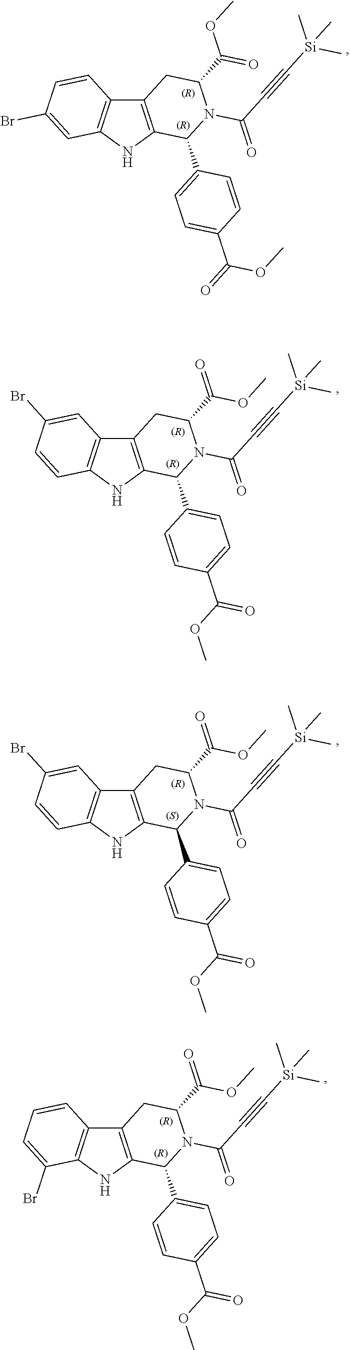 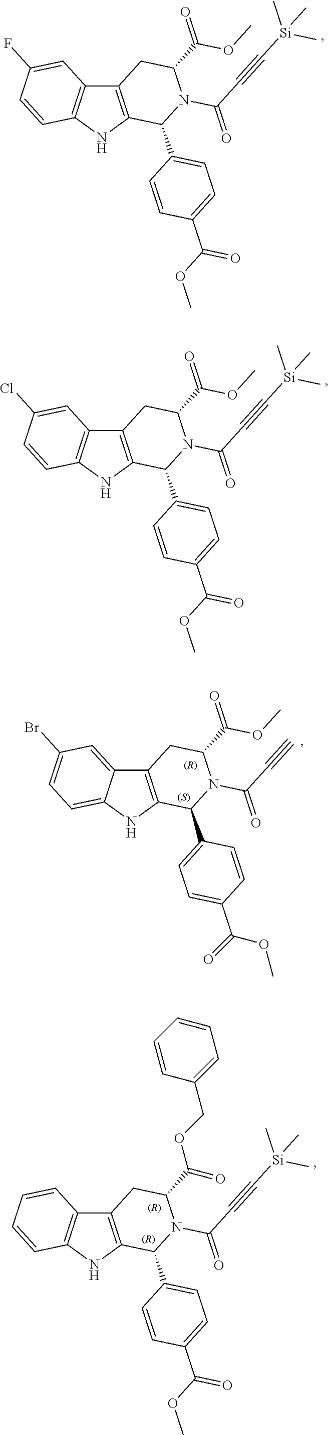 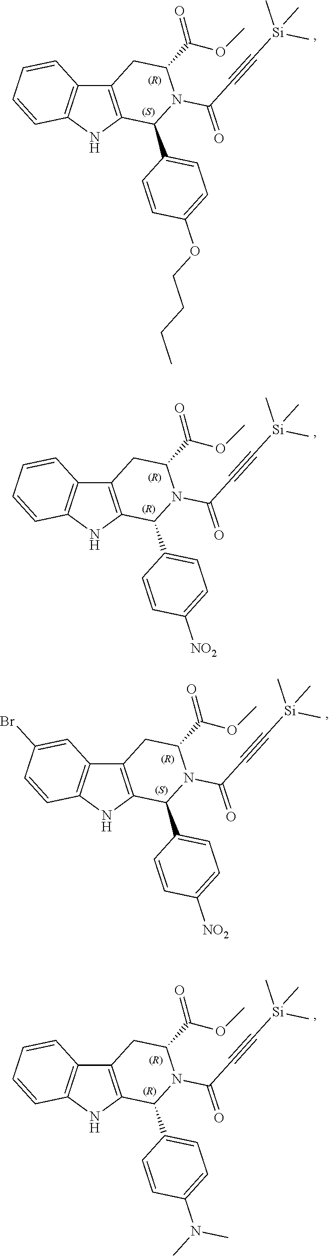 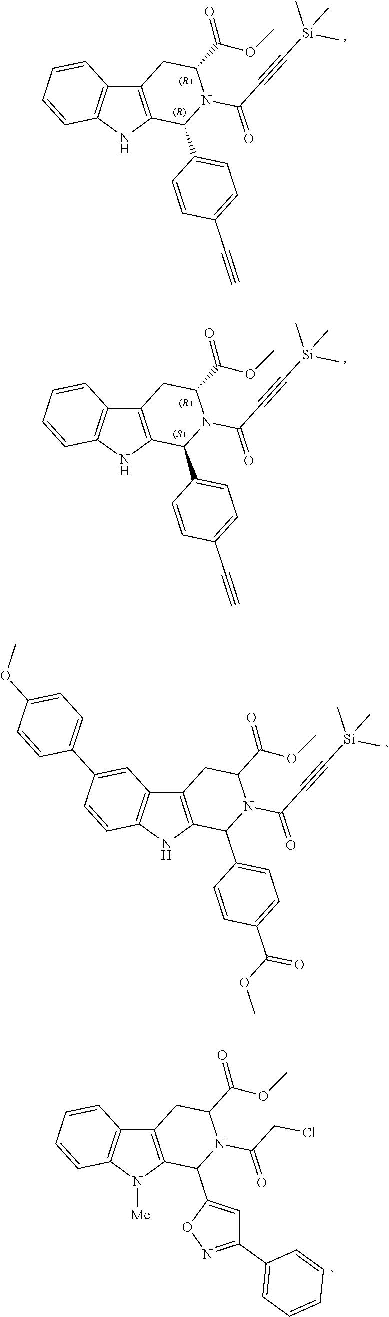 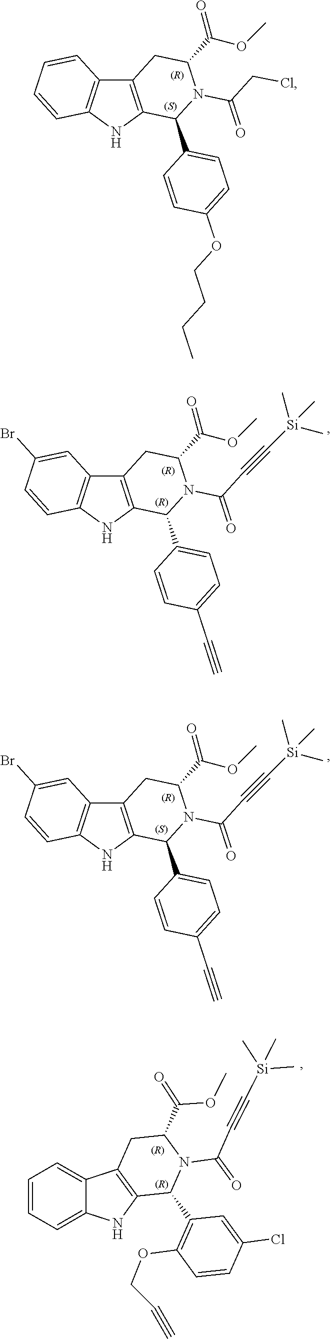 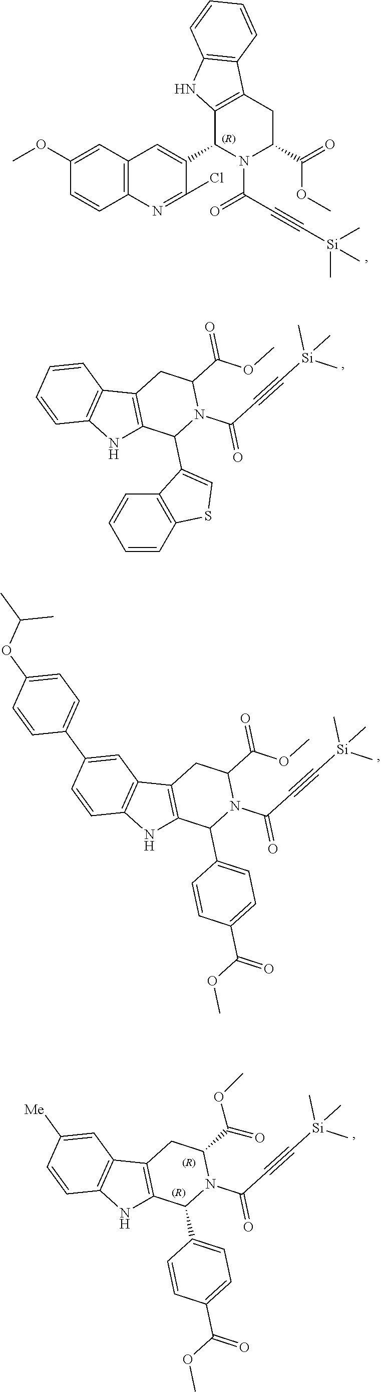 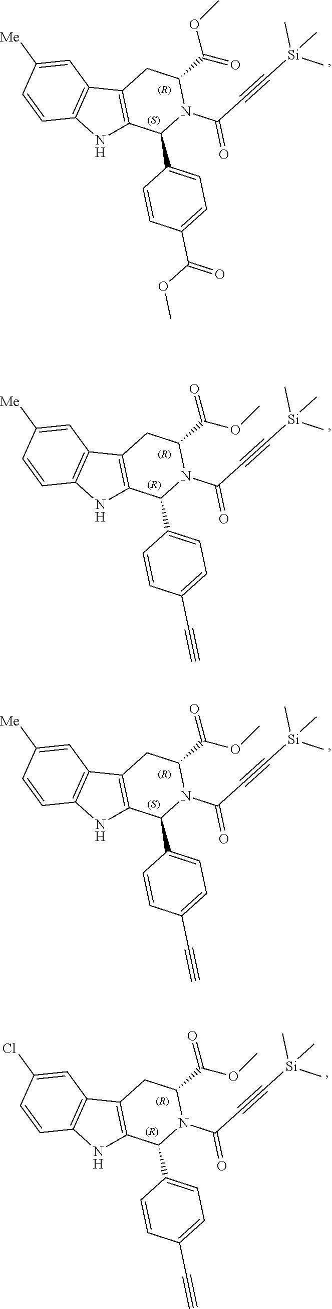 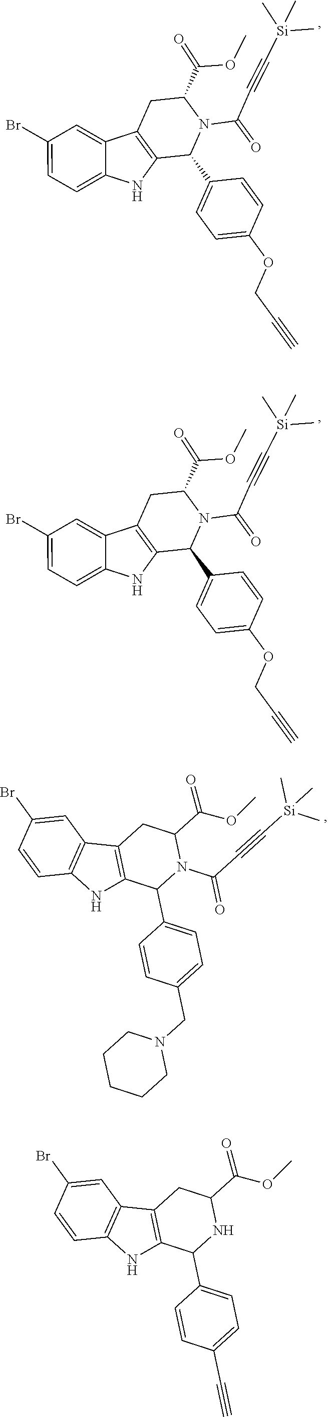 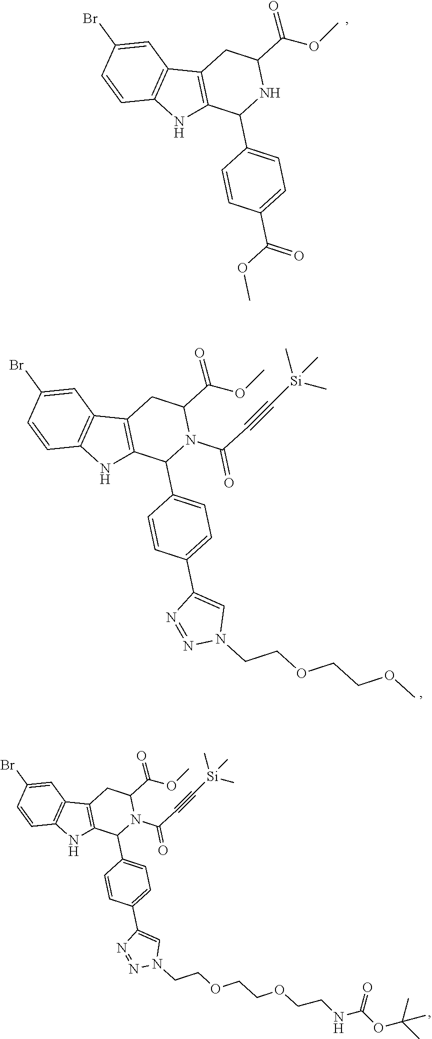 [SMILES: COC(=O)c1ccc(C2NC(C(=O)OC)Cc3c2[nH]c2ccc(Br)cc32)cc1 ; COCCOCCn1cc(-c2ccc(C3c4[nH]c5ccc(Br)cc5c4CC(C(=O)OC)N3C(=O)C#C[Si](C)(C)C)cc2)nn1 ; COC(=O)C1Cc2c([nH]c3ccc(Br)cc23)C(c2ccc(-c3cn(CCOCCOCCNC(=O)OC(C)(C)C)nn3)cc2)N1C(=O)C#C[Si](C)(C)C] 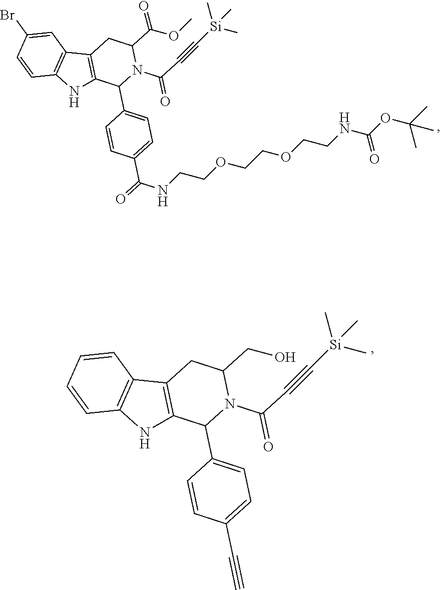 [SMILES: COC(=O)C1Cc2c([nH]c3ccc(Br)cc23)C(c2ccc(C(=O)NCCOCCOCCNC(=O)OC(C)(C)C)cc2)N1C(=O)C#C[Si](C)(C)C ; C#Cc1ccc(C2c3[nH]c4ccccc4c3CC(CO)N2C(=O)C#C[Si](C)(C)C)cc1] 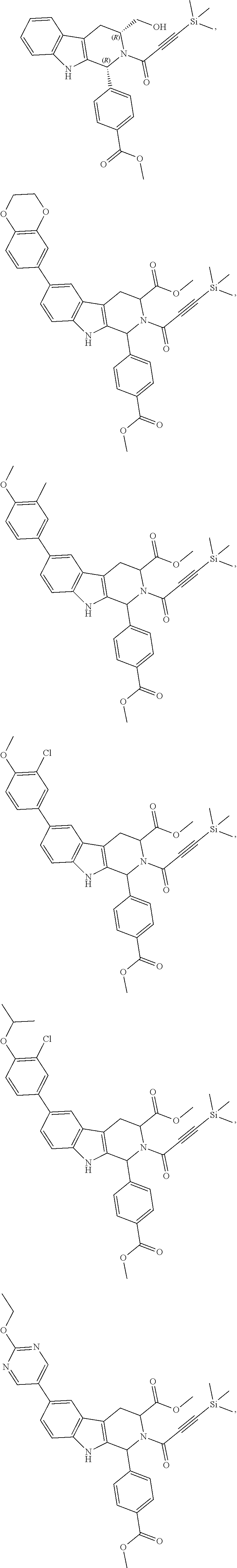 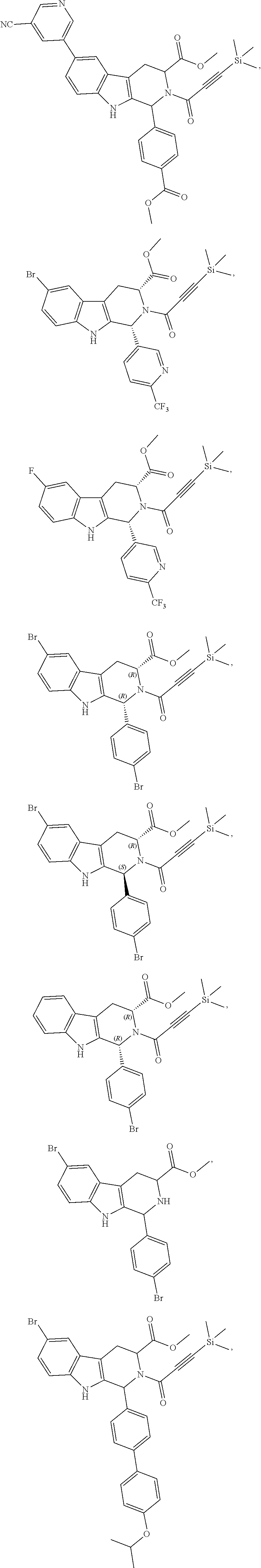 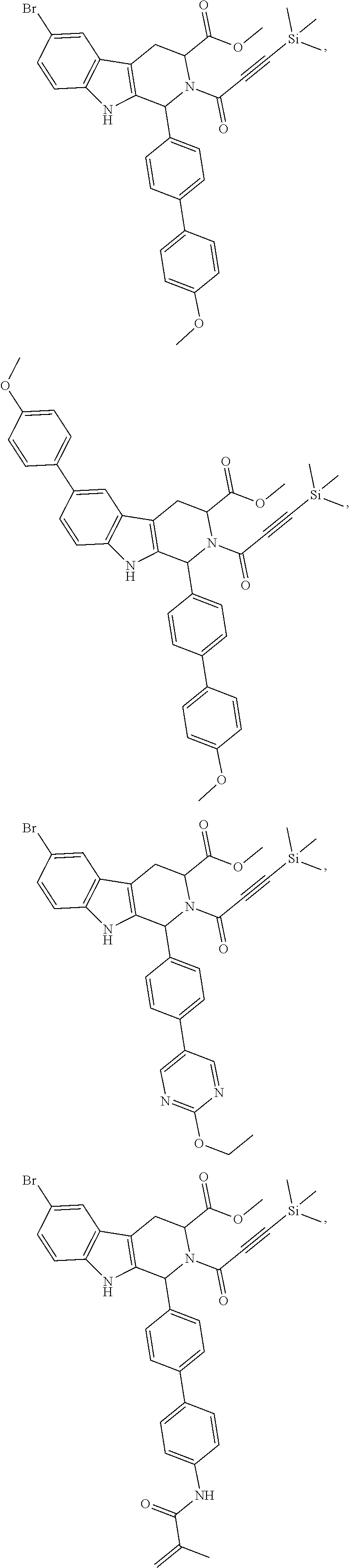 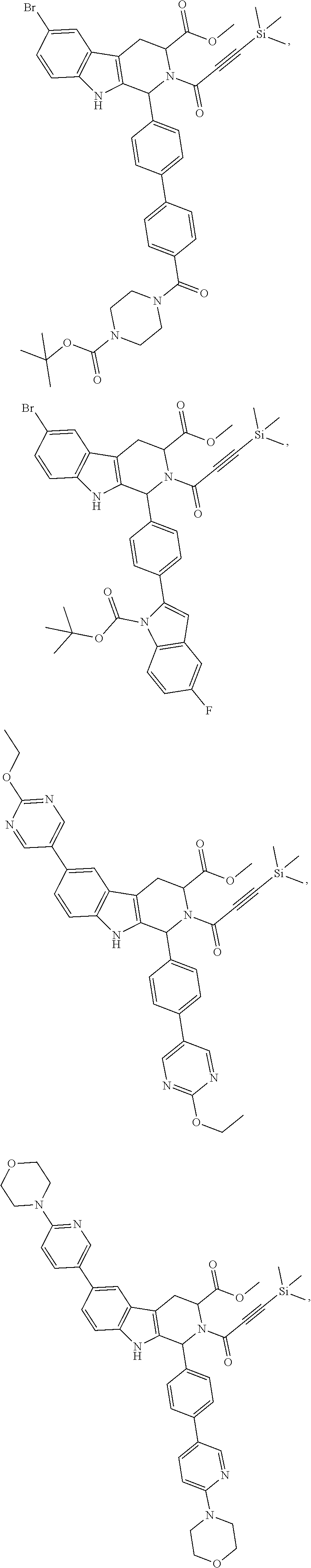 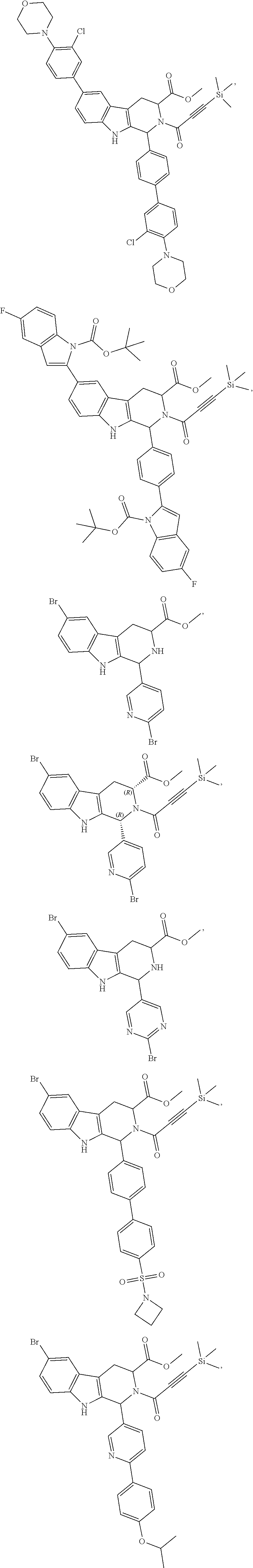 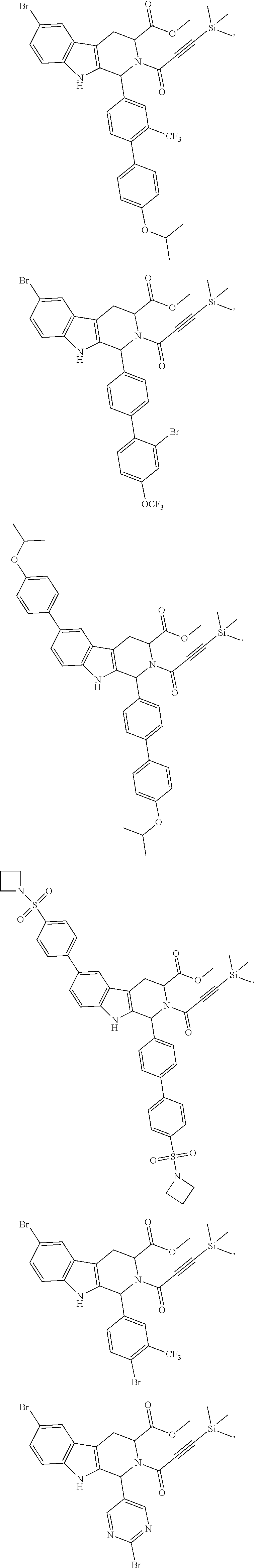 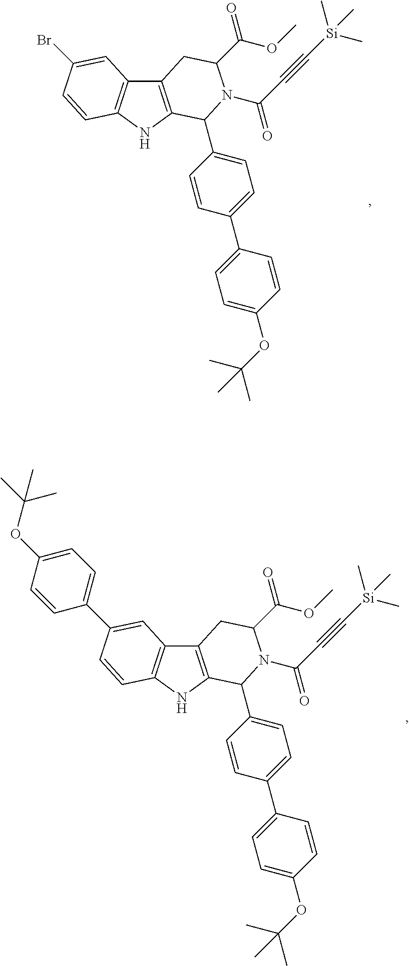 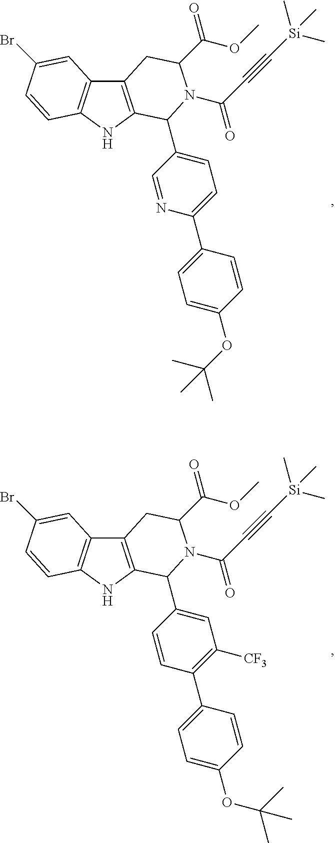 [SMILES: COC(=O)C1Cc2c([nH]c3ccc(Br)cc23)C(c2ccc(-c3ccc(OC(C)(C)C)cc3)nc2)N1C(=O)C#C[Si](C)(C)C ; COC(=O)C1Cc2c([nH]c3ccc(Br)cc23)C(c2ccc(-c3ccc(OC(C)(C)C)cc3)c(C(F)(F)F)c2)N1C(=O)C#C[Si](C)(C)C] 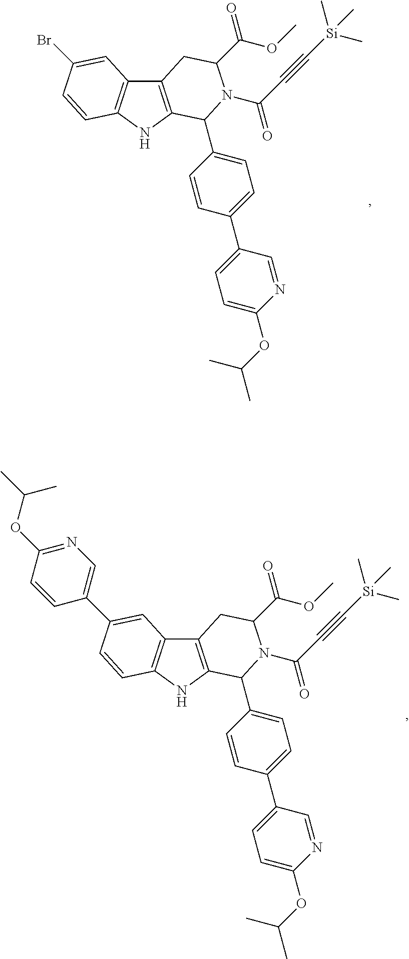 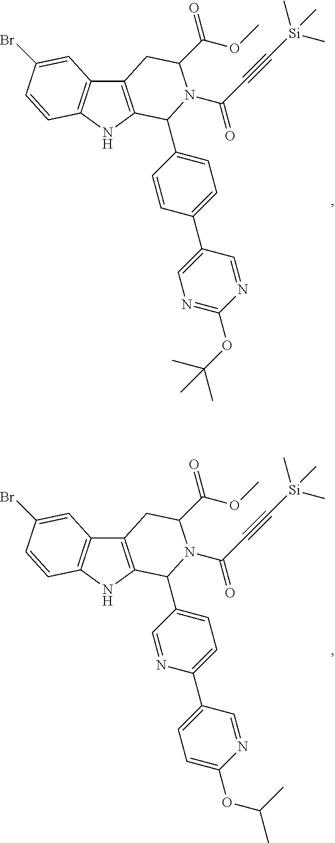 [SMILES: COC(=O)C1Cc2c([nH]c3ccc(Br)cc23)C(c2ccc(-c3cnc(OC(C)(C)C)nc3)cc2)N1C(=O)C#C[Si](C)(C)C ; COC(=O)C1Cc2c([nH]c3ccc(Br)cc23)C(c2ccc(-c3ccc(OC(C)C)nc3)nc2)N1C(=O)C#C[Si](C)(C)C] 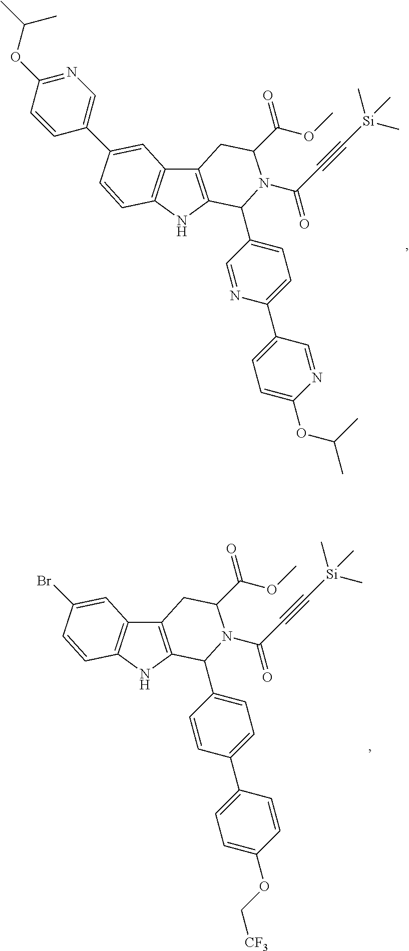 [SMILES: COC(=O)C1Cc2c([nH]c3ccc(-c4ccc(OC(C)C)nc4)cc23)C(c2ccc(-c3ccc(OC(C)C)nc3)nc2)N1C(=O)C#C[Si](C)(C)C ; COC(=O)C1Cc2c([nH]c3ccc(Br)cc23)C(c2ccc(-c3ccc(OCC(F)(F)F)cc3)cc2)N1C(=O)C#C[Si](C)(C)C] 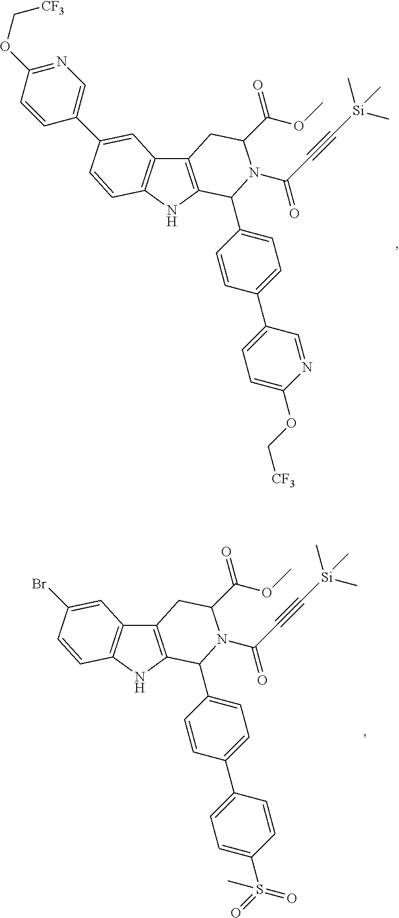 [SMILES: COC(=O)C1Cc2c([nH]c3ccc(-c4ccc(OCC(F)(F)F)nc4)cc23)C(c2ccc(-c3ccc(OCC(F)(F)F)nc3)cc2)N1C(=O)C#C[Si](C)(C)C ; COC(=O)C1Cc2c([nH]c3ccc(Br)cc23)C(c2ccc(-c3ccc(S(C)(=O)=O)cc3)cc2)N1C(=O)C#C[Si](C)(C)C] 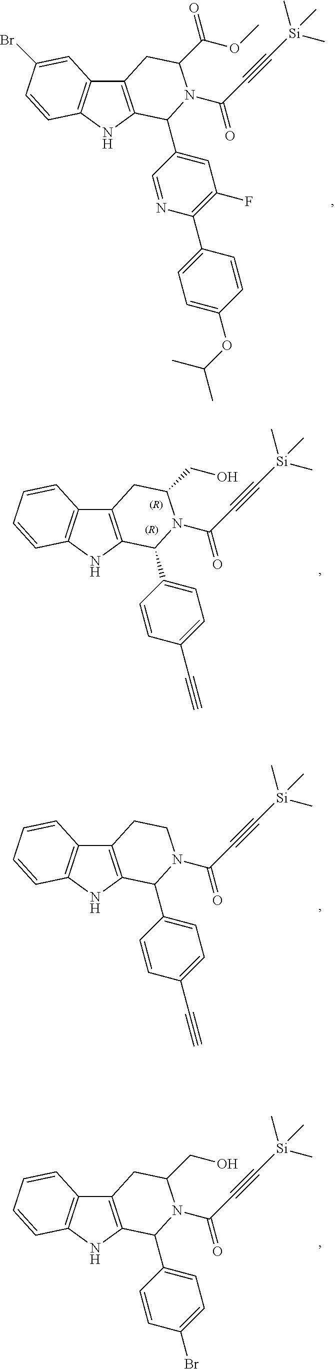 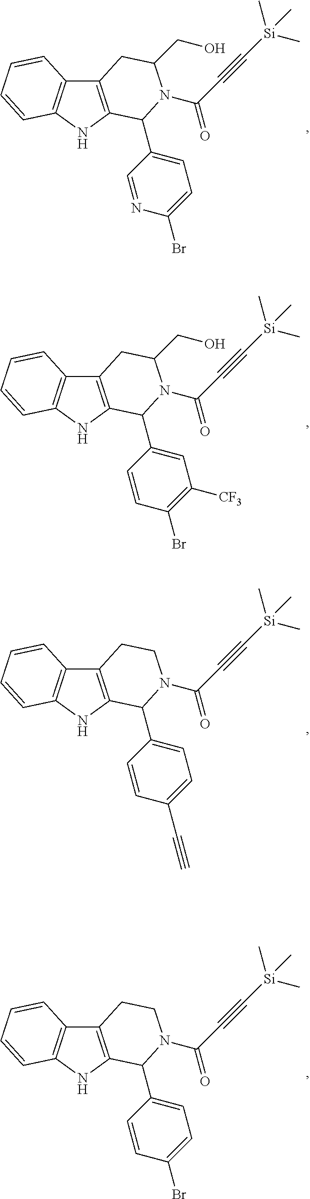 [SMILES: C[Si](C)(C)C#CC(=O)N1C(CO)Cc2c([nH]c3ccccc23)C1c1ccc(Br)nc1 ; C[Si](C)(C)C#CC(=O)N1C(CO)Cc2c([nH]c3ccccc23)C1c1ccc(Br)c(C(F)(F)F)c1 ; C#Cc1ccc(C2c3[nH]c4ccccc4c3CCN2C(=O)C#C[Si](C)(C)C)cc1 ; C[Si](C)(C)C#CC(=O)N1CCc2c([nH]c3ccccc23)C1c1ccc(Br)cc1] 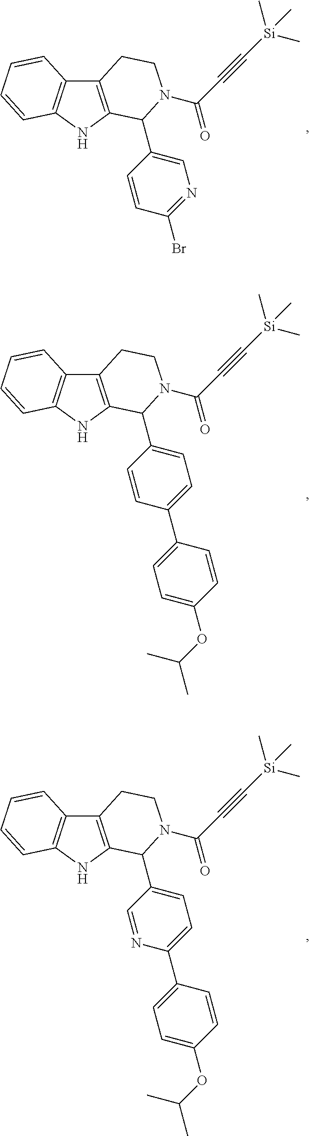 [SMILES: C[Si](C)(C)C#CC(=O)N1CCc2c([nH]c3ccccc23)C1c1ccc(Br)nc1 ; CC(C)Oc1ccc(-c2ccc(C3c4[nH]c5ccccc5c4CCN3C(=O)C#C[Si](C)(C)C)cc2)cc1 ; CC(C)Oc1ccc(-c2ccc(C3c4[nH]c5ccccc5c4CCN3C(=O)C#C[Si](C)(C)C)cn2)cc1] 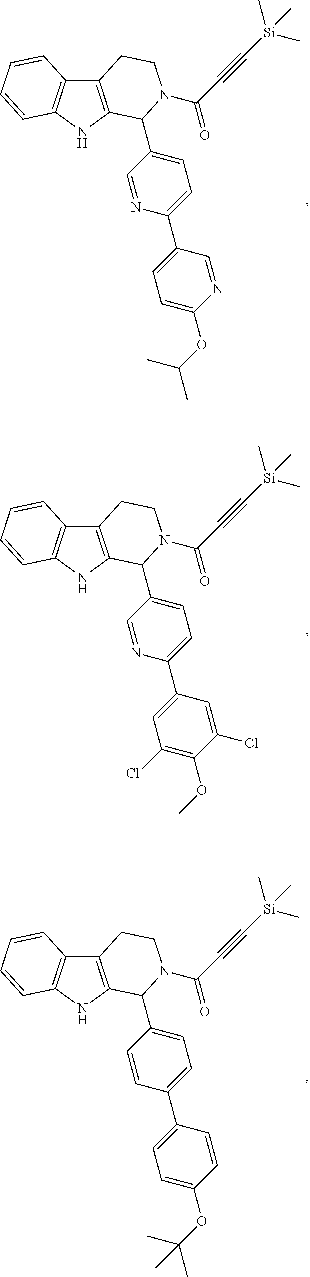 [SMILES: CC(C)Oc1ccc(-c2ccc(C3c4[nH]c5ccccc5c4CCN3C(=O)C#C[Si](C)(C)C)cn2)cn1 ; COc1c(Cl)cc(-c2ccc(C3c4[nH]c5ccccc5c4CCN3C(=O)C#C[Si](C)(C)C)cn2)cc1Cl ; CC(C)(C)Oc1ccc(-c2ccc(C3c4[nH]c5ccccc5c4CCN3C(=O)C#C[Si](C)(C)C)cc2)cc1] 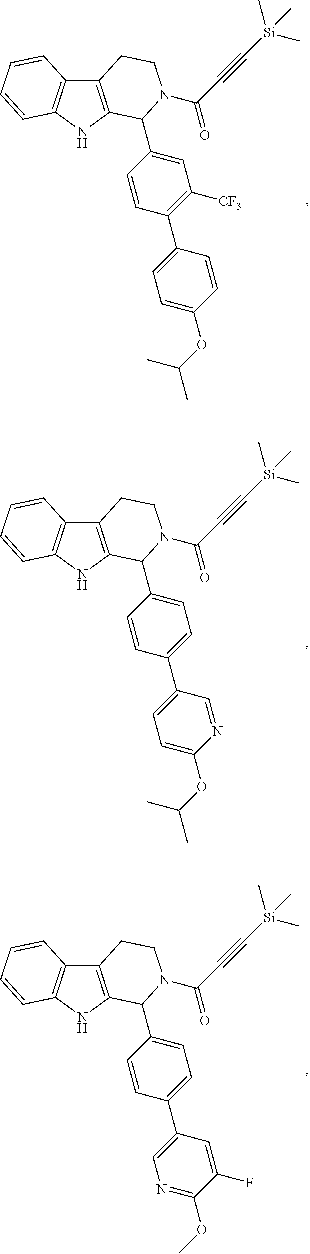 [SMILES: CC(C)Oc1ccc(-c2ccc(C3c4[nH]c5ccccc5c4CCN3C(=O)C#C[Si](C)(C)C)cc2C(F)(F)F)cc1 ; CC(C)Oc1ccc(-c2ccc(C3c4[nH]c5ccccc5c4CCN3C(=O)C#C[Si](C)(C)C)cc2)cn1 ; COc1ncc(-c2ccc(C3c4[nH]c5ccccc5c4CCN3C(=O)C#C[Si](C)(C)C)cc2)cc1F] 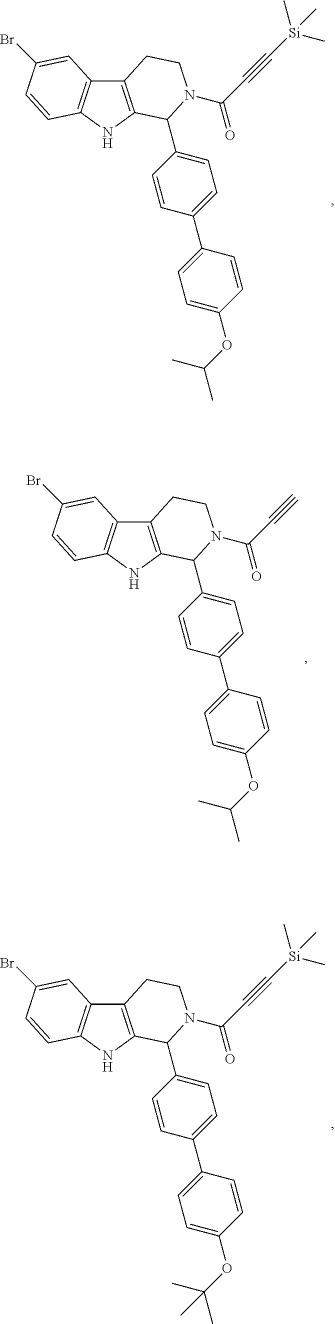 [SMILES: CC(C)Oc1ccc(-c2ccc(C3c4[nH]c5ccc(Br)cc5c4CCN3C(=O)C#C[Si](C)(C)C)cc2)cc1 ; C#CC(=O)N1CCc2c([nH]c3ccc(Br)cc23)C1c1ccc(-c2ccc(OC(C)C)cc2)cc1 ; CC(C)(C)Oc1ccc(-c2ccc(C3c4[nH]c5ccc(Br)cc5c4CCN3C(=O)C#C[Si](C)(C)C)cc2)cc1] 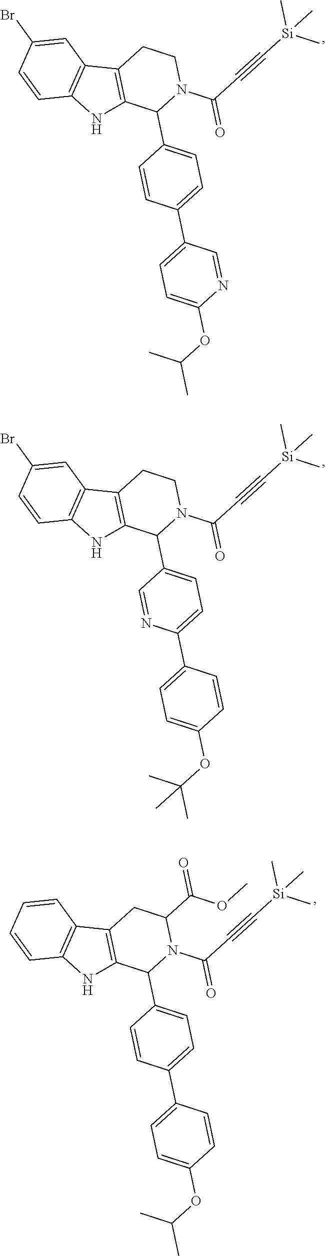 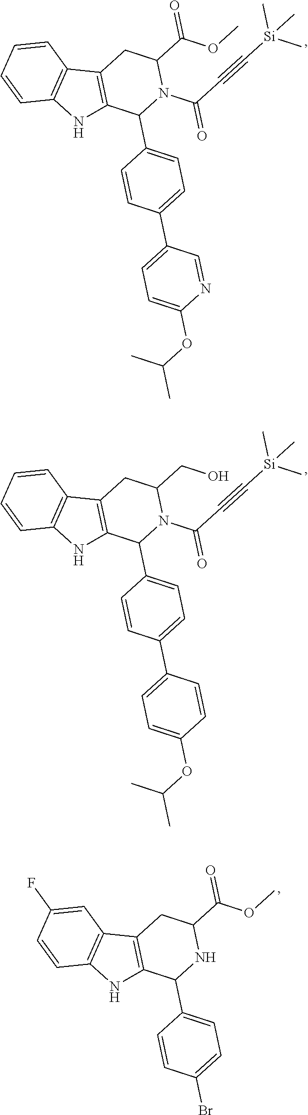 [SMILES: COC(=O)C1Cc2c([nH]c3ccccc23)C(c2ccc(-c3ccc(OC(C)C)nc3)cc2)N1C(=O)C#C[Si](C)(C)C ; CC(C)Oc1ccc(-c2ccc(C3c4[nH]c5ccccc5c4CC(CO)N3C(=O)C#C[Si](C)(C)C)cc2)cc1 ; COC(=O)C1Cc2c([nH]c3ccc(F)cc23)C(c2ccc(Br)cc2)N1] 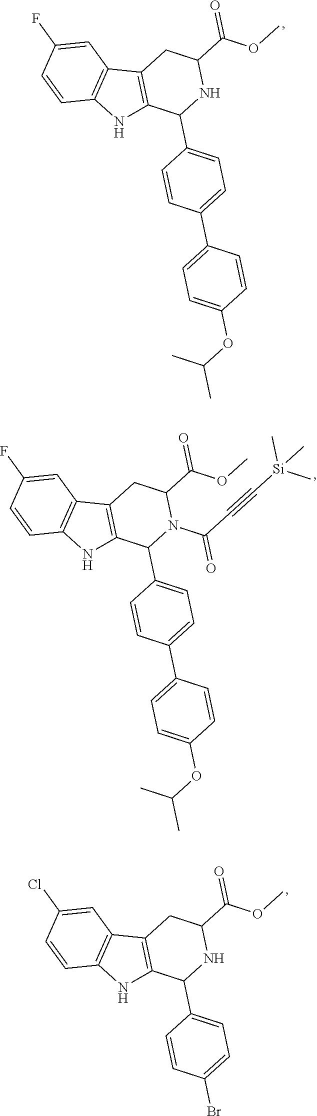 [SMILES: COC(=O)C1Cc2c([nH]c3ccc(F)cc23)C(c2ccc(-c3ccc(OC(C)C)cc3)cc2)N1 ; COC(=O)C1Cc2c([nH]c3ccc(F)cc23)C(c2ccc(-c3ccc(OC(C)C)cc3)cc2)N1C(=O)C#C[Si](C)(C)C ; COC(=O)C1Cc2c([nH]c3ccc(Cl)cc23)C(c2ccc(Br)cc2)N1] 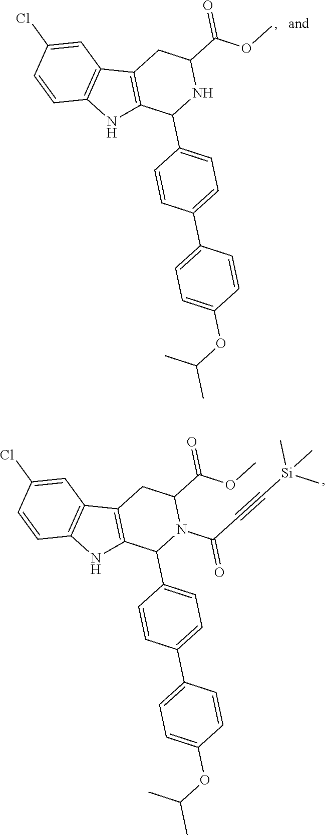
or an N-oxide, crystalline form, hydrate, or pharmaceutically acceptable salt thereof.

9. The compound according to claim 7 having the structure of: 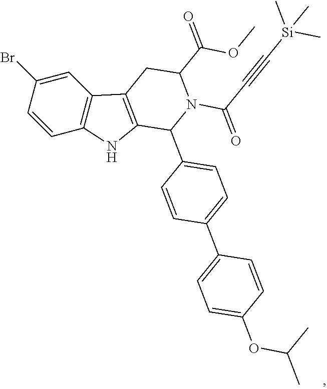 [SMILES: COC(=O)C1Cc2c([nH]c3ccc(Br)cc23)C(c2ccc(-c3ccc(OC(C)C)cc3)cc2)N1C(=O)C#C[Si](C)(C)C]
or an N-oxide, crystalline form, hydrate, or pharmaceutically acceptable salt thereof.

10. A compound according to formula (5): 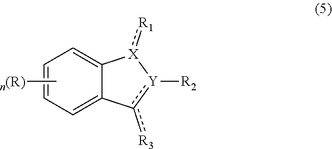
wherein:
a dashed line indicates the presence of an optional double bond;
n=2, 3, or 4;
X and Y are independently selected from the group consisting of C, N, S and O;
R₁, R₂, and R₅ are independently selected from the group consisting of no atom, H, D, O, N, halo, ether, ester, amide, C(O), (O)C(R), C(O) O, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl, wherein the ether, ester, C₁₋₆alkyl, C₁₋₆alkyl-aryl, C₁₋₆alkyl-heteroaryl, C₁₋₆alkenyl, C₁₋₆alkenyl-aryl, and C₁₋₆alkenyl-heteroaryl may be optionally substituted with an atom or a group selected from the group consisting of N, O, Sn, halo, C₁₋₄alkyl, CF₃, and combinations thereof,
wherein R is selected from the group consisting of H, D, O, N, halo, C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle, wherein the C₁₋₆alkyl, C₁₋₆alkyl-aryl, C₁₋₆alkyl-heteroaryl, C₁₋₆alkenyl, C₁₋₆alkenyl-aryl, C₁₋₆alkenyl-heteroaryl and C₃₋₁₂carbocycle may be optionally substituted with an atom or a group selected from the group consisting of halo, C₁₋₄alkyl, CF₃, and combinations thereof,
with the proviso that the compound is not 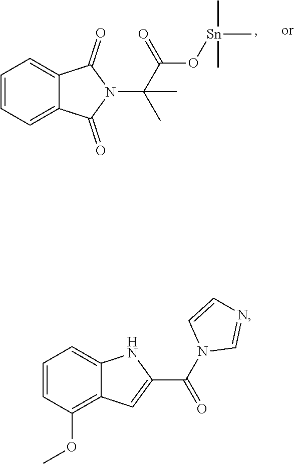 [SMILES: CC(C)(C(=O)[O][Sn]([CH3])([CH3])[CH3])N1C(=O)c2ccccc2C1=O ; COc1cccc2[nH]c(C(=O)n3ccnc3)cc12]
or an N-oxide, crystalline form, hydrate, or pharmaceutically acceptable salt thereof.

11. A composition comprising one or more compounds of claim 1 and a pharmaceutically acceptable carrier, adjuvant or vehicle.

12. A method for treating or ameliorating the effects of a glutathione peroxidase 4 (GPX4)-associated disease in a subject in need thereof, comprising administering to the subject an effective amount of one or more compounds of claim 1.

13. The method of claim 12, wherein the GPX4-associated disease is selected from the group consisting of a cancer, a neurotic disorder, a neurodegenerative disorder, spondylometaphyseal dysplasia, mixed cerebral palsy, pontocerebellar hypoplasia, and male infertility.

14. The method of claim 13, wherein the GPX4-associated disease is a cancer.

15. The method of claim 14, wherein the cancer is selected from the group consisting of hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma, and non-small cell lung carcinoma.

16. The method of claim 14, wherein the cancer is hepatocellular carcinoma.

17. The method of claim 12, wherein the subject is a mammal.

18. The method of claim 17, wherein the mammal is selected from the group consisting of humans, veterinary animals, and agricultural animals.

19. The method of claim 12, wherein the subject is a human.

20. The method of claim 14, wherein the cancer is metastatic.

21. The method of claim 14, wherein the cancer is under epithelial-to-mesenchymal (EMT) transition.

22. The method of claim 14, wherein the cancer is hypersensitive to ferroptosis.

23. The method of claim 14, wherein the cancer is refractory to standard cancer treatment.

24. The method of claim 23, wherein the standard cancer treatment comprises chemotherapy, radiation therapy, targeted therapy, immunotherapy, and combinations thereof.

25. A method for modulating the activity of glutathione peroxidase 4 (GPX4) in a subject in need thereof, comprising administering to the subject an effective amount of one or more compounds of claim 1.

26. The method of claim 25, wherein the modulation comprises inhibiting GPX4 activity.

27. A method for increasing the level of peroxide in a subject in need thereof, comprising administering to the subject an effective amount of one or more compounds of claim 1.

28. The method of claim 27, wherein the peroxide is selected from the group consisting of hydrogen peroxide, organic hydroperoxide, lipid peroxide, and combinations thereof.

29. A method for inducing ferroptosis in a cell, comprising contacting the cell with an effective amount of one or more compounds of claim 1.

30. The method of claim 29, wherein the cell has aberrant lipid accumulation.

31. The method of claim 29, wherein the cell is a cancer cell.

32. The method of claim 31, wherein the cancer is selected from the group consisting of hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma, and non-small cell lung carcinoma.

33. The method of claim 31, wherein the cancer is hepatocellular carcinoma.

34. The method of claim 29, wherein the cell is a human cell.

35. The method of claim 32, wherein the cancer cell is metastatic.

36. The method of claim 32, wherein the cancer cell is under epithelial-to-mesenchymal (EMT) transition.

37. The method of claim 32, wherein the cancer cell is hypersensitive to ferroptosis.

38. The method of claim 32, wherein the cancer cell is refractory to standard cancer treatment.

39. The method of claim 38, wherein the standard cancer treatment comprises chemotherapy, radiation therapy, targeted therapy, immunotherapy, and combinations thereof.

40. A method for treating or ameliorating the effects of a cancer in a subject in need thereof, comprising administering to the subject i) an effective amount of a first anti-cancer agent, which is one or more compounds of claim 1, and ii) an effective amount of a second anti-cancer agent.

41. The method of claim 40, wherein the second anti-cancer agent is selected from the group consisting of chemotherapy, radiation therapy, targeted therapy, immunotherapy, and combinations thereof.

42. The method of claim 40, wherein the second anti-cancer agent is an immunotherapy.

43. The method of claim 42, wherein the immunotherapy is selected from ipilimumab, pembrolizumab, nivolumab, atezolizumab, avelumab, durvalumab, cemiplimab, ofatumumab, blinatumomab, daratumumab, elotuzumab, obinutuzumab, talimogene laherparepvec, necitumumab, lenalidomide, dinutuximab, and combinations thereof.

44. The method of claim 40, wherein the subject is a human.

45. The method of claim 40, wherein the cancer is metastatic.

46. The method of claim 40, wherein the cancer is under epithelial-to-mesenchymal (EMT) transition.

47. The method of claim 40, wherein the cancer is hypersensitive to ferroptosis.

48. The method of claim 47, wherein the hypersensitivity to ferroptosis is identified by NADPH abundance, GCH1 expression, NF2-YAP activity, EMT signature, and GPX4 expression.

49. The method of claim 40, wherein the cancer is refractory to standard cancer treatment.

50. The method of claim 40, wherein the cancer is selected from the group consisting of hepatocellular carcinoma, sarcoma, glioma, renal cell carcinoma, ovarian cancer, prostate cancer, breast cancer, pancreatic cancer, melanoma, colon cancer, diffuse large B cell lymphoma, leukemia, lung cancer, clear-cell carcinoma, and non-small cell lung carcinoma.

51. The method of claim 40, wherein the cancer is hepatocellular carcinoma.

52. The method of claim 40, wherein the first anti-cancer agent is administered to the subject before, concurrently with, or after the second anti-cancer agent.

53. A kit for treating or ameliorating the effects of a glutathione peroxidase 4 (GPX4)-associated disease in a subject in need thereof, comprising an effective amount of one or more compounds of claim 1, packaged with its instructions for use.
